SEQUENCE LISTING (4 sequences)

SEQ ID NO: 1 - protein, 216 aa, Synthetic construct
  REGION 1..216: note = His10-hK-Ras with mutations G12C-C118S-D126E-T127S-K128R (mutations according to numbering in P01116-2) before cleavage by TEV protease
MGHHHHHHHHHHSSGHIEGRHMLETSLYKKAGSDYDIPTTENLYFQGMTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKSDLPSRTVESRQAQDLARSYGIPFIETSAKTRQGVDDAFYTLVREIRKHKEK

SEQ ID NO: 2 - protein, 170 aa, Synthetic construct
  REGION 1..170: note = His10-hK-Ras with mutations G12C-C118S-D126E-T127S-K128R after cleavage by TEV protease
GMTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKSDLPSRTVESRQAQDLARSYGIPFIETSAKTRQGVDDAFYTLVREIRKHKEK

SEQ ID NO: 3 - protein, 533 aa, Synthetic construct
  REGION 1..533: note = hSOS1 with N-terminal His tag (His10-hSOS1) before cleavage by TEV protease
MGHHHHHHHHHHSSGHIEGRHMLETSLYKKAGSDYDIPTTENLYFQGEEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT

SEQ ID NO: 4 - protein, 487 aa, Synthetic construct
  REGION 1..487: note = hSOS1 after cleavage by TEV protease
GEEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT

(19) United States
(12) Patent Application Publication
(10) Pub. No.: US 2024/0083857 A1
(43) Pub. Date: Mar. 14, 2024

(54) 2-Methyl-Quinazolines

(71) Applicant: Bayer Pharma Aktiengesellschaft, Berlin (DE)

(72) Inventors: Lars WORTMANN, Berlin (DE); Brice SAUTIER, Roques (FR); Knut EIS, Berlin (DE); Hans BRIEM, Berlin (DE); Niels BOHNKE, Berlin (DE); Franz VON NUSSBAUM, Berlin (DE); Roman HILLIG, Berlin (DE); Benjamin BADER, Berlin (DE); Jens SCHRODER, Berlin (DE); Kirstin PETERSEN, Berlin (DE); Philip LIENAU, Berlin (DE); Antje Margret WENGNER, Berlin (DE); Dieter MOOSMAYER, Berlin (DE); Qiuwen WANG, Beijing (CN); Hans SCHICK, Berlin (DE)

(21) Appl. No.: 18/350,246

(22) Filed: Jul. 11, 2023

Related U.S. Application Data
(63) Continuation of application No. 16/496,825, filed on Sep. 23, 2019, now abandoned.

(30) Foreign Application Priority Data
Mar. 21, 2017 (WO) PCT/CN2017/077501

(51) Int. Cl. C07D 239/94; C07D 401/12; C07D 403/12; C07D 405/12; C07D 409/12; C07D 409/14; C07D 413/12; C07D 413/14; C07D 417/12; C07D 471/04; C07D 405/14; C07D 405/10; C07D 495/04; C07D 403/04; C07D 487/04; A61P 35/00

(52) U.S. Cl. CPC C07D 239/94; C07D 401/12; C07D 403/12; C07D 405/12; C07D 409/12; C07D 409/14; C07D 413/12; C07D 413/14; C07D 417/12; C07D 471/04; C07D 405/14; C07D 405/10; C07D 495/04; C07D 403/04; C07D 487/04; A61P 35/00

(57) ABSTRACT

The present invention describes 2-methyl-quinazoline compounds of general formula (I), methods of preparing said compounds, intermediate compounds useful for preparing said compounds, pharmaceutical compositions and combinations comprising said compounds, and the use of said compounds for manufacturing pharmaceutical compositions. The 2-methyl substituted quinazoline compounds of general formula (I) effectively and selectively inhibit the Ras-Sos interaction without significantly targeting the EGFR receptor. They are therefore useful for the treatment or prophylaxis of diseases, in particular of hyperproliferative disorders, such as cancer as a sole agent or in combination with other active ingredients.

### REFERENCE TO RELATED APPLICATIONS

The present application is a Continuation application of U.S. Application No. 16,496,825, filed Sep. 23, 2019, which is the 371 U.S. National Stage Application of International Application No. PCT/EP2018/056824, filed Mar. 19, 2018, which claims the benefit of and priority to PCT/CN2017/077501, filed Mar. 21, 2017. The entire contents of each of these applications are hereby incorporated by reference for all relevant purposes.

### SEQUENCE LISTING

This application contains references to amino acid sequences and/or nucleic acid sequences which have been submitted concurrently herewith as the sequence listing .xml file entitled “000020uscob_SequenceListing.xml”, file size 7.56 KiloBytes (KB), created on Jul. 10, 2023. The aforementioned sequence listing is hereby incorporated by reference in its entirety pursuant to 37 C.F.R. § 1.52(e)(5).

The present invention covers 2-methyl-quinazoline compounds of general formula (I) as described and defined herein, methods of preparing said compounds, intermediate compounds useful for preparing said compounds, pharmaceutical compositions and combinations comprising said compounds, and the use of said compounds for manufacturing pharmaceutical compositions for the treatment or prophylaxis of diseases, in particular of hyperproliferative disorders, as a sole agent or in combination with other active ingredients.

### BACKGROUND

The present invention covers 2-methyl-quinazoline compounds of general formula (I) which inhibit the Ras-Sos interaction.

In the 2-position substituted quinazoline compounds are described e.g. in EP 0326328, EP 0326329, WO93/007124, WO2003/087098 and U.S. Pat. No. 5,236,925. These compounds are either not described as pharmaceutically active compounds or, if they are described as pharmacologically active compounds, they are described as compounds having affinity to the Epidermal Growth Factor Receptor (EGFR).

In the majority (45-100%) of patients receiving EGFR inhibitors skin toxicity is a class-specific side effect that is typically manifested as a papulopustular rash. The skin toxicity is related to the inhibition of EGFR in the skin, which is crucial for the normal development and physiology of the epidermis.

However, the state of the art does not describe:
the 2-methyl substituted quinazoline compounds of general formula (I) of the present invention as described and defined herein, i.e. compounds having a quinazoline core bearing a methyl group on the carbon atom 2 which effectively and selectively inhibit the Ras-Sos interaction without significantly targeting the EGFR receptor.

Ras proteins play an important role in human cancer. Mutations in Ras proteins can be found in 20-30% of all human tumors and are recognized as tumorigenic drivers especially in lung, colorectal and pancreatic cancers (Malumbres & Barbacid 2002 Nature Reviews Cancer, Pylayeva-Gupta et al. 2011 Nature Reviews Cancer). Three human Ras genes are known that encode four different Ras proteins of 21 kDa size: H-Ras, N-Ras, and two splice variants of K-Ras, namely K-Ras 4A and K-Ras-4B. All Ras isoforms are highly conserved within the GTP-binding domain and differ mainly in the hypervariable C-terminal region. The C-termini of the different Ras-isoforms are posttranslationally modified by lipidation (farnesylation, palmitoylation) to facilitate membrane anchorage. The localization of Ras-proteins at the cytoplasmic membrane provides vicinity to transmembrane growth receptors and has been shown to be essential for transmitting growth signals from extracellular growth factor binding to intracellular downstream pathways. A variety of upstream signals may activate Ras proteins depending on the cellular context, such as epidermal growth factor receptor (EGFR), platelet-derived growth factor receptor (PDGFR), nerve growth factor receptor (NGFR) and others. Activated Ras can signal through various downstream pathways, e.g. the Raf-MEK-ERK or the PI3K-PDK1-Akt pathways.

On the molecular level, Ras proteins function as molecular switches. By binding GTP and GDP they exist in an active (GTP-bound) and inactive (GDP-bound) state in the cell. Active GTP-loaded Ras recruits other proteins by binding of their cognate Ras-binding domains (RBDs) resulting in activation of the effector protein followed by downstream signalling events of diverse functions, e.g. cytoskeletal rearrangements or transcriptional activation. The activity status of Ras is tightly regulated by guanine nucleotide exchange factors (GEFs) and GTPase activating proteins (GAPs). GEFs function as activators of Ras by promoting the nucleotide exchange from GDP to GTP. GAPs deactivate Ras-GTP by catalyzing the hydrolysis of the bound GTP to GDP. In a cancer cell, point mutations, typically within the GTP-binding region at codon 12, eliminate the ability of RAS to efficiently hydrolyse bound GTP, even in the presence of a GAP. Therefore, cancer cells comprise increased levels of active mutated Ras-GTP, which is thought to be a key factor for driving cancer cell proliferation.

Three main families of RAS-specific GEFs have been identified so far (reviewed in Vigil 2010 Nature Reviews Cancer; Rojas et al 2011, Genes & Cancer 2(3) 298-305). There are two son of sevenless proteins (SOS1 and SOS2), 4 different isoforms of Ras guanine nucleotide releasing proteins (Ras-GRP1-4) and two Ras guanine nucleotide releasing factors (Ras-GRF1 and 2). The SOS proteins are ubiquitously expressed and are recruited to sites of activated growth factors. Ras-GRFs are expressed mainly in the nervous system, where they are involved in Calcium-dependent activation of Ras. In contrast, Ras GRP proteins are expressed in hematopoietic cells and act in concert with non-receptor tyrosine kinases. In the context of cancer, mainly SOS proteins have been found to be involved.

Targeting Ras for cancer therapy has been a dream since the 1990s (Downward 2002 Nature Reviews Cancer, Krens et al. 2010 Drug Discovery Today). Due to the compact nature, the high affinity towards GDP and GTP in combination with high intracellular GTP concentrations, the Ras protein itself has always been considered to be undruggable, i.e. the chance to identify small chemical molecules that would bind to and inhibit active Ras was rated extremely low. Alternative approaches have been undertaken to reduce Ras signaling, e.g. by addressing more promising drug targets such as enzymes involved in the posttranslational modification of Ras proteins, especially farnesyltransferase and geranylgeranyltransferase (Berndt 2011 Nature Reviews Cancer). Inhibitors of farnesyltransferase (FTIs) were identified and developed with promising antitumor effects in preclinical models. Unexpectedly, in clinical trials these inhibitors have been of limited efficacy. Targeting upstream and downstream kinases involved in Ras signaling pathways has been more successful. Several drugs are and have been in clinical trials that inhibit different kinases, e.g. EGFR, Raf, MEK, Akt, PI3K (Takashima & Faller 2013 Expert Opin. Ther. Targets). Marketed cancer drugs are available that inhibit Raf, EGFR or MEK.

Nevertheless, there is still a large unmet need for the treatment of Ras-dependent tumors that are resistant against current therapies. Many research groups have been active to identify small molecules that target Ras directly (Ras small molecules have been reviewed in: Cox et al. 2014 Nature Reviews Drug Discovery; Stephen et al. 2014 Cancer Cell; Hattum & Waldmann 2014 Chemistry & Biology, Spiegel et al. 2014 Nature Chemical Biology). One group of inhibitors comprises small molecules that inhibit the interaction of Ras with its effectors Raf or PI3K. Another group of compounds acts as covalent inhibitors of a specific cysteine mutant form of K-Ras (glycine to cysteine point mutation G12C). The specific targeting of the Ras-G12C mutant might have the benefit of reduced side effects, as the wildtype Ras proteins should not be affected. Furthermore, several reports show small molecules and peptides that interrupt the GEF assisted activation of Ras. There seem to be several different binding sites possible that result in this mode of action. Inhibitors may bind to Ras or to the GEF in an allosteric or orthosteric fashion. All these approaches of direct Ras-targeting are in preclinical research stage and the affinity of published small molecule inhibitors is still in the micromolar range. Stabilized peptides have been shown to be active in the nanomolar range. (Leshchiner et al. 2015 PNAS). Their usefulness as drugs in a clinical setting has to be awaited.

The Epidermal Growth Factor Receptor (EGFR) is a tyrosine kinase (TK) receptor that is activated upon binding to the Epidermal Growth Factor and other growth factor ligands, triggering several downstream pathways, including RAS/MAPK, PI3K/Akt and STAT that regulate different cellular processes, including DNA synthesis and proliferation (Russo A, Oncotarget. 4254, 2015). The family of HER (ErbB) receptor tyrosine kinases consists of four members, ie, epidermal growth factor receptors [EGFR (HER1 or ErbB1), HER2 (ErbB2, neu), HER3 (ErbB3), and HER4 (ErbB4)]. Overexpression, mutation, or aberrant activity of these receptors has been implicated in various types of cancer (Feldinger K, Breast Cancer (Dove Med Press), 2015, 7, 147).

First-Generation Inhibitors

Erlotinib and Gefitinib are small molecule inhibitors of the EGFR/HER-1 (human epidermal growth factor receptor) tyrosine kinase. Erlotinib and Gefitinib were developed as reversible and highly specific small-molecule tyrosine kinase inhibitors that competitively block the binding of adenosine triphosphate to its binding site in the tyrosine kinase domain of EGFR, thereby inhibiting autophosphorylation and blocking downstream signaling (Cataldo V D, N Engl J Med, 2011, 364, 947).

Second-Generation Inhibitors

Afatinib is an oral tyrosine kinase inhibitor (TKI) approved for the first-line treatment of patients with NSCLC whose tumors are driven by activating mutations of genes coding for epidermal growth factor receptor (EGFR). Afatinib is also an inhibitor of a specific EGFR mutation (T790M) that causes resistance to first-generation EGFR-targeted TKIs in about half of patients receiving those drugs. (Engle J A, Am J Health Syst Pharm 2014, 71 (22), 1933).

Neratinib, a pan-HER inhibitor, irreversible tyrosine kinase inhibitor binds and inhibits the tyrosine kinase activity of epidermal growth factor receptors, EGFR (or HER1), HER2 and HER4, which leads to reduced phosphorylation and activation of downstream signaling pathways. Neratinib has been shown to be effective against HER2-overexpressing or mutant tumors in vitro and in vivo. Neratinib is currently being investigated in various clinical trials in breast cancers and other solid tumors, including those with HER2 mutation (Feldinger K, Breast Cancer (Dove Med Press), 2015, 7, 147).

Dacomitinib is an irreversible inhibitor of EGFR, HER2, and HER4. In preclinical cell lines and xenograft studies, dacomitinib demonstrated activities against both activating EGFR mutations and EGFR T790M (Liao B C, Curr Opin Oncol. 2015, 27(2), 94).

Third-Generation Inhibitors

The third-generation EGFR-TKIs were designed to inhibit EGFR T790M while sparing wild-type EGFR.

AZD9291 (AstraZeneca, Macclesfield, UK), a mono-anilino-pyrimidine compound, is an irreversible mutant selective EGFR-TKI. This drug is structurally different from the first and second-generation EGFR-TKIs. In preclinical studies, it potently inhibited phosphorylation of EGFR in cell lines with activating EGFR mutations (EGFR den and EGFR L858R) and EGFR T790M. AZD9291 also caused profound and sustained tumor regression in tumor xenograft and transgenic mouse models harboring activating EGFR mutations and EGFR T790M. AZD9291 was less potent in inhibiting phosphorylation of wild-type EGFR cell lines (Liao B C, Curr Opin Oncol. 2015, 27(2), 94).

Rociletinib (CO-1686) (Clovis Oncology, Boulder, Colo), a 2,4-disubstituted pyrimidine molecule, is an irreversible mutant selective EGFR-TKI. In preclinical studies, CO-1686 led to tumor regression in cell-lines, xenograft models, and transgenic mouse models harboring activating EGFR mutations and EGFR T790M (Walter A O, Cancer Discov, 2013, 3(12), 1404).

HM61713 (Hanmi Pharmaceutical Company Ltd, Seoul, South Korea) is an orally administered, selective inhibitor for activating EGFR mutations and EGFR T790M. It has low activity against wild-type EGFR (Steuer C E, Cancer. 2015, 121(8), E1).

It has now been found, and this constitutes the basis of the present invention, that the compounds of the present invention have surprising and advantageous properties.

In particular, the compounds of the present invention have surprisingly been found to effectively and selectively inhibit the Ras-Sos interaction without significantly targeting the EGFR receptor and may therefore be used for the treatment or prophylaxis of hyper-proliferative disorders, in particular cancer.

### DESCRIPTION OF THE INVENTION

In accordance with a first aspect, the present invention covers compounds of general formula (I):

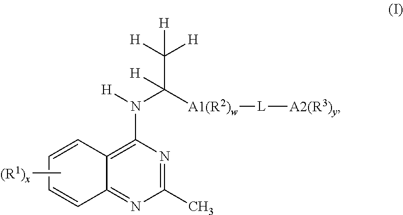

in which
R¹ stands for
   a substituent independently selected from: a hydrogen atom, a halogen atom, a hydroxy, cyano, nitro, C₁-C₆-alkylsulfanyl or an amino group —NR^{a}R^{b},
      wherein R^{a} and R^{b} are selected independently from a hydrogen atom or a C₁-C₆-alkyl,
   a substituent selected from: a C₁-C₆-alkyl, C₁-C₆-alkoxy-, C₂-C₆-alkenyl, C₂-C₆-alkynyl, C₃-C₈-cycloalkyl, C₄-C₈-cycloalkenyl, 4- to 7-membered heterocycloalkyl, 5- to 10-membered heterocycloalkenyl, heterospirocycloalkyl, fused heterocycloalkyl, bridged heterocycloalkyl, phenyl, heteroaryl, C₁-C₆-haloalkyl,
   —C(═O)OH, —C(═O)OR^{c}, and wherein R^{c} stands for C₁-C₆-alkyl, C₃-C₆-alkenyl, C₃-C₆-alkynyl, C₃-C₈-cycloalkyl or C₄-C₈-cycloalkenyl,
   —N═S(═O)(R^{d})R^{e}, and wherein R^{d} and R^{e} are selected independently from hydrogen, —C₁-C₆-alkyl, C₂-C₆-alkenyl, C₂-C₆-alkynyl, C₃-C₈-cycloalkyl or C₄-C₈-cycloalkenyl,
   —NH—C(O)—C₁-C₆-alkyl, —NH—C(O)—NR^{a}R^{b}, wherein R^{a} and R^{b} are selected independently from a hydrogen atom or a C₁-C₆-alkyl, —NH—(CH₂)ₖ—NH—C(O)—C₁-C₆-alkyl, wherein k is 1 or 2, —NH—(CH₂)ₗ—R^{f}, wherein
      l is 0, 1 or 2 and R^{f} stands for a 4- to 7-membered heterocycloalkyl, heteroaryl, C₁-C₆-alkylsulfonyl,
      whereby in all foregoing definitions the C₁-C₆-alkyl-C₁-C₆-alkoxy-, the 4- to 7-membered heterocycloalkyl and the heteroaryl can be optionally substituted, one, two or three times, identically or differently, with:
         a halogen atom, or a group selected from hydroxy, oxo (═O), a cyano, nitro, C₁-C₆-alkyl, C₂-C₆-alkenyl, C₂-C₆-alkynyl, C₃-C₈-cycloalkyl, 4- to 7-membered heterocycloalkyl, C₁-C₆-alkoxy, C₁-C₆-haloalkyl, C₁-C₆-haloalkoxy-, C₁-C₆-alkylsulfonyl, phenyl, benzyl-, heteroaryl, —(CH₂)-heteroaryl-, C₃-C₈-cycloalkoxy-, phenyloxy-, heteroaryloxy-, —NH—C(O)—C₁-C₆-alkyl or an amino group —NR^{a}R^{b}, wherein R^{a} and R^{b} are selected independently from a hydrogen atom or a C₁-C₆-alkyl, or
   a substituent

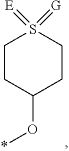

wherein E and G each stands for an electron pair, or one of E and G stands for an electron pair and the other for an oxygen atom or a group ═NH or ═N—C₁-C₄-alkyl, or one of E and G stands for an oxygen atom and the other one for a group ═NH or ═N—C₁-C₄-alkyl, or E and G each stands for an oxygen atom or each stands for a group ═NH or ═N—C₁-C₄-alkyl, or
a substituent —O—(CH₂)_{z}-phenyl, —O—(CH₂)_{z} C₄-C₇-heterocycloalkyl, —O—(CH₂)_{z}-heteroaryl, whereby
   z is 0, 1 or 2, and the phenyl, heterocycloalkyl and heteroaryl can optionally be substituted with a group selected from hydroxy, heterocycloalkyl or heterocycloalkenyl, which both can be substituted with a methyl- and/or oxo-group,
or a substituent selected from the group of

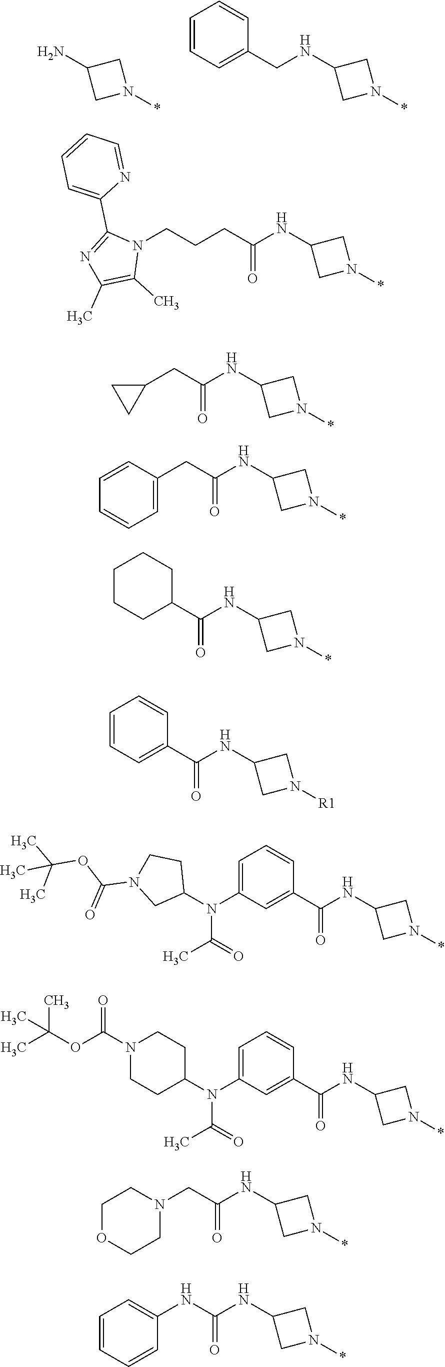

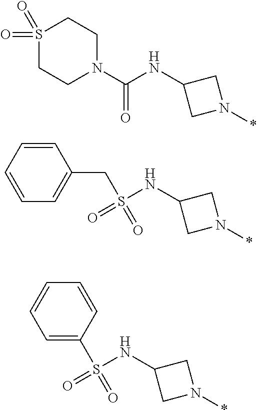 [SMILES: *N1CC(NC(=O)N2CCS(=O)(=O)CC2)C1 ; *N1CC(NS(=O)(=O)Cc2ccccc2)C1 ; *N1CC(NS(=O)(=O)c2ccccc2)C1]

and wherein x is 1, 2 or 3,
A1 stands for
   a C₄ to C₁₂ carbocyclic, heterocyclic, optionally bicyclic, optionally aromatic or optionally heteroaromatic ring system, wherein in a bicyclic aromatic or heteroaromatic ring system one or two double bonds can be hydrogenated,
R² stands for
   a hydrogen atom, a hydroxy group, oxo (═O), a halogen atom, a cyano group, a substituent selected from: a C₁-C₆-alkyl, C₁-C₆-alkoxy-, C₂-C₆-alkenyl, C₂-C₆-alkynyl, C₃-C₈-cycloalkyl, C₄-C₈-cycloalkenyl, 4- to 7-membered heterocycloalkyl, —O—CH₂-4- to 7-membered heterocycloalkyl, 5- to 10-membered heterocycloalkenyl, heterospirocycloalkyl, fused heterocycloalkyl, bridged heterocycloalkyl, phenyl, heteroaryl, C₁-C₆-haloalkyl, C₁-C₆-alkylsulfonyl,
   —NR^{a}R^{b}, wherein R^{a} and R^{b} are selected independently from a hydrogen atom or a C₁-C₆-alkyl,
   —C(O)—NR^{a}R^{b}, wherein R^{a} and R^{b} are selected independently from a hydrogen atom or a C₁-C₆-alkyl, —C(O)—O—R^{g}, wherein R^{g} is a hydrogen atom or a C₁-C₆-alkyl, —O—R^{h}, wherein R^{h} is a C₁-C₆-alkyl or —CH₂—NR^{a}R^{b}, wherein R^{a} and R^{b} are selected independently from a hydrogen atom or a C₁-C₆-alkyl,
and w is 1 or 2,
and wherein
A2(R³)_{y} stands either for a hydrogen atom or
A2 has the same meanings as the substituent A1 and
R³ stands for
   a hydrogen atom, a halogen atom, a hydroxy, oxo, cyano, nitro group, a substituent selected from a C₁-C₆-alkyl, C₂-C₆-alkenyl, C₂-C₆-alkynyl, C₃-C₈-cycloalkyl, C₄-C₈-cycloalkenyl, C₇-C₈-cycloalkynyl, 4- to 7-membered heterocycloalkyl, 5- to 10-membered heterocycloalkenyl, phenyl, heteroaryl, C₁-C₆-haloalkyl,
      which substituent is optionally substituted, one, two or three times, identically or differently, with a substituent selected from:
         a halogen atom, or a group selected from hydroxy, oxo (═O), cyano, C₁-C₆-alkyl, C₁-C₆-alkoxy, C₁-C₆-haloalkyl, phenyl, —C(O)NRⁱR^{j}, wherein
         Rⁱ and R^{j} are selected independently from a hydrogen atom or a C₁-C₆-alkyl, heteroaryl,
      or with amino —NR^{k}R^{l}, wherein R^{k} and R^{l} are selected independently from
         a hydrogen atom, a substituent selected from a C₁-C₆-alkyl, C₂-C₆-alkenyl, C₂-C₆-alkynyl, C₃-C₈-cycloalkyl, C₁-C₆-alkylsulfonyl, phenyl, heteroaryl, 4- to 7-membered heterocycloalkyl, which are optionally substituted
         one, two or three times, identically or differently, with a substituent selected from C₁-C₆-haloalkyl, hydroxyl, oxo (═O), phenyl, cyano, C₁-C₆-alkoxy, heteroaryl, wherein
         the heteroaryl can optionally be substituted with a methyl group, or —CH₂—C(O)—R^{m}, wherein
         R^{m} is a bicyclic heteroaryl, which can be partially hydrogenated, a C₁-C₆-alkoxy or a group —NRⁿR^{o}, in which
         Rⁿ and R^{o} are selected independently from hydrogen, C₁-C₆-alkyl, phenyl, wherein the C₁-C₆-alkyl can optionally be substituted with a C₁-C₆-alkoxy or a phenyl, or
         —NRⁿR^{o} stands for a 4- to 7-membered azacycloalkyl, bound via the nitrogen atom to the rest of the molecule and which optionally contains one more heteroatom selected from nitrogen and oxygen;
         —C(═O)R^{p}, wherein R^{p} is selected from
         the group of a C₁-C₆-alkoxy, a C₁-C₆-alkyl, which is optionally substituted, one, two or three times, identically or differently, with a substituent selected from hydroxyl or C₁-C₆-alkoxy, a mono- or bicyclic heteroaryl, a 4- to 7-membered heterocycloalkyl or R^{p} is a group —CH₂—NR^{q}R^{r}; wherein R^{q} and R^{r} are selected independently from hydrogen, phenyl or a C₁-C₆-alkyl, which may optionally be substituted up to threefold with fluorine,
      NR^{s}R^{t} is
         a 4- to 7-membered azacycloalkyl, bound via the nitrogen atom to the rest of the molecule, or a 6- to 10-membered azaspirocycloalkyl, which both may contain up to 2 further heteroatoms selected from nitrogen and oxygen and which both are optionally substituted one, two or three times, identically or differently, with a substituent selected from: hydroxy, oxo (═O), C₁-C₆-alkyl, C₁-C₆-hydroxyalkyl, —C(═O)OR^{u}, wherein R^{u} is a C₁-C₆-alkyl, halogen, —N(C₁-C₆-alkyl)₂, —CH₂—N(C₁-C₆-alkyl)₂, —C(O)NR^{a}R^{b}, wherein R^{a} and R^{b} are selected independently from a hydrogen atom or a C₁-C₆-alkyl,
   —C(═O)R^{v}, —C(═O)NH₂, —C(═O)N(H)R^{v}, —C(═O)N(R^{v})R^{w}, —C(═O)OR^{v}, wherein
      R^{v} and R^{w} represent, independently from each other, a group selected from hydrogen, C₁-C₄-alkyl, C₁-C₄-haloalkyl, phenyl, or a group —(CH₂)₂—NR^{x}R^{y}, wherein R^{x} and R^{y} independently from each other stand for hydrogen, a C₁-C₄-alkyl or a group —(CH₂)₂N(CH₃)₂;
   —NH₂, —NHR^{z}, —N(R^{z})R^{za}, —N(H)C(═O)R^{z}, —N(H)C(═O)OR^{z}, —N(H)S(═O)₂R^{z}, 4- to 7-membered heterocycloalkyl, heteroaryl, heterospirocycloalkyl, fused heterocycloalkyl, bridged heterocycloalkyl, wherein
      R^{z} and R^{za} represent, independently from each other, a group selected from C₁-C₄-alkyl, C₁-C₄-haloalkyl and phenyl,
   C₁-C₆-alkoxy-, C₁-C₆-haloalkoxy-, —O—(CH₂)ₛ—C₃-C₈-cycloalkyl, —O—(CH₂)ₛ-phenyl, —O—(CH₂)ₛ-heterocycloalkyl, —O—(CH₂)ₛ-heteroaryl, s is 0, 1, 2 or 3,
   —S(═O)₂R^{z}, —S(═O)₂NH₂, —S(═O)₂NHR^{z}, —S(═O)₂N(R^{z})R^{za}, wherein R^{z} and R^{za} represent, independently from each other, a group selected from C₁-C₄-alkyl, C₁-C₄-haloalkyl and phenyl,
wherein y is 1, 2 or 3, and
L stands either for a bond or for —O—(CH₂)ₖ, wherein k is 0, 1, 2 or 3, or a group —CH═CH—(CH₂)ₙ, wherein n is 0, 1 or 2,
or a tautomer, an N-oxide, a hydrate, a solvate, or a salt thereof, or a mixture of same.

### Definitions

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two or three identical or different substituents, particularly with one substituent.

As used herein, an oxo substituent represents an oxygen atom, which is bound to a carbon atom or to a sulfur atom via a double bond.

The term “ring substituent” means a substituent attached to an aromatic or nonaromatic ring which replaces an available hydrogen atom on the ring.

Should a composite substituent be composed of more than one parts, e.g. (C₁-C₄-alkoxy)-(C₁-C₄-alkyl)-, it is possible for the position of a given part to be at any suitable position of said composite substituent, i.e. the C₁-C₄-alkoxy part can be attached to any carbon atom of the C₁-C₄-alkyl part of said (C₁-C₄-alkoxy)-(C₁-C₄-alkyl)- group. A hyphen at the beginning or at the end of such a composite substituent indicates the point of attachment of said composite substituent to the rest of the molecule. Should a ring, comprising carbon atoms and optionally one or more heteroatoms, such as nitrogen, oxygen or sulfur atoms for example, be substituted with a substituent, it is possible for said substituent to be bound at any suitable position of said ring, be it bound to a suitable carbon atom and/or to a suitable heteroatom.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen atom” means a fluorine, chlorine, bromine or iodine atom, particularly a fluorine, chlorine or bromine atom.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group.

The term “C₁-C₆-hydroxyalkyl” means a linear or branched, saturated, monovalent hydrocarbon group in which the term “C₁-C₆-alkyl” is defined supra, and in which 1, 2 or 3 hydrogen atoms are replaced with a hydroxy group, e.g. a hydroxymethyl, 1-hydroxyethyl, 2-hydroxyethyl, 1,2-dihydroxyethyl, 3-hydroxypropyl, 2-hydroxypropyl, 1-hydroxypropyl, 1-hydroxypropan-2-yl, 2-hydroxypropan-2-yl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl group.

The term “C₁-C₆-alkylsulfanyl” means a linear or branched, saturated, monovalent group of formula (C₁-C₆-alkyl)-S—, in which the term “C₁-C₆-alkyl” is as defined supra, e.g. a methylsulfanyl, ethylsulfanyl, propylsulfanyl, isopropylsulfanyl, butylsulfanyl, sec-butylsulfanyl, isobutylsulfanyl, tert-butylsulfanyl, pentylsulfanyl, isopentylsulfanyl, hexylsulfanyl group.

The term “C₁-C₆-alkylsulfonyl” means a linear or branched, saturated, monovalent group of formula (C₁-C₆-alkyl)-SO₂—, in which the term “C₁-C₆-alkyl” is as defined supra, e.g. a methylsulfonyl, ethylsulfonyl, propylsulfonyl, isopropylsulfonyl, butylsulfonyl, sec-butylsulfonyl, isobutylsulfonyl, tert-butylsulfonyl, pentylsulfonyl, isopentylsulfonyl, hexylsulfonyl group.

The term “C₁-C₆-alkoxy” means a linear or branched, saturated, monovalent group of formula (C₁-C₆-alkyl)-O—, in which the term “C₁-C₆-alkyl” is as defined supra, e.g. a methoxy, ethoxy, n-propoxy, isopropoxy, n-butoxy, sec-butoxy, isobutoxy, tert-butoxy, pentyloxy, isopentyloxy or n-hexyloxy group, or an isomer thereof.

The term “C₂-C₆-alkenyl” means a linear or branched, monovalent hydrocarbon group, which contains one or two double bonds, and which has 2, 3, 4, 5 or 6 carbon atoms, particularly 2 or 3 carbon atoms (“C₂-C₃-alkenyl”), it being understood that in the case in which said alkenyl group contains more than one double bond, then it is possible for said double bonds to be isolated from, or conjugated with, each other. Said alkenyl group is, for example, an ethenyl (or “vinyl”), prop-2-en-1-yl (or “allyl”), prop-1-en-1-yl, but-3-enyl, but-2-enyl, but-1-enyl, pent-4-enyl, pent-3-enyl, pent-2-enyl, pent-1-enyl, hex-5-enyl, hex-4-enyl, hex-3-enyl, hex-2-enyl, hex-1-enyl, prop-1-en-2-yl (or “isopropenyl”), 2-methylprop-2-enyl, 1-methylprop-2-enyl, 2-methylprop-1-enyl, 1-methylprop-1-enyl, 3-methylbut-3-enyl, 2-methylbut-3-enyl, 1-methylbut-3-enyl, 3-methylbut-2-enyl, 2-methylbut-2-enyl, 1-methylbut-2-enyl, 3-methylbut-1-enyl, 2-methylbut-1-enyl, 1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, 3-methylpent-3-enyl, 2-methylpent-3-enyl, 1-methylpent-3-enyl, 4-methylpent-2-enyl, 3-methylpent-2-enyl, 2-methylpent-2-enyl, 1-methylpent-2-enyl, 4-methylpent-1-enyl, 3-methylpent-1-enyl, 2-methylpent-1-enyl, 1-methylpent-1-enyl, 3-ethylbut-3-enyl, 2-ethylbut-3-enyl, 1-ethylbut-3-enyl, 3-ethylbut-2-enyl, 2-ethylbut-2-enyl, 1-ethylbut-2-enyl, 3-ethylbut-1-enyl, 2-ethylbut-1-enyl, 1-ethylbut-1-enyl, 2-propylprop-2-enyl, 1-propylprop-2-enyl, 2-isopropylprop-2-enyl, 1-isopropylprop-2-enyl, 2-propylprop-1-enyl, 1-propylprop-1-enyl, 2-isopropylprop-1-enyl, 1-isopropylprop-1-enyl, 3,3-dimethylprop-1-enyl, 1-(1,1-dimethylethyl)ethenyl, buta-1,3-dienyl, penta-1,4-dienyl or hexa-1,5-dienyl group. Particularly, said group is vinyl or allyl.

The term “C₂-C₆-alkynyl” means a linear or branched, monovalent hydrocarbon group which contains one triple bond, and which contains 2, 3, 4, 5 or 6 carbon atoms, particularly 2 or 3 carbon atoms (“C₂-C₃-alkynyl”). Said C₂-C₆-alkynyl group is, for example, ethynyl, prop-1-ynyl, prop-2-ynyl (or “propargyl”), but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methyl-pent-4-ynyl, 2-methylpent-3-ynyl, 1-methylpent-3-ynyl, 4-methylpent-2-ynyl, 1-methyl-pent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or 3,3-dimethylbut-1-ynyl group. Particularly, said alkynyl group is ethynyl, prop-1-ynyl or prop-2-ynyl.

The term “C₃-C₈-cycloalkyl” means a saturated, monovalent, mono- or bicyclic hydrocarbon ring which contains 3, 4, 5, 6, 7 or 8 carbon atoms (“C₃-C₈-cycloalkyl”). Said C₃-C₈-cycloalkyl group is for example, a monocyclic hydrocarbon ring, e.g. a cyclopropyl, cyclobutyl, cyclopentyl, cyclohexyl, cycloheptyl or cyclooctyl group, or a bicyclic hydrocarbon ring, e.g. a bicyclo[4.2.0]octyl or octahydropentalenyl.

The term “C₄-C₈-cycloalkenyl” means a monovalent, mono- or bicyclic hydrocarbon ring which contains 4, 5, 6, 7 or 8 carbon atoms and one double bond. Particularly, said ring contains 4, 5 or 6 carbon atoms (“C₄-C₆-cycloalkenyl”). Said C₄-C₈-cycloalkenyl group is for example, a monocyclic hydrocarbon ring, e.g. a cyclobutenyl, cyclopentenyl, cyclohexenyl, cycloheptenyl or cyclooctenyl group, or a bicyclic hydrocarbon ring, e.g. a bicyclo[2.2.1]hept-2-enyl or bicyclo[2.2.2]oct-2-enyl.

The term “C₃-C₈-cycloalkoxy” means a saturated, monovalent, mono- or bicyclic group of formula (C₃-C₈-cycloalkyl)-O—, which contains 3, 4, 5, 6, 7 or 8 carbon atoms, in which the term “C₃-C₈-cycloalkyl” is defined supra, e.g. a cyclopropyloxy, cyclobutyloxy, cyclopentyloxy, cyclohexyloxy, cycloheptyloxy or cyclooctyloxy group.

The term “spirocycloalkyl” means a saturated, monovalent bicyclic hydrocarbon group in which the two rings share one common ring carbon atom, and wherein said bicyclic hydrocarbon group contains 5, 6, 7, 8, 9, 10 or 11 carbon atoms, it being possible for said spirocycloalkyl group to be attached to the rest of the molecule via any one of the carbon atoms except the spiro carbon atom. Said spirocycloalkyl group is, for example, spiro[2.2]pentyl, spiro[2.3]hexyl, spiro[2.4]heptyl, spiro[2.5]octyl, spiro[2.6]nonyl, spiro[3.3]heptyl, spiro[3.4]octyl, spiro[3.5]nonyl, spiro[3.6]decyl, spiro[4.4]nonyl, spiro[4.5]decyl, spiro[4.6]undecyl or spiro[5.5]undecyl.

The terms “4- to 7-membered heterocycloalkyl” means a monocyclic, saturated heterocycle with 4, 5, 6 or 7 ring atoms in total, which contains one or two identical or different ring heteroatoms from the series N, O and S, it being possible for said heterocycloalkyl group to be attached to the rest of the molecule via any one of the carbon atoms or, if present, a nitrogen atom.

Said heterocycloalkyl group, without being limited thereto, can be a 4-membered ring, such as azetidinyl, oxetanyl or thietanyl, for example; or a 5-membered ring, such as tetrahydrofuranyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, for example; or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, for example, or a 7-membered ring, such as azepanyl, 1,4-diazepanyl or 1,4-oxazepanyl, for example.

Particularly, “4- to 6-membered heterocycloalkyl” means a 4- to 6-membered heterocycloalkyl as defined supra containing one ring nitrogen atom and optionally one further ring heteroatom from the series: N, O, S. More particularly, “5- or 6-membered heterocycloalkyl” means a monocyclic, saturated heterocycle with 5 or 6 ring atoms in total, containing one ring nitrogen atom and optionally one further ring heteroatom from the series: N, O.

The term “4- to 7-memebered azacycloalkyl” means a monocyclic saturated heterocycly with 4, 5, 6 or 7 ring atoms in total which is attached to the rest of the molecule via the nitrogen atom and which optionally contains one more heteroatom selected from nitrogen and oxygen.

Said 4- to 7-membered azacycloalkyl group, without being limited thereto, can be a 4-membered ring, such as azetidin-1-yl, for example; or a 5-membered ring, such as pyrrolidin-1-yl, imidazolidin-1-yl, pyrazolidin-1-yl, 1,2-oxazolidin-2-yl or 1,3-oxazolidin-3-yl, for example; or a 6-membered ring, such as piperidin-1-yl, morpholin-4-yl, piperazin-1-yl or 1,2-oxazinan-2-yl, for example, or a 7-membered ring, such as azepan-1-yl, 1,4-diazepan-1-yl or 1,4-oxazepan-4-yl, for example.

The term “5- to 10-membered heterocycloalkenyl” means a monocyclic, unsaturated, non-aromatic heterocycle with 5, 6, 7, 8, 9 or 10 ring atoms in total, which contains one or two double bonds and one or two identical or different ring heteroatoms from the series: N, O, S; it being possible for said heterocycloalkenyl group to be attached to the rest of the molecule via any one of the carbon atoms or, if present, a nitrogen atom.

Said heterocycloalkenyl group is, for example, 4H-pyranyl, 2H-pyranyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl or 4H-[1,4]thiazinyl.

The term “heterospirocycloalkyl” means a bicyclic, saturated heterocycle with 6, 7, 8, 9, 10 or 11 ring atoms in total, in which the two rings share one common ring carbon atom, which “heterospirocycloalkyl” contains one, two or three identical or different ring heteroatoms from the series: N, O, S; it being possible for said heterospirocycloalkyl group to be attached to the rest of the molecule via any one of the carbon atoms, except the spiro carbon atom, or, if present, a nitrogen atom.

Said heterospirocycloalkyl group is, for example, azaspiro[2.3]hexyl, azaspiro[3.3]heptyl, oxaazaspiro[3.3]heptyl, thiaazaspiro[3.3]heptyl, oxaspiro[3.3]heptyl, oxazaspiro[5.3]nonyl, oxazaspiro[4.3]octyl, azaspiro[4,5]decyl, oxazaspiro[5.5]undecyl, diazaspiro[3.3]heptyl, thiazaspiro[3.3]heptyl, thiazaspiro[4.3]octyl, azaspiro[5.5]undecyl, or one of the further homologous scaffolds such as spiro[3.4]-, spiro[4.4]-, spiro[2.4]-, spiro[2.5]-, spiro[2.6]-, spiro[3.5]-, spiro[3.6]-, spiro[4.5]- and spiro[4.6]-.

The term “6- to 10-membered azaspirocycloalkyl” means a bicyclic, saturated heterocycle with 6, 7, 8, 9 or 10 ring atoms in total, in which the two rings share one common ring carbon atom and which is bound to the rest of the molecule via the nitrogen atom and which azaspirocycloalkyl may contain up to 2 further heteroatoms selected from nitrogen and oxygen.

Said azaspirocycloalkyl is for example, azaspiro[2.3]hexyl, azaspiro[3.3]heptyl, oxaazaspiro[3.3]heptyl, oxazaspiro[5.3]nonyl, oxazaspiro[4.3]octyl, azaspiro[4,5]decyl, oxazaspiro[5.5]undecyl, diazaspiro[3.3]heptyl, triazaspiro[3.4]octyl or one of the further homologous scaffolds such as spiro[3.4]-, spiro[4.4]-, spiro[2.4]-, spiro[2.5]-, spiro[2.6]-, spiro[3.5]-, spiro[3.6]- and spiro[4.5]-, whereby these azaspirocycloalkyl groups are always bound via the nitrogen atom to the rest of the molecule. Of these groups preference is given to 2-oxa-6-azaspiro[3.3]hept-6-yl and 2,5,7-triazaspiro[3.4]octan-2-yl.

The term “fused heterocycloalkyl” means a bicyclic, saturated heterocycle with 6, 7, 8, 9 or 10 ring atoms in total, in which the two rings share two adjacent ring atoms, which “fused heterocycloalkyl” contains one or two identical or different ring heteroatoms from the series: N, O, S; it being possible for said fused heterocycloalkyl group to be attached to the rest of the molecule via any one of the carbon atoms or, if present, a nitrogen atom.

Said fused heterocycloalkyl group is, for example, azabicyclo[3.3.0]octyl, azabicyclo[4.3.0]nonyl, diazabicyclo[4.3.0]nonyl, oxazabicyclo[4.3.0]nonyl, thiazabicyclo[4.3.0]nonyl or azabicyclo[4.4.0]decyl.

The term “bridged heterocycloalkyl” means a bicyclic, saturated heterocycle with 7, 8, 9 or 10 ring atoms in total, in which the two rings share two common ring atoms which are not adjacent, which “bridged heterocycloalkyl” contains one or two identical or different ring heteroatoms from the series: N, O, S; it being possible for said bridged heterocycloalkyl group to be attached to the rest of the molecule via any one of the carbon atoms, except the spiro carbon atom, or, if present, a nitrogen atom.

Said bridged heterocycloalkyl group is, for example, azabicyclo[2.2.1]heptyl, oxazabicyclo[2.2.1]heptyl, thiazabicyclo[2.2.1]heptyl, diazabicyclo[2.2.1]heptyl, azabicyclo-[2.2.2]octyl, diazabicyclo[2.2.2]octyl, oxazabicyclo[2.2.2]octyl, thiazabicyclo[2.2.2]octyl, azabicyclo[3.2.1]octyl, diazabicyclo[3.2.1]octyl, oxazabicyclo[3.2.1]octyl, thiazabicyclo[3.2.1]octyl, azabicyclo[3.3.1]nonyl, diazabicyclo[3.3.1]nonyl, oxazabicyclo[3.3.1]nonyl, thiazabicyclo[3.3.1]nonyl, azabicyclo[4.2.1]nonyl, diazabicyclo[4.2.1]nonyl, oxazabicyclo[4.2.1]nonyl, thiazabicyclo[4.2.1]nonyl, azabicyclo[3.3.2]decyl, diazabicyclo[3.3.2]decyl, oxazabicyclo[3.3.2]decyl, thiazabicyclo[3.3.2]decyl or azabicyclo[4.2.2]decyl.

The term “heteroaryl” means a monovalent, monocyclic, bicyclic or tricyclic aromatic ring having 5, 6, 8, 9, 10, 11, 12, 13 or 14 ring atoms (a “5- to 14-membered heteroaryl” group), particularly 5, 6, 9 or 10 ring atoms, which contains at least one ring heteroatom and optionally one, two or three further ring heteroatoms from the series: N, O and/or S, and which is bound via a ring carbon atom or optionally via a ring nitrogen atom (if allowed by valency).

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or a tricyclic heteroaryl group, such as, for example, carbazolyl, acridinyl or phenazinyl; a 8-membered heteroaryl group, such as for example 6,7-dihydro-5H-pyrrolo[1,2-a]imidazolyl or a 9-membered heteroaryl group, such as, for example, benzofuranyl, benzothienyl, benzoxazolyl, benzisoxazolyl, benzimidazolyl, benzothiazolyl, benzothiadiazolyl, benzotriazolyl, indazolyl, indolyl, isoindolyl, indolizinyl, thienopyridinyl, 1H-pyrrolo[2,3-b]pyridinyl or purinyl; or a 10-membered heteroaryl group, such as, for example, quinolinyl, quinazolinyl, isoquinolinyl, cinnolinyl, phthalazinyl, quinoxalinyl or pteridinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

A C4 to C12 carbocyclic, heterocyclic, optionally bicyclic, optionally aromatic or optionally heteroaromatic ring system, wherein in a bicyclic, aromatic or heteroaromatic ring system one or two double bonds can be hydrogenated is selected from the group of the substituents phenyl, naphthyl, 1,2,3,4-tetrahydronaphthyl, 1,3-benzodioxolyl, quinolinyl, isoquinolinyl, 2,3-dihydro-1,4-benzodioxinyl, imidazo[1,2-a]pyridinyl, furanyl, thienyl, pyridinyl, 2H-1,4-benzoxazinyl-3(4H)-one, 2,1,3-benzothiadiazolyl, 1-benzofuranyl, 1-benzothienyl, 1H indazolyl, 1H-indolyl, 1H-benzimidazolyl, 1,3-benzothiazolyl, thieno[2,3-b]pyridinyl, thieno[2,3-c]pyridinyl, thieno[3,2-c]pyridinyl, pyrimidinyl, 1H-pyrazolyl, 6,7-dihydro-5H-pyrrolo[1,2-a]imidazolyl, 1,2-oxazolyl, 1H-imidazolyl, 1,3,4-oxadiazolyl, 1H-tetrazolyl, 1H-pyrrolyl, 1H-pyrrolo[2,3-b]pyridinyl or 3,4-dihydro-2H-1,4-benzoxazinyl.

Particularly, the heteroaryl group is a quinolinyl, isoquinolinyl, imidazo[1,2-a]pyridinyl, furanyl, thienyl, pyridinyl, 2,1,3-benzothiadiazolyl, 1-benzofuranyl, 1-benzothiophenyl, 1H-indazolyl, 1H-indolyl, 1H-benzimidazolyl, 1,3-benzothiazolyl, thieno[2,3-b]pyridinyl, thieno[2,3-c]pyridinyl, thieno[3,2-c]pyridinyl, pyrimidinyl, 1H-pyrazolyl, 6,7-dihydro-5H-pyrrolo[1,2-a]imidazolyl, 1,2-oxazolyl, 1H-imidazolyl, 1,3,4-oxadiazolyl, 1H-tetrazolyl, 1H-pyrrolyl, 1H pyrrolo[2,3-b]pyridinyl or 3,4-dihydro-2H-1,4-benzoxazinyl group.

In composite substituents such as C₁-C₆-haloalkyl, C₁-C₆-haloalkyl, C₁-C₆-haloalkoxy, —(CH₂)-heteroaryl, heteroaryloxy, —O—(CH₂)ₓ-heteroaryl, —O—(CH₂)_{z}-heteroaryl, O—(CH₂)-4- to 7-membered heterocycloalkyl, bicyclic heteroaryl, C₁-C₆-hydroxyalkyl, —O—(CH₂)ₓ—C₃-C₈-cycloalkyl, O—(CH₂)ₓ-phenyl, —O—(CH₂)ₓ-heterocyclyl and C₃-C₈-cycloalkyloxy the definition of the residue to which the further substituent is attached is the same as given for the residues which do not bear a further substituent, e.g. in C₁-C₆-haloalkyl the C₁-C₆-alkyl has the same meanings as given for the C₁-C₆-alkyl earlier.

The term “C₁-C₆”, as used in the present text, e.g. in the context of the definition of “C₁-C₆-alkyl”, “C₁-C₆-haloalkyl”, “C₁-C₆-hydroxyalkyl”, “C₁-C₆-alkoxy” or “C₁-C₆-haloalkoxy” means an alkyl group having a finite number of carbon atoms of 1 to 6, i.e. 1, 2, 3, 4, 5 or 6 carbon atoms.

Further, as used herein, the term “C₃-C₈°”, as used in the present text, e.g. in the context of the definition of “C₃-C₈-cycloalkyl”, means a cycloalkyl group having a finite number of carbon atoms of 3 to 8, i.e. 3, 4, 5, 6, 7 or 8 carbon atoms.

When a range of values is given, said range encompasses each value and sub-range within said range.

For example:
“C₁-C₆” encompasses C₁, C₂, C₃, C₄, C₅, C₆, C₁-C₆, C₁-C₅, C₁-C₄, C₁-C₃, C₁-C₂, C₂- C₆, C₂-C₅, C₂-C₄, C₂-C₃, C₃-C₆, C₃-C₅, C₃-C₄, C₄-C₆, C₄-C₆, and C₅-C₆;
“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;
“C₃-C₁₀” encompasses C₃, C₄, C₅, C₆, C₇, C₈, C₉, C₁₀, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₃-C₈” encompasses C₃, C₄, C₅, C₆, C₇, C₈, C₃-C₈, C₃-C₇, C₃-C₆, C₃-C₅, C₃-C₄, C₄- C₈, C₄-C₇, C₄-C₆, C₄-C₈, C₅-C₈, C₅-C₇, C₅-C₆, C₆-C₈, C₆-C₇ and C₇-C₈;
“C₃-C₆” encompasses C₃, C₄, C₅, C₆, C₃-C₆, C₃-C₅, C₃-C₄, C₄-C₆, C₄-C₅, and C₅-C₆;
“C₄-C₈” encompasses C₄, C₅, C₆, C₇, C₈, C₄-C₈, C₄-C₇, C₄-C₆, C₄-C₈, C₅-C₈, C₅-C₇, C₅-C₆, C₆-C₈, C₆-C₇ and C₇-C₈;
“C₄-C₇” encompasses C₄, C₅, C₆, C₇, C₄-C₇, C₄-C₆, C₄-C₈, C₅-C₇, C₅-C₆ and C₆-C₇;
“C₄-C₆” encompasses C₄, C₅, C₆, C₄-C₆, C₄-C₈ and C₅-C₆;
“C₅-C₁₀” encompasses C₅, C₆, C₇, C₈, C₉, C₁₀, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₆-C₁₀” encompasses C₆, C₇, C₈, C₉, C₁₀, C₆-C₁₀, C₆-C₉, C₆-C₈, C₆-C₇, C₇-C₁₀, C₇-C₉, C₇-C₈, C₈-C₁₀, C₈-C₉ and C₉-C₁₀.

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)-sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butylphenyl)sulfonyl]oxy and [(4-methoxyphenyl)sulfonyl]oxy.

It is possible for the compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes that constitute such a compound.

The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ¹²⁹I and ¹³¹I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴O, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, MA, USA; and CombiPhos Catalysts, Inc., Princeton, NJ, USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect.

Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

In another embodiment the present invention concerns a deuterium-containing compound of general formula (I), in which one, two or three of the hydrogen atom(s) in either one or both of the methyl groups shown in general formula (I) is/are replaced with a deuterium atom. Also the hydrogen atom on the carbon atom between the nitrogen atom and the group A1 can be replaced with a deuterium atom either as the single replacement of a hydrogen by a deuterium or in addition to the beforementioned replacements in either one or both of the methyl groups shown in general formula (I).

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention contain at least one or optionally even more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Preferred isomers are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S) isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, it is possible for the compounds of the present invention to exist as tautomers. For example, any compound of the present invention which contains an imidazopyridine moiety as a heteroaryl group for example can exist as a 1H tautomer, or a 3H tautomer, or even a mixture in any amount of the two tautomers, namely:

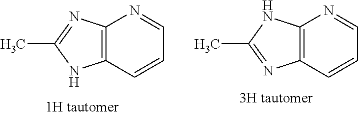

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quaternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown. Unless specified otherwise, suffixes to chemical names or structural formulae relating to salts, such as “hydrochloride”, “trifluoroacetate”, “sodium salt”, or “xHCl”, “xCF₃COOH”, “xNa⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

As used herein, the term “in vivo hydrolysable ester” means an in vivo hydrolysable ester of a compound of the present invention containing a carboxy or hydroxy group, for example, a pharmaceutically acceptable ester which is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexylcarbonyloxyethyl; 1,3-dioxolen-2-onylmethyl esters, e.g. 5-methyl-1,3-dioxolen-2-onylmethyl; and C₁-C₆-alkoxycarbonyloxyethyl esters, e.g. 1-methoxycarbonyloxyethyl, it being possible for said esters to be formed at any carboxy group in the compounds of the present invention.

An in vivo hydrolysable ester of a compound of the present invention containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive, but are converted (for example metabolically or hydrolytically) into compounds according to the invention during their residence time in the body.

In accordance with a second embodiment of the first aspect, the present invention covers compounds of general formula (I), supra, in which:
R¹ stands for
   a substituent independently selected from: a hydrogen atom, a halogen atom, a hydroxy, nitro, C₁-C₆-alkylsulfanyl or an amino group —NR^{a}R^{b},
      wherein R^{a} and R^{b} are selected independently from a hydrogen atom or a C₁-C₆-alkyl,
   a substituent selected from: a C₁-C₆-alkyl, C₁-C₆-alkoxy-, C₃-C₈-cycloalkyl, 4- to 7-membered heterocycloalkyl, heteroaryl,
   —C(═O)OH, —C(═O)OR^{c}, and wherein R^{o} stands for C₁-C₆-alkyl or C₃-C₈-cycloalkyl,
   —N═S(═O)(R^{d})R^{e}, and wherein R^{d} and R^{e} are selected independently from C₁-C₆-alkyl,
   —NH—C(O)—C₁-C₆-alkyl, —NH—C(O)—NR^{a}R^{b}, wherein R^{a} and R^{b} are selected independently from a hydrogen atom or a C₁-C₆-alkyl, —NH—(CH₂)k-NH—C(O)—C₁-C₆-alkyl, wherein k is 2, —NH—(CH₂)ₗ—R^{f}, wherein
   l is 0 or 2 and R^{f} stands for a 4- to 7-membered heterocycloalkyl or C₁-C₆-alkylsulfonyl,
   whereby in all foregoing definitions the C₁-C₆-alkyl-, C₁-C₆-alkoxy-, the 4- to 7 membered heterocycloalkyl and the heteroaryl can be optionally substituted, one or two or three times, identically or differently, with:
      a group selected from hydroxy, oxo (═O), C₁-C₆-alkyl, C₃-C₈-cycloalkyl, 4- to 7-membered heterocycloalkyl, C₁-C₆-alkoxy, C₁-C₆-alkylsulfonyl, benzyl, —(CH₂)-heteroaryl- or an amino group —NR^{a}R^{b}, wherein R^{a} and R^{b} are selected independently from C₁-C₆-alkyl, or
a substituent —O—(CH₂)_{z}-phenyl, whereby z is 0, 1 or 2, or
and wherein x is 1, 2 or 3,
A1 stands for
   a C₄ to C₁₂ carbocyclic, heterocyclic, optionally bicyclic, optionally aromatic or optionally heteroaromatic ring system, wherein in a bicyclic aromatic or heteroaromatic ring system one or two double bonds can be hydrogenated,
R² stands for
   a hydrogen atom, a hydroxy group, oxo (═O), a halogen atom, a cyano group, a substituent selected from: a C₁-C₆-alkyl, C₁-C₆-alkoxy-, C₂-C₆-alkenyl, C₃-C₈-cycloalkyl, 4- to 7-membered heterocycloalkyl, —O—CH₂-4- to 7-membered heterocycloalkyl, C₁-C₆-alkylsulfonyl,
   —C(O)—NR^{a}R^{b}, wherein R^{a} and R^{b} are both hydrogen atoms, —C(O)—O—R⁹, wherein R⁹ is a C₁-C₆-alkyl, or —CH₂—NR^{a}R^{b}, wherein R^{a} and R^{b} are both hydrogen atoms,
and w is 1 or 2,
and wherein
A2(R³)_{y} stands either for a hydrogen atom or
A2 has the same meanings as the substituent A1 and
R³ stands for
   a hydrogen atom, a halogen atom, a hydroxy, oxo, cyano, nitro group,
   a substituent selected from a C₁-C₆-alkyl, C₂-C₆-alkenyl, C₂-C₆-alkynyl, C₃-C₈-cycloalkyl, C₄-C₈-cycloalkenyl, C₇-C₈-cycloalkynyl, 4- to 7-membered heterocycloalkyl, 5- to 10-membered heterocycloalkenyl, phenyl, heteroaryl, C₁-C₆ haloalkyl,
      which substituent is optionally substituted, one, two or three times, identically or differently, with a substituent selected from:
         a halogen atom, or a group selected from hydroxy, oxo (═O), cyano, C₁-C₆-alkyl, C₁-C₆-alkoxy, C₁-C₆-haloalkyl, phenyl, —C(O)NRⁱR^{j}, wherein
         Rⁱ and R^{j} are selected independently from a hydrogen atom or a C₁-C₆-alkyl, heteroaryl,
      or with amino —NR^{k}R^{l}, wherein R^{k} and R^{l} are selected independently from
         a hydrogen atom, a substituent selected from a C₁-C₆-alkyl, C₂-C₆-alkenyl, C₂-C₆-alkynyl, C₃-C₈-cycloalkyl, C₁-C₆-alkylsulfonyl, phenyl, heteroaryl, 4- to 7-membered heterocycloalkyl, which are optionally substituted
         one, two or three times, identically or differently, with a substituent selected from C₁-C₆-haloalkyl, hydroxyl, oxo (═O), phenyl, cyano, C₁-C₆-alkoxy, heteroaryl, wherein
         the heteroaryl can optionally be substituted with a methyl group, or
         CH₂—C(O)—R^{m}, wherein
         R^{m} is a bicyclic heteroaryl, which can be partially hydrogenated, a C₁-C₆-alkoxy or a group —NRⁿR^{o}, in which
         Rⁿ and R^{o} are selected independently from hydrogen, C₁-C₆-alkyl, phenyl, wherein the C₁-C₆-alkyl can optionally be substituted with a C₁-C₆-alkoxy or a phenyl, or
         NRⁿR^{o} stands for a 4- to 7-membered azacycloalkyl, bound via the nitrogen atom to the rest of the molecule and which optionally contains one more heteroatom selected from nitrogen and oxygen;
         —C(═O)R^{p}, wherein R^{p} is selected from
         the group of a C₁-C₆-alkoxy, a C₁-C₆-alkyl, which is optionally substituted, one, two or three times, identically or differently, with a substituent selected from hydroxyl or C₁-C₆-alkoxy,
         a mono- or bicyclic heteroaryl, a 4- to 7-membered heterocycloalkyl or R^{p} is a group —CH₂—NR^{q}R^{r}; wherein R^{q} and R^{r} are selected independently from hydrogen, phenyl or a C₁-C₆-alkyl, which may optionally be substituted up to threefold with fluorine,
      NR^{s}R^{t} is
         a 4- to 7-membered azacycloalkyl, bound via the nitrogen atom to the rest of the molecule, or a 6- to 10-membered azaspirocycloalkyl, which both may contain up to 2 further heteroatoms selected from nitrogen and oxygen and which both are optionally substituted one, two or three times, identically or differently, with a substituent selected from: hydroxy, oxo (═O), C₁-C₆-alkyl, C₁-C₆-hydroxyalkyl, —C(═O)OR^{u}, wherein Ru is a C₁-C₆-alkyl, halogen, —N(C₁-C₆-alkyl)₂, —CH₂—N(C₁-C₆-alkyl)₂, —C(O)NR^{a}R^{b}, wherein R^{a} and R^{b} are selected independently from a hydrogen atom or a C₁-C₆-alkyl,
      —C(═O)R^{v}, —C(═O)NH₂, —C(═O)N(H)R^{v}, —C(═O)N(R^{v})R^{w}, —C(═O)OR^{v}, wherein
         R^{v} and R^{w} represent, independently from each other, a group selected from hydrogen, C₁-C₄-alkyl, C₁-C₄-haloalkyl, phenyl, or a group —(CH₂)₂—NR^{x}R^{y}, wherein R^{x} and R^{y} independently from each other stand for hydrogen, a C₁-C₄-alkyl or a group —(CH₂)₂N(CH₃)₂;
      —NH₂, —NHR^{z}, —N(R^{z})R^{za}, —N(H)C(═O)R^{z}, —N(H)C(═O)OR^{z}, —N(H)S(═O)₂R^{z}, 4- to 7-membered heterocycloalkyl, heteroaryl, heterospirocycloalkyl, fused heterocycloalkyl, bridged heterocycloalkyl, wherein
      R^{z} and R^{za} represent, independently from each other, a group selected from C₁-C₄-alkyl, C₁-C₄-haloalkyl and phenyl,
   C₁-C₆-alkoxy-, C₁-C₆-haloalkoxy-, —O—(CH₂)s-C₃-C₈-cycloalkyl, —O—(CH₂)ₛ-phenyl, —O—(CH₂)ₛ-heterocycloalkyl, —O—(CH₂)ₛ-heteroaryl, s is 0, 1, 2 or 3,
   —S(═O)₂R^{z}, —S(═O)₂NH₂, —S(═O)₂NHR^{z}, —S(═O)₂N(R^{z})R^{za}, wherein R^{z} and R^{za} represent, independently from each other, a group selected from C₁-C₄-alkyl, C₁-C₄-haloalkyl and phenyl,
wherein y is 1, 2 or 3, and
L stands either for a bond or for —O—(CH₂)ₖ, wherein k is 0, 1, 2 or 3, or a group —CH═CH—(CH₂)ₙ, wherein n is 0, 1 or 2,
or a tautomer, an N-oxide, a hydrate, a solvate, or a salt thereof, or a mixture of same.

In accordance with a third embodiment of the first aspect, the present invention covers compounds of general formula (I), supra, in which:
R¹ is selected from the list of the following substituents
H, *—OCH₃, *—OC₂H₅,

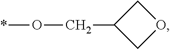 [SMILES: *OCC1COC1]

*—CH₂OH, *—C(O)OH, *—C(O)OCH₃, —Br, *—O—CH(CH₃)₂, *—O—(CH₂)₂CH(CH₃)₂, *—O—(CH₂)₃CH₃, *—O—(CH₂)₂O—CH₃,

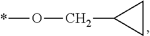 [SMILES: *OCC1CC1]

*—O—CH₂-Phenyl, *—N═S(O)(CH₃)₂, *—CH₃,

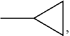

*—NH(CH₃), *—N(CH₃)₂, *—NH₂,

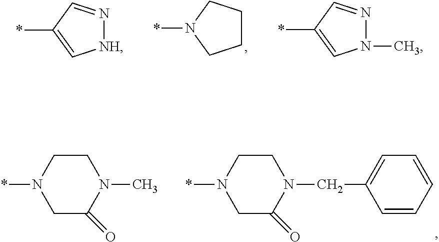

*—C(CH₃)₂—OH,

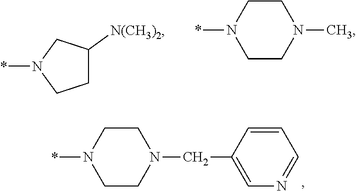 [SMILES: *N1CCC(N(C)C)C1 ; *N1CCN(C)CC1 ; *N1CCN(Cc2cccnc2)CC1]

*—NH—(CH₂)₂—NH—C(O)—CH₃, *—NH—(CH₂)₂-morpholino, *—NH—C(O)—CH₃, *—NH—C(O)—NH—CH₃, *—NH—C(O)—N(CH₃)₂, *—NO₂, *—NH—S(O)₂—CH₃, *—N═S(O)(CH₃)₂, or *—OH, *—O—(CH₂)₂—S(O)₂—CH₃, *-fluorine,

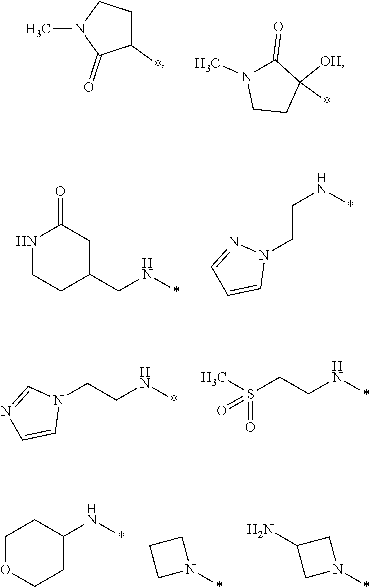

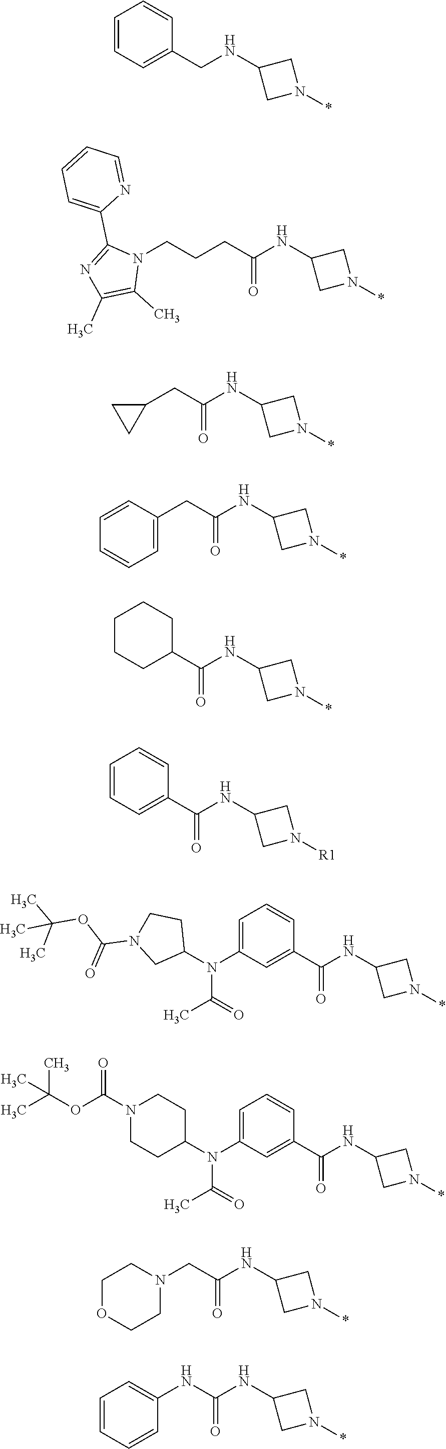

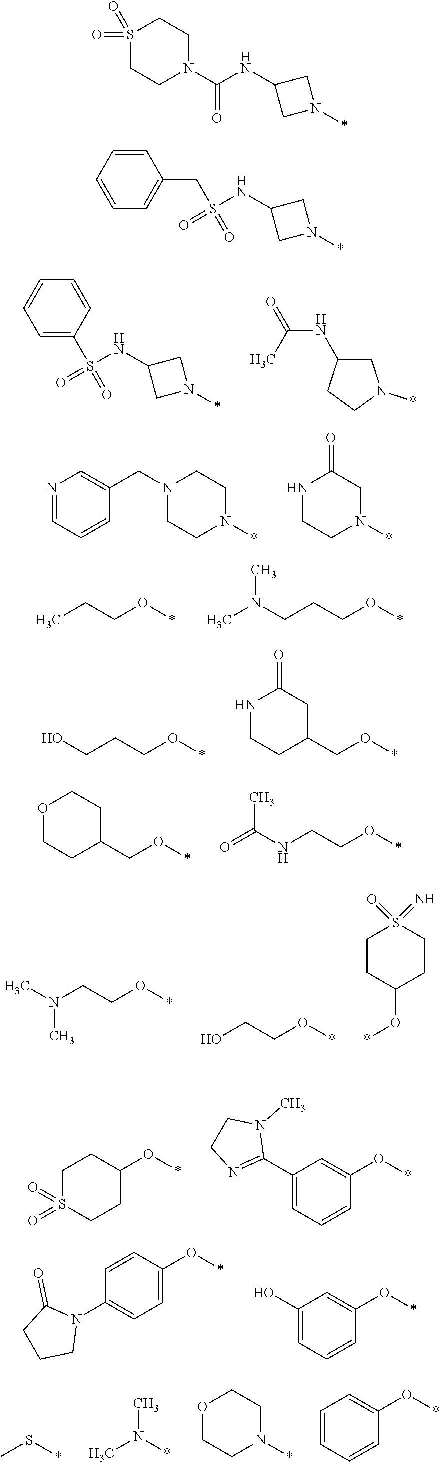

and
z is 1 or 2 and
x is 1 or 2 and wherein
A1 is selected from the group

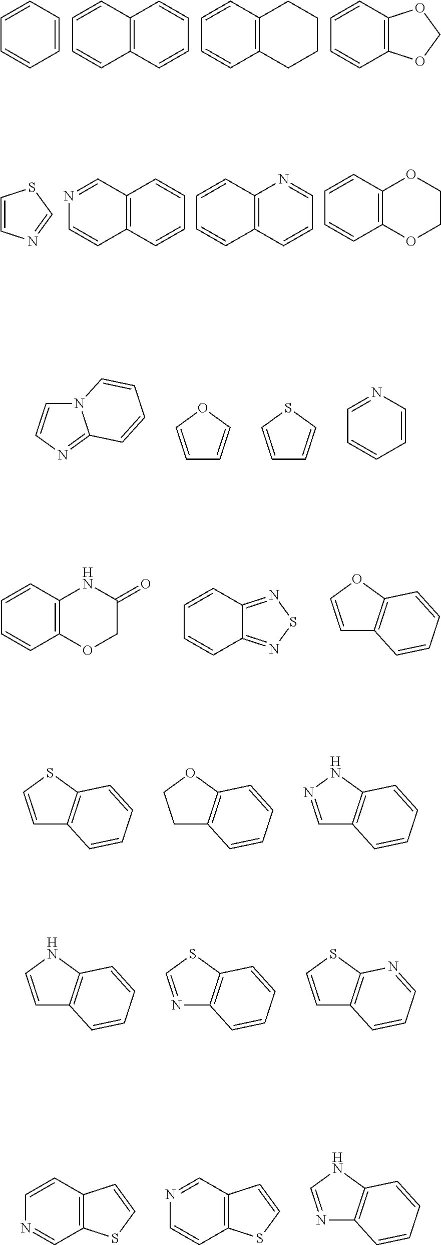

and
R² is selected from the group of hydrogen, hydroxy, oxo (═O), cyano, cyclopropyl, 1,1-dimethylcyclopropyl, —C(═CH₂)CH₃, —C(CH₃)═CHCH₃, —CH═CH—(CH₂)₂CH₃, CH═CHCH₃, —CH═CH— cyclopropyl), —C(O)NH₂, C(O)OCH₃, —S(O)₂CH₃, —OCH₃, —CH₂NH₂, a halogen atom (F, Cl; Br), and
w is 1 or 2 and
A2 is selected from the group

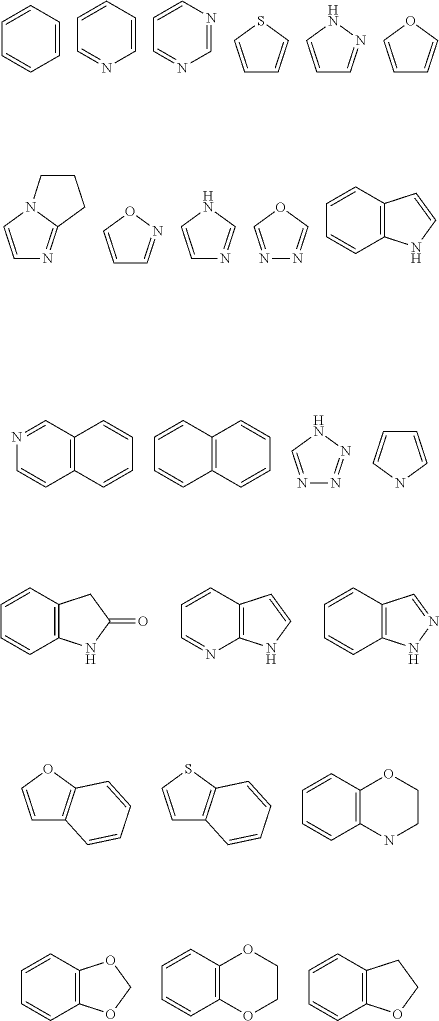

and
R³ is selected from the group of the following substituents
*—C(O)NH—(CH₂)₂CH₃
*—C(O)—N(CH₃)₂
*—C(O)—NH₂
*—C(O)—NH—(CH₂)₂N(CH₃)₂
*—CH₂—C(O)—NH₂ hydrogen
*—F, *—Cl, *—Br
*—C≡N; *—CF₃, *—CH₃, *—C₂H₅, *—CH═CH₂;
*—CH₂—CN; *—CH(CH₃)—NH₂; *—CH═CH—CN;
—C(O)—OH; *—C(O)—OCH₃; *—C(O)—CH₃; *—C(CH₃)₂—C(O)—OCH₃; *—C(CH₃)₂—CN; Oxo(=O); hydroxy;

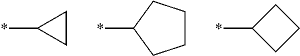

*—NH₂
*—NH—C(O)CH₃
*—NH—SO₂—CH₃
*—NH—C(O)—O—C(CH₃)₃

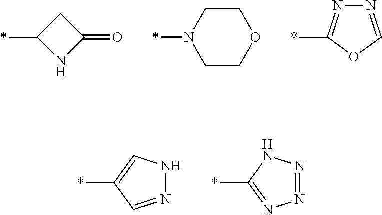

*—SO₂—CH₃
*—SO₂—N(CH₃)₂
*—SO₂—NH₂
*—O—CH₂—CH₃; *—O—(CH₂)₂—CH₃; *—O—CF₃;

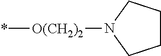

*—OCH₂-Cyclopropyl; *—OOH₃;
*—O(CH₂)₃—CH₃; *—OOH₂-Phenyl; *—O-Phenyl;
*—(OH₂)—OH
*—(OH₂)₂—OH
*—(OH₂)—O—CH₃
*—(OH₂)—O—CH₂—CH₃
*—CH(OH)—CH₂-Phenyl
*—CH(OH)—OH₂—CH₃
*—CH(OH)—(OH₂)₂—CH₃*—CH(OH)—(OH₂)₃—CH₃
*—CH(OH)—CH—(CH₃)₂
*—CH(OH)-Phenyl
*—CH(OH)—ON
*—CH(OH)—OH₂OH
*—CH(OH)—CF₃
*—CH(OH)—(CH₂)₂-Phenyl
*—CH(OH)—C≡CH
*—CH(NH₂)—CH₂—COOH
*—CH₂—NH—SO₂—CH₃
*—CH₂—NH—(CH₂)₃—CH₃
*—CH₂—NH—CH₃
*—CH₂—N(CH₃)₂
*—CH₂—NH—C₂H₅*—CH(CH₃)—NH₂
*—CH₂—NH₂
*—(CH₂)₂—NH₂
*—CH₂—NH—CH₂-Phenyl
*—CH₂—N(C₂H₅)₂
*—CH₂—NH-Cyclopropyl
*—CH₂—NH-Cyclobutyl
*—CH₂—NH-Cyclopentyl
*—CH₂—NH-Pyridyl
*—CH₂—NH-Phenyl
*—CH₂—NH—(CH₂)₂—OH
*—CH₂—N(CH₃)(CH₂)₂OH*—CH₂—NH—CH₂—CN
*—CH₂—N(CH₃)—CH₂—CN
*—CH₂—N(CH₃)—CH₂—CF₃
*—CH₂—N(CH₃)—CH₂—CF₂H
*—CH₂—NH—CH₂—CF₂H
*—CH₂—NH—CH₂—CF₃
*—CH₂—NH—(CH₂)₂—OCH₃

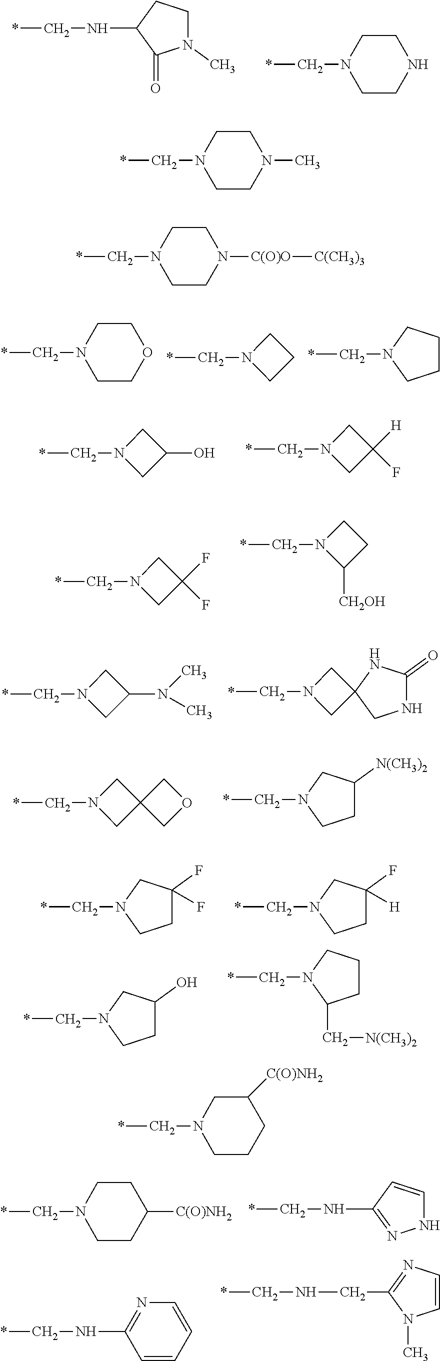

*—CH₂—NH—C(O)—O—C(CH₃)₃
*—(CH₂)₂—NH—C(O)—O—C(CH₃)₃
*—CH₂—NH—C(O)—CH₂—OH
*—CH₂—NH—C(O)—CH₂—OCH₃
*—CH—(CH₃)—NH—C(O)—O—C(CH₃)₃
*—CH₂—NH—C(O)—CH₃

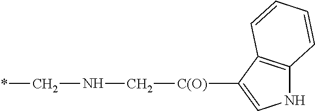

*—CH₂—NH—CH₂—C(O)—NH₂
*—CH₂—NH—CH₂—C(O)—N(CH₃)₂
*—CH₂—NH—CH₂—C(O)—OCH₃
*—CH₂—NH—CH₂—C(O)—NHCH₃
*—CH₂—NH—CH₂—C(O)—NH—(CH₂)₂—O—CH₃
*—CH₂—NH—CH₂—C(O)—NH—CH₂-Phenyl

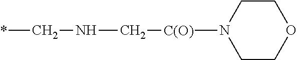

*—CH₂—NH—CH₂—C(O)—NH-Phenyl

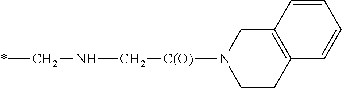

*—CH₂—NH—C(O)—CH₂—NH-Phenyl

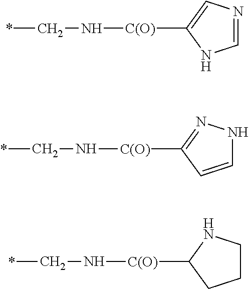

*—CH₂—NH—C(O)—CH₂—NH—CH₂—CF₃

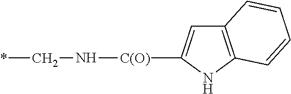

and
y is 1 or 2 and
k is 1 or 2 and
n is 0 or 1
and stereoisomers, tautomers, N-oxides, hydrates, solvates, and salts thereof, and mixtures of same.

In accordance with a further embodiment of the first aspect, the present invention covers the following compounds of general formula (I), supra, namely:
6,7-dimethoxy-2-methyl-N-[(1R)-1-(naphthalen-1-yl)ethyl]quinazolin-4-amine
N-[(1R)-1-(3-chlorophenyl)ethyl]-6,7-dimethoxy-2-methylquinazolin-4-amine
methyl 4-{1-[(6,7-dimethoxy-2-methylquinazolin-4-yl)amino]ethyl}-1-benzothiophene-2-carboxylate
N-[1-(1-benzofuran-7-yl)ethyl]-6,7-dimethoxy-2-methylquinazolin-4-amine
N-[1-(7-fluoro-1H-indazol-4-yl)ethyl]-6,7-dimethoxy-2-methylquinazolin-4-amine
N-[1-(6-fluoro-1H-indazol-4-yl)ethyl]-6,7-dimethoxy-2-methylquinazolin-4-amine
6,7-dimethoxy-2-methyl-N-[1-(5-methyl-1H-indazol-4-yl)ethyl]quinazolin-4-amine
6,7-dimethoxy-2-methyl-N-[1-(2-methyl-2H-indazol-7-yl)ethyl]quinazolin-4-amine
6,7-dimethoxy-2-methyl-N-[1-(2-methyl-2H-indazol-4-yl)ethyl]quinazolin-4-amine
6,7-dimethoxy-2-methyl-N-[1-(1-methyl-1H-indazol-7-yl)ethyl]quinazolin-4-amine
6,7-dimethoxy-2-methyl-N-[1-(5,6,7,8-tetrahydronaphthalen-1-yl)ethyl]quinazolin-4-amine
N-[(1R)-1-(4-fluorophenyl)ethyl]-6,7-dimethoxy-2-methylquinazolin-4-amine
6,7-dimethoxy-2-methyl-N-[1-(3-methyl-1H-indazol-4-yl)ethyl]quinazolin-4-amine
N-[1-(1,3-benzothiazol-4-yl)ethyl]-6,7-dimethoxy-2-methylquinazolin-4-amine
N-[1-(1-benzothiophen-7-yl)ethyl]-6,7-dimethoxy-2-methylquinazolin-4-amine
6,7-dimethoxy-2-methyl-N-[1-(6-methyl-1H-indazol-4-yl)ethyl]quinazolin-4-amine
6,7-dimethoxy-2-methyl-N-[1-(1-methyl-1H-indazol-4-yl)ethyl]quinazolin-4-amine
N-[1-(5-fluoro-1H-indazol-4-yl)ethyl]-6,7-dimethoxy-2-methylquinazolin-4-amine
N-[1-(2,3-dihydro-1,4-benzodioxin-6-yl)ethyl]-6,7-dimethoxy-2-methylquinazolin-4-amine
N-[1-(1-benzofuran-2-yl)ethyl]-6,7-dimethoxy-2-methylquinazolin-4-amine
N-[1-(2,3-dimethoxyphenyl)ethyl]-6,7-dimethoxy-2-methylquinazolin-4-amine
N-[1-(2,3-dihydro-1-benzofuran-4-yl)ethyl]-6,7-dimethoxy-2-methylquinazolin-4-amine
N-[1-(1,3-benzodioxol-5-yl)ethyl]-6,7-dimethoxy-2-methylquinazolin-4-amine
N-[1-(2,3-dihydro-1-benzofuran-5-yl)ethyl]-6,7-dimethoxy-2-methylquinazolin-4-amine
6,7-dimethoxy-2-methyl-N-[1-(5,6,7,8-tetrahydronaphthalen-2-yl)ethyl]quinazolin-4-amine
6,7-dimethoxy-2-methyl-N-[1-(2-methylimidazo[1,2-a]pyridin-3-yl)ethyl]quinazolin-4-amine
N-[1-(1-benzothiophen-3-yl)ethyl]-6,7-dimethoxy-2-methylquinazolin-4-amine
2-{1-[(6,7-dimethoxy-2-methylquinazolin-4-yl)amino]ethyl}-1-benzofuran-7-ol
6-bromo-2-methyl-N-[(1R)-1-phenylethyl]quinazolin-4-amine
6-{[dimethyl(oxido)-lambda6-sulfanylidene]amino}-2-methyl-N-[(1R)-1-phenylethyl]quinazolin-4-amine
6-bromo-N-[(1R)-1-(4-fluorophenyl)ethyl]-2-methylquinazolin-4-amine
6-{[dimethyl(oxido)-lambda6-sulfanylidene]amino}-N-[(1R)-1-(4-fluorophenyl)ethyl]-2-methylquinazolin-4-amine
6,7-dimethoxy-N-[1-(7-methoxy-1-benzofuran-2-yl)ethyl]-2-methylquinazolin-4-amine
6-{1-[(6,7-dimethoxy-2-methylquinazolin-4-yl)amino]ethyl}-2H-1,4-benzoxazin-3(4H)-one
6,7-dimethoxy-N-[1-(6-methoxy-2-naphthyl)ethyl]-2-methylquinazolin-4-amine
N-[(1R)-1-(5′-amino-2′-methylbiphenyl-3-yl)ethyl]-6,7-dimethoxy-2-methylquinazolin-4-amine
6,7-dimethoxy-2-methyl-N-{(1R)-1-[3-(pyrimidin-5-yl)phenyl]ethyl}quinazolin-4-amine
N-{(1R)-1-[3′-(cyclopropylmethoxy)biphenyl-3-yl]ethyl}-6,7-dimethoxy-2-methylquinazolin-4-amine
N-{(1R)-1-[3-(isoquinolin-5-yl)phenyl]ethyl}-6,7-dimethoxy-2-methylquinazolin-4-amine
N-[(1R)-1-(2′-chloro-6′-fluoro-3′-methylbiphenyl-3-yl)ethyl]-6,7-dimethoxy-2-methylquinazolin-4-amine
6,7-dimethoxy-2-methyl-N-{(1R)-1-[3-(5-methylpyridin-3-yl)phenyl]ethyl}quinazolin-4-amine
6,7-dimethoxy-2-methyl-N-{1-[5-(pyrimidin-5-yl)thiophen-2-yl]ethyl}quinazolin-4-amine
6,7-dimethoxy-2-methyl-N-[1-{5-[4-(morpholin-4-yl)phenyl]thiophen-2-yl}ethyl]quinazolin-4-amine
6,7-dimethoxy-2-methyl-N-[1-{5-[3-(morpholin-4-yl)phenyl]thiophen-2-yl}ethyl]quinazolin-4-amine
N-{1-[5-(isoquinolin-5-yl)thiophen-2-yl]ethyl}-6,7-dimethoxy-2-methylquinazolin-4-amine
6,7-dimethoxy-2-methyl-N-{1-[5-(5-methylpyridin-3-yl)thiophen-2-yl]ethyl}quinazolin-4-amine
6,7-dimethoxy-2-methyl-N-{1-[5-(2-propoxyphenyl)thiophen-2-yl]ethyl}quinazolin-4-amine
2-(5-{1-[(6,7-dimethoxy-2-methylquinazolin-4-yl)amino]ethyl}thiophen-2-yl)-N, N-dimethylbenzamide
6,7-dimethoxy-2-methyl-N-{1-[5-(1-methyl-1H-indol-5-yl)thiophen-2-yl]ethyl}quinazolin-4-amine
6,7-dimethoxy-N-[1-{5-[2-(methoxymethyl)phenyl]thiophen-2-yl}ethyl]-2-methylquinazolin-4-amine
3-(5-{1-[(6,7-dimethoxy-2-methylquinazolin-4-yl)amino]ethyl}thiophen-2-yl)-N, N-dimethylbenzamide
(5′-{1-[(6,7-dimethoxy-2-methylquinazolin-4-yl)amino]ethyl}-2,2′-bithiophen-5-yl)methanol
6,7-dimethoxy-2-methyl-N-{1-[5-(3-methylpyridin-4-yl)thiophen-2-yl]ethyl}quinazolin-4-amine
N-{1-[5-(1H-indol-6-yl)thiophen-2-yl]ethyl}-6,7-dimethoxy-2-methylquinazolin-4-amine
6,7-dimethoxy-2-methyl-N-{(1R)-1-[3′-(5-methyl-1,3,4-oxadiazol-2-yl)biphenyl-3-yl]ethyl}quinazolin-4-amine
6,7-dimethoxy-2-methyl-N-[(1R)-1-{3-[5-(methylsulfonyl)pyridin-3-yl]phenyl}ethyl]quinazolin-4-amine
5-(3-{(1R)-1-[(6,7-dimethoxy-2-methylquinazolin-4-yl)amino]ethyl}phenyl)-1,3-dihydro-2H-indol-2-one
N-{(1R)-1-[3-(2,2-dimethylcyclopropyl)phenyl]ethyl}-6,7-dimethoxy-2-methylquinazolin-4-amine
6,7-dimethoxy-2-methyl-N-{(1R)-1-[4′-(5-methyl-1,3,4-oxadiazol-2-yl)biphenyl-3-yl]ethyl}quinazolin-4-amine
6,7-dimethoxy-2-methyl-N-{(1R)-1-[3-(1H-pyrrolo[2,3-b]pyridin-5-yl)phenyl]ethyl}quinazolin-4-amine
3′-{(1R)-1-[(6,7-dimethoxy-2-methylquinazolin-4-yl)amino]ethyl}biphenyl-3-sulfonamide
N-{(1R)-1-[3-(2-aminopyrimidin-5-yl)phenyl]ethyl}-6,7-dimethoxy-2-methylquinazolin-4-amine
N-[(1R)-1-{3-[(E)-2-cyclopropylethenyl]phenyl}ethyl]-6,7-dimethoxy-2-methylquinazolin-4-amine
N-{(1R)-1-[2′-(ethoxymethyl)biphenyl-3-yl]ethyl}-6,7-dimethoxy-2-methylquinazolin-4-amine
N-[(1R)-1-(3′-fluoro-5′-methoxybiphenyl-3-yl)ethyl]-6,7-dimethoxy-2-methylquinazolin-4-amine
6,7-dimethoxy-N-{(1R)-1-[3-(5-methoxy-1-benzofuran-2-yl)phenyl]ethyl}-2-methylquinazolin-4-amine
N-[(1R)-1-(2′-butoxy-6′-fluorobiphenyl-3-yl)ethyl]-6,7-dimethoxy-2-methylquinazolin-4-amine
5-(3-{(1R)-1-[(6,7-dimethoxy-2-methylquinazolin-4-yl)amino]ethyl}phenyl)pyridin-2-ol
2-(3′-{(1R)-1-[(6,7-dimethoxy-2-methylquinazolin-4-yl)amino]ethyl}biphenyl-4-yl)-2-methylpropanenitrile
6,7-dimethoxy-2-methyl-N-[1-(5-phenylthiophen-2-yl)ethyl]quinazolin-4-amine
N-[(3′-{(1R)-1-[(6,7-dimethoxy-2-methylquinazolin-4-yl)amino]ethyl}biphenyl-3-yl)methyl]methanesulfonamide
N-[(3′-{(1R)-1-[(6,7-dimethoxy-2-methylquinazolin-4-yl)amino]ethyl}biphenyl-4-yl)methyl]methanesulfonamide
3′-{(1R)-1-[(6,7-dimethoxy-2-methylquinazolin-4-yl)amino]ethyl}-N-propylbiphenyl-4-carboxamide
3′-{(1R)-1-[(6,7-dimethoxy-2-methylquinazolin-4-yl)amino]ethyl}-N-[2-(dimethylamino)ethyl]biphenyl-4-carboxamide
6,7-dimethoxy-2-methyl-N-{(1R)-1-[3-(1H-pyrazol-3-yl)phenyl]ethyl}quinazolin-4-amine
N-[(1R)-1-{3-[(2E)-but-2-en-2-yl]phenyl}ethyl]-6,7-dimethoxy-2-methylquinazolin-4-amine
N-[(1R)-1-(5′-chloro-2′-propoxybiphenyl-3-yl)ethyl]-6,7-dimethoxy-2-methylquinazolin-4-amine
6,7-dimethoxy-2-methyl-N-[(1R)-1-{3-[(1E)-3-phenylprop-1-en-1-yl]phenyl}ethyl]quinazolin-4-amine
6,7-dimethoxy-2-methyl-N-{(1R)-1-[4′-(morpholin-4-yl)biphenyl-3-yl]ethyl}quinazolin-4-amine
6,7-dimethoxy-2-methyl-N-{(1R)-1-[3′-(morpholin-4-yl)biphenyl-3-yl]ethyl}quinazolin-4-amine
N-{(1R)-1-[2′-(benzyloxy)biphenyl-3-yl]ethyl}-6,7-dimethoxy-2-methylquinazolin-4-amine
6,7-dimethoxy-2-methyl-N-{(1R)-1-[4′-(methylsulfonyl)biphenyl-3-yl]ethyl}quinazolin-4-amine
6,7-dimethoxy-2-methyl-N-{(1R)-1-[2′-(trifluoromethoxy)biphenyl-3-yl]ethyl}quinazolin-4-amine
6,7-dimethoxy-2-methyl-N-{(1R)-1-[3′-(trifluoromethoxy)biphenyl-3-yl]ethyl}quinazolin-4-amine
N-{(1R)-1-[3-(1H-indol-5-yl)phenyl]ethyl}-6,7-dimethoxy-2-methylquinazolin-4-amine
N-{(1R)-1-[3-(furan-3-yl)phenyl]ethyl}-6,7-dimethoxy-2-methylquinazolin-4-amine
N-{(1R)-1-[3-(1-benzothiophen-3-yl)phenyl]ethyl}-6,7-dimethoxy-2-methylquinazolin-4-amine
6,7-dimethoxy-2-methyl-N-{(1R)-1-[3-(1-methyl-1H-indol-2-yl)phenyl]ethyl}quinazolin-4-amine
6,7-dimethoxy-2-methyl-N-[(1R)-1-{3-[(1E)-pent-1-en-1-yl]phenyl}ethyl]quinazolin-4-amine
N-[(1R)-1-{3-[(E)-2-cyclohexylethenyl]phenyl}ethyl]-6,7-dimethoxy-2-methylquinazolin-4-amine
6,7-dimethoxy-2-methyl-N-[(1R)-1-(2′-phenoxybiphenyl-3-yl)ethyl]quinazolin-4-amine
tert-butyl (3′-{(1R)-1-[(6,7-dimethoxy-2-methylquinazolin-4-yl)amino]ethyl}biphenyl-4-yl)carbamate
(2E)-3-(3′-{(1R)-1-[(6,7-dimethoxy-2-methylquinazolin-4-yl)amino]ethyl}biphenyl-3-yl)prop-2-enenitrile
N-[(1R)-1-(2′,4′-dimethylbiphenyl-3-yl)ethyl]-6,7-dimethoxy-2-methylquinazolin-4-amine
1-[5-(3-{(1R)-1-[(6,7-dimethoxy-2-methylquinazolin-4-yl)amino]ethyl}phenyl)thiophen-2-yl]ethanone
N-{(1R)-1-[3-(1,3-benzodioxol-5-yl)phenyl]ethyl}-6,7-dimethoxy-2-methylquinazolin-4-amine
N-{(1R)-1-[4′-(benzyloxy)biphenyl-3-yl]ethyl}-6,7-dimethoxy-2-methylquinazolin-4-amine
N-{1-[5-(2,3-dihydro-1,4-benzodioxin-6-yl)thiophen-2-yl]ethyl}-6,7-dimethoxy-2-methylquinazolin-4-amine
6,7-dimethoxy-N-[(1R)-1-(3′-methoxybiphenyl-3-yl)ethyl]-2-methylquinazolin-4-amine
6,7-dimethoxy-2-methyl-N-{(1R)-1-[3′-(trifluoromethyl)biphenyl-3-yl]ethyl}quinazolin-4-amine
3′-{(1R)-1-[(6,7-dimethoxy-2-methylquinazolin-4-yl)amino]ethyl}-N, N-di methylbiphenyl-2-sulfonamide
6,7-dimethoxy-2-methyl-N-[(1R)-1-(2′-propoxybiphenyl-3-yl)ethyl]quinazolin-4-amine
3′-{(1R)-1-[(6,7-dimethoxy-2-methylquinazolin-4-yl)amino]ethyl}-N, N-dimethylbiphenyl-2-carboxamide
6,7-dimethoxy-N-{(1R)-1-[2′-(methoxymethyl)biphenyl-3-yl]ethyl}-2-methylquinazolin-4-amine
6,7-dimethoxy-2-methyl-N-{(1R)-1-[3-(1-methyl-1H-indol-5-yl)phenyl]ethyl}quinazolin-4-amine
3′-{(1R)-1-[(6,7-dimethoxy-2-methylquinazolin-4-yl)amino]ethyl}-N, N-dimethylbiphenyl-3-carboxamide
[5-(3-{(1R)-1-[(6,7-dimethoxy-2-methylquinazolin-4-yl)amino]ethyl}phenyl)thiophen-2-yl]methanol
6,7-dimethoxy-2-methyl-N-{(1R)-1-[3-(3-methylpyridin-4-yl)phenyl]ethyl}quinazolin-4-amine
N-{(1R)-1-[3-(1H-indol-6-yl)phenyl]ethyl}-6,7-dimethoxy-2-methylquinazolin-4-amine
N-{(1R)-1-[3-(1H-indol-4-yl)phenyl]ethyl}-6,7-dimethoxy-2-methylquinazolin-4-amine
6,7-dimethoxy-N-{(1R)-1-[3-(2-methoxypyrimidin-5-yl)phenyl]ethyl}-2-methylquinazolin-4-amine
6,7-dimethoxy-2-methyl-N-{(1R)-1-[3′-(methylsulfonyl)biphenyl-3-yl]ethyl}quinazolin-4-amine
N-{(1R)-1-[3-(2,3-dihydro-1-benzofuran-5-yl)phenyl]ethyl}-6,7-dimethoxy-2-methylquinazolin-4-amine
6,7-dimethoxy-2-methyl-N-[(1R)-1-{3-[(E)-2-phenylethenyl]phenyl}ethyl]quinazolin-4-amine
3′-{(1R)-1-[(6,7-dimethoxy-2-methylquinazolin-4-yl)amino]ethyl}-N, N-dimethylbiphenyl-4-carboxamide
6,7-dimethoxy-2-methyl-N-[(1R)-1-{3-[(1E)-prop-1-en-1-yl]phenyl}ethyl]quinazolin-4-amine
N-{(1R)-1-[3-(cyclopent-1-en-1-yl)phenyl]ethyl}-6,7-dimethoxy-2-methylquinazolin-4-amine
N-(3′-{(1R)-1-[(6,7-dimethoxy-2-methylquinazolin-4-yl)amino]ethyl}biphenyl-3-yl)methanesulfonamide
N-(3′-{(1R)-1-[(6,7-dimethoxy-2-methylquinazolin-4-yl)amino]ethyl}biphenyl-2-yl)acetamide
6,7-dimethoxy-2-methyl-N-{(1R)-1-[2′-(methylsulfonyl)biphenyl-3-yl]ethyl}quinazolin-4-amine
N-(3′-{(1R)-1-[(6,7-dimethoxy-2-methylquinazolin-4-yl)amino]ethyl}biphenyl-2-yl)methanesulfonamide
N-{1-[5-(3,5-dichlorophenyl)thiophen-2-yl]ethyl}-6,7-dimethoxy-2-methylquinazolin-4-amine
N-{(1R)-1-[3′-(benzyloxy)biphenyl-3-yl]ethyl}-6,7-dimethoxy-2-methylquinazolin-4-amine
N-[(1R)-1-(3′,5′-dichlorobiphenyl-3-yl)ethyl]-6,7-dimethoxy-2-methylquinazolin-4-amine
N-{(1R)-1-[3-(2,3-dihydro-1,4-benzodioxin-6-yl)phenyl]ethyl}-6,7-dimethoxy-2-methylquinazolin-4-amine
6,7-dimethoxy-2-methyl-N-[1-{5-[3-(5-methyl-1,3,4-oxadiazol-2-yl)phenyl]thiophen-2-yl}ethyl]quinazolin-4-amine
6,7-dimethoxy-2-methyl-N-[1-{5-[5-(methylsulfonyl)pyridin-3-yl]thiophen-2-yl}ethyl]quinazolin-4-amine
6,7-dimethoxy-2-methyl-N-[1-{5-[4-(5-methyl-1,3,4-oxadiazol-2-yl)phenyl]thiophen-2-yl}ethyl]quinazolin-4-amine
6,7-dimethoxy-2-methyl-N-{1-[5-(1H-pyrrolo[2,3-b]pyridin-5-yl)thiophen-2-yl]ethyl}quinazolin-4-amine
3-(5-{1-[(6,7-dimethoxy-2-methylquinazolin-4-yl)amino]ethyl}thiophen-2-yl)benzenesulfonamide
N-{1-[5-(2-aminopyrimidin-5-yl)thiophen-2-yl]ethyl}-6,7-dimethoxy-2-methylquinazolin-4-amine
N-[1-{5-[(E)-2-cyclopropylethenyl]thiophen-2-yl}ethyl]-6,7-dimethoxy-2-methylquinazolin-4-amine
N-[1-{5-[2-(ethoxymethyl)phenyl]thiophen-2-yl}ethyl]-6,7-dimethoxy-2-methylquinazolin-4-amine
N-{1-[5-(3-fluoro-5-methoxyphenyl)thiophen-2-yl]ethyl}-6,7-dimethoxy-2-methylquinazolin-4-amine
N-[1-{5-[3-(benzyloxy)phenyl]thiophen-2-yl}ethyl]-6,7-dimethoxy-2-methylquinazolin-4-amine
N-{1-[5-(2-butoxy-6-fluorophenyl)thiophen-2-yl]ethyl}-6,7-dimethoxy-2-methylquinazolin-4-amine
2-[4-(5-{1-[(6,7-dimethoxy-2-methylquinazolin-4-yl)amino]ethyl}thiophen-2-yl)phenyl]-2-methylpropanenitrile
N-[4-(5-{1-[(6,7-dimethoxy-2-methylquinazolin-4-yl)amino]ethyl}thiophen-2-yl)benzyl]acetamide
N-[3-(5-{1-[(6,7-dimethoxy-2-methylquinazolin-4-yl)amino]ethyl}thiophen-2-yl)benzyl]methanesulfonamide
N-[4-(5-{1-[(6,7-dimethoxy-2-methylquinazolin-4-yl)amino]ethyl}thiophen-2-yl)benzyl]methanesulfonamide
4-(5-{1-[(6,7-dimethoxy-2-methylquinazolin-4-yl)amino]ethyl}thiophen-2-yl)-N-propylbenzamide
4-(5-{1-[(6,7-dimethoxy-2-methylquinazolin-4-yl)amino]ethyl}thiophen-2-yl)-N-[2-(dimethylamino)ethyl]benzamide
N-[1-{5-[(2E)-but-2-en-2-yl]thiophen-2-yl}ethyl]-6,7-dimethoxy-2-methylquinazolin-4-amine
N-{1-[5-(5-chloro-2-propoxyphenyl)thiophen-2-yl]ethyl}-6,7-dimethoxy-2-methylquinazolin-4-amine
6,7-dimethoxy-2-methyl-N-[1-{5-[(1E)-3-phenylprop-1-en-1-yl]thiophen-2-yl}ethyl]quinazolin-4-amine
N-{1-[5-(5-amino-2-methylphenyl)thiophen-2-yl]ethyl}-6,7-dimethoxy-2-methylquinazolin-4-amine
N-{1-[5-(3,5-di methyl-1,2-oxazol-4-yl)thiophen-2-yl]ethyl}-6,7-dimethoxy-2-methylquinazolin-4-amine
6,7-dimethoxy-2-methyl-N-[1-{5-[2-(methylsulfonyl)phenyl]thiophen-2-yl}ethyl]quinazolin-4-amine
6,7-dimethoxy-2-methyl-N-[1-{5-[4-(methylsulfonyl)phenyl]thiophen-2-yl}ethyl]quinazolin-4-amine
6,7-dimethoxy-2-methyl-N-[1-{5-[2-(trifluoromethoxy)phenyl]thiophen-2-yl}ethyl]quinazolin-4-amine
6,7-dimethoxy-2-methyl-N-[1-{5-[3-(trifluoromethoxy)phenyl]thiophen-2-yl}ethyl]quinazolin-4-amine
N-{1-[5-(1H-indol-5-yl)thiophen-2-yl]ethyl}-6,7-dimethoxy-2-methylquinazolin-4-amine
N-{1-[5-(furan-3-yl)thiophen-2-yl]ethyl}-6,7-dimethoxy-2-methylquinazolin-4-amine
N-{1-[5-(1-benzothiophen-3-yl)thiophen-2-yl]ethyl}-6,7-dimethoxy-2-methylquinazolin-4-amine
6,7-dimethoxy-2-methyl-N-{1-[5-(1-methyl-1H-indol-2-yl)thiophen-2-yl]ethyl}quinazolin-4-amine
6,7-dimethoxy-2-methyl-N-[1-{5-[(1E)-pent-1-en-1-yl]thiophen-2-yl}ethyl]quinazolin-4-amine
N-[1-{5-[(E)-2-cyclohexylethenyl]thiophen-2-yl}ethyl]-6,7-dimethoxy-2-methylquinazolin-4-amine
6,7-dimethoxy-2-methyl-N-{1-[5-(2-phenoxyphenyl)thiophen-2-yl]ethyl}quinazolin-4-amine
tert-butyl [4-(5-{1-[(6,7-dimethoxy-2-methylquinazolin-4-yl)amino]ethyl}thiophen-2-yl)phenyl]carbamate
(2E)-3-[3-(5-{1-[(6,7-dimethoxy-2-methylquinazolin-4-yl)amino]ethyl}thiophen-2-yl)phenyl]prop-2-enenitrile
N-{1-[5-(2,4-dimethylphenyl)thiophen-2-yl]ethyl}-6,7-dimethoxy-2-methylquinazolin-4-amine
1-(5′-{1-[(6,7-dimethoxy-2-methylquinazolin-4-yl)amino]ethyl}-2,2′-bithiophen-5-yl)ethanone
N-{1-[5-(1,3-benzodioxol-5-yl)thiophen-2-yl]ethyl}-6,7-dimethoxy-2-methylquinazolin-4-amine
6,7-dimethoxy-2-methyl-N-{1-[5-(4-methyl-3,4-dihydro-2H-1,4-benzoxazin-7-yl)thiophen-2-yl]ethyl}quinazolin-4-amine
N-[3-(5-{1-[(6,7-dimethoxy-2-methylquinazolin-4-yl)amino]ethyl}thiophen-2-yl)phenyl]methanesulfonamide
N-[2-(5-{1-[(6,7-dimethoxy-2-methylquinazolin-4-yl)amino]ethyl}thiophen-2-yl)phenyl]acetamide
6,7-dimethoxy-N-{1-[5-(3-methoxyphenyl)thiophen-2-yl]ethyl}-2-methylquinazolin-4-amine
6,7-dimethoxy-2-methyl-N-[1-{5-[3-(trifluoromethyl)phenyl]thiophen-2-yl}ethyl]quinazolin-4-amine
2-(5-{1-[(6,7-dimethoxy-2-methylquinazolin-4-yl)amino]ethyl}thiophen-2-yl)-N, N-dimethylbenzenesulfonamide
N-[1-{5-[3-(cyclopropylmethoxy)phenyl]thiophen-2-yl}ethyl]-6,7-dimethoxy-2-methylquinazolin-4-amine
N-{1-[5-(1H-indol-4-yl)thiophen-2-yl]ethyl}-6,7-dimethoxy-2-methylquinazolin-4-amine
6,7-dimethoxy-N-{1-[5-(2-methoxypyrimidin-5-yl)thiophen-2-yl]ethyl}-2-methylquinazolin-4-amine
6,7-dimethoxy-2-methyl-N-[1-{5-[3-(methylsulfonyl)phenyl]thiophen-2-yl}ethyl]quinazolin-4-amine
N-{1-[5-(2,3-dihydro-1-benzofuran-5-yl)thiophen-2-yl]ethyl}-6,7-dimethoxy-2-methylquinazolin-4-amine
6,7-dimethoxy-2-methyl-N-[1-{5-[(E)-2-phenylethenyl]thiophen-2-yl}ethyl]quinazolin-4-amine
4-(5-{1-[(6,7-dimethoxy-2-methylquinazolin-4-yl)amino]ethyl}thiophen-2-yl)-N, N-dimethylbenzamide
6,7-dimethoxy-2-methyl-N-[1-{5-[(1E)-prop-1-en-1-yl]thiophen-2-yl}ethyl]quinazolin-4-amine methyl 2-(5-{1-[(6,7-dimethoxy-2-methylquinazolin-4-yl)amino]ethyl}thiophen-2-yl)benzoate
1-[2-(5-{1-[(6,7-dimethoxy-2-methylquinazolin-4-yl)amino]ethyl}thiophen-2-yl)benzyl]piperidine-4-carboxamide
2-[2-(5-{1-[(6,7-dimethoxy-2-methylquinazolin-4-yl)amino]ethyl}thiophen-2-yl)phenyl]ethanol
2-[3-(5-{1-[(6,7-dimethoxy-2-methylquinazolin-4-yl)amino]ethyl}thiophen-2-yl)phenyl]ethanol
6,7-dimethoxy-2-methyl-N-[1-{5-[2-(2-oxa-6-azaspiro[3.3]hept-6-ylmethyl)phenyl]thiophen-2-yl}ethyl]quinazolin-4-amine
N-[1-(5-bromo-4-methylthiophen-2-yl)ethyl]-6,7-dimethoxy-2-methylquinazolin-4-amine
6,7-dimethoxy-2-methyl-N-[1-{5-[2-(pyrrolidin-1-ylmethyl)phenyl]thiophen-2-yl}ethyl]quinazolin-4-amine
N-[1-{5-[2-({2-[(dimethylamino)methyl]pyrrolidin-1-yl}methyl)phenyl]thiophen-2-yl}ethyl]-6,7-dimethoxy-2-methylquinazolin-4-amine
2-methyl-N-[(1R)-1-(naphthalen-1-yl)ethyl]quinazolin-4-amine
N-[(1R)-1-(4-bromophenyl)ethyl]-6,7-dimethoxy-2-methylquinazolin-4-amine
6,7-dimethoxy-2-methyl-N-{(1R)-1-[4-(methylsulfonyl)phenyl]ethyl}quinazolin-4-amine
4-{(1R)-1-[(6,7-dimethoxy-2-methylquinazolin-4-yl)amino]ethyl}benzonitrile
6,7-dimethoxy-2-methyl-N-[(1R)-1-(3-methylphenyl)ethyl]quinazolin-4-amine
N-[(1R)-1-(3-bromophenyl)ethyl]-6,7-dimethoxy-2-methylquinazolin-4-amine
4-{(1R)-1-[(6,7-dimethoxy-2-methylquinazolin-4-yl)amino]ethyl}benzamide
N-[(1R)-1-(biphenyl-3-yl)ethyl]-6,7-dimethoxy-2-methylquinazolin-4-amine
3-{(1R)-1-[(6,7-dimethoxy-2-methylquinazolin-4-yl)amino]ethyl}benzonitrile
6,7-dimethoxy-2-methyl-N-[(1R)-1-(4-methylphenyl)ethyl]quinazolin-4-amine
N-[(1R)-1-(biphenyl-4-yl)ethyl]-6,7-dimethoxy-2-methylquinazolin-4-amine
N-[(1R)-1-(4-cyclopropylphenyl)ethyl]-6,7-dimethoxy-2-methylquinazolin-4-amine
6,7-dimethoxy-2-methyl-N-{(1R)-1-[3-(methylsulfonyl)phenyl]ethyl}quinazolin-4-amine
3-{(1R)-1-[(6,7-dimethoxy-2-methylquinazolin-4-yl)amino]ethyl}benzamide
6,7-dimethoxy-2-methyl-N-{(1R)-1-[3-(1H-pyrazol-4-yl)phenyl]ethyl}quinazolin-4-amine
6,7-dimethoxy-2-methyl-N-{(1R)-1-[4-(1-methyl-1,2,3,6-tetrahydropyridin-4-yl)phenyl]ethyl}quinazolin-4-amine
6,7-dimethoxy-2-methyl-N-{(1R)-1-[3-(1-methyl-1,2,3,6-tetrahydropyridin-4-yl)phenyl]ethyl}quinazolin-4-amine
6,7-dimethoxy-2-methyl-N-{(1R)-1-[4-(prop-1-en-2-yl)phenyl]ethyl}quinazolin-4-amine
N-[(1R)-1-(3-cyclopropylphenyl)ethyl]-6,7-dimethoxy-2-methylquinazolin-4-amine
N-[1-(1-benzothiophen-4-yl)ethyl]-6,7-dimethoxy-2-methylquinazolin-4-amine
6,7-dimethoxy-2-methyl-N-[(1R)-1-phenylethyl]quinazolin-4-amine
6,7-dimethoxy-2-methyl-N-[1-(thiophen-2-yl)ethyl]quinazolin-4-amine
N-[1-(5-bromofuran-2-yl)ethyl]-6,7-dimethoxy-2-methylquinazolin-4-amine
N-[1-(5-bromothiophen-2-yl)ethyl]-6,7-dimethoxy-2-methylquinazolin-4-amine
1-[2-(5-{1-[(6,7-dimethoxy-2-methylquinazolin-4-yl)amino]ethyl}thiophen-2-yl)benzyl]pyrrolidin-3-ol
N-{1-[5-(2-{[(3S)-3-fluoropyrrolidin-1-yl]methyl}phenyl)thiophen-2-yl]ethyl}-6,7-dimethoxy-2-methylquinazolin-4-amine
6,7-dimethoxy-2-methyl-N-[1-(quinolin-5-yl)ethyl]quinazolin-4-amine
6,7-dimethoxy-2-methyl-N-[1-(5-phenylfuran-2-yl)ethyl]quinazolin-4-amine
N-[1-(5-bromo-2,3-dihydro-1-benzofuran-7-yl)ethyl]-6,7-dimethoxy-2-methylquinazolin-4-amine
6,7-dimethoxy-2-methyl-N-[1-(3-phenoxyphenyl)ethyl]quinazolin-4-amine
6,7-dimethoxy-2-methyl-N-{1-[3-(2H-tetrazol-5-yl)phenyl]ethyl}quinazolin-4-amine
6,7-dimethoxy-2-methyl-N-[1-(quinolin-8-yl)ethyl]quinazolin-4-amine
2-[4-(5-{1-[(6,7-dimethoxy-2-methylquinazolin-4-yl)amino]ethyl}thiophen-2-yl)-1H-pyrazol-1-yl]ethanol
N-{1-[5-(6,7-dihydro-5H-pyrrolo[1,2-a]imidazol-3-yl)thiophen-2-yl]ethyl}-6,7-dimethoxy-2-methylquinazolin-4-amine
6,7-dimethoxy-2-methyl-N-[1-(5-{1-[2-(pyrrolidin-1-yl)ethyl]-1H-pyrazol-4-yl}thiophen-2-yl)ethyl]quinazolin-4-amine
N-{1-[5-(1-cyclopentyl-1H-pyrazol-4-yl)thiophen-2-yl]ethyl}-6,7-dimethoxy-2-methylquinazolin-4-amine
6,7-dimethoxy-2-methyl-N-{1-[5-(1H-pyrazol-3-yl)thiophen-2-yl]ethyl}quinazolin-4-amine
N-[1-(5-{2-[(3,3-difluoropyrrolidin-1-yl)methyl]phenyl}thiophen-2-yl)ethyl]-6,7-dimethoxy-2-methylquinazolin-4-amine
6,7-dimethoxy-2-methyl-N-[1-(5-phenylfuran-2-yl)ethyl]quinazolin-4-amine
N-[1-(5-bromo-2,3-dihydro-1-benzofuran-7-yl)ethyl]-6,7-dimethoxy-2-methylquinazolin-4-amine
2-[1-(5-{1-[(6,7-dimethoxy-2-methylquinazolin-4-yl)amino]ethyl}furan-2-yl)-1H-pyrazol-3-yl]ethanol
5-{1-[(6,7-dimethoxy-2-methylquinazolin-4-yl)amino]ethyl}pyridin-2(1H)-one
6,7-dimethoxy-2-methyl-N-[1-(3-phenoxyphenyl)ethyl]quinazolin-4-amine
N-[1-(2,1,3-benzothiadiazol-5-yl)ethyl]-6,7-dimethoxy-2-methylquinazolin-4-amine
6,7-dimethoxy-2-methyl-N-[(1R)-1-(quinolin-8-yl)ethyl]quinazolin-4-amine
N-{1-[5-(cyclopent-1-en-1-yl)thiophen-2-yl]ethyl}-6,7-dimethoxy-2-methylquinazolin-4-amine
N-{1-[5-(2-ethoxyphenyl)thiophen-2-yl]ethyl}-6,7-dimethoxy-2-methylquinazolin-4-amine
N-{1-[5-(4-fluoronaphthalen-1-yl)thiophen-2-yl]ethyl}-6,7-dimethoxy-2-methylquinazolin-4-amine
N-[1-{5-[2-(aminomethyl)-4-fluorophenyl]thiophen-2-yl}ethyl]-6,7-dimethoxy-2-methylquinazolin-4-amine
N-{1-[5-(3,6-dihydro-2H-pyran-4-yl)thiophen-2-yl]ethyl}-6,7-dimethoxy-2-methylquinazolin-4-amine
tert-butyl {[5-(5-{1-[(6,7-dimethoxy-2-methylquinazolin-4-yl)amino]ethyl}thiophen-2-yl)furan-2-yl]methyl}carbamate
methyl 3-(5-{1-[(6,7-dimethoxy-2-methylquinazolin-4-yl)amino]ethyl}thiophen-2-yl)-1-methyl-1H-pyrazole-5-carboxylate
N-{1-[5-(2-{[3-(dimethylamino)pyrrolidin-1-yl]methyl}phenyl)thiophen-2-yl]ethyl}-6,7-dimethoxy-2-methylquinazolin-4-amine
N-[1-(5-bromothiophen-3-yl)ethyl]-6,7-dimethoxy-2-methylquinazolin-4-amine
2-(5-{1-[(6,7-dimethoxy-2-methylquinazolin-4-yl)amino]ethyl}thiophen-2-yl)benzamide
3-(5-{1-[(6,7-dimethoxy-2-methylquinazolin-4-yl)amino]ethyl}thiophen-2-yl)benzamide
4-(5-{1-[(6,7-dimethoxy-2-methylquinazolin-4-yl)amino]ethyl}thiophen-2-yl)benzamide
N-{1-[5-(2-aminophenyl)thiophen-2-yl]ethyl}-6,7-dimethoxy-2-methylquinazolin-4-amine
[2-(5-{1-[(6,7-dimethoxy-2-methylquinazolin-4-yl)amino]ethyl}thiophen-2-yl)phenyl]methanol
2-(5-{1-[(6,7-dimethoxy-2-methylquinazolin-4-yl)amino]ethyl}thiophen-2-yl)benzonitrile
N-{1-[5-(1H-indazol-7-yl)thiophen-2-yl]ethyl}-6,7-dimethoxy-2-methylquinazolin-4-amine
N-{1-[5-(1H-indazol-4-yl)thiophen-2-yl]ethyl}-6,7-dimethoxy-2-methylquinazolin-4-amine
N-{1-[5-(2-ethenylphenyl)thiophen-2-yl]ethyl}-6,7-dimethoxy-2-methylquinazolin-4-amine
2-[4-(5-{1-[(6,7-dimethoxy-2-methylquinazolin-4-yl)amino]ethyl}thiophen-2-yl)-1H-pyrazol-1-yl]acetamide
6,7-dimethoxy-2-methyl-N-[1-(5-{2-[(methylamino)methyl]phenyl}thiophen-2-yl)ethyl]quinazolin-4-amine
2-[4-(4-{1-[(6,7-dimethoxy-2-methylquinazolin-4-yl)amino]ethyl}thiophen-2-yl)-1H-pyrazol-1-yl]ethanol
N-[1-{5-[2-(aminomethyl)phenyl]thiophen-2-yl}ethyl]-6,7-dimethoxy-2-methylquinazolin-4-amine
N-{1-[5-(6,7-dihydro-5H-pyrrolo[1,2-a]imidazol-3-yl)thiophen-3-yl]ethyl}-6,7-dimethoxy-2-methylquinazolin-4-amine
6,7-dimethoxy-2-methyl-N-[1-(5-{1-[2-(pyrrolidin-1-yl)ethyl]-1H-pyrazol-4-yl}thiophen-3-yl)ethyl]quinazolin-4-amine
N-[1-{5-[2-(aminomethyl)-4-fluorophenyl]thiophen-3-yl}ethyl]-6,7-dimethoxy-2-methylquinazolin-4-amine
2-[4-(3-{(1R)-1-[(6,7-dimethoxy-2-methylquinazolin-4-yl)amino]ethyl}phenyl)-1H-pyrazol-1-yl]ethanol
6,7-dimethoxy-2-methyl-N-[(1R)-1-(3-{1-[2-(pyrrolidin-1-yl)ethyl]-1H-pyrazol-4-yl}phenyl)ethyl]quinazolin-4-amine
N-{(1R)-1-[2′-(aminomethyl)biphenyl-3-yl]ethyl}-6,7-dimethoxy-2-methylquinazolin-4-amine
N-[1-{5-[2-(aminomethyl)phenyl]thiophen-3-yl}ethyl]-6,7-dimethoxy-2-methylquinazolin-4-amine
N-{(1R)-1-[3-(aminomethyl)phenyl]ethyl}-6,7-dimethoxy-2-methylquinazolin-4-amine
N-{(1R)-1-[3-(6,7-dihydro-5H-pyrrolo[1,2-a]imidazol-3-yl)phenyl]ethyl}-6,7-dimethoxy-2-methylquinazolin-4-amine
N-[1-(5-{2-[(dimethylamino)methyl]phenyl}thiophen-2-yl)ethyl]-6,7-dimethoxy-2-methylquinazolin-4-amine
6,7-dimethoxy-2-methyl-N-[1-(5-{2-[(methylamino)methyl]phenyl}thiophen-3-yl)ethyl]quinazolin-4-amine
N-[1-(4-bromothiophen-2-yl)ethyl]-6,7-dimethoxy-2-methylquinazolin-4-amine
N-[1-{5-[3-(aminomethyl)phenyl]thiophen-2-yl}ethyl]-6,7-dimethoxy-2-methylquinazolin-4-amine
N-[1-{5-[4-(aminomethyl)phenyl]thiophen-2-yl}ethyl]-6,7-dimethoxy-2-methylquinazolin-4-amine
6,7-dimethoxy-2-methyl-N-[1-(4-{1-[2-(pyrrolidin-1-yl)ethyl]-1H-pyrazol-3-yl}thiophen-2-yl)ethyl]quinazolin-4-amine
6,7-dimethoxy-2-methyl-N-[(1R)-1-{2′-[(methylamino)methyl]biphenyl-3-yl}ethyl]quinazolin-4-amine
N-[1-{4-[2-(aminomethyl)-4-fluorophenyl]thiophen-2-yl}ethyl]-6,7-dimethoxy-2-methylquinazolin-4-amine
N-[1-{4-[2-(aminomethyl)phenyl]thiophen-2-yl}ethyl]-6,7-dimethoxy-2-methylquinazolin-4-amine
6,7-dimethoxy-2-methyl-N-[1-(4-{2-[(methylamino)methyl]phenyl}thiophen-2-yl)ethyl]quinazolin-4-amine
N-{1-[4-(6,7-dihydro-5H-pyrrolo[1,2-a]imidazol-3-yl)thiophen-2-yl]ethyl}-6,7-dimethoxy-2-methylquinazolin-4-amine
2-[3-(5-{1-[(6,7-dimethoxy-2-methylquinazolin-4-yl)amino]ethyl}thiophen-3-yl)-1H-pyrazol-1-yl]ethanol
N-{(1R)-1-[2′-(aminomethyl)-4′-fluorobiphenyl-3-yl]ethyl}-6,7-dimethoxy-2-methylquinazolin-4-amine
N-[1-{5-[5-(aminomethyl)furan-2-yl]thiophen-2-yl}ethyl]-6,7-dimethoxy-2-methylquinazolin-4-amine
N-{1-[5′-(aminomethyl)-2,2′-bithiophen-5-yl]ethyl}-6,7-dimethoxy-2-methylquinazolin-4-amine
2-{[2-(5-{1-[(6,7-dimethoxy-2-methylquinazolin-4-yl)amino]ethyl}thiophen-2-yl)benzyl]amino}-1-(1H-indol-3-yl)ethanone
3-amino-4-(5-{1-[(6,7-dimethoxy-2-methylquinazolin-4-yl)amino]ethyl}thiophen-2-yl)-1-benzothiophene-2-carboxamide
2-[2-(5-{1-[(6,7-dimethoxy-2-methylquinazolin-4-yl)amino]ethyl}thiophen-2-yl)benzyl]glycinamide
2-[2-(5-{1-[(6,7-dimethoxy-2-methylquinazolin-4-yl)amino]ethyl}thiophen-2-yl)benzyl]-N, N-dimethylglycinamide methyl N-[2-(5-{1-[(6,7-dimethoxy-2-methylquinazolin-4-yl)amino]ethyl}thiophen-2-yl)benzyl]glycinate
2-[2-(5-{1-[(6,7-dimethoxy-2-methylquinazolin-4-yl)amino]ethyl}thiophen-2-yl)benzyl]-N-methylglycinamide
2-[2-(5-{1-[(6,7-dimethoxy-2-methylquinazolin-4-yl)amino]ethyl}thiophen-2-yl)benzyl]-N-(2-methoxyethyl)glycinamide
N-benzyl-2-[2-(5-{1-[(6,7-dimethoxy-2-methylquinazolin-4-yl)amino]ethyl}thiophen-2-yl)benzyl]glycinamide
2-{[2-(5-{1-[(6,7-dimethoxy-2-methylquinazolin-4-yl)amino]ethyl}thiophen-2-yl)benzyl]amino}-1-(morpholin-4-yl)ethanone
3-(5-{1-[(6,7-dimethoxy-2-methylquinazolin-4-yl)amino]ethyl}thiophen-2-yl)-1,5-dimethyl-1H-pyrrole-2-carbonitrile
5-{1-[(6,7-dimethoxy-2-methylquinazolin-4-yl)amino]ethyl}-2,3′-bithiophene-4′-carbonitrile
N-[1-(5-{2-[(diethylamino)methyl]phenyl}thiophen-2-yl)ethyl]-6,7-dimethoxy-2-methylquinazolin-4-amine
2-[2-(5-{1-[(6,7-dimethoxy-2-methylquinazolin-4-yl)amino]ethyl}thiophen-2-yl)benzyl]-N-phenylglycinamide
1-[2-(5-{1-[(6,7-dimethoxy-2-methylquinazolin-4-yl)amino]ethyl}thiophen-2-yl)benzyl]piperidine-3-carboxamide
N-{1-[5-(2-{[(2,2-difluoroethyl)(methyl)amino]methyl}phenyl)thiophen-2-yl]ethyl}-6,7-dimethoxy-2-methylquinazolin-4-amine
N-[1-{5-[2-(aminomethyl)-5-chlorophenyl]thiophen-2-yl}ethyl]-6,7-dimethoxy-2-methylquinazolin-4-amine
N-[2-(5-{1-[(6,7-dimethoxy-2-methylquinazolin-4-yl)amino]ethyl}thiophen-2-yl)-5-fluorobenzyl]-1H-imidazole-2-carboxamide
N-[2-(5-{1-[(6,7-dimethoxy-2-methylquinazolin-4-yl)amino]ethyl}thiophen-2-yl)-5-fluorobenzyl]-1H-imidazole-5-carboxamide
N-[2-(5-{1-[(6,7-dimethoxy-2-methylquinazolin-4-yl)amino]ethyl}thiophen-2-yl)-5-fluorobenzyl]-N&lt;sup&gt;2&lt;/sup&gt;-(2,2,2-trifluoroethyl)glycinamide
N-[2-(5-{1-[(6,7-dimethoxy-2-methylquinazolin-4-yl)amino]ethyl}thiophen-2-yl)-5-fluorobenzyl]-1H-indole-2-carboxamide
2-{[2-(5-{1-[(6,7-dimethoxy-2-methylquinazolin-4-yl)amino]ethyl}thiophen-2-yl)benzyl]amino}ethanol
2-{[2-(5-{1-[(6,7-dimethoxy-2-methylquinazolin-4-yl)amino]ethyl}thiophen-2-yl)benzyl](methyl)amino}ethanol
N-[2-(5-{1-[(6,7-dimethoxy-2-methylquinazolin-4-yl)amino]ethyl}thiophen-2-yl)-5-fluorobenzyl]-N2-phenylglycinamide
6,7-dimethoxy-2-methyl-N-{1-[5-(2-{[(2,2,2-trifluoroethyl)amino]methyl}phenyl)thiophen-2-yl]ethyl}quinazolin-4-amine
6,7-dimethoxy-2-methyl-N-[1-(5-{2-[(pyridin-2-ylamino)methyl]phenyl}thiophen-2-yl)ethyl]quinazolin-4-amine
6,7-dimethoxy-2-methyl-N-[1-(5-{2-[(1H-pyrazol-3-ylamino)methyl]phenyl}thiophen-2-yl)ethyl]quinazolin-4-amine
1-(3,4-dihydroisoquinolin-2(1H)-yl)-2-{[2-(5-{1-[(6,7-dimethoxy-2-methylquinazolin-4-yl)amino]ethyl}thiophen-2-yl)benzyl]amino}ethanone
N-[1-{5-[4-fluoro-2-({[(1-methyl-1H-imidazol-2-yl)methyl]amino}methyl)phenyl]thiophen-2-yl}ethyl]-6,7-dimethoxy-2-methylquinazolin-4-amine
6,7-dimethoxy-2-methyl-N-[1-{5-[2-(piperazin-1-ylmethyl)phenyl]thiophen-2-yl}ethyl]quinazolin-4-amine hydrochloride
tert-butyl 4-[2-(5-{1-[(6,7-dimethoxy-2-methylquinazolin-4-yl)amino]ethyl}thiophen-2-yl)benzyl]piperazine-1-carboxylate
N-[2-(5-{1-[(6,7-dimethoxy-2-methylquinazolin-4-yl)amino]ethyl}thiophen-2-yl)-5-fluorobenzyl]acetamide
6,7-dimethoxy-2-methyl-N-[1-(5-{2-[(4-methylpiperazin-1-yl)methyl]phenyl}thiophen-2-yl)ethyl]quinazolin-4-amine
(3S)-{[2-(5-{1-[(6,7-dimethoxy-2-methylquinazolin-4-yl)amino]ethyl}thiophen-2-yl)benzyl]amino}-1-methylpyrrolidin-2-one
N-[2-(5-{1-[(6,7-dimethoxy-2-methylquinazolin-4-yl)amino]ethyl}thiophen-2-yl)-5-fluorobenzyl]-1H-pyrazole-3-carboxamide
6,7-dimethoxy-2-methyl-N-[1-{5-[2-(morpholin-4-ylmethyl)phenyl]thiophen-2-yl}ethyl]quinazolin-4-amine
1-[2-(5-{1-[(6,7-dimethoxy-2-methylquinazolin-4-yl)amino]ethyl}thiophen-2-yl)benzyl]azetidin-3-ol
2-[2-(5-{1-[(6,7-dimethoxy-2-methylquinazolin-4-yl)amino]ethyl}thiophen-2-yl)benzyl]-2,5,7-triazaspiro[3.4]octan-6-one
N-[2-(5-{1-[(6,7-dimethoxy-2-methylquinazolin-4-yl)amino]ethyl}thiophen-2-yl)-5-fluorobenzyl]-L-prolinamide
N-{1-[5-(2-{[(2,2-difluoroethyl)amino]methyl}phenyl)thiophen-2-yl]ethyl}-6,7-dimethoxy-2-methylquinazolin-4-amine
N-[2-(5-{1-[(6,7-dimethoxy-2-methylquinazolin-4-yl)amino]ethyl}thiophen-2-yl)-5-fluorobenzyl]-prolinamide
N-[1-{5-[2-(azetidin-1-ylmethyl)phenyl]thiophen-2-yl}ethyl]-6,7-dimethoxy-2-methylquinazolin-4-amine
{1-[(2S)-(5-{1-[(6,7-dimethoxy-2-methylquinazolin-4-yl)amino]ethyl}thiophen-2-yl)benzyl]azetidin-2-yl}methanol
N-{1-[5-(2-{[3-(dimethylamino)azetidin-1-yl]methyl}phenyl)thiophen-2-yl]ethyl}-6,7-dimethoxy-2-methylquinazolin-4-amine
N-[1-(5-{2-[(3,3-difluoroazetidin-1-yl)methyl]phenyl}thiophen-2-yl)ethyl]-6,7-dimethoxy-2-methylquinazolin-4-amine
6,7-dimethoxy-2-methyl-N-{1-[5-(2-{[methyl(2,2,2-trifluoroethyl)amino]methyl}phenyl)thiophen-2-yl]ethyl}quinazolin-4-amine
N-[1-(5-{2-[(3-fluoroazetidin-1-yl)methyl]phenyl}thiophen-2-yl)ethyl]-6,7-dimethoxy-2-methylquinazolin-4-amine
N-[1-(5-{4-chloro-2-[(dimethylamino)methyl]phenyl}thiophen-2-yl)ethyl]-6,7-dimethoxy-2-methylquinazolin-4-amine
1-[2-(5-{1-[(6,7-dimethoxy-2-methylquinazolin-4-yl)amino]ethyl}thiophen-2-yl)phenyl]ethanone
6,7-dimethoxy-2-methyl-N-[1-(5-{2-[2-(pyrrolidin-1-yl)ethoxy]phenyl}thiophen-2-yl)ethyl]quinazolin-4-amine
6,7-dimethoxy-2-methyl-N-[1-(4-{2-[(methylamino)methyl]phenyl}thiophen-2-yl)ethyl]quinazolin-4-amine, enantiomer 1
6,7-dimethoxy-2-methyl-N-[1-(4-{2-[(methylamino)methyl]phenyl}thiophen-2-yl)ethyl]quinazolin-4-amine, enantiomer 2
N-{1-[5-(6,7-dihydro-5H-pyrrolo[1,2-a]imidazol-3-yl)thiophen-2-yl]ethyl}-6,7-dimethoxy-2-methylquinazolin-4-amine, enantiomer 1
N-{1-[5-(6,7-dihydro-5H-pyrrolo[1,2-a]imidazol-3-yl)thiophen-2-yl]ethyl}-6,7-dimethoxy-2-methylquinazolin-4-amine, enantiomer 2 6,7-dimethoxy-2-methyl-N-[1-(2-methyl-1-benzothiophen-4-yl)ethyl]quinazolin-4-amine
6,7-dimethoxy-2-methyl-N-[1-(thieno[2,3-b]pyridin-4-yl)ethyl]quinazolin-4-amine
6,7-dimethoxy-2-methyl-N-[1-(thieno[2,3-c]pyridin-4-yl)ethyl]quinazolin-4-amine
6,7-dimethoxy-2-methyl-N-[1-(thieno[3,2-c]pyridin-4-yl)ethyl]quinazolin-4-amine
N-{(1R)-1-[3-(3,5-dimethyl-1H-pyrazol-4-yl)phenyl]ethyl}-6,7-dimethoxy-2-methylquinazolin-4-amine
6,7-dimethoxy-2-methyl-N-{(1R)-1-[3-(5-methyl-1H-pyrazol-4-yl)phenyl]ethyl}quinazolin-4-amine
N-{(1R)-1-[3-(3,5-dimethyl-1,2-oxazol-4-yl)phenyl]ethyl}-6,7-dimethoxy-2-methylquinazolin-4-amine
6,7-dimethoxy-2-methyl-N-{(1R)-1-[3-(1H-pyrazol-5-yl)phenyl]ethyl}quinazolin-4-amine
6,7-dimethoxy-2-methyl-N-{(1R)-1-[3-(1-methyl-1H-pyrazol-4-yl)phenyl]ethyl}quinazolin-4-amine
6,7-dimethoxy-2-methyl-N-{(1R)-1-[3-(1-methyl-1H-pyrazol-5-yl)phenyl]ethyl}quinazolin-4-amine
N-{(1R)-1-[3-(1H-imidazol-1-yl)phenyl]ethyl}-6,7-dimethoxy-2-methylquinazolin-4-amine
6,7-dimethoxy-2-methyl-N-{(1R)-1-[3-(1H-pyrazol-1-yl)phenyl]ethyl}quinazolin-4-amine
N-{(1R)-1-[3-(1H-imidazol-4-yl)phenyl]ethyl}-6,7-dimethoxy-2-methylquinazolin-4-amine
6-(benzyloxy)-N-[(1R)-1-(3-bromophenyl)ethyl]-7-methoxy-2-methylquinazolin-4-amine
6-(benzyloxy)-7-methoxy-2-methyl-N-{(1R)-1-[3-(1H-pyrazol-4-yl)phenyl]ethyl}quinazolin-4-amine
7-methoxy-2-methyl-4-({(1R)-1-[3-(1H-pyrazol-4-yl)phenyl]ethyl}amino)quinazolin-6-ol
6-(cyclopropylmethoxy)-7-methoxy-2-methyl-N-{(1R)-1-[3-(1H-pyrazol-4-yl)phenyl]ethyl}quinazolin-4-amine
6,7-dimethoxy-2-methyl-N-[1-(thiophen-2-yl)ethyl]quinazolin-4-amine
7-methoxy-6-(2-methoxyethoxy)-2-methyl-N-{(1R)-1-[3-(1H-pyrazol-4-yl)phenyl]ethyl}quinazolin-4-amine
(1R)-1-[2-(5-{1-[(6,7-dimethoxy-2-methylquinazolin-4-yl)amino]ethyl}thiophen-2-yl)phenyl]propan-1-ol
6-butoxy-7-methoxy-2-methyl-N-{(1R)-1-[3-(1H-pyrazol-4-yl)phenyl]ethyl}quinazolin-4-amine
7-methoxy-2-methyl-6-(3-methylbutoxy)-N-{(1R)-1-[3-(1H-pyrazol-4-yl)phenyl]ethyl}quinazolin-4-amine
tert-butyl {2-[2-(5-{1-[(6,7-dimethoxy-2-methylquinazolin-4-yl)amino]ethyl}thiophen-2-yl)phenyl]ethyl}carbamate
7-methoxy-2-methyl-6-(propan-2-yloxy)-N-{(1R)-1-[3-(1H-pyrazol-4-yl)phenyl]ethyl}quinazolin-4-amine
7-methoxy-2-methyl-6-(oxetan-3-ylmethoxy)-N-{(1R)-1-[3-(1H-pyrazol-4-yl)phenyl]ethyl}quinazolin-4-amine
6-ethoxy-7-methoxy-2-methyl-N-{(1R)-1-[3-(1H-pyrazol-4-yl)phenyl]ethyl}quinazolin-4-amine
6-ethoxy-N-{(1R)-1-[3-(1-ethyl-1H-pyrazol-4-yl)phenyl]ethyl}-7-methoxy-2-methylquinazolin-4-amine
N-[1-{5-[2-(2-aminoethyl)phenyl]thiophen-2-yl}ethyl]-6,7-dimethoxy-2-methylquinazolin-4-amine
tert-butyl {1-[2-(5-{1-[(6,7-dimethoxy-2-methylquinazolin-4-yl)amino]ethyl}thiophen-2-yl)phenyl]ethyl}carbamate
N-[1-(5-{2-[1-aminoethyl]phenyl}thiophen-2-yl)ethyl]-6,7-dimethoxy-2-methylquinazolin-4-amine
N-[1-(5-{2-[1-aminoethyl]phenyl}thiophen-2-yl)ethyl]-6,7-dimethoxy-2-methylquinazolin-4-amine
6,7-dimethoxy-2-methyl-N-[1-{5-[2-(1H-pyrazol-4-yl)phenyl]thiophen-2-yl}ethyl]quinazolin-4-amine
6,7-dimethoxy-2-methyl-N-[1-(5-{2-[(phenylamino)methyl]phenyl}thiophen-2-yl)ethyl]quinazolin-4-amine
6-bromo-N-[(1R)-1-(3-chlorophenyl)ethyl]-2-methylquinazolin-4-amine
N-[1-(5-{2-[(cyclopentylamino)methyl]phenyl}thiophen-2-yl)ethyl]-6,7-dimethoxy-2-methylquinazolin-4-amine
N-[1-(5-{2-[(benzylamino)methyl]phenyl}thiophen-2-yl)ethyl]-6,7-dimethoxy-2-methylquinazolin-4-amine
N-[1-(5-{2-[(butylamino)methyl]phenyl}thiophen-2-yl)ethyl]-6,7-dimethoxy-2-methylquinazolin-4-amine
N-[1-(5-{2-[(ethylamino)methyl]phenyl}thiophen-2-yl)ethyl]-6,7-dimethoxy-2-methylquinazolin-4-amine
6,7-dimethoxy-2-methyl-N-[1-{5-[2-(1H-tetrazol-5-yl)phenyl]thiophen-2-yl}ethyl]quinazolin-4-amine
6,7-dimethoxy-N-{1-[5-(2-{[(2-methoxyethyl)amino]methyl}phenyl)thiophen-2-yl]ethyl}-2-methylquinazolin-4-amine
N-[1-(5-{2-[(cyclopropylamino)methyl]phenyl}thiophen-2-yl)ethyl]-6,7-dimethoxy-2-methylquinazolin-4-amine
methyl 4-{[(1R)-1-(3-chlorophenyl)ethyl]amino}-2-methylquinazoline-6-carboxylate
4-{[(1R)-1-(3-chlorophenyl)ethyl]amino}-2-methylquinazoline-6-carboxylic acid
(4-{[(1R)-1-(3-chlorophenyl)ethyl]amino}-2-methylquinazolin-6-yl)methanol
[2-(5-{1-[(6,7-dimethoxy-2-methylquinazolin-4-yl)amino]ethyl}thiophen-2-yl)phenyl](phenyl)methanol
1-[2-(5-{1-[(6,7-dimethoxy-2-methylquinazolin-4-yl)amino]ethyl}thiophen-2-yl)phenyl]-3-phenylpropan-1-ol
1-[2-(5-{1-[(6,7-dimethoxy-2-methylquinazolin-4-yl)amino]ethyl}thiophen-2-yl)phenyl]-2-phenylethanol
1-[2-(5-{1-[(6,7-dimethoxy-2-methylquinazolin-4-yl)amino]ethyl}thiophen-2-yl)phenyl]pentan-1-ol
1-[2-(5-{1-[(6,7-dimethoxy-2-methylquinazolin-4-yl)amino]ethyl}thiophen-2-yl)phenyl]prop-2-yn-1-ol
1-[2-(5-{1-[(6,7-dimethoxy-2-methylquinazolin-4-yl)amino]ethyl}thiophen-2-yl)phenyl]-2-methylpropan-1-ol
1-[2-(5-{1-[(6,7-dimethoxy-2-methylquinazolin-4-yl)amino]ethyl}thiophen-2-yl)phenyl]-2,2,2-trifluoroethanol
N-{1-[5-(6,7-dihydro-5H-pyrrolo[1,2-a]imidazol-3-yl)-4-methylthiophen-2-yl]ethyl}-6,7-dimethoxy-2-methylquinazolin-4-amine
N-[2-(5-{1-[(6,7-dimethoxy-2-methylquinazolin-4-yl)amino]ethyl}thiophen-2-yl)-5-fluorobenzyl]-2-hydroxyacetamide
N-[2-(5-{1-[(6,7-dimethoxy-2-methylquinazolin-4-yl)amino]ethyl}thiophen-2-yl)-5-fluorobenzyl]-2-methoxyacetamide
N-(1-(5-(4-bromo-2-((dimethylamino)methyl)phenyl)thiophen-2-yl)ethyl)-6,7-dimethoxy-2-methylquinazolin-4-amine
N-(1-(5-(2-((Dimethylamino)methyl)-4-(trifluoromethyl)phenyl) thiophen-2-yl) ethyl)-6,7-dimethoxy-2-methylquinazolin-4-amine
6,7-dimethoxy-2-methyl-N-(1-{5-[2-methyl-4-(trifluoromethyl)phenyl]-2-thienyl}ethyl)quinazolin-4-amine
tert-butyl [4-chloro-2-(5-{1-[(6,7-dimethoxy-2-methylquinazolin-4-yl)amino]ethyl}thiophen-2-yl)benzyl]carbamate
4-(3-{(1R)-1-[(6,7-dimethoxy-2-methylquinazolin-4-yl)amino]ethyl}phenyl)pyridin-2-ol
N-{1-[3-(benzyloxy)phenyl]ethyl}-6,7-dimethoxy-2-methylquinazolin-4-amine
N-[1-(5-{2-[(dimethylamino)methyl]phenyl}thiophen-2-yl)ethyl]-6,7-dimethoxy-2-methylquinazolin-4-amine (enantiomer 1)
N-[1-(5-{2-[(dimethylamino)methyl]phenyl}thiophen-2-yl)ethyl]-6,7-dimethoxy-2-methylquinazolin-4-amine (enantiomer 2)
2-(4-{[(1R)-1-(3-chlorophenyl)ethyl]amino}-2-methylquinazolin-6-yl)propan-2-ol
2-(3′-{(1R)-1-[(6,7-dimethoxy-2-methylquinazolin-4-yl)amino]ethyl}biphenyl-2-yl)acetamide
2-[2-(5-{1-[(6,7-dimethoxy-2-methylquinazolin-4-yl)amino]ethyl}thiophen-2-yl)phenyl]acetamide
5-(5-{1-[(6,7-dimethoxy-2-methylquinazolin-4-yl)amino]ethyl}thiophen-2-yl)pyridin-2-ol
N-[(1R)-1-(3-chlorophenyl)ethyl]-6-methoxy-2,8-dimethylquinazolin-4-amine
N-[1-{5-[2-(aminomethyl)-3-chlorophenyl]thiophen-2-yl}ethyl]-6,7-dimethoxy-2-methylquinazolin-4-amine
N-[1-{5-[2-(aminomethyl)-4-chlorophenyl]thiophen-2-yl}ethyl]-6,7-dimethoxy-2-methylquinazolin-4-amine
N-(1-{5-[2-(aminomethyl)-4-fluorophenyl]-4-methyl-2-thienyl}ethyl)-6,7-dimethoxy-2-methylquinazolin-4-amine
N-[1-(5-{2-[(dimethylamino)methyl]phenyl}-4-methyl-2-thienyl)ethyl]-6,7-dimethoxy-2-methylquinazolin-4-amine
2-{[2-(5-{1-[(6,7-dimethoxy-2-methylquinazolin-4-yl)amino]ethyl}-3-methyl-2-thienyl)benzyl](methyl)amino}ethanol
6,7-dimethoxy-2-methyl-N-[1-(4-methyl-5-{2-[(methylamino)methyl]-phenyl}thiophen-2-yl)ethyl]quinazolin-4-amine
1-[2-(5-{1-[(6,7-dimethoxy-2-methylquinazolin-4-yl)amino]ethyl}thiophen-2-yl) phenyl]ethane-1,2-diol
N-[(1R)-1-(3-chlorophenyl)ethyl]-2,6-dimethylquinazolin-4-amine
N-[(1R)-1-(3-chlorophenyl)ethyl]-2-methyl-6-(1H-pyrazol-4-yl)quinazolin-4-amine
N-[(1R)-1-(3-chlorophenyl)ethyl]-2-methyl-6-(1-methyl-1H-pyrazol-4-yl)quinazolin-4-amine
N-[(1R)-1-(3-chlorophenyl)ethyl]-6-cyclopropyl-2-methylquinazolin-4-amine
tert-butyl [3-chloro-2-(5-{1-[(6,7-dimethoxy-2-methylquinazolin-4-yl)amino]ethyl}thiophen-2-yl)benzyl]carbamate
N-[1-{5-[2-(aminomethyl)-6-chlorophenyl]thiophen-2-yl}ethyl]-6,7-dimethoxy-2-methylquinazolin-4-amine
4-(5-{1-[(6,7-dimethoxy-2-methylquinazolin-4-yl)amino]ethyl}-2-thienyl)pyridin-2-ol
4-[2-(5-{1-[(6,7-dimethoxy-2-methylquinazolin-4-yl)amino]ethyl}thiophen-2-yl)phenyl]azetidin-2-one
N-[(1R)-1-(3-chlorophenyl)ethyl]-6-methoxy-2,7-dimethylquinazolin-4-amine
4-(5-{1-[(6,7-dimethoxy-2-methylquinazolin-4-yl)amino]ethyl}thiophen-2-yl)-3-[(dimethylamino)methyl]benzonitrile
N-[1-(5-bromothiophen-2-yl)ethyl]-6-[3-(dimethylamino) pyrrolidin-1-yl]-2-methylquinazolin-4-amine
N-[1-(5-{2-[(dimethylamino)methyl]phenyl}thiophen-2-yl)ethyl]-2-methyl-6-(pyrrolidin-1-yl)quinazolin-4-amine
N-[1-(5-bromothiophen-2-yl)ethyl]-2-methyl-6-(4-methylpiperazin-1-yl)quinazolin-4-amine
N-[1-(5-{2-[(dimethylamino)methyl]phenyl}thiophen-2-yl)ethyl]-2-methyl-6-(4-methylpiperazin-1-yl)quinazolin-4-amine
N-[1-(5-{2-[(dimethylamino)methyl]phenyl}thiophen-2-yl)ethyl]-6-[3-(dimethylamino)pyrrolidin-1-yl]-2-methylquinazolin-4-amine
N-[(1R)-1-(5-bromothiophen-2-yl)ethyl]-2-methyl-6-(pyrrolidin-1-yl)quinazolin-4-amine
N-(4-{[1-(5-{2-[(dimethylamino)methyl]phenyl}thiophen-2-yl)ethyl]amino}-2-methylquinazolin-6-yl)acetamide
N-[1-(5-{2-[(dimethylamino)methyl]phenyl}thiophen-2-yl)ethyl]-2-methyl-6-nitroquinazolin-4-amine
6,7-dimethoxy-N-{1-[5-(4-methoxy-2-methylphenyl)thiophen-2-yl]ethyl}-2-methylquinazolin-4-amine
N4-[1-(5-{2-[(dimethylamino)methyl]phenyl}-2-thienyl)ethyl]-2-methyl-quinazoline-4,6-diamine
N-[1-(5-bromothiophen-2-yl)ethyl]-2-methyl-6-[4-(pyridin-3-ylmethyl)piperazin-1-yl]quinazolin-4-amine
N4-[1-(5-bromo-2-thienyl)ethyl]-2-methyl-N6-[2-(morpholin-4-yl)ethyl]-quinazoline-4,6-diamine
N-[1-(5-{2-[(dimethylamino)methyl]phenyl}thiophen-2-yl)ethyl]-2-methyl-6-[4-(pyridin-3-ylmethyl)piperazin-1-yl]quinazolin-4-amine
N-{2-[(4-{[1-(5-{2-[(dimethylamino)methyl]phenyl}thiophen-2-yl)ethyl]amino}-2-methylquinazolin-6-yl)amino]ethyl}acetamide
N-[1-(5-bromo-3-chlorothiophen-2-yl)ethyl]-6,7-dimethoxy-2-methylquinazolin-4-amine
tert-butyl [2-(4-chloro-5-{1-[(6,7-dimethoxy-2-methylquinazolin-4-yl)amino]ethyl}thiophen-2-yl)benzyl]carbamate
N-[1-{5-[2-(aminomethyl)phenyl]-4-chlorothiophen-2-yl}ethyl]-6,7-dimethoxy-2-methylquinazolin-4-amine
N-[1-(4-chloro-5-{2-[(dimethylamino)methyl]phenyl}thiophen-2-yl)ethyl]-6,7-dimethoxy-2-methylquinazolin-4-amine
N-[1-(5-bromo-4-chlorothiophen-2-yl)ethyl]-6,7-dimethoxy-2-methylquinazolin-4-amine
N4-[1-(5-{2-[(dimethylamino)methyl]phenyl}-2-thienyl)ethyl]-2-methyl-N6-[2-(morpholin-4-yl)ethyl]quinazoline-4,6-diamine
4-(4-{[1-(5-bromothiophen-2-yl)ethyl]amino}-2-methylquinazolin-6-yl)-1-methylpiperazin-2-one
4-(4-{[1-(5-{2-[(dimethylamino)methyl]phenyl}thiophen-2-yl)ethyl]amino}-2-methylquinazolin-6-yl)-1-methylpiperazin-2-one
methyl 2-[2-(5-{1-[(6,7-dimethoxy-2-methylquinazolin-4-yl)amino]ethyl}-thiophen-2-yl)phenyl]-2-methylpropanoate
N-[1-{5-[2-(aminomethyl)phenyl]-3-chlorothiophen-2-yl}ethyl]-6,7-dimethoxy-2-methylquinazolin-4-amine
N-(4-{[1-(5-{2-[(dimethylamino)methyl]phenyl}thiophen-2-yl)ethyl]amino}-2-methylquinazolin-6-yl)methanesulfonamide
N-[1-(5-{2-[(dimethylamino)methyl]-4-methoxyphenyl}thiophen-2-yl)ethyl]-6,7-dimethoxy-2-methylquinazolin-4-amine
3-(4-{[1-(5-{2-[(dimethylamino)methyl]phenyl}thiophen-2-yl)ethyl]amino}-2-methylquinazolin-6-yl)-1,1-dimethylurea
1-benzyl-4-(4-{[1-(5-bromothiophen-2-yl)ethyl]amino}-2-methylquinazolin-6-yl)piperazin-2-one
N-[1-(5-{2-[(dimethylamino)methyl]-4-methylphenyl}thiophen-2-yl)ethyl]-6,7-dimethoxy-2-methylquinazolin-4-amine
N-[1-(5-{4-cyclopropyl-2-[(dimethylamino)methyl]phenyl}thiophen-2-yl)ethyl]-6,7-dimethoxy-2-methylquinazolin-4-amine
6,7-dimethoxy-2-methyl-N-[1-(2-methyl-1,3-thiazol-4-yl)ethyl]quinazolin-4-amine
6,7-dimethoxy-2-methyl-N-[1-(4-methyl-1,3-thiazol-2-yl)ethyl]quinazolin-4-amine
3-(5-{1-[(6,7-dimethoxy-2-methylquinazolin-4-yl)amino]ethyl}thiophen-2-yl)-1-methyl-1H-pyrazole-5-carboxylic acid
tert-butyl [(5′-{1-[(6,7-dimethoxy-2-methylquinazolin-4-yl)amino]ethyl}-2,2′-bithiophen-5-yl)methyl]carbamate
7-methoxy-2-methyl-6-[2-(methylsulfonyl)ethoxy]-N-{(1R)-1-[3-(1H-pyrazol-4-yl)phenyl]ethyl}quinazolin-4-amine
tert-butyl [5-chloro-2-(5-{1-[(6,7-dimethoxy-2-methylquinazolin-4-yl)amino]ethyl}thiophen-2-yl)benzyl]carbamate
tert-butyl [2-chloro-6-(5-{1-[(6,7-dimethoxy-2-methylquinazolin-4-yl)amino]ethyl}thiophen-2-yl)benzyl]carbamate
7-bromo-N-[(1R)-1-(3-chlorophenyl)ethyl]-2-methylquinazolin-4-amine
N-[1-(5-bromothiophen-2-yl)ethyl]-2-methyl-6-nitroquinazolin-4-amine
methyl 4-{[(1R)-1-(3-chlorophenyl)ethyl]amino}-2-methylquinazoline-7-carboxylate
3-amino-3-[2-(5-{1-[(6,7-dimethoxy-2-methylquinazolin-4-yl)amino]ethyl}thiophen-3-yl)phenyl]propanoic acid
N-[1-{5-[2-(2-aminopropan-2-yl)phenyl]thiophen-2-yl}ethyl]-6,7-dimethoxy-2-methylquinazolin-4-amine
{[2-(5-{1-[(6,7-dimethoxy-2-methylquinazolin-4-yl)amino]ethyl}thiophen-2-yl)benzyl](methyl)amino}acetonitrile
1-(4-{[1-(5-{2-[(dimethylamino)methyl]phenyl}thiophen-2-yl)ethyl]amino}-2-methylquinazolin-6-yl)-3-methylurea and
1-benzyl-4-(4-{[1-(5-{2-[(dimethylamino)methyl]phenyl}thiophen-2-yl)ethyl]amino}-2-methylquinazolin-6-yl)piperazin-2-one

In another embodiment of the first aspect, the present invention covers compounds of formula (I), supra, in which the carbon atom between the nitrogen atom and the substituent A1 is in (R)-configuration.

In yet another embodiment of the first aspect, the present invention covers compounds of formula (I), supra, wherein R¹ is selected from the list of the following substituents H, *—OCH₃, *—OC₂H₅,

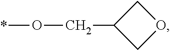 [SMILES: *OCC1COC1]

*—CH₂OH, *—C(O)OH, *—C(O)OCH₃, —Br, —O—CH(CH₃)₂, *—O—(CH₂)₂CH(CH₃)₂, *—O—(CH₂)₃CH₃, *—O—(CH₂)₂O—CH₃,

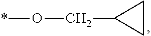 [SMILES: *OCC1CC1]

*—O—CH₂-Phenyl, *—N═S(O)(CH₃)₂, *—CH₃,

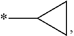 [SMILES: *C1CC1]

*—NH(CH₃), *—N(CH₃)₂, *—NH₂,

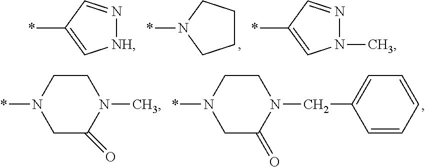

*—C(CH₃)₂—OH,

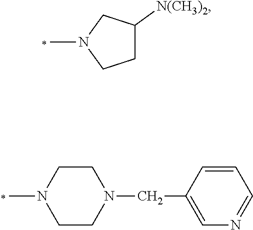

*—NH—(CH₂)₂—NH—C(O)—CH₃, *—NH—(CH₂)₂-morpholino, *—NH—C(O)—CH₃, *—NH—C(O)—NH—CH₃, *—NH—C(O)—N(CH₃)₂, *—NO₂, *—NH—S(O)₂—CH₃, *—N═S(O)(CH₃)₂, or *—OH, *—O—(CH₂)₂—S(O)₂—CH₃. *-fluorine,

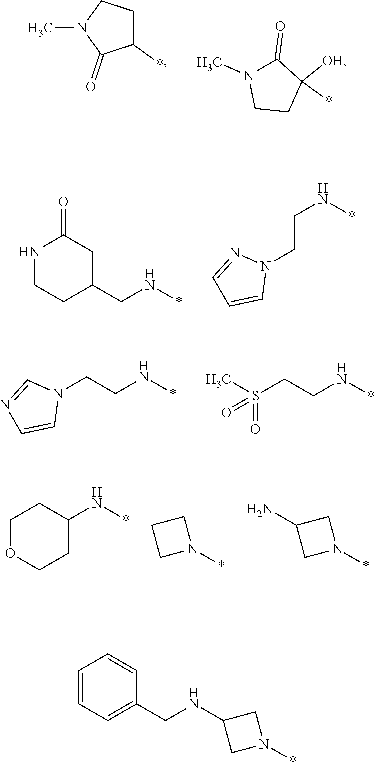

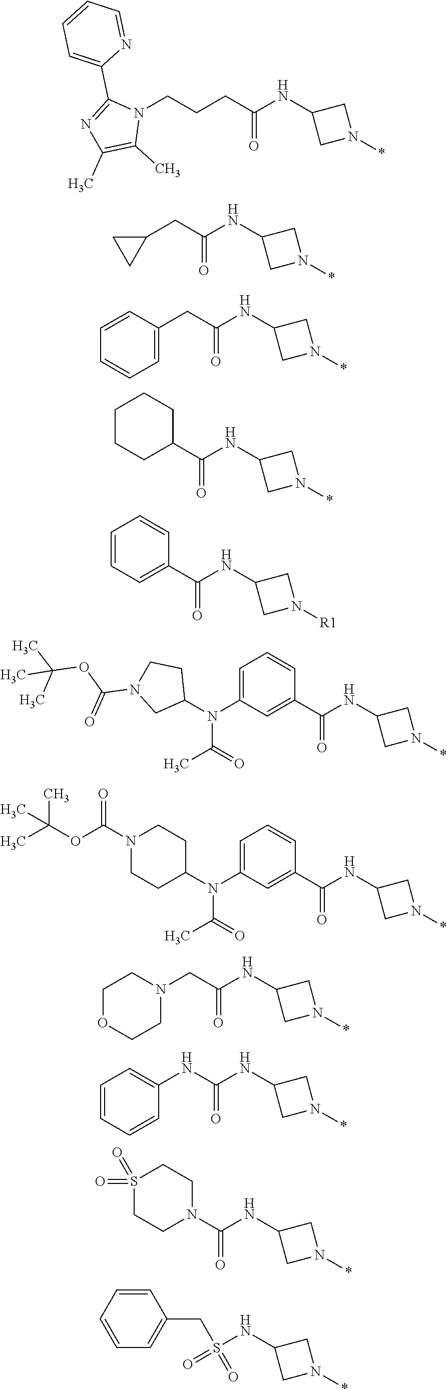

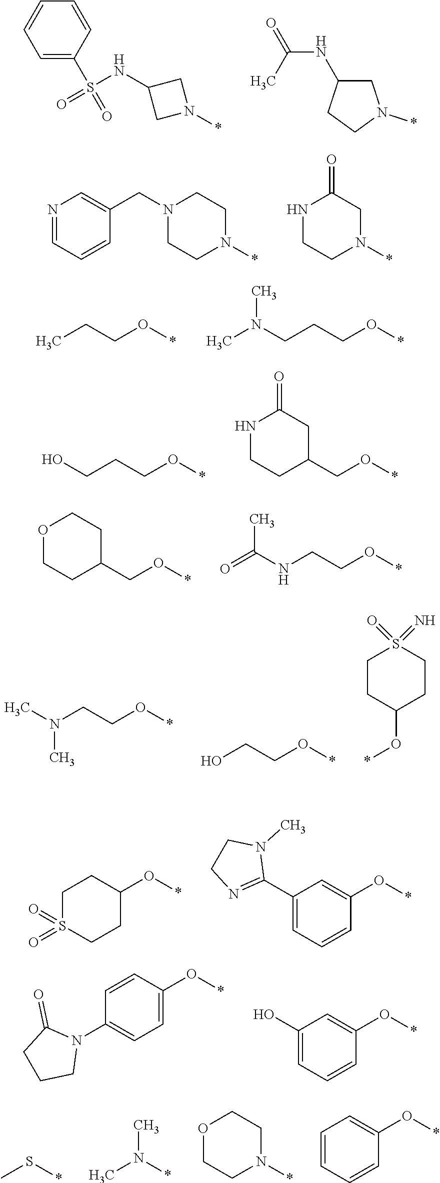

In a further embodiment of the first aspect, the present invention covers compounds of formula (I), supra, wherein R² is selected from the group of hydrogen, hydroxy, oxo (═O), cyano, cyclopropyl, 1,1-dimethylcyclopropyl, —C(═CH₂)CH₃, —C(CH₃)═CHCH₃, —CH═CH—(CH₂)₂CH₃, CH═CHCH₃, —CH═CH-cyclopropyl), —C(O)NH₂, C(O)OCH₃, —S(O)₂CH₃, —OCH₃, —CH₂NH₂, a halogen atom (F, Cl; Br),

In an even further embodiment of the first aspect, the present invention covers compounds of formula (I), supra, wherein A1 is selected from the group

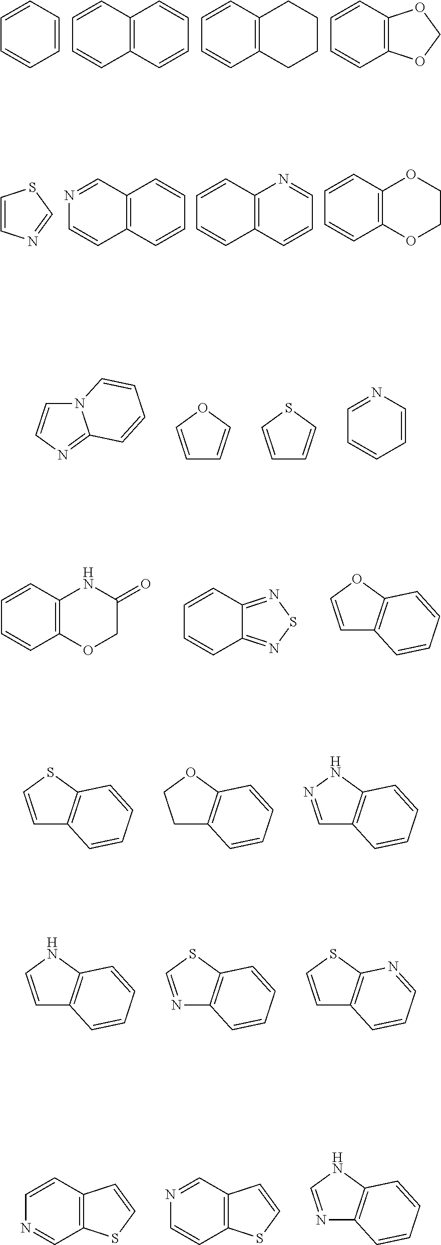

or a stereoisomer, a tautomer, an N-oxide, a hydrate, a solvate, or a salt thereof, or a mixture of same.

In a further another embodiment of the first aspect, the present invention covers compounds of formula (I), supra, wherein A1 is a phenyl ring or a thienyl ring.

In a particular embodiment of the first aspect, the present invention covers compounds of formula (I), supra, wherein A2 is selected from the group

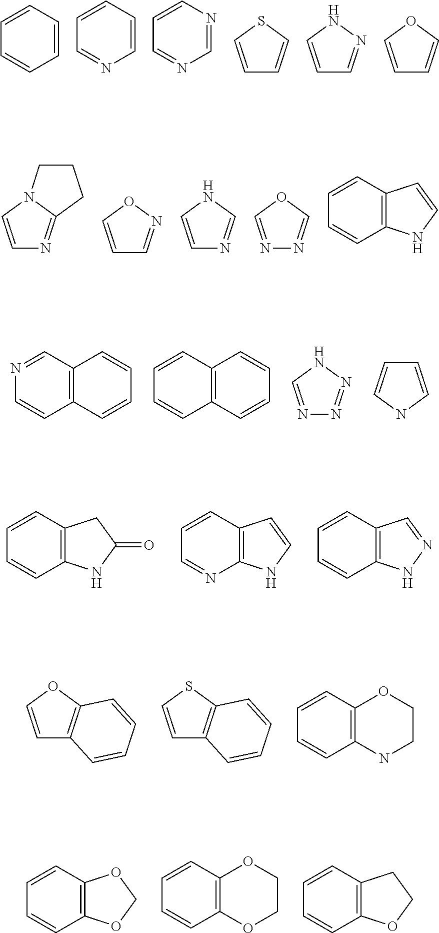

or a stereoisomer, a tautomer, an N-oxide, a hydrate, a solvate, or a salt thereof, or a mixture of same.

In a further particular embodiment of the first aspect, the present invention covers compounds of formula (I), supra, wherein A2 is a phenyl ring.

In another further particular embodiment of the first aspect, the present invention covers compounds of formula (I), supra, wherein R³ is selected from the group of the following substituents
*—C(O)NH—(CH₂)₂CH₃
*—C(O)—N(CH₃)₂
*—C(O)—NH₂
*—C(O)—NH—(CH₂)₂N(CH₃)₂
*—CH₂—C(O)—NH₂ hydrogen
*—F, *—Cl, *—Br
*—C≡N; *—CF₃, *—CH₃, *—C₂H₅, *—CH═CH₂;
*—CH₂—CN; *—CH(CH₃)—NH₂; *—CH═CH—CN;
*—O(O)—OH; *—C(O)—OCH₃; *—C(O)—CH₃; *—C(CH₃)₂—C(O)—OCH₃; *—C(CH₃)₂—CN; Oxo(=O);
hydroxy;

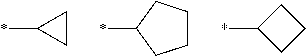

*—NH₂
*—NH—C(O)CH₃
*—NH—SO₂—CH₃
*—NH—C(O)—O—C(CH₃)₃

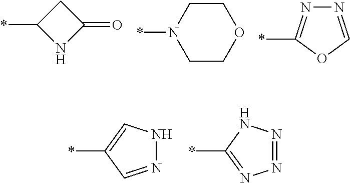

*—SO₂—CH₃
*—SO₂—N(CH₃)₂
*—SO₂—NH₂
*—O—CH₂—CH₃; *—O—(CH₂)₂—CH₃; *—O—CF₃;

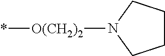

*—OCH₂-Cyclopropyl; *—OCH₃;
*—O(CH₂)₃—CH₃; *—OCH₂-Phenyl; *—O-Phenyl;
*—(CH₂)—OH
*—(CH₂)₂—OH
*—(CH₂)—O—CH₃
*—(CH₂)—O—CH₂—CH₃
*—CH(OH)—CH₂-Phenyl
*—CH(OH)—CH₂—CH₃
*—CH(OH)—(CH₂)₂—CH₃*—CH(OH)—(CH₂)₃—CH₃
*—CH(OH)—CH—(CH₃)₂
*—CH(OH)-Phenyl
*—CH(OH)—CN
*—CH(OH)—CH₂OH
*—CH(OH)—CF3
*—CH(OH)—(CH₂)₂-Phenyl
*—CH(OH)—C≡CH
*—CH(NH₂)—CH₂—COOH
*—CH₂—NH—SO₂—CH₃
*—CH₂—NH—(CH₂)₃—CH₃
*—CH₂—NH—CH₃
*—CH₂—N(CH₃)₂
*—CH₂—NH—C₂H5*—CH(CH₃)—NH₂
*—CH₂—NH₂
*—(CH₂)₂—NH₂
*—CH₂—NH—CH₂-Phenyl
*—CH₂—N(C₂H₅)₂
*—CH₂—NH-Cyclopropyl
*—CH₂—NH-Cyclobutyl
*—CH₂—NH-Cyclopentyl
*—CH₂—NH-Pyridyl
*—CH₂—NH-Phenyl
*—CH₂—NH—(CH₂)₂—OH
*—CH₂—N(CH₃)(CH₂)₂OH*—CH₂—NH—CH₂—CN
*—CH₂—N(CH₃)—CH₂—CN
*—CH₂—N(CH₃)—CH₂—CF₃
*—CH₂—N(CH₃)—CH₂—CF₂H
*—CH₂—NH—CH₂—CF₂H
*—CH₂—NH—CH₂—CF₃
*—CH₂—NH—(CH₂)₂—OCH₃

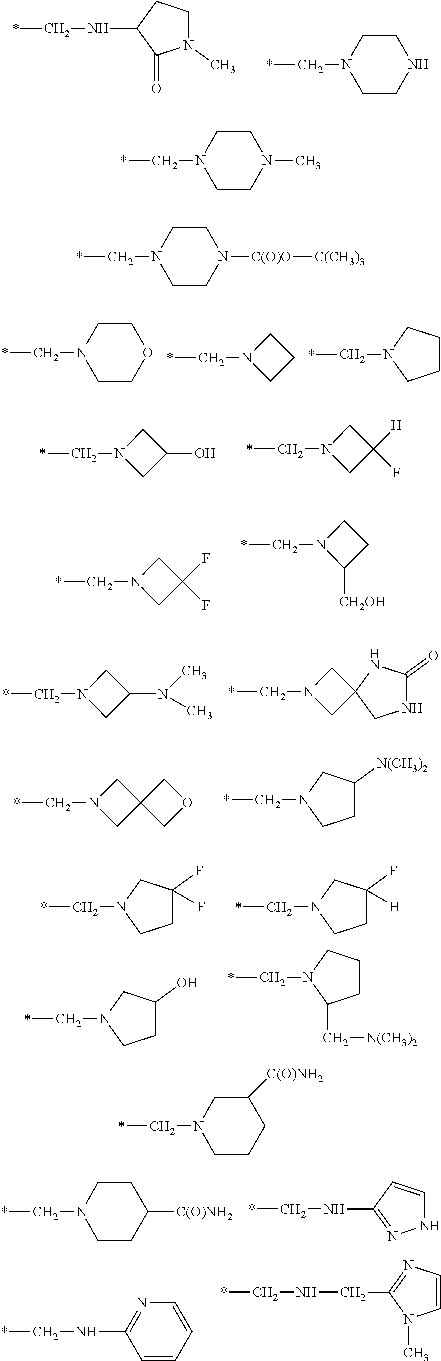

*—CH₂—NH—C(O)—O—C(CH₃)₃
*—(CH₂)₂—NH—C(O)—O—C(CH₃)₃
*—CH₂—NH—C(O)—CH₂—OH
*—CH₂—NH—C(O)—CH₂—OCH₃
*—CH—(CH₃)—NH—C(O)—O—C(CH₃)₃
*—CH₂—NH—C(O)—CH₃

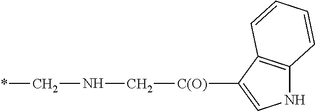

*—CH₂—NH—CH₂—C(O)—NH₂
*—CH₂—NH—CH₂—C(O)—N(CH₃)₂
*—CH₂—NH—CH₂—C(O)—OCH₃
*—CH₂—NH—CH₂—C(O)—NHCH₃
*—CH₂—NH—CH₂—C(O)—NH—(CH₂)₂—O—CH₃
*—CH₂—NH—CH₂—C(O)—NH—CH₂-Phenyl

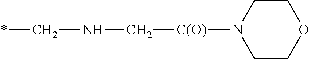

*—CH₂—NH—CH₂—C(O)—NH-Phenyl

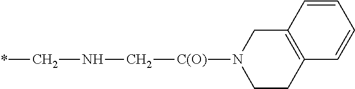

*—CH₂—NH—C(O)—CH₂—NH-Phenyl

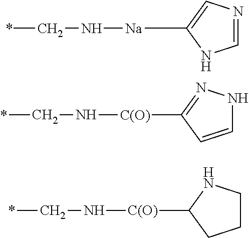

*—CH₂—NH—C(O)—CH₂—NH—CH₂—CF₃

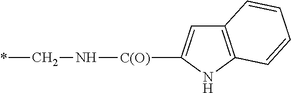

In yet another further particular embodiment of the first aspect, the present invention covers compounds of formula (I), supra, wherein R³ is a C₁- or C₂-alkyl substituted with an amino group —NR^{k}R^{l}, wherein R^{k} and R^{l} can have all the meanings as defined supra within the definition of R³ or wherein R³ is a C₁- or C₂-alkyl substituted with a hydroxyl or a C₁-C₆-alkoxy or a stereoisomer, a tautomer, an N-oxide, a hydrate, a solvate, or a salt thereof, or a mixture of same.

In even further particular embodiments of the first aspect, the present invention covers compounds of formula (I), supra, wherein x is 1 or 2 and/or y is 1 or 2 and/or z is 1 or 2 or a stereoisomer, a tautomer, an N-oxide, a hydrate, a solvate, or a salt thereof, or a mixture of same.

In a particular further embodiment of the first aspect, the present invention covers combinations of two or more of the above mentioned embodiments under the heading “further embodiments of the first aspect of the present invention”.

The present invention covers any sub-combination within any embodiment or aspect of the present invention of compounds of general formula (I), supra.

The present invention covers any sub-combination within any embodiment or aspect of the present invention of intermediate compounds of general formula (II). The present invention covers the compounds of general formula (I) which are disclosed in the Example Section of this text, infra.

### Synthesis of Compounds (Overview)

The compounds of the present invention can be prepared as described in the following section. The schemes and the procedures described below illustrate general synthetic routes to the compounds of general formula (I) of the invention and are not intended to be limiting. It is clear to the person skilled in the art that the order of transformations as exemplified in the schemes can be modified in various ways. The order of transformations exemplified in the schemes is therefore not intended to be limiting. In addition, interconversion of any of the substituents can be achieved before and/or after the exemplified transformations. These modifications can be such as the introduction of protecting groups, cleavage of protecting groups, exchange, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example P. G. M. Wuts and T. W. Greene in “Protective Groups in Organic Synthesis”, 4′″ edition, Wiley 2006). Specific examples are described in the subsequent paragraphs. Further, it is possible that two or more successive steps may be performed without work-up being performed between said steps, e.g. a “one-pot” reaction, as is well-known to the person skilled in the art.

The syntheses of the compounds of the present invention are preferably carried out according to the general synthetic sequence, shown in schemes 1-7.

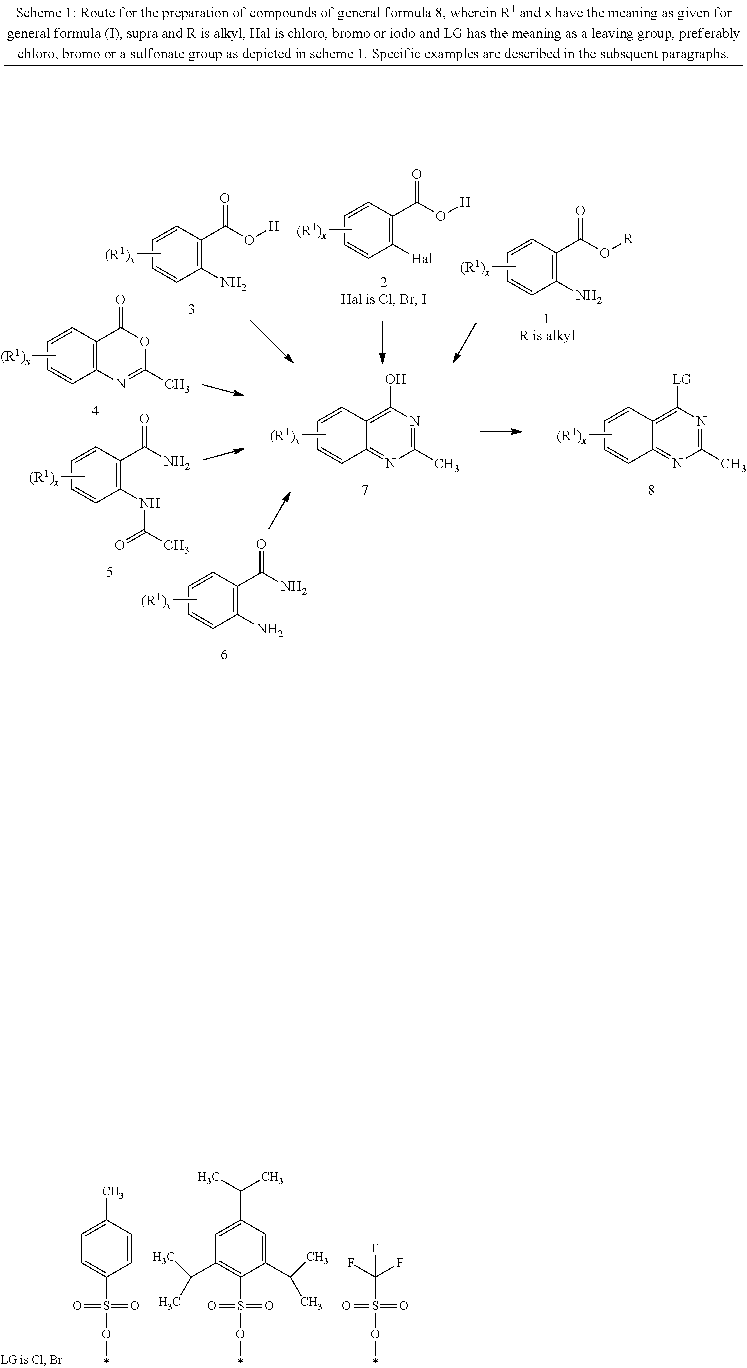

Step 1→7 (Scheme 1)

Quinazoline Formation

In the first step (scheme 1) amino benzoic acid ester derivative 1 (which is commercially available or described in the literature) can be converted to the corresponding quinazoline 7 in analogy to literature procedures. Typically acetonitrile and hydrochloric acid in organic solvent such as for example 1,4-dioxane at elevated temperatures is used. For example see ACS Medicinal Chemistry Letters, 2013, vol. 4, #9 p. 846-851; Journal of Medicinal Chemistry, 2009, vol. 52, #8 p. 2341-2351 or WO2015/54572 and references therein.

Step 2→7 (Scheme 1)

Quinazoline Formation

Alternatively halogen substituted benzoic acid derivative of general formula 2 (which is commercially available or described in the literature) can be converted to the corresponding quinazoline 7 in analogy to literature procedures. Typically derivative 2 is reacted with acetamidine, copper metal, a base such as for example potassium carbonate in an organic solvent such as for example DMF at elevated temperature. For example see WO2005/51410, US2008/107623 and references therein.

Step 3→7 (Scheme 1)

Quinazoline Formation

Alternatively amino substituted benzoic acid derivative of general formula 3 (which is commercially available or described in the literature) can be converted to the corresponding quinazoline 7 in analogy to literature procedures. Typically derivative 3 is reacted with acetyl chloride or acetic anhydride, an ammonia source such as for example ammonia or ammonium acetate, a base such as for example triethylamine or pyridine with or without DMAP in an organic solvent such as for example DMF, toluene, 1,4-dioxane/water at elevated temperature. For example see Bioorganic and Medicinal Chemistry Letters, 2011, vol. 21, #4 p. 1270-1274; Bioorganic and Medicinal Chemistry Letters, 2010, vol. 20, #7 p. 2330-2334; WO2008/117079 or WO2006/74187 and references therein.

Step 4→7 (Scheme 1)

Quinazoline Formation

Alternatively benzoxazinone derivative of general formula 4 (which is commercially available or can be prepared in analogy to literature procedures) can be converted to the corresponding quinazoline 7 in analogy to literature procedures. Typically derivative 4 is reacted with ammonium acetate in a solvent at elevated temperature. For example see Bioorganic and Medicinal Chemistry Letters, 2011, vol. 21, #4 p. 1270-1274 or U.S. Pat. No. 6,350,750 and references therein.

Step 5→7 (Scheme 1)

Quinazoline Formation

Alternatively benzoic acid amide derivative of general formula 5 (which is commercially available or described in the literature) can be converted to the corresponding quinazoline 7 in analogy to literature procedures. Typically derivative 5 is reacted with a base such as for example sodium hydroxide in a solvent such as for example water at elevated temperature.

For example, see Bioorganic and Medicinal Chemistry Letters, 2008, vol. 18, #16 p. 4573-4577 and references therein.

Step 6→7 (Scheme 1)

Quinazoline Formation

Alternatively amino benzoic acid amide derivative of general formula 6 (which is commercially available or described in the literature) can be converted to the corresponding quinazoline 7 in analogy to literature procedures. Typically derivative 6 is reacted with acetic acid at elevated temperature. For example see Bioorganic and Medicinal Chemistry Letters, 2008, vol. 18, #3 p. 1037-1041 and references therein.

Step 7→8 (Scheme 1)

Conversion of Hydroxyl Group into Leaving Group

In the next step (scheme 1) hydroxy quinazoline derivative 7 can be converted to the corresponding quinazoline 8 in analogy to literature procedures.

For W=chloro typically trichlorophosphate or thionylchloride, with or without N,N-dimethylaniline or N,N-diisopropylethylamine with or without an organic solvent such as for example toluene at elevated temperatures is used. For examples see Bioorganic and Medicinal Chemistry Letters, 2011, 1270; Journal of Medicinal Chemistry, 2009, 2341; ACS Medicinal Chemistry Letters, 2013, 846; Bioorganic and Medicinal Chemistry Letters, 2010, 2330; U.S. Pat. No. 6,350,750 or WO2015/54572 and references therein.

For W=bromo typically phosphorus oxytribromide, with or without N,N-dimethylaniline or N,N-diisopropylethylamine with or without an organic solvent such as for example toluene at elevated temperatures is used. For examples see US2012/53174; WO2012/30912 or WO2012/66122 and references therein.

For W=2,4,6-triisopropylsulfonate typically 2,4,6-triisopropylbenzenesulfonyl chloride, a base such as for example triethylamine and/or DMAP in an organic solvent such as for example dichloromethane is used. For examples see WO2010/99379 US2012/53176 and references therein.

For W=tosylate typically 4-methylbenzene-1-sulfonyl chloride, a base such as for example triethylamine or potassium carbonate and/or DMAP in an organic solvent such as for example dichloromethane or acetonitrile is used. For examples see Organic Letters, 2011, 4374 or Bioorganic and Medicinal Chemistry Letters, 2013, 2663 and references therein.

For W=trifluoromethanesulfonate typically N,N-bis(trifluoromethylsulfonyl)aniline or trifluoromethanesulfonic anhydride, a base such as for example triethylamine or 1,8-diazabicyclo[5.4.0]undec-7-ene and/or DMAP in an organic solvent such as for example dichloromethane is used. For examples see Journal of the American Chemical Society, 2015, 13433 or WO2014/100501 and references therein.

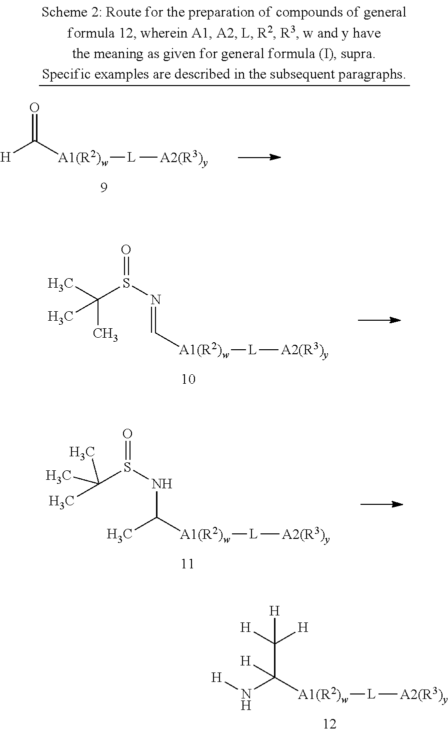

Step 9→10 (Scheme 2)

Sulfinimine Formation

In the first step (scheme 2) aldehyde derivative 9 (which is commercially available or described in the literature) can be converted to the corresponding sulfinimine 10 in analogy to the numerous literature procedures. For example the reaction can be performed at ambient temperature using Titanium(IV)ethoxide in an organic solvent as for example THF. For a review about sulfinimine chemistry see for example Chem. Rev. 2010, 110, 3600-3740; Chem. Soc. Rev. 2009, 38, 1162-1186; Tetrahedron 2004, 60, 8003 or WO2013030138 and the references therein.

Step 10→11 (Scheme 2)

Sulfinamide Formation

In the next step (scheme 2) sulfinimine 10 can be converted to the corresponding sulfinamide 11 in analogy to the numerous literature procedures. For example the reaction can be performed using methylmagnesium bromide in an organic solvent as for example THF. For a review about sulfinimine and sulfinamide chemistry see for example Chem. Rev. 2010, 110, 3600-3740; Chem. Soc. Rev. 2009, 38, 1162-1186; Tetrahedron 2004, 60, 8003 or WO2013030138 and the references therein.

Step 11→12 (Scheme 2)

Formation of Amine

In the next step (scheme 2) sulfinamide 11 can be converted to the corresponding amine 12 in analogy to the numerous literature procedures. For example the reaction can be performed using acetylchloride in a protic organic solvent as for example methanol. For a review about sulfinimine and sulfonamide chemistry see for example Chem. Rev. 2010, 110, 3600-3740; Chem. Soc. Rev. 2009, 38, 1162-1186; Tetrahedron 2004, 60, 8003 or WO2013030138 and the references therein.

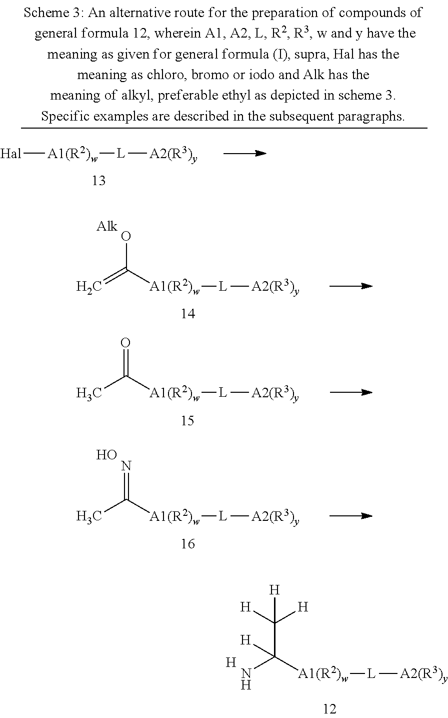

Step 13→14 (Scheme 3)

Stille Coupling

In the first step (scheme 3) halide derivative 13 (which is commercially available or described in the literature) can be converted to the corresponding enolester derivative 14 in analogy to literature procedures. Typically the reaction is performed with tributyl(1-ethoxyethenyl)stannane, a palladium catalyst such as for example bis-triphenylphosphine-palladium(II) chloride or dichloro(1,1′-bis(diphenylphosphanyl)ferrocene)palladium(II) dichloromethane adduct, with or without a base such as for example triethylamine in an organic solvent such as for example DMF, 1,4-dioxane or toluene at elevated temperature.

For W=bromo see for example the literature references WO2010/116282, WO2004/214, WO2013/185093 or Journal of the American Chemical Society, 2002, 6343 and references therein.

For W=chloro see for example the literature references Angewandte Chemie—International Edition, 1999, 2411-2413; Journal of the American Chemical Society, 2004, 16433, Organic Letters, 2004, 1421; Organic letters, 2001, 4295 and references therein.

For W=iodo see for example the literature references Bioorganic and Medicinal Chemistry Letters, 2003, 637, WO2011/100401, WO2007/38613 or US2005/143401 and references therein.

Step 14→15 (Scheme 3)

Formation of Methylketone

In the next step (scheme 3) enolester derivative 14 can be converted to the corresponding methyl ketone 15 in analogy to literature procedures. Typically the reaction is performed with an acid such as for example aqueous hydrochloric acid in an organic solvent such as for example THF, 1,4-dioxane or acetone. See for example the literature references Journal of Organic Chemistry, 1992, 1486, WO2013/185103 or U.S. Pat. No. 7,361,789 (2008) and references therein.

Step 15→16 (Scheme 3)

Formation of Oxime

In the next step (scheme 3) methyl ketone derivative 15 can be converted to the corresponding oxime 16 in analogy to literature procedures. Typically the reaction is performed with hydroxylamine hydrochloride with or without the addition of a base such as for example sodium acetate, pyridine, or KOH aq. in an organic solvent such as for example ethanol, DMSO, THF, dimethylether or methanol. See for example the literature references U.S. Pat. No. 5,332,757 (1994); US2004/157849 or International Journal of Pharmaceutics, 2016, 205 and references therein.

### Step 16→12 (Scheme 3)

Reduction of Oxime

In the next step (scheme 3) oxime derivative 16 can be reduced to the corresponding amine 12 in analogy to literature procedures. Typical reaction conditions include for example hydrogen, acetic acid, palladium on activated carbon in ethanol (see literature reference WO2006/82392 and references therein); ammonia, hydrogen, Raney nickel in methanol (see literature reference US2011/263626 (2011) and references therein); hydrogen, acetic acid, palladium on activated carbon in ethanol (see literature references WO2006/82392 and references therein) or acetic acid, zinc in methanol (see literature reference WO2013/26914 and references therein).

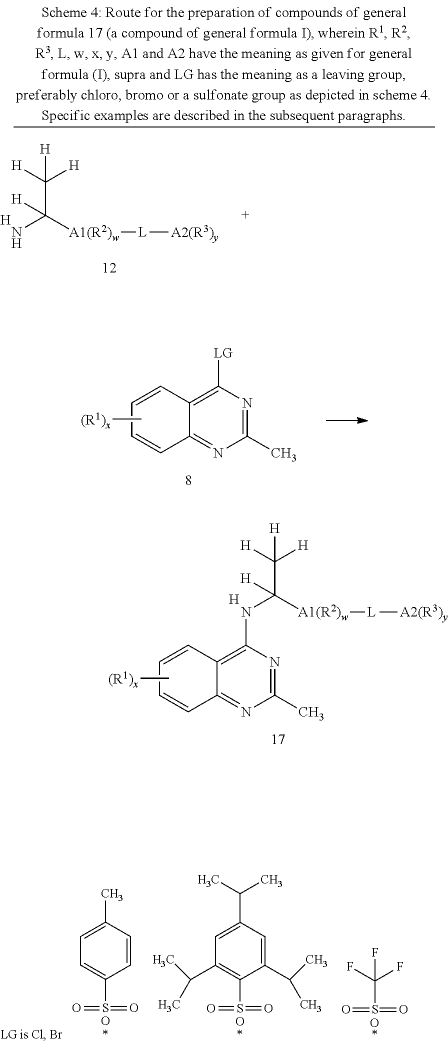

Step 12+8→17 (Scheme 4)

Amine Coupling

In the first step (scheme 4) amine derivative 12 and quinazoline derivative 8 are converted to amine 17 in analogy to literature procedures. Typically the reaction is performed in an organic solvent such as for example THF, DMF, acetonitrile dichloromethane or isopropyl alcohol with or without a base such as for example triethylamine, N-ethyl-N,N-diisopropylamine, potassium carbonate or potassium tert-butylate.

For LG=chloro see for example the literature references WO2008/86462; WO2008/86462 or European Journal of Medicinal Chemistry, 2015, 462 and references therein.

For LG=bromo see for example the literature references US2009/247519 or Journal of Organic Chemistry, 2009, 8460 and references therein.

For LG=tosylate see for example the literature references Synthetic Communications, 2012, 1715; Synthesis 2015, 2055 or Bioorganic and Medicinal Chemistry Letters, 2013, 2663 and references therein.

For LG=triflate see for example the literature references Bioorganic and Medicinal Chemistry Letters, 2013, 3325 and references therein.

For LG=2,4,6-triisopropylbenzenesulfonate see for example the literature reference WO2010/99379 and references therein.

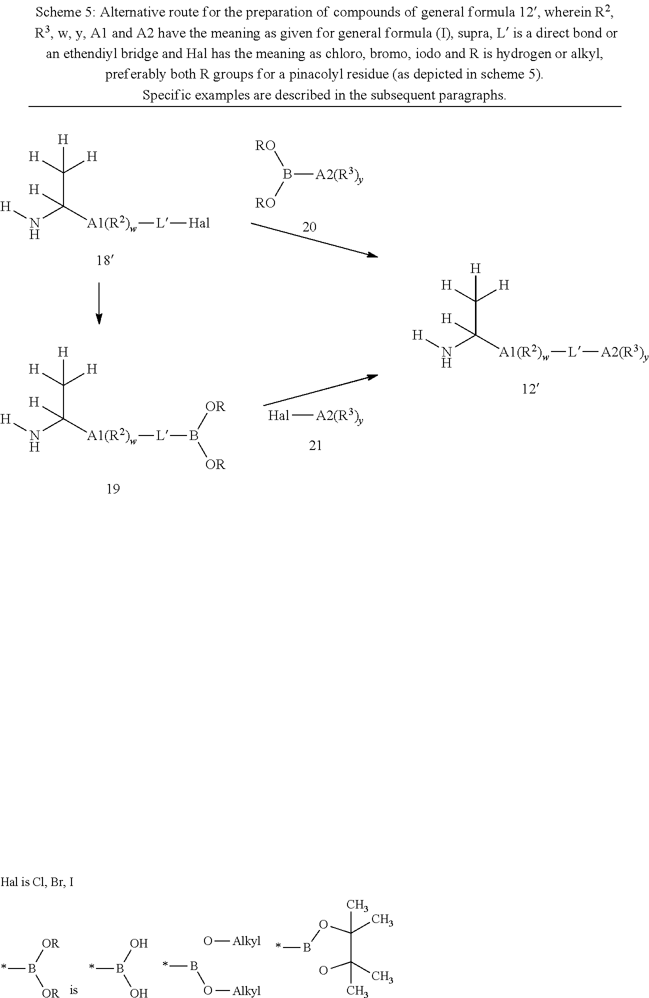

Step 18+20→12 (Scheme 5)

C—C Cross Coupling Reaction

Halogen compounds of general formula 18′ (Scheme 5) can be reacted with a boronic acid derivative 20 to give a compound of formula 12′. The boronic acid derivative may be a boronic acid (R═—H) or an alkyl ester of the boronic acid, e.g. its isopropyl ester (R═—CH(CH3)2), preferably an ester derived from pinacol. The coupling reaction is catalyzed by palladium catalysts, e.g. by Pd(O) catalysts like tetrakis(triphenylphosphine)palladium(0) [Pd(PPh3)4], tris(dibenzylideneacetone)di-palladium(0) 15 [Pd&(dba)3], or by Pd(II) catalysts like dichlorobis(triphenylphosphine)-palladium(II) [Pd(PPh3)3Cl], palladium(II) acetate and triphenylphosphine or by [1,1′-bis(diphenylphosphino)ferrocene]palladium dichloride. The reaction is preferably carried out in a mixture of a solvent like 1,2-dimethoxyethane, dioxane, DMF, DME, THF, or isopropanol with water and in the presence of a base like potassium carbonate, sodium bicarbonate or potassium phosphate. For a review see D. G. Hall, Boronic Acids, 2005 WILEY-VCH Verlag GmbH & Co. KGaA, Weinheim, ISBN 3-527-30991-8 and references cited therein). The reaction is performed at temperatures ranging from room temperature to the boiling point of the solvent. Further on, the reaction can be performed at temperatures above the boiling point under pressure. The reaction is preferably completed after 1 to 36 hours.

Step 18→19 (Scheme 5)

Formation of Boronates/Boronic Acids

Halogen derivative 18′ are converted to boronic acid derivative 22 in analogy to literature procedures (scheme 5).

For Hal=bromo or iodo and *—B(OR)₂=boronic acid pinacol ester the reaction is typically performed with bis(pinacolato)diboron, a palladium catalyst as for example palladium diacetate or dichloro(1,1′-bis(diphenylphosphanyl)ferrocene)palladium(II) dichloromethane adduct a base as for example potassium acetate or triethylamine in an organic solvent as for example DMF, DMSO, acetonitrile. See for example the literature references WO2010/150192, WO2012/158795 or Journal of Medicinal Chemistry, 2006, 5671 and references therein.

For Hal=chloro and *—B(OR)₂=boronic acid pinacole ester see for example the literature references Organic Letters, 2002, 543 or Journal of Organic Chemistry, 2012, 3543 and references therein.

For Hal=bromo or iodo and *—B(OR)₂=boronic acid or boronic acid methyl ester the reaction is typically performed with butyllithium or magnesium/iodine, boronic acid trimethylester in an organic solvent as for example THF, hexane. See for example the literature references Organic and Biomolecular Chemistry, 2012, 6693, Journal of the American Chemical Society, 2009, 17500 or Organic Letters, 2011, 4479 and references therein.

Step 19+21→12 (Scheme 5)

C—C Cross Coupling Reaction

Halogen compounds of general formula 21 (Scheme 5) can be reacted with a boronic acid derivative 19 to give a compound of formula 12′. The boronic acid derivative may be a boronic acid (R═—H) or an alkyl ester of the boronic acid, e.g. its isopropyl ester (R═—CH(CH₃)₂), preferably an ester derived from pinacol. The coupling reaction is catalyzed by palladium catalysts, e.g. by Pd(O) catalysts like tetrakis(triphenylphosphine)palladium(0) [Pd(PPh3)4], tris(dibenzylideneacetone)di-palladium(0) 15 [Pd&(dba)3], or by Pd(II) catalysts like dichlorobis(triphenylphosphine)-palladium(II) [Pd(PPh3)3Cl], palladium(II) acetate and triphenylphosphine or by [1,1′-bis(diphenylphosphino)ferrocene]palladium dichloride. The reaction is preferably carried out in a mixture of a solvent like 1,2-dimethoxyethane, dioxane, DMF, DME, THF, or isopropanol with water and in the presence of a base like potassium carbonate, sodium bicarbonate or potassium phosphate. For a review see D. G. Hall, Boronic Acids, 2005 WILEY-VCH Verlag GmbH & Co. KGaA, Weinheim, ISBN 3-527-30991-8 and references cited therein). The reaction is performed at temperatures ranging from room temperature to the boiling point of the solvent. Further on, the reaction can be performed at temperatures above the boiling point under pressure. The reaction is preferably completed after 1 to 36 hours.

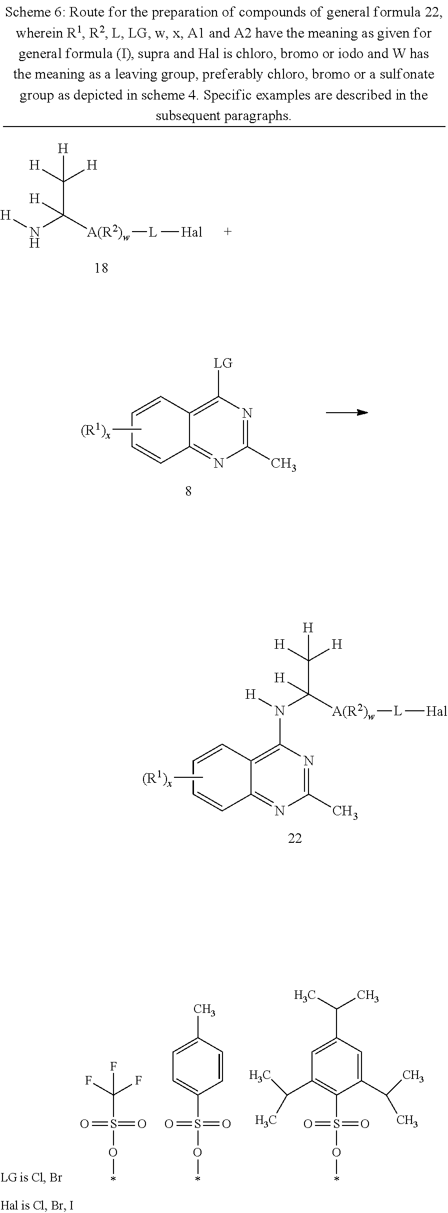

Step 18+8→22 (Scheme 6)

In the first step (scheme 6) amine derivative 18 and quinazoline derivative 8 are converted to amine 22 in analogy to literature procedures. Typically the reaction is performed in an organic solvent such as for example THF, DMF, acetonitrile dichloromethane or isopropyl alcohol with or without a base such as for example triethylamine, N-ethyl-N,N-diisopropylamine, potassium carbonate or potassium tert-butylate.

For LG=chloro see for example the literature references WO2008/86462; WO2008/86462 or European Journal of Medicinal Chemistry, 2015, 462 and references therein.

For LG=bromo see for example the literature references US2009/247519 or Journal of Organic Chemistry, 2009, 8460 and references therein.

For LG=tosylate see for example the literature references Synthetic Communications, 2012, 1715; Synthesis 2015, 2055 or Bioorganic and Medicinal Chemistry Letters, 2013, 2663 and references therein.

For LG=triflate see for example the literature references Bioorganic and Medicinal Chemistry Letters, 2013, 3325 and references therein.

For LG=2,4,6-triisopropylbenzenesulfonate see for example the literature reference WO2010/99379 and references therein.

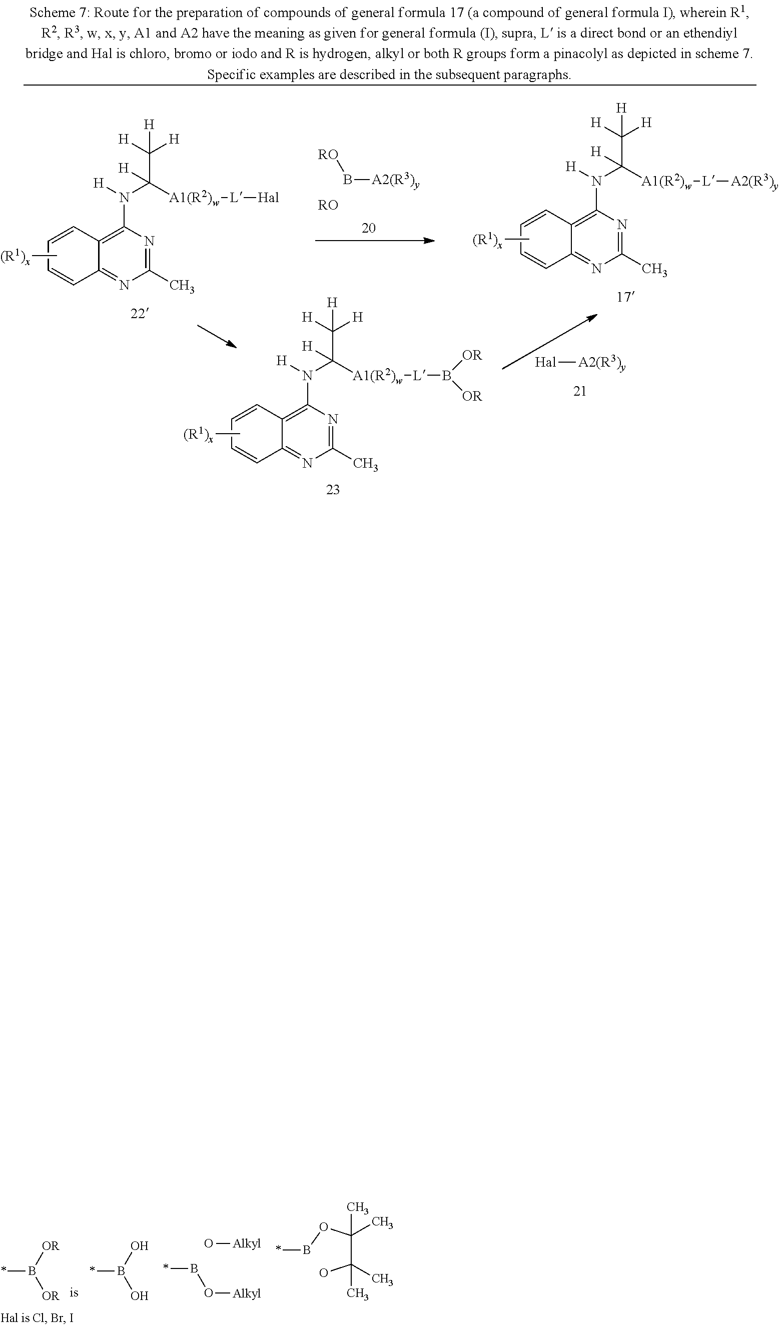

Step 22+20→17 (Scheme 7)

C—C Cross Coupling Reaction

Halogen compounds of general formula 22 (Scheme 7) can be reacted with a boronic acid derivative 20 to give a compound of formula 17. The boronic acid derivative may be a boronic acid (R═—H) or an alkyl ester of the boronic acid, e.g. its isopropyl ester (R═—CH(CH₃)₂), preferably an ester derived from pinacol. The coupling reaction is catalyzed by palladium catalysts, e.g. by Pd(O) catalysts like tetrakis(triphenylphosphine)palladium(0) [Pd(PPh3)4], tris(dibenzylideneacetone)di-palladium(0) [Pd2 (dba)3], or by Pd(II) catalysts like dichlorobis(triphenylphosphine)-palladium(II) [Pd(PPh3)3Cl], palladium(II) acetate and triphenylphosphine or by [1,1′-bis(diphenylphosphino)ferrocene]palladium dichloride. The reaction is preferably carried out in a mixture of a solvent like 1,2-dimethoxyethane, dioxane, DMF, DME, THF, or isopropanol with water and in the presence of a base like potassium carbonate, sodium bicarbonate or potassium phosphate. For a review see D. G. Hall, Boronic Acids, 2005 WILEY-VCH Verlag GmbH & Co. KGaA, Weinheim, ISBN 3-527-30991-8 and references cited therein). The reaction is performed at temperatures ranging from room temperature to the boiling point of the solvent. Further on, the reaction can be performed at temperatures above the boiling point under pressure. The reaction is preferably completed after 1 to 36 hours.

Step 22→23 (Scheme 7)

Formation of Boronates/Boronic Acids

Halogen derivative 22 are converted to boronic acid derivative 23 in analogy to literature procedures (scheme 7).

For Hal=bromo or iodo and *—B(OR)₂=boronic acid pinacole ester the reaction is typically performed with bis(pinacolato)diboron, a palladium catalyst as for example palladium diacetate or dichloro(1,1′-bis(diphenylphosphanyl)ferrocene)palladium(II) dichloromethane adduct a base as for example potassium acetate or triethylamine in an organic solvent as for example DMF, DMSO, acetonitrile. See for example the literature references WO2010/150192, WO2012/158795 or Journal of Medicinal Chemistry, 2006, 5671 and references therein.

For Hal=bromo or iodo and *—B(OR)₂=boronic acid or boronic acid methyl ester the reaction is typically performed with butyllithium or magnesium/iodine, boronic acid trimethylester in an organic solvent as for example THF, hexane. See for example the literature references Organic and Biomolecular Chemistry, 2012, 6693, Journal of the American Chemical Society, 2009, 17500 or Organic Letters, 2011, 4479 and references therein.

### Step 23+21→17 (Scheme 7)

C—C Cross Coupling Reaction

Halogen compounds of general formula 21 (Scheme 7) can be reacted with a boronic acid derivative 23 to give a compound of formula 17. The boronic acid derivative may be a boronic acid (R═—H) or an alkyl ester of the boronic acid, e.g. its isopropyl ester (R═—CH(CH3)2), preferably an ester derived from pinacol. The coupling reaction is catalyzed by palladium catalysts, e.g. by Pd(O) catalysts like tetrakis(triphenylphosphine)palladium(0) [Pd(PPh3)4], tris(dibenzylideneacetone)di-palladium(0) 15 [Pd&(dba)3], or by Pd(II) catalysts like dichlorobis(triphenylphosphine)-palladium(II) [Pd(PPh3)3Cl], palladium(II) acetate and triphenylphosphine or by [1,1′-bis(diphenylphosphino)ferrocene]palladium dichloride. The reaction is preferably carried out in a mixture of a solvent like 1,2-dimethoxyethane, dioxane, DMF, DME, THF, or isopropanol with water and in the presence of a base like potassium carbonate, sodium bicarbonate or potassium phosphate. For a review see D. G. Hall, Boronic Acids, 2005 WILEY-VCH Verlag GmbH & Co. KGaA, Weinheim, ISBN 3-527-30991-8 and references cited therein). The reaction is performed at temperatures ranging from room temperature to the boiling point of the solvent. Further on, the reaction can be performed at temperatures above the boiling point under pressure. The reaction is preferably completed after 1 to 36 hours.

In accordance with a further aspect, the present invention covers intermediate compounds which are useful in the preparation of compounds of the present invention of general formula (I), particularly in the methods described herein. In particular, the present invention covers compounds of general formula II,

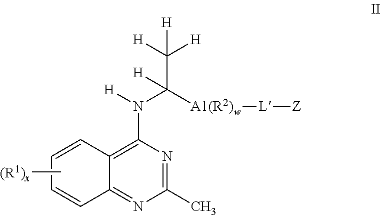

in which R¹, R², A1, R², L′, w and x have the same meanings as defined for the compound of general (I) supra, and Z is

Hal (iodo, bromo or chloro) or

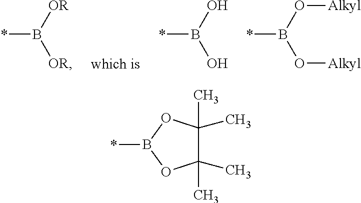

The present invention covers the intermediate compounds which are disclosed in the Example Section of this text, infra.

The present invention covers any sub-combination within any embodiment or aspect of the present invention of intermediate compounds of general formula (II), supra.

In accordance with another aspect, the present invention covers methods of preparing compounds of the present invention, said methods comprising the step as described below and/or the Experimental Section.

In particular, the present invention covers a method to prepare compounds of general formula I supra,

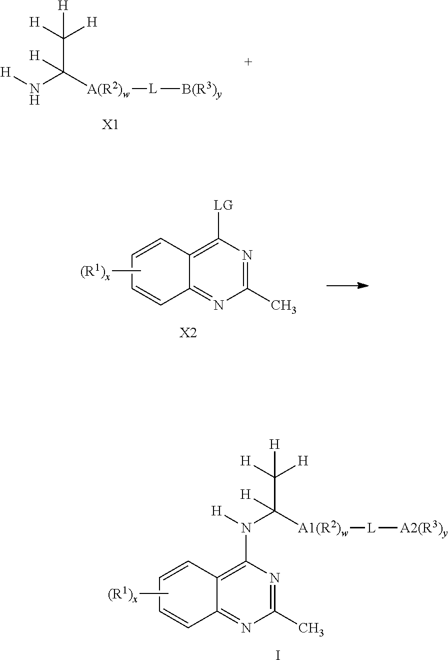

characterized in that compounds of general formula X1 and X2, in which R¹, R², R³, L, w, x, y, A1 and A2 have the same meaning as defined for compounds of general formula (I) and LG is a leaving group as chloro, bromo, iodo, fluoro, triflate, tosylate, mesitylate or nonaflate, are reacted in an organic solvent at a temperature between −20° C. and the boiling point of a solvent, preferably between ambient temperature and the boiling point of the solvent, with or without a base to obtain compounds of general formula I.

The preparation of compounds of general formula I can be performed in a protic or aprotic solvent, preferably in dioxan, tetrahydrofuran, N,N-dimethylformamide, dimethylsulfoxid, methanol, ethanol or 2-propanol.

Preferred bases which can be used for the preparation of compounds of the general formula I are N,N-diisopropylethylamin or triethylamin.

Said compound of general formula I can then optionally be converted into solvates, salts and/or solvates of such salts using the corresponding (i) solvents and/or (ii) bases or acids.

The present invention covers methods of preparing compounds of the present invention of general formula (I), said methods comprising the steps as described in the Experimental Section herein.

The compounds of general formula (I) of the present invention can be converted to any salt, preferably pharmaceutically acceptable salts, as described herein, by any method which is known to the person skilled in the art. Similarly, any salt of a compound of general formula (I) of the present invention can be converted into the free compound, by any method which is known to the person skilled in the art.

For the synthesis of deuterated intermediates and test compounds of the general formula I the same general procedures as described before can be applied by using the corresponding deuterated reagents. For example, compound 3 can be converted to compound 7D by using deuterated acetyl chloride.

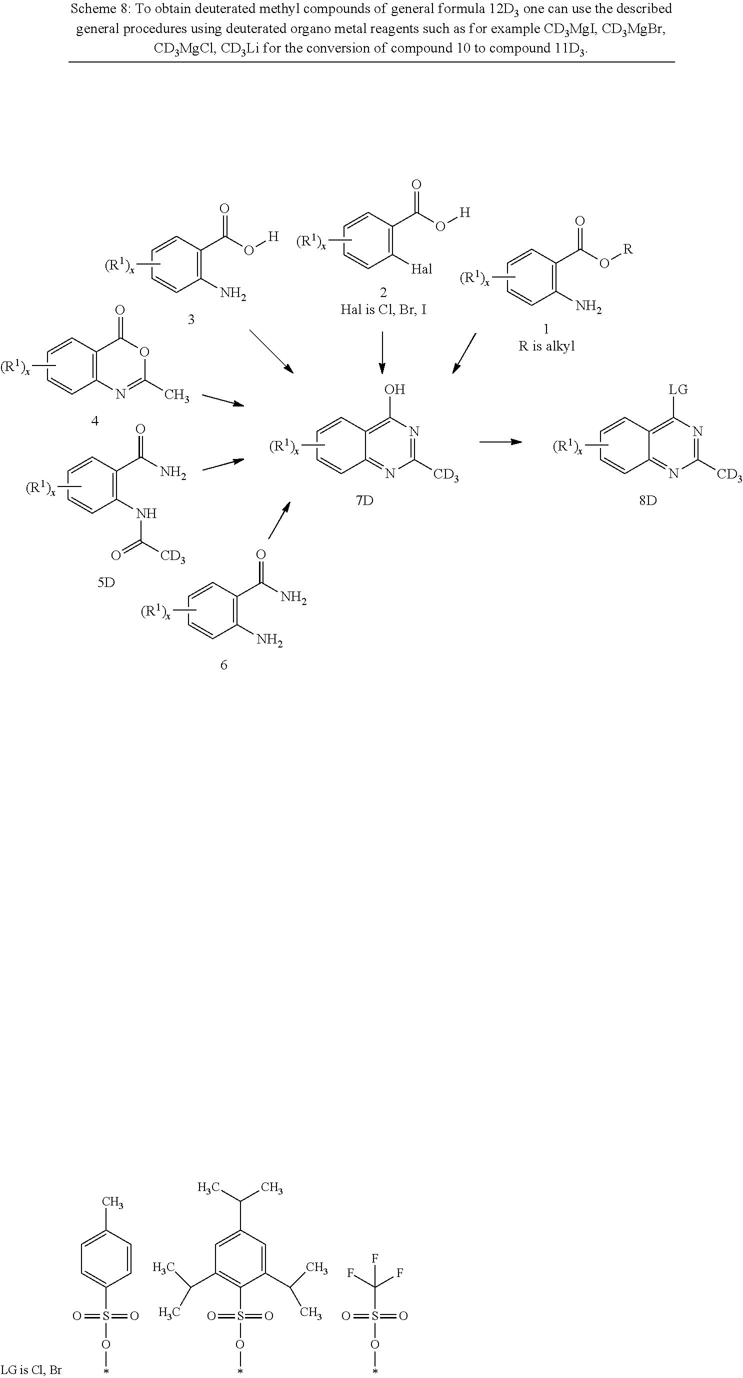

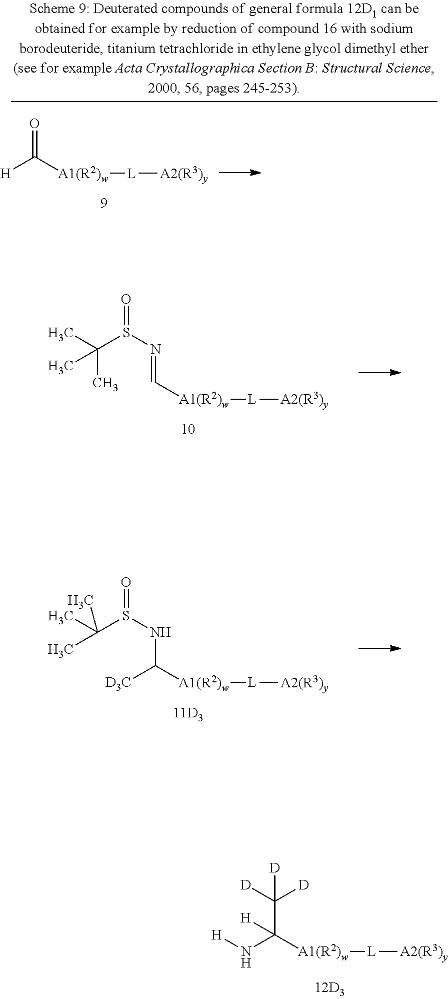

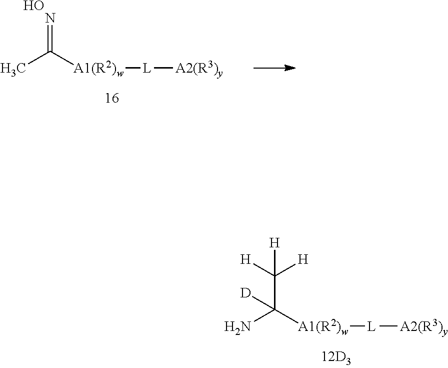

One of the most fundamental characteristics of cancer cells is their ability to sustain chronic proliferation whereas in normal tissues the entry into and progression through the cell division cycle is tightly controlled to ensure a homeostasis of cell number and maintenance of normal tissue function. Loss of proliferation control is emphasized as one of the six hallmarks of cancer [Hanahan D and Weinberg 15 RA, Cell 100, 57, 2000; Hanahan D and Weinberg R A, Cell 144, 646, 2011].

Compounds of general formula (I) of the present invention demonstrate a valuable pharmacological spectrum of action which could not have been predicted. Compounds of the present invention have surprisingly been found to effectively inhibit the Ras-Sos interaction and it is possible therefore that said compounds be used for the treatment or prophylaxis of diseases, preferably hyperproliferative disorders in humans and animals.

Compounds of the present invention can be utilized to inhibit, block, reduce, decrease, etc., cell proliferation and/or cell division, and/or produce apoptosis. This method comprises administering to a mammal in need thereof, including a human, an amount of a compound of general formula (I) of the present invention, or a pharmaceutically acceptable salt, isomer, polymorph, metabolite, hydrate, solvate or ester thereof, which is effective to treat the disorder.

Hyperproliferative disorders include, but are not limited to, for example: psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypophtalmic glioma, cerebellar and cerebral astrocytoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Tumours of the digestive tract include, but are not limited to, anal, colon, colorectal, oesophageal, gallbladder, gastric, pancreatic, rectal, small-intestine, and salivary gland cancers.

Tumours of the urinary tract include, but are not limited to, bladder, penile, kidney, renal pelvis, ureter, urethral and human papillary renal cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, laryngeal, hypopharyngeal, nasopharyngeal, oropharyngeal cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

The present invention also provides methods of treating angiogenic disorders including diseases associated with excessive and/or abnormal angiogenesis.

Inappropriate and ectopic expression of angiogenesis can be deleterious to an organism. A number of pathological conditions are associated with the growth of extraneous blood vessels. These include, for example, diabetic retinopathy, ischemic retinal-vein occlusion, and retinopathy of prematurity [Aiello et al., New Engl. J. Med., 1994, 331, 1480; Peer et al., Lab. Invest., 1995, 72, 638], age-related macular degeneration (AMD) [Lopez et al., Invest. Opththalmol. Vis. Sci., 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumour enlargement and metastasis. Moreover, the growth of new blood and lymph vessels in a tumour provides an escape route for renegade cells, encouraging metastasis and the consequence spread of the cancer. Thus, compounds of general formula (I) of the present invention can be utilized to treat and/or prevent any of the aforementioned angiogenesis disorders, for example by inhibiting and/or reducing blood vessel formation; by inhibiting, blocking, reducing, decreasing, etc. endothelial cell proliferation, or other types involved in angiogenesis, as well as causing cell death or apoptosis of such cell types.

These disorders have been well characterized in humans, but also exist with a similar etiology in other mammals, and can be treated by administering pharmaceutical compositions of the present invention.

The term “treating” or “treatment” as stated throughout this document is used conventionally, for example the management or care of a subject for the purpose of combating, alleviating, reducing, relieving, improving the condition of a disease or disorder, such as a carcinoma.

The compounds of the present invention can be used in particular in therapy and prevention, i.e. prophylaxis, of tumour growth and metastases, especially in solid tumours of all indications and stages with or without pre-treatment of the tumour growth.

Generally, the use of chemotherapeutic agents and/or anti-cancer agents in combination with a compound or pharmaceutical composition of the present invention will serve to:
1. yield better efficacy in reducing the growth of a tumour or even eliminate the tumour as compared to administration of either agent alone,
2. provide for the administration of lesser amounts of the administered chemotherapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.

In addition, the compounds of general formula (I) of the present invention can also be used in combination with radiotherapy and/or surgical intervention.

In a further embodiment of the present invention, the compounds of general formula (I) of the present invention may be used to sensitize a cell to radiation, i.e. treatment of a cell with a compound of the present invention prior to radiation treatment of the cell renders the cell more susceptible to DNA damage and cell death than the cell would be in the absence of any treatment with a compound of the present invention. In one aspect, the cell is treated with at least one compound of general formula (I) of the present invention.

Thus, the present invention also provides a method of killing a cell, wherein a cell is administered one or more compounds of the present invention in combination with conventional radiation therapy.

The present invention also provides a method of rendering a cell more susceptible to cell death, wherein the cell is treated with one or more compounds of general formula (I) of the present invention prior to the treatment of the cell to cause or induce cell death. In one aspect, after the cell is treated with one or more compounds of general formula (I) of the present invention, the cell is treated with at least one compound, or at least one method, or a combination thereof, in order to cause DNA damage for the purpose of inhibiting the function of the normal cell or killing the cell.

In other embodiments of the present invention, a cell is killed by treating the cell with at least one DNA damaging agent, i.e. after treating a cell with one or more compounds of general formula (I) of the present invention to sensitize the cell to cell death, the cell is treated with at least one DNA damaging agent to kill the cell. DNA damaging agents useful in the present invention include, but are not limited to, chemotherapeutic agents (e.g. cis platin), ionizing radiation (X-rays, ultraviolet radiation), carcinogenic agents, and mutagenic agents.

In other embodiments, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of general formula (I) of the present invention is administered to a cell prior to the radiation or other induction of DNA damage in the cell. In another aspect of the invention, a compound of general formula (I) of the present invention is administered to a cell concomitantly with the radiation or other induction of DNA damage in the cell. In yet another aspect of the invention, a compound of general formula (I) of the present invention is administered to a cell immediately after radiation or other induction of DNA damage in the cell has begun.

In another aspect, the cell is in vitro. In another embodiment, the cell is in vivo.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal, intralumbal or intratumoral) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropyl-methylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

In accordance with another aspect, the present invention covers pharmaceutical combinations, in particular medicaments, comprising at least one compound of general formula (I) of the present invention and at least one or more further active ingredients, in particular for the treatment and/or prophylaxis of a hyper-proliferative disorder, in particular cancer.

Particularly, the present invention covers a pharmaceutical combination, which comprises:
one or more first active ingredients, in particular compounds of general formula (I) as defined supra, and
one or more further active ingredients, in particular those used for treatment of hyper-proliferative disorder, in particular cancer.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The compounds of the present invention can be administered as the sole pharmaceutical agent or in combination with one or more other pharmaceutically active ingredients where the combination causes no unacceptable adverse effects. The present invention also covers such pharmaceutical combinations. For example, the compounds of the present invention can be combined with known anti-tumor agents (cancer therapeutics).

Examples of anti-tumor agents (cancer therapeutics) include: 131I-chTNT, abarelix, abiraterone, aclarubicin, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, aprepitant, arcitumomab, arglabin, arsenic trioxide, asparaginase, axitinib, azacitidine, basiliximab, belotecan, bendamustine, besilesomab, belinostat, bevacizumab, bexarotene, bicalutamide, bisantrene, bleomycin, blinatumomab, bortezomib, buserelin, bosutinib, brentuximab vedotin, busulfan, cabazitaxel, cabozantinib, calcitonine, calcium folinate, calcium levofolinate, capecitabine, capromab, carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, eculizumab, edrecolomab, elliptinium acetate, elotuzumab, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, necitumumab, nedaplatin, nelarabine, neridronic acid, netupitant/palonosetron, nivolumabpentetreotide, nilotinib, nilutamide, nimorazole, nimotuzumab, nimustine, nintedanib, nitracrine, nivolumab, obinutuzumab, octreotide, ofatumumab, olaparib, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, rolapitant, romidepsin, romiplostim, romurtide, roniciclib, samarium (153Sm) lexidronam, sargramostim, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

Based upon standard laboratory techniques known to evaluate compounds useful for the treatment of hyper-proliferative disorders, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### EXPERIMENTAL SECTION

The following table 1 lists the abbreviations used in this paragraph and in the Examples section as far as they are not explained within the text body. Other abbreviations have their meanings customary per se to the skilled person.

TABLE 1
Abbreviations

| | |
|---|---|
| BuLi | Butyllithium |
| DCE | Dichloroethane |
| DCM | Dichloromethane |
| DMF | Dimethylformamide |
| DMSO | Dimethyl sulfoxide |
| EA | Ethyl acetate |
| HPLC, LC | high performance liquid chromatography |
| h | hour |
| LiHMDS | Lithium bis(trimethylsilyl)amide |
| KHMDS | Potassium bis(trimethylsilyl)amide |
| KOtBu | Potassium tert-butoxide |
| min | minute |
| LDA | Lithiumdiisopropylamid |
| MS | mass spectroscopy |
| NMR | nuclear magnetic resonance |
| NaHMDS | Sodium bis(trimethylsilyl)amide |
| PE | Petrol ether |
| Rac | Racemate |
| R_{f} | Retardiation factor |
| Rₜ | Retention time |
| RT | Room temperature |
| TFA | Trifluoroacetic acid |
| THF | Tetrahydrofuran |
| TLC | thin-layer chromatography |

Chemical names were generated using ACD/Name Batch Version 12.01 or Autonom 2000.

All reagents, for which the synthesis is not described in the experimental part, are either commercially available or synthesized as described in literature references.

The various aspects of the invention described in this application are illustrated by the following examples which are not meant to limit the invention in anyway.

The example testing experiments described herein serve to illustrate the present invention and the invention is not limited to the examples given.

### Experimental Section—General Part

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartidges KP-Sil® or KP-NH® in combination with a Biotage autopurifier system (SP4® or Isolera Four®) and eluents such as gradients of hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

### EXPERIMENTAL SECTION—EXAMPLES

Analytical Methods

LC-MS Method 1:
Column: Ascentis Express C18 2.7 μm, 30×2.1 mm
Fragment. potential: 50 V
Mass range: 80-800 m/z
Solvent: A=H₂O+0.1% vol HCOOH
   B=methanol+0.1% vol HCOOH
Gradient: 0-1 min 5% B, 1-4 min 5-100% B 4-5 min 100% B, 5-6 min 100-5% B, 6-6.5 min 5% B
Flow: 0.8 mL/min
Temperature: 30° C.
Injection: 1.0 μL
Detection: MM-ES+APCI+DAD (254 nm)
System time delay: 0.2 min

LC-MS Method 2: MS instrument type: Micromass Quatro Micro; HPLC instrument type: Agilent 1100 Series; UV DAD; column: Chromolith Flash RP-18E 25-2 mm; mobile phase A: 0.0375% TFA in water, mobile phase B: 0.01875% TFA in acetonitrile; gradient: 0.0 min 100% A→1.0 min 95% A→3.0 min 95% A→3.5 min 5% A→3.51 min 5% A→4.0 min 95% A; flow rate: 0.8 mL/min; column temp: 50° C.; UV detection: 220 nm & 254 nm.

LC-MS Method 3:
System: Waters Acquity UPLC-MS: Binary Solvent Manager, Sample Manager/Organizer, PDA, ELSD
Column: Acquity UPLC BEH C18 1.7 μm, 50×2.1 mm
Solvent: A=H₂O+H₂O+0.1% vol. HCOOC (99%)
   B=acetonitrile
Gradient: 0-1.6 min 1-99% B, 1.6-2 min 99% B
Flow: 0.8 mL/min
Temperature: 60° C.
Injection: 2.0 μL
Detection: DAD scan range 210-400 nm+ELSD

LC-MS Method 4:
System: Shimadzu LC-MS: UFLC 20-AD and LCMS 2020 MS detector
Column: Shim-pack XR-ODS 2.2 μm, 3.0×50 mm
Solvent: A=H₂O+0.05% vol. HCOOC (99%)
   B=acetonitrile+0.05% vol. HCOOC (99%)

LC-MS Method 5:
System: Waters Acquity UPLC-MS: Binary Solvent Manager, Sample Manager/Organizer, PDA, ELSD
Column: Acquity UPLC BEH C18 1.7 μm, 50×2.1 mm
Solvent: A=H₂O+0.2% vol. NH₃ (32%)
   B=acetonitrile
Gradient: 0-1.6 min 1-99% B, 1.6-2 min 99% B
Flow: 0.8 mL/min
Temperature: 60° C.
Injection: 2.0 μL
Detection: DAD scan range 210-400 nm+ELSD

LC-MS Method 6:
System: Instrument HPLC: Waters UPLC Acquity; Instrument MS: Waters ZQ
Column: Acquity UPLC BEH C18 1.7 μm, 50×2.1 mm
Solvent: A=H₂O+0.1% vol. HCOOC (99%)
   B=acetonitrile
Gradient: 0-1.6 min 1-99% B, 1.6-1.8 min 99% B, 1.81-2 min 1% B
Flow: 0.8 mL/min
Temperature: 60° C.
Detection: PDA scan range 210-400 nm

LC-MS Method 7:
System: Agilent 1290 UHPLC-MS Tof
Column: BEH C 18 (Waters) 1.7 μm, 50×2.1 mm
Solvent: A=H₂O+0.05% vol. HCOOC (99%)
   B=acetonitrile+0.05% vol. HCOOC (99%)
Gradient: 0-1.7 min 2-90% B, 1.7-2 min 90% B, 2-2.5 min 90-2% B
Flow: 1.2 mL/min
Temperature: 60° C.
Detection: DAD scan range 210-400 nm

LC-MS Method 8:
System: Waters Acquity UPLC-MS: Binary Solvent Manager, Sample Manager/Organizer, PDA, ELSD
Column: Acquity UPLC BEH C18 1.7 μm, 50×2.1 mm
Solvent: A=H₂O+0.1% vol. HCOOC (99%)
   B=acetonitrile
Gradient: 0-1.6 min 1-99% B, 1.6-2 min 99% B
Flow: 0.8 mL/min
Temperature: 60° C.
Injection: 2.0 μL
Detection: DAD scan range 210-400 nm+ELSD

LC-MS Method 9:
System: Waters Acquity UPLC-MS SingleQuad
Column: Kinetex C 18 (Phenomenex) 2.6 μm, 50×2.1 mm
Solvent: A=H₂O+0.05% vol. HCOOC (99%)
   B=acetonitrile+0.05% vol. HCOOC (99%)
Gradient: 0-0.2 min 2% B, 0.2-1.7 min 2-90% B, 1.7-1.9 min 90% B, 1.9-2 min 90-2% B, 2-2.5 min 2% B
Flow: 1.3 mL/min
Temperature: 60° C.
Detection: DAD scan range 210-400 nm

LC-MS method 10:
System: Waters Acquity UPLC-MS SingleQuad
Column: Acquity UPLC BEH C18 1.7 μm, 50×2.1 mm
Solvent: A=H2O+0.2% vol. NH₃ (32%) B=acetonitrile
Gradient: 0-1.6 min 1-99% B, 1.6-2 min 99% B
Flow: 0.8 mL/min
Temperature: 60° C.
Detection: DAD scan range 210-400 nm

Preparative HPLC

a) Autopurifier: Acidic Conditions

System: Waters Autopurification system: Pump 2545, Sample Manager 2767, CFO, DAD 2996, ELSD 2424, SQD
Column: XBrigde C18 5.0 μm 100×30 mm
Solvent: A=H₂O+0.1% vol. HCOOH (99%)
   B=acetonitrile
Gradient: 0-0.5 min 5% B 25 mL/min, 0.51-5.5 min 10-100% B 70 mL/min, 5.51-6.5 min 100% B 70 mL/min
Temperature: RT
Solution: max. 250 mg/max. 2.5 mL DMSO or DMF
Injection: 1×2.5 mL
Detection: DAD scan range 210-400 nm, MS ESI+, ESI−, scan range 160-1000 m/z
b) Autopurifier: Basic Conditions

System: Waters Autopurification system: Pump 2545, Sample Manager 2767, CFO, DAD 2996, ELSD 2424, SQD
Column: XBrigde C18 5.0 μm 100×30 mm
Solvent: A=H₂O+0.2% vol. NH₃ (32%)
   B=acetonitrile
Gradient: 0-0.5 min 5% B 25 mL/min, 0.51-5.5 min 10-100% B 70 mL/min, 5.51-6.5 min 100% B 70 mL/min
Temperature: RT
Solution: max. 250 mg/max. 2.5 mL DMSO or DMF
Injection: 1×2.5 mL
Detection: DAD scan range 210-400 nm, MS ESI+, ESI−, scan range 160-1000 m/z

Method X1:

Instrument: Labomatic HD5000, Labocord-5000; Gilson GX-241, Labcol Vario 4000; Column: Chiralpak IE 5 μm 250×20 mm; Eluent A: MTBE+0.1% vol. Diethylamine (99%); Eluent B: Ethanol; Isocratic: 90% A+10% B; Flow 30.0 mL/min; UV 254 nm.

Method X2:

Instrument: Labomatic HD5000, Labocord-5000; Gilson GX-241, Labcol Vario 4000; Column: Chiralpak IA 5 μm 250×30 mm; Eluent A: MTBE+0.1% vol. Diethylamine (99%); Eluent B: Ethanol; Isocratic: 85% A+15% B; Flow 40.0 mL/min; UV 254 nm.

Method X3:

Instrument: Labomatic HD5000, Labocord-5000; Gilson GX-241, Labcol Vario 4000, Column: Chiralpak IA 5.0 μm 250×30 mm; Eluent: 100% Acetonitrile; Flow 50.0 mL/min; UV 280 nm.

Method X4:

Instrument: Waters Autopurification system; Column: Waters XBrigde C18 5.0 μm 100×30 mm; Eluent A: H₂O+0.2% vol. NH₃ (32%), Eluent B: Acetonitrile; Gradient: 0.00-0.50 min 8% B (25→70 mL/min), 0.51-5.50 min 8-15% B (70 mL/min), DAD scan: 210-400 nm.

Method X5:

Instrument: Labomatic HD5000, Labocord-5000; Gilson GX-241, Labcol Vario 4000, Column: Chiralpak IF 5.0 μm 250×30 mm; Eluent A: Hexane+0.1% vol. Diethylamine (99%); Eluent B: Ethanol; Isocratic: 90% A+10% B; Flow 50.0 mL/min; UV 280 nm.

Method X6:

Instrument: Waters Autopurification system; Column: Waters XBrigde C18 5.0 μm 100×30 mm; Eluent A: H₂O+0.2% vol. NH₃ (32%), Eluent B: Acetonitrile; Gradient: 0.00-0.50 min 30% B (25≥70 mL/min), 0.51-5.50 min 30-45% B (70 mL/min), DAD scan: 210-400 nm.

Method X7:

Instrument: Labomatic HD5000, Labocord-5000; Gilson GX-241, Labcol Vario 4000, Column: Chiralpak ID 5.0 μm 250×30 mm; Eluent A: Hexane+0.1% vol Diethylamin (99%); Eluent B: 2-Propanol; Isocratic: 85% A+15% B; Flow 50.0 mL/min; UV 254 nm.

### PREPARATION OF INTERMEDIATES

### General Remarks for the Preparation of Ethanamine of General Formula I

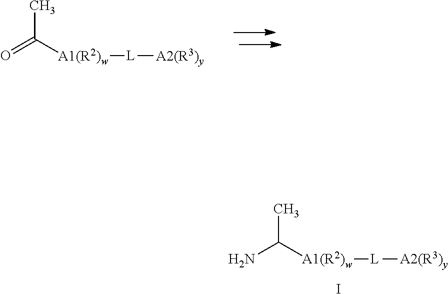

Aromatic or heteroaromatic ethanamines of general structure I have been prepared as described in the literature, for example in Synlett, 2015 (26) 201-204 using commercially available aromatic or heteroaromatic methyl ketones. Not commercially available aromatic or heteroaromatic methyl ketones have been prepared as described in WO 2013/185103.

Representative procedure for preparation of aryl-ethanamines from the corresponding bromides:

### Intermediate INT-1

### 1-(5,6,7,8-tetrahydronaphthalen-1-yl)ethanamine

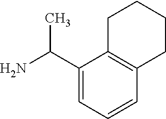

### Step a

### 5-(1-ethoxyvinyl)-1,2,3,4-tetrahydronaphthalene

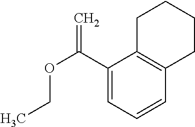

To 5-bromo-1,2,3,4-tetrahydronaphthalene (commercially available; 1.00 g, 4.74 mmol) in 10 mL DMF were added tributyl(1-ethoxyethenyl)stannane (2.1 mL, 6.2 mmol) and tetrakis(triphenylphosphino)palladium(0) (1.09 g, 0.95 mmol). The reaction mixture was heated to 110° C. for 16 h. The reaction mixture was poured into brine. The aqueous layer was extracted with ethyl acetate. The combined organic layers were washed with water and brine, dried over sodium sulfate and evaporated.

The obtained crude material was used without further purification in step b.

### Step b

### 1-(5,6,7,8-tetrahydronaphthalen-1-yl)ethanone

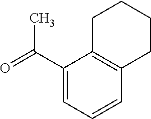

To the crude material from step a in 26 mL THE was added hydrochloric acid (7.1 mL, 2.0 M in water, 14 mmol). The mixture was stirred for 16 h. The reaction mixture was carefully poured into saturated aqueous sodium hydrogen carbonate solution. The aqueous layer was extracted with ethyl acetate. The combined organic layers were washed with brine, dried over sodium sulfate and evaporated. The obtained crude product was purified by flash chromatography to give 1-(5,6,7,8-tetrahydronaphthalen-1-yl)ethanone (560 mg). LCMS (Method 5): Rₜ=1.26 min; MS (ESIpos): m/z=174.9 [M+H]⁺

Step c:

### N-hydroxy-1-(5,6,7,8-tetrahydronaphthalen-1-yl)ethanimine

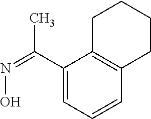

To 1-(5,6,7,8-tetrahydronaphthalen-1-yl)ethanone (560 mg, 3.21 mmol) in 11 mL ethanol were added sodium acetate (2.64 g, 32.1 mmol) and hydroxylamine hydrochloride (1.12 g, 16.1 mmol). The mixture was stirred for 16 h at 40° C. The solvent was evaporated. Ethyl acetate and 6 mL of 2 M hydrochloric acid were carefully added. Brine was added. The aqueous layer was extracted with ethyl acetate. The combined organic layers were dried over sodium sulfate and evaporated. The obtained crude material (780 mg) was used without further purification in step d.

### Step d

### 1-(5,6,7,8-tetrahydronaphthalen-1-yl)ethanamine

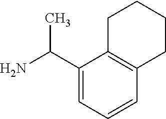

To the crude material from step c (608 mg) in 46 mL methanol were added ammonium chloride (10.3 g, 193 mmol) and zinc powder (10.5 g, 161 mmol). The mixture was stirred at 60° C. for 16 h. The precipitate was filtered off. The solution was concentrated under reduced pressure. The remaining material was suspended in water. Aqueous ammonia solution was added and ethyl acetate was added. The aqueous layer was extracted with ethyl acetate. The combined organic layers were dried over sodium sulfate and evaporated. The obtained crude product was used without further purification in the subsequent step as described in the corresponding product examples.

Representative procedure for the preparation of aryl-ethanamines from the corresponding methyl ketones:

### Intermediate INT-2

### 1-(1-methyl-1H-indazol-4-yl)ethanamine

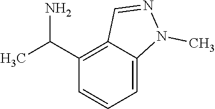

### Step a

### N-hydroxy-1-(1-methyl-1H-indazol-4-yl)ethanimine

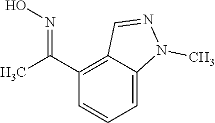

1-(1-methyl-1H-indazol-4-yl)ethanone (956 mg, 5.49 mmol), hydroxylamine hydrochloride (1:1) (1.91 g, 27.4 mmol) and sodium acetate (4.50 g, 54.9 mmol) in 23 mL ethanol were stirred at 40° C. for 19 h. The reaction mixture was diluted with ethyl acetate and water was added. The aqueous layer was extracted with ethyl acetate. The organic layer was dried over sodium sulfate and evaporated. The obtained crude product was used without further purification in step b. LCMS (Method 5): Rₜ=0.83 min; MS (ESIpos): m/z=190.0 [M+H]⁺.

### Step b

### 1-(1-methyl-1H-indazol-4-l )ethanamine

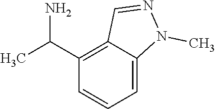

To N-hydroxy-1-(1-methyl-1H-indazol-4-yl)ethanimine (crude material from step a) in 78 mL methanol were added ammonium chloride (17.6 g, 329 mmol) and zinc powder (17.9 g, 274 mmol). The resulting mixture was stirred at 60° C. for 18 h. The remaining solid material was filtered off and washed with methanol. The obtained solution was concentrated. Water was added. The mixture was brought to basic pH by addition of aqueous ammonia (33% in water). The aqueous layer was extracted with ethyl acetate. The organic layer was washed with brine, dried over sodium sulfate and evaporated to give 908 mg of the title compound which was used without further purification. LCMS (Method 5): Rₜ=0.74 min; MS (ESIpos): m/z=175.9 [M+H]⁺. ¹H-NMR (400 MHz, DMSO-d₆): δ [ppm]=8.21 (1H), 7.50-7.40 (1H), 7.36-7.27 (1H), 7.20-7.10 (1H), 4.47-4.36 (1H), 4.02 (3H), 1.36 (3H).

The following ethanamines have been prepared in analogy to the representative procedures described for the intermediates INT-1 and INT-2:

| | | Name of | |
|---|---|---|---|
| Intermediate | Starting material | Intermediate | Structure |
| INT-3 | 6-Acetyl-2H-1,4- benzoxazin-3(4H)- one (commercially available) | 6-[1-aminoethyl]- 2H-1,4-benzo- xazin-3(4H)- one | 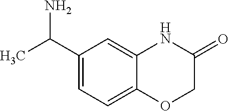 |
| INT-4 | 2-Acetyl-6-meth- oxynaphtalin (commercially available) | 1-(6-methoxy- naphthalen-2- yl)ethanamine | 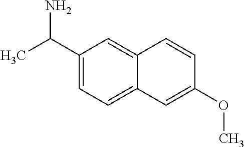 |
| INT-5 | 2-Acetyl-7-meth- oxybenzofuran (commercially available) | 1-(7-methoxy-1- benzofuran-2- yl)ethanamine | 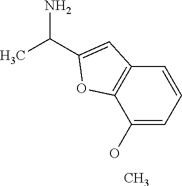 |
| INT-6 | 3-Acetyl-2- methylimidazo- [1,2-a]pyridine (commercially available) | 1-(2-methyl- imidazo[1,2-a]- pyridin-3-yl)- ethanamine | 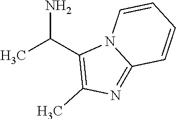 |
| INT-7 | 6-Acetyltetralin (prepared according to J. of Medicinal Chemistry, 1994, vol. 37, #21, 3482) | 1-(5,6,7,8-tetra- hydronaphthalen- 2-yl)ethanamine | 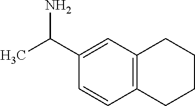 |
| INT-8 | 5-Acetyl-2,3- dihydrobenzo- furan (commer- cially available) | 1-(2,3-dihydro- 1-benzofuran-5- yl)ethanamine | 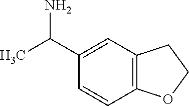 |
| INT-9 | 3′,4′-(Methylene- dioxy)acetophen- one (commer- cially available) | 1-(1,3-benenzo- dioxol-5-yl)ethan- amine | 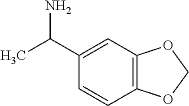 |
| INT-10 | 3-Acetylbenzo- [b]thiophene (commercially available) | 1-(1-benzothio- phen-3-yl)ethan- amine | 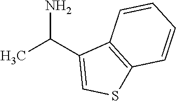 |
| INT-11 | 2-Acetyl-7- hydroxybenzo- furan (commer- cially available) | [1-aminoethyl]- 1-benzofuran-7-ol | 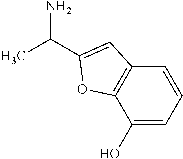 |
| INT-12 | 4-Bromo-2,3- dihydro-1- benzofuran (commercially available) | 1-(2,3-dihydro- 1-benzofuran-4- yl)ethanamine | 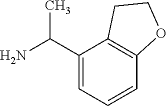 |
| INT-13 | 1-Bromo-2,3- dimethoxy- benzene (commercially available) | 1-(2,3-dimeth- oxyphenyl)ethan- amine | 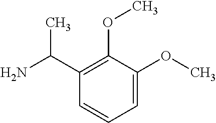 |
| INT-14 | 6-bromo-2,3- dihydro-1,4- benzodioxine (commercially available) | 1-(2,3-dihydro- 1,4-benzodioxin- 6-yl)ethanamine | 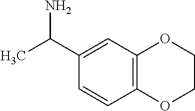 |
| INT-15 | Benzofurane-2- yl-methylketone (prepared according to Journal of Heterocyclic Chemistry, 2015, vol. 52, #5, 1349) | 1-(1-benzofuran- 2-yl)ethanamine | 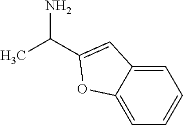 |
| INT-16 | 4-Bromo-6- methyl-1H- indazole (commercially available) | 1-(6-methyl-1H- indazol-4-yl)- ethanamine | 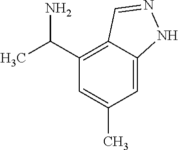 |
| INT-17 | 4-Bromo-5- fluoro-1H- indazole (commercially available) | 1-(5-fluoro-1H- indazol-4-yl)- ethanamine | 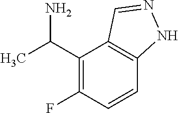 |
| INT-18 | 7-Bromobenzo- thiophene (commercially available) | 1-(1-benzothio- phen-7-yl)ethan- amine | 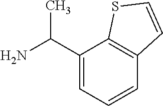 |
| INT-19 | 4-Bromo-1,3- benzothiazole (commercially available) | 1-(1,3-benzo- thiazol-4-yl)- ethanamine | 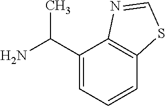 |
| INT-20 | 4-Bromo-3- methyl-1H- indazole (commercially available) | 1-(3-methyl-1H- indazol-4-yl)- ethanamine | 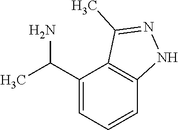 |
| INT-21 | 7-Acetyl-1- methyl-1H- indazole (commercially available) | 1-(1-methyl-1H- indazol-7-yl)- ethanamine | 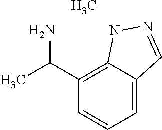 |
| INT-22 | 4-Acetyl-2- methyl-2H- indazole (commercially available) | 1-(2-methyl-2H- indazol-4-yl)- ethanamine | 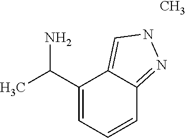 |
| INT-23 | 4-Bromo-5- methyl-1H- indazole (commercially available) | 1-(5-methyl-1H- indazol-4-yl)- ethanamine | 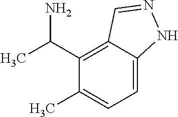 |
| INT-24 | 4-Bromo-7- fluoro-1H- indazole (commercially available) | 1-(7-fluoro-1H- indazol-4-yl)- ethanamine | 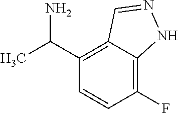 |
| INT-25 | 4-Bromo-6- fluoro-1H- indazole (commercially available) | 1-(6-fluoro-1H- indazol-4-yl)- ethanamine | 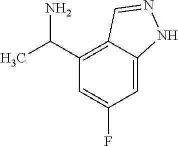 |
| INT-26 | 7-Bromo- benzofuran (commercially available) | 1-(1-benzofuran- 7-yl)ethanamine | 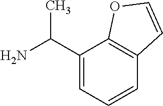 |
| INT-27 | Methyl 4- bromobenzo- [b]thiophene- 2-carboxylate (commercially available) | methyl 4-[1- aminoethyl]-1- benzothiophene- 2-carboxylate | 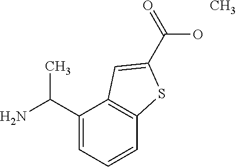 |
| INT-27a | 7-Acetyl-2- methyl-2H- indazole | 1-(2-methyl-2H- indazol-7-yl)- ethanamine | 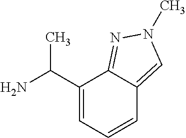 |

### INT-28

### 1-(5-bromothiophen-2-yl)ethanamine

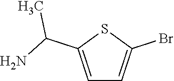

### Step a

### N-[(E)-(5-bromothiophen-2-yl)methylidene]-2-methylpropane-2-sulfinamide

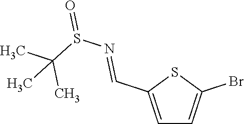

A solution of 5-bromothiophene-2-carbaldehyde (2.5 mL, 21 mmol), 2-methylpropane-2-sulfinamide (3.06 g, 25.2 mmol) and Ti(OEt)₄ (9.1 mL, 42 mmol) in THE (50 mL) was stirred at room temperature overnight. The reaction was quenched with brine (13 mL), diluted with EtOAc and filtered over a celite plug. The crude residue was then dissolved in DCM, filtered over a silica plug and the solvent then removed in vacuo to give the title compound as a yellow solid (6.37 g, quantitative). ¹H-NM R (400 MHz, DMSO-d₆): δ [ppm]=8.24 (s, 1H), 7.42 (d, 1H), 6.92 (d, 1H), 1.16 (s, 9H).

### Step b

### N-[1-(5-bromothiophen-2-yl)ethyl]-2-methylpropane-2-sulfinamide

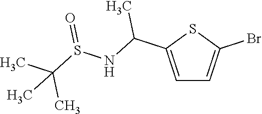

To a solution of N-[(E)-(5-bromothiophen-2-yl)methylidene]-2-methylpropane-2-sulfinamide (2.00 g, 6.80 mmol) in THE (14 mL) was added methylmagnesiumbromide (6.8 mL, 3.0 M, 20 mmol) dropwise and the mixture then stirred at room temperature during 1 hour. The reaction was quenched with NHaCl (sat.), the resulting mixture extracted with DCM and the solvent removed in vacuo to give the title compound as a brown oil (2.03 g, 96%). Main diastereoisomer: ¹H-NM R (400 MHz, DMSO-d₆): δ [ppm]=7.06 (d, 1H), 6.83 (dd, 1H), 5.67 (d, 1H), 4.58 (quin, 1H), 1.55-1.51 (m, 3H), 1.15 (s, 9H).

### Step c

### 1-(5-bromothiophen-2-yl)ethanamine

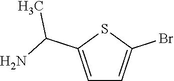

To a solution of N-[1-(5-bromothiophen-2-yl)ethyl]-2-methylpropane-2-sulfinamide (1.05 g, 3.40 mmol) in MeOH (7.0 mL) was added acetyl chloride (730 μL, 10 mmol) dropwise and the mixture then stirred at room temperature during 1 hour. The solvent was removed in vacuo, Et₃N (5.0 mL) and Et₂O (10 mL) added to the residue and the mixture stirred during 1 hour, then the precipitate filtered out and the solvent removed in vacuo to give the title compound as a brown oil (0.47 g, 67%). ¹H-NMR (400 MHz, DMSO-d₆): δ [ppm]=7.01 (d, 1H), 6.74 (dd, 1H), 4.16 (qd, 1H), 1.30 (d, 3H).

The following compounds were synthesized in analogy to intermediate INT-28:

| Intermediate # | Structure | Analtical data |
|---|---|---|
| INT-29 | 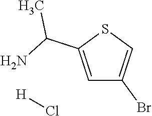 | ¹H-NMR (400 MHz, DMSO-d₆): δ [ppm] = 8.46 (br s, 2H), 7.73 (d, 1H), 7.29 (dd, 1H), 4.71 (q, 1H), 1.56 (d, 3H). |
| INT-30 | 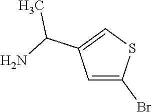 | ¹H-NMR (400 MHz, DMSO-d₆): δ [ppm] = 7.37 (dd, 1H), 7.28 (d, 1H), 4.07 (q, 1H), 1.31 (d, 3H). |
| INT-31 | 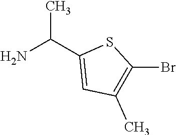 | ¹H-NMR (400 MHz, DMSO-d₆): δ [ppm] = 7.09 (s, 1H), 4.58 (q, 1H), 3.36 (br s, 2H), 2.15-2.05 (m, 3H), 1.54 (br d, 3H). |

### Example 1

### 6,7-dimethoxy-2-methyl-N-[(1R)-1-(naphthalen-1-yl)ethyl]quinazolin-4-amine

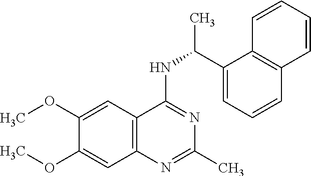 [SMILES: COc1cc2nc(C)nc(N[C@H](C)c3cccc4ccccc34)c2cc1OC]

### Step a

### 2-chloro-6,7-dimethoxy-N-[(1R)-1-(1-naphthyl)ethyl]quinazolin-4-amine

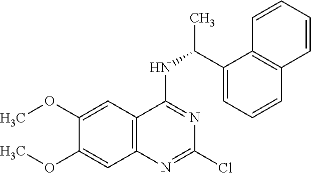 [SMILES: COc1cc2nc(Cl)nc(N[C@H](C)c3cccc4ccccc34)c2cc1OC]

To 2,4-dichloro-6,7-dimethoxyquinazoline (commercially available; 500 mg, 1.93 mmol) in 4 mL THE was added triethylamine (323 μL, 2.32 mmol) and (1R)-1-(1-naphthyl)ethanamine (commercially available; 354 mg, 2.12 mmol). The mixture was stirred at room temperature for d. Saturated aqueous sodium hydrogen carbonate solution was added and the aqueous layer was extracted with ethyl acetate. The combined organic layers were dried over sodium sulfate and evaporated. The resulting solid material was digested with a mixture of dichloromethane and hexane to give 565 mg of the title compound as solid material. LCMS (Method 5): Rₜ=1.34 min; MS (ESIpos): m/z=394.3 [M+H]⁺. ¹H-NMR (500 MHz, DMSO-d₆), δ [ppm]=1.73 (3H), 3.89 (6H), 6.25-6.33 (1H), 7.08 (1H), 7.52 (3H), 7.66-7.71 (1H), 7.80 (1H).

### Step b

### 6,7-dimethoxy-2-methyl-N-[(1R)-1-(naphthalen-1-yl)ethyl]quinazolin-4-amine

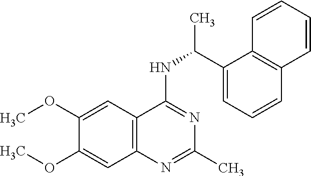 [SMILES: COc1cc2nc(C)nc(N[C@H](C)c3cccc4ccccc34)c2cc1OC]

2-chloro-6,7-dimethoxy-N-[(1R)-1-(1-naphthyl)ethyl]quinazolin-4-amine (60 mg, 0.127 mmol), methyl boronic acid (76 mg, 1.27 mmol) tetrakis(triphenylphoshino)palladium(0) (147 mg, 0.127 mmol) and 2.0 M aqueous sodium carbonate solution (0.76 mL, 1.52 mmol) in 7 mL of 1,4-dioxane were heated to 100° C. for 24 h. The reaction mixture was poured into a mixture of brine and aqueous sodium hydrogen carbonate solution. The aqueous layer was extracted with ethyl acetate, dried over sodium sulfate and evaporated. The obtained crude material was purified by preparative HPLC to give 8 mg of the title compound as solid material. LCMS (Method 5): Rₜ=1.26 min; MS (ESIpos): m/z=374.3 [M+H]⁺. ¹H-NMR (400 MHz, DMSO-d₆), δ [ppm]=1.71 (3H) 2.31-2.35 (3H) 3.87 (6H) 6.44 (1H) 7.03 1H) 7.49-7.57 (3H) 7.61-7.66 (1H) 7.71-7.73 (1H) 7.84 (1H) 7.95 (1H) 8.10 (1H) 8.29 (1H).

### Example 2

### N-[(1R)-1-(3-chlorophenyl)ethyl]-6,7-dimethoxy-2-methylquinazolin-4-amine

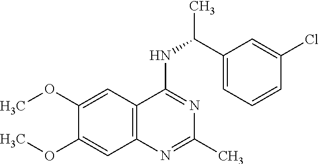 [SMILES: COc1cc2nc(C)nc(N[C@H](C)c3cccc(Cl)c3)c2cc1OC]

The title compound was prepared in analogy to 6,7-dimethoxy-2-methyl-N-[1-(5,6,7,8-tetrahydronaphthalen-1-yl)ethyl]quinazolin-4-amine (example 11) using (1R)-1-(3-chloro-phenyl)ethanamine (commercially available; 286 mg, 1.84 μmol) to give 398 mg of the title compound. LCMS (Method 5): Rₜ=1.24 min; MS (ESIpos): m/z=358.4 [M+H]⁺. ¹H-NMR (400 MHz, DMSO-d₆) δ[ppm]=1.59 (3H) 2.35 (s, 3H) 3.89 (6H) 5.62 (1H) 7.04 (1H) 7.26-7.31 (1H) 7.36 (1H) 7.39-7.44 (1H) 7.50 (1H) 7.70 (1H) 8.01 (1H).

### Example 3

### methyl 4-{1-[(6,7-dimethoxy-2-methylquinazolin-4-yl)amino]ethyl}-1-benzothiophene-2-carboxylate

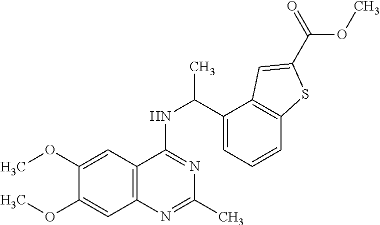

The title compound was prepared in analogy to 6,7-dimethoxy-2-methyl-N-[1-(5,6,7,8-tetrahydronaphthalen-1-yl)ethyl]quinazolin-4-amine (example 11) using methyl 4-[1-aminoethyl]-1-benzothiophene-2-carboxylate (described in INT-27, 96.4 mg, 90% purity, 369 μmol) to give 69 mg of the title compound. LCMS (Method 5): Rₜ=1.25 min; MS (ESIpos): m/z=438.3 [M+H]⁺. ¹H-NMR (400 MHz, DMSO-d₆) δ[ppm]=1.71 (3H), 2.33-2.39 (3H), 3.85 (3H), 3.89 (6H), 6.20 (1H), 7.01 (1H), 7.52-7.58 (1H), 7.60-7.64 (1H), 7.68 (1H), 7.96 (1H), 8.15 (1H), 8.70 (1H).

### Example 4

### N-[1-(1-benzofuran-7-yl)ethyl]-6,7-dimethoxy-2-methylquinazolin-4-amine

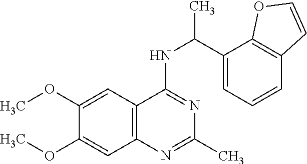 [SMILES: COc1cc2nc(C)nc(NC(C)c3cccc4ccoc34)c2cc1OC]

The title compound was prepared in analogy to 6,7-dimethoxy-2-methyl-N-[1-(5,6,7,8-tetrahydronaphthalen-1-yl)ethyl]quinazolin-4-amine (example 11) using 1-(1-benzofuran-7-yl)ethanamine (described in procedure INT-26, 59.4 mg, 369 μmol) to give 64 mg of the title compound. LCMS (method 5): Rₜ=1.18 min; MS (ESIpos): m/z=364.4 [M+H]⁺. ¹H-NMR (400 MHz, DMSO-d₆) δ[ppm]=1.70 (3H), 2.29 (3H), 3.77-3.97 (6H), 6.14 (1H), 6.98 (1H), 7.03 (1H), 7.18-7.25 (1H), 7.36 (1H), 7.53 (1H), 7.78 (1H), 8.05 (1H), 8.11 (1H).

### Example 5

### N-[1-(7-fluoro-1H-indazol-4-yl)ethyl]-6,7-dimethoxy-2-methylquinazolin-4-amine

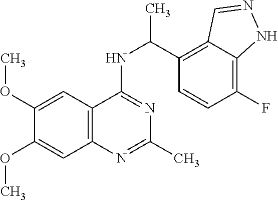 [SMILES: COc1cc2nc(C)nc(NC(C)c3ccc(F)c4[nH]ncc34)c2cc1OC]

Was prepared in analogy to N-[1-(1-benzothiophen-3-yl)ethyl]-6,7-dimethoxy-2-methylquinazolin-4-amine (example 27) using 1-(7-fluoro-1H-indazol-4-yl)ethanamine (described in procedure INT-24; 93.4 mg, 80% purity, 417 μmol) to give 34 mg of the title compound. LCMS (Method 5): Rₜ=0.99 min; MS (ESIpos): m/z=382.4 [M+H]⁺. ¹H-NMR (400 MHz, DMSO-d₆) δ[ppm]=1.70 (3H), 2.31 (3H), 3.83-3.87 (3H), 3.89 (3H), 5.97-6.07 (1H), 7.01 (1H), 7.08-7.18 (2H), 7.71 (1H), 8.06-8.14 (1H), 8.33-8.41 (1H), 13.57-13.66 (1H).

### Example 6

### N-[1-(6-fluoro-1H-indazol-4-yl)ethyl]-6,7-dimethoxy-2-methylquinazolin-4-amine

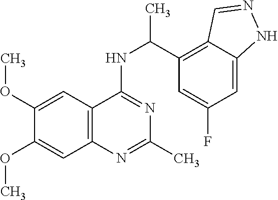 [SMILES: COc1cc2nc(C)nc(NC(C)c3cc(F)cc4[nH]ncc34)c2cc1OC]

The title compound was prepared in analogy to N-[1-(1-benzothiophen-3-yl)ethyl]-6,7-dimethoxy-2-methylquinazolin-4-amine (example 27) using 1-(6-fluoro-1H-indazol-4-yl)ethanamine (described in procedure INT-25; 84.9 mg, 88% purity, 417 μmol) to give 43 mg of the title compound. LCMS (Method 5): Rₜ=0.99 min; MS (ESIpos): m/z=382.4 [M+H]⁺. ¹H-NMR (400 MHz, DMSO-d₆) δ[ppm]=1.70 (3H), 2.31 (3H), 3.86 (3H), 3.91 (3H), 5.97-6.08 (1H), 7.02 (2H), 7.13-7.20 (1H), 7.72 (1H), 8.08-8.15 (1H), 8.29 (1H), 13.08-13.16 (1H).

### Example 7

### 6,7-dimethoxy-2-methyl-N-[1-(5-methyl-1H-indazol-4-yl)ethyl]quinazolin-4-amine

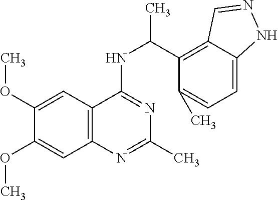 [SMILES: COc1cc2nc(C)nc(NC(C)c3c(C)ccc4[nH]ncc34)c2cc1OC]

The title compound was prepared in analogy to N-[1-(1-benzothiophen-3-yl)ethyl]-6,7-dimethoxy-2-methylquinazolin-4-amine (example 27) using 1-(5-methyl-1H-indazol-4-yl)ethanamine (described in procedure INT-23; 61.7 mg, 85% purity, 299 μmol) to give 53 mg of the title compound. LCMS (Method 5): Rₜ=1.04 min; MS (ESIpos): m/z=378.4 [M+H]⁺. ¹H-NMR (400 MHz, DMSO-d₆) δ[ppm]=1.70 (3H), 2.24 (3H), 2.65 (3H), 3.83 (3H), 3.97 (3H), 5.67-5.77 (1H), 6.96 (1H), 7.11 (1H), 7.20 (1H), 7.85 (1H), 8.21-8.27 (1H), 8.55 (1H), 12.81 (1H).

### Example 8

### 6,7-dimethoxy-2-methyl-N-[1-(2-methyl-2H-indazol-7-yl)ethyl]quinazolin-4-amine

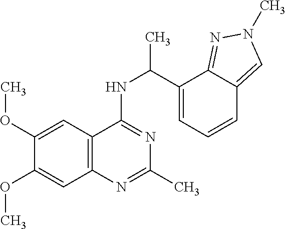 [SMILES: COc1cc2nc(C)nc(NC(C)c3cccc4cn(C)nc34)c2cc1OC]

The title compound was prepared in analogy to N-[1-(1-benzothiophen-3-yl)ethyl]-6,7-dimethoxy-2-methylquinazolin-4-amine (example 27) using 1-(2-methyl-2H-indazol-7-yl)ethanamine (described in procedure INT-27a; 87.5 mg, 84% purity, 0419 μmol) to give 90 mg of the title compound. LCMS (Method 5): Rₜ=1.07 min; MS (ESIpos): m/z=378.4 [M+H]⁺. ¹H-NMR (400 MHz, DMSO-d₆) δ[ppm]=1.71 (3H), 2.29 (3H), 3.87 (3H), 3.94 (3H), 4.23 (3H), 6.12-6.22 (1H), 6.93-7.00 (1H), 7.03 (1H), 7.16-7.21 (1H), 7.52-7.58 (1H), 7.73-7.78 (1H), 8.07 8.14 (1H), 8.36 (1H).

### Example 9

### 6,7-dimethoxy-2-methyl-N-[1-(2-methyl-2H-indazol-4-yl)ethyl]quinazolin-4-amine

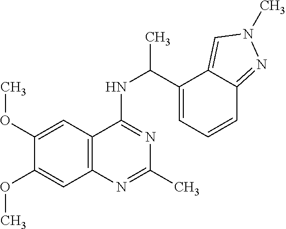 [SMILES: COc1cc2nc(C)nc(NC(C)c3cccc4nn(C)cc34)c2cc1OC]

Was prepared in analogy to N-1-(1-benzothiophen-3-yl)ethyl]-6,7-dimethoxy-2-methylquinazolin-4-amine (example 27) using 1-(2-methyl-2H-indazol-4-yl)ethanamine (described in procedure INT-22; 81.6 mg, 90% purity, 419 μmol) to give 115 mg of the title compound. LCMS (Method 5): Rₜ=0.97 min; MS (ESIpos): m/z=378.5 [M+H]⁺. ¹H-NMR (400 MHz, DMSO-d₆) δ[ppm]=1.68 (3H), 2.34 (3H), 3.84-3.87 (3H), 3.89 (3H), 4.14 (3H), 5.93 6.02 (1H), 7.02 (1H), 7.07 (1H), 7.18 (1H), 7.45 (1H), 7.73 (1H), 8.01-8.06 (1H), 8.45 (1H).

### Example 10

### 6,7-dimethoxy-2-methyl-N-[1-(1-methyl-1H-indazol-7-yl)ethyl]quinazolin-4-amine

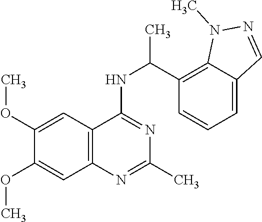 [SMILES: COc1cc2nc(C)nc(NC(C)c3cccc4cnn(C)c34)c2cc1OC]

The title compound was prepared in analogy to N-[1-(1-benzothiophen-3-yl)ethyl]-6,7-dimethoxy-2-methylquinazolin-4-amine (example 27) using 1-(1-methyl-1H-indazol-7-yl)ethanamine (described in procedure INT-21; 74.9 mg, 98% purity, 419 μmol) to give 92 mg of the title compound. LCMS (Method 5): Rₜ=1.07 min; MS (ESIpos): m/z=378.5 [M+H]⁺. ¹H-NMR (400 MHz, DMSO-d₆) δ[ppm]=1.70 (3H), 2.29 (3H), 3.85 (3H), 3.91 (3H), 4.43 (3H), 6.34-6.44 (1H), 7.01 (1H), 7.10 (1H), 7.55 (1H), 7.62 (1H), 7.74 (1H), 8.01 (1H), 8.20-8.27 (1H).

### Example 11

### 6,7-dimethoxy-2-methyl-N-[1-(5,6,7,8-tetrahydronaphthalen-1-yl)ethyl]quinazolin-4-amine

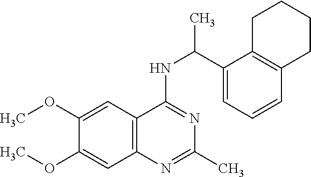 [SMILES: COc1cc2nc(C)nc(NC(C)c3cccc4c3CCCC4)c2cc1OC]

To 4-chloro-6,7-dimethoxy-2-methylquinazoline (commercially available; 106 mg, 445 μmol, commercially available) in 3 mL DMSO was added 1-(5,6,7,8-tetrahydronaphthalen-1-yl)ethanamine (described in procedure INT-1; 85.8 mg, 489 μmol) and N-ethyl-N-isopropylpropan-2-amine (200 μL, 1.2 mmol). The resulting mixture was heated to 130° C. for 2 h in a microwave oven. The reaction mixture was filtered and purified by preparative HPLC to give 63.4 mg of the title compound. LCMS (Method 5): Rₜ=1.37 min; MS (ESIpos): m/z=378.5 [M+H]⁺. ¹H-NMR (400 MHz, DMSO-d₆), δ [ppm]=1.50 (d, 3H), 1.69-1.91 (m, 4H), 2.33 (s, 3H), 2.69-2.81 (m, 3H), 3.22-3.32 (m, 1H), 3.82-3.95 (m, 6H), 5.68 (t, 1H), 6.89 (d, 1H), 6.98-7.07 (m, 2H), 7.32 (d, 1H), 7.72 (s, 1H), 8.05 (d, 1H).

### Example 12

### N-[(1R)-1-(4-fluorophenyl)ethyl]-6,7-dimethoxy-2-methylquinazolin-4-amine

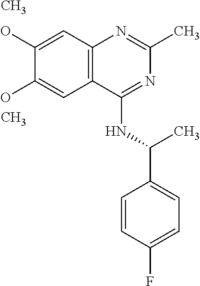

The title compound was prepared in analogy to N-[1-(1-benzothiophen-3-yl)ethyl]-6,7-dimethoxy-2-methylquinazolin-4-amine (example 27) using (1R)-1-(4-fluorophenyl)-ethanamine (commercially available; 170 μL, 1.3 mmol) to give 583 mg of the title compound. LCMS (Method 5): Rₜ=1.15 min; MS (ESIpos): m/z=342.4 [M+H]⁺. ¹H-NMR (400 MHz, DMSO-d₆) δ[ppm]=1.57 (3H), 2.34 (3H), 3.86 (3H), 3.90 (3H), 5.63 (1H), 7.02 (1H), 7.10-7.18 (2H), 7.44-7.51 (2H), 7.68 (1H), 7.98 (1H).

### Example 13

### 6,7-dimethoxy-2-methyl-N-[1-(3-methyl-1H-indazol-4-yl)ethyl]quinazolin-4-amine

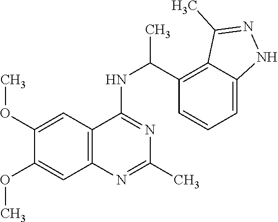 [SMILES: COc1cc2nc(C)nc(NC(C)c3cccc4[nH]nc(C)c34)c2cc1OC]

The title compound was prepared in analogy to N-[1-(1-benzothiophen-3-yl)ethyl]-6,7-dimethoxy-2-methylquinazolin-4-amine (example 27) using 1-(3-methyl-1H-indazol-4-yl)ethanamine (described in procedure INT-20; 92.9 mg, 79% purity, 419 μmol) to give 106 mg of the title compound. LCMS (Method 5): Rₜ=0.97 min; MS (ESIpos): m/z=378.5 [M+H]⁺. ¹H-NMR (400 MHz, DMSO-d₆) δ[ppm]=1.63 (3H), 2.27 (3H), 2.77 (3H), 3.85 (3H), 3.92 (3H), 6.31 (1H), 7.00 (1H), 7.18-7.30 (3H), 7.76 (1H), 8.17 (1H), 12.61 (1H).

### Example 14

### N-[1-(1,3-benzothiazol-4-yl)ethyl]-6,7-dimethoxy-2-methylquinazolin-4-amine

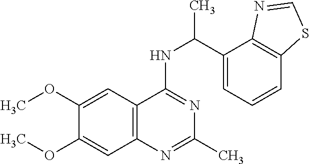 [SMILES: COc1cc2nc(C)nc(NC(C)c3cccc4scnc34)c2cc1OC]

The title compound was prepared in analogy to 6,7-dimethoxy-2-methyl-N-[1-(5,6,7,8-tetrahydronaphthalen-1-yl)ethyl]quinazolin-4-amine (example 11) using 1-(1,3-benzothiazol-4-yl)ethanamine (described in procedure INT-19; 82.2 mg, 461 μmol) to give 39 mg of the title compound. LCMS (Method 5): Rₜ=1.15 min; MS (ESIpos): m/z=381.4 [M+H]⁺. ¹H-NMR (400 MHz, DMSO-d₆) δ[ppm]=1.71 (3H), 2.24 (3H), 3.87 (3H), 3.95 (3H), 6.45 (1H), 7.03 (1H), 7.37-7.48 (1H), 7.57 (1H), 7.81 (1H), 8.04 (1H), 8.19 (1H), 9.47 (1H).

### Example 15

### N-[1-(1-benzothiophen-7-yl)ethyl]-6,7-dimethoxy-2-methylquinazolin-4-amine

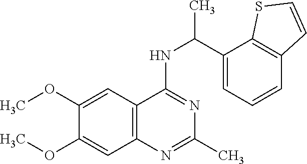 [SMILES: COc1cc2nc(C)nc(NC(C)c3cccc4ccsc34)c2cc1OC]

The title compound was prepared in analogy to N-[1-(1-benzothiophen-3-yl)ethyl]-6,7-dimethoxy-2-methylquinazolin-4-amine (example 27) using 1-(1-benzothiophen-7-yl)ethanamine (described in procedure INT-18; 81.7 mg, 461 μmol) to give 72 mg of the title compound. LCMS (Method 5): Rₜ=1.22 min; MS (ESIpos): m/z=380.4 [M+H]⁺. ¹H-NMR (400 MHz, DMSO-d₆) δ[ppm]=1.71 (d, 3H), 2.30 (s, 3H), 3.88 (d, 6H), 5.89 (t, 1H), 7.03 (s, 1H), 7.35-7.44 (m, 1H), 7.45-7.52 (m, 2H), 7.68-7.82 (m, 3H), 8.18 (d, 1H).

### Example 16

### 6,7-dimethoxy-2-methyl-N-[1-(6-methyl-1H-indazol-4-yl)ethyl]quinazolin-4-amine

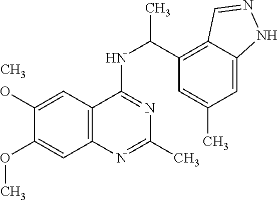

The title compound was prepared in analogy to N-[1-(1-benzothiophen-3-yl)ethyl]-6,7-dimethoxy-2-methylquinazolin-4-amine (example 27) using 1-(6-methyl-1H-indazol-4-yl)ethanamine (described in procedure INT-16; 88.5 mg, 83% purity, 419 μmol) to give 89 mg of the title compound. LCMS (Method 5): Rₜ=1.01 min; MS (ESIpos): m/z=378.5 [M+H]⁺. 1 H-NMR (400 MHz, DMSO-d₆) δ[ppm]=1.68 (3H), 2.33 (3H), 2.41 (3H), 3.82-3.86 (3H), 3.88 (3H), 5.98-6.08 (1H), 6.98-7.02 (2H), 7.16 (1H), 7.71 (1H), 8.05-8.11 (1H), 8.14 (1H), 12.85 12.90 (1H).

### Example 17

### 6,7-dimethoxy-2-methyl-N-[1-(1-methyl-1H-indazol-4-yl)ethyl]quinazolin-4-amine

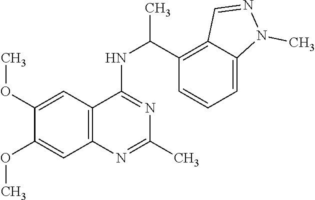 [SMILES: COc1cc2nc(C)nc(NC(C)c3cccc4c3cnn4C)c2cc1OC]

The title compound was prepared in analogy to N-[1-(1-benzothiophen-3-yl)ethyl]-6,7-dimethoxy-2-methylquinazolin-4-amine (example 27) using 1-(1-methyl-1H-indazol-4-yl)ethanamine (described in procedure INT-2; 73.4 mg, 99% purity, 419 μmol) to give 107 mg of the title compound. LCMS (Method 5): Rₜ=1.05 min; MS (ESIpos): m/z=378.5 [M+H]⁺. 1 H-NMR (400 MHz, DMSO-d₆) δ[ppm]=1.70 (3H), 2.30 (3H), 3.85 (3H), 3.90 (3H), 4.01 (3H), 5.99-6.08 (1H), 7.01 (1H), 7.19 (1H), 7.31-7.38 (1H), 7.48 (1H), 7.74 (1H), 8.09-8.14 (1H), 8.25 (1H).

### Example 18

### N-[1-(5-fluoro-1H-indazol-4-yl)ethyl]-6,7-dimethoxy-2-methylquinazolin-4-amine

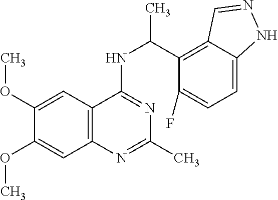 [SMILES: COc1cc2nc(C)nc(NC(C)c3c(F)ccc4[nH]ncc34)c2cc1OC]

The title compound was prepared in analogy to N-[1-(1-benzothiophen-3-yl)ethyl]-6,7-dimethoxy-2-methylquinazolin-4-amine (example 27) using 1-(5-fluoro-1H-indazol-4-yl)ethanamine (described in procedure INT-17; 209 mg, 36% purity, 419 μmol) to give 80 mg of the title compound. LCMS (Method 5): Rₜ=1.01 min; MS (ESIpos): m/z=382.1 [M+H]⁺. ¹H-NMR (400 MHz, DMSO-d₆) δ[ppm]=1.75 (3H), 2.22 (3H), 3.84 (3H), 3.96 (3H), 5.93 (1H), 6.98 (1H), 7.19 (1H), 7.35 (1H), 7.85 (1H), 8.15 (1H), 8.23 (1H), 8.45 (1H), 13.02-13.12 (1H).

### Example 19

### N-[1-(2,3-dihydro-1,4-benzodioxin-6-yl)ethyl]-6,7-dimethoxy-2-methylquinazolin-4-amine

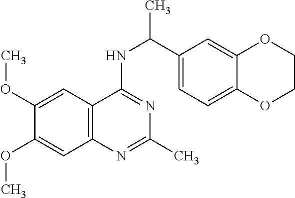 [SMILES: COc1cc2nc(C)nc(NC(C)c3ccc4c(c3)OCCO4)c2cc1OC]

The title compound was prepared in analogy to N-[1-(1-benzothiophen-3-yl)ethyl]-6,7-dimethoxy-2-methylquinazolin-4-amine (example 27) using 1-(2,3-dihydro-1,4-benzodioxin-6-yl)ethanamine (described in procedure INT-14; 84.4 mg, 89% purity, 419 μmol) to give 125 mg of the title compound. LCMS (Method 5): Rₜ=1.12 min; MS (ESIpos): m/z=382.1 [M+H]⁺. ¹H-NMR (400 MHz, DMSO-d₆) δ[ppm]=1.52 (3H), 2.35 (3H), 3.86 (3H), 3.89 (3H), 4.19 (4H), 5.52-5.61 (1H), 6.78 (1H), 6.86-6.91 (1H), 6.93 (1H), 7.01 (1H), 7.66 (1H), 7.85-7.90 (1H).

### Example 20

### N-[1-(1-benzofuran-2-yl)ethyl]-6,7-dimethoxy-2-methylquinazolin-4-amine

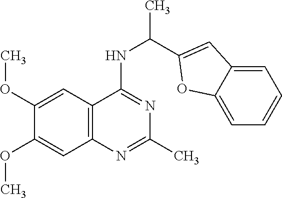 [SMILES: COc1cc2nc(C)nc(NC(C)c3cc4ccccc4o3)c2cc1OC]

The title compound was prepared in analogy to N-[1-(1-benzothiophen-3-yl)ethyl]-6,7-dimethoxy-2-methylquinazolin-4-amine (example 27) using 1-(1-benzofuran-2-yl)ethanamine (described in procedure INT-15; 68.2 mg, 99% purity, 419 μmol) to give 48 mg of the title compound. LCMS (Method 5): Rₜ=1.26 min; MS (ESIpos): m/z=364.1 [M+H]⁺. ¹H-NMR (400 MHz, DMSO-d₆) δ[ppm]=1.68 (3H), 2.41 (3H), 3.87 (6H), 5.88-5.98 (1H), 6.77-6.81 (1H), 7.05 (1H), 7.17-7.28 (2H), 7.52 (1H), 7.58 (1H), 7.67 (1H), 8.12 (1H).

### Example 21

### N-[1-(2,3-dimethoxyphenyl)ethyl]-6,7-dimethoxy-2-methylquinazolin-4-amine

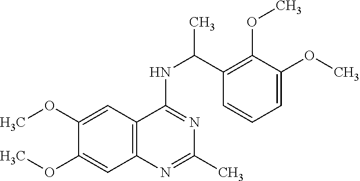

The title compound was prepared in analogy to 6,7-dimethoxy-2-methyl-N-[1-(5,6,7,8-tetrahydronaphthalen-1-yl)ethyl]quinazolin-4-amine (example 11) using 1-(2,3-dimethoxyphenyl)ethanamine (described in procedure INT-13; 92.8 mg, 90% purity, 461 μmol) to give 86 mg of the title compound. LCMS (Method 5): Rₜ=1.12 min; MS (ESIpos): m/z=384.3 [M+H]⁺. ¹H-NMR (400 MHz, DMSO-d₆) δ[ppm] 1.48 (d, 3H), 2.29 (s, 3H), 3.80 (s, 3H), 3.86 (s, 3H), 3.93 (d, 6H), 5.80-5.94 (m, 1H), 6.84-6.92 (m, 1H), 6.95-7.08 (m, 3H), 7.77 (s, 1H), 7.98 (s, 1H).

### Example 22

### N-[1-(2,3-dihydro-1-benzofuran-4-yl)ethyl]-6,7-dimethoxy-2-methylquinazolin-4-amine

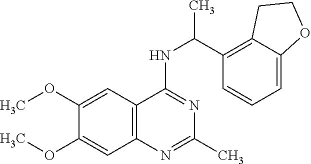 [SMILES: COc1cc2nc(C)nc(NC(C)c3cccc4c3CCO4)c2cc1OC]

The title compound was prepared in analogy to 6,7-dimethoxy-2-methyl-N-[1-(5,6,7,8-tetrahydronaphthalen-1-yl)ethyl]quinazolin-4-amine (example 11) using 1-(2,3-dihydro-1-benzofuran-4-yl)ethanamine (described in procedure INT-12; 83.6 mg, 90% purity, 461 μmol) to give 78 mg of the title compound. LCMS (Method 5): Rₜ=1.12 min; MS (ESIpos): m/z=366.3 [M+H]⁺. ¹H-NMR (400 MHz, DMSO-d₆) δ[ppm] 1.56 (d, 3H), 2.35 (s, 3H), 3.23 (d, 1H), 3.43 (d, 1H), 3.79-3.93 (m, 6H), 4.54 (t, 2H), 5.52 (t, 1H), 6.60 (d, 1H), 6.96 (d, 1H), 6.99 7.11 (m, 2H), 7.70 (s, 1H), 8.01 (d, 1H).

### Example 23

### N-[1-(1,3-benzodioxol-5-yl)ethyl]-6,7-dimethoxy-2-methylquinazolin-4-amine

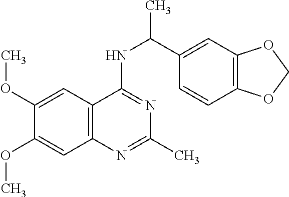 [SMILES: COc1cc2nc(C)nc(NC(C)c3ccc4c(c3)OCO4)c2cc1OC]

The title compound was prepared in analogy to 6-{1-[(6,7-dimethoxy-2-methylquinazolin-4-yl)amino]ethyl}-2H-1,4-benzoxazin-3(4H)-one (example 34) using 1-(1,3-benzodioxol-5-yl)ethanamine (described in procedure INT-9; 112 mg, 74% purity, 503 μmol) to give 110 mg of the title compound. LCMS (Method 5): Rₜ=1.12 min; MS (ESIpos): m/z=368 [M+H]⁺. ¹H-NMR (400 MHz, DMSO-d₆), δ [ppm]=1.54 (3H), 2.36 (3H), 3.83-3.87 (3H), 3.90 (3H), 5.52-5.63 (1H), 5.96 (2H), 6.83 (1H), 6.90 (1H), 6.99-7.04 (2H), 7.67 (1H), 7.85-7.92 (1H).

### Example 24

### N-[1-(2,3-dihydro-1-benzofuran-5-yl)ethyl]-6,7-dimethoxy-2-methylquinazolin-4-amine

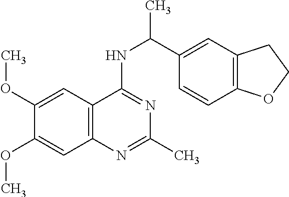 [SMILES: COc1cc2nc(C)nc(NC(C)c3ccc4c(c3)CCO4)c2cc1OC]

The title compound was prepared in analogy to 6-{1-[(6,7-dimethoxy-2-methylquinazolin-4-yl)amino]ethyl}-2H-1,4-benzoxazin-3(4H)-one (example 34) using 1-(2,3-dihydro-1-benzofuran-5-yl)ethanamine (described in procedure INT-8; 132 mg, 62% purity, 503 μmol) to give 123 mg of the title compound. LCMS (Method 5): Rₜ=1.13 min; MS (ESIpos): m/z=366 [M+H]⁺. ¹H-NMR (400 MHz, DMSO-d₆), δ [ppm]=1.54 (3H), 2.36 (3H), 3.10-3.16 (2H), 3.85 (3H), 3.89 (3H), 4.47 (2H), 5.60 (1H), 6.69 (1H), 7.01 (1H), 7.16 (1H), 7.31 (1H), 7.67 (1H), 7.89 (1H).

### Example 25

### 6,7-dimethoxy-2-methyl-N-[1-(5,6,7,8-tetrahydronaphthalen-2-yl)ethyl]quinazolin-4-amine

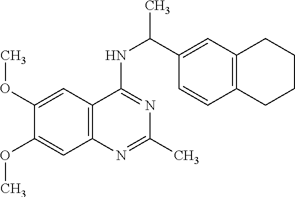 [SMILES: COc1cc2nc(C)nc(NC(C)c3ccc4c(c3)CCCC4)c2cc1OC]

The title compound was prepared in analogy to 6-{1-[(6,7-dimethoxy-2-methylquinazolin-4-yl)amino]ethyl}-2H-1,4-benzoxazin-3(4H)-one (example 34) using 1-(5,6,7,8-tetrahydronaphthalen-2-yl)ethanamine (described in procedure INT-7; 116 mg, 76% purity, 503 μmol) to give 132 mg of the title compound. LCMS (Method 5): Rₜ=0.91 min; MS (ESIpos): m/z=378 [M+H]⁺. ¹H-NMR (400 MHz, DMSO-d₆), δ [ppm]=1.54 (3H), 1.70 (4H), 2.35 (3H), 2.62-2.72 (4H), 3.83-3.87 (3H), 3.89 (3H), 5.58 (1H), 6.94-7.02 (2H), 7.09-7.17 (2H), 7.68 (1H), 7.91 (1H).

### Example 26

### 6,7-dimethoxy-2-methyl-N-[1-(2-methylimidazo[1,2-a]pyridin-3-yl)ethyl]quinazolin-4-amine

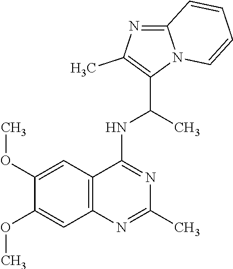 [SMILES: COc1cc2nc(C)nc(NC(C)c3c(C)nc4ccccn34)c2cc1OC]

The title compound was prepared in analogy to 6-{1-[(6,7-dimethoxy-2-methylquinazolin-4-yl)amino]ethyl}-2H-1,4-benzoxazin-3(4H)-one (example 34) using 1-(2-methylimidazo[1,2-a]pyridin-3-yl)ethanamine (described in procedure INT-6; 155 mg, 57% purity, 503 μmol) to give 16.1 mg of the title compound. LCMS (Method 5): Rₜ=0.91 min; MS (ESIpos): m/z=378 [M+H]⁺. ¹H-NMR (400 MHz, DMSO-d₆), δ [ppm]=1.78 (3H), 2.32 (3H), 2.52-2.55 (3H), 3.81-3.86 (3H), 3.89 (3H), 5.85-5.96 (1H), 6.83-6.91 (1H), 7.00 (1H), 7.09-7.16 (1H), 7.38-7.44 (1H), 7.68 (1H), 8.07-8.14 (1H), 8.60-8.66 (1H).

### Example 27

### N-[1-(1-benzothiophen-3-yl)ethyl]-6,7-dimethoxy-2-methylquinazolin-4-amine

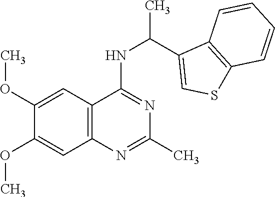 [SMILES: COc1cc2nc(C)nc(NC(C)c3csc4ccccc34)c2cc1OC]

To 4-chloro-6,7-dimethoxy-2-methylquinazoline (commercially available; 100 mg, 419 μmol) in 1.7 mL DMSO was added 1-(1-benzothiophen-3-yl)ethanamine (described in procedure INT-10; 137 mg, 65% purity, 503 μmol) and N-ethyl-N-isopropylpropan-2-amine (190 μL, 1.1 mmol). The resulting mixture was heated to 100° C. for 16 h. The reaction mixture was filtered and purified by preparative HPLC to give 110 mg of the title compound. LCMS (Method 5): Rₜ=1.24 min; MS (ESIpos): m/z=380.4 [M+H]⁺. ¹H-NMR (400 MHz, DMSO-d₆), δ [ppm]=1.71 (3H), 2.44 (3H), 3.79-3.84 (3H), 3.86 (3H), 6.08-6.18 (1H), 7.04 (1H), 7.30-7.37 (2H), 7.62 (1H), 7.72 (1H), 7.87 (1H), 7.94-8.04 (2H).

### Example 28

### 2-{-[(6,7-dimethoxy-2-methy quinazolin-4-yl)amino]ethyl}-1-benzofuran-7-ol

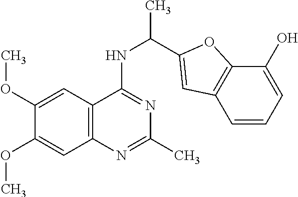 [SMILES: COc1cc2nc(C)nc(NC(C)c3cc4cccc(O)c4o3)c2cc1OC]

The title compound was prepared in analogy to N-[1-(1-benzothiophen-3-yl)ethyl]-6,7-dimethoxy-2-methylquinazolin-4-amine (example 27) using [1-aminoethyl]-1-benzofuran-7-ol (described in procedure INT-11; 109 mg, 82% purity, 503 μmol) to give 33 mg of the title compound. LCMS (Method 5): Rₜ=0.86 min; MS (ESIpos): m/z=380.5 [M+H]⁺. ¹H-NMR (400 MHz, DMSO-d₆), δ [ppm]=1.66 (3H), 2.42 (3H), 3.87 (6H), 5.84-5.96 (1H), 6.65-6.74 (2H), 6.97 (1H), 7.05 (1H), 7.67 (1H), 8.12 (1H), 9.94 (1H).

### Example 29

### 6-bromo-2-methyl-N-[(1R)-1-phenylethyl]quinazolin-4-amine

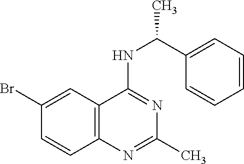 [SMILES: Cc1nc(N[C@H](C)c2ccccc2)c2cc(Br)ccc2n1]

The title compound was prepared in analogy to 6-bromo-N-[(1R)-1-(4-fluorophenyl)ethyl]-2-methylquinazolin-4-amine (example 31) using (1R)-1-phenylethanamine (commercially available; 170 μL, 1.3 mmol) to give 57 mg of the title compound from 90 mg of the title compound. LCMS (Method 5): Rₜ=1.30 min; MS (ESIpos): m/z=343 [M+H]⁺. ¹H-NMR (400 MHz, DMSO-d₆) δ[ppm]=1.58 (d, 3H), 2.39 (s, 3H), 5.62 (t, 1H), 7.17-7.26 (m, 1H), 7.33 (t, 2H), 7.45 (d, 2H), 7.53 (d, 1H), 7.84 (d, 1H), 8.48 (d, 1H), 8.71 (s, 1H).

### Example 30

### 6-{[dimethyl(oxido)-lambda6-sulfanylidene]amino}-2-methyl-N-[(1R)-1-phenylethyl]quinazolin-4-amine

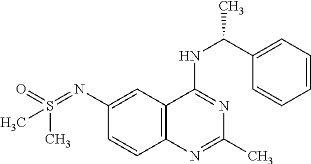 [SMILES: Cc1nc(N[C@H](C)c2ccccc2)c2cc(N=S(C)(C)=O)ccc2n1]

The title compound was prepared in analogy 6-{[dimethyl(oxido)-lambda6-sulfanylidene]amino}-N-[(1R)-1-(4-fluorophenyl)ethyl]-2-methylquinazolin-4-amine (example 32) using 6-bromo-2-methyl-N-[(1R)-1-phenylethyl]quinazolin-4-amine ((described in example 29; 66.4 mg, 0.194 mmol,) to give 9 mg of the title compound. LCMS (Method 5): Rₜ=0.96 min; MS (ESIpos): m/z=355.4 [M+H]⁺. ¹H-NMR (400 MHz, DMSO-d₆), δ [ppm]=1.57 (3H), 2.35 (3H), 3.28 (6H), 5.57-5.71 (1H), 7.21 (1H), 7.31 (2H), 7.38-7.49 (4H), 7.75 (1H), 8.19 (1H).

### Example 31

### 6-bromo-N-[(1R)-1-(4-fluorophenyl)ethyl]-2-methylquinazolin-4-amine

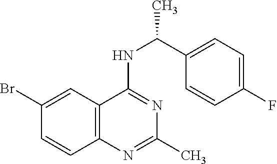 [SMILES: Cc1nc(N[C@H](C)c2ccc(F)cc2)c2cc(Br)ccc2n1]

To 6-bromo-4-chloro-2-methylquinazoline (300 mg, 1.16 mmol, commercially available) in 2 mL DMF was added (1R)-1-(4-fluorophenyl)ethanamine (170 μL, 1.3 mmol, commercially available) and N-ethyl-N-isopropylpropan-2-amine (520 μL, 3.0 mmol). The resulting mixture was heated for 1 h at 100° C. in a microwave oven. The reaction was allowed to cool to room temperature. Brine was added, and the resulting mixture was extracted with ethyl acetate. The organic layer was separated and dried over magnesium sulfate. After evaporation of the solvent, 58 mg of the crude product was purified by preparative HPLC to give 37 mg of the title compound. LCMS (Method 5): Rₜ=1.31 min; MS (ESIpos): m/z=361 [M+H]⁺. ¹H-NMR (400 MHz, DMSO-d₆) δ [ppm]=1.57 (d, 3H), 2.39 (s, 3H), 5.59 (t, 1H), 7.14 (t, 2H), 7.42-7.57 (m, 3H), 7.84 (d, 1H), 8.46 (d, 1H), 8.68 (s, 1H).

### Example 32

### 6-{[dimethyl(oxido)-lambda6-sulfanylidene]amino}-N-[(1R)-1-(4-fluorophenyl)ethyl]-2-methylquinazolin-4-amine

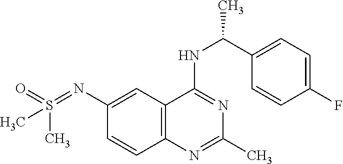 [SMILES: Cc1nc(N[C@H](C)c2ccc(F)cc2)c2cc(N=S(C)(C)=O)ccc2n1]

To the crude material of 6-bromo-N-[(1R)-1-(4-fluorophenyl)ethyl]-2-methylquinazolin-4-amine (220 mg, 0.61 mmol, example 31) in 10 mL DMF were added sodium tert-butoxide (234 mg, 2.44 mmol), (R)-(+)-2,2′-bis(diphenylphosphino)-1-1′-binaphthyl (57 mg, 0.092 mmol), bis(dibenzylideneacetone)palladium (35 mg, 0.061 mmol) and rac-methylsulfonimidoyl)methane (85 mg, 0.92 mmol). The reaction mixture was heated to 100° C. for 3 h in a microwave oven. Brine was added and the aqueous layer was extracted with ethyl acetate, dried over sodium sulfate and evaporated. LCMS (Method 5): Rₜ=0.99 min; MS (ESIpos): m/z=373.4 [M+H]⁺. ¹H-NMR (400 MHz, DMSO-d₆), δ [ppm]=1.56 (3H), 2.35 (3H), 3.27 (6H), 5.61 (1H), 7.13 (2H), 7.36-7.54 (4H), 7.73 (1H), 8.15 (1H).

### Example 33

### 6,7-dimethoxy-N-[1-(7-methoxy-1-benzofuran-2-yl)ethyl]-2-methylquinazolin-4-amine

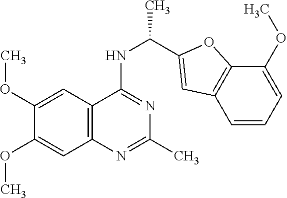 [SMILES: COc1cc2nc(C)nc(N[C@H](C)c3cc4cccc(OC)c4o3)c2cc1OC]

The title compound was prepared in analogy to 6-{1-[(6,7-dimethoxy-2-methylquinazolin-4-yl)amino]ethyl}-2H-1,4-benzoxazin-3(4H)-one (example 34) using 1-(7-methoxy-1-benzofuran-2-yl)ethanamine (described in procedure INT-5; 134 mg, 66% purity, 461 μmol) to give 141.4 mg of the title compound. LCMS (Method 5): Rₜ=1.24 min; MS (ESIpos): m/z=404 [M+H]⁺. ¹H-NMR (400 MHz, DMSO-d₆), δ [ppm]=1.68 (3H), 2.44 (3H), 3.86-3.90 (9H), 5.88 5.97 (1H), 6.79 (1H), 6.88 (1H), 7.05 (1H), 7.10-7.16 (2H), 7.70 (1H), 8.26-8.37 (1H).

### Example 34

### 6-{1-[(6,7-dimethoxy-2-methylquinazolin-4-yl)amino]ethyl}-2H-1,4-benzoxazin-3(4H)-one

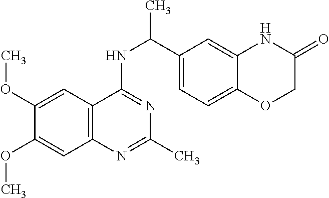 [SMILES: COc1cc2nc(C)nc(NC(C)c3ccc4c(c3)NC(=O)CO4)c2cc1OC]

To 4-chloro-6,7-dimethoxy-2-methylquinazoline (100 mg, 419 μmol, commercially available) in 1.7 mL DMSO was added 6-[1-aminoethyl]-2H-1,4-benzoxazin-3(4H)-one (described in procedure INT-3; 114 mg, 78% purity, 461 μmol) and N-ethyl-N-isopropylpropan-2-amine (190 μL, 1.1 mmol). The resulting mixture was stirred at 100° C. overnight. The reaction mixture was filtered purified by preparative HPLC to give 151 mg of the title compound. LCMS (Method 5): Rₜ=0.92 min; MS (ESIpos): m/z=395 [M+H]⁺. ¹H-NMR (400 MHz, DMSO-d₆), δ [ppm]=1.56 (3H), 2.41 (3H), 3.86-3.89 (3H), 3.90 (3H), 4.52 (2H), 5.55-5.65 (1H), 6.91 (1H), 6.96-7.05 (3H), 7.74 (1H), 8.26-8.36 (1H), 10.68 (1H).

### Example 35

### 6,7-dimethoxy-N-[1-(6-methoxy-2-naphthyl)ethyl]-2-methylquinazolin-4-amine

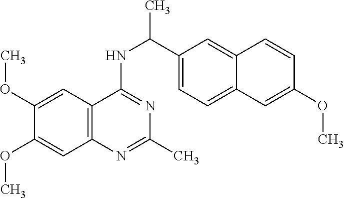 [SMILES: COc1ccc2cc(C(C)Nc3nc(C)nc4cc(OC)c(OC)cc34)ccc2c1]

The title compound was prepared in analogy to 6-{1-[(6,7-dimethoxy-2-methylquinazolin-4-yl)amino]ethyl}-2H-1,4-benzoxazin-3(4H)-one (example 34) using 1-(6-methoxynaphthalen-2-yl)ethanamine (described in procedure INT-4; 116 mg, 80% purity, 461 μmol) to give 151.3 mg of the title compound. LCMS (Method 5): Rₜ=1.24 min; MS (ESIpos): m/z=404 [M+H]⁺. ¹H-NMR (400 MHz, DMSO-d₆), δ [ppm]=1.69 (3H), 2.43 (3H), 3.85 (3H), 3.87-3.90 (3H), 3.92 (3H), 5.80-5.89 (1H), 7.04 (1H), 7.14 (1H), 7.28 (1H), 7.57 (1H), 7.77-7.83 (3H), 7.86 (1H), 8.54-8.67 (1H).

### Example 36

### N-[(1R)-1-(5′-amino-2′-methylbiphenyl-3-yl)ethyl]-6,7-dimethoxy-2-methylquinazolin-4-amine

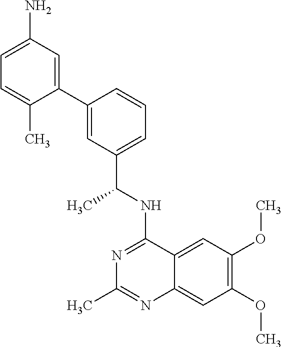 [SMILES: COc1cc2nc(C)nc(N[C@H](C)c3cccc(-c4cc(N)ccc4C)c3)c2cc1OC]

To N-[(1R)-1-(3-bromophenyl)ethyl]-6,7-dimethoxy-2-methylquinazolin-4-amine (prepared as described in example 191; 60.3 mg, 0.15 mmol) in 1 mL NMP were added 4-methyl-3-(4,4,5,5-tetramethyl-1,3,2-dioxaborolan-2-yl)aniline (commercially available; 69.9 mg, 0.3 mmol) in 0.53 mL NM P, [1,1′-bis-(diphenylphosphino)-ferrocene]dichloropalladium(II) dichloromethane complex (25 mg, 0.03 mmol) in 1 mL NMP and potassium carbonate (62.2 mg, 0.45 mmol) in 0.5 mL water. The reaction mixture is heated to 100° C. for 24 h. The reaction mixture is filtered over alumina N. The obtained mixture is purified by preparative HPLC to give 16 mg of the title compound as solid material. LCMS (Method 6): Rₜ=0.78 min; MS (ESIpos): m/z=429.3 [M+H]⁺.

The compounds in the following table were prepared in analogy to example 36. All boronic acids for the Suzuki coupling are commercially available as free boronic acid or its corresponding pinacolate. The preparation of N-[1-(5-bromothiophen-2-yl)ethyl]-6,7-dimethoxy-2-methylquinazolin-4-amine is described in example 209:

| | | | | MW |
|---|---|---|---|---|
| | | | Retention | found |
| | | | time | [M + H]⁺ |
| Example | Structure | Name | [min] | ESI⁺ |
| 37 | 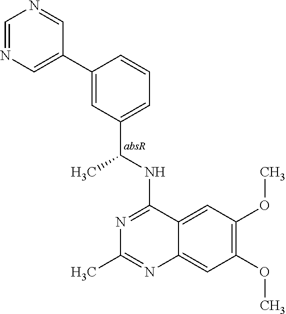 [SMILES: COc1cc2nc(C)nc(N[C@H](C)c3cccc(-c4cncnc4)c3)c2cc1OC] | 6,7-dimethoxy- 2-methyl-N- {(1R)-1-[3- (pyrimidin-5- yl)phenyl]- ethyl}quinazo- lin-4-amine | 0.76 | 402.2 |
| 38 | 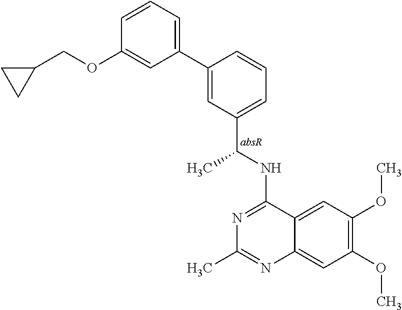 [SMILES: COc1cc2nc(C)nc(N[C@H](C)c3cccc(-c4cccc(OCC5CC5)c4)c3)c2cc1OC] | N-{(1R)-1-[3′- (cyclopropyl- methoxy)bi- phenyl-3-yl]- ethyl}-6,7- dimethoxy-2- methylquinazo- lin-4-amine | 1.07 | 470.2 |
| 39 | 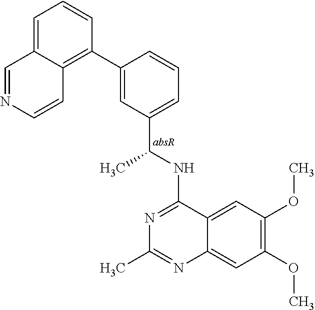 [SMILES: COc1cc2nc(C)nc(N[C@H](C)c3cccc(-c4cccc5cnccc45)c3)c2cc1OC] | N-{(1R)-1-[3- (isoquinolin-5- yl)phenyl]- ethyl}-6,7- dimethoxy-2- methylquinazo- lin-4-amine | 0.76 | 451.2 |
| 40 | 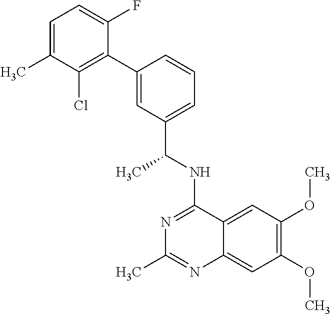 | N-[(1R)-1-(2′- chloro-6′-fluoro- 3′-methylbi- phenyl-3-yl)- ethyl]-6,7- dimethoxy-2- methylquinazo- lin-4-amine | 1.05 | 466.2 |
| 41 | 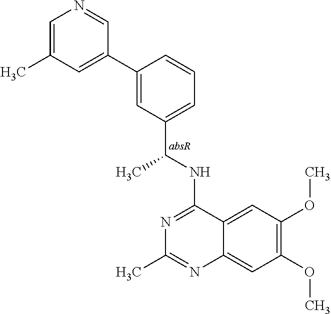 | 6,7-dimethoxy- 2-methyl-N- {(1R)-1-[3-(5- methylpyridin- 3-yl)phenyl]- ethyl}quinazo- lin-4-amine | 0.70 | 415.2 |
| 42 | 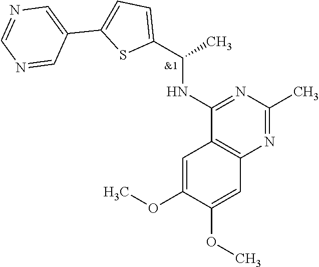 [SMILES: COc1cc2nc(C)nc(N[C@@H](C)c3ccc(-c4cncnc4)s3)c2cc1OC] | 6,7-dimethoxy- 2-methyl-N-{1- [5-(pyrimidin- 5-yl)thiophen-2- yl]ethyl}quinazo- lin-4-amine | 0.74 | 408.1 |
| 43 | 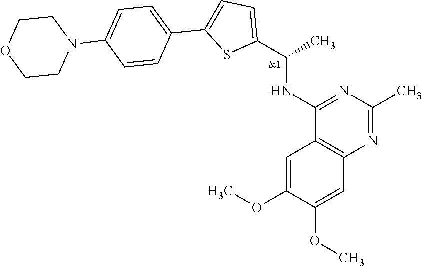 [SMILES: COc1cc2nc(C)nc(N[C@@H](C)c3ccc(-c4ccc(N5CCOCC5)cc4)s3)c2cc1OC] | 6,7-dimethoxy- 2-methyl-N- [1-{5-[4- (morpholin-4- yl)phenyl]thio- phen-2-yl}- ethyl]quinazo- lin-4-amine | 0.94 | 491.2 |
| 44 | 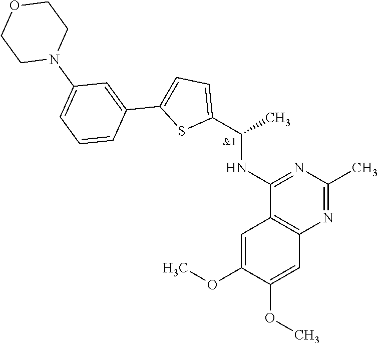 [SMILES: COc1cc2nc(C)nc(N[C@@H](C)c3ccc(-c4cccc(N5CCOCC5)c4)s3)c2cc1OC] | 6,7-dimethoxy- 2-methyl-N- [1-{5-[3- (morpholin-4- yl)phenyl]thio- phen-2-yl}- ethyl]quinazo- lin-4-amine | 0.95 | 491.2 |
| 45 | 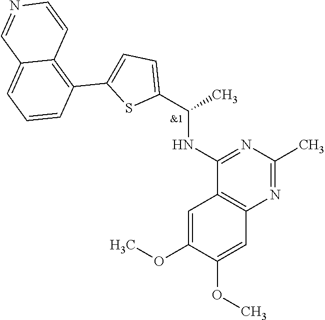 [SMILES: COc1cc2nc(C)nc(N[C@@H](C)c3ccc(-c4cccc5cnccc45)s3)c2cc1OC] | N-{1-[5-(iso- quinolin-5-yl)- thiophen-2-yl]- ethyl}-6,7- dimethoxy-2- methylquinazo- lin-4-amine | 0.77 | 457.2 |
| 46 | 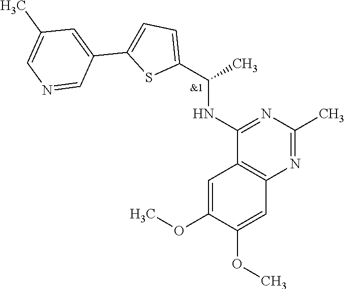 | 6,7-dimethoxy- 2-methyl-N- {1-[5-(5-methyl- pyridin-3-yl)- thiophen-2-yl]- ethyl}quinazo- lin-4-amine | 0.74 | 421.2 |
| 47 | 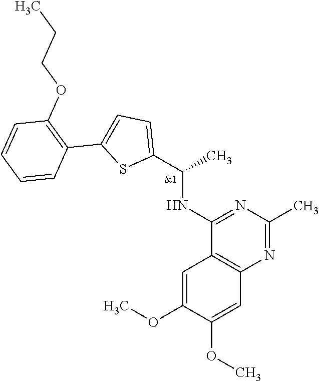 [SMILES: CCCOc1ccccc1-c1ccc([C@H](C)Nc2nc(C)nc3cc(OC)c(OC)cc23)s1] | 6,7-dimethoxy- 2-methyl-N-{1- [5-(2-propoxy- phenyl)thio- phen-2-yl]- ethyl}quinazo- lin-4-amine | 1.06 | 464.2 |
| 48 | 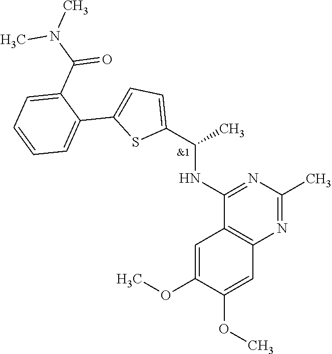 [SMILES: COc1cc2nc(C)nc(N[C@@H](C)c3ccc(-c4ccccc4C(=O)N(C)C)s3)c2cc1OC] | 2-(5-{1-[(6,7- dimethoxy-2- methylquinazo- lin-4-yl)- amino]ethyl}- thiophen-2-yl)- N,N-dimethyl- benzamide | 0.83 | 477.2 |
| 49 | 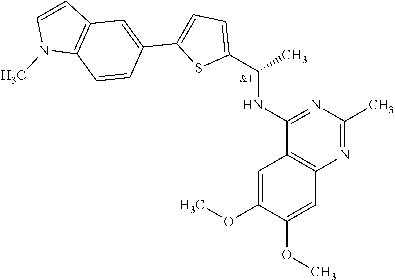 | 6,7-dimethoxy- 2-methyl-N- {1-[5-{1- methyl-1H- indol-5-yl)- thiophen-2- yl]ethyl}- quinazolin-4- amine | 1.00 | 459.2 |
| 50 | 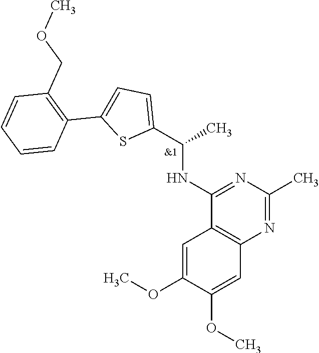 [SMILES: COCc1ccccc1-c1ccc([C@H](C)Nc2nc(C)nc3cc(OC)c(OC)cc23)s1] | 6,7-dimethoxy- N-[1-{5-[2- (methoxy- methyl)phenyl]- thiophen-2-yl}- ethyl]-2-methyl- quinazolin-4- amine | 0.95 | 450.2 |
| 51 | 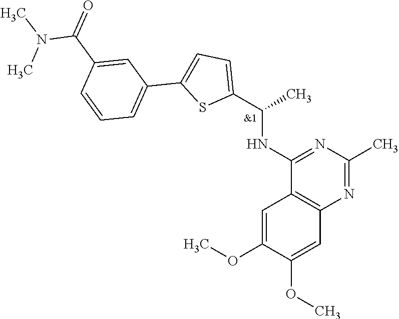 | 3-(5-{1-[(6,7- dimethoxy-2- methylquinazo- lin-4-yl)- amino]ethyl}- thiophen-2-yl)- N,N-dimethyl- benzamide | 0.85 | 477.2 |
| 52 | 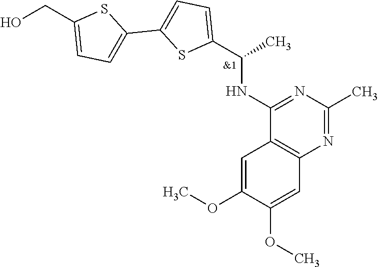 [SMILES: COc1cc2nc(C)nc(N[C@@H](C)c3ccc(-c4ccc(CO)s4)s3)c2cc1OC] | (5′-{1-[(6,7- dimethoxy-2- methylquinazo- lin-4-yl)- amino]ethyl}- 2,2′-bithiophen- 5-yl)methanol | 0.82 | 442.1 |
| 53 | 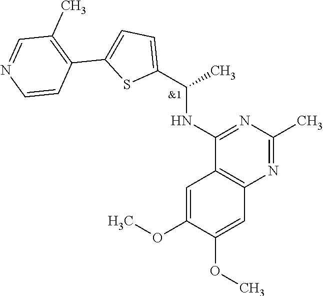 [SMILES: COc1cc2nc(C)nc(N[C@@H](C)c3ccc(-c4ccncc4C)s3)c2cc1OC] | 6,7-dimethoxy- 2-methyl-N-{1- [5-(3-methyl- pyridin-4-yl)- thiophen-2-yl]- ethyl}quinazo- lin-4-amine | 0.63 | 421.2 |
| 54 | 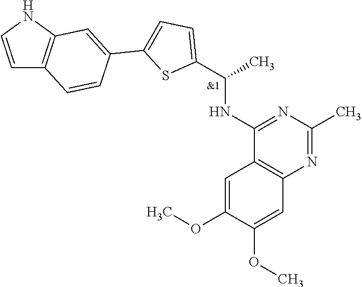 [SMILES: COc1cc2nc(C)nc(N[C@@H](C)c3ccc(-c4ccc5cc[nH]c5c4)s3)c2cc1OC] | N-{1-[5-(1H- indol-6-yl)- thiophen-2-yl]- ethyl}-6,7- dimethoxy-2- methylquinazo- lin-4-amine | 0.95 | 445.2 |
| 55 | 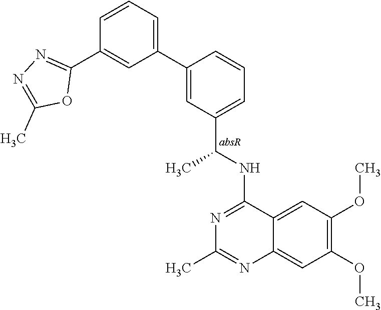 [SMILES: COc1cc2nc(C)nc(N[C@H](C)c3cccc(-c4cccc(-c5nnc(C)o5)c4)c3)c2cc1OC] | 6,7-dimethoxy- 2-methyl-N- {(1R)-1-[3′-(5- methyl-1,3,4- oxadiazol-2- yl)biphenyl-3- yl]ethyl}quinazo- lin-4-amine | 0.91 | 482.2 |
| 56 | 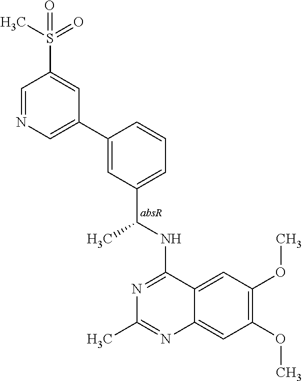 [SMILES: COc1cc2nc(C)nc(N[C@H](C)c3cccc(-c4cncc(S(C)(=O)=O)c4)c3)c2cc1OC] | 6,7-dimethoxy- 2-methyl-N- [(1R)-1-{3-[5- (methylsulfonyl)- pyridin-3-yl]- phenyl}ethyl]- quinazolin-4- amine | 0.78 | 479.2 |
| 57 | 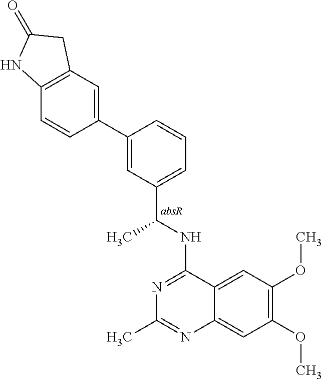 [SMILES: COc1cc2nc(C)nc(N[C@H](C)c3cccc(-c4ccc5c(c4)CC(=O)N5)c3)c2cc1OC] | 5-(3-{(1R)-1- [(6,7-dimethoxy- 2-methylquinazo- lin-4-yl)amino]- ethyl}phenyl)- 1,3-dihydro-2H- indol-2-one | 0.82 | 455.2 |
| 58 | 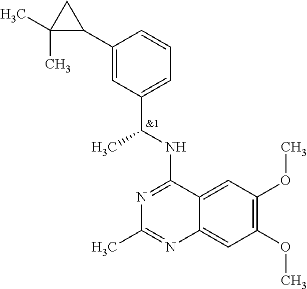 | N-{(1R)-1-[3- (2,2-dimethyl- cyclopropyl)- phenyl]ethyl}- 6,7-dimethoxy- 2-methylquinazo- lin-4-amine | 1.03 | 392.2 |
| 59 | 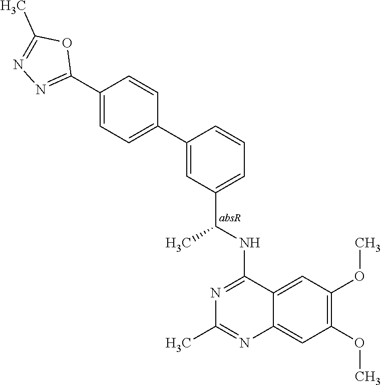 [SMILES: COc1cc2nc(C)nc(N[C@H](C)c3cccc(-c4ccc(-c5nnc(C)o5)cc4)c3)c2cc1OC] | 6,7-dimethoxy- 2-methyl-N- {(1R)-1-[4′-(5- methyl-1,3,4- oxadiazol-2- yl)biphenyl-3- yl]ethyl}quinazo- lin-4-amine | 0.90 | 482.2 |
| 60 | 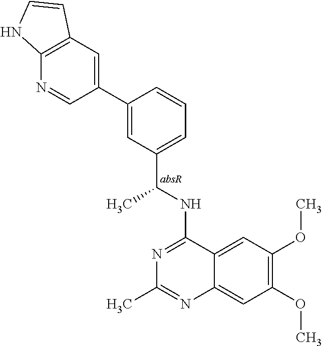 [SMILES: COc1cc2nc(C)nc(N[C@H](C)c3cccc(-c4cnc5[nH]ccc5c4)c3)c2cc1OC] | 6,7-dimethoxy- 2-methyl-N- {(1R)-1-[3-(1H- pyrrolo[2,3-b]- pyridin-5-yl)- phenyl]ethyl}- quinazolin-4- amine | 0.84 | 440.2 |
| 61 | 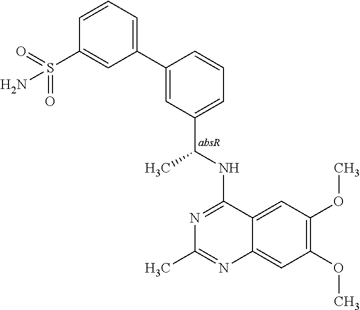 [SMILES: COc1cc2nc(C)nc(N[C@H](C)c3cccc(-c4cccc(S(N)(=O)=O)c4)c3)c2cc1OC] | 3′-{(1R)-1-[(6,7- dimethoxy-2- methylquinazo- lin-4-yl)amino]- ethyl}biphenyl- 3-sulfonamide | 0.81 | 479.2 |
| 62 | 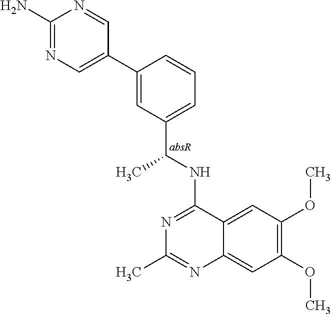 [SMILES: COc1cc2nc(C)nc(N[C@H](C)c3cccc(-c4cnc(N)nc4)c3)c2cc1OC] | N-{(1R)-1-[3-(2- aminopyrimidin- 5-yl)phenyl]- ethyl}-6,7- dimethoxy-2- methylquinazo- lin-4-amine | 0.73 | 417.2 |
| 63 | 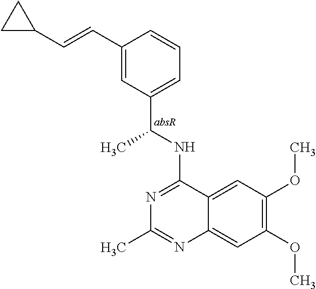 [SMILES: COc1cc2nc(C)nc(N[C@H](C)c3cccc(/C=C/C4CC4)c3)c2cc1OC] | N-[(1R)-1-{3- [(E)-2-cyclo- propylethenyl]- phenyl}ethyl]- 6,7-dimethoxy- 2-methylquinazo- lin-4-amine | 1.00 | 390.2 |
| 64 | 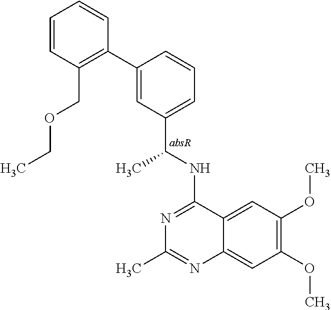 [SMILES: CCOCc1ccccc1-c1cccc([C@@H](C)Nc2nc(C)nc3cc(OC)c(OC)cc23)c1] | N-{(1R)-1-[2′- (ethoxymethyl)- biphenyl-3-yl]- ethyl}-6,7- dimethoxy-2- methylquinazo- lin-4-amine | 1.01 | 458.2 |
| 65 | 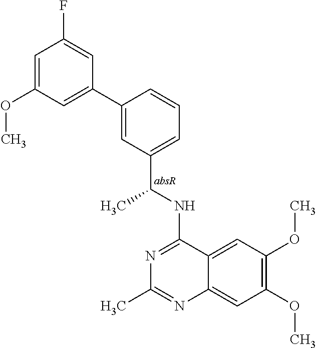 [SMILES: COc1cc(F)cc(-c2cccc([C@@H](C)Nc3nc(C)nc4cc(OC)c(OC)cc34)c2)c1] | N-[(1R)-1-(3′- fluoro-5′- methoxy- biphenyl-3-yl)- ethyl]-6,7- dimethoxy-2- methylquinazo- lin-4-amine | 1.01 | 448.2 |
| 66 | 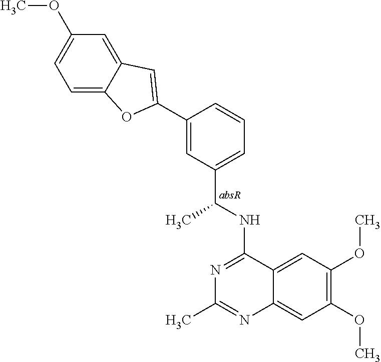 | 6,7-dimethoxy- N-{(1R)-1-[3- (5-methoxy-1- benzofuran-2- yl)phenyl]ethyl}- 2-methylquinazo- lin-4-amine | 1.03 | 470.2 |
| 67 | 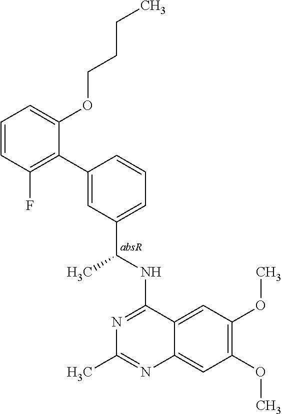 [SMILES: CCCCOc1cccc(F)c1-c1cccc([C@@H](C)Nc2nc(C)nc3cc(OC)c(OC)cc23)c1] | N-[(1R)-1-(2′- butoxy-6′-fluoro- biphenyl-3-yl)- ethyl]-6,7- dimethoxy-2- methylquinazo- lin-4-amine | 1.09 | 490.2 |
| 68 | 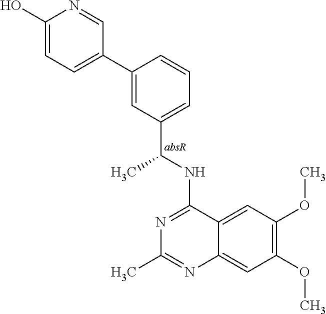 [SMILES: COc1cc2nc(C)nc(N[C@H](C)c3cccc(-c4ccc(O)nc4)c3)c2cc1OC] | 5-(3-{(1R)-1- [(6,7-dimethoxy- 2-methylquinazo- lin-4-yl)amino]- ethyl}phenyl)- pyridin-2-ol | 0.72 | 417.2 |
| 69 | 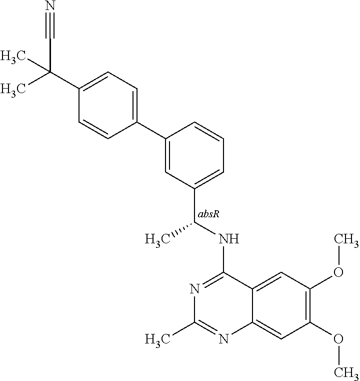 [SMILES: COc1cc2nc(C)nc(N[C@H](C)c3cccc(-c4ccc(C(C)(C)C#N)cc4)c3)c2cc1OC] | 2-(3′-{(1R)-1- [(6,7-dimethoxy- 2-methylquinazo- lin-4-yl)amino]- ethyl}biphenyl- 4-yl)-2-methyl- propanenitrile | 1.00 | 467.2 |
| 70 | 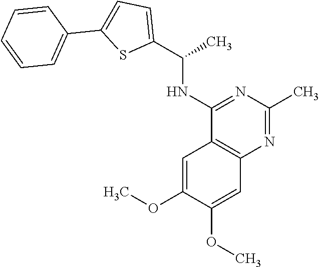 [SMILES: COc1cc2nc(C)nc(N[C@@H](C)c3ccc(-c4ccccc4)s3)c2cc1OC] | 6,7-dimethoxy- 2-methyl-N-[1- (5-phenylthio- phen-2-yl)ethyl]- quinazolin-4- amine | 0.97 | 406.2 |
| 71 | 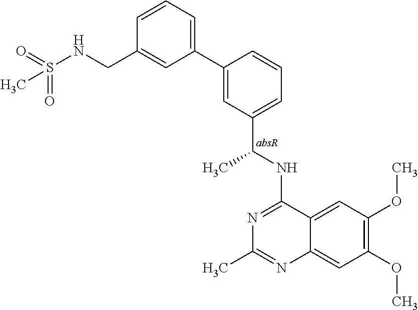 [SMILES: COc1cc2nc(C)nc(N[C@H](C)c3cccc(-c4cccc(CNS(C)(=O)=O)c4)c3)c2cc1OC] | N-[(3′-{(1R)-1- [(6,7-dimethoxy- 2-methylquinazo- lin-4-yl)amino]- ethyl}biphenyl- 3-yl)methyl]- methanesulfon- amide | 0.87 | 507.2 |
| 72 | 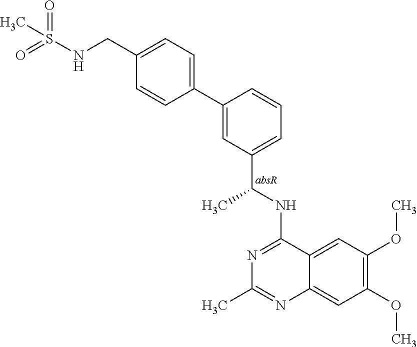 [SMILES: COc1cc2nc(C)nc(N[C@H](C)c3cccc(-c4ccc(CNS(C)(=O)=O)cc4)c3)c2cc1OC] | N-[(3′-{(1R)-1- [(6,7-dimethoxy- 2-methylquinazo- lin-4-yl)amino]- ethyl}biphenyl- 4-yl)methyl]- methanesulfon- amide | 0.86 | 507.2 |
| 73 | 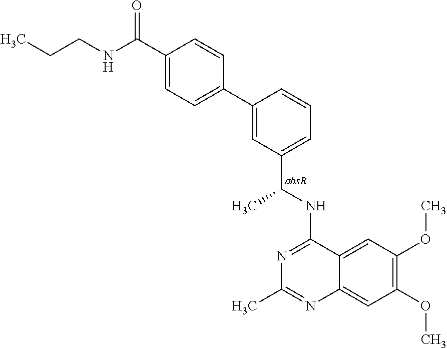 [SMILES: CCCNC(=O)c1ccc(-c2cccc([C@@H](C)Nc3nc(C)nc4cc(OC)c(OC)cc34)c2)cc1] | 3′-{(1R)-1-[(6,7- dimethoxy-2- methylquinazo- lin-4-yl)amino]- ethyl}-N-propyl- biphenyl-4- carboxamide | 0.90 | 485.2 |
| 74 | 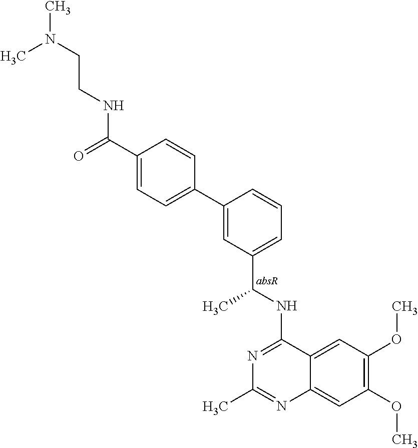 | 3′-{(1R)-1-[(6,7- dimethoxy-2- methylquinazo- lin-4-yl)amino]- ethyl}-N-[2- (dimethylamino)- ethyl]biphenyl- 4-carboxamide | 0.67 | 514.3 |
| 75 | 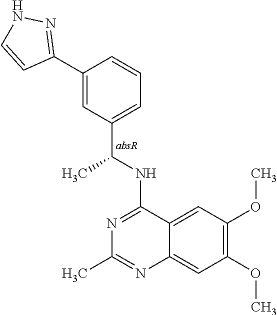 [SMILES: COc1cc2nc(C)nc(N[C@H](C)c3cccc(-c4cc[nH]n4)c3)c2cc1OC] | 6,7-dimethoxy- 2-methyl-N- {(1R)-1-[3-(1H- pyrazol-3-yl)- phenyl]ethyl}- quinazolin-4- amine | 0.78 | 390.2 |
| 76 | 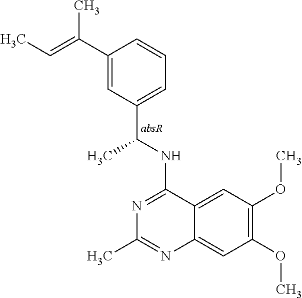 [SMILES: C/C=C(\C)c1cccc([C@@H](C)Nc2nc(C)nc3cc(OC)c(OC)cc23)c1] | N-[(1R)-1-{3- [(2E)-but-2-en- 2-yl]phenyl}- ethyl]-6,7- dimethoxy-2- methylquinazo- lin-4-amine | 0.99 | 378.2 |
| 77 | 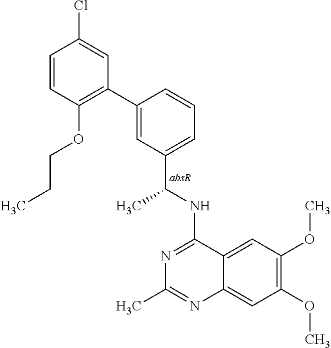 [SMILES: CCCOc1ccc(Cl)cc1-c1cccc([C@@H](C)Nc2nc(C)nc3cc(OC)c(OC)cc23)c1] | N-[(1R)-1-(5′- chloro-2′- propoxybi- phenyl-3- yl)ethyl]-6,7- dimethoxy-2- methylquinazo- lin-4-amine | 1.11 | 492.2 |
| 78 | 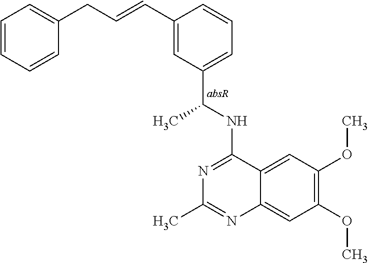 [SMILES: COc1cc2nc(C)nc(N[C@H](C)c3cccc(/C=C/Cc4ccccc4)c3)c2cc1OC] | 6,7-dimethoxy- 2-methyl-N- [(1R)-1-{3- [(1E)-3-phenyl- prop-1-en-1- yl]phenyl}- ethyl]quinazo- lin-4-amine | 1.08 | 440.2 |
| 79 | 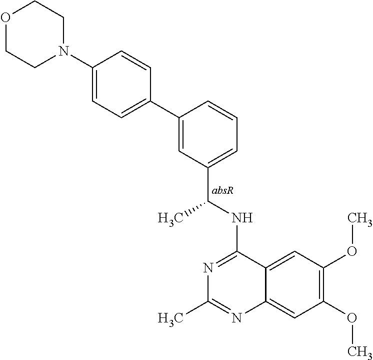 [SMILES: COc1cc2nc(C)nc(N[C@H](C)c3cccc(-c4ccc(N5CCOCC5)cc4)c3)c2cc1OC] | 6,7-dimethoxy- 2-methyl-N- {(1R)-1-[4′- (morpholin- 4-yl)biphenyl- 3-yl]ethyl}- quinazolin-4- amine | 0.95 | 485.2 |
| 80 | 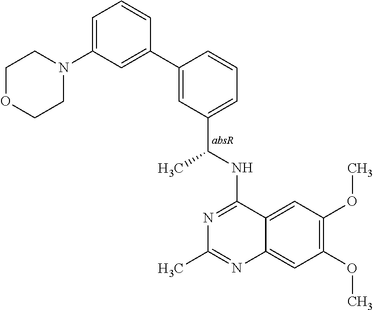 [SMILES: COc1cc2nc(C)nc(N[C@H](C)c3cccc(-c4cccc(N5CCOCC5)c4)c3)c2cc1OC] | 6,7-dimethoxy- 2-methyl-N- {(1R)-1-[3′- (morpholin-4- yl)biphenyl-3- yl]ethyl}- quinazolin-4- amine | 0.95 | 485.2 |
| 81 | 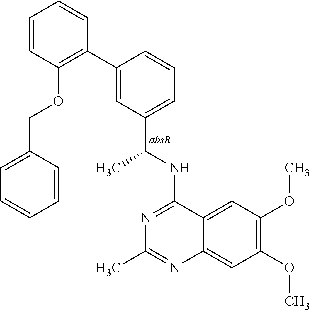 [SMILES: COc1cc2nc(C)nc(N[C@H](C)c3cccc(-c4ccccc4OCc4ccccc4)c3)c2cc1OC] | N-{(1R)-1-[2′- (benzyloxy)- biphenyl-3-yl]- ethyl}-6,7- dimethoxy-2- methylquinazo- lin-4-amine | 1.08 | 506.2 |
| 82 | 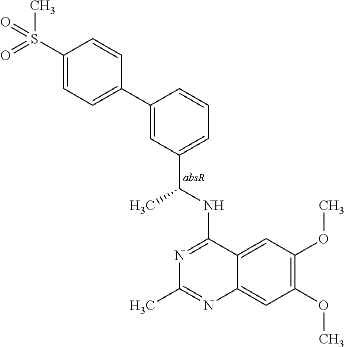 [SMILES: COc1cc2nc(C)nc(N[C@H](C)c3cccc(-c4ccc(S(C)(=O)=O)cc4)c3)c2cc1OC] | 6,7-dimethoxy- 2-methyl-N- {(1R)-1-[4′- (methylsulfonyl)- biphenyl-3-yl]- ethyl}quinazolin- 4-amine | 0.85 | 478.2 |
| 83 | 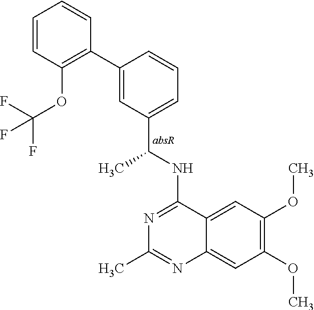 [SMILES: COc1cc2nc(C)nc(N[C@H](C)c3cccc(-c4ccccc4OC(F)(F)F)c3)c2cc1OC] | 6,7-dimethoxy- 2-methyl-N- {(1R)-1-[2′- (trifluorometh- oxy)biphenyl-3- yl]ethyl}quinazo- lin-4-amine | 1.04 | 484.2 |
| 84 | 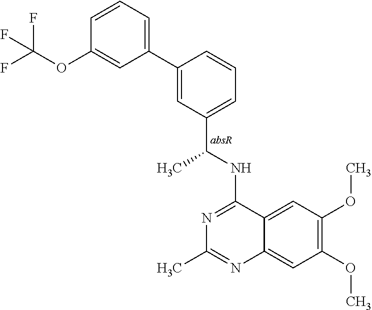 [SMILES: COc1cc2nc(C)nc(N[C@H](C)c3cccc(-c4cccc(OC(F)(F)F)c4)c3)c2cc1OC] | 6,7-dimethoxy- 2-methyl-N- {(1R)-1-[3′- (trifluorometh- oxy)biphenyl-3- yl]ethyl}quinazo- lin-4-amine | 1.07 | 484.2 |
| 85 | 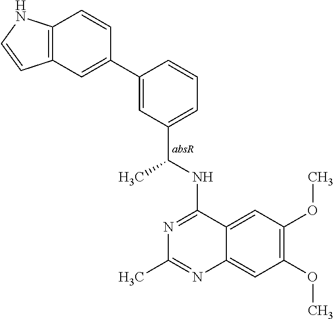 [SMILES: COc1cc2nc(C)nc(N[C@H](C)c3cccc(-c4ccc5[nH]ccc5c4)c3)c2cc1OC] | N-{(1R)-1-[3- (1H-indol-5-yl)- phenyl]ethyl}- 6,7-dimethoxy- 2-methylquinazo- lin-4-amine | 0.94 | 439.2 |
| 86 | 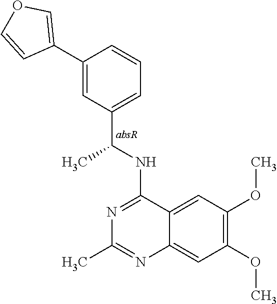 [SMILES: COc1cc2nc(C)nc(N[C@H](C)c3cccc(-c4ccoc4)c3)c2cc1OC] | N-{(1R)-1-[3- (furan-3-yl)- phenyl]ethyl}- 6,7-dimethoxy- 2-methylquinazo- lin-4-amine | 0.91 | 390.2 |
| 87 | 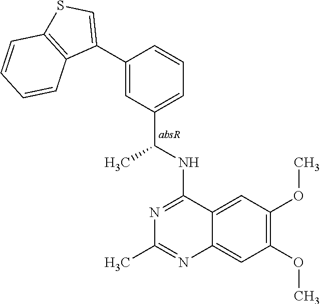 [SMILES: COc1cc2nc(C)nc(N[C@H](C)c3cccc(-c4csc5ccccc45)c3)c2cc1OC] | N-{(1R)-1-[3- (1-benzothio- phen-3-yl)- phenyl]ethyl}- 6,7-dimethoxy- 2-methylquinazo- lin-4-amine | 1.05 | 456.2 |
| 88 | 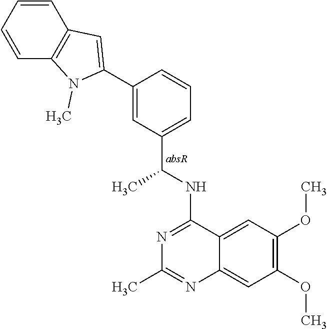 [SMILES: COc1cc2nc(C)nc(N[C@H](C)c3cccc(-c4cc5ccccc5n4C)c3)c2cc1OC] | 6,7-dimethoxy- 2-methyl-N- {(1R)-1-[3-(1- methyl-1H- indol-2-yl)- phenyl]ethyl}- quinazolin-4- amine | 1.04 | 453.2 |
| 89 | 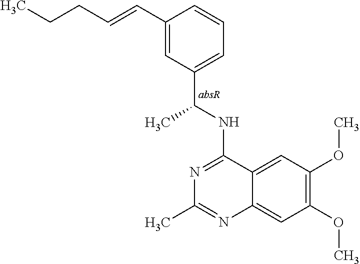 [SMILES: CCC/C=C/c1cccc([C@@H](C)Nc2nc(C)nc3cc(OC)c(OC)cc23)c1] | 6,7-dimethoxy- 2-methyl-N- [(1R)-1-{3- [(1E)-pent-1-en- 1-yl]phenyl}- ethyl]quinazo- lin-4-amine | 1.06 | 392.2 |
| 90 | 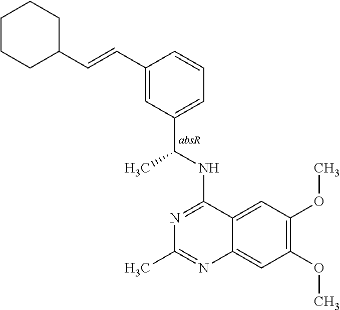 [SMILES: COc1cc2nc(C)nc(N[C@H](C)c3cccc(/C=C/C4CCCCC4)c3)c2cc1OC] | N-[(1R)-1-{3- [(E)-2-cyclo- hexylethenyl]- phenyl}ethyl]- 6,7-dimethoxy- 2-methylquinazo- lin-4-amine | 1.15 | 432.3 |
| 91 | 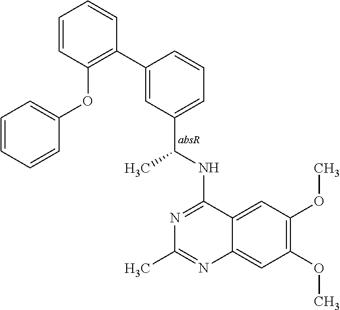 [SMILES: COc1cc2nc(C)nc(N[C@H](C)c3cccc(-c4ccccc4Oc4ccccc4)c3)c2cc1OC] | 6,7-dimethoxy- 2-methyl-N- [(1R)-1-(2′- phenoxybi- phenyl-3-yl)- ethyl]quinazo- lin-4-amine | 1.06 | 492.2 |
| 92 | 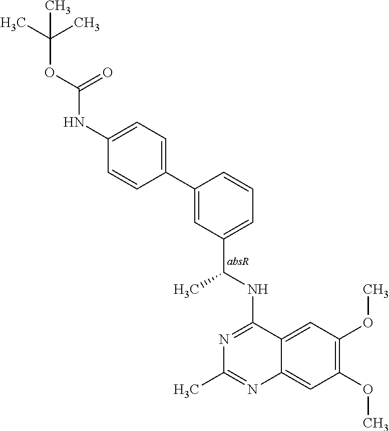 [SMILES: COc1cc2nc(C)nc(N[C@H](C)c3cccc(-c4ccc(NC(=O)OC(C)(C)C)cc4)c3)c2cc1OC] | tert-butyl (3′- {(1R)-1-[(6,7- dimethoxy-2- methylquinazo- lin-4-yl)- amino]ethyl}- biphenyl-4- yl)carbamate | 1.04 | 515.3 |
| 93 | 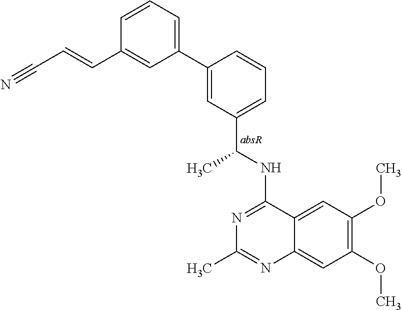 [SMILES: COc1cc2nc(C)nc(N[C@H](C)c3cccc(-c4cccc(/C=C/C#N)c4)c3)c2cc1OC] | (2E)-3-(3′- {(1R)-1-[(6,7- dimethoxy-2- methylquinazo- lin-4-yl)amino]- ethyl}biphenyl- 3-yl)prop-2- enenitrile | 0.97 | 451.2 |
| 94 | 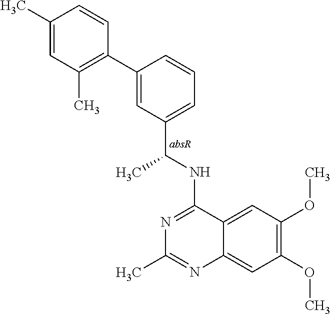 | N-[{1R)-1- (2′,4′-dimethyl- biphenyl-3-yl)- ethyl]-6,7- dimethoxy-2- methylquinazo- lin-4-amine | 1.06 | 428.2 |
| 95 | 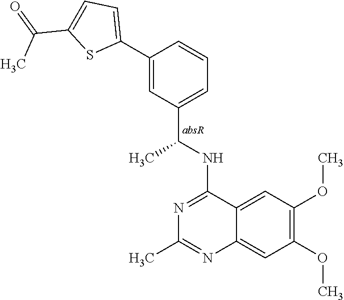 [SMILES: COc1cc2nc(C)nc(N[C@H](C)c3cccc(-c4ccc(C(C)=O)s4)c3)c2cc1OC] | 1-[5-(3-{(1R)-1- [(6,7-dimethoxy- 2-methylquinazo- lin-4-yl)amino]- ethyl}phenyl)- thiophen-2-yl]- ethanone | 0.91 | 448.2 |
| 96 | 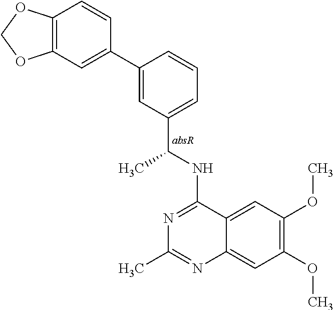 [SMILES: COc1cc2nc(C)nc(N[C@H](C)c3cccc(-c4ccc5c(c4)OCO5)c3)c2cc1OC] | N-{(1R)-1-[3- (1,3-benzodi- oxol-5-yl)- phenyl]ethyl}- 6,7-dimethoxy- 2-methylquinazo- lin-4-amine | 0.96 | 444.2 |
| 97 | 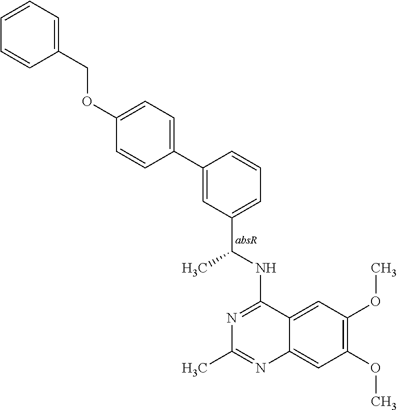 [SMILES: COc1cc2nc(C)nc(N[C@H](C)c3cccc(-c4ccc(OCc5ccccc5)cc4)c3)c2cc1OC] | N-{(1R)-1-[4′- (benzyloxy)- biphenyl-3-yl]- ethyl}-6,7- dimethoxy-2- methylquinazo- lin-4-amine | 1.11 | 506.2 |
| 98 | 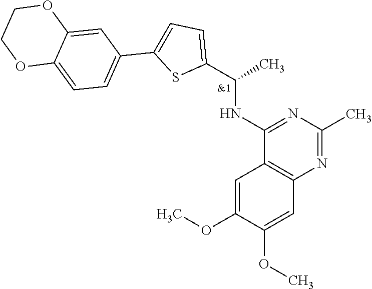 [SMILES: COc1cc2nc(C)nc(N[C@@H](C)c3ccc(-c4ccc5c(c4)OCCO5)s3)c2cc1OC] | N-{1-[5-(2,3- dihydro-1,4- benzodioxin-6- yl)thiophen-2- yl]ethyl}-6,7- dimethoxy-2- methylquinazo- lin-4-amine | 0.95 | 464.2 |
| 99 | 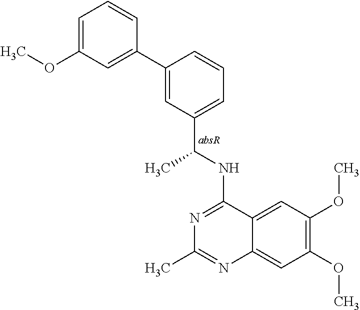 | 6,7-dimethoxy- N-[(1R)-1-(3′- methoxybi- phenyl-3-yl)- ethyl]-2-methyl- quinazolin-4- amine | 0.98 | 430.2 |
| 100 | 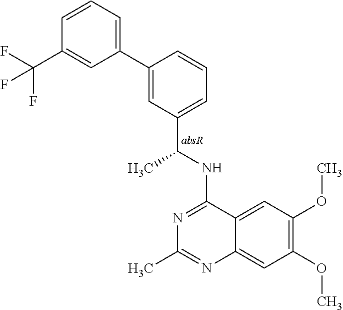 [SMILES: COc1cc2nc(C)nc(N[C@H](C)c3cccc(-c4cccc(C(F)(F)F)c4)c3)c2cc1OC] | 6,7-dimethoxy- 2-methyl-N- {(1R)-1-[3′- (trifluorometh- yl)biphenyl-3- yl]ethyl}quinazo- lin-4-amine | 1.05 | 468.2 |
| 101 | 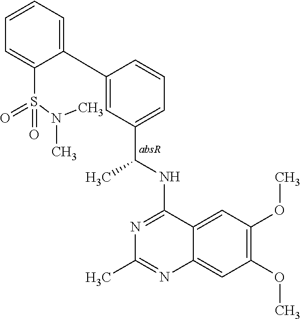 [SMILES: COc1cc2nc(C)nc(N[C@H](C)c3cccc(-c4ccccc4S(=O)(=O)N(C)C)c3)c2cc1OC] | 3′-{(1R)-1-[(6,7- dimethoxy-2- methylquinazo- lin-4-yl)amino]- ethyl}-N,N-di- methylbiphenyl- 2-sulfonamide | 0.91 | 507.2 |
| 102 | 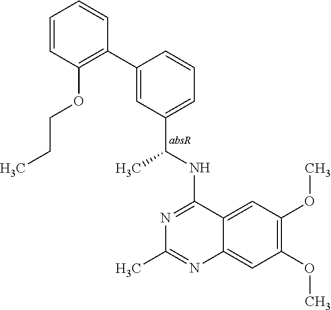 [SMILES: CCCOc1ccccc1-c1cccc([C@@H](C)Nc2nc(C)nc3cc(OC)c(OC)cc23)c1] | 6,7-dimethoxy- 2-methyl-N- [(1R)-1-(2′- propoxybi- phenyl-3-yl)- ethyl]quinazo- lin-4-amine | 1.06 | 458.2 |
| 103 | 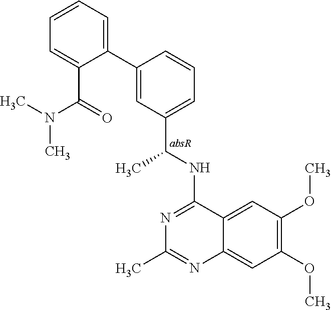 | 3′-{(1R)-1-[(6,7- dimethoxy-2- methylquinazo- lin-4-yl)amino]- ethyl}-N,N-di- methylbiphenyl- 2-carboxamide | 0.86 | 471.2 |
| 104 | 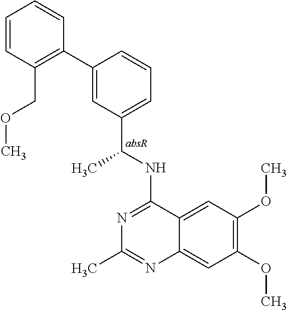 [SMILES: COCc1ccccc1-c1cccc([C@@H](C)Nc2nc(C)nc3cc(OC)c(OC)cc23)c1] | 6,7-dimethoxy- N-{(1R)-1-[2′- (methoxymeth- yl)biphenyl-3- yl]ethyl}-2- methylquinazo- lin-4-amine | 0.96 | 444.2 |
| 105 | 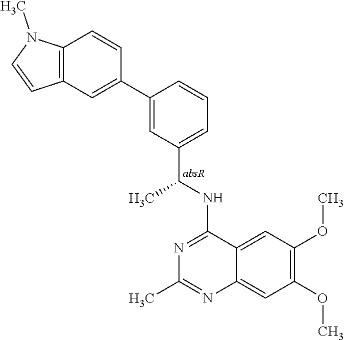 [SMILES: COc1cc2nc(C)nc(N[C@H](C)c3cccc(-c4ccc5c(ccn5C)c4)c3)c2cc1OC] | 6,7-dimethoxy- 2-methyl-N- {(1R)-1-[3-{1- methyl-1H- indol-5-yl)- phenyl]ethyl}- quinazolin-4- amine | 1.01 | 453.2 |
| 106 | 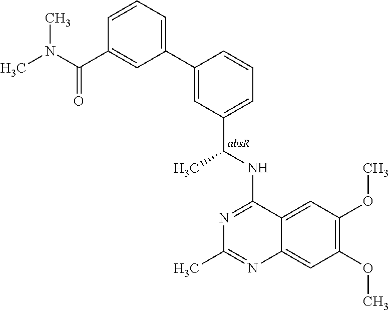 | 3′-{(1R)-1-[(6,7- dimethoxy-2- methylquinazo- lin-4-yl)amino]- ethyl}-N,N-di- methylbiphenyl- 3-carboxamide | 0.86 | 471.2 |
| 107 | 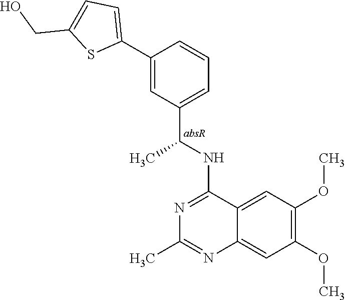 [SMILES: COc1cc2nc(C)nc(N[C@H](C)c3cccc(-c4ccc(CO)s4)c3)c2cc1OC] | [5-(3-{(1R)-1- [(6,7-dimethoxy- 2-methylquinazo- lin-4-yl)amino]- ethyl}phenyl)- thiophen-2-yl]- methanol | 0.84 | 436.2 |
| 108 | 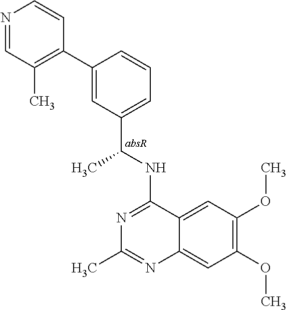 [SMILES: COc1cc2nc(C)nc(N[C@H](C)c3cccc(-c4ccncc4C)c3)c2cc1OC] | 6,7-dimethoxy- 2-methyl-N- {(1R)-1-[3-(3- methylpyridin-4- yl)phenyl]ethyl}- quinazolin-4- amine | 0.64 | 415.2 |
| 109 | 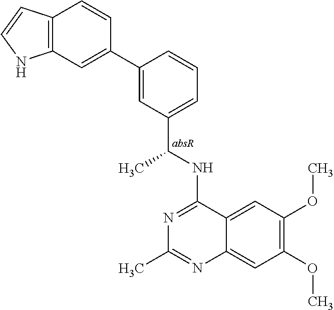 [SMILES: COc1cc2nc(C)nc(N[C@H](C)c3cccc(-c4ccc5cc[nH]c5c4)c3)c2cc1OC] | N-{(1R)-1-[3- (1H-indol-6- yl)phenyl]ethyl}- 6,7-dimethoxy- 2-methylquinazo- lin-4-amine | 0.96 | 439.2 |
| 110 | 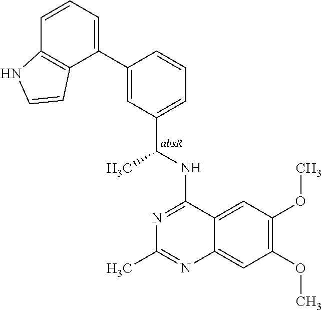 [SMILES: COc1cc2nc(C)nc(N[C@H](C)c3cccc(-c4cccc5[nH]ccc45)c3)c2cc1OC] | N-{(1R)-1-[3- (1H-indol-4-yl)- phenyl]ethyl}- 6,7-dimethoxy- 2-methylquinazo- lin-4-amine | 0.93 | 439.2 |
| 111 | 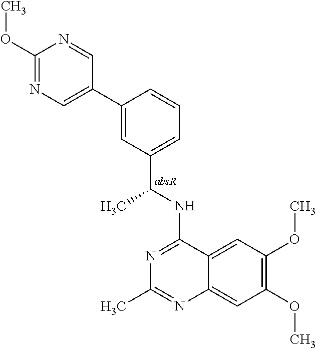 [SMILES: COc1ncc(-c2cccc([C@@H](C)Nc3nc(C)nc4cc(OC)c(OC)cc34)c2)cn1] | 6,7-dimethoxy- N-{(1R)-1-[3- (2-methoxy- pyrimidin-5-yl)- phenyl]ethyl}-2- methylquinazo- lin-4-amine | 0.82 | 432.2 |
| 112 | 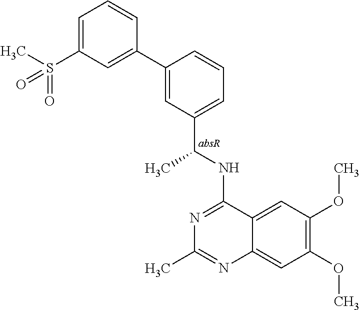 | 6,7-dimethoxy- 2-methyl-N- {(1R)-1-[3′- (methylsulfonyl)- biphenyl-3-yl]- ethyl}quinazolin- 4-amine | 0.85 | 478.2 |
| 113 | 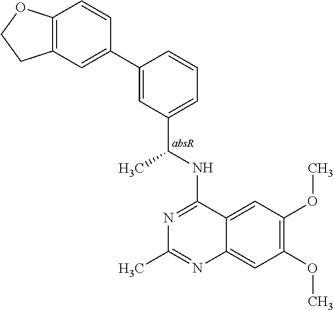 [SMILES: COc1cc2nc(C)nc(N[C@H](C)c3cccc(-c4ccc5c(c4)CCO5)c3)c2cc1OC] | N-{(1R)-1-[3- (2,3-dihydro-1- benzofuran-5- yl)phenyl]ethyl}- 6,7-dimethoxy- 2-methylquinazo- lin-4-amine | 0.97 | 442.2 |
| 114 | 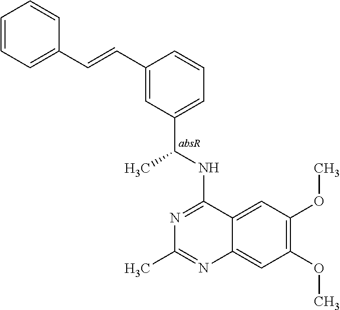 [SMILES: COc1cc2nc(C)nc(N[C@H](C)c3cccc(/C=C/c4ccccc4)c3)c2cc1OC] | 6,7-dimethoxy- 2-methyl-N- [(1R)-1-{3-[(E)- 2-phenylethen- yl]phenyl}ethyl]- quinazolin-4- amine | 1.04 | 426.2 |
| 115 | 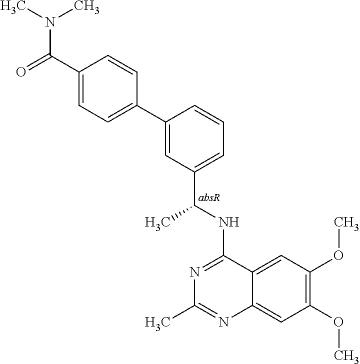 [SMILES: COc1cc2nc(C)nc(N[C@H](C)c3cccc(-c4ccc(C(=O)N(C)C)cc4)c3)c2cc1OC] | 3′-{(1R)-1-[(6,7- dimethoxy-2- methylquinazo- lin-4-yl)amino]- ethyl}-N,N-di- methylbiphenyl- 4-carboxamide | 0.86 | 471.2 |
| 116 | 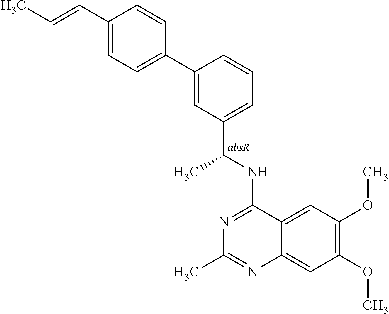 [SMILES: C/C=C/c1ccc(-c2cccc([C@@H](C)Nc3nc(C)nc4cc(OC)c(OC)cc34)c2)cc1] | 6,7-dimethoxy- 2-methyl-N- [(1R)-1-{3- [(1E)-prop-1- en-1-yl]phenyl}- ethyl]quinazolin- 4-amine | 0.95 | 364.2 |
| 117 | 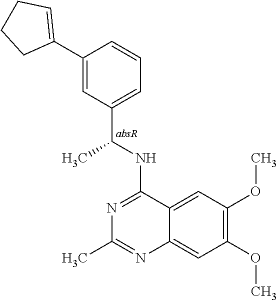 [SMILES: COc1cc2nc(C)nc(N[C@H](C)c3cccc(C4=CCCC4)c3)c2cc1OC] | N-{(1R)-1-[3- (cyclopent-1- en-1-yl)phenyl]- ethyl}-6,7- dimethoxy-2- methylquinazo- lin-4-amine | 1.01 | 390.2 |
| 118 | 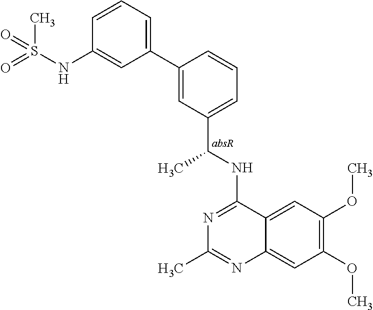 [SMILES: COc1cc2nc(C)nc(N[C@H](C)c3cccc(-c4cccc(NS(C)(=O)=O)c4)c3)c2cc1OC] | N-(3′-{(1R)-1- [(6,7-dimethoxy- 2-methylquinazo- lin-4-yl)amino]- ethyl}biphenyl- 3-yl)methane- sulfonamide | 0.86 | 493.2 |
| 119 | 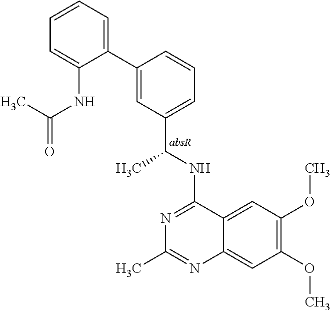 [SMILES: COc1cc2nc(C)nc(N[C@H](C)c3cccc(-c4ccccc4NC(C)=O)c3)c2cc1OC] | N-(3′-{(1R)-1- [(6,7-dimethoxy- 2-methylquinazo- lin-4-yl)amino]- ethyl}biphenyl- 2-yl)acetamide | 0.82 | 457.2 |
| 120 | 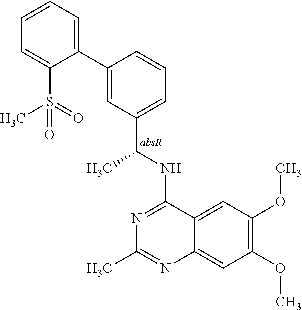 [SMILES: COc1cc2nc(C)nc(N[C@H](C)c3cccc(-c4ccccc4S(C)(=O)=O)c3)c2cc1OC] | 6,7-dimethoxy- 2-methyl-N- {(1R)-1-[2′- (methylsulfonyl)- biphenyl-3-yl]- ethyl}quinazolin- 4-amine | 0.86 | 478.2 |
| 121 | 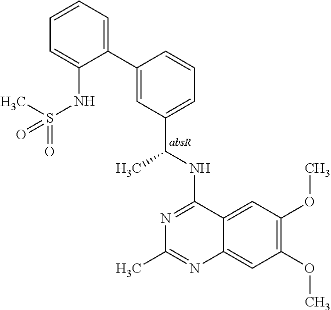 | N-(3′-{(1R)-1- [(6,7-dimethoxy- 2-methylquinazo- lin-4-yl)amino]- ethyl}biphenyl- 2-yl)methane- sulfonamide | 0.86 | 493.2 |
| 122 | 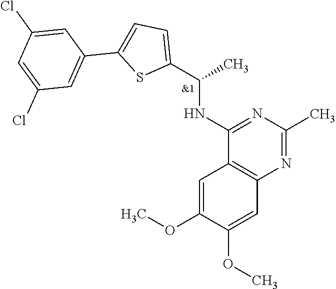 [SMILES: COc1cc2nc(C)nc(N[C@@H](C)c3ccc(-c4cc(Cl)cc(Cl)c4)s3)c2cc1OC] | N-{1-[5-(3,5- dichlorophenyl)- thiophen-2-yl]- ethyl}-6,7- dimethoxy-2- methylquinazo- lin-4-amine | 1.11 | 474.1 |
| 123 | 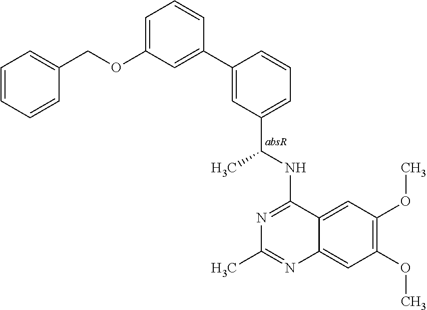 [SMILES: COc1cc2nc(C)nc(N[C@H](C)c3cccc(-c4cccc(OCc5ccccc5)c4)c3)c2cc1OC] | N-{(1R)-1-[3′- (benzyloxy)- biphenyl-3-yl]- ethyl}-6,7- dimethoxy-2- methylquinazo- lin-4-amine | 1.10 | 506.2 |
| 124 | 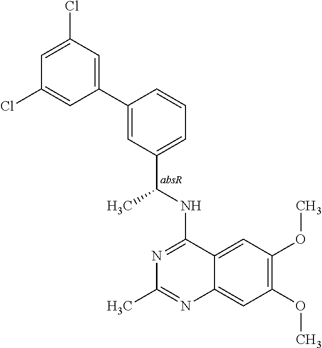 [SMILES: COc1cc2nc(C)nc(N[C@H](C)c3cccc(-c4cc(Cl)cc(Cl)c4)c3)c2cc1OC] | N-[{1R)-1-(3′, 5′-dichlorobi- phenyl-3-yl)- ethyl]-6,7- dimethoxy-2- methylquinazo- lin-4-amine | 1.11 | 468.1 |
| 125 | 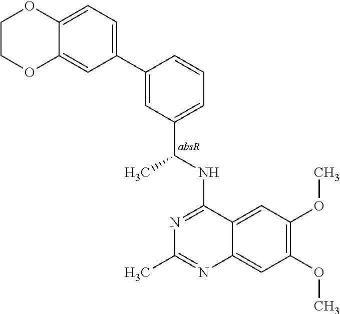 [SMILES: COc1cc2nc(C)nc(N[C@H](C)c3cccc(-c4ccc5c(c4)OCCO5)c3)c2cc1OC] | N-{(1R)-1-[3- (2,3-dihydro- 1,4-benzodioxin- 6-yl)phenyl]- ethyl}-6,7- dimethoxy-2- methylquinazo- lin-4-amine | 0.96 | 458.2 |
| 126 | 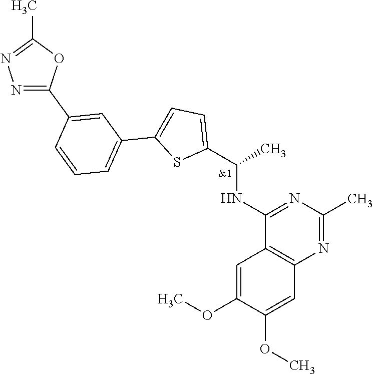 [SMILES: COc1cc2nc(C)nc(N[C@@H](C)c3ccc(-c4cccc(-c5nnc(C)o5)c4)s3)c2cc1OC] | 6,7-dimethoxy- 2-methyl-N-[1- {5-[3-(5-methyl- 1,3,4-oxadiazol- 2-yl)phenyl]- thiophen-2-yl}- ethyl]quinazolin- 4-amine | 0.89 | 488.2 |
| 127 | 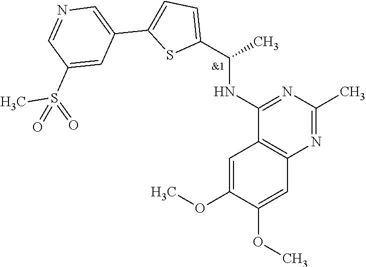 | 6,7-dimethoxy- 2-methyl-N- [1-{5-[5-methyl- sulfonyl)pyridin- 3-yl]thiophen-2- yl}ethyl]quinazo- lin-4-amine | 0.77 | 485.1 |
| 128 | 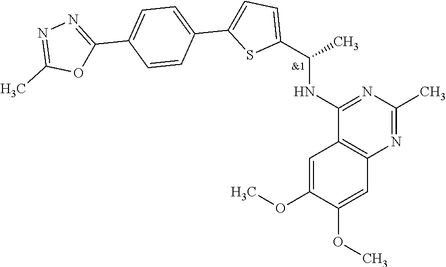 | 6,7-dimethoxy- 2-methyl-N- [1-{5-[4-(5- methyl-1,3,4- oxadiazol-2-yl)- phenyl]thiophen- 2-yl}ethyl]- quinazolin-4- amine | 0.88 | 488.2 |
| 129 | 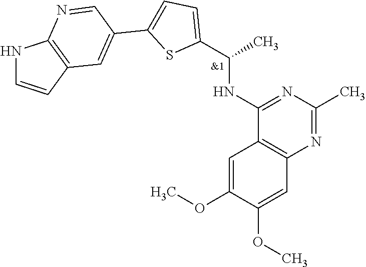 [SMILES: COc1cc2nc(C)nc(N[C@@H](C)c3ccc(-c4cnc5[nH]ccc5c4)s3)c2cc1OC] | 6,7-dimethoxy- 2-methyl-N- {1-[5-(1H- pyrrolo[2,3-b]- pyridin-5-yl)- thiophen-2-yl]- ethyl}quinazolin- 4-amine | 0.84 | 446.2 |
| 130 | 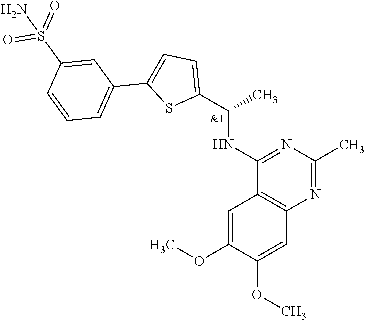 [SMILES: COc1cc2nc(C)nc(N[C@@H](C)c3ccc(-c4cccc(S(N)(=O)=O)c4)s3)c2cc1OC] | 3-(5-{1-[(6,7- dimethoxy-2- methylquinazo- lin-4-yl)amino]- ethyl}thiophen- 2-yl)benzene- sulfonamide | 0.79 | 485.1 |
| 131 | 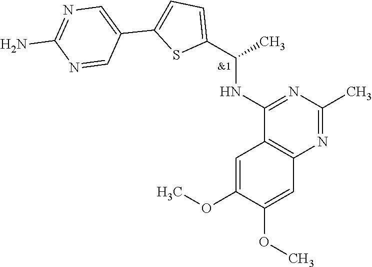 [SMILES: COc1cc2nc(C)nc(N[C@@H](C)c3ccc(-c4cnc(N)nc4)s3)c2cc1OC] | N-{1-[5-(2- aminopyrimidin- 5-yl)thiophen-2- yl]ethyl}-6,7- dimethoxy-2- methylquinazo- lin-4-amine | 0.72 | 423.2 |
| 132 | 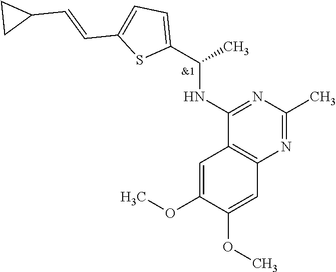 [SMILES: COc1cc2nc(C)nc(N[C@@H](C)c3ccc(/C=C/C4CC4)s3)c2cc1OC] | N-[1-{5-[(E)- 2-cyclopropyl- ethenyl]thiophen- 2-yl}ethyl]-6,7- dimethoxy-2- methylquinazo- lin-4-amine | 0.99 | 396.2 |
| 133 | 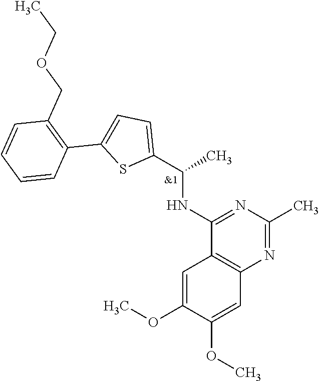 [SMILES: CCOCc1ccccc1-c1ccc([C@H](C)Nc2nc(C)nc3cc(OC)c(OC)cc23)s1] | N-[1-{5-[2- (ethoxymethyl)- phenyl]thiophen- 2-yl}ethyl]-6,7- dimethoxy-2- methylquinazo- lin-4-amine | 1.00 | 464.2 |
| 134 | 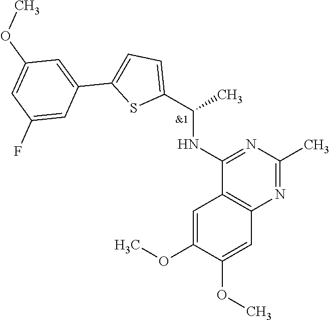 [SMILES: COc1cc(F)cc(-c2ccc([C@H](C)Nc3nc(C)nc4cc(OC)c(OC)cc34)s2)c1] | N-{1-[5-(3- fluoro-5-meth- oxyphenyl)- thiophen-2-yl]- ethyl}-6,7- dimethoxy-2- methylquinazo- lin-4-amine | 1.01 | 454.2 |
| 135 | 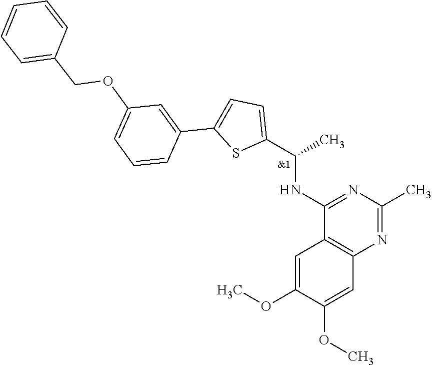 [SMILES: COc1cc2nc(C)nc(N[C@@H](C)c3ccc(-c4cccc(OCc5ccccc5)c4)s3)c2cc1OC] | N-[1-{5-[3- (benzyloxy)- phenyl]thiophen- 2-yl}ethyl]-6,7- dimethoxy-2- methylquinazo- lin-4-amine | 1.10 | 512.2 |
| 136 | 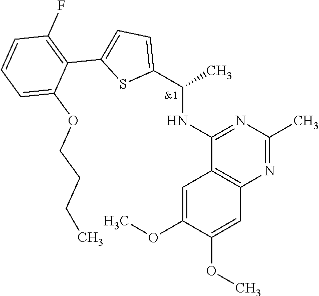 [SMILES: CCCCOc1cccc(F)c1-c1ccc([C@H](C)Nc2nc(C)nc3cc(OC)c(OC)cc23)s1] | N-{1-[5-(2- butoxy-6-fluoro- phenyl)thiophen- 2-yl]ethyl}-6,7- dimethoxy-2- methylquinazo- lin-4-amine | 1.10 | 496.2 |
| 137 | 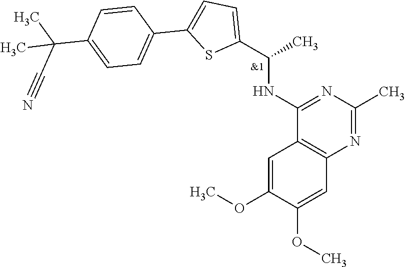 [SMILES: COc1cc2nc(C)nc(N[C@@H](C)c3ccc(-c4ccc(C(C)(C)C#N)cc4)s3)c2cc1OC] | 2-[4-(5-{1-[(6,7- dimethoxy-2- methylquinazo- lin-4-yl)amino]- ethyl}thiophen- 2-yl)phenyl]-2- methylpropane- nitrile | 0.98 | 473.2 |
| 138 | 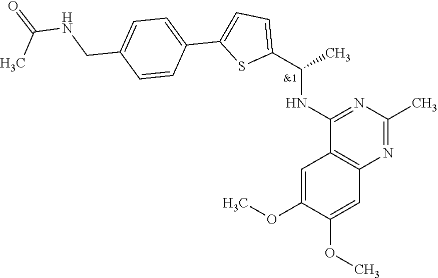 [SMILES: COc1cc2nc(C)nc(N[C@@H](C)c3ccc(-c4ccc(CNC(C)=O)cc4)s3)c2cc1OC] | N-[4-(5-{1- [(6,7-dimethoxy- 2-methylquinazo- lin-4-yl)amino]- ethyl}thiophen- 2-yl)benzyl)- acetamide | 0.81 | 477.2 |
| 139 | 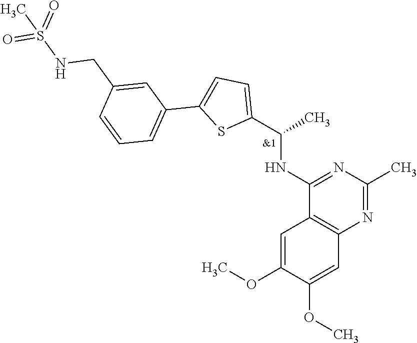 [SMILES: COc1cc2nc(C)nc(N[C@@H](C)c3ccc(-c4cccc(CNS(C)(=O)=O)c4)s3)c2cc1OC] | N-[3-(5-{1-[(6,7- dimethoxy-2- methylquinazo- lin-4-yl)amino]- ethyl}thiophen- 2-yl)benzyl]- methanesulfon- amide | 0.85 | 513.2 |
| 140 | 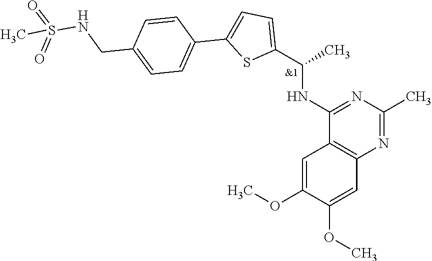 | N-[4-(5-{1- [(6,7-dimethoxy- 2-methylquinazo- lin-4-yl)amino]- ethyl}thiophen- 2-yl)benzyl]- methanesulfon- amide | 0.84 | 513.2 |
| 141 | 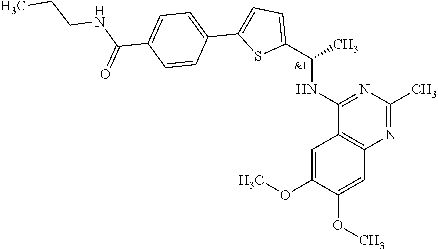 [SMILES: CCCNC(=O)c1ccc(-c2ccc([C@H](C)Nc3nc(C)nc4cc(OC)c(OC)cc34)s2)cc1] | 4-(5-{1-[(6,7- dimethoxy-2- methylquinazo- lin-4-yl)amino]- ethyl}thiophen- 2-yl)-N-propyl- benzamide | 0.89 | 491.2 |
| 142 | 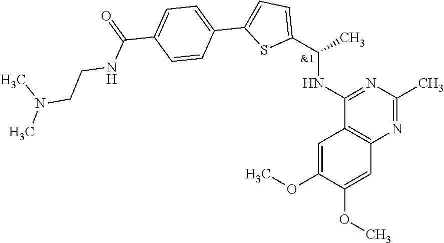 | 4-(5-{1-[(6,7- dimethoxy-2- methylquinazo- lin-4-yl)amino]- ethyl}thiophen- 2-yl)-N-[2- (dimethylamino)- ethyl]benzamide | 0.65 | 520.2 |
| 143 | 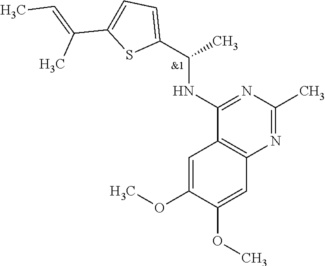 [SMILES: C/C=C(\C)c1ccc([C@H](C)Nc2nc(C)nc3cc(OC)c(OC)cc23)s1] | N-[1-{5-[(2E)- but-2-en-2-yl]- thiophen-2-yl}- ethyl]-6,7- dimethoxy-2- methylquinazo- lin-4-amine | 0.98 | 384.2 |
| 144 | 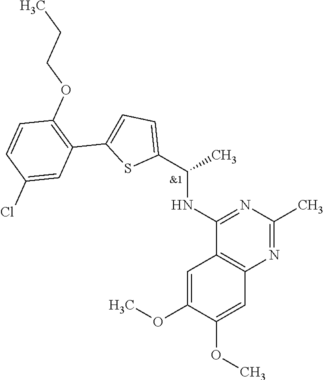 [SMILES: CCCOc1ccc(Cl)cc1-c1ccc([C@H](C)Nc2nc(C)nc3cc(OC)c(OC)cc23)s1] | N-{1-[5-(5- chloro-2- propoxyphenyl)- thiophen-2-yl]- ethyl}-6,7- dimethoxy-2- methylquinazo- lin-4-amine | 1.11 | 498.2 |
| 145 | 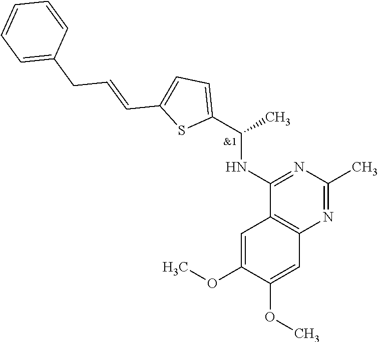 [SMILES: COc1cc2nc(C)nc(N[C@@H](C)c3ccc(/C=C/Cc4ccccc4)s3)c2cc1OC] | 6,7-dimethoxy- 2-methyl-N- [1-{5-[(1E)-3- phenylprop-1- en-1-yl]thiophen- 2-yl}ethyl]- quinazolin-4- amine | 1.07 | 446.2 |
| 146 | 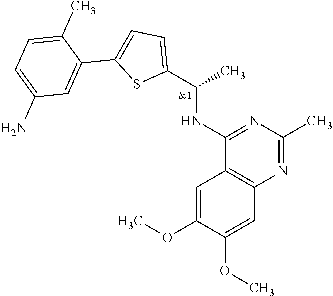 [SMILES: COc1cc2nc(C)nc(N[C@@H](C)c3ccc(-c4cc(N)ccc4C)s3)c2cc1OC] | N-{1-[5-(5- amino-2-methyl- phenyl)thiophen- 2-yl]ethyl}-6,7- dimethoxy-2- methylquinazo- lin-4-amine | 0.80 | 435.2 |
| 147 | 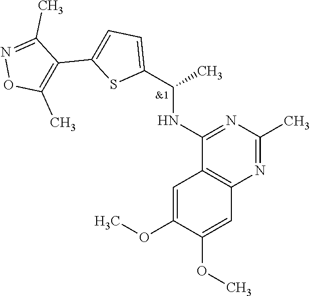 [SMILES: COc1cc2nc(C)nc(N[C@@H](C)c3ccc(-c4c(C)noc4C)s3)c2cc1OC] | N-{1-[5-(3,5- dimethyl-1,2- oxazol-4-yl)- thiophen-2-yl]- ethyl}-6,7- dimethoxy-2- methylquinazo- lin-4-amine | 0.86 | 425.2 |
| 148 | 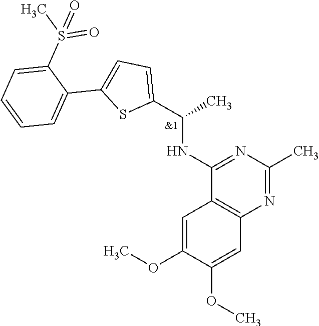 [SMILES: COc1cc2nc(C)nc(N[C@@H](C)c3ccc(-c4ccccc4S(C)(=O)=O)s3)c2cc1OC] | 6,7-dimethoxy- 2-methyl-N-[1- {5-[2-(methyl- sulfonyl)phenyl]- thiophen-2-yl}- ethyl]quinazolin- 4-amine | 0.84 | 484.1 |
| 149 | 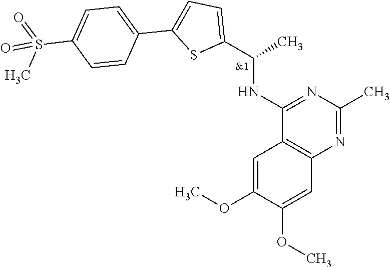 [SMILES: COc1cc2nc(C)nc(N[C@@H](C)c3ccc(-c4ccc(S(C)(=O)=O)cc4)s3)c2cc1OC] | 6,7-dimethoxy- 2-methyl-N-[1- {5-[4-(methyl- sulfonyl)phenyl]- thiophen-2-yl}- ethyl]quinazolin- 4-amine | 0.83 | 484.1 |
| 150 | 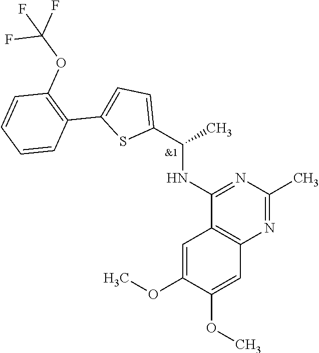 [SMILES: COc1cc2nc(C)nc(N[C@@H](C)c3ccc(-c4ccccc4OC(F)(F)F)s3)c2cc1OC] | 6,7-dimethoxy- 2-methyl-N- [1-{5-[2-(tri- fluoromethoxy)- phenyl]thiophen- 2-yl}ethyl]- quinazolin-4- amine | 1.04 | 490.1 |
| 151 | 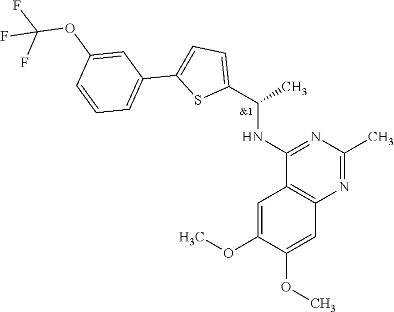 [SMILES: COc1cc2nc(C)nc(N[C@@H](C)c3ccc(-c4cccc(OC(F)(F)F)c4)s3)c2cc1OC] | 6,7-dimethoxy- 2-methyl-N- [1-{5-[3-(tri- fluoromethoxy)- phenyl]thiophen- 2-yl}ethyl]- quinazolin-4- amine | 1.07 | 490.1 |
| 152 | 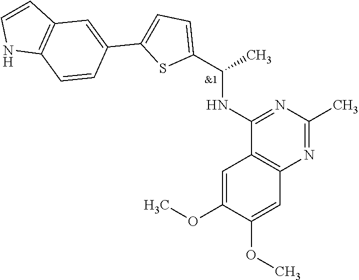 [SMILES: COc1cc2nc(C)nc(N[C@@H](C)c3ccc(-c4ccc5[nH]ccc5c4)s3)c2cc1OC] | N-{1-[5-(1H- indol-5-yl)- thiophen-2-yl]- ethyl}-6,7- dimethoxy-2- methylquinazo- lin-4-amine | 0.92 | 445.2 |
| 153 | 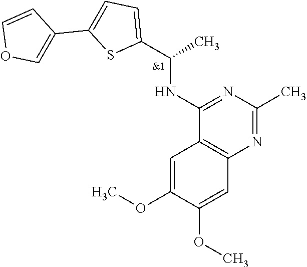 [SMILES: COc1cc2nc(C)nc(N[C@@H](C)c3ccc(-c4ccoc4)s3)c2cc1OC] | N-{1-[5-(furan- 3-yl)thiophen-2- yl]ethyl}-6,7- dimethoxy-2- methylquinazo- lin-4-amine | 0.90 | 396.1 |
| 154 | 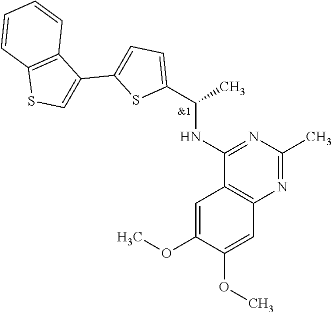 [SMILES: COc1cc2nc(C)nc(N[C@@H](C)c3ccc(-c4csc5ccccc45)s3)c2cc1OC] | N-{1-[5-(1- benzothiophen- 3-yl)thiophen-2- yl]ethyl}-6,7- dimethoxy-2- methylquinazo- lin-4-amine | 1.05 | 462.1 |
| 155 | 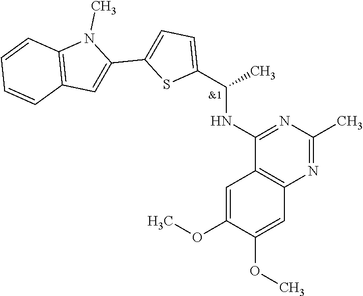 [SMILES: COc1cc2nc(C)nc(N[C@@H](C)c3ccc(-c4cc5ccccc5n4C)s3)c2cc1OC] | 6,7-dimethoxy- 2-methyl-N-{1- [5-(1-methyl- 1H-indol-2-yl)- thiophen-2-yl]- ethyl}quinazolin- 4-amine | 1.03 | 459.2 |
| 156 | 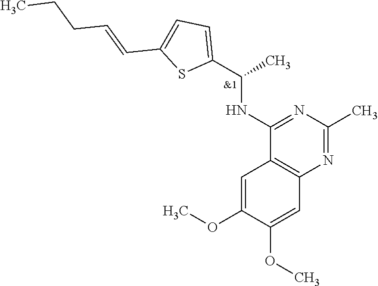 [SMILES: CCC/C=C/c1ccc([C@H](C)Nc2nc(C)nc3cc(OC)c(OC)cc23)s1] | 6,7-dimethoxy- 2-methyl-N- [1-{5-[(1E)-pent- 1-en-1-yl]- thiophen-2-yl}- ethyl]quinazolin- 4-amine | 1.05 | 398.2 |
| 157 | 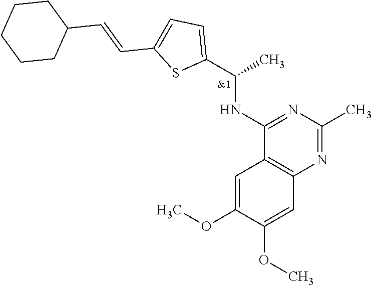 [SMILES: COc1cc2nc(C)nc(N[C@@H](C)c3ccc(/C=C/C4CCCCC4)s3)c2cc1OC] | N-[1-{5-[(E)-2- cyclohexyl- ethenyl]thiophen- 2-yl}ethyl]-6,7- dimethoxy-2- methylquinazo- lin-4-amine | 1.15 | 438.2 |
| 158 | 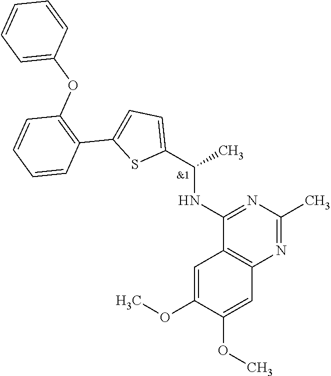 [SMILES: COc1cc2nc(C)nc(N[C@@H](C)c3ccc(-c4ccccc4Oc4ccccc4)s3)c2cc1OC] | 6,7-dimethoxy- 2-methyl-N- {1-[5-(2-phen- oxyphenyl)thio- phen-2-yl]ethyl}- quinazolin-4- amine | 1.08 | 498.2 |
| 159 | 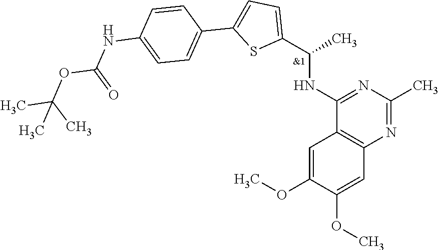 [SMILES: COc1cc2nc(C)nc(N[C@@H](C)c3ccc(-c4ccc(NC(=O)OC(C)(C)C)cc4)s3)c2cc1OC] | tert-butyl [4- (5-{1-[(6,7- dimethoxy-2- methylquinazo- lin-4-yl)amino]- ethyl}thiophen- 2-yl)phenyl]- carbamate | 1.03 | 521.2 |
| 160 | 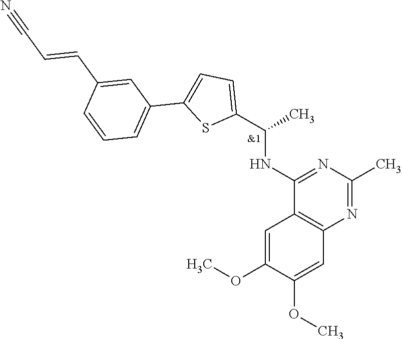 [SMILES: COc1cc2nc(C)nc(N[C@@H](C)c3ccc(-c4cccc(/C=C/C#N)c4)s3)c2cc1OC] | (2E)-3-[3-(5-{1- [(6,7-dimethoxy- 2-methylquinazo- lin-4-yl)amino]- ethyl}thiophen- 2-yl)phenyl]- prop-2-enenitrile | 0.96 | 457.2 |
| 161 | 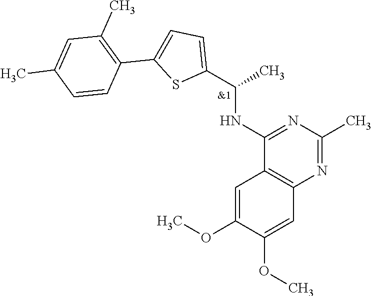 | N-{1-[5-(2,4- dimethylphenyl)- thiophen-2-yl]- ethyl}-6,7- dimethoxy-2- methylquinazo- lin-4-amine | 1.06 | 434.2 |
| 162 | 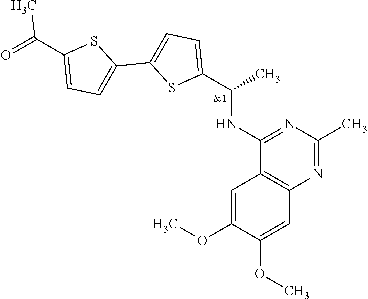 [SMILES: COc1cc2nc(C)nc(N[C@@H](C)c3ccc(-c4ccc(C(C)=O)s4)s3)c2cc1OC] | 1-(5′-{1-[(6,7- dimethoxy-2- methylquinazo- lin-4-yl)amino]- ethyl}-2,2′- bithiophen-5- yl)ethanone | 0.89 | 454.1 |
| 163 | 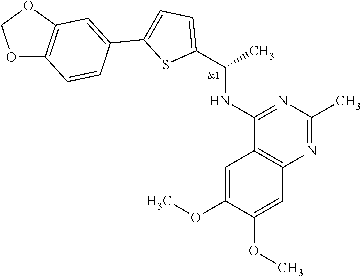 [SMILES: COc1cc2nc(C)nc(N[C@@H](C)c3ccc(-c4ccc5c(c4)OCO5)s3)c2cc1OC] | N-{1-[5-(1,3- benzodioxol- 5-yl)thiophen- 2-yl]ethyl}-6,7- dimethoxy-2- methylquinazo- lin-4-amine | 0.96 | 450.1 |
| 164 | 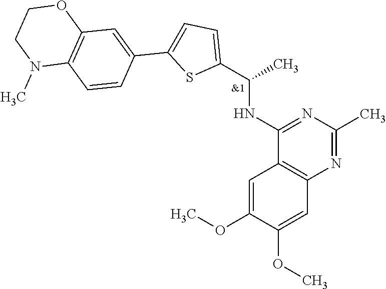 [SMILES: COc1cc2nc(C)nc(N[C@@H](C)c3ccc(-c4ccc5c(c4)OCCN5C)s3)c2cc1OC] | 6,7-dimethoxy- 2-methyl-N-{1- [5-(4-methyl- 3,4-dihydro-2H- 1,4-benzoxazin- 7-yl)thiophen- 2-yl]ethyl}- quinazolin-4- amine | 0.98 | 477.2 |
| 165 | 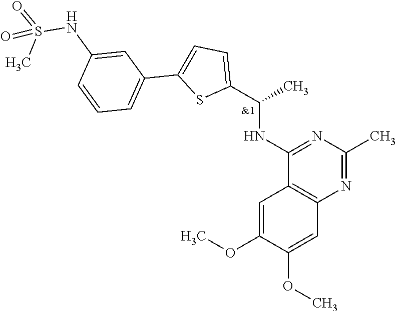 [SMILES: COc1cc2nc(C)nc(N[C@@H](C)c3ccc(-c4cccc(NS(C)(=O)=O)c4)s3)c2cc1OC] | N-[3-(5-{1- [(6,7-dimethoxy- 2-methylquinazo- lin-4-yl)amino]- ethyl}thiophen- 2-yl)phenyl]- methanesulfon- amide | 0.85 | 499.1 |
| 166 | 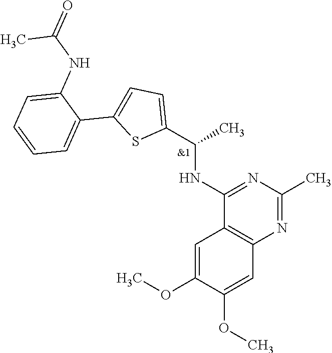 [SMILES: COc1cc2nc(C)nc(N[C@@H](C)c3ccc(-c4ccccc4NC(C)=O)s3)c2cc1OC] | N-[2-(5-{1- [(6,7-dimethoxy- 2-methylquinazo- lin-4-yl)amino]- ethyl}thiophen- 2-yl)phenyl]- acetamide | 0.80 | 463.2 |
| 167 | 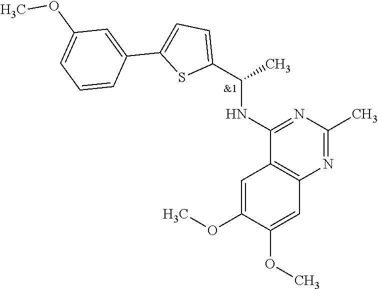 | 6,7-dimethoxy- N-{1-[5-(3- methoxyphenyl)- thiophen-2-yl]- ethyl}-2-methyl- quinazolin-4- amine | 0.97 | 436.2 |
| 168 | 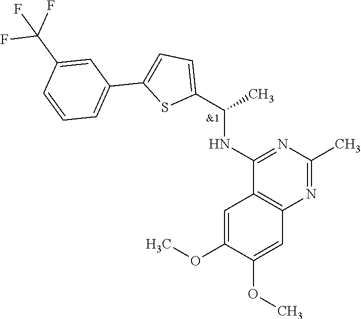 [SMILES: COc1cc2nc(C)nc(N[C@@H](C)c3ccc(-c4cccc(C(F)(F)F)c4)s3)c2cc1OC] | 6,7-dimethoxy- 2-methyl-N-[1- {5-[3-(trifluoro- methyl)phenyl]- thiophen-2-yl}- ethyl]quinazolin- 4-amine | 1.05 | 474.1 |
| 169 | 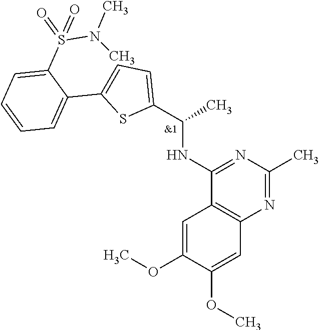 [SMILES: COc1cc2nc(C)nc(N[C@@H](C)c3ccc(-c4ccccc4S(=O)(=O)N(C)C)s3)c2cc1OC] | 2-(5-{1-[(6,7- dimethoxy-2- methylquinazo- lin-4-yl)amino]- ethyl}thiophen- 2-yl)-N,N-di- methylbenzene- sulfonamide | 0.90 | 513.2 |
| 170 | 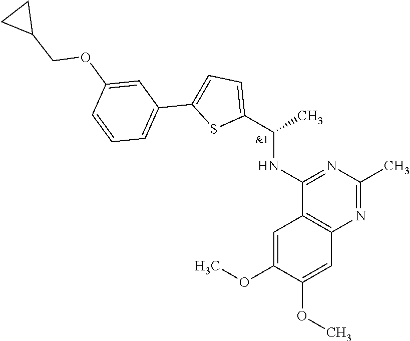 [SMILES: COc1cc2nc(C)nc(N[C@@H](C)c3ccc(-c4cccc(OCC5CC5)c4)s3)c2cc1OC] | N-[1-{5-[3- (cyclopropyl- methoxy)- phenyl]thiophen- 2-yl}ethyl]-6,7- dimethoxy-2- methylquinazo- lin-4-amine | 1.07 | 476.2 |
| 171 | 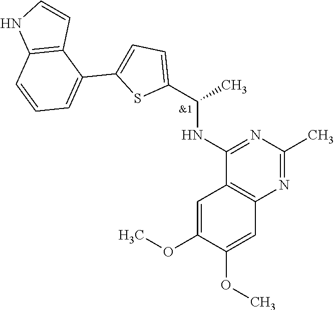 [SMILES: COc1cc2nc(C)nc(N[C@@H](C)c3ccc(-c4cccc5[nH]ccc45)s3)c2cc1OC] | N-{1-[5-(1H- indol-4-yl)- thiophen-2-yl]- ethyl}-6,7- dimethoxy-2- methylquinazo- lin-4-amine | 0.92 | 445.2 |
| 172 | 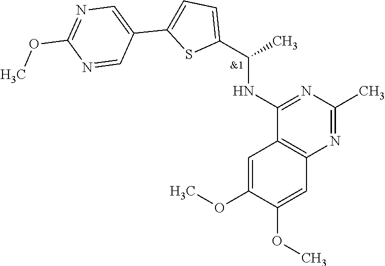 | 6,7-dimethoxy- N-{1-[5-(2- methoxypyrim- idin-5-yl)- thiophen-2- yl]ethyl}-2- methylquinazo- lin-4-amine | 0.81 | 438.2 |
| 173 | 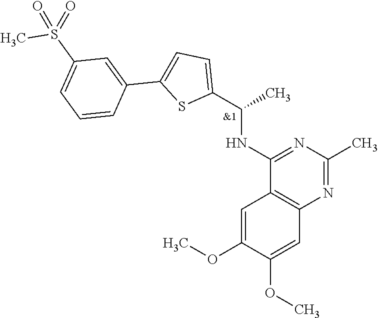 | 6,7-dimethoxy- 2-methyl-N-[1- {5-[3-(methyl- sulfonyl)phenyl]- thiophen-2-yl}- ethyl]quinazolin- 4-amine | 0.83 | 484.1 |
| 174 | 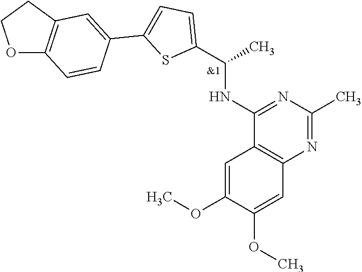 [SMILES: COc1cc2nc(C)nc(N[C@@H](C)c3ccc(-c4ccc5c(c4)CCO5)s3)c2cc1OC] | N-{1-[5-(2,3- dihydro-1- benzofuran-5- yl)thiophen-2- yl]ethyl}-6,7- dimethoxy-2- methylquinazo- lin-4-amine | 0.97 | 448.2 |
| 175 | 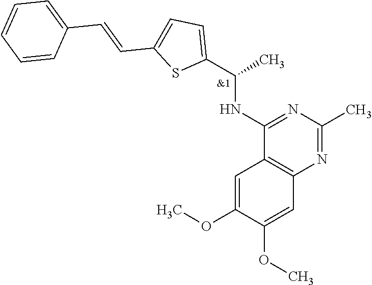 [SMILES: COc1cc2nc(C)nc(N[C@@H](C)c3ccc(/C=C/c4ccccc4)s3)c2cc1OC] | 6,7-dimethoxy- 2-methyl-N-[1- {5-[(E)-2- phenylethenyl]- thiophen-2-yl}- ethyl]quinazolin- 4-amine | 1.04 | 432.2 |
| 176 | 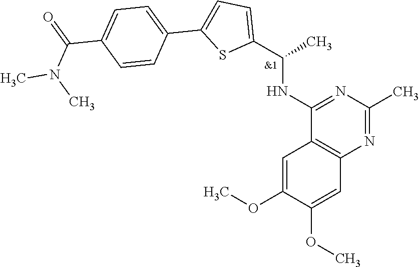 | 4-(5-{1-[(6,7- dimethoxy-2- methylquinazo- lin-4-yl)amino]- ethyl}thiophen- 2-yl)-N,N- dimethylbenz- amide | 0.84 | 477.2 |
| 177 | 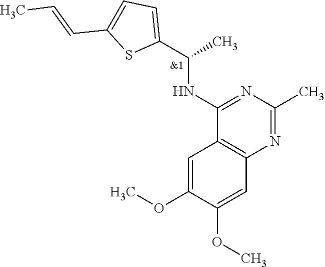 [SMILES: C/C=C/c1ccc([C@H](C)Nc2nc(C)nc3cc(OC)c(OC)cc23)s1] | 6,7-dimethoxy- 2-methyl-N-[1- {5-[(1E)-prop- 1-en-1-yl]- thiophen-2-yl}- ethyl]quinazolin- 4-amine | 0.94 | 370.2 |

### Example 178

### methyl 2-(5-{1-[(6,7-dimethoxy-2-methylquinazolin-4-yl)amino]ethyl}thiophen-2-yl)benzoate

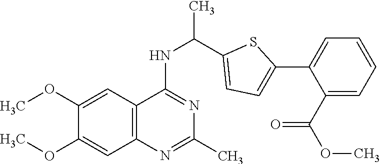 [SMILES: COC(=O)c1ccccc1-c1ccc(C(C)Nc2nc(C)nc3cc(OC)c(OC)cc23)s1]

Under argon, N-[1-(5-bromothiophen-2-yl)ethyl]-6,7-dimethoxy-2-methylquinazolin-4-amine (described in example 209; 200 mg, 85% purity, 416 μmol), [2-(methoxycarbonyl)phenyl]boronic acid (commercially available; 74.9 mg, 416 μmol), K₂CO₃ (230 mg, 1.67 mmol) and Pd(PPh₃)₄ (48.1 mg, 41.6 μmol) in dioxane (4.0 mL) were stirred at 110° C. overnight. H₂O was added, the mixture extracted with DCM and the solvent removed in vacuo. Purification by preparative HPLC (basic conditions) gave the title compound as a white solid (61.5 mg, 36%). ¹H-NMR (400 MHz, DMSO-d₆): δ [ppm]=8.14 (d, 1H), 7.64 (s, 1H), 7.61 7.53 (m, 2H), 7.52-7.47 (m, 1H), 7.45-7.40 (m, 1H), 7.05 (s, 2H), 6.95 (d, 1H), 5.93 (quin, 1H), 3.87 (s, 6H), 3.62 (s, 3H), 2.43 (s, 3H), 1.71 (d, 3H). LC-MS (method 7): m/z: [M+H]⁺=464, Rt=0.96 min.

### Example 179

### 1-[2-(5-{1-[(6,7-dimethoxy-2-methylquinazolin-4-yl)amino]ethyl}thiophen-2-yl)benzyl]piperidine-4-carboxamide

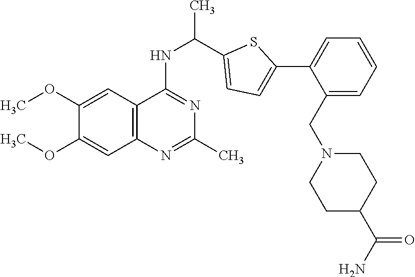 [SMILES: COc1cc2nc(C)nc(NC(C)c3ccc(-c4ccccc4CN4CCC(C(N)=O)CC4)s3)c2cc1OC]

### Step a

### 2-(5-{1-[(6,7-di methoxy-2-methylquinazolin-4-yl)amino]ethyl}-2-thienyl)benzaldehyde

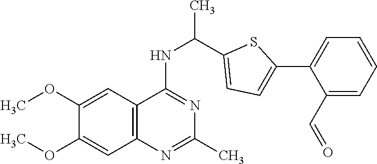 [SMILES: COc1cc2nc(C)nc(NC(C)c3ccc(-c4ccccc4C=O)s3)c2cc1OC]

The title compound was prepared in analogy to example 178 using N-[1-(5-bromothiophen-2-yl)ethyl]-6,7-dimethoxy-2-methylquinazolin-4-amine (described in example 209; 10 g, 22.4 mmol) and 2-formylphenylboronic acid (commercially available; 3.37 g, 22.4 mmol) to give the title compound in 79% yield (8.4 g). The crude product was recrystallized from diethylether and used directly in the next step.

### Step b

### 1-[2-(5-{1-[(6,7-dimethoxy-2-methylquinazolin-4-yl)amino]ethyl}thiophen-2-yl)benzyl]piperid ne-4-carboxamide

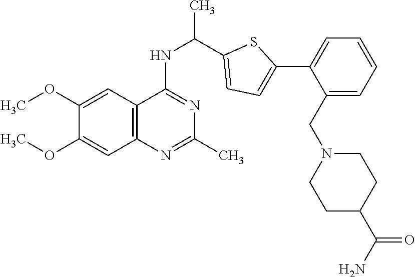 [SMILES: COc1cc2nc(C)nc(NC(C)c3ccc(-c4ccccc4CN4CCC(C(N)=O)CC4)s3)c2cc1OC]

To 2-(5-{1-[(6,7-dimethoxy-2-methylquinazolin-4-yl)amino]ethyl}thiophen-2-yl)benzaldehyde (50.0 mg, 115 μmol), piperidine-4-carboxamide (29.6 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature during 5 hours. The reaction was quenched with aqueous NaOH (1.0 M, 1.0 mL), the mixture extracted with DCM and the solvent removed in vacuo. Purification by preparative HPLC (acidic conditions) gave the title compound as a white solid (19.5 mg, 31%). ¹H-NM R (400 MHz, DMSO-d₆): δ [ppm]=8.16 (d, 1H), 7.65 (s, 1H), 7.43 7.39 (m, 1H), 7.38-7.35 (m, 1H), 7.33-7.25 (m, 2H), 7.21 (d, 1H), 7.19 (br s, 1H), 7.08 (dd, 1H), 7.04 (s, 1H), 6.71 (br s, 1H), 5.97 (quin, 1H), 3.87 (s, 6H), 3.39 (s, 2H), 2.76 (br t, 2H), 2.43 (s, 3H), 2.07-1.97 (m, 1H), 1.87 (br t, 2H), 1.71 (d, 3H), 1.63-1.55 (m, 2H), 1.54-1.42 (m, 2H). LC-MS (method 7): m/z: [M+H]⁺=546, Rt=0.58 min.

### Example 180

### 2-[2-(5-{1-[(6,7-dimethoxy-2-methylquinazolin-4-yl)amino]ethyl}thiophen-2-yl)phenyl]ethanol

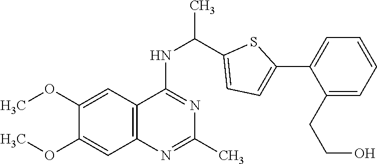 [SMILES: COc1cc2nc(C)nc(NC(C)c3ccc(-c4ccccc4CCO)s3)c2cc1OC]

Under argon, N-[1-(5-bromothiophen-2-yl)ethyl]-6,7-dimethoxy-2-methylquinazolin-4-amine (described in example 209; 100 mg, 245 μmol), [2-(2-{[tert-butyl(dimethyl)silyl]oxy}ethyl)phenyl]boronic acid (68.6 mg, 245 μmol), K₂SO₃ (135 mg, 980 μmol) and Pd(PPh₃)₄ (28.3 mg, 24.5 μmol) in dioxane (2.5 mL) and H₂O (500 μL) were stirred at 110° C. overnight. H₂O was added, the mixture extracted with DCM and the solvent removed in vacuo. Purification by preparative HPLC (basic conditions) gave the title compound as a white solid (60.4 mg, 53%). ¹H-NMR (400 MHz, DMSO-d₆): δ [ppm]=8.15 (d, 1H), 7.65 (s, 1H), 7.35-7.31 (m, 1H), 7.30-7.25 (m, 2H), 7.23-7.18 (m, 1H), 7.08 (dd, 1H), 7.05 (s, 1H), 6.99 (d, 1H), 5.96 (quin, 1H), 4.67 (t, 1H), 3.87 (s, 6H), 3.54 (td, 2H), 2.84 (t, 2H), 2.43 (s, 3H), 1.71 (d, 3H). LC-MS (method 7): m/z: [M+H]⁺=450, Rt=0.89 min.

### Example 181

### 2-[3-(5-{1-[(6,7-dimethoxy-2-methylquinazolin-4-yl)amino]ethyl}thiophen-2-yl)phenyl]ethanol

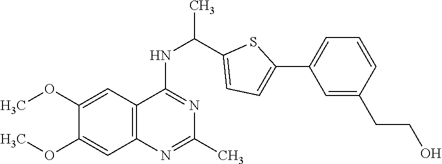 [SMILES: COc1cc2nc(C)nc(NC(C)c3ccc(-c4cccc(CCO)c4)s3)c2cc1OC]

Under argon, N-[1-(5-bromothiophen-2-yl)ethyl]-6,7-dimethoxy-2-methylquinazolin-4-amine (described in example 209; 100 mg, 245 μmol), [3-(2-hydroxyethyl)phenyl]boronic acid (40.7 mg, 245 μmol), K₂CO₃ (135 mg, 980 μmol) and Pd(PPh₃)₄ (28.3 mg, 24.5 μmol) in dioxane (2.5 mL) and H₂O (500 μL) were stirred at 110° C. overnight. H₂O was added, the mixture extracted with DCM and the solvent removed in vacuo. Purification by preparative HPLC (basic conditions) gave the title compound as a pale yellow solid (58.8 mg, 48%). ¹H-NMR (400 MHz, DMSO-d₆): δ [ppm]=8.13 (d, 1H), 7.65 (s, 1H), 7.43-7.38 (m, 2H), 7.33 (d, 1H), 7.26 (t, 1H), 7.11 (br d, 1H), 7.06 (dd, 1H), 7.05 (s, 1H), 5.93 (quin, 1H), 4.64 (t, 1H), 3.87 (s, 6H), 3.60 (td, 2H), 2.72 (t, 2H), 2.43 (s, 3H), 1.70 (d, 3H). LC-MS (method 7): m/z: [M+H]⁺=450, Rt=0.86 min.

### Example 182

### 6,7-di methoxy-2-methyl-N-[1-{5-[2-(2-oxa-6-azaspiro[3.3]hept-6-ylmethyl)phenyl]thiophen-2-yl}ethyl]quinazolin-4-amine

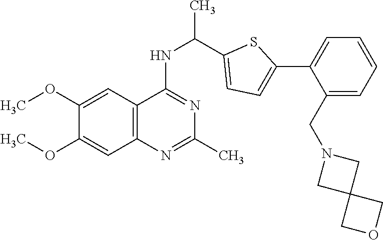 [SMILES: COc1cc2nc(C)nc(NC(C)c3ccc(-c4ccccc4CN4CC5(COC5)C4)s3)c2cc1OC]

To 2-(5-{1-[(6,7-dimethoxy-2-methylquinazolin-4-yl)amino]ethyl}thiophen-2-yl)benzaldehyde (prepared in example 179a; 50.0 mg, 115 μmol), 2-oxa-6-azaspiro[3.3]heptane (22.9 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as an off-white solid (39.7 mg, 65%). ¹H-NMR (400 MHz, DMSO-d₆): δ [ppm]=8.14 (d, 1H), 7.65 (s, 1H), 7.40-7.32 (m, 2H), 7.31-7.23 (m, 2H), 7.12 (d, 1H), 7.07 (dd, 1H), 7.05 (s, 1H), 5.96 (quin, 1H), 4.54 (s, 4H), 3.87 (s, 3H), 3.87 (s, 3H), 3.51 (s, 2H), 3.24 (s, 4H), 2.43 (s, 3H), 1.72 (d, 3H). LC-MS (method 7): m/z: [M+H]⁺=517, Rt=0.60 min.

### Example 183

### N-[1-(5-bromo-4-methylthiophen-2-yl)ethyl]-6,7-dimethoxy-2-methylquinazolin-4-amine

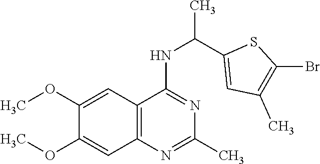 [SMILES: COc1cc2nc(C)nc(NC(C)c3cc(C)c(Br)s3)c2cc1OC]

A solution of 4-chloro-6,7-dimethoxy-2-methylquinazoline (commercially available; 789 mg, 3.30 mmol) and 1-(5-bromo-4-methylthiophen-2-yl)ethanamine (procedure described in INT-31; 800 mg, 3.63 mmol) in dioxane (15 mL) was heated to 110° C. during 18 hours. MTBE (20 mL) was added, the mixture stirred at room temperature during 3 hours then the precipitate filtered and dried to give the HCl salt of the title compound as an off-white solid (1.56 g, 90% purity, 93%). Purification of a sample by preparative HPLC (basic conditions) gave the title compound as yellow solid (29.1 mg, 2%). ¹H-NMR (400 MHz, DMSO-d₆): δ [ppm]=8.04 (d, 1H), 7.60 (s, 1H), 7.05 (s, 1H), 6.88 (s, 1H), 5.76 (quin, 1H), 3.87 (s, 6H), 2.42 (s, 3H), 2.09 (s, 3H), 1.64 (d, 3H). LC-MS (method 7): m/z: [M+H]⁺=422, Rt=0.97 min.

### Example 184

### 6,7-dimethoxy-2-methyl-N-[1-{5-[2-(pyrrolidin-1-ylmethyl)phenyl]thiophen-2-yl}ethyl]quinazolin-4-amine

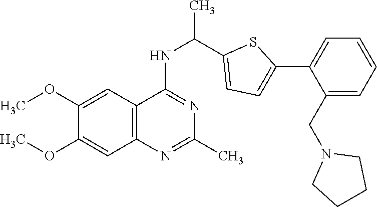 [SMILES: COc1cc2nc(C)nc(NC(C)c3ccc(-c4ccccc4CN4CCCC4)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), pyrrolidine (19 μL, 230 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as an off-white solid (39.8 mg, 71%). ¹H-NMR (400 MHz, DMSO-d₆): δ [ppm]=8.15 (d, 1H), 7.65 (s, 1H), 7.44-7.41 (m, 1H), 7.38-7.34 (m, 1H), 7.32-7.25 (m, 2H), 7.18 (d, 1H), 7.07 (dd, 1H), 7.05 (s, 1H), 5.97 (quin, 1H), 3.87 (s, 6H), 3.54 (s, 2H), 2.42 (s, 3H), 2.38 (br s, 4H), 1.72 (d, 3H), 1.60 (br s, 4H). LC-MS (method 7): m/z: [M+H]⁺=489, Rt=0.62 min.

### Example 185

### N-[1-{5-[2-({(2S)-[(dimethylamino)methyl]pyrrolidin-1-yl}methyl)phenyl]thiophen-2-yl}ethyl]-6,7-dimethoxy-2-methylquinazolin-4-amine

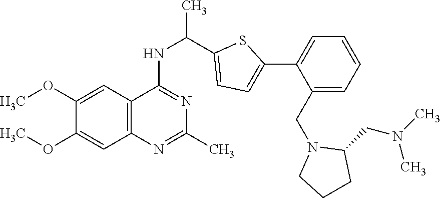

To 2-(5-{1-[(6,7-dimethoxy-2-methylquinazolin-4-yl)amino]ethyl}thiophen-2-yl)benzaldehyde (described in example 179a; 50.0 mg, 115 μmol), N,N-dimethyl-1-[(2S)-pyrrolidin-2-yl]methanamine (29.6 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a white solid (40.6 mg, 63%). ¹H-NMR (400 MHz, DMSO-d₆): δ [ppm]=8.13 (dd, 1H), 7.64 (s, 1H), 7.51-7.48 (m, 1H), 7.32-7.22 (m, 3H), 7.14 (dd, 1H), 7.08-7.06 (m, 1H), 7.05 (s, 1H), 5.96 (br quin, 1H), 4.07 (d, 1H), 3.87 (s, 6H), 3.36 (d, 1H), 2.77-2.70 (m, 1H), 2.42 (d, 3H), 2.08 (ddd, 1H), 2.05-2.01 (m, 1H), 2.00 (s, 3H), 1.99 (s, 3H), 1.88-1.75 (m, 1H), 1.72 (d, 3H), 1.58-1.43 (m, 3H). LC-MS (method 7): m/z: [M+H]⁺=546, Rt=0.64 min.

### Example 186

### 2-methyl-N-[(1R)-1-(naphthalen-1-yl)ethyl]quinazolin-4-amine

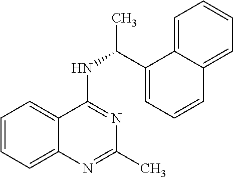 [SMILES: Cc1nc(N[C@H](C)c2cccc3ccccc23)c2ccccc2n1]

To a solution of 4-chloro-2-methylquinazoline (commercially available; 95 mg, 0.53 mmol) and (R)-(+)-1-(1-naphthyl)ethylamine (commercially available; 91 mg, 0.53 mmol) in DMSO (1 mL) was added TEA (108 mg, 1.1 mmol) was stirred at 50° C. for 16 h. The reaction mixture was filtered through a 0.45 μm Whatmann filter and purified by preparative HPLC (Autopurifier: basic conditions) to give the desired product (68 mg, 39%). ¹H-NMR (400 MHz, DMSO-d₆): δ [ppm]=1.71 (3H), 2.35 (3H), 6.42 (1H), 7.42 (1H), 7.46-7.62 (4H), 7.65-7.76 (2H), 7.82 (1H), 7.89-7.99 (1H), 8.31 (1H), 8.41 (1H), 8.46 (1H).

### Example 187

### N-[(1R)-1-(4-bromophenyl)ethyl]-6,7-dimethoxy-2-methylquinazolin-4-amine

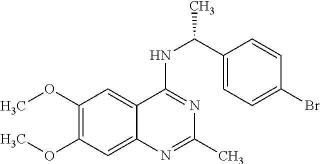 [SMILES: COc1cc2nc(C)nc(N[C@H](C)c3ccc(Br)cc3)c2cc1OC]

### Step a

### 4-chloro-6,7-dimethoxy-2-methylquinazoline

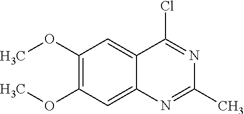

2.50 g (11.4 mmol) of 6,7-dimethoxy-2-methylquinazolin-4-ol were suspended in 11.00 mL of thionyl chloride, admixed with 10 drops of N,N-dimethylformamide and stirred for 1 h at 80° C. The thionyl chloride was removed in vacuo. The residue was admixed twice with in each case 20 mL of toluene and evaporated to dryness in vacuo. With ice bath cooling, 20 mL of a 5N sodium hydroxide solution were added dropwise to the residue. The suspension was admixed with 20 mL of dichloromethane and vigorously stirred for 10 min. The phases were separated and the aqueous phase was extracted by shaking five times with in each case 20 mL of dichloromethane. The combined organic phases were dried over sodium sulphate, filtered and evaporated to dryness in vacuo. 2.71 g (100% of theory) of the title compound were isolated as a beige solid. ¹H-NMR (400 MHz, CDCl₃): δ [ppm]=2.79 (s, 3H), 4.04 (s, 3H), 4.05 (s, 3H), 7.25 (s, 1H), 7.35 (s, 1H). LC-MS (method 1): Rₜ=3.19 min; MS (ESI/APCIpos) m/z=239.1 [M+H]⁺.

### Step b

### N-[(1R)-1-(4-bromophenyl)ethyl]-6,7-dimethoxy-2-methylquinazolin-4-amine

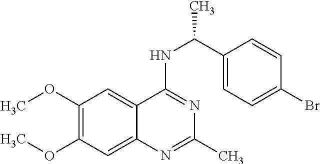 [SMILES: COc1cc2nc(C)nc(N[C@H](C)c3ccc(Br)cc3)c2cc1OC]

In the microwave vessel, under argon, 2.70 g (11.3 mmol) of 4-chloro-6,7-dimethoxy-2-methylquinazoline were dissolved in 15.0 mL of absolute 1,4-dioxane, admixed with 3.9 mL (22.6 mmol) of N,N-diisopropylethylamine and 2.72 g (13.6 mmol) of (R)-1-(4-bromophenyl)ethylamine (commercially available) and stirred for 16 h at 100° C. The reaction solution was evaporated to dryness in vacuo. The residue was taken off in 30 mL of dichloromethane and 30 mL of water. The phases were separated and the aqueous phase was extracted by shaking three times with in each case 25 mL of dichloromethane. The combined organic phases were dried over sodium sulphate, filtered and evaporated to dryness in vacuo. The residue (4 g) was chromatographed [silica gel 60 (2×80 g, 30 μm); dichloromethane/methanol (96:4)]. 2.91 g (64% of theory) of the title compound were isolated as a beige solid. ¹H-NMR (400 MHz, CDCl₃): δ [ppm]=1.63 (d, 3H), 2.55 (s, 3H), 3.91 (d, 6H), 5.64 (m, 1H), 5.78 (s, 1H), 6.96 (s, 1H), 7.11 (s, 1H), 7.28-7.32 (m, 2H), 7.91-7.43 (m, 2H). LC-MS (method 1): Rₜ=3.19 min; MS (ESI/APCIpos) m/z=402.1 [M+H]⁺.

### Example 188

### 6,7-dimethoxy-2-methyl-N-{(1R)-1-[4-(methylsulfonyl)phenyl]ethyl}quinazolin-4-amine

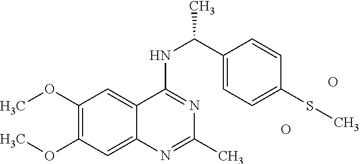

In the microwave vessel, 200 mg (0.50 mmol) of [(R)-1-(4-bromophenyl)ethyl](6,7-dimethoxy-2-methylquinazolin-4-yl)amine, 102 mg (0.99 mmol; prepared as described in example 187) of sodium sulphinate, 36 mg (99 μmol) of copper(II) trifluoromethanesulphonate and 23 mg (199 μmol) of (+−)-trans-1,2-diaminocyclohexane were dissolved in 3.0 mL of absolute dimethylsulphoxide and stirred for 21 h at 120° C. and for 7 h at 130° C. The reaction solution was evaporated to dryness in vacuo. The residue was admixed with 3 mL of water and extracted by shaking three times with in each case 5 mL of dichloromethane. The combined organic phases were dried over sodium sulphate, filtered and evaporated to dryness in vacuo. The residue was chromatographed [silica gel 60 (25 g, 30 μm); ethyl acetate/methanol (97:3)]. 122 mg (55% of theory) of the title compound were isolated as a beige solid. ¹H-NMR (400 MHz, CDCl₃): δ [ppm]=1.67 (d, 3H), 2.50 (s, 3H), 3.92 (s, 3H), 3.95 (s, 3H), 5.71 (m, 1H), 6.15 (s, 1H), 7.12 (m, 2H), 7.59 (d, 2H), 7.79 (s, 2H). LC-MS (method 1): Rₜ=2.72 min; MS (ESI/APCIpos) m/z=402.2 [M+H]⁺.

### Example 189

### 4-{(1R)-1-[(6,7-di methoxy-2-methylquinazolin-4-yl)amino]ethyl}benzonitrile

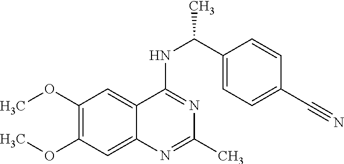 [SMILES: COc1cc2nc(C)nc(N[C@H](C)c3ccc(C#N)cc3)c2cc1OC]

In the microwave vessel, under argon, 300 mg (1.26 mmol) of 4-chloro-6,7-dimethoxy-2-methylquinazoline were dissolved in 3.00 mL of absolute 1,4-dioxane, admixed with 1.10 mL (6.29 mmol) of N,N-diisopropylethylamine and 344 mg (1.00 mmol) of (R)-4-(1-aminoethyl)benzonitrile hydrochloride and stirred for 16 h at 100° C. The batch was evaporated to dryness in vacuo. The residue was chromatographed three times [1^{st} column, silica gel 60 (40 g, 30 μm); dichloromethane/methanol (98:2 to 1:1); 2^{nd} column, silica gel 60 (25 g, 30 μm); dichloromethane/methanol (98:2 to 95:5); 3^{rd} column, silica gel 60 (25 g, 30 μm); ethyl acetate/methanol (95:5)]. 172 mg (39% of theory) of the title compound were isolated as a beige solid. ¹H-NMR (400 MHz, CDCl₃): δ [ppm]=1.67 (d, 3H), 2.50 (s, 3H), 3.94 (s, 3H), 3.96 (s, 3H), 5.67 (m, 1H), 5.78 (s, 1H), 6.98 (s, 1H), 7.13 (s, 1H), 7.52-7.58 (m, 4H). LC-MS (method 1): Rₜ=2.73 min; MS (ESI/APCIpos) m/z=402.2 [M+H]⁺.

### Example 190

### 6,7-dimethoxy-2-methyl-N-[(1R)-1-(3-methylphenyl)ethyl]quinazolin-4-amine

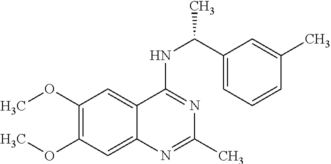

Under argon, a suspension of 100 mg (0.258 mmol) of [(R)-1-(3-bromophenyl)ethyl](6,7-dimethoxy-2-methylquinazolin-4-yl)amine (described in example 191) and 86 mg (0.386 mmol) of methylboronic acid in 1 mL of 1,2-dimethoxyethane and 0.3 mL of water was admixed with 18 mg (0.026 mmol) of bis(triphenylphosphine)palladium(II) dichloride and 107 mg (0.773 mmol) of potassium carbonate. The reaction mixture was degassed three times and stirred for 4 h at 90° C. The solution was admixed with 5 mL of water. The aqueous phase was extracted four times with in each case 20 mL of dichloromethane. The combined organic phases were dried over sodium sulphate and then evaporated to dryness. The residue was chromatographed on the Flashmaster [silica gel 60 (25 g, 50 μm); dichloromethane/methanol (98:2)]. 55 mg (30% of theory) of the title compound were isolated as a colourless solid. ¹H-NMR (400 MHz, DMSO): δ [ppm]=1.65-1.68 (m, 3H), 2.35 (s, 3H), 2.59 (s, 3H), 3.91-3.94 (m, 6H), 5.61-5.72 (m, 2H), 6.88 (s, 1H), 7.08-7.10 (m, 1H), 7.14 (s, 1H), 7.23-7.27 (m, 3H). LC-MS (method 1): m/z: [M+H]⁺=338.3, Rₜ=3.14 min.

### Example 191

### N-[(1R)-1-(3-bromophenyl)ethyl]-6,7-dimethoxy-2-methylquinazolin-4-amine

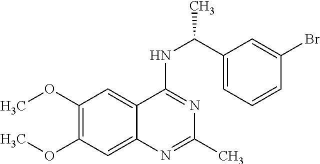 [SMILES: COc1cc2nc(C)nc(N[C@H](C)c3cccc(Br)c3)c2cc1OC]

### Step a

### 6,7-Dimethoxy-2-methylquinazolin-4-ol

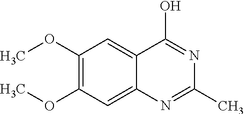

1.25 g (6.34 mmol) of 2-amino-4,5-dimethoxybenzoic acid, 1.2 g (12.68 mmol) of acetamidine hydrochloride and 1.04 g (12.678 mmol) of sodium acetate anhydrous were suspended in 20 mL of 2-methoxyethanol and stirred for 6 h under reflux. LCMS reveals complete conversion. After cooling to room temperature, the batch was admixed with 50 mL of water. The solid that has precipitated out in the process was filtered off with suction, washed three times with in each case 10 mL of cold water and evaporated to dryness in vacuo. 768 mg (55% of theory) of 6,7-dimethoxy-2-methylquinazolin-4-ol were isolated as a colourless solid. LC-MS (method 1): m/z: [M+H]⁺=221.2, Rₜ=2.28 min.

### Step b

### 4-Chloro-6,7-dimethoxy-2-methylquinazoline

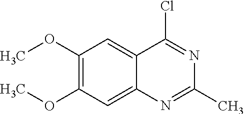

2.5 g (11.35 mmol) of 6,7-dimethoxy-2-methylquinazolin-4-ol were suspended in the Teflon vessel in 10 mL (137.1 mmol) of thionyl chloride and stirred with 10 drops of N,N-dimethylformamide and for 1 h at 85° C. The progress of the reaction was controlled by means of LC-MS. Complete conversion was detected. The reaction solution was evaporated to dryness in vacuo. The residue was taken up five times in 20 mL of toluene and evaporated to dryness in vacuo. Under ice cooling, 5 N sodium hydroxide solution was added until pH 8 was reached. Then, the aqueous phase was extracted four times with in each case 30 mL of chloroform. The combined organic phases were dried over sodium sulphate, filtered and evaporated to dryness in vacuo. 2.5 g (95% of theory) of 4-chloro-6,7-dimethoxy-2-methylquinazoline were isolated as a colourless solid. LC-MS (method 1): m/z: [M+H]⁺=239.1, Rₜ=3.17 min.

### Step c:

### N-[(1R)-1-(3-bromophenyl)ethyl]-6,7-dimethoxy-2-methylquinazolin-4-amine

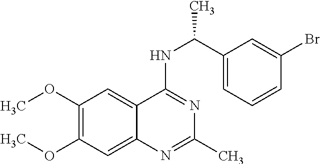 [SMILES: COc1cc2nc(C)nc(N[C@H](C)c3cccc(Br)c3)c2cc1OC]

2.57 g (10.77 mmol) of 4-chloro-6,7-dimethoxy-2-methylquinazoline were dissolved in 30 mL of 1,4-dioxane, admixed with 3.23 g (16.15 mmol) of (R)-3-bromo-alpha-methylbenzylamine (commercially available) and 3.75 mL (21.54 mmol) of Hunig's base and stirred for 18 h at 100° C. The progress of the reaction was monitored by means of LC-MS. Still no complete conversion was detected. A further 3.23 g (16.15 mmol) of (R)-3-bromo-alpha-methylbenzylamine and 3.75 mL (21.54 mmol) of Hunig's base were added and the mixture was stirred for 18 h at 100° C. The solvent was removed in vacuo. The residue was taken up in 50 mL of dichloromethane and washed with 20 mL of saturated sodium hydrogencarbonate solution and 20 mL of 1.0 N hydrochloric acid. The organic phase was evaporated to dryness in vacuo. The residue was chromatographed on the Flashmaster [silica gel 60 (120 g, 30 μm); dichloromethane/methanol (98:2)]. 2.79 g (64% of theory) of the title compound were isolated as a colourless solid. ¹H-NMR (400 MHz, CDCl₃): δ [ppm]=1.61-1.63 (m, 3H), 2.55 (s, 3H), 3.89-3.90 (m, 6H), 5.63-5.70 (m, 1H), 5.86-5.88 (m, 1H), 7.00 (s, 1H), 7.11 (s, 1H), 7.14-7.18 (m, 1H), 7.34-7.37 (m, 2H), 7.55-7.56 (m, 1H). LC-MS (method 1): m/z: [M+H]⁺=403.1, Rₜ=3.19 min.

### Example 192

### 4-{(1R)-1-[(6,7-dimethoxy-2-methylquinazolin-4-yl)amino]ethyl}benzamide

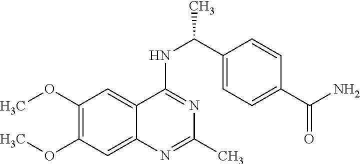 [SMILES: COc1cc2nc(C)nc(N[C@H](C)c3ccc(C(N)=O)cc3)c2cc1OC]

In the microwave vessel, 137 mg (0.39 mol) of 4-[(R)-1-(6,7-dimethoxy-2-methylquinazolin-4-ylamino)ethyl]benzonitrile (described in example 189) and 3 mg of hydrido(dimethyl-phosphinous acid-kP)[hydrogen bis(dimethylphosphinito-kP)]platinum(II) were suspended in 2.00 mL of ethanol and 2.00 mL of water and stirred for 5 h at 100° C. The reaction solution was evaporated to dryness in vacuo. The residue was chromatographed twice [1^{st} column silica gel 60 (25 g, 30 μm); ethyl acetate/methanol (95:5); 2^{nd} column silica gel 60 (25 g, 30 μm); dichloromethane/methanol (85:15)]. 73 mg (51% of theory) of the title compound were isolated as a colourless solid. ¹H-NMR (400 MHz, DMSO-d₆): δ [ppm]=1.59 (d, 3H), 2.33 (s, 3H), 3.86 (s, 3H), 3.1 (s, 3H), 5.66 (m, 1H), 7.02 (s, 1H), 7.25 (s, 1H), 7.50 (d, 2H), 7.71 (s, 1H), 7.80-7.85 (m, 2H), 7.86 (s, 1H), 7.99 (d, 1H). LC-MS (method 1): Rₜ=2.68 min; MS (ESI/APCIpos) m/z=367.2 [M+H]⁺.

### Example 193

### N-[(1R)-1-(biphenyl-3-yl)ethyl]-6,7-dimethoxy-2-methylquinazolin-4-amine

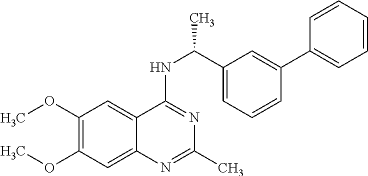 [SMILES: COc1cc2nc(C)nc(N[C@H](C)c3cccc(-c4ccccc4)c3)c2cc1OC]

Under argon, a suspension of 200 mg (0.497 mmol) of [(R)-1-(3-bromophenyl)ethyl](6,7-dimethoxy-2-methylquinazolin-4-yl)amine (described in example 191) and 152 mg (0.746 mmol) of phenylboronic acid pinacol ester in 2 mL of 1,2-dimethoxyethane and 0.6 mL of water was admixed with 35 mg (0.05 mmol) of bis(triphenylphosphine)palladium(II) dichloride and 275 mg (1.99 mmol) of potassium carbonate. The reaction mixture was degassed three times and stirred for 6 h at 90° C. The solution was admixed with 5 mL of water. The aqueous phase was extracted four times with in each case 20 mL of dichloromethane. The combined organic phases were dried over sodium sulphate and then evaporated to dryness. The residue was chromatographed twice on the Flashmaster [silica gel 60 (2×25 g, 50 μm); dichloromethane/methanol (98:2)]. 39 mg (19% of theory) of the title compound were isolated as a colourless solid. ¹H-NMR (400 MHz, DMSO): δ [ppm]=1.72-1.74 (m, 3H), 2.60 (s, 3H), 3.92-3.94 (m, 6H), 5.65-5.67 (m, 1H), 5.77-5.80 (m, 1H), 6.88 (s, 1H), 7.14 (s, 1H), 7.32-7.36 (m, 1H), 7.40-7.47 (m, 3H), 7.49-7.52 (m, 1H), 7.55-7.56 (m, 2H), 7.70 (s, 1H). LC-MS (method 1): m/z: [M+H]⁺=400.3, Rₜ=3.35 min.

### Example 194

### 3-{(1R)-1-[(6,7-dimethoxy-2-methylquinazolin-4-yl)amino]ethyl}benzonitrile

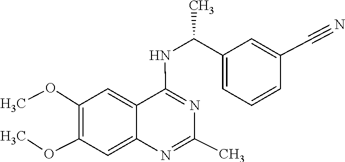 [SMILES: COc1cc2nc(C)nc(N[C@H](C)c3cccc(C#N)c3)c2cc1OC]

Under argon, a suspension of 400 mg (0.99 mmol) of [(R)-1-(3-bromophenyl)ethyl](6,7-dimethoxy-2-methylquinazolin-4-yl)amine (described in example 191) and 117 mg, (0.99 mmol) of zinc cyanide in 2.5 mL of absolute dimethylformamide was admixed with 81 mg (0.09 mmol) of [1,1′-bis(diphenylphosphino)ferrocene]dichloropalladium(II) complex in CH2Cl₂. The reaction mixture was stirred for 5 h at 100° C. For the work-up, the batch was admixed with a mixture of 10 mL of water and 10 mL of 25 percent ammonia. The aqueous phase was extracted three times using in each case 30 mL of dichloromethane. The combined organic phases were dried over sodium sulphate and then evaporated to dryness. The residue was chromatographed five times on the Flashmaster [silica gel 60 (5×25 g, 30 μm); chloroform/methanol (98:2)]. 70 mg (19% of theory) of the title compound were isolated as a colourless solid. ¹H-NMR (400 MHz, DMSO): δ [ppm]=1.64-1.65 (m, 3H), 2.51 (s, 3H), 3.91 3.94 (m, 6H), 5.63-5.70 (m, 1H), 5.98-5.99 (m, 1H), 7.06 (s, 1H), 7.10 (s, 1H), 7.38-7.42 (m, 1H), 7.47-7.50 (m, 1H), 7.60-7.71 (m, 2H). LC-MS (method 1): m/z: [M+H]⁺=349.2, Rₜ=2.87 min.

### Example 195

### 6,7-dimethoxy-2-methyl-N-[(1R)-1-(4-methylphenyl)ethyl]quinazolin-4-amine

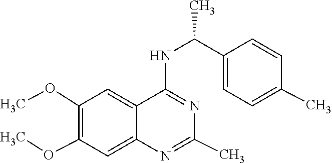

In the microwave vessel under argon, 200 mg (0.50 mmol) of [(R)-1-(4-bromophenyl)ethyl](6,7-dimethoxy-2-methylquinazolin-4-yl)amine (described in example 187), 89 mg (1.49 mmol) of methylboronic acid, 36 mg (0.05 mmol) of [bis(diphenylphosphino)ferrocene]-dichloropalladium(II) and 486 mg (1.49 mmol) of caesium carbonate were dissolved in 10.0 mL of absolute 1,4-dioxane, degassed three times, aerated with argon and stirred for 20 min at 100° C. The reaction solution was evaporated to dryness in vacuo. The residue was taken up in 50 mL of dichloromethane and washed three times with in each case 10 mL of water. The organic phase was dried over sodium sulphate and evaporated to dryness in vacuo. The residue was chromatographed [silica gel 60 (40 g, 30 μm); dichloromethane/methanol (96:4)]. 95 mg (54% of theory) of the title compound were isolated as an orange solid. ¹H-NMR (400 MHz, CDCl₃): δ [ppm]=1.66 (d, 3H), 2.34 (s, 3H), 2.59 (s, 3H), 3.93 (d, 6H), 5.68 (s, 1H), 5.68 (m, 1H), 6.86 (s, 1H), 7.15-7.17 (m, 3H), 7.35-7.37 (m, 2H). LC-MS (method 1): Rₜ=3.16 min; MS (ESI/APCIpos) m/z=338.3 [M+H]⁺.

### Example 196

### N-[(1R)-1-(biphenyl-4-yl)ethyl]-6,7-dimethoxy-2-methylquinazolin-4-amine

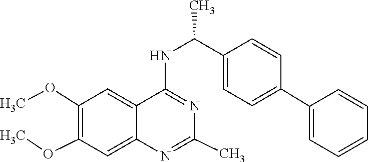 [SMILES: COc1cc2nc(C)nc(N[C@H](C)c3ccc(-c4ccccc4)cc3)c2cc1OC]

In the microwave vessel under argon, 250 mg (0.62 mmol) of [(R)-1-(4-bromophenyl)ethyl](6,7-dimethoxy-2-methylquinazolin-4-yl)amine (described in example 187), 190 mg (0.93 mmol) of phenylboronic acid pinacol ester, 44 mg (0.062 mmol) of bis(triphenylphosphine)palladium(II) chloride and 264 mg (1.24 mmol) of potassium phosphate were dissolved in 12 mL of absolute dimethoxyethane and 1.20 mL of water and stirred for 30 min at 90° C. The reaction solution was evaporated to dryness in vacuo. The residue was admixed with 30 mL of water and extracted by shaking five times with in each case 20 mL of dichloromethane. The combined organic phases were dried over sodium sulphate, filtered and evaporated to dryness in vacuo. The residue was chromatographed [silica gel 60 (40 g, 30 μm); dichloromethane/methanol (95:5)]. 98 mg (38% of theory) of the title compound were isolated as a colourless solid. ¹H-NMR (400 MHz, CDCl₃): δ [ppm]=1.73 (d, 3H), 2.61 (s, 3H), 3.97 (s, 6H), 5.61 (s br, 1H), 5.76 (m, 1H), 6.89 (m, 1H), 7.20 (m, 1H); 7.32-7.36 (m, 1H), 7.41-7.45 (m, 2H), 7.53-7.60 (m, 6H). LC-MS (method 1): Rₜ=3.44 min; MS (ESI/APCIpos) m/z=400.3 [M+H]⁺.

### Example 197

### N-[(1R)-1-(4-cyclopropylphenyl)ethyl]-6,7-dimethoxy-2-methylquinazolin-4-amine

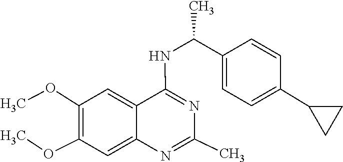 [SMILES: COc1cc2nc(C)nc(N[C@H](C)c3ccc(C4CC4)cc3)c2cc1OC]

In the microwave vessel under argon, 200 mg (0.50 mmol) of [(R)-1-(4-bromophenyl)-ethyl](6,7-dimethoxy-2-methylquinazolin-4-yl)amine (described in example 187), 128 mg (1.49 mmol) of cyclopropyl boronic acid, 36 mg (0.05 mmol) of [bis(diphenylphosphino)ferrocene]dichloro-palladium(II) and 485 mg (1.49 mmol) caesium carbonate were dissolved in 10.0 mL of absolute 1,4-dioxane, degassed three times, aerated with argon and stirred for 30 min at 100° C. The reaction solution was evaporated to dryness in vacuo. The residue was taken up in 50 mL of dichloromethane and washed three times with in each case 10 mL of water. The organic phase was dried over sodium sulphate and evaporated to dryness in vacuo. The residue was chromatographed [silica gel 60 (40 g, 30 μm); dichloromethane/methanol (96:4)]. 116 mg (61% of theory) of the title compound were isolated as an orange solid. ¹H-NMR (400 MHz, CDCl₃): δ [ppm]=0.66-0.70 (m, 2H), 0.93-0.97 (m, 2H), 1.66 (d, 3H), 1.88 (m, 1H), 2.60 (s, 3H), 3.94 (d, 6H), 5.57 (s, 1H), 5.67 (m, 1H), 6.83 (s, 1H), 7.05-7.07 (m, 2H), 7.26 (s, 1H), 7.26-7.37 (m, 2H). LC-MS (method 1): Rₜ=3.31 min; MS (ESI/APCIpos) m/z=364.3 [M+H]⁺.

### Example 198

### 6,7-dimethoxy-2-methyl-N-{(1R)-1-[3-(methylsulfonyl)phenyl]ethyl}quinazolin-4-amine

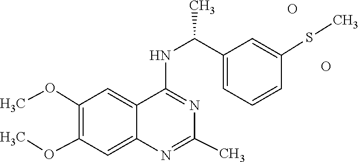

A solution of 200 mg (0.497 mmol) of [(R)-1-(3-bromophenyl)ethyl](6,7-dimethoxy-2-methylquinazolin-4-yl)amine (described in example 191), 56 mg (0.547 mmol) of sodium methanesulphinate, 18 mg (0.05 mmol) of copper(II) trifluoromethanesulphonate and 12 mg (0.099 mmol) of (±)-trans-1,2-diaminocyclohexane in 3 mL of dimethylsulphoxide was stirred at 100° C. for 1 week. The crude batch was purified five times by column chromatography on the Flashmaster [Puri-Flash, silica gel 60 (1×40 g, 4×25 g, 30 μm), chloroform/methanol 98:2]. 47 mg (21% of theory) of the title compound were isolated as colourless solid. ¹H-NM R (400 MHz, CDCl₃): δ [ppm]=1.63-1.65 (m, 3H), 2.5 (s, 3H), 3.01-3.93 (m, 3H), 5.68-5.71 (m, 1H), 6.04-6.06 (m, 1H), 7.05 (s, 3H), 7.11 (s, 1H), 7.47-7.51 (m, 1H), 7.74-7.79 (m, 2H), 8.03 8.04 (m, 1H). LC-MS (method 1): m/z: [M+H]⁺=402.2, Rₜ=2.73 min.

### Example 199

### 3-{(1R)-1-[(6,7-dimethoxy-2-methylquinazolin-4-yl)amino]ethyl}benzamide

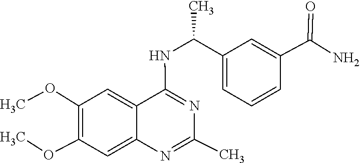 [SMILES: COc1cc2nc(C)nc(N[C@H](C)c3cccc(C(N)=O)c3)c2cc1OC]

In the microwave vessel, 114 mg (0.327 mmol) of 3-[(R)-1-(6,7-dimethoxy-2-methylquinazolin-4-ylamino)ethyl]benzonitrile (described in example 194) and 3 mg (5.76 μmol) of hydrido(dimethylphosphinous acid-kP)[hydrogenbis(dimethylphosphinito-kP)]platinum(II) were suspended in 2 mL of ethanol and 2 mL of water and stirred for 5 hat 100° C. The progress of the reaction was monitored by means of LC-MS. The reaction solution was brought to room temperature. The ethanol was removed in vacuo. During this, a solid precipitated out. The aqueous phase was extracted by shaking five times with in each case 2 mL of a mixture of dichloromethane:2-propanol (4:1). The combined organic phases were dried over sodium sulphate, filtered and evaporated to dryness in vacuo. The residue was chromatographed on the Flashmaster [silica gel 60 (40 g, 30 μm); dichloromethane/methanol (9:1)]. 79 mg (59% of theory) of the title compound were isolated as a colourless solid. ¹H-NMR (400 MHz, DMSO): δ [ppm]=1.56-1.58 (m, 3H), 2.5 (s, 3H), 3.78 (s, 3H), 3.84 (s, 3H), 5.66-5.73 (m, 1H), 6.15 (s, 1H), 6.51-6.57 (m, 1H), 7.00 (s, 1H), 7.12 (s, 1H), 7.27-7.31 (m, 2H), 7.55-7.59 (m, 2H), 7.94 (s, 1H). LC-MS (method 1): m/z: [M+H]⁺=367.2, Rₜ=2.72 min.

### Example 200

### 6,7-dimethoxy-2-methyl-N-{(1R)-1-[3-(1H-pyrazol-4-yl)phenyl]ethyl}quinazolin-4-amine

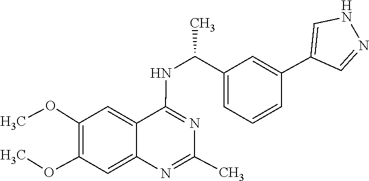 [SMILES: COc1cc2nc(C)nc(N[C@H](C)c3cccc(-c4cn[nH]c4)c3)c2cc1OC]

Under argon a suspension of 2.0 M sodiumcaronate solution (644 mL, 1.288 mmol), [(R)-1-(3-Bromphenyl)ethyl]-(6,7-dimethoxychinazolin-4-ylamine (described in example 191; 200 mg, 0.515 mmol), 4-pyrazoleboronic acid pinacolester (200 mg, 1.03 mmol) and Tetrakis(triphenyl-phosphine)palladium(0) (60 mg, 0.052 mmol) in DMF (2 mL) was stirred at 130° C. for 2 hours.

Water (5 mL) was added to the reaction and the aqueous layer was extracted with dichloromethane:isopropanol 4:1 (4×40 mL). The combined organic layers were dried over sodium sulfate, evaporated and the crude product was purified via Flashmaster column chromatography (silica gel 60, 30 μm, eluent: dichloromethane: methanol 98:2) to obtain the title compound in 8% yield (15 mg). ¹H-NMR (400 MHz, DMSO): δ [ppm]=1.61-1.63 (m, 3H), 3.89 (s, 3H), 3.94 (s, 3H), 5.62-5.65 (m, 1H), 7.08 (s, 1H), 7.25-7.30 (m, 2H), 7.43-7.46 (m, 1H), 7.66 (s, 1H), 7.77 (s, 1H), 8.06-8.07 (m, 1H), 8.28 (s, 1H). LC-MS (method 1): m/z: [M+H]⁺=376.3, Rₜ=2.78 min.

### Example 201

### 6,7-dimethoxy-2-methyl-N-{(1R)-1-[4-(1-methyl-1,2,3,6-tetrahydropyridin-4-yl)phenyl]ethyl}quinazolin-4-amine

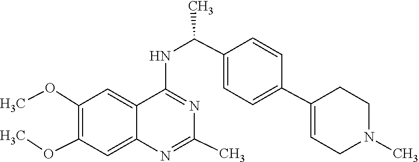

In the microwave vessel under argon, 200 mg (0.5 mmol) of [(R)-1-(4-bromophenyl)-ethyl](6,7-dimethoxy-2-methylquinazolin-4-yl)amine (described in example 187), 221 mg (0.99 mmol) of 1-methyl-1,2,3,6-tetrahydropyridine-4-boronic acid pinacol ester, 36 mg (0.05 mmol) of [bis(diphenylphosphino)ferrocene]dichloropalladium(II) and 486 mg (1.49 mmol) of caesium carbonate were dissolved in 10.0 mL of absolute 1,4-dioxane, degassed three times, aerated with argon and stirred for 1 h at 120° C. The reaction solution was evaporated to dryness in vacuo. The residue was taken up in 50 mL of dichloromethane and washed three times with in each case 10 mL of water. The organic phase was dried over sodium sulphate and evaporated to dryness in vacuo. The residue was chromatographed [silica gel 60 (40 g, 30 μm); dichloromethane/methanol (90:5 to 1:1)]. 102 mg (49% of theory) of the title compound were isolated as a brown solid. ¹H-NMR (400 MHz, CDCl₃): δ [ppm]=1.69 (d, 2H), 2.43 (s, 3H), 2.56-2.59 (m, 5H), 2.70 (m, 2H), 3.14 (s, 2H), 3.92 (d, 6H), 5.18 (m, 1H), 5.90-6.10 (m, 2H), 7.00 (s, 1H), 7.15 (s, 1H), 7.36 (d, 2H), 7.41 (d, 2H). LC-MS (method 1): Rₜ=2.47 min; MS (ESI/APCIpos) m/z=419.3 [M+H]⁺.

### Example 202

### 6,7-dimethoxy-2-methyl-N-{(1R)-1-[3-(1-methyl-1,2,3,6-tetrahydropyridin-4-yl)phenyl]ethyl}quinazolin-4-amine

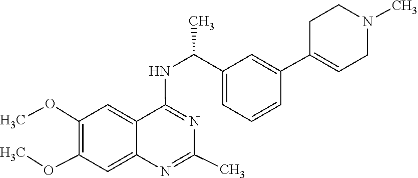

Under argon a suspension of [(R)-1-(3-bromphenyl)ethyl]-(6,7-dimethoxy-2-methylquinazolin-4-yl)amine (described in example 191; 300 mg, 0.746 mmol), 1-methyl-1,2,3,6-tetrahydropyridine-4-boronic acid pinacol ester (250 mg, 1.119 mmol), 1,2-dimethoxyethane (3 mL), water (0.9 mL), bis(triphenylphosphine)palladium(II)dichloride (53 mg, 0.075 mmol) and potassium carbonate (310 mg, 2.237 mmol) was prepared. The reaction mixture was degassed three times and stirred at 90° C. for 4 h. The course of the reaction was monitored by LC/MS. Mainly product was observed. The residue was dissolved in water (5 mL) and dichloromethane (10 mL) and the solution was extracted three times with 20 mL of dichlormethane. The combined organic layers were dried over sodium sulfate and then concentrated in vacuo. The crude product was purified by flash chromatography [silica gel 60 (25 g, 30 μm); chloroform/methanol 95:5]. 101 mg (32% d. Th.) of the title compound was isolated in form of a solid. ¹H-NMR (400 MHz, CDCl₃): δ [ppm]=1.67-1.69 (m, 3H), 2.39 (s, 3H), 2.58 (s, 5H), 2.64-2.67 (m, 2H), 3.08-3.11 (m, 2H), 3.91-3.93 (m, 6H), 5.61-5.71 (m, 2H), 6.03-6.04 (m, 1H), 6.88 (s, 1H), 7.12 (s, 1H), 7.28-7.36 (m, 3H), 7.50 (s, 1H). LC-MS (method 1): m/z: [M+H]⁺=419.3, Rₜ=2.46 min.

### Example 203

### 6,7-dimethoxy-2-methyl-N-{(1R)-1-[4-(prop-1-en-2-yl)phenyl]ethyl}quinazolin-4-amine

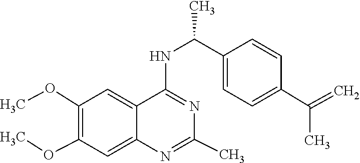 [SMILES: C=C(C)c1ccc([C@@H](C)Nc2nc(C)nc3cc(OC)c(OC)cc23)cc1]

A microwave vial was charged with 200 mg (0.5 mmol)N-[(1R)-1-(4-bromophenyl)ethyl]-6,7-dimethoxy-2-methylquinazolin-4-amine (described in example 187; 186 mg, 0.99 mmol) 2-isopropenyl-4,4,5,5-tetramethyl-1,3,2-dioxaborolane, 41 mg (0.05 mmol) [1,1′-bis(diphenylphosphino)ferrocene]dichloropalladium and 486 mg (1.50 mmol) caesium carbonate. The vial was sealed with a Teflon cap and the mixture was dissolved in 10.0 mL of dry 1,4-dioxane. The vial was degassed three times, refilled with argon, and the mixture was stirred at 100° C. for 2 h. The course of the reaction was monitored by LC/MS. Mainly product was observed. The mixture was concentrated in vacuo. The residue was dissolved in 100 mL of dichloromethane and the solution was washed three times with 50 mL of water. The combined organic layers were dried over sodium sulfate and then concentrated in vacuo. The crude product was purified by flash chromatography [silica gel 60, first column (40 g, 30 μm); dichloromethane/methanol (95:5), second column (12 g, 30 μm); ethyl acetate (100%)]. 42 mg (21%) of the title compound were obtained in form of a white-coloured solid. LC-MS (method 1): m/z: [M+H]⁺=364.3, Rₜ=3.39 min.

### Example 204

### N-[(1R)-1-(3-cyclopropylphenyl)ethyl]-6,7-dimethoxy-2-methylquinazolin-4-amine

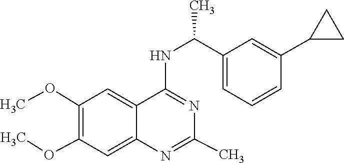 [SMILES: COc1cc2nc(C)nc(N[C@H](C)c3cccc(C4CC4)c3)c2cc1OC]

A microwave vial was charged with [(R)-1-(3-Bromphenyl)ethyl]-(6,7-dimethoxy-2-methylchinazolin-4-yl)amine (described in example 191; 396 mg, 0.984 mmol) and cyclopropylboronic acid pinacol ester (496 mg, 2.953 mmol), [1,1′-bis(diphenylphosphino)ferrocene]-dichloropalladium (72 mg, 0.098 mmol) and caesium carbonate (962 mg, 2.953 mmol). The vial was sealed with a teflon cap and the mixture was dissolved in dry 1,4-dioxane (3 mL). The vial was degassed three times, refilled with argon, and the mixture was stirred at 100° C. for 30 min. The course of the reaction was monitored by LC/MS. The mixture was concentrated in vacuo. The residue was dissolved in dichloromethane (50 mL) and the solution was washed with water (3×50 mL). The combined organic layers were dried over sodium sulfate and then concentrated in vacuo. The crude product was purified by flash chromatography [silica gel 60 (40 g, 30 μm); dichloromethane/methanol 95:5]. The thus obtained 95 mg of crude product was purified by preparative HPLC (column: Luna 5 μm phenyl-hexyl; acetonitrile/water/DEA; 250×30 mm; 65:35:0.1) to yield the title compound (13 mg, 3%) as yellow solid. ¹H-NMR (400 MHz, CDCl₃): δ [ppm]=0.68-0.72 (m, 2H), 0.94 0.99 (m, 2H), 1.67-1.69 (m, 3H), 1.87-1.92 (m, 1H), 2.61 (s, 3H), 3.95-3.96 (m, 6H), 5.50-5.68 (m, 2H), 6.84 (s, 1H), 6.95-6.98 (m, 1H), 7.18-7.25 (m, 4H). LC-MS (method 1): m/z: [M+H]⁺=364.2, Rₜ=3.81 min.

### Example 205

### N-[1-(1-benzothiophen-4-yl)ethyl]-6,7-dimethoxy-2-methylquinazolin-4-amine

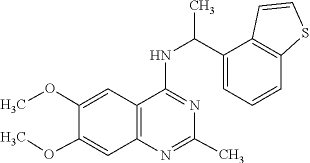 [SMILES: COc1cc2nc(C)nc(NC(C)c3cccc4sccc34)c2cc1OC]

### Step a

### 1-(1-benzothiophen-4-yl)ethanamine

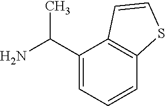

The title compound was prepared in analogy to 1-(5,6,7,8-tetrahydronaphthalen-1-yl)ethanamine (INT-1) from 4-bromo-1-benzothiophene (commercially available, 1.00 g, 4.69 mmol) and used directly in step b.

### Step b

### N-[1-(1-benzothiophen-4-yl)ethyl]-6,7-dimethoxy-2-methylquinazolin-4-amine

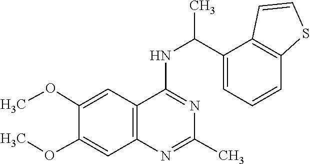 [SMILES: COc1cc2nc(C)nc(NC(C)c3cccc4sccc34)c2cc1OC]

To a microwave vial were added 4-chloro-6,7-dimethoxy-2-methylquinazoline (commercially available; 1.02 g, 4.27 mmol), 1-(1-benzothiophen-4-yl)ethanamine (832 mg, 4.69 mmol), N,N-diisopropylethylamine (1.9 mL, 11 mmol) and DMSO (17 mL). The reaction mixture was heated to 130° C. during 2.5 hours in the microwave. The mixture was diluted with H₂O, extracted with DCM and the solvent removed in vacuo. Purification by preparative HPLC (basic conditions) gave the title compound as an off-white solid (234 mg, 14%). ¹H-NMR (400 MHz, DMSO-d₆): δ [ppm]=8.10 (s, 1H), 7.90 (d, 1H), 7.78 (d, 1H), 7.77-7.75 (m, 1H), 7.71 (s, 1H), 7.51 (d, 1H), 7.37 (t, 1H), 7.02 (s, 1H), 6.18 (quin, 1H), 3.89 (s, 3H), 3.86 (s, 3H), 2.33 (s, 3H), 1.69 (d, 3H).

LC-MS (method 7): m/z: [M+H]⁺=380, Rₜ=1.25 min.

### Example 206

### 6,7-dimethoxy-2-methyl-N-[(1R)-1-phenylethyl]quinazolin-4-amine

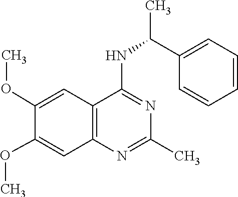 [SMILES: COc1cc2nc(C)nc(N[C@H](C)c3ccccc3)c2cc1OC]

To a microwave vial were added 4-chloro-6,7-dimethoxy-2-methylquinazoline (commercially available; 1.00 g, 4.19 mmol), (1R)-1-phenylethanamine (commercially available; 590 μL, 4.6 mmol), N,N-diisopropylethylamine (1.9 mL, 11 mmol) and DMSO (17 mL). The reaction mixture was heated to 130° C. during 2.5 hours in the microwave. The mixture was diluted with H₂O, extracted with DCM and the solvent removed in vacuo. The crude residue was purified by column chromatography (silica gel, hexane/EtOAc 50-100%). The resulting residue was stirred in Et₂O over the weekend then filtered to give the title compound as a pale yellow solid (909 mg, 66%). ¹H-NMR (400 MHz, DMSO-d₆): δ [ppm]=7.99 (d, 1H), 7.71 (s, 1H), 7.46 (s, 1H), 7.44 (s, 1H), 7.32 (t, 2H), 7.22 (tt, 1H), 7.02 (s, 1H), 5.67 (quin, 1H), 3.90 (s, 3H), 3.86 (s, 3H), 2.35 (s, 3H), 1.59 (d, 3H). LC-MS (method 7): m/z: [M+H]⁺=324, Rₜ=0.76 min.

### Example 207

### 6,7-dimethoxy-2-methyl-N-[1-(thiophen-2-yl)ethyl]quinazolin-4-amine

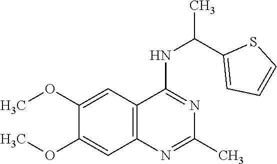 [SMILES: COc1cc2nc(C)nc(NC(C)c3cccs3)c2cc1OC]

### Step a

### 1-(thiophen-2-yl)ethanamine

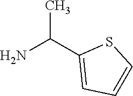

The title compound was prepared in analogy to 1-(5-bromothiophen-2-yl)ethanamine (INT-28) from thiophene-2-carbaldehyde (commercially available, 112 mg, 1.00 mmol) to give 24.3 mg of the title compound.

### Step b

### 6,7-dimethoxy-2-methyl-N-[1-(thiophen-2-yl)ethyl]quinazolin-4-amine

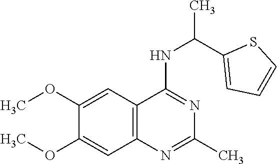 [SMILES: COc1cc2nc(C)nc(NC(C)c3cccs3)c2cc1OC]

To a microwave vial were added 4-chloro-6,7-dimethoxy-2-methylquinazoline (commercially available; 41.4 mg, 174 μmol), 1-(thiophen-2-yl)ethanamine (24.3 mg, 191 μmol), N,N-diisopropylethylamine (77 μL, 450 μmol) and DMSO (1.3 mL). The reaction mixture was heated to 130° C. during 2 hours in the microwave. Purification by preparative HPLC (basic conditions) gave the title compound as a white solid (17 mg, 30%). ¹H-NMR (400 MHz, DMSO-d₆): δ [ppm]=8.09 (d, 1H), 7.64 (s, 1H), 7.36 (dd, 1H), 7.08 (d, 1H), 7.05 (s, 1H), 6.98 (dd, 1H), 5.94 (quin, 1H), 3.87 (d, 6H), 2.42 (s, 3H), 1.69 (d, 3H). LC-MS (method 7): m/z: [M+H]⁺=330, Rₜ=0.72 min.

### Example 208

### N-[1-(5-bromofuran-2-yl)ethyl]-6,7-dimethoxy-2-methylquinazolin-4-amine

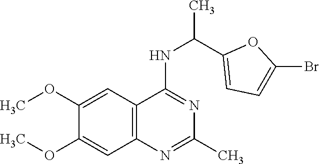 [SMILES: COc1cc2nc(C)nc(NC(C)c3ccc(Br)o3)c2cc1OC]

### Step a

### 1-(5-bromofuran-2-yl)ethanamine

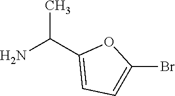

The title compound was prepared in analogy to 1-(5-bromothiophen-2-yl)ethanamine (INT-28) from 5-bromo-2-furaldehyde (commercially available, 3.00 g, 17.1 mmol) to give 1.67 g of the title compound.

### Step b

### N-[1-(5-bromofuran-2-yl)ethyl]-6,7-dimethoxy-2-methylquinazolin-4-amine

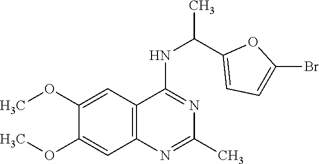 [SMILES: COc1cc2nc(C)nc(NC(C)c3ccc(Br)o3)c2cc1OC]

To a microwave vial were added 4-chloro-6,7-dimethoxy-2-methylquinazoline (commercially available; 343 mg, 1.44 mmol), 1-(5-bromofuran-2-yl)ethanamine (300 mg, 1.58 mmol), N,N-diisopropylethylamine (640 μL, 3.7 mmol) and DMSO (10 mL). The reaction mixture was heated to 130° C. during 2 hours in the microwave. Purification by preparative HPLC (basic conditions) gave the title compound as a beige solid (1.13 g, 45%). 1H-NMR (400 MHz, DMSO-d₆): δ [ppm]=8.01 (d, 1H), 7.63 (s, 1H), 7.05 (s, 1H), 6.51 (d, 1H), 6.41 (dd, 1H), 5.75 (quin, 1H), 3.87 (s, 6H), 2.42 (s, 3H), 1.55 (d, 3H). LC-MS (method 7): m/z: [M+H]⁺=392, Rₜ=0.85 min.

### Example 209

### N-[1-(5-bromothiophen-2-yl)ethyl]-6,7-dimethoxy-2-methylquinazolin-4-amine

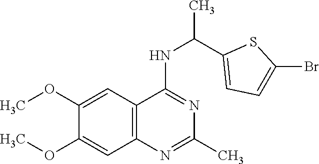 [SMILES: COc1cc2nc(C)nc(NC(C)c3ccc(Br)s3)c2cc1OC]

To a microwave vial were added 4-chloro-6,7-dimethoxy-2-methylquinazoline (commercially available; 2.39 g, 10.0 mmol), 1-(5-bromothiophen-2-yl)ethanamine (described in procedure INT-28; 2.27 g, 11.0 mmol), N,N-diisopropylethylamine (4.5 mL, 26 mmol) and DMSO (20 mL). The reaction mixture was heated to 130° C. during 6 hours in the microwave. The mixture was added to H₂O, extracted with DCM and the solvent removed in vacuo. The residue was stirred in Et₂O during 20 minutes then filtered to give the title compound as a beige solid (3.00 g, 73%). 1H-NMR (400 MHz, DMSO-d₆): δ [ppm]=8.09 (d, 1H), 7.60 (s, 1H), 7.06 (d, 1H), 7.05 (s, 1H), 6.92 (dd, 1H), 5.78 (quin, 1H), 3.87 (d, 6H), 2.43 (s, 3H), 1.66 (d, 3H). LC-MS (method 7): m/z: [M+H]⁺=408, Rₜ=0.91 min.

### Example 210

### 1-[2-(5-{1-[(6,7-dimethoxy-2-methylquinazolin-4-yl)amino]ethyl}thiophen-2-yl)benzyl]pyrrolidin-3-ol

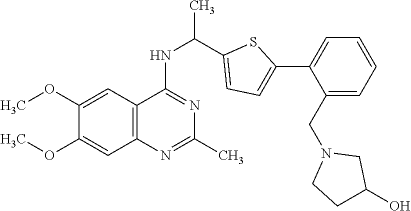 [SMILES: COc1cc2nc(C)nc(NC(C)c3ccc(-c4ccccc4CN4CCC(O)C4)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), pyrrolidin-3-ol (19 μL, 230 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a white solid (38.8 mg, 66%). ¹H-NM R (400 MHz, DMSO-d₆): δ [ppm]=8.15 (d, 1H), 7.65 (s, 1H), 7.44 (dd, 1H), 7.37-7.33 (m, 1H), 7.33-7.24 (m, 2H), 7.20 (d, 1H), 7.07 (dd, 1H), 7.05 (s, 1H), 5.97 (quin, 1H), 4.64 (dd, 1H), 4.18-4.09 (m, 1H), 3.87 (s, 6H), 3.55 (d, 1H), 3.51 (d, 1H), 2.66-2.62 (m, 1H), 2.57-2.53 (m, 1H), 2.43 (s, 3H), 2.40-2.35 (m, 1H), 2.30-2.24 (m, 1H), 1.95-1.84 (m, 1H), 1.71 (d, 3H), 1.52-1.43 (m, 1H). LC-MS (method 7): m/z: [M+H]⁺=505, Rt=0.59 min.

### Example 211

### N-{1-[5-(2-{[(3S)-3-fluoropyrrolidin-1-yl]methyl}phenyl)thiophen-2-yl]ethyl}-6,7-dimethoxy-2-methylquinazolin-4-amine

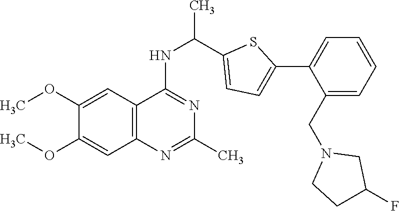 [SMILES: COc1cc2nc(C)nc(NC(C)c3ccc(-c4ccccc4CN4CCC(F)C4)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), 3-fluoropyrrolidine (20.6 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a white solid (31.6 mg, 54%). ¹H-NMR (400 MHz, DMSO-d₆): δ [ppm]=8.15 (d, 1H), 7.65 (s, 1H), 7.45 (dd, 1H), 7.38-7.35 (m, 1H), 7.34-7.27 (m, 2H), 7.18 (dd, 1H), 7.08 (dd, 1H), 7.05 (s, 1H), 5.97 (quin, 1H), 5.13 (br d, 1H), 3.87 (s, 3H), 3.87 (s, 3H), 3.59 (d, 1H), 3.56 (dd, 1H), 2.79-2.68 (m, 2H), 2.65-2.53 (m, 1H), 2.42 (s, 3H), 2.28 (br q, 1H), 2.13-1.95 (m, 1H), 1.88 1.74 (m, 1H), 1.72 (d, 3H). LC-MS (method 7): m/z: [M+H]⁺=507, Rt=0.62 min.

### Example 212

### 6,7-dimethoxy-2-methyl-N-[1-(quinolin-5-yl)ethyl]quinazolin-4-amine

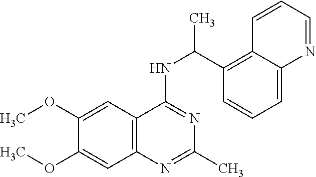 [SMILES: COc1cc2nc(C)nc(NC(C)c3cccc4ncccc34)c2cc1OC]

### Step a

### 1-(quinolin-5-yl)ethanamine

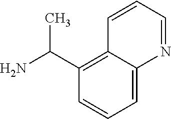

The title compound was prepared in analogy to 1-(5-bromothiophen-2-yl)ethanamine (INT-28) from quinoline-8-carbaldehyde (commercially available, 1.00 g, 6.36 mmol) to give 206 mg of the title compound.

### Step b

### 6,7-dimethoxy-2-methyl-N-[1-(quinolin-5-yl)ethyl]quinazolin-4-amine

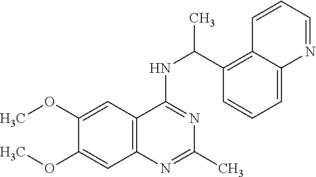 [SMILES: COc1cc2nc(C)nc(NC(C)c3cccc4ncccc34)c2cc1OC]

To a microwave vial were added 4-chloro-6,7-dimethoxy-2-methylquinazoline (commercially available; 50.0 mg, 209 μmol), 1-(quinolin-5-yl)ethanamine (39.7 mg, 230 μmol), N,N-diisopropylethylamine (93 μL, 540 μmol) and DMSO (1.5 mL). The reaction mixture was heated to 130° C. during 2 hours in the microwave. Purification by preparative HPLC (acidic conditions) gave the title compound as a white solid (14.6 mg, 19%). ¹H-NMR (400 MHz, DMSO-d₆): δ [ppm]=8.91 (dd, 1H), 8.77 (d, 1H), 8.19 (d, 1H), 7.94 (dd, 1H), 7.81-7.73 (m, 2H), 7.70 (s, 1H), 7.58 (dd, 1H), 7.03 (s, 1H), 6.40 (quin, 1H), 3.88 (s, 3H), 3.86 (s, 3H), 2.31 (s, 3H), 1.72 (d, 3H). LC-MS (method 7): m/z: [M+H]⁺=375, Rₜ=0.58 min.

### Example 213

### 6,7-dimethoxy-2-methyl-N-[1-(5-phenylfuran-2-yl)ethyl]quinazolin-4-amine

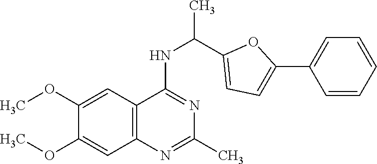 [SMILES: COc1cc2nc(C)nc(NC(C)c3ccc(-c4ccccc4)o3)c2cc1OC]

### Step a

### 1-(5-phenylfuran-2-yl)ethanamine

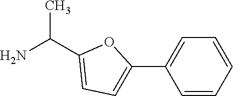

The title compound was prepared in analogy to 1-(5-bromothiophen-2-yl)ethanamine (INT-28) from 5-phenyl-2-furaldehyde (commercially available, 300 mg, 1.74 mmol) to give 25 mg of the title compound.

### Step b

### 6,7-dimethoxy-2-methyl-N-[1-(5-phenylfuran-2-yl)ethyl]quinazolin-4-amine

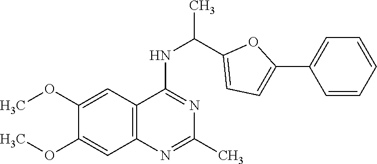 [SMILES: COc1cc2nc(C)nc(NC(C)c3ccc(-c4ccccc4)o3)c2cc1OC]

To a microwave vial were added 4-chloro-6,7-dimethoxy-2-methylquinazoline (commercially available; 29.0 mg, 121 μmol), 1-(5-phenylfuran-2-yl)ethanamine (25.0 mg, 134 μmol), N,N-diisopropylethylamine (54 μL, 320 μmol) and DMSO (900 μL). The reaction mixture was heated to 130° C. during 2 hours in the microwave. Purification by preparative HPLC (acidic conditions) gave the title compound as a light orange solid (9.4 mg, 20%). ¹H-NMR (500 MHz, DMSO-d₆): δ [ppm]=7.68 (s, 1H), 7.64 (d, 1H), 7.62 (d, 1H), 7.38 (t, 2H), 7.27-7.22 (m, 1H), 7.04 (s, 1H), 6.88 (d, 1H), 6.44 (d, 1H), 5.84 (quin, 1H), 3.87 (s, 3H), 3.86 (s, 3H), 2.45 (s, 3H), 1.63 (d, 3H). LC-MS (method 7): m/z: [M+H]⁺=390, Rₜ=0.95 min.

### Example 214

### N-[1-(5-bromo-2,3-dihydro-1-benzofuran-7-yl)ethyl]-6,7-dimethoxy-2-methylquinazolin-4-amine

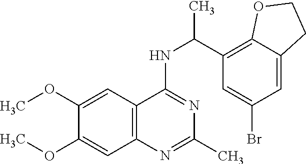 [SMILES: COc1cc2nc(C)nc(NC(C)c3cc(Br)cc4c3OCC4)c2cc1OC]

### Step a

### 1-(5-bromo-2,3-dihydro-1-benzofuran-7-yl)ethanamine

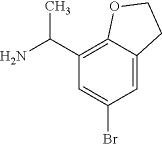

The title compound was prepared in analogy to 1-(5-bromothiophen-2-yl)ethanamine (INT-28) from 5-bromo-2,3-dihydro-1-benzofuran-7-carbaldehyde (commercially available, 1.00 g, 4.40 mmol) to give 65 mg of the title compound.

### Step b

### N-[1-(5-bromo-2,3-dihydro-1-benzofuran-7-yl)ethyl]-6,7-dimethoxy-2-methylquinazolin-4-amine

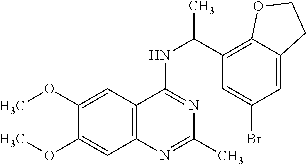 [SMILES: COc1cc2nc(C)nc(NC(C)c3cc(Br)cc4c3OCC4)c2cc1OC]

To a microwave vial were added 4-chloro-6,7-dimethoxy-2-methylquinazoline (commercially available; 64.1 mg, 268 μmol), 1-(5-bromo-2,3-dihydro-1-benzofuran-7-yl)ethanamine (65.0 mg, 268 μmol), N,N-diisopropylethylamine (120 μL, 700 μmol) and DMSO (2.0 mL). The reaction mixture was heated to 130° C. during 2 hours in the microwave. Purification by preparative HPLC (acidic conditions) gave the title compound as a yellow solid (51.9 mg, 44%). ¹H-NMR (500 MHz, DMSO-d₆): δ [ppm]=7.93 (d, 1H), 7.71 (s, 1H), 7.26 (s, 2H), 7.02 (s, 1H), 5.66 (quin, 1H), 4.65-4.55 (m, 2H), 3.91 (s, 3H), 3.86 (s, 3H), 3.20 (br t, 2H), 2.32 (s, 3H), 1.50 (d, 3H). LC-MS (method 7): m/z: [M+H]⁺=444, Rₜ=0.93 min.

### Example 215

### 6,7-dimethoxy-2-methyl-N-[1-(3-phenoxyphenyl)ethyl]quinazolin-4-amine

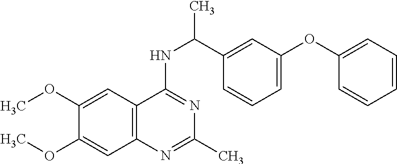 [SMILES: COc1cc2nc(C)nc(NC(C)c3cccc(Oc4ccccc4)c3)c2cc1OC]

### Step a

### 1-(3-phenoxyphenyl)ethanamine

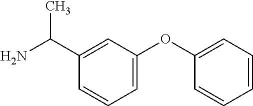 [SMILES: CC(N)c1cccc(Oc2ccccc2)c1]

The title compound was prepared in analogy to 1-(5-bromothiophen-2-yl)ethanamine (INT-28) from 3-phenoxybenzaldehyde (commercially available, 1.00 g, 5.05 mmol) to give 18 mg of the title compound.

### Step b

### 6,7-dimethoxy-2-methyl-N-[1-(3-phenoxyphenyl)ethyl]quinazolin-4-amine

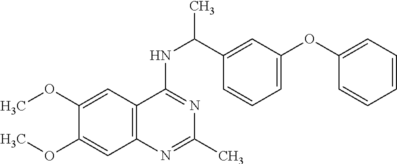 [SMILES: COc1cc2nc(C)nc(NC(C)c3cccc(Oc4ccccc4)c3)c2cc1OC]

To a microwave vial were added 4-chloro-6,7-dimethoxy-2-methylquinazoline (commercially available; 18.3 mg, 76.7 μmol), 1-(3-phenoxyphenyl)ethanamine (18.0 mg, 84.4 μmol), N,N-diisopropylethylamine (34 μL, 200 μmol) and DMSO (600 μL). The reaction mixture was heated 130° C. during 2 hours in the microwave. Purification by preparative HPLC (acidic conditions) gave the title compound as a light yellow solid (6.00 mg, 19%). ¹H-NMR (500 MHz, DMSO-d₆): δ [ppm]=7.98 (d, 1H), 7.67 (s, 1H), 7.38-7.32 (m, 3H), 7.23 (d, 1H), 7.14-7.10 (m, 2H), 7.03 (s, 1H), 6.99-6.98 (m, 1H), 6.98-6.96 (m, 1H), 6.83 (dd, 1H), 5.61 (quin, 1H), 3.89 (s, 3H), 3.87 (s, 3H), 2.33 (s, 3H), 1.58 (d, 3H). LC-MS (method 7): m/z: [M+H]⁺=416, Rₜ=0.99 min.

### Example 216

### 6,7-dimethoxy-2-methyl-N-{1-[3-(2H-tetrazol-5-yl)phenyl]ethyl}quinazolin-4-amine

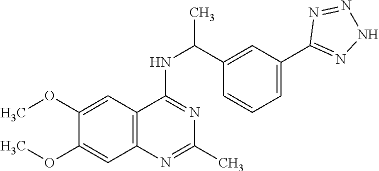 [SMILES: COc1cc2nc(C)nc(NC(C)c3cccc(-c4nn[nH]n4)c3)c2cc1OC]

### Step a

### 1-[3-(2H-tetrazol-5-yl)phenyl]ethanamine

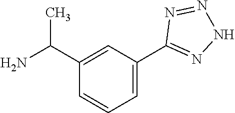

The title compound was prepared in analogy to 1-(5-bromothiophen-2-yl)ethanamine (INT-28) from 3-(2H-tetrazol-5-yl)benzaldehyde (commercially available, 1.00 g, 4.69 mmol) to give 165 mg of the title compound.

### Step b

### 6,7-dimethoxy-2-methyl-N-{1-[3-(2H-tetrazol-5-yl)phenyl]ethyl}quinazolin-4-amine

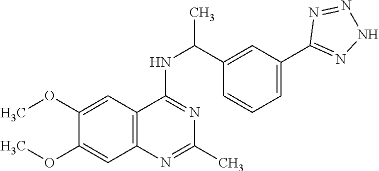 [SMILES: COc1cc2nc(C)nc(NC(C)c3cccc(-c4nn[nH]n4)c3)c2cc1OC]

To a microwave vial were added 4-chloro-6,7-dimethoxy-2-methylquinazoline (commercially available; 57.8 mg, 242 μmol), 1-[3-(2H-tetrazol-5-yl)phenyl]ethanamine (168 mg, 30% purity, 266 μmol), N,N-diisopropylethylamine (110 μL, 630 μmol) and DMSO (1.8 mL). The reaction mixture was heated to 130° C. during 2 hours in the microwave. Purification by preparative HPLC (basic conditions) gave the title compound as a light yellow solid (4.00 mg, 4%). ¹H-NMR (500 MHz, DMSO-d₆): δ [ppm]=8.48 (br d, 1H), 8.18-8.11 (m, 1H), 7.87 (dt, 1H), 7.79 (s, 1H), 7.50 (br d, 1H), 7.45 (t, 1H), 7.03 (s, 1H), 5.75 (quin, 1H), 3.92 (s, 3H), 3.88 (s, 3H), 2.41 (s, 3H), 1.66 (d, 3H). LC-MS (method 7): m/z: [M+H]⁺=392, Rₜ=0.64 min.

### Example 217

### 6,7-dimethoxy-2-methyl-N-[1-(quinolin-8-yl)ethyl]quinazolin-4-amine

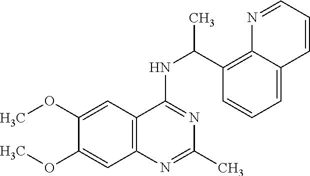 [SMILES: COc1cc2nc(C)nc(NC(C)c3cccc4cccnc34)c2cc1OC]

### Step a

### 1-(quinolin-8-yl)ethanamine hydrochloride (1:1)

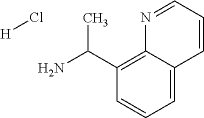

The title compound was prepared in analogy to 1-(5-bromothiophen-2-yl)ethanamine (INT-28) from quinoline-8-carbaldehyde (commercially available, 1.00 g, 4.69 mmol) to give 50 mg of the title compound.

### Step b

### 6,7-dimethoxy-2-methyl-N-[1-(quinolin-8-yl)ethyl]quinazolin-4-amine

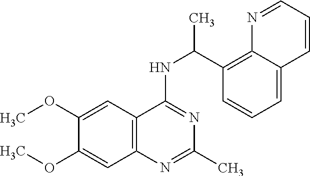 [SMILES: COc1cc2nc(C)nc(NC(C)c3cccc4cccnc34)c2cc1OC]

To a microwave vial were added 4-chloro-6,7-dimethoxy-2-methylquinazoline (commercially available; 52.0 mg, 218 μmol), 1-(quinolin-8-yl)ethanamine hydrochloride (1:1) (50.0 mg, 240 μmol), N,N-diisopropylethylamine (130 μL, 780 μmol) and DMSO (1.6 mL). The reaction mixture was heated to 130° C. during 2 hours in the microwave. Purification by preparative HPLC (acidic conditions) gave the title compound as a light yellow solid (4.3 mg, 5%). ¹H-NMR (600 MHz, DMSO-d₆): δ [ppm]=9.04 (dd, 1H), 8.40 (dd, 1H), 8.24 (br d, 1H), 7.88-7.85 (m, 1H), 7.83 (br s, 1H), 7.82 (s, 1H), 7.60 (dd, 1H), 7.57 (t, 1H), 7.03 (s, 1H), 6.75 (quin, 1H), 3.98 (s, 3H), 3.88 (s, 3H), 2.21 (s, 3H), 1.71 (d, 3H). LC-MS (method 8): m/z: [M+H]⁺=375, Rₜ=0.96 min.

### Example 218

### 2-[4-(5-{1-[(6,7-dimethoxy-2-methylquinazolin-4-yl)amino]ethyl}thiophen-2-yl)-1H-pyrazol-1-yl]ethanol

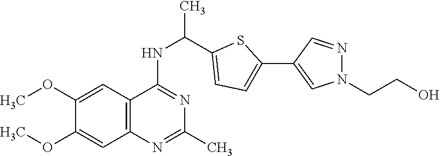 [SMILES: COc1cc2nc(C)nc(NC(C)c3ccc(-c4cnn(CCO)c4)s3)c2cc1OC]

Under argon, N-[1-(5-bromothiophen-2-yl)ethyl]-6,7-dimethoxy-2-methylquinazolin-4-amine (described in example 209; 50.0 mg, 122 μmol), 2-[4-(4,4,5,5-tetramethyl-1,3,2-dioxaborolan-2-yl)-1H-pyrazol-1-yl]ethanol (29.2 mg, 122 μmol), K₂CO₃ (67.7 mg, 490 μmol) and Pd(PPh₃)₄ (7.08 mg, 6.12 μmol) in dioxane (1.3 mL) and H₂O (250 μL) were stirred at 110° C. overnight.

Brine was added, the mixture extracted with DCM and the solvent removed in vacuo. Purification by preparative HPLC (basic conditions) gave the title compound as a light yellow solid (26.4 mg, 49%). ¹H-NM R (400 MHz, DMSO-d₆): δ [ppm]=8.53 (br s, 1H), 7.95 (s, 1H), 7.71 (s, 1H), 7.66 (d, 1H), 7.07 (s, 1H), 7.04-7.01 (m, 1H), 7.01-6.98 (m, 1H), 5.94 (quin, 1H), 4.92 (br s, 1H), 4.11 (t, 2H), 3.90 (s, 2H), 3.91-3.89 (m, 1H), 3.89 (s, 3H), 3.72 (br d, 2H), 2.49 (s, 3H), 1.70 (d, 3H). LC-MS (method 7): m/z: [M+H]⁺=440, Rₜ=0.67 min.

### Example 219

### N-{1-[5-(6,7-dihydro-5H-pyrrolo[1,2-a]imidazol-3-yl)thiophen-2-yl]ethyl}-6,7-dimethoxy-2-methylquinazolin-4-amine

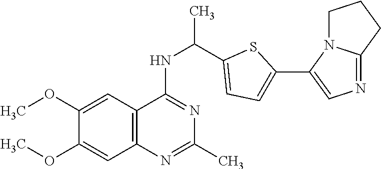 [SMILES: COc1cc2nc(C)nc(NC(C)c3ccc(-c4cnc5n4CCC5)s3)c2cc1OC]

Under argon, N-[1-(5-bromothiophen-2-yl)ethyl]-6,7-dimethoxy-2-methylquinazolin-4-amine hydrochloride (1:1) (described in example 209; 300 mg, 674 μmol), 6,7-dihydro-5H-pyrrolo[1,2-a]imidazol-3-ylboronic acid (102 mg, 674 μmol), K₂CO₃ (373 mg, 2.70 mmol) and Pd(PPh₃)₄ (39.0 mg, 33.7 μmol) in dioxane (6.9 mL) and H₂O (1.4 mL) were stirred at 110° C. overnight. 6,7-Dihydro-5H-pyrrolo[1,2-a]imidazol-3-ylboronic acid (50 mg, 330 μmol) and Pd(PPh₃)₄ (30.0 mg, 25.9 μmol) were then added again and the reaction mixture further stirred at 110° C. during 10 hours. H₂O was added, the mixture extracted with DCM, dried (hydrophobic filtration) and the solvent removed in vacuo. The crude residue was purified by preparative HPLC (basic conditions). The resulting residue was stirred in Et₂O during 20 minutes then filtered to give the title compound as a white solid (117 mg, 38%). ¹H-NMR (400 MHz, DMSO-d₆): δ [ppm]=8.11 (d, 1H), 7.63 (s, 1H), 7.03 (t, 4H), 5.89 (quin, 1H), 4.06 (t, 2H), 3.87 (s, 6H), 2.77-2.71 (m, 2H), 2.59-2.53 (m, 2H), 2.43 (s, 3H), 1.68 (d, 3H). LC-MS (method 7): m/z: [M+H]⁺=436, Rₜ=0.49 min.

### Example 220

### 6,7-dimethoxy-2-methyl-N-[1-(5-{1-[2-(pyrrolidin-1-yl)ethyl]-1H-pyrazol-4-yl}thiophen-2-yl)ethyl]quinazolin-4-amine

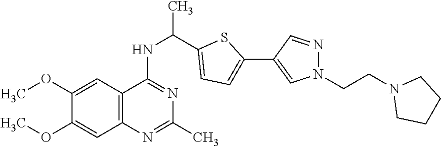 [SMILES: COc1cc2nc(C)nc(NC(C)c3ccc(-c4cnn(CCN5CCCC5)c4)s3)c2cc1OC]

Under argon, N-[1-(5-bromothiophen-2-yl)ethyl]-6,7-dimethoxy-2-methylquinazolin-4-amine (described in example 209; 50.0 mg, 122 μmol), 1-[2-(pyrrolidin-1-yl)ethyl]-4-(4,4,5,5-tetramethyl-1,3,2-dioxaborolan-2-yl)-1H-pyrazole (35.7 mg, 122 μmol), K₂CO₃ (67.7 mg, 490 μmol) and Pd(PPh₃)₄ (7.08 mg, 6.12 μmol) in dioxane (1.3 mL) and H₂O (250 μL) were stirred at 110° C. overnight. Brine was added, the mixture extracted with DCM and the solvent removed in vacuo. Purification by preparative HPLC (basic conditions) gave the title compound as a white solid (27.5 mg, 46%). ¹H-NMR (400 MHz, DMSO-d₆): δ [ppm]=8.10 (d, 1H), 7.99 (s, 1H), 7.65 (d, 2H), 7.05 (s, 1H), 7.00 (d, 1H), 6.97 (dd, 1H), 5.90 (quin, 1H), 4.17 (t, 2H), 3.88 (s, 3H), 3.87 (s, 3H), 2.81 (t, 2H), 2.47-2.40 (m, 7H), 1.68 (d, 3H), 1.64 (dt, 4H). LC-MS (method 7): m/z: [M+H]⁺=493, Rₜ=0.54 min.

### Example 221

### N-{1-[5-(1-cyclopentyl-1H-pyrazol-4-yl)thiophen-2-yl]ethyl}-6,7-dimethoxy-2-methylquinazolin-4-amine

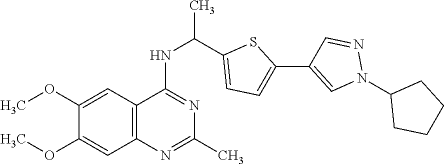 [SMILES: COc1cc2nc(C)nc(NC(C)c3ccc(-c4cnn(C5CCCC5)c4)s3)c2cc1OC]

Under argon, N-[1-(5-bromothiophen-2-yl)ethyl]-6,7-dimethoxy-2-methylquinazolin-4-amine (described in example 209; 20.0 mg, 49.0 μmol), 1-cyclopentyl-4-(4,4,5,5-tetramethyl-1,3,2-dioxaborolan-2-yl)-1H-pyrazole (12.8 mg, 49.0 μmol), K₂CO₃ (27.1 mg, 196 μmol) and Pd(PPh₃)₄ (2.83 mg, 2.45 μmol) in dioxane (500 μL) and H₂O (100 μL) were stirred at 110° C. overnight. Brine was added, the mixture extracted with DCM and the solvent removed in vacuo. Purification by preparative HPLC (acidic conditions) gave the title compound as a light yellow solid (4.70 mg, 21%). ¹H-NM R (400 MHz, DMSO-d₆): δ [ppm]=8.11 (d, 1H), 8.01 (s, 1H), 7.64 (s, 1H), 7.64 (d, 1H), 7.05 (s, 1H), 7.01 (d, 1H), 6.96 (dd, 1H), 5.90 (quin, 1H), 4.65 (quin, 1H), 3.87 (s, 3H), 3.87 (s, 3H), 2.43 (s, 3H), 2.10-2.00 (m, 2H), 1.95-1.85 (m, 2H), 1.83-1.71 (m, 2H), 1.68 (d, 3H), 1.65-1.56 (m, 2H). LC-MS (method 7): m/z: [M+H]⁺=464, Rₜ=0.95 min.

### Example 222

### 6,7-dimethoxy-2-methyl-N-{1-[5-(1H-pyrazol-3-yl)thiophen-2-yl]ethyl}quinazolin-4-amine

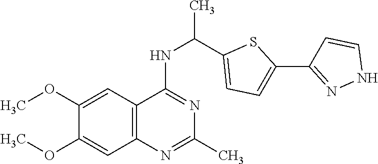 [SMILES: COc1cc2nc(C)nc(NC(C)c3ccc(-c4cc[nH]n4)s3)c2cc1OC]

Under argon, N-[1-(5-bromothiophen-2-yl)ethyl]-6,7-dimethoxy-2-methylquinazolin-4-amine (described in example 209; 20.0 mg, 49.0 μmol), 3-(4,4,5,5-tetramethyl-1,3,2-dioxaborolan-2-yl)-1H-pyrazole (9.50 mg, 49.0 μmol), K₂SO₃ (27.1 mg, 196 μmol) and Pd(PPh₃)₄ (2.83 mg, 2.45 μmol) in dioxane (500 μL) and H₂O (100 μL) were stirred at 110° C. over the weekend. 3-(4,4,5,5-tetramethyl-1,3,2-dioxaborolan-2-yl)-1H-pyrazole (9.50 mg, 49.0 μmol) and Pd(PPh₃)₄ (2.83 mg, 2.45 μmol) were then added again and the reaction mixture further stirred at 110° C. during 24 hours. Brine was added, the mixture extracted with DCM and the solvent removed in vacuo. Purification by preparative HPLC (acidic conditions) gave the title compound as an off-white solid (3.10 mg, 16%). ¹H-NMR (400 MHz, DMSO-d₆): δ [ppm]=12.78 (br s, 1H), 8.14 (s, 1H), 7.71 (br s, 1H), 7.66 (s, 1H), 7.21 (d, 1H), 7.06 (s, 1H), 7.01 (br d, 1H), 6.53 (br s, 1H), 5.92 (quin, 1H), 3.88 (s, 6H), 2.44 (s, 3H), 1.70 (d, 3H). LC-MS (method 7): m/z: [M+H]⁺=396, Rₜ=0.71 min.

### Example 223

### N-[1-(5-{2-[(3,3-difluoropyrrolidin-1-yl)methyl]phenyl}thiophen-2-yl)ethyl]-6,7-dimethoxy-2-methylquinazolin-4-amine

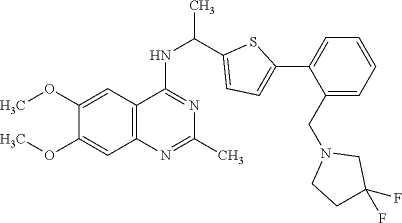 [SMILES: COc1cc2nc(C)nc(NC(C)c3ccc(-c4ccccc4CN4CCC(F)(F)C4)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), 3,3-difluoropyrrolidine hydrochloride (1:1) (33.1 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a white solid (41.6 mg, 69%). ¹H-NMR (400 MHz, DMSO-d₆): δ [ppm]=8.15 (d, 1H), 7.65 (s, 1H), 7.46-7.42 (m, 1H), 7.40-7.36 (m, 1H), 7.35 7.29 (m, 2H), 7.15 (d, 1H), 7.09 (dd, 1H), 7.05 (s, 1H), 5.97 (quin, 1H), 3.87 (s, 6H), 3.60 (s, 2H), 2.83 (t, 2H), 2.64 (t, 2H), 2.42 (s, 3H), 2.21-2.09 (m, 2H), 1.72 (d, 3H). LC-MS (method 7): m/z: [M+H]⁺=525, Rt=1.00 min.

### Example 224

### 6,7-dimethoxy-2-methyl-N-[1-(5-phenylfuran-2-yl)ethyl]quinazolin-4-amine, enantiomer

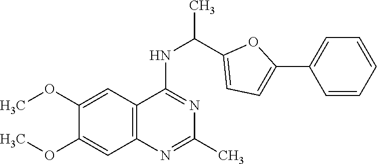 [SMILES: COc1cc2nc(C)nc(NC(C)c3ccc(-c4ccccc4)o3)c2cc1OC]

### Step a

### 1-(5-phenylfuran-2-yl)ethanamine, enantiomer 1

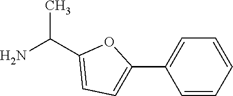

The title compound was prepared by first synthesising the sulfinamide analogous to N-[1-(5-bromothiophen-2-yl)ethyl]-2-methylpropane-2-sulfinamide (INT-28b) from 5-phenyl-2-furaldehyde (commercially available, 300 mg, 1.74 mmol), separating the two diastereoisomers obtained by preparative HPLC (Method X3) and then deprotecting diastereoisomer 1 in analogy to 1-(5-bromothiophen-2-yl)ethanamine (INT-28c) to give 43 mg of the title compound.

### Step b

### 6,7-dimethoxy-2-methyl-N-[1-(5-phenylfuran-2-yl)ethyl]quinazolin-4-amine, enantiomer

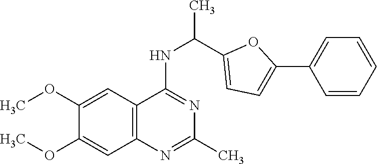 [SMILES: COc1cc2nc(C)nc(NC(C)c3ccc(-c4ccccc4)o3)c2cc1OC]

To a microwave vial were added 4-chloro-6,7-dimethoxy-2-methylquinazoline (commercially available; 35.9 mg, 151 μmol), 1-(5-phenylfuran-2-yl)ethanamine (enantiomer 1, 31.0 mg, 166 μmol), N,N-diisopropylethylamine (67 μL, 390 μmol) and DMSO (1.1 mL). The reaction mixture was heated to 130° C. during 2 hours in the microwave. Purification by preparative HPLC (acidic conditions) gave the title compound as a light yellow solid (19.0 mg, 30%). ¹H-NMR (400 MHz, DMSO-d₆): δ [ppm]=8.14 (s, 1H), 7.68 (s, 1H), 7.66 (d, 1H), 7.64 (d, 1H), 7.39 (t, 2H), 7.29 7.23 (m, 1H), 7.06 (s, 1H), 6.89 (d, 1H), 6.45 (dd, 1H), 5.85 (quin, 1H), 3.88 (s, 3H), 3.87 (s, 3H), 2.45 (s, 3H), 1.65 (d, 3H). LC-MS (method 7): m/z: [M+H]⁺=390, Rₜ=0.93 min.

### Example 225

### N-[1-(5-bromo-2,3-dihydro-1-benzofuran-7-yl)ethyl]-6,7-dimethoxy-2-methylquinazolin-4-amine

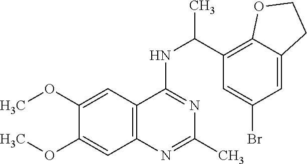 [SMILES: COc1cc2nc(C)nc(NC(C)c3cc(Br)cc4c3OCC4)c2cc1OC]

### Step a

### 1-(5-bromo-2,3-dihydro-1-benzofuran-7-yl)ethanamine, enantiomer 1

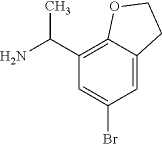

The title compound was prepared by first synthesising the sulfinamide analogous to N-[1-(5-Bromothiophen-2-yl)ethyl]-2-methylpropane-2-sulfinamide (INT-28b) from 5-bromo-2,3-dihydro-1-benzofuran-7-carbaldehyde (commercially available, 1.00 g, 4.40 mmol), separating the two diastereoisomers obtained by preparative HPLC (Method X3) and then deprotecting diastereoisomer 1 in analogy to 1-(5-bromothiophen-2-yl)ethanamine (INT-28c) to give 60 mg of the title compound.

### Step b

### N-[1-(5-bromo-2,3-dihydro-1-benzofuran-7-yl)ethyl]-6,7-dimethoxy-2-methylquinazolin-4-amine

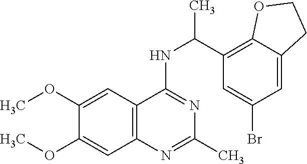 [SMILES: COc1cc2nc(C)nc(NC(C)c3cc(Br)cc4c3OCC4)c2cc1OC]

To a microwave vial were added 4-chloro-6,7-dimethoxy-2-methylquinazoline (commercially available; 48.4 mg, 203 μmol), 1-(5-bromo-2,3-dihydro-1-benzofuran-7-yl)ethanamine (enantiomer 1, 54.0 mg, 223 μmol), N,N-diisopropylethylamine (90 μL, 530 μmol) and DMSO (1.5 mL). The reaction mixture was heated to 130° C. during 2 hours in the microwave. Purification by preparative HPLC (acidic conditions) gave the title compound as a light yellow solid (33.7 mg, 36%). ¹H-NMR (400 MHz, DMSO-d₆): δ [ppm]=8.15 (s, 1H), 7.74 (s, 1H), 7.28 (d, 2H), 7.04 (s, 1H), 5.68 (quin, 1H), 4.62 (td, 2H), 3.93 (s, 3H), 3.88 (s, 3H), 3.21 (t, 2H), 2.35 (s, 3H), 1.52 (d, 3H). LC-MS (method 7): m/z: [M+H]⁺=444, Rₜ=0.90 min.

### Example 226

### 2-[1-(5-{1-[(6,7-dimethoxy-2-methylquinazolin-4-yl)amino]ethyl}furan-2-yl)-1H-pyrazol-3-yl]ethanol

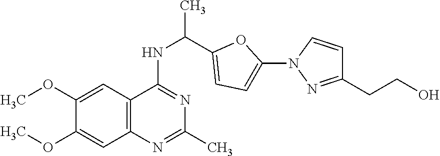 [SMILES: COc1cc2nc(C)nc(NC(C)c3ccc(-n4ccc(CCO)n4)o3)c2cc1OC]

### Step a

### 1-(5-bromo-2-furyl)ethanamine

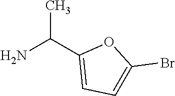

The title compound was prepared in analogy to 1-(5-bromothiophen-2-yl)ethanamine (INT-28) from 5-bromo-2-furaldehyde (commercially available, 3.00 g, 17.1 mmol) to give 300 mg of the title compound.

### Step b

### N-[1-(5-bromo-2-furyl)ethyl]-6,7-dimethoxy-2-methylquinazolin-4-amine

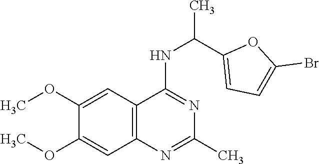 [SMILES: COc1cc2nc(C)nc(NC(C)c3ccc(Br)o3)c2cc1OC]

To a microwave vial were added 4-chloro-6,7-dimethoxy-2-methylquinazoline (commercially available; 343 mg, 1.44 mmol), 1-(5-bromo-2-furyl)ethanamine (300 mg, 1.58 mmol), N,N-diisopropylethylamine (639 μL, 3.74 mmol) and DMSO (10.0 mL). The reaction mixture was heated to 130° C. during 2 hours in the microwave. Purification by preparative HPLC (basic conditions) gave the title compound as a beige solid (227 mg, 40%). ¹H-NMR (400 MHz, DMSO-d₆): δ [ppm]=8.01 (d, 1H), 7.63 (s, 1H), 7.05 (s, 1H), 6.51 (d, 1H), 6.41 (dd, 1H), 5.75 (quin, 1H), 3.87 (s, 6H), 2.42 (s, 3H), 1.55 (d, 3H). LC-MS (method 7): m/z: [M+H]⁺=392, Rₜ=0.82 min.

### Step c

### 2-[1-(5-{1-[(6,7-dimethoxy-2-methylquinazolin-4-yl)amino]ethyl}furan-2-yl)-1H-pyrazol-3-yl]ethanol

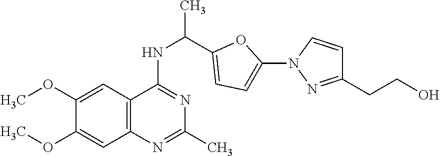 [SMILES: COc1cc2nc(C)nc(NC(C)c3ccc(-n4ccc(CCO)n4)o3)c2cc1OC]

Under argon, N-[1-(5-bromo-2-furyl)ethyl]-6,7-dimethoxy-2-methylquinazolin-4-amine (20.0 mg, 51.0 μmol), 2-(1H-pyrazol-3-yl)ethanol (5.72 mg, 51.0 μmol), Cs₂CO₃ (33.2 mg, 102 μmol) and Cu(OAc)₂ (460 μg, 2.5 μmol) in DMF (500 μL) were stirred at 110° C. for a week. The crude reaction mixture was filtered through celite. Purification by preparative HPLC (basic conditions) gave the title compound as a light yellow solid (7.60 mg, 35%). ¹H-NMR (400 MHz, DMSO-d₆): δ [ppm]=8.03 (d, 1H), 7.92 (s, 1H), 7.64 (s, 1H), 7.60 (s, 1H), 7.04 (s, 1H), 6.46 (dd, 1H), 6.34 (d, 1H), 5.79 (quin, 1H), 4.69 (t, 1H), 3.87 (s, 3H), 3.86 (s, 3H), 3.59-3.52 (m, 2H), 2.59 (t, 2H), 2.43 (s, 3H), 1.60 (d, 3H). LC-MS (method 7): m/z: [M+H]⁺=424, Rₜ=0.65 min.

### Example 227

### 5-{1-[(6,7-dimethoxy-2-methylquinazolin-4-yl)amino]ethyl}pyridin-2(1H)-one, Enantiomer 1

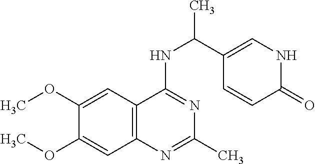 [SMILES: COc1cc2nc(C)nc(NC(C)c3ccc(=O)[nH]c3)c2cc1OC]

### Step a

### 5-[1-aminoethyl]pyridin-2(1H)-one, enantiomer 1

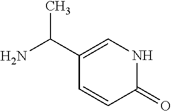

The title compound was prepared by first synthesising the sulfinamide analogous to N-[1-(5-Bromothiophen-2-yl)ethyl]-2-methylpropane-2-sulfinamide (INT-28b) from 6-oxo-1,6-dihydropyridine-3-carbaldehyde (commercially available, 1.00 g, 8.12 mmol), separating the two diastereoisomers obtained by preparative HPLC (Method X4) and then deprotecting diastereoisomer 1 in analogy to 1-(5-bromothiophen-2-yl)ethanamine (INT-28c) to give the title compound which was used directly in step b.

### Step b

### 5-{1-[(6,7-dimethoxy-2-methylquinazolin-4-yl)amino]ethyl}pyridin-2(1H)-one, Enantiomer 1

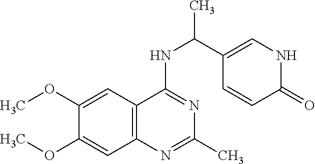 [SMILES: COc1cc2nc(C)nc(NC(C)c3ccc(=O)[nH]c3)c2cc1OC]

To a microwave vial were added 4-chloro-6,7-dimethoxy-2-methylquinazoline (commercially available; 72.5 mg, 304 μmol), 5-[1-aminoethyl]pyridin-2(1H)-one (enantiomer 1, 46.1 mg, 334 μmol), N,N-diisopropylethylamine (210 μL, 1.2 mmol) and DMSO (3.0 mL). The reaction mixture was heated to 130° C. during 2 hours in the microwave. Purification by preparative HPLC (acidic conditions followed by basic conditions) gave the title compound as a light yellow solid (11.5 mg, 11%). ¹H-NMR (400 MHz, DMSO-d₆): δ [ppm]=11.42 (br s, 1H), 7.82 (d, 1H), 7.61 (s, 1H), 7.55 (dd, 1H), 7.31 (br s, 1H), 7.03 (s, 1H), 6.32 (d, 1H), 5.39 (quin, 1H), 3.89 (s, 3H), 3.86 (s, 3H), 2.39 (s, 3H), 1.51 (d, 3H). LC-MS (method 7): m/z: [M+H]⁺=341, Rₜ=0.49 min.

### Example 228

### 6,7-dimethoxy-2-methyl-N-[1-(3-phenoxyphenyl)ethyl]quinazolin-4-amine, Enantiomer 1

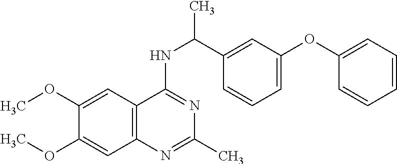 [SMILES: COc1cc2nc(C)nc(NC(C)c3cccc(Oc4ccccc4)c3)c2cc1OC]

### Step a

### 1-(3-phenoxyphenyl)ethanamine, enantiomer 1

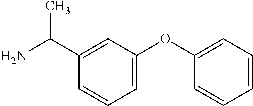 [SMILES: CC(N)c1cccc(Oc2ccccc2)c1]

The title compound was prepared by first synthesising the sulfinamide analogous to N-[1-(5-Bromothiophen-2-yl)ethyl]-2-methylpropane-2-sulfinamide (INT-28b) from 3-phenoxybenzaldehyde (commercially available, 1.00 g, 5.05 mmol), separating the two diastereoisomers obtained by preparative HPLC (Method X5) and then deprotecting diastereoisomer 1 in analogy to 1-(5-bromothiophen-2-yl)ethanamine (INT-28c) to give the title compound which was used directly in step b.

### Step b

### 6,7-dimethoxy-2-methyl-N-[1-(3-phenoxyphenyl)ethyl]quinazolin-4-amine, Enantiomer 1

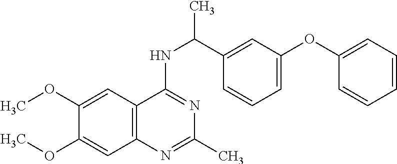 [SMILES: COc1cc2nc(C)nc(NC(C)c3cccc(Oc4ccccc4)c3)c2cc1OC]

To a microwave vial were added 4-chloro-6,7-dimethoxy-2-methylquinazoline (commercially available; 75.3 mg, 315 μmol), 1-(3-phenoxyphenyl)ethanamine (enantiomer 1, 74.0 mg, 347 μmol), N,N-diisopropylethylamine (220 μL, 1.3 mmol) and DMSO (3.2 mL). The reaction mixture was heated to 130° C. during 2 hours in the microwave. Purification by preparative HPLC (acidic conditions followed by basic conditions) gave the title compound as a light yellow solid (57.9 mg, 43%). ¹H-NM R (400 MHz, DMSO-d₆): δ [ppm]=9.12 (br s, 1H), 7.92 (s, 1H), 7.40-7.33 (m, 3H), 7.26 (d, 1H), 7.17-7.10 (m, 3H), 7.00 (br d, 1H), 6.98 (br d, 1H), 6.88-6.83 (m, 1H), 5.70 (quin, 1H), 3.93 (s, 3H), 3.92 (s, 3H), 2.46 (s, 3H), 1.64 (d, 3H). LC-MS (method 7): m/z: [M+H]⁺=416, Rₜ=0.96 min.

### Example 229

### N-[1-(2,1,3-benzothiadiazol-5-yl)ethyl]-6,7-dimethoxy-2-methylquinazolin-4-amine

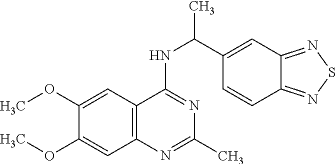 [SMILES: COc1cc2nc(C)nc(NC(C)c3ccc4nsnc4c3)c2cc1OC]

### Step a

### 1-(2,1,3-benzothiadiazol-5-yl)ethanamine

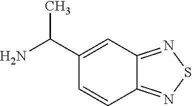

The title compound was prepared in analogy to 1-(5-bromothiophen-2-yl)ethanamine (INT-28) from 2,1,3-benzothiadiazole-5-carbaldehyde (commercially available, 500 mg, 3.05 mmol) and used directly in step b.

### Step b

### N-[1-(2,1,3-benzothiadiazol-5-yl)ethyl]-6,7-dimethoxy-2-methylquinazolin-4-amine

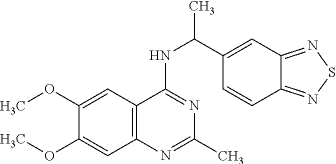 [SMILES: COc1cc2nc(C)nc(NC(C)c3ccc4nsnc4c3)c2cc1OC]

To a microwave vial were added 4-chloro-6,7-dimethoxy-2-methylquinazoline (commercially available; 23.9 mg, 100 μmol), 1-(2,1,3-benzothiadiazol-5-yl)ethanamine (19.7 mg, 110 μmol), N,N-diisopropylethylamine (68 μL, 400 μmol) and DMSO (1.0 mL). The reaction mixture was heated to 130° C. during 2 hours in the microwave. Purification by preparative HPLC (acidic conditions followed by basic conditions) gave the title compound as a light yellow solid (11.6 mg, 29%). ¹H-NMR (400 MHz, DMSO-d₆): δ [ppm]=8.30 (br s, 1H), 8.07 (d, 1H), 8.06 (s, 1H), 7.86 (dd, 1H), 7.77 (s, 1H), 7.04 (s, 1H), 5.82 (quin, 1H), 3.94 (s, 3H), 3.88 (s, 3H), 2.36 (s, 3H), 1.71 (d, 3H). LC-MS (method 7): m/z: [M+H]⁺=382, Rₜ=0.75 min.

### Example 230

### 6,7-dimethoxy-2-methyl-N-[1-(quinolin-8-yl)ethyl]quinazolin-4-amine, Enantiomer 1

### Step a

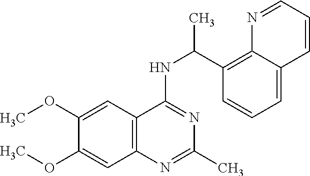 [SMILES: COc1cc2nc(C)nc(NC(C)c3cccc4cccnc34)c2cc1OC]

### 1-(quinolin-8-yl)ethanamine, Enantiomer 1

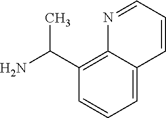

The title compound was prepared by first synthesising the sulfinamide analogous to N-[1-(5-Bromothiophen-2-yl)ethyl]-2-methylpropane-2-sulfinamide (INT-28b) from quinoline-8-carbaldehyde (commercially available, 1.00 g, 4.69 mmol), separating the two diastereoisomers obtained by preparative HPLC (Method X6) and then deprotecting diastereoisomer 1 in analogy to 1-(5-bromothiophen-2-yl)ethanamine (INT-28c) to give the title compound which was used directly in step b.

### Step b

### 6,7-dimethoxy-2-methyl-N-[1-(quinolin-8-yl)ethyl]quinazolin-4-amine, enantiomer 1

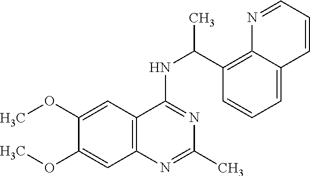 [SMILES: COc1cc2nc(C)nc(NC(C)c3cccc4cccnc34)c2cc1OC]

To a microwave vial were added 4-chloro-6,7-dimethoxy-2-methylquinazoline (commercially available; 45.6 mg, 191 μmol), 1-(quinolin-8-yl)ethanamine (enantiomer 1, 36.2 mg, 210 μmol), N,N-diisopropylethylamine (130 μL, 760 μmol) and DMSO (2.0 mL). The reaction mixture was heated to 130° C. during 2 hours in the microwave. Purification by preparative HPLC (acidic conditions followed by basic conditions) gave the title compound as a light yellow solid (6.30 mg, 9%). ¹H-NMR (400 MHz, DMSO-d₆): δ [ppm]=9.03 (dd, 1H), 8.39 (dd, 1H), 8.24 (d, 1H), 7.86 (d, 1H), 7.84-7.80 (m, 2H), 7.60 (dd, 1H), 7.56 (t, 1H), 7.02 (s, 1H), 6.74 (quin, 1H), 3.97 (s, 3H), 3.87 (s, 3H), 2.19 (s, 3H), 1.70 (d, 3H). LC-MS (method 7): m/z: [M+H]⁺=375, Rₜ=0.81 min.

### Example 231

### N-{1-[5-(cyclopent-1-en-1-yl)thiophen-2-yl]ethyl}-6,7-dimethoxy-2-methylquinazolin-4-amine

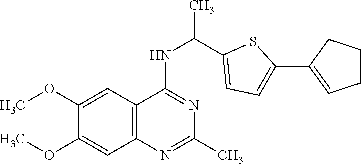 [SMILES: COc1cc2nc(C)nc(NC(C)c3ccc(C4=CCCC4)s3)c2cc1OC]

Under argon, N-[1-(5-bromothiophen-2-yl)ethyl]-6,7-dimethoxy-2-methylquinazolin-4-amine (described in example 209; 20.0 mg, 49.0 μmol), cyclopent-1-en-1-ylboronic acid (5.48 mg, 49.0 μmol), K₂SO₃ (27.1 mg, 196 μmol) and Pd(PPh₃)₄ (2.83 mg, 2.45 μmol) in dioxane (500 μL) and H₂O (100 μL) were stirred at 110° C. over the weekend. Brine was added, the mixture extracted with DCM and the solvent removed in vacuo. Purification by preparative HPLC (acidic conditions) gave the title compound as a light yellow solid (1.50 mg, 8%). ¹H-NMR (400 MHz, DMSO-d₆): δ [ppm]=8.07 (d, 1H), 7.64 (s, 1H), 7.05 (s, 1H), 6.94 (d, 1H), 6.84 (d, 1H), 5.91 5.86 (m, 1H), 3.87 (s, 6H), 2.63-2.56 (m, 2H), 2.45-2.40 (m, 5H), 1.97-1.86 (m, 2H), 1.66 (d, 3H). LC-MS (method 7): m/z: [M+H]⁺=396, Rₜ=1.01 min.

### Example 232

### N-{1-[5-(2-ethoxyphenyl)thiophen-2-yl]ethyl}-6,7-dimethoxy-2-methylquinazolin-4-amine

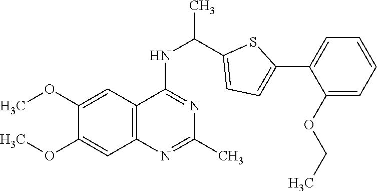 [SMILES: CCOc1ccccc1-c1ccc(C(C)Nc2nc(C)nc3cc(OC)c(OC)cc23)s1]

Under argon, N-[1-(5-bromothiophen-2-yl)ethyl]-6,7-dimethoxy-2-methylquinazolin-4-amine (described in example 209; 20.0 mg, 49.0 μmol), (2-ethoxyphenyl)boronic acid (8.13 mg, 49.0 μmol), K₂CO₃ (27.1 mg, 196 μmol) and Pd(PPh₃)₄ (2.83 mg, 2.45 μmol) in dioxane (500 μL) and H₂O (100 μL) were stirred at 110° C. overnight. Brine was added, the mixture extracted with DCM and the solvent removed in vacuo. Purification by preparative HPLC (acidic conditions) gave the title compound as an off-white solid (11.8 mg, 54%). ¹H-NMR (400 MHz, DMSO-d₆): δ [ppm]=8.12 (d, 1H), 7.66 (s, 1H), 7.64 (dd, 1H), 7.45 (d, 1H), 7.22 (td, 1H), 7.08-7.04 (m, 3H), 6.96 (td, 1H), 5.94 (quin, 1H), 4.10 (q, 2H), 3.88 (s, 3H), 3.87 (s, 3H), 2.44 (s, 3H), 1.71 (d, 3H), 1.33 (t, 3H). LC-MS (method 7): m/z: [M+H]⁺=450, Rₜ=1.00 min.

### Example 233

### N-{1-[5-(4-fluoronaphthalen-1-yl)thiophen-2-yl]ethyl}-6,7-dimethoxy-2-methylquinazolin-4-amine

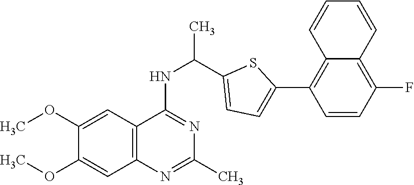 [SMILES: COc1cc2nc(C)nc(NC(C)c3ccc(-c4ccc(F)c5ccccc45)s3)c2cc1OC]

Under argon, N-[1-(5-bromothiophen-2-yl)ethyl]-6,7-dimethoxy-2-methylquinazolin-4-amine (described in example 209; 20.0 mg, 49.0 μmol), (4-fluoronaphthalen-1-yl)boronic acid (9.31 mg, 49.0 μmol), K₂SO₃ (27.1 mg, 196 μmol) and Pd(PPh₃)₄ (2.83 mg, 2.45 μmol) in dioxane (500 μL) and H₂O (100 μL) were stirred at 110° C. overnight. Brine was added, the mixture extracted with DCM and the solvent removed in vacuo. Purification by preparative HPLC (acidic conditions) gave the title compound as an off-white solid (12.6 mg, 54%). ¹H-NMR (400 MHz, DMSO-d₆): δ [ppm]=8.22-8.17 (m, 2H), 8.16-8.10 (m, 1H), 7.73-7.65 (m, 3H), 7.53 (dd, 1H), 7.37 (dd, 1H), 7.20 (dd, 1H), 7.18 (d, 1H), 7.06 (s, 1H), 6.01 (quin, 1H), 3.88 (s, 3H), 3.87 (s, 3H), 2.44 (s, 3H), 1.76 (d, 3H). LC-MS (method 7): m/z: [M+H]⁺=474, Rₜ=1.09 min.

### Example 234

### N-[1-{5-[2-(aminomethyl)-4-fluorophenyl]thiophen-2-yl}ethyl]-6,7-dimethoxy-2-methylquinazolin-4-amine

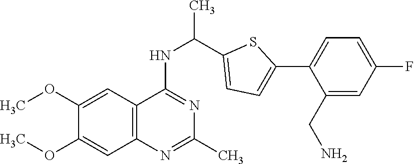 [SMILES: COc1cc2nc(C)nc(NC(C)c3ccc(-c4ccc(F)cc4CN)s3)c2cc1OC]

Under argon, N-[1-(5-bromothiophen-2-yl)ethyl]-6,7-dimethoxy-2-methylquinazolin-4-amine (described in example 209; 2.00 mg, 4.90 μmol), [2-(aminomethyl)-4-fluorophenyl]boronic acid hydrochloride (1:1) (1.01 g, 4.90 mmol), K₂CO₃ (2.71 mg, 19.6 mmol) and Pd(PPh₃)₄ (566 mg, 490 μmol) in dioxane (50 mL) and H₂O (10 mL) were stirred at 110° C. overnight. H₂O was added, the mixture extracted with DCM and the solvent removed in vacuo. Purification by column chromatography (silica gel, MeOH/EtOAc 0-20% then MeOH) gave the title compound as a white solid (1.26 g, 50%). ¹H-NMR (400 MHz, DMSO-d₆): δ [ppm]=8.25 (s, 1H), 8.16 (d, 1H), 7.65 (s, 1H), 7.45 (dd, 1H), 7.34 (dd, 1H), 7.14-7.07 (m, 2H), 7.05 (s, 1H), 7.04 (d, 1H), 5.96 (quin, 1H), 3.87 (s, 6H), 3.84 (s, 2H), 2.44 (s, 3H), 1.72 (d, 3H). LC-MS (method 7): m/z: [M+H]⁺=453, Rₜ=0.55 min.

### Example 235

### N-{1-[5-(3,6-dihydro-2H-pyran-4-yl)thiophen-2-yl]ethyl}-6,7-dimethoxy-2-methylquinazolin-4-amine

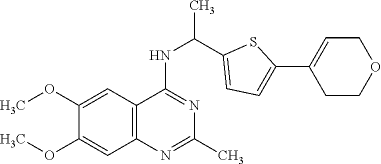 [SMILES: COc1cc2nc(C)nc(NC(C)c3ccc(C4=CCOCC4)s3)c2cc1OC]

Under argon, N-[1-(5-bromothiophen-2-yl)ethyl]-6,7-dimethoxy-2-methylquinazolin-4-amine (described in example 209; 20.0 mg, 49.0 μmol), 4-(4,4,5,5-tetramethyl-1,3,2-dioxaborolan-2-yl)-3,6-dihydro-2H-pyran (10.3 mg, 49.0 μmol), K₂CO₃ (27.1 mg, 196 μmol) and Pd(PPh₃)₄ (2.83 mg, 2.45 μmol) in dioxane (500 μL) and H₂O (100 μL) were stirred at 110° C. overnight. Brine was added, the mixture extracted with DCM and the solvent removed in vacuo. Purification by preparative HPLC (acidic conditions) gave the title compound as a light yellow solid (5.40 mg, 27%). ¹H-NM R (400 MHz, DMSO-d₆): δ [ppm]=8.08 (d, 1H), 7.64 (s, 1H), 7.05 (s, 1H), 6.96 (dd, 1H), 6.92 (d, 1H), 6.02 (br s, 1H), 5.87 (quin, 1H), 4.14 (br d, 2H), 3.87 (s, 6H), 3.76 (t, 2H), 2.42 (s, 3H), 2.41-2.36 (m, 2H), 1.66 (d, 3H). LC-MS (method 7): m/z: [M+H]⁺=412, Rₜ=0.81 min.

### Example 236

### tert-butyl {[5-(5-{1-[(6,7-dimethoxy-2-methylquinazolin-4-yl)amino]ethyl}thiophen-2-yl)furan-2-yl]methyl}carbamate

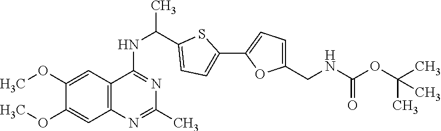 [SMILES: COc1cc2nc(C)nc(NC(C)c3ccc(-c4ccc(CNC(=O)OC(C)(C)C)o4)s3)c2cc1OC]

Under argon, N-[1-(5-bromothiophen-2-yl)ethyl]-6,7-dimethoxy-2-methylquinazolin-4-amine (described in example 209; 40.0 mg, 98.0 μmol), (5-{[(tert-butoxycarbonyl)amino]methyl}furan-2-yl)boronic acid (23.6 mg, 98.0 μmol), K₂CO₃ (54.2 mg, 392 μmol) and Pd(PPh₃)₄ (5.66 mg, 4.90 μmol) in dioxane (1.0 mL) and H₂O (200 μL) were stirred at 110° C. overnight. Brine was added, the mixture extracted with DCM and the solvent removed in vacuo. Purification by preparative HPLC (basic conditions) gave the title compound as a light yellow solid (29.0 mg, 55%). ¹H-NM R (400 MHz, DMSO-d₆): δ [ppm]=8.14 (d, 1H), 7.64 (s, 1H), 7.35 (br t, 1H), 7.14 (d, 1H), 7.06 (s, 1H), 7.03 (dd, 1H), 6.56 (d, 1H), 6.23 (d, 1H), 5.92 (quin, 1H), 4.10 (br d, 2H), 3.88 (s, 3H), 3.88-3.87 (m, 3H), 2.43 (s, 3H), 1.70 (d, 3H), 1.36 (s, 9H). LC-MS (method 7): m/z: [M+H]⁺=525, Rₜ=0.97 min.

### Example 237

### methyl 3-(5-{1-[(6,7-dimethoxy-2-methylquinazolin-4-yl)amino]ethyl}thiophen-2-yl)-1-methyl-1H-pyrazole-5-carboxylate

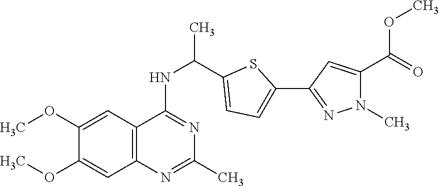

Under argon, N-[1-(5-bromothiophen-2-yl)ethyl]-6,7-dimethoxy-2-methylquinazolin-4-amine (described in example 209; 20.0 mg, 49.0 μmol), methyl 1-methyl-3-(4,4,5,5-tetramethyl-1,3,2-dioxaborolan-2-yl)-1H-pyrazole-5-carboxylate (13.0 mg, 49.0 μmol), K₂CO₃ (27.1 mg, 196 μmol) and Pd(PPh₃)₄ (2.83 mg, 2.45 μmol) in dioxane (500 μL) and H₂O (100 μL) were stirred at 110° C. overnight. Brine was added, the mixture extracted with DCM and the solvent removed in vacuo. Purification by preparative HPLC (basic conditions) gave the title compound as a white solid (1.60 mg, 7%). ¹H-NMR (400 MHz, DMSO-d₆): δ [ppm]=8.13 (d, 1H), 7.65 (s, 1H), 7.35 (d, 1H), 7.21 (s, 1H), 7.06 (s, 1H), 7.03 (dd, 1H), 5.92 (quin, 1H), 4.05 (s, 3H), 3.88 (s, 6H), 3.84 (s, 3H), 2.43 (s, 3H), 1.70 (d, 3H). LC-MS (method 7): m/z: [M+H]⁺=468, Rₜ=0.88 min.

### Example 238

### N-{1-[5-(2-{[3-(dimethylamino)pyrrolidin-1-yl]methyl}phenyl)thiophen-2-yl]ethyl}-6,7-dimethoxy-2-methylquinazolin-4-amine

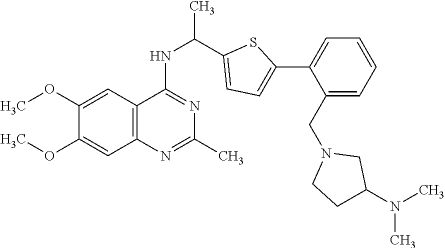

To 2-(5-{1-[(6,7-dimethoxy-2-methylquinazolin-4-yl)amino]ethyl}thiophen-2-yl)benzaldehyde (described in example 179a; 50.0 mg, 115 μmol), N,N-dimethylpyrrolidin-3-amine (26.3 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a pale yellow solid (43.5 mg, 70%). ¹H-NMR (400 MHz, DMSO-d₆): δ [ppm]=8.15 (d, 1H), 7.65 (s, 1H), 7.43-7.39 (m, 1H), 7.38-7.34 (m, 1H), 7.32-7.25 (m, 2H), 7.17 (d, 1H), 7.07 (d, 1H), 7.05 (s, 1H), 5.97 (quin, 1H), 3.87 (s, 6H), 3.54 (d, 1H), 3.46 (dd, 1H), 2.65-2.58 (m, 1H), 2.43 (s, 3H), 2.41-2.35 (m, 1H), 2.26-2.20 (m, 1H), 1.98 (s, 6H), 1.71 (d, 3H), 1.70-1.64 (m, 1H), 1.53-1.42 (m, 1H). LC-MS (method 7): m/z: [M+H]⁺=532, Rt=0.61 min.

### Example 239

### N-[1-(5-bromothiophen-3-yl)ethyl]-6,7-dimethoxy-2-methylquinazolin-4-amine

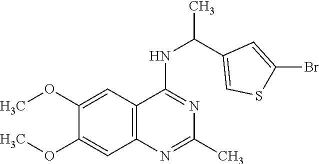 [SMILES: COc1cc2nc(C)nc(NC(C)c3csc(Br)c3)c2cc1OC]

To a microwave vial were added 4-chloro-6,7-dimethoxy-2-methylquinazoline (commercially available; 895 mg, 3.75 mmol), 1-(5-bromothiophen-3-yl)ethanamine (described in procedure INT-30; 850 mg, 4.12 mmol), N,N-diisopropylethylamine (1.7 mL, 9.7 mmol) and DMSO (19 mL). The reaction mixture was heated to 130° C. during 2.5 hours in the microwave. The mixture was diluted with H₂O, extracted with DCM and the solvent removed in vacuo. Purification by preparative HPLC (basic conditions) gave the title compound as a light brown solid (541 mg, 35%). ¹H-NMR (400 MHz, DMSO-d₆): δ [ppm]=7.94 (d, 1H), 7.64 (s, 1H), 7.41-7.35 (m, 1H), 7.24 (d, 1H), 7.04 (s, 1H), 5.70 (quin, 1H), 3.88 (s, 3H), 3.87 (s, 3H), 2.40 (s, 3H), 1.56 (d, 3H). LC-MS (method 9): m/z: [M+H]⁺=408, Rₜ=0.90 min.

### Example 240

### 2-(5-{1-[(6,7-dimethoxy-2-methylquinazolin-4-yl)amino]ethyl}thiophen-2-yl)benzamide

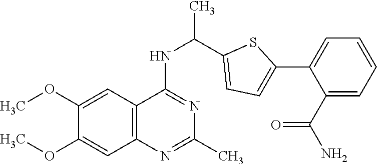 [SMILES: COc1cc2nc(C)nc(NC(C)c3ccc(-c4ccccc4C(N)=O)s3)c2cc1OC]

Under argon, N-[1-(5-bromothiophen-2-yl)ethyl]-6,7-dimethoxy-2-methylquinazolin-4-amine (described in example 209; 20 mg, 49.0 μmol), (2-carbamoylphenyl)boronic acid (8.08 mg, 49.0 μmol), K₂SO₃ (27.1 mg, 196 μmol) and Pd(PPh₃)₄ (2.83 mg, 2.45 μmol) in dioxane (500 μL) and H₂O (100 μL) were stirred at 110° C. overnight. H₂O was added, the mixture extracted with DCM and the solvent removed in vacuo. Purification by preparative HPLC (basic conditions) gave the title compound as a white solid (8.50 mg, 39%). ¹H-NMR (400 MHz, DMSO-d₆): δ [ppm]=8.16 (d, 1H), 7.79 (s, 1H), 7.65 (s, 1H), 7.44-7.38 (m, 3H), 7.35-7.32 (m, 2H), 7.13 (d, 1H), 7.06-7.04 (m, 2H), 5.95 (quin, 1H), 3.87 (s, 6H), 2.44 (s, 3H), 1.70 (d, 3H). LC-MS (method 9): m/z: [M+H]⁺=449, Rₜ=0.77 min.

### Example 241

### 3-(5-{1-[(6,7-dimethoxy-2-methylquinazolin-4-yl)amino]ethyl}thiophen-2-yl)benzamide

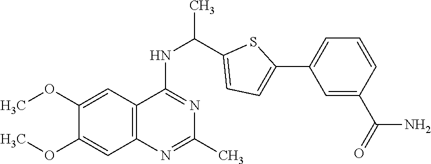 [SMILES: COc1cc2nc(C)nc(NC(C)c3ccc(-c4cccc(C(N)=O)c4)s3)c2cc1OC]

Under argon, N-[1-(5-bromothiophen-2-yl)ethyl]-6,7-dimethoxy-2-methylquinazolin-4-amine (described in example 209; 20.0 mg, 49.0 μmol), (3-carbamoylphenyl)boronic acid (8.08 mg, 49.0 μmol), K₂CO₃ (27.1 mg, 196 μmol) and Pd(PPh₃)₄ (2.83 mg, 2.45 μmol) in dioxane (500 μL) and H₂O (100 μL) were stirred at 110° C. overnight. H₂O was added, the mixture extracted with DCM and the solvent removed in vacuo. Purification by preparative HPLC (basic conditions) gave the title compound as a white solid (14.2 mg, 61%). ¹H-NMR (400 MHz, DMSO-d₆): δ [ppm]=8.16 (d, 1H), 8.08 (s, 1H), 8.06 (t, 1H), 7.78-7.71 (m, 2H), 7.66 (s, 1H), 7.48-7.42 (m, 3H), 7.11 (dd, 1H), 7.06 (s, 1H), 5.96 (quin, 1H), 3.88 (s, 6H), 2.44 (s, 3H), 1.72 (d, 3H). LC-MS (method 9): m/z: [M+H]⁺=449, Rₜ=0.81 min.

### Example 242

### 4-(5-{1-[(6,7-dimethoxy-2-methylquinazolin-4-yl)amino]ethyl}thiophen-2-yl)benzamide

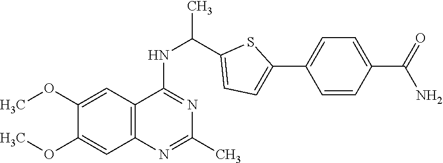 [SMILES: COc1cc2nc(C)nc(NC(C)c3ccc(-c4ccc(C(N)=O)cc4)s3)c2cc1OC]

Under argon, N-[1-(5-bromothiophen-2-yl)ethyl]-6,7-dimethoxy-2-methylquinazolin-4-amine (described in example 209; 20.0 mg, 49.0 μmol), (4-carbamoylphenyl)boronic acid (8.08 mg, 49.0 μmol), K₂CO₃ (27.1 mg, 196 μmol) and Pd(PPh₃)₄ (2.83 mg, 2.45 μmol) in dioxane (500 μL) and H₂O (100 μL) were stirred at 110° C. overnight. H₂O was added, the mixture extracted with DCM and the solvent removed in vacuo. Purification by preparative HPLC (basic conditions) gave the title compound as a white solid (3.80 mg, 17%). ¹H-NMR (400 MHz, DMSO-ds): 5 [ppm]=8.16 (d, 1H), 7.98 (s, 1H), 7.89-7.87 (m, 1H), 7.86-7.84 (m, 1H), 7.68-7.64 (m, 3H), 7.50 (d, 1H), 7.36 (s, 1H), 7.12 (dd, 1H), 7.06 (s, 1H), 5.95 (quin, 1H), 3.88 (s, 6H), 2.44 (s, 3H), 1.72 (d, 3H). LC-MS (method 9): m/z: [M+H]⁺=449, Rₜ=0.80 min.

### Example 243

### N-{1-[5-(2-aminophenyl)thiophen-2-yl]ethyl}-6,7-dimethoxy-2-methylquinazolin-4-amine

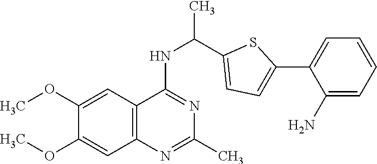 [SMILES: COc1cc2nc(C)nc(NC(C)c3ccc(-c4ccccc4N)s3)c2cc1OC]

Under argon, N-[1-(5-bromothiophen-2-yl)ethyl]-6,7-dimethoxy-2-methylquinazolin-4-amine (described in example 209; 20.0 mg, 49.0 μmol), (2-aminophenyl)boronic acid (6.71 mg, 49.0 μmol), K₂CO₃ (27.1 mg, 196 μmol) and Pd(PPh₃)₄ (2.83 mg, 2.45 μmol) in dioxane (500 μL) and H₂O (100 μL) were stirred at 110° C. overnight. H₂O was added, the mixture extracted with DCM and the solvent removed in vacuo. The crude residue was purified by preparative HPLC (basic conditions). The resulting residue was stirred in Et₂O during 2 hours then filtered to give the title compound as a light brown solid (7.50 mg, 36%). ¹H-NMR (400 MHz, DMSO-ds): 5 [ppm]=8.13 (d, 1H), 7.65 (s, 1H), 7.11-7.07 (m, 3H), 7.05 (s, 1H), 7.04-6.99 (m, 1H), 6.76 (dd, 1H), 6.57 (td, 1H), 5.95 (quin, 1H), 5.03 (s, 2H), 3.88 (s, 3H), 3.87 (s, 3H), 2.44 (s, 3H), 1.71 (d, 3H). LC-MS (method 9): m/z: [M+H]⁺=421, Rₜ=0.92 min.

### Example 244

### [2-(5-{1-[(6,7-dimethoxy-2-methylquinazolin-4-yl)amino]ethyl}thiophen-2-yl)phenyl]methanol

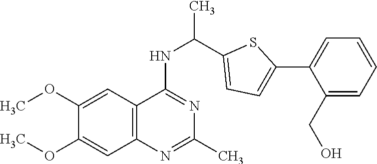 [SMILES: COc1cc2nc(C)nc(NC(C)c3ccc(-c4ccccc4CO)s3)c2cc1OC]

Under argon, N-[1-(5-bromothiophen-2-yl)ethyl]-6,7-dimethoxy-2-methylquinazolin-4-amine (described in example 209; 20.0 mg, 49.0 μmol), [2-(hydroxymethyl)phenyl]boronic acid (7.44 mg, 49.0 μmol), K₂CO₃ (27.1 mg, 196 μmol) and Pd(PPh₃)₄ (2.83 mg, 2.45 μmol) in dioxane (500 μL) and H₂O (100 μL) were stirred at 110° C. overnight. H₂O was added, the mixture extracted with DCM and the solvent removed in vacuo. Purification by preparative HPLC (basic conditions) gave the title compound as a white solid (13.2 mg, 62%). ¹H-NMR (400 MHz, DMSO-d₆): δ [ppm]=8.16 (d, 1H), 7.65 (s, 1H), 7.56 (d, 1H), 7.35 (dd, 1H), 7.33 (s, 1H), 7.30 7.25 (m, 1H), 7.12 (d, 1H), 7.09 (dd, 1H), 7.05 (s, 1H), 5.97 (quin, 1H), 5.24 (t, 1H), 4.54 (d, 2H), 3.88 (s, 6H), 2.44 (s, 3H), 1.72 (d, 3H). LC-MS (method 9): m/z: [M+H]⁺=436, Rₜ=0.88 min.

### Example 245

### 2-(5-{1-[(6,7-dimethoxy-2-methylquinazolin-4-yl)amino]ethyl}thiophen-2-yl)benzonitrile

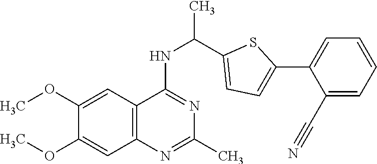 [SMILES: COc1cc2nc(C)nc(NC(C)c3ccc(-c4ccccc4C#N)s3)c2cc1OC]

Under argon, N-[1-(5-bromothiophen-2-yl)ethyl]-6,7-dimethoxy-2-methylquinazolin-4-amine (described in example 209; 20.0 mg, 49.0 μmol), (2-cyanophenyl)boronic acid (7.20 mg, 49.0 μmol), K₂SO₃ (27.1 mg, 196 μmol) and Pd(PPh₃)₄ (2.83 mg, 2.45 μmol) in dioxane (500 μL) and H₂O (100 μL) were stirred at 110° C. overnight. H₂O was added, the mixture extracted with DCM and the solvent removed in vacuo. Purification by preparative HPLC (basic conditions) gave the title compound as an off-white solid (5.00 mg, 22%). ¹H-NMR (400 MHz, DMSO-d₆): δ [ppm]=8.20 (d, 1H), 7.91 (d, 1H), 7.74-7.69 (m, 1H), 7.68-7.64 (m, 2H), 7.53-7.48 (m, 2H), 7.21 (dd, 1H), 7.06 (s, 1H), 5.97 (quin, 1H), 3.88 (s, 6H), 2.45 (s, 3H), 1.74 (d, 3H). LC-MS (method 9): m/z: [M+H]⁺=431, Rₜ=0.97 min.

### Example 246

### N-{1-[5-(1H-indazol-7-yl)thiophen-2-yl]ethyl}-6,7-dimethoxy-2-methylquinazolin-4-amine

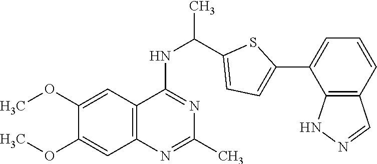 [SMILES: COc1cc2nc(C)nc(NC(C)c3ccc(-c4cccc5cn[nH]c45)s3)c2cc1OC]

Under argon, N-[1-(5-bromothiophen-2-yl)ethyl]-6,7-dimethoxy-2-methylquinazolin-4-amine (described in example 209; 20.0 mg, 49.0 μmol), 7-(4,4,5,5-tetramethyl-1,3,2-dioxaborolan-2-yl)-1H-indazole (12.0 mg, 49.0 μmol), K₂SO₃ (27.1 mg, 196 μmol) and Pd(PPh₃)₄ (2.83 mg, 2.45 μmol) in dioxane (500 μL) and H₂O (100 μL) were stirred at 110° C. overnight. H₂O was added, the mixture extracted with DCM and the solvent removed in vacuo. Purification by preparative HPLC (basic conditions) gave the title compound as a white solid (12.0 mg, 53%). ¹H-NMR (400 MHz, DMSO-d₆): δ [ppm]=13.22 (br s, 1H), 8.19 (br d, 2H), 7.72 (d, 1H), 7.67 (s, 1H), 7.55 (br d, 1H), 7.42 (br d, 1H), 7.19 (br d, 1H), 7.14 (br t, 1H), 7.06 (s, 1H), 5.99 (quin, 1H), 3.88 (s, 3H), 3.88 (s, 3H), 2.45 (s, 3H), 1.75 (d, 3H). LC-MS (method 9): m/z: [M+H]⁺=446, Rₜ=0.92 min.

### Example 247

### N-{1-[5-(1H-indazol-4-yl)thiophen-2-yl]ethyl}-6,7-dimethoxy-2-methylquinazolin-4-amine

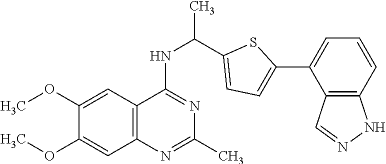 [SMILES: COc1cc2nc(C)nc(NC(C)c3ccc(-c4cccc5[nH]ncc45)s3)c2cc1OC]

Under argon, N-[1-(5-bromothiophen-2-yl)ethyl]-6,7-dimethoxy-2-methylquinazolin-4-amine (described in example 209; 20.0 mg, 49.0 μmol), 4-(4,4,5,5-tetramethyl-1,3,2-dioxaborolan-2-yl)-1H-indazole (12.0 mg, 49.0 μmol), K₂CO₃ (27.1 mg, 196 μmol) and Pd(PPh₃)₄ (2.83 mg, 2.45 μmol) in dioxane (500 μL) and H₂O (100 μL) were stirred at 110° C. overnight. H₂O was added, the mixture extracted with DCM and the solvent removed in vacuo. Purification by preparative HPLC (basic conditions) gave the title compound as a white solid (7.20 mg, 32%). ¹H-NMR (400 MHz, DMSO-d₆): δ [ppm]=13.29 (br s, 1H), 8.34 (s, 1H), 8.18 (d, 1H), 7.67 (s, 1H), 7.55 (d, 1H), 7.48 (d, 1H), 7.35 (t, 1H), 7.28 (d, 1H), 7.17 (d, 1H), 7.06 (s, 1H), 5.98 (quin, 1H), 3.88 (s, 3H), 3.88 (s, 3H), 2.46 (s, 3H), 1.75 (d, 3H). LC-MS (method 9): m/z: [M+H]⁺=446, Rₜ=0.90 min.

### Example 248

### N-{1-[5-(2-ethenylphenyl)thiophen-2-yl]ethyl}-6,7-dimethoxy-2-methylquinazolin-4-amine

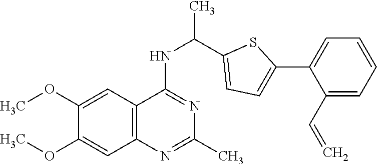 [SMILES: C=Cc1ccccc1-c1ccc(C(C)Nc2nc(C)nc3cc(OC)c(OC)cc23)s1]

Under argon, N-[1-(5-bromothiophen-2-yl)ethyl]-6,7-dimethoxy-2-methylquinazolin-4-amine (described in example 209; 200 mg, 85% purity, 416 μmol), (2-ethenylphenyl)boronic acid (61.6 mg, 416 μmol), K₂CO₃ (230 mg, 1.67 mmol) and Pd(PPh₃)₄ (48.1 mg, 41.6 μmol) in dioxane (4.0 mL) and H₂O (800 μL) were stirred at 110° C. overnight. H₂O was added, the mixture extracted with DCM and the solvent removed in vacuo. Purification by preparative HPLC (basic conditions) gave the title compound as a light yellow solid (92.9 mg, 51%). ¹H-NMR (400 MHz, DMSO-d₆): δ [ppm]=8.15 (d, 1H), 7.66-7.62 (m, 2H), 7.38-7.28 (m, 3H), 7.10 (dd, 1H), 7.05 (s, 1H), 6.91 (dd, 1H), 6.95 (d, 1H), 5.96 (quin, 1H), 5.77 (dd, 1H), 5.29 (dd, 1H), 3.87 (s, 6H), 2.43 (s, 3H), 1.72 (d, 3H). LC-MS (method 7): m/z: [M+H]⁺=432, Rt=1.09 min.

### Example 249

### 2-[4-(5-{1-[(6,7-dimethoxy-2-methylquinazolin-4-yl)amino]ethyl}thiophen-2-yl)-1H-pyrazol-1-yl]acetamide

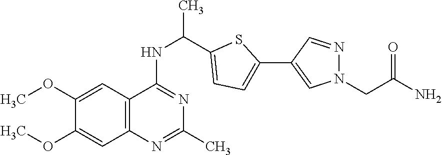 [SMILES: COc1cc2nc(C)nc(NC(C)c3ccc(-c4cnn(CC(N)=O)c4)s3)c2cc1OC]

Under argon, N-[1-(5-bromothiophen-2-yl)ethyl]-6,7-dimethoxy-2-methylquinazolin-4-amine (described in example 209; 20.0 mg, 49.0 μmol), 2-[4-(4,4,5,5-tetramethyl-1,3,2-dioxaborolan-2-yl)-1H-pyrazol-1-yl]acetamide (12.3 mg, 49.0 μmol), K₂SO₃ (27.1 mg, 196 μmol) and Pd(PPh₃)₄ (2.83 mg, 2.45 μmol) in dioxane (500 μL) and H₂O (100 μL) were stirred at 110° C. overnight. Brine was added, the mixture extracted with DCM and the solvent removed in vacuo. Purification by preparative HPLC (basic conditions) gave the title compound as a white solid (1.50 mg, 6%). ¹H-NMR (400 MHz, DMSO-d₆): δ [ppm]=8.14 (br s, 1H), 7.94 (s, 1H), 7.67 (d, 1H), 7.65 (s, 1H), 7.51 (s, 1H), 7.27 (s, 1H), 7.05 (s, 1H), 7.03 (d, 1H), 6.98 (dd, 1H), 5.91 (quin, 1H), 4.73 (s, 2H), 3.88 (s, 3H), 3.87 (s, 3H), 2.44 (s, 3H), 1.69 (d, 3H). LC-MS (method 9): m/z: [M+H]⁺=453, Rₜ=0.71 min.

### Example 250

### 6,7-dimethoxy-2-methyl-N-[1-(5-{2-[(methylamino)methyl]phenyl}thiophen-2-yl)ethyl]quinazolin-4-amine

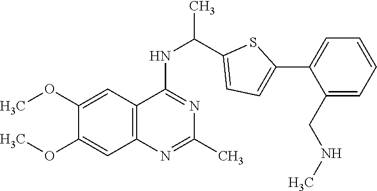 [SMILES: CNCc1ccccc1-c1ccc(C(C)Nc2nc(C)nc3cc(OC)c(OC)cc23)s1]

Under argon, N-[1-(5-bromothiophen-2-yl)ethyl]-6,7-dimethoxy-2-methylquinazolin-4-amine (described in example 209; 200 mg, 490 μmol), N-methyl-1-[2-(4,4,5,5-tetramethyl-1,3,2-dioxaborolan-2-yl)phenyl]methanamine (121 mg, 490 μmol), K₂CO₃ (271 mg, 1.96 mmol) and Pd(PPh₃)₄ (56.6 mg, 49.0 μmol) in dioxane (5.0 mL) and H₂O (1.0 mL) were stirred at 110° C. overnight. H₂O was added, the mixture extracted with DCM and the solvent removed in vacuo. Purification by preparative HPLC (basic conditions) followed by column chromatography (silica gel, MeOH/EtOAc 0-20% then MeOH) gave the title compound as a light orange solid (104 mg, 47%). ¹H-NM R (400 MHz, DMSO-d₆): δ [ppm]=8.15 (d, 1H), 7.65 (s, 1H), 7.49 (d, 1H), 7.35 7.28 (m, 2H), 7.28-7.23 (m, 1H), 7.15 (d, 1H), 7.09 (dd, 1H), 7.05 (s, 1H), 5.96 (quin, 1H), 3.87 (s, 6H), 3.65 (s, 2H), 2.44 (s, 3H), 2.25 (s, 3H), 1.72 (d, 3H). LC-MS (method 7): m/z: [M+H]⁺=449, Rₜ=0.54 min.

### Example 251

### 2-[4-(4-{1-[(6,7-dimethoxy-2-methylquinazolin-4-yl)amino]ethyl}thiophen-2-yl)-1H-pyrazol-1-yl]ethanol

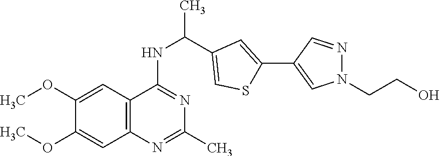 [SMILES: COc1cc2nc(C)nc(NC(C)c3csc(-c4cnn(CCO)c4)c3)c2cc1OC]

Under argon, N-[1-(5-bromothiophen-3-yl)ethyl]-6,7-dimethoxy-2-methylquinazolin-4-amine (described in example 239; 20.0 mg, 49.0 μmol), 2-[4-(4,4,5,5-tetramethyl-1,3,2-dioxaborolan-2-yl)-1H-pyrazol-1-yl]ethanol (11.7 mg, 49.0 μmol), K₂CO₃ (27.1 mg, 196 μmol) and Pd(PPh₃)₄ (2.83 mg, 2.45 μmol) in dioxane (500 μL) and H₂O (100 μL) were stirred at 110° C. overnight.

H₂O was added, the mixture extracted with DCM and the solvent removed in vacuo. Purification by preparative HPLC (basic conditions) gave the title compound as a white solid (11.8 mg, 53%). ¹H-NMR (400 MHz, DMSO-d₆): δ [ppm]=7.99 (s, 1H), 7.96 (d, 1H), 7.68 (d, 1H), 7.67 (s, 1H), 7.18 (d, 1H), 7.18-7.16 (m, 1H), 7.04 (s, 1H), 5.75 (quin, 1H), 4.91 (t, 1H), 4.12 (t, 2H), 3.88 (s, 3H), 3.87 (s, 3H), 3.73 (q, 2H), 2.41 (s, 3H), 1.59 (d, 3H). LC-MS (method 7): m/z: [M+H]⁺=440, Rₜ=0.66 min.

### Example 252

### N-[1-{5-[2-(aminomethyl)phenyl]thiophen-2-yl}ethyl]-6,7-dimethoxy-2-methylquinazolin-4-amine

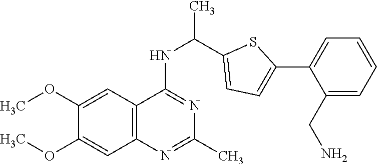 [SMILES: COc1cc2nc(C)nc(NC(C)c3ccc(-c4ccccc4CN)s3)c2cc1OC]

Under argon, N-[1-(5-bromothiophen-2-yl)ethyl]-6,7-dimethoxy-2-methylquinazolin-4-amine (described in example 209; 200 mg, 490 μmol), 1-[2-(4,4,5,5-tetramethyl-1,3,2-dioxaborolan-2-yl)phenyl]methanamine (114 mg, 490 μmol), K₂SO₃ (271 mg, 1.96 mmol) and Pd(PPh₃)₄ (56.6 mg, 49.0 μmol) in dioxane (5.0 mL) and H₂O (1.0 mL) were stirred at 110° C. overnight.

H₂O was added, the mixture extracted with DCM and the solvent removed in vacuo. Purification by column chromatography (silica gel, MeOH/EtOAc 0-20% then MeOH) gave the title compound as a pink solid (163 mg, 77%). ¹H-NMR (400 MHz, DMSO-d₆): δ [ppm]=8.15 (d, 1H), 7.65 (s, 1H), 7.56 (br d, 1H), 7.33 (td, 1H), 7.29 (dd, 1H), 7.26-7.21 (m, 1H), 7.11 (d, 1H), 7.09 (dd, 1H), 7.05 (s, 1H), 5.96 (quin, 1H), 3.87 (s, 6H), 3.77 (s, 2H), 2.44 (s, 3H), 1.72 (d, 3H). LC-MS (method 7): m/z: [M+H]⁺=435, Rₜ=0.61 min.

### Example 253

### N-{1-[5-(6,7-dihydro-5H-pyrrolo[1,2-a]imidazol-3-yl)thiophen-3-yl]ethyl}-6,7-dimethoxy-2-methylquinazolin-4-amine

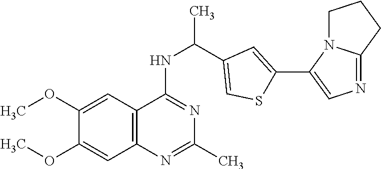 [SMILES: COc1cc2nc(C)nc(NC(C)c3csc(-c4cnc5n4CCC5)c3)c2cc1OC]

Under argon, N-[1-(5-bromothiophen-3-yl)ethyl]-6,7-dimethoxy-2-methylquinazolin-4-amine (described in example 239; 20.0 mg, 49.0 μmol), 6,7-dihydro-5H-pyrrolo[1,2-a]imidazol-3-ylboronic acid (7.44 mg, 49.0 μmol), K₂SO₃ (27.1 mg, 196 μmol) and Pd(PPh₃)₄ (2.83 mg, 2.45 μmol) in dioxane (500 μL) and H₂O (100 μL) were stirred at 110° C. overnight. H₂O was added, the mixture extracted with DCM and the solvent removed in vacuo. The crude residue was purified by preparative HPLC (basic conditions). The resulting residue was stirred in Et₂O during 2 hours then filtered to give the title compound as a pale yellow solid (4.20 mg, 19%). ¹H-NMR (400 MHz, DMSO-d₆): δ [ppm]=7.96 (d, 1H), 7.66 (s, 1H), 7.29 (t, 1H), 7.25 (d, 1H), 7.10 (s, 1H), 7.04 (s, 1H), 5.74 (quin, 1H), 4.08 (t, 2H), 3.88 (s, 3H), 3.87 (s, 3H), 2.79-2.73 (m, 2H), 2.61-2.54 (m, 2H), 2.41 (s, 3H), 1.61 (d, 3H). LC-MS (method 9): m/z: [M+H]⁺=436, Rₜ=0.56 min.

### Example 254

### 6,7-dimethoxy-2-methyl-N-[1-(5-{1-[2-(pyrrolidin-1-yl)ethyl]-1H-pyrazol-4-yl}thiophen-3-yl)ethyl]quinazolin-4-amine

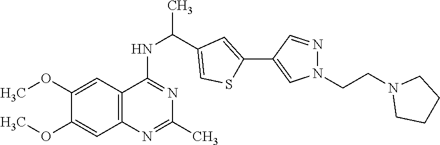 [SMILES: COc1cc2nc(C)nc(NC(C)c3csc(-c4cnn(CCN5CCCC5)c4)c3)c2cc1OC]

Under argon, N-[1-(5-bromothiophen-3-yl)ethyl]-6,7-dimethoxy-2-methylquinazolin-4-amine (described in example 239; 20.0 mg, 49.0 μmol), 1-[2-(pyrrolidin-1-yl)ethyl]-4-(4,4,5,5-tetramethyl-1,3,2-dioxaborolan-2-yl)-1H-pyrazole (14.3 mg, 49.0 μmol), K₂SO₃ (27.1 mg, 196 μmol) and Pd(PPh₃)₄ (2.83 mg, 2.45 μmol) in dioxane (500 μL) and H₂O (100 μL) were stirred at 110° C. overnight. H₂O was added, the mixture extracted with DCM and the solvent removed in vacuo. The crude residue was purified by preparative HPLC (basic conditions). The resulting residue was stirred in Et₂O during 2 hours then filtered to give the title compound as an off-white solid (800 μg, 3%). ¹H-NM R (400 MHz, DMSO-d₆): δ [ppm]=8.04 (d, 1H), 7.97 (d, 1H), 7.67 (d, 2H), 7.18-7.16 (m, 2H), 7.04 (s, 1H), 5.74 (quin, 1H), 4.18 (t, 2H), 3.88 (s, 3H), 3.87 (s, 3H), 2.81 (t, 2H), 2.46-2.42 (m, 4H), 2.41 (s, 3H), 1.64 (dt, 4H), 1.59 (d, 3H). LC-MS (method 9): m/z: [M+H]⁺=493, Rₜ=0.61 min.

### Example 255

### N-[1-{5-[2-(aminomethyl)-4-fluorophenyl]thiophen-3-yl}ethyl]-6,7-dimethoxy-2-methylquinazolin-4-amine

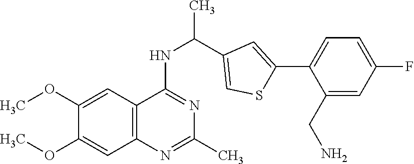 [SMILES: COc1cc2nc(C)nc(NC(C)c3csc(-c4ccc(F)cc4CN)c3)c2cc1OC]

Under argon, N-[1-(5-bromothiophen-3-yl)ethyl]-6,7-dimethoxy-2-methylquinazolin-4-amine (described in example 239; 50.0 mg, 122 μmol), [2-(aminomethyl)-4-fluorophenyl]boronic acid hydrochloride (1:1) (25.2 mg, 122 μmol), K₂CO₃ (67.7 mg, 490 μmol) and Pd(PPh₃)₄ (7.08 mg, 6.12 μmol) in dioxane (1.2 mL) and H₂O (250 μL) were stirred at 110° C. during 36 hours. H₂O was added, the mixture extracted with DCM and the solvent removed in vacuo. Purification by preparative HPLC (basic conditions) gave the title compound as a pale yellow solid (17.4 mg, 31%). ¹H-NM R (400 MHz, DMSO-d₆): δ [ppm]=7.98 (d, 1H), 7.66 (s, 1H), 7.44 (dd, 1H), 7.41 (t, 1H), 7.34 (dd, 1H), 7.20 (d, 1H), 7.08 (td, 1H), 7.03 (s, 1H), 5.77 (quin, 1H), 3.87 (s, 3H), 3.86 (s, 3H), 3.76 (s, 2H), 2.41 (s, 3H), 1.62 (d, 3H). LC-MS (Method 8): m/z: [M+H]⁺=453, Rₜ=0.69 min.

### Example 256

### 2-[4-(3-{(1R)-1-[(6,7-dimethoxy-2-methylquinazolin-4-yl)amino]ethyl}phenyl)-1H-pyrazol-1-yl]ethanol

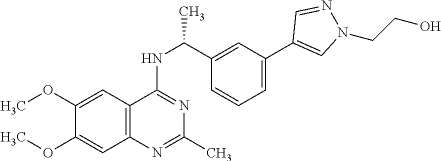 [SMILES: COc1cc2nc(C)nc(N[C@H](C)c3cccc(-c4cnn(CCO)c4)c3)c2cc1OC]

Under argon, N-[(1R)-1-(3-bromophenyl)ethyl]-6,7-dimethoxy-2-methylquinazolin-4-amine (described in example 191; 100 mg, 249 μmol), 2-[4-(4,4,5,5-tetramethyl-1,3,2-dioxaborolan-2-yl)-1H-pyrazol-1-yl]ethanol (59.2 mg, 249 μmol), K₂CO₃ (137 mg, 994 μmol) and Pd(PPh₃)₄ (14.4 mg, 12.4 μmol) in dioxane (2.5 mL) and H₂O (500 μL) were stirred at 110° C. overnight.

H₂O was added, the mixture extracted with DCM and the solvent removed in vacuo. Purification preparative HPLC (basic conditions) gave the title compound as a pale yellow solid (10.4 mg, 10%). ¹H-NM R (400 MHz, DMSO-d₆): δ [ppm]=8.11 (d, 1H), 7.99 (d, 1H), 7.84 (d, 1H), 7.71 (s, 1H), 7.65 (s, 1H), 7.41 (dt, 1H), 7.30 (d, 1H), 7.28-7.24 (m, 1H), 7.02 (s, 1H), 5.67 (quin, 1H), 4.92 (t, 1H), 4.15 (t, 2H), 3.90 (s, 3H), 3.86 (s, 3H), 3.75 (q, 2H), 2.36 (s, 3H), 1.61 (d, 3H). LC-MS (Method 8): m/z: [M+H]⁺=434, Rₜ=0.81 min.

### Example 257

### 6,7-dimethoxy-2-methyl-N-[(1R)-1-(3-{1-[2-(pyrrolidin-1-yl)ethyl]-1H-pyrazol-4-yl}phenyl)ethyl]quinazolin-4-amine

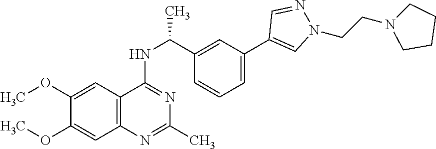 [SMILES: COc1cc2nc(C)nc(N[C@H](C)c3cccc(-c4cnn(CCN5CCCC5)c4)c3)c2cc1OC]

Under argon, N-[(1R)-1-(3-bromophenyl)ethyl]-6,7-dimethoxy-2-methylquinazolin-4-amine (described in example 191; 100 mg, 249 μmol), 1-[2-(pyrrolidin-1-yl)ethyl]-4-(4,4,5,5-tetramethyl-1,3,2-dioxaborolan-2-yl)-1H-pyrazole (72.4 mg, 249 μmol), K₂CO₃ (137 mg, 994 μmol) and Pd(PPh₃)₄ (14.4 mg, 12.4 μmol) in dioxane (2.5 mL) and H₂O (500 μL) were stirred at 110° C. overnight. H₂O was added, the mixture extracted with DCM and the solvent removed in vacuo. Purification preparative HPLC (basic conditions) gave the title compound as a white solid (73.2 mg, 59%). ¹H-NM R (400 MHz, DMSO-d₆): δ [ppm]=8.14 (s, 1H), 7.98 (d, 1H), 7.83 (d, 1H), 7.71 (s, 1H), 7.64 (br s, 1H), 7.41 (dt, 1H), 7.30 (d, 1H), 7.28-7.24 (m, 1H), 7.02 (s, 1H), 5.67 (quin, 1H), 4.21 (t, 2H), 3.90 (s, 3H), 3.86 (s, 3H), 2.82 (t, 2H), 2.47-2.41 (m, 4H), 2.36 (s, 3H), 1.66-1.59 (m, 7H). LC-MS (Method 8): m/z: [M+H]⁺=487, Rₜ=0.68 min.

### Example 258

### N-{(1R)-1-[2′-(aminomethyl)biphenyl-3-yl]ethyl}-6,7-dimethoxy-2-methylquinazolin-4-amine

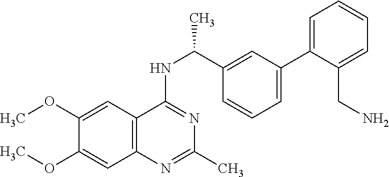 [SMILES: COc1cc2nc(C)nc(N[C@H](C)c3cccc(-c4ccccc4CN)c3)c2cc1OC]

Under argon, N-[(1R)-1-(3-bromophenyl)ethyl]-6,7-dimethoxy-2-methylquinazolin-4-amine (described in example 191; 100 mg, 249 μmol), 1-[2-(4,4,5,5-tetramethyl-1,3,2-dioxaborolan-2-yl)phenyl]methanamine (57.9 mg, 249 μmol), K₂CO₃ (137 mg, 994 μmol) and Pd(PPh₃)₄ (28.7 mg, 24.9 μmol) in dioxane (1.3 mL) and H₂O (250 μL) were stirred at 110° C. overnight.

H₂O was added, the mixture extracted with DCM and the solvent removed in vacuo. Purification by column chromatography (silica gel, MeOH/EtOAc 0-20% then MeOH) gave the title compound as a yellow solid (95.0 mg, 88%). ¹H-NMR (400 MHz, DMSO-d₆): δ [ppm]=8.03 (d, 1H), 7.69 (s, 1H), 7.58 (d, 1H), 7.48 (br d, 1H), 7.45-7.34 (m, 5H), 7.26-7.21 (m, 2H), 7.01 (s, 1H), 5.71 (quin, 1H), 3.89 (s, 3H), 3.86 (s, 3H), 3.81-3.77 (m, 2H), 2.35 (s, 3H), 1.63 (d, 3H).

LC-MS (method 7): m/z: [M+H]⁺=429, Rₜ=0.55 min.

### Example 259

### N-[1-{5-[2-(aminomethyl)phenyl]thiophen-3-yl}ethyl]-6,7-dimethoxy-2-methylquinazolin-4-amine

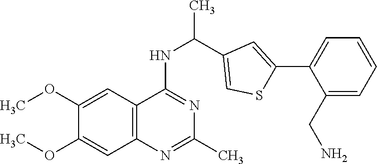 [SMILES: COc1cc2nc(C)nc(NC(C)c3csc(-c4ccccc4CN)c3)c2cc1OC]

Under argon, N-[1-(5-bromothiophen-3-yl)ethyl]-6,7-dimethoxy-2-methylquinazolin-4-amine (described in example 239; 50.0 mg, 122 μmol), 1-[2-(4,4,5,5-tetramethyl-1,3,2-dioxaborolan-2-yl)phenyl]methanamine (28.5 mg, 122 μmol), K₂CO₃ (67.7 mg, 490 μmol) and Pd(PPh₃)₄ (14.2 mg, 12.2 μmol) in dioxane (1.3 mL) and H₂O (250 μL) were stirred at 110° C. overnight. H₂O was added, the mixture extracted with DCM and the solvent removed in vacuo. Purification by column chromatography (silica gel, MeOH/EtOAc 0-20% then MeOH) gave the title compound as a yellow solid (33.1 mg, 59%). ¹H-NMR (400 MHz, DMSO-d₆): δ [ppm]=7.99 (d, 1H), 7.66 (s, 1H), 7.57 (br d, 1H), 7.41 (t, 1H), 7.37-7.30 (m, 2H), 7.28-7.25 (m, 2H), 7.03 (s, 1H), 5.78 (quin, 1H), 3.87 (s, 3H), 3.86 (s, 3H), 3.79 (s, 2H), 2.41 (s, 3H), 1.63 (d, 3H). LC-MS (method 7): m/z: [M+H]⁺=435, Rₜ=0.55 min.

### Example 260

### N-{(1R)-1-[3-(aminomethyl)phenyl]ethyl}-6,7-dimethoxy-2-methylquinazolin-4-amine

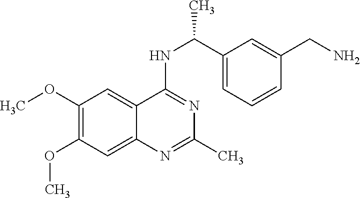 [SMILES: COc1cc2nc(C)nc(N[C@H](C)c3cccc(CN)c3)c2cc1OC]

To 3-{(1R)-1-[(6,7-dimethoxy-2-methylquinazolin-4-yl)amino]ethyl}benzonitrile (described in example 194; 300 mg, 861 μmol) and NiCl₂·6H₂O (40.9 mg, 172 μmol) in MeOH (9.0 mL) was carefully added NaBH₄ (163 mg, 4.31 mmol) and the reaction mixture stirred 10 minutes at room temperature. H₂O was added carefully, the mixture extracted with DCM and the solvent removed in vacuo. Purification by column chromatography (silica gel, MeOH/EtOAc 0-20% then MeOH) gave the title compound as a white solid (140 mg, 46%). ¹H-NMR (400 MHz, DMSO-d₆): δ [ppm]=7.97 (d, 1H), 7.70 (s, 1H), 7.41 (s, 1H), 7.28 (dt, 1H), 7.24 (t, 1H), 7.18 (br d, 1H), 7.01 (s, 1H), 5.65 (quin, 1H), 3.89 (s, 3H), 3.86 (s, 3H), 3.69 (s, 2H), 2.35 (s, 3H), 1.57 (d, 3H). LC-MS (method 7): m/z: [M+H]⁺=353, Rₜ=0.41 min.

### Example 261

### N-{(1R)-1-[3-(6,7-dihydro-5H-pyrrolo[1,2-a]imidazol-3-yl)phenyl]ethyl}-6,7-dimethoxy-2-methylquinazolin-4-amine

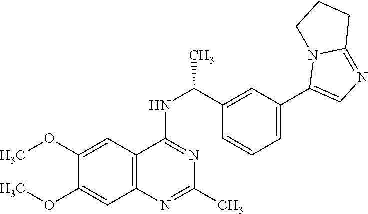 [SMILES: COc1cc2nc(C)nc(N[C@H](C)c3cccc(-c4cnc5n4CCC5)c3)c2cc1OC]

Under argon, N-[(1R)-1-(3-bromophenyl)ethyl]-6,7-dimethoxy-2-methylquinazolin-4-amine (described in example 191; 100 mg, 249 μmol), 6,7-dihydro-5H-pyrrolo[1,2-a]imidazol-3-ylboronic acid (37.8 mg, 249 μmol), K₂SO₃ (137 mg, 994 μmol) and Pd(PPh₃)₄ (28.7 mg, 24.9 μmol) in dioxane (2.5 mL) and H₂O (500 μL) were stirred at 110° C. overnight. H₂O was added, the mixture extracted with DCM and the solvent removed in vacuo. Purification preparative HPLC (basic conditions) gave the title compound as a yellow solid (26.9 mg, 25%). ¹H-NMR (400 MHz, DMSO-d₆): δ [ppm]=8.02 (d, 1H), 7.71 (s, 1H), 7.60 (s, 1H), 7.41-7.32 (m, 3H), 7.17 (s, 1H), 7.02 (s, 1H), 5.68 (quin, 1H), 4.10 (qt, 2H), 3.90 (s, 3H), 3.86 (s, 3H), 2.78-2.72 (m, 2H), 2.56-2.52 (m, 2H), 2.35 (s, 3H), 1.61 (d, 3H). LC-MS (method 7): m/z: [M+H]⁺=430, Rₜ=0.52 min.

### Example 262

### N-[1-(5-{2-[(dimethylamino)methyl]phenyl}thiophen-2-yl)ethyl]-6,7-dimethoxy-2-methylquinazolin-4-amine

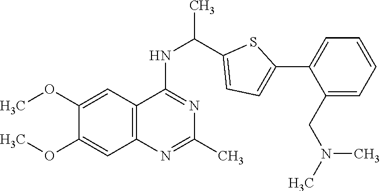 [SMILES: COc1cc2nc(C)nc(NC(C)c3ccc(-c4ccccc4CN(C)C)s3)c2cc1OC]

Under argon, N-[1-(5-bromothiophen-2-yl)ethyl]-6,7-dimethoxy-2-methylquinazolin-4-amine (described in example 209; 20.0 mg, 49.0 μmol), {2-[(dimethylamino)methyl]phenyl}boronic acid (8.77 mg, 49.0 μmol), K₂SO₃ (27.1 mg, 196 μmol) and Pd(PPh₃)₄ (5.66 mg, 4.90 μmol) in dioxane (500 μL) and H₂O (100 μL) were stirred at 110° C. overnight. H₂O was added, the mixture extracted with DCM, dried (hydrophobic filtration) and the solvent removed in vacuo. Purification by preparative HPLC (basic conditions) gave the title compound as a white solid (12.5 mg, 55%). ¹H-NMR (400 MHz, DMSO-d₆): δ [ppm]=8.16 (d, 1H), 7.65 (s, 1H), 7.43-7.40 (m, 1H), 7.38-7.34 (m, 1H), 7.31-7.27 (m, 2H), 7.17 (d, 1H), 7.08 (dd, 1H), 7.04 (s, 1H), 5.96 (quin, 1H), 3.86 (s, 6H), 2.43 (s, 3H), 2.10 (s, 6H), 1.71 (d, 3H). LC-MS (method 7): m/z: [M+H]⁺=463, Rₜ=0.60 min.

### Example 263

### 6,7-dimethoxy-2-methyl-N-[1-(5-{2-[(methylamino)methyl]phenyl}thiophen-3-yl)ethyl]quinazolin-4-amine

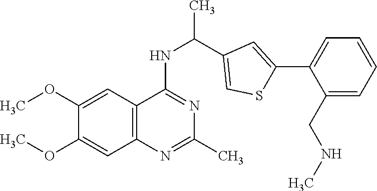 [SMILES: CNCc1ccccc1-c1cc(C(C)Nc2nc(C)nc3cc(OC)c(OC)cc23)cs1]

Under argon, N-[1-(5-bromothiophen-3-yl)ethyl]-6,7-dimethoxy-2-methylquinazolin-4-amine (described in example 239; 50.0 mg, 122 μmol), N-methyl-1-[2-(4,4,5,5-tetramethyl-1,3,2-dioxaborolan-2-yl)phenyl]methanamine (30.3 mg, 122 μmol), K₂CO₃ (67.7 mg, 490 μmol) and Pd(PPh₃)₄ (14.2 mg, 12.2 μmol) in dioxane (1.3 mL) and H₂O (250 μL) were stirred at 110° C. overnight. H₂O was added, the mixture extracted with DCM and the solvent removed in vacuo. Purification by preparative HPLC (basic conditions) gave the title compound as a pale yellow solid (30.9 mg, 56%). ¹H-NM R (400 MHz, DMSO-d₆): δ [ppm]=8.01 (d, 1H), 7.66 (s, 1H), 7.46 (dd, 1H), 7.41 (t, 1H), 7.38-7.34 (m, 2H), 7.34-7.25 (m, 2H), 7.02 (s, 1H), 5.79 (quin, 1H), 3.86 (s, 6H), 3.60 (s, 2H), 2.40 (s, 3H), 2.15 (s, 3H), 1.63 (d, 3H). LC-MS (method 7): m/z: [M+H]⁺=449, Rₜ=0.59 min.

### Example 264

### N-[1-(4-bromothiophen-2-yl)ethyl]-6,7-dimethoxy-2-methylquinazolin-4-amine

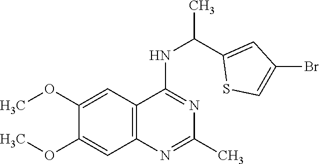 [SMILES: COc1cc2nc(C)nc(NC(C)c3cc(Br)cs3)c2cc1OC]

To a microwave vial were added 4-chloro-6,7-dimethoxy-2-methylquinazoline (commercially available; 500 mg, 2.09 mmol), 1-(4-bromothiophen-2-yl)ethanamine hydrochloride (1:1) (described in procedure INT-29; 559 mg, 2.30 mmol), N,N-diisopropylethylamine (930 μL, 5.4 mmol) and DMSO (4.0 mL). The reaction mixture was heated to 130° C. during 6 hours in the microwave. H₂O was added, the mixture extracted with DCM, dried (hydrophobic filtration) and the solvent removed in vacuo. The crude residue was stirred in Et₂O during 30 minutes then filtered to give the title compound as a pale yellow solid (668 mg, 71%). ¹H-NMR (400 MHz, DMSO-d₆): δ [ppm]=8.09 (d, 1H), 7.61 (s, 1H), 7.48 (d, 1H), 7.07 (t, 1H), 7.05 (s, 1H), 5.87 (quin, 1H), 3.87 (s, 6H), 2.41 (s, 3H), 1.67 (d, 3H). LC-MS (method 7): m/z: [M+H]⁺=408, Rₜ=0.86 min.

### Example 265

### N-[1-{5-[3-(aminomethyl)phenyl]thiophen-2-yl}ethyl]-6,7-dimethoxy-2-methylquinazolin-4-amine

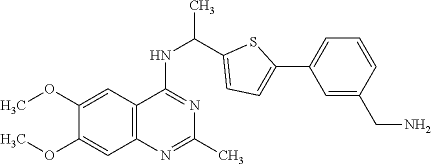 [SMILES: COc1cc2nc(C)nc(NC(C)c3ccc(-c4cccc(CN)c4)s3)c2cc1OC]

Under argon, N-[1-(5-bromothiophen-2-yl)ethyl]-6,7-dimethoxy-2-methylquinazolin-4-amine (described in example 209; 100 mg, 225 μmol), 1-[3-(4,4,5,5-tetramethyl-1,3,2-dioxaborolan-2-yl)phenyl]methanamine hydrochloride (1:1) (60.6 mg, 225 μmol), K₂CO₃ (124 mg, 899 μmol) and Pd(PPh₃)₄ (13.0 mg, 11.2 μmol) in dioxane (2.3 mL) and H₂O (460 μL) were stirred at 110° C. overnight. H₂O was added, the mixture extracted with DCM, dried (hydrophobic filtration) and the solvent removed in vacuo. Purification by column chromatography (silica gel, MeOH/EtOAc 0-20% then MeOH) gave the title compound as a white solid (78.6 mg, 79%). ¹H-NMR (400 MHz, DMSO-d₆): δ [ppm]=8.15 (d, 1H), 7.65 (s, 1H), 7.54 (s, 1H), 7.42-7.39 (m, 1H), 7.34 (d, 1H), 7.28 (t, 1H), 7.21 (br d, 1H), 7.06 (dd, 1H), 7.05 (s, 1H), 5.93 (quin, 1H), 3.87 (s, 6H), 3.70 (s, 2H), 2.43 (s, 3H), 1.70 (d, 3H). LC-MS (method 7): m/z: [M+H]⁺=435, Rₜ=0.57 min.

### Example 266

### N-[1-{5-[4-(aminomethyl)phenyl]thiophen-2-yl}ethyl]-6,7-dimethoxy-2-methylquinazolin-4-amine

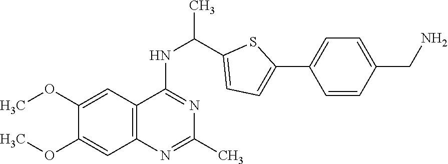 [SMILES: COc1cc2nc(C)nc(NC(C)c3ccc(-c4ccc(CN)cc4)s3)c2cc1OC]

Under argon, N-[1-(5-bromothiophen-2-yl)ethyl]-6,7-dimethoxy-2-methylquinazolin-4-amine (described in example 209; 100 mg, 225 μmol), [4-(aminomethyl)phenyl]boronic acid hydrochloride (1:1) (42.1 mg, 225 μmol), K₂CO₃ (124 mg, 899 μmol) and Pd(PPh₃)₄ (13.0 mg, 11.2 μmol) in dioxane (2.3 mL) and H₂O (460 μL) were stirred at 110° C. overnight. H₂O was added, the mixture extracted with DCM and the solvent removed in vacuo. Purification by column chromatography (silica gel, MeOH/EtOAc 0-20% then MeOH) gave the title compound as a white solid (48.5 mg, 47%). ¹H-NMR (400 MHz, DMSO-d₆): δ [ppm]=8.13 (d, 1H), 7.64 (s, 1H), 7.53-7.47 (m, 2H), 7.34-7.28 (m, 3H), 7.07-7.03 (m, 2H), 5.92 (quin, 1H), 3.87 (s, 6H), 3.68 (s, 2H), 2.43 (s, 3H), 1.69 (d, 3H). LC-MS (method 7): m/z: [M+H]⁺=435, Rₜ=0.57 min.

### Example 267

### 6,7-dimethoxy-2-methyl-N-[1-(4-{1-[2-(pyrrolidin-1-yl)ethyl]-1H-pyrazol-3-yl}thiophen-2-yl)ethyl]quinazolin-4-amine

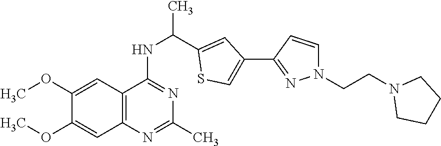 [SMILES: COc1cc2nc(C)nc(NC(C)c3cc(-c4ccn(CCN5CCCC5)n4)cs3)c2cc1OC]

Under argon, −[1-(4-bromothiophen-2-yl)ethyl]-6,7-dimethoxy-2-methylquinazolin-4-amine (described in example 264; 50.0 mg, 122 μmol), 1-[2-(pyrrolidin-1-yl)ethyl]-4-(4,4,5,5-tetramethyl-1,3,2-dioxaborolan-2-yl)-1H-pyrazole (35.7 mg, 122 μmol), K₂SO₃ (67.7 mg, 490 μmol) and Pd(PPh₃)₄ (14.2 mg, 12.2 μmol) in dioxane (1.3 mL) and H₂O (250 μL) were stirred at 110° C. overnight. H₂O was added, the mixture extracted with DCM and the solvent removed in vacuo. Purification by column chromatography (silica gel, MeOH/EtOAc 0-20% then MeOH) gave the title compound as a white solid (32.5 mg, 53%). ¹H-NMR (400 MHz, DMSO-d₆): δ [ppm]=8.10 (d, 1H), 8.04 (d, 1H), 7.73 (d, 1H), 7.64 (s, 1H), 7.33 (d, 1H), 7.28 (t, 1H), 7.04 (s, 1H), 5.92 (quin, 1H), 4.18 (t, 2H), 3.87 (s, 3H), 3.86 (s, 3H), 2.85-2.76 (m, 2H), 2.45-2.41 (m, 7H), 1.70 (d, 3H), 1.64 (dt, 4H). LC-MS (method 7): m/z: [M+H]⁺=493, Rₜ=0.55 min.

### Example 268

### 6,7-dimethoxy-2-methyl-N-[(1R)-1-{2′-[(methylamino)methyl]biphenyl-3-yl}ethyl]quinazolin-4-amine

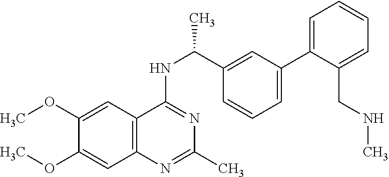

Under argon, N-[(1R)-1-(3-bromophenyl)ethyl]-6,7-dimethoxy-2-methylquinazolin-4-amine (described in example 191; 100 mg, 249 μmol), N-methyl-1-[2-(4,4,5,5-tetramethyl-1,3,2-dioxaborolan-2-yl)phenyl]methanamine (61.4 mg, 249 μmol), K₂CO₃ (137 mg, 994 μmol) and Pd(PPh₃)₄ (28.7 mg, 24.9 μmol) in dioxane (1.3 mL) and H₂O (250 μL) were stirred at 110° C. overnight. H₂O was added, the mixture extracted with DCM and the solvent removed in vacuo. Purification by preparative HPLC (basic conditions) followed by column chromatography (silica gel, MeOH/EtOAc 0-20% then MeOH) gave the title compound as a white solid (32.0 mg, 28%). ¹H-NMR (400 MHz, DMSO-d₆): δ [ppm]=8.01 (d, 1H), 7.69 (s, 1H), 7.49 (dd, 2H), 7.44 (br d, 1H), 7.38 (t, 1H), 7.30 (dtd, 2H), 7.23 (dt, 1H), 7.19 (dd, 1H), 7.01 (s, 1H), 5.69 (quin, 1H), 3.88 (s, 3H), 3.86 (s, 3H), 3.45 (s, 2H), 2.34 (s, 3H), 2.08 (s, 3H), 1.62 (d, 3H). LC-MS (method 7): m/z: [M+H]⁺=443, Rₜ=0.58 min.

### Example 269

### N-[1-{4-[2-(aminomethyl)-4-fluorophenyl]thiophen-2-yl}ethyl]-6,7-dimethoxy-2-methylquinazolin-4-amine

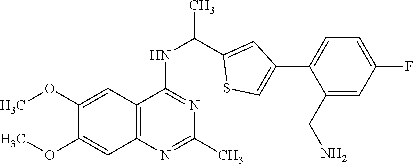 [SMILES: COc1cc2nc(C)nc(NC(C)c3cc(-c4ccc(F)cc4CN)cs3)c2cc1OC]

Under argon, −[1-(4-bromothiophen-2-yl)ethyl]-6,7-dimethoxy-2-methylquinazolin-4-amine (described in example 264; 50.0 mg, 122 μmol), [2-(aminomethyl)-4-fluorophenyl]boronic acid hydrochloride (1:1) (25.2 mg, 122 μmol), K₂SO₃ (67.7 mg, 490 μmol) and Pd(PPh₃)₄ (14.2 mg, 12.2 μmol) in dioxane (1.3 mL) and H₂O (250 μL) were stirred at 110° C. overnight. H₂O was added, the mixture extracted with DCM and the solvent removed in vacuo. Purification by column chromatography (silica gel, MeOH/EtOAc 0-20% then MeOH) gave the title compound as a white solid (32.2 mg, 55%). ¹H-NMR (400 MHz, DMSO-d₆): δ [ppm]=8.14 (d, 1H), 7.64 (s, 1H), 7.40 (dd, 1H), 7.36 (d, 1H), 7.27 (dd, 1H), 7.17 (t, 1H), 7.09-7.03 (m, 2H), 5.95 (quin, 1H), 3.87 (s, 6H), 3.71 (s, 2H), 2.43 (s, 3H), 1.71 (d, 3H). LC-MS (method 9): m/z: [M+H]⁺=453, Rₜ=0.70 min.

### Example 270

### N-[1-{4-[2-(aminomethyl)phenyl]thiophen-2-yl}ethyl]-6,7-dimethoxy-2-methylquinazolin-4-amine

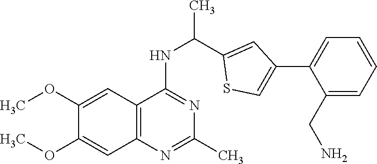 [SMILES: COc1cc2nc(C)nc(NC(C)c3cc(-c4ccccc4CN)cs3)c2cc1OC]

Under argon, −[1-(4-bromothiophen-2-yl)ethyl]-6,7-dimethoxy-2-methylquinazolin-4-amine (described in example 264; 50.0 mg, 122 μmol), 1-[2-(4,4,5,5-tetramethyl-1,3,2-dioxaborolan-2-yl)phenyl]methanamine (28.5 mg, 122 μmol), K₂CO₃ (67.7 mg, 490 μmol) and Pd(PPh₃)₄ (14.2 mg, 12.2 μmol) in dioxane (1.3 mL) and H₂O (250 μL) were stirred at 110° C. overnight. H₂O was added, the mixture extracted with DCM and the solvent removed in vacuo. Purification by column chromatography (silica gel, MeOH/EtOAc 0-20% then MeOH) gave the title compound as a white solid (5.20 mg, 9%). ¹H-NMR (400 MHz, DMSO-d₆): δ [ppm]=8.15 (d, 1H), 7.65 (s, 1H), 7.53 (d, 1H), 7.41 (d, 1H), 7.33-7.28 (m, 1H), 7.27-7.23 (m, 2H), 7.21 (t, 1H), 7.05 (s, 1H), 5.96 (quin, 1H), 3.87 (s, 6H), 3.71 (s, 2H), 2.43 (s, 3H), 1.72 (d, 3H). LC-MS (method 9): m/z: [M+H]⁺=435, Rₜ=0.69 min.

### Example 271

### 6,7-dimethoxy-2-methyl-N-[1-(4-{2-[(methylamino)methyl]phenyl}thiophen-2-yl)ethyl]quinazolin-4-amine

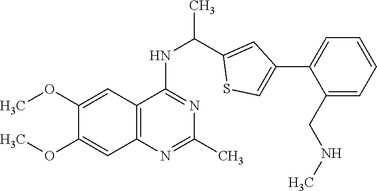 [SMILES: CNCc1ccccc1-c1csc(C(C)Nc2nc(C)nc3cc(OC)c(OC)cc23)c1]

Under argon, −[1-(4-bromothiophen-2-yl)ethyl]-6,7-dimethoxy-2-methylquinazolin-4-amine (described in example 264; 50.0 mg, 122 μmol), N-methyl-1-[2-(4,4,5,5-tetramethyl-1,3,2-dioxaborolan-2-yl)phenyl]methanamine (30.3 mg, 122 μmol), K₂SO₃ (67.7 mg, 490 μmol) and Pd(PPh₃)₄ (14.2 mg, 12.2 μmol) in dioxane (1.3 mL) and H₂O (250 μL) were stirred at 110° C. overnight. H₂O was added, the mixture extracted with DCM and the solvent removed in vacuo. Purification by column chromatography (silica gel, MeOH/EtOAc 0-20% then MeOH) gave the title compound as a white solid (15.4 mg, 28%). ¹H-NMR (400 MHz, DMSO-d₆): δ [ppm]=8.14 (d, 1H), 7.65 (s, 1H), 7.47-7.43 (m, 2H), 7.32-7.25 (m, 4H), 7.05 (s, 1H), 5.96 (quin, 1H), 3.87 (s, 3H), 3.87 (s, 3H), 3.56 (s, 2H), 2.43 (s, 3H), 2.22 (s, 3H), 1.72 (d, 3H). LC-MS (method 7): m/z: [M+H]⁺=449, Rₜ=0.71 min.

### Example 272

### N-{1-[4-(6,7-dihydro-5H-pyrrolo[1,2-a]imidazol-3-yl)thiophen-2-yl]ethyl}-6,7-dimethoxy-2-methylquinazolin-4-amine

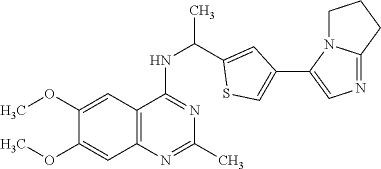 [SMILES: COc1cc2nc(C)nc(NC(C)c3cc(-c4cnc5n4CCC5)cs3)c2cc1OC]

Under argon, −[1-(4-bromothiophen-2-yl)ethyl]-6,7-dimethoxy-2-methylquinazolin-4-amine (described in example 264; 50.0 mg, 122 μmol), 6,7-dihydro-5H-pyrrolo[1,2-a]imidazol-3-ylboronic acid (18.6 mg, 122 μmol), K₂CO₃ (67.7 mg, 490 μmol) and Pd(PPh₃)₄ (14.2 mg, 12.2 μmol) in dioxane (1.3 mL) and H₂O (250 μL) were stirred at 110° C. overnight. H₂O was added, the mixture extracted with DCM and the solvent removed in vacuo. Purification by preparative HPLC (basic conditions) gave the title compound as a white solid (16.0 mg, 30%). ¹H-NMR (400 MHz, DMSO-d₆): δ [ppm]=8.11 (d, 1H), 7.63 (s, 1H), 7.35 (d, 1H), 7.31 (t, 1H), 7.13 (s, 1H), 7.04 (s, 1H), 5.91 (quin, 1H), 4.11 (t, 2H), 3.87 (s, 6H), 2.77-2.71 (m, 2H), 2.61-2.52 (m, 2H), 2.43 (s, 3H), 1.71 (d, 3H). LC-MS (method 7): m/z: [M+H]⁺=436, Rₜ=0.49 min.

### Example 273

### 2-[3-(5-{1-[(6,7-dimethoxy-2-methylquinazolin-4-yl)amino]ethyl}thiophen-3-yl)-1H-pyrazol-1-yl]ethanol

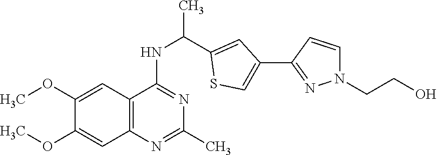 [SMILES: COc1cc2nc(C)nc(NC(C)c3cc(-c4ccn(CCO)n4)cs3)c2cc1OC]

Under argon, −[1-(4-bromothiophen-2-yl)ethyl]-6,7-dimethoxy-2-methylquinazolin-4-amine (described in example 264; 50.0 mg, 122 μmol), 2-[4-(4,4,5,5-tetramethyl-1,3,2-dioxaborolan-2-yl)-1H-pyrazol-1-yl]ethanol (29.2 mg, 122 μmol), K₂CO₃ (67.7 mg, 490 μmol) and Pd(PPh₃)₄ (14.2 mg, 12.2 μmol) in dioxane (1.3 mL) and H₂O (250 μL) were stirred at 110° C. overnight. H₂O was added, the mixture extracted with DCM and the solvent removed in vacuo. Purification by preparative HPLC (basic conditions) gave the title compound as a white solid (28.1 mg, 51%). ¹H-NMR (400 MHz, DMSO-d₆): δ [ppm]=8.11 (d, 1H), 8.00 (d, 1H), 7.74 (s, 1H), 7.64 (s, 1H), 7.33 (d, 1H), 7.28 (t, 1H), 7.04 (s, 1H), 5.91 (quin, 1H), 4.92 (t, 1H), 4.11 (t, 2H), 3.86 (d, 6H), 3.73 (q, 2H), 2.42 (s, 3H), 1.70 (d, 3H). LC-MS (method 7): m/z: [M+H]⁺=440, Rₜ=0.67 min.

### Example 274

### N-{(1R)-1-[2′-(aminomethyl)-4′-fluorobiphenyl-3-yl]ethyl}-6,7-dimethoxy-2-methylquinazolin-4-amine

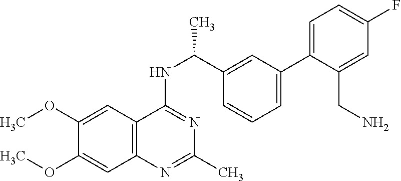 [SMILES: COc1cc2nc(C)nc(N[C@H](C)c3cccc(-c4ccc(F)cc4CN)c3)c2cc1OC]

Under argon, N-[(1R)-1-(3-bromophenyl)ethyl]-6,7-dimethoxy-2-methylquinazolin-4-amine (described in example 191; 100 mg, 249 μmol), [2-(aminomethyl)-4-fluorophenyl]boronic acid hydrochloride (1:1) (51.1 mg, 249 μmol), K₂CO₃ (137 mg, 994 μmol) and Pd(PPh₃)₄ (14.4 mg, 12.4 μmol) in dioxane (2.5 mL) and H₂O (500 μL) were stirred at 110° C. overnight. H₂O was added, the mixture extracted with DCM and the solvent removed in vacuo. Purification preparative HPLC (basic conditions) followed twice by column chromatography (silica gel, MeOH/EtOAc 0-20% then MeOH) gave the title compound as a white solid (15.6 mg, 14%). ¹H-NMR (400 MHz, DMSO-d₆): δ [ppm]=8.00 (d, 1H), 7.68 (s, 1H), 7.47-7.36 (m, 4H), 7.23-7.16 (m, 2H), 7.13-7.06 (m, 1H), 7.02 (s, 1H), 5.68 (quin, 1H), 3.89 (s, 3H), 3.86 (s, 3H), 3.65-3.54 (m, 2H), 2.35 (s, 3H), 1.62 (d, 3H). LC-MS (method 7): m/z: [M+H]⁺=447, Rₜ=0.57 min.

### Example 275

### N-[1-{5-[5-(aminomethyl)furan-2-yl]thiophen-2-yl}ethyl]-6,7-dimethoxy-2-methylquinazolin-4-amine

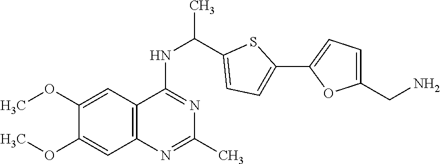 [SMILES: COc1cc2nc(C)nc(NC(C)c3ccc(-c4ccc(CN)o4)s3)c2cc1OC]

To tert-butyl {[5-(5-{1-[(6,7-dimethoxy-2-methylquinazolin-4-yl)amino]ethyl}thiophen-2-yl)furan-2-yl]methyl}carbamate (described in example 236; 110 mg, 210 μmol) in MeOH (4.2 mL) was added dropwise acetyl chloride (45 μL, 630 μmol) and the reaction mixture stirred at room temperature over the weekend. The reaction was quenched with NaHCO₃ (sat.), diluted with H₂O, the mixture extracted with DCM and the solvent removed in vacuo. Purification by column chromatography (MeOH/EtOAc 0-20% then MeOH) followed by preparative HPLC (basic conditions) gave the title compound as an off-white solid (17.5 mg, 18%). ¹H-NMR (400 MHz, DMSO-d₆): δ [ppm]=8.14 (d, 1H), 7.64 (s, 1H), 7.14 (d, 1H), 7.05 (s, 1H), 7.02 (d, 1H), 6.56 (d, 1H), 6.28 (d, 1H), 5.91 (quin, 1H), 3.87 (s, 3H), 3.87 (s, 3H), 3.71 (s, 2H), 2.42 (s, 3H), 1.69 (d, 3H). LC-MS (method 7): m/z: [M+H]⁺=425, Rₜ=0.51 min.

### Example 276

### N-{1-[5′-(aminomethyl)-2,2′-bithiophen-5-yl]ethyl}-6,7-dimethoxy-2-methylquinazolin-4-amine

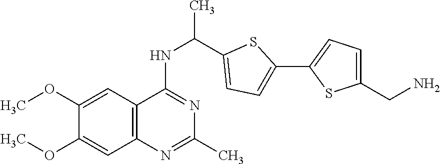 [SMILES: COc1cc2nc(C)nc(NC(C)c3ccc(-c4ccc(CN)s4)s3)c2cc1OC]

### Step a

### tert-butyl [(5′-{1-[(6,7-dimethoxy-2-methylquinazolin-4-yl)amino]ethyl}-2,2′-bithiophen-5-yl)methyl]carbamate

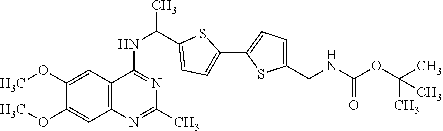 [SMILES: COc1cc2nc(C)nc(NC(C)c3ccc(-c4ccc(CNC(=O)OC(C)(C)C)s4)s3)c2cc1OC]

Under argon, N-[1-(5-bromo-2-thienyl)ethyl]-6,7-dimethoxy-2-methylquinazolin-4-amine (described in example 209; 20.0 mg, 50.0 μmol), (5-{[(tert-butoxycarbonyl)amino]methyl}-2-thienyl)boronic acid (12.6 mg, 50.0 μmol), K₂SO₃ (27.1 mg, 196 μmol) and Pd(PPh₃)₄ (2.83 mg, 2.44 μmol) in dioxane (500 μL) and H₂O (100 μL) were stirred at 110° C. overnight. H₂O was added, the mixture extracted with DCM and the solvent removed in vacuo. Purification preparative HPLC (basic conditions) gave the title compound as a light yellow solid (15.6 mg, 14%). ¹H-NM R (400 MHz, DMSO-d₆): δ [ppm]=8.12 (d, 1H), 7.64 (s, 1H), 7.50 (t, 1H), 7.07 (d, 1H), 7.06 (s, 1H), 7.03-7.00 (m, 2H), 6.82 (d, 1H), 5.89 (quin, 1H), 4.22 (br d, 2H), 3.88 (s, 6H), 2.43 (s, 3H), 1.69 (d, 3H), 1.39 (s, 9H). LC-MS (method 9): m/z: [M+H]⁺=541, Rt=1.09 min.

### Step b

### N-{1-[5′-(aminomethyl)-2,2′-bithiophen-5-yl]ethyl}-6,7-dimethoxy-2-methylquinazolin-4-amine

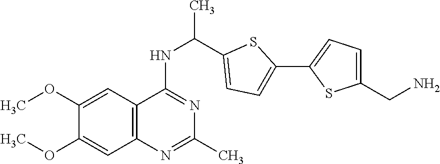 [SMILES: COc1cc2nc(C)nc(NC(C)c3ccc(-c4ccc(CN)s4)s3)c2cc1OC]

To tert-butyl [(5′-{1-[(6,7-dimethoxy-2-methylquinazolin-4-yl)amino]ethyl}-2,2′-bithiophen-5-yl)methyl]carbamate (100 mg, 185 μmol) in MeOH (3.7 mL) was added dropwise acetyl chloride (79 μL, 1.1 mmol) and the reaction mixture stirred at room temperature over the weekend. The reaction was quenched with NaHCO₃(sat.), diluted with H₂O, the mixture extracted with DCM and the solvent removed in vacuo. Purification by column chromatography (MeOH/EtOAc 0-20% then MeOH) followed by preparative HPLC (basic conditions) gave the title compound as a yellow solid (39.6 mg, 47%). 1H-NMR (400 MHz, DMSO-d₆): δ [ppm]=8.12 (d, 1H), 7.64 (s, 1H), 7.06 (d, 1H), 7.05 (s, 1H), 7.03 (d, 1H), 7.00 (dd, 1H), 6.86 (d, 1H), 5.88 (quin, 1H), 3.91-3.88 (m, 2H), 3.87 (s, 6H), 2.43 (s, 3H), 1.68 (d, 3H). LC-MS (method 7): m/z: [M+H]⁺=441, Rt=0.53 min.

### Example 277

### 2-{[2-(5-{1-[(6,7-dimethoxy-2-methylquinazolin-4-yl)amino]ethyl}thiophen-2-yl)benzyl]amino}-1-(1H-indol-3-yl)ethanone

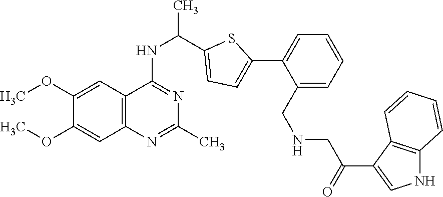 [SMILES: COc1cc2nc(C)nc(NC(C)c3ccc(-c4ccccc4CNCC(=O)c4c[nH]c5ccccc45)s3)c2cc1OC]

Under argon, 2-(5-{1-[(6,7-Dimethoxy-2-methylquinazolin-4-yl)amino]ethyl}thiophen-2-yl)benzaldehyde (described in example 179a; 50.0 mg, 115 μmol) and 2-amino-1-(1H-indol-3-yl)ethanone hydrochloride (1:1) (24.3 mg, 115 μmol) in MeOH (1.2 mL) were stirred at room temperature overnight. NaBH(OAc)₃ (48.9 mg, 231 μmol) was then added and the reaction mixture stirred at room temperature during 15 minutes. The reaction was quenched with NaOH (1.0 M), the mixture extracted with DCM and the solvent removed in vacuo. Purification by column chromatography (MeOH/EtOAc 0-50% then MeOH) followed by preparative HPLC (acidic conditions) gave the title compound as an off-white solid (2.20 mg, 3%). 1H-NMR (400 MHz, DMSO-d₆): δ [ppm]=11.97 (br s, 1H), 8.33 (d, 1H), 8.29-8.21 (m, 1H), 8.19-8.12 (m, 2H), 7.63 (s, 1H), 7.59 (d, 1H), 7.46 (dd, 1H), 7.37-7.31 (m, 2H), 7.31-7.25 (m, 1H), 7.24-7.15 (m, 3H), 7.05-7.02 (m, 2H), 5.94 (quin, 1H), 3.94-3.90 (m, 2H), 3.86 (s, 3H), 3.85 (s, 3H), 3.84 3.81 (m, 2H), 2.42 (s, 3H), 1.69 (d, 3H). LC-MS (method 7): m/z: [M+H]⁺=592, Rₜ=0.67 min.

### Example 278

### 3-amino-4-(5-{1-[(6,7-dimethoxy-2-methylquinazolin-4-yl)amino]ethyl}thiophen-2-yl)-1-benzothiophene-2-carboxamide

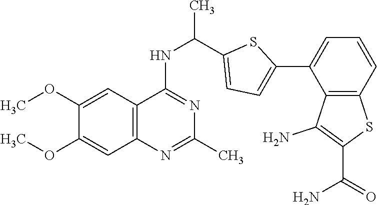 [SMILES: COc1cc2nc(C)nc(NC(C)c3ccc(-c4cccc5sc(C(N)=O)c(N)c45)s3)c2cc1OC]

### Step a

### (5-{1-[(6,7-dimethoxy-2-methylquinazolin-4-yl)amino]ethyl}-2-thienyl)boronic acid

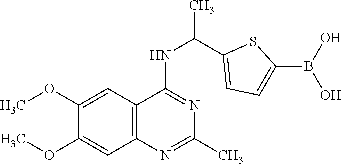 [SMILES: COc1cc2nc(C)nc(NC(C)c3ccc(B(O)O)s3)c2cc1OC]

Under argon, N-[1-(5-bromo-2-thienyl)ethyl]-6,7-dimethoxy-2-methylquinazolin-4-amine (described in example 209; 500 mg, 1.23 mmol), 4,4,4′,4′,5,5,5′,5′-octamethyl-2,2′-bi-1,3,2-dioxaborolane (466 mg, 1.84 mg), KOAc (361 mg, 3.67 mmol) and PdCl₂(dppf) (89.6 mg, 122 μmol) in dioxane (12.5 mL) were stirred at 80° C. overnight) and the solvent then removed in vacuo. Purification by preparative HPLC (basic conditions) gave the title compound as a white solid (157 mg, 34%). ¹H-NMR (400 MHz, DMSO-d₆): δ [ppm]=8.09 (s, 2H), 7.64 (s, 1H), 7.50 (d, 1H), 7.08 (dd, 1H), 7.04 (s, 1H), 6.55 (s, 1H), 5.95 (quin, 1H), 3.86 (s, 6H), 2.40 (s, 3H), 1.74-1.61 (m, 3H). LC-MS (method 7): m/z: [M+H]⁺=374, Rₜ=0.59 min.

### Step b

### 3-amino-4-(5-{1-[(6,7-dimethoxy-2-methylquinazolin-4-yl)amino]ethyl}thiophen-2-yl)-1-benzothiophene-2-carboxamide

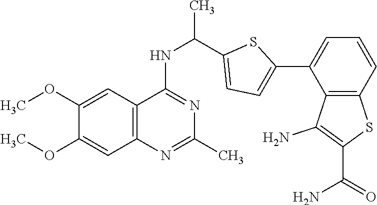 [SMILES: COc1cc2nc(C)nc(NC(C)c3ccc(-c4cccc5sc(C(N)=O)c(N)c45)s3)c2cc1OC]

Under argon, (5-{1-[(6,7-dimethoxy-2-methylquinazolin-4-yl)amino]ethyl}thiophen-2-yl)boronic acid (30.0 mg, 80.4 μmol), 3-amino-4-bromo-1-benzothiophene-2-carboxamide (21.8 mg, 80.4 μmol), K₂CO₃ (44.4 mg, 322 μmol) and Pd(PPh₃)₄ (9.29 mg, 8.04 μmol) in dioxane (500 μL) and H₂O (100 μL) were stirred at 110° C. overnight. H₂O was added, the mixture extracted with DCM and the solvent removed in vacuo. Purification by preparative HPLC (basic conditions, repeated twice) gave the title compound as a pale yellow solid (1.00 mg, 2%). 1H-NMR (400 MHz, DMSO-d₆): δ [ppm]=8.20 (d, 1H), 7.90 (dd, 1H), 7.65 (s, 1H), 7.44 (dd, 1H), 7.22 (dd, 1H), 7.19-7.13 (m, 3H), 7.05 (t, 2H), 6.19-6.10 (m, 2H), 5.97 (quin, 1H), 3.89-3.87 (m, 3H), 3.87-3.86 (m, 3H), 2.43 (s, 3H), 1.75 (d, 3H). LC-MS (method 7): m/z: [M+H]⁺=520, Rt=0.86 min.

### Example 279

### 2-[2-(5-{1-[(6,7-dimethoxy-2-methylquinazolin-4-yl)amino]ethyl}thiophen-2-yl)-benzyl]glycinamide

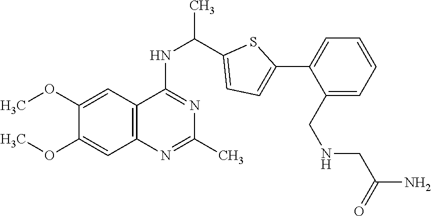 [SMILES: COc1cc2nc(C)nc(NC(C)c3ccc(-c4ccccc4CNCC(N)=O)s3)c2cc1OC]

A solution of 2-(5-{1-[(6,7-dimethoxy-2-methylquinazolin-4-yl)amino]ethyl}thiophen-2-yl)benzaldehyde (described in example 179a; 50.0 mg, 115 μmol) and glycinamide hydrochloride (1:1) (12.7 mg, 115 μmol) in MeOH (1.2 mL) was stirred at room temperature overnight. NaBH(OAc)₃ (48.9 mg, 231 μmol) was added and the solution stirred at room temperature during 15 nm. The reaction was quenched with aqueous NaOH (1.0 M, 1.0 mL), the mixture extracted with DCM and the solvent removed in vacuo. Purification by column chromatography (silica gel, MeOH/EtOAc 0-50% then MeOH) followed by preparative HPLC (basic conditions) gave the title compound as a white solid (28.5 mg, 47%). 1H-NMR (400 MHz, DMSO-d₆): δ [ppm]=8.15 (d, 1H), 7.64 (s, 1H), 7.54 (d, 1H), 7.35-7.30 (m, 2H), 7.29 7.25 (m, 1H), 7.25-7.21 (m, 1H), 7.12 (d, 1H), 7.07 (dd, 1H), 7.05 (s, 1H), 7.01 (br s, 1H), 5.96 (quin, 1H), 3.87 (s, 6H), 3.72 (s, 2H), 3.03 (s, 2H), 2.43 (s, 3H), 1.71 (d, 3H). LC-MS (method 7): m/z: [M+H]⁺=492, Rt=0.55 min.

### Example 280

### 2-[2-(5-{1-[(6,7-dimethoxy-2-methylquinazolin-4-yl)amino]ethyl}thiophen-2-yl)benzyl]-N,N-dimethylglycinamide

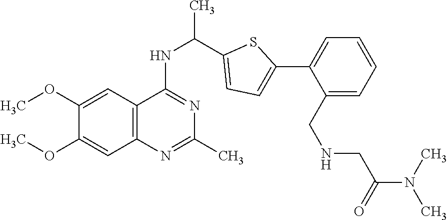

A solution of 2-(5-{1-[(6,7-dimethoxy-2-methylquinazolin-4-yl)amino]ethyl}thiophen-2-yl)benzaldehyde (described in example 179a; 50.0 mg, 115 μmol) and N,N-dimethylglycinamide (11.8 mg, 115 μmol) in MeOH (1.2 mL) was stirred at room temperature overnight. NaBH(OAc)₃ (48.9 mg, 231 μmol) was added and the solution stirred at room temperature during 15 mn. The reaction was quenched with aqueous NaOH (1.0 M, 1.0 mL), the mixture extracted with DCM and the solvent removed in vacuo. Purification by column chromatography (silica gel, MeOH/EtOAc 0-50% then MeOH) followed by preparative HPLC (basic conditions) gave the title compound as a white solid (28.5 mg, 47%). 1H-NMR (400 MHz, DMSO-d₆): δ [ppm]=8.15 (d, 1H), 7.65 (s, 1H), 7.54-7.49 (m, 1H), 7.35-7.30 (m, 2H), 7.29-7.24 (m, 1H), 7.15 (d, 1H), 7.08 (dd, 1H), 7.05 (s, 1H), 5.96 (quin, 1H), 3.87 (s, 6H), 3.73 (s, 2H), 3.30 (s, 2H), 2.83 (s, 3H), 2.79 (s, 3H), 2.43 (s, 3H), 1.71 (d, 3H). LC-MS (method 7): m/z: [M+H]⁺=520, Rt=0.57 min.

### Example 281

### methyl N-[2-(5-{1-[(6,7-dimethoxy-2-methylquinazolin-4-yl)amino]ethyl}thiophen-2-yl)benzyl]glycinate

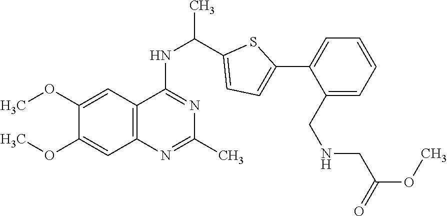

A solution of 2-(5-{1-[(6,7-dimethoxy-2-methylquinazolin-4-yl)amino]ethyl}thiophen-2-yl)benzaldehyde (described in example 179a; 50.0 mg, 115 μmol) and methyl glycinate hydrochloride (1:1) (14.5 mg, 115 μmol) in MeOH (1.2 mL) was stirred at room temperature overnight. NaBH(OAc)₃ (48.9 mg, 231 μmol) was added and the solution stirred at room temperature during 15 mn. The reaction was quenched with aqueous NaOH (1.0 M, 1.0 mL), the mixture extracted with DCM and the solvent removed in vacuo. Purification by column chromatography (silica gel, MeOH/EtOAc 0-50% then MeOH) followed by preparative HPLC (basic conditions) gave the title compound as a white solid (28.5 mg, 47%). 1H-NMR (400 MHz, DMSO-d₆): δ [ppm]=8.15 (d, 1H), 7.65 (s, 1H), 7.51-7.48 (m, 1H), 7.34-7.29 (m, 2H), 7.29-7.24 (m, 1H), 7.15 (d, 1H), 7.08 (dd, 1H), 7.04 (s, 1H), 5.96 (quin, 1H), 3.87 (s, 6H), 3.74 (s, 2H), 3.57 (s, 3H), 3.31 (s, 2H), 2.43 (s, 3H), 1.71 (d, 3H). LC-MS (method 7): m/z: [M+H]⁺=507, Rt=0.60 min.

### Example 282

### 2-[2-(5-{1-[(6,7-dimethoxy-2-methylquinazolin-4-yl)amino]ethyl}thiophen-2-yl)benzyl]-N-methylglycinamide

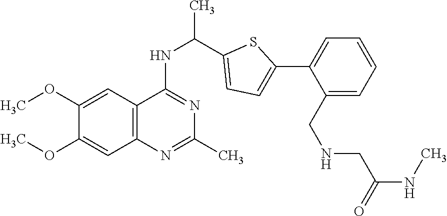

A solution of 2-(5-{1-[(6,7-dimethoxy-2-methylquinazolin-4-yl)amino]ethyl}thiophen-2-yl)benzaldehyde (described in example 179a; 50.0 mg, 115 μmol) and N-methylglycinamide hydrochloride (1:1) (14.4 mg, 115 μmol) in MeOH (1.2 mL) was stirred at room temperature during 5 hours. NaBH(OAc)₃ (48.9 mg, 231 μmol) was added and the solution stirred at room temperature during 15 mn. The reaction was quenched with aqueous NaOH (1.0 M, 1.0 mL), the mixture extracted with DCM and the solvent removed in vacuo. Purification by column chromatography (silica gel, MeOH/EtOAc 0-50% then MeOH) followed by preparative HPLC (basic conditions) gave the title compound as a white solid (26.0 mg, 45%). 1H-NMR (400 MHz, DMSO-d₆): δ [ppm]=8.15 (d, 1H), 7.67 (br d, 1H), 7.64 (s, 1H), 7.55 (d, 1H), 7.35-7.29 (m, 2H), 7.29-7.24 (m, 1H), 7.10 (d, 1H), 7.08 (dd, 1H), 7.05 (s, 1H), 5.96 (quin, 1H), 3.87 (s, 6H), 3.70 (s, 2H), 3.04 (s, 2H), 2.56 (d, 3H), 2.43 (s, 3H), 1.71 (d, 3H). LC-MS (method 7): m/z: [M+H]⁺=506, Rt=0.56 min.

### Example 283

### 2-[2-(5-{1-[(6,7-dimethoxy-2-methylquinazolin-4-yl)amino]ethyl}thiophen-2-yl)benzyl]-N-(2-methoxyethyl)glycinamide

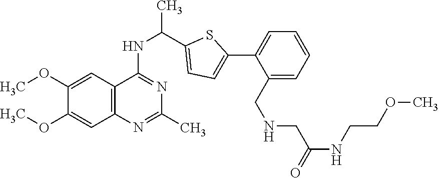

A solution of 2-(5-{1-[(6,7-dimethoxy-2-methylquinazolin-4-yl)amino]ethyl}thiophen-2-yl)benzaldehyde (described in example 179a; 50.0 mg, 115 μmol) and N-(2-methoxyethyl)glycinamide hydrochloride (1:1) (19.4 mg, 115 μmol) in MeOH (1.2 mL) was stirred at room temperature during 5 hours. NaBH(OAc)₃ (48.9 mg, 231 μmol) was added and the solution stirred at room temperature during 15 mn. The reaction was quenched with aqueous NaOH (1.0 M, 1.0 mL), the mixture extracted with DCM and the solvent removed in vacuo. Purification by column chromatography (silica gel, MeOH/EtOAc 0-50% then MeOH) followed by preparative HPLC (basic conditions) gave the title compound as a white solid (15.5 mg, 24%). 1H-NMR (400 MHz, DMSO-d₆): δ [ppm]=8.15 (d, 1H), 7.80 (t, 1H), 7.64 (s, 1H), 7.53 (d, 1H), 7.36-7.30 (m, 2H), 7.29-7.24 (m, 1H), 7.10 (d, 1H), 7.08 (dd, 1H), 7.05 (s, 1H), 5.96 (quin, 1H), 3.87 (s, 6H), 3.70 (br s, 2H), 3.30-3.26 (m, 2H), 3.22-3.17 (m, 5H), 3.06 (br s, 2H), 2.43 (s, 3H), 1.71 (d, 3H). LC-MS (method 7): m/z: [M+H]⁺=550, Rt=0.59 min.

### Example 284

### N-benzyl-2-[2-(5-{1-[(6,7-dimethoxy-2-methylquinazolin-4-yl)amino]ethyl}thiophen-2-yl)benzyl]glycinamide

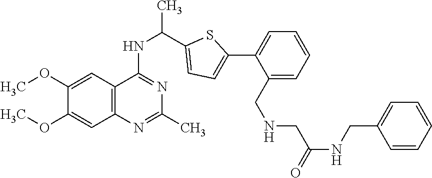 [SMILES: COc1cc2nc(C)nc(NC(C)c3ccc(-c4ccccc4CNCC(=O)NCc4ccccc4)s3)c2cc1OC]

A solution of 2-(5-{1-[(6,7-dimethoxy-2-methylquinazolin-4-yl)amino]ethyl}thiophen-2-yl)benzaldehyde (described in example 179a; 50.0 mg, 115 μmol) and N-benzylglycinamide hydrochloride (1:1) (23.1 mg, 115 μmol) in MeOH (1.2 mL) was stirred at room temperature during 5 hours. NaBH(OAc)₃ (48.9 mg, 231 μmol) was added and the solution stirred at room temperature during 15 mn. The reaction was quenched with aqueous NaOH (1.0 M, 1.0 mL), the mixture extracted with DCM and the solvent removed in vacuo. Purification by column chromatography (silica gel, MeOH/EtOAc 0-50% then MeOH) followed by preparative HPLC (basic conditions) gave the title compound as a white solid (27.3 mg, 40%). 1H-NMR (400 MHz, DMSO-d₆): δ [ppm]=8.26 (t, 1H), 8.15 (d, 1H), 7.64 (s, 1H), 7.55 (d, 1H), 7.33 (dd, 1H), 7.30 (s, 1H), 7.29-7.24 (m, 3H), 7.21-7.16 (m, 3H), 7.10 (d, 1H), 7.07-7.04 (m, 2H), 5.96 (quin, 1H), 4.26 (d, 2H), 3.87 (s, 3H), 3.86 (s, 3H), 3.74 (s, 2H), 3.14 (s, 2H), 2.43 (s, 3H), 1.70 (d, 3H). LC-MS (method 7): m/z: [M+H]⁺=582, Rt=0.68 min.

### Example 285

### 2-{[2-(5-{1-[(6,7-dimethoxy-2-methylquinazolin-4-yl)amino]ethyl}thiophen-2-yl)benzyl]amino}-1-(morpholin-4-yl)ethanone

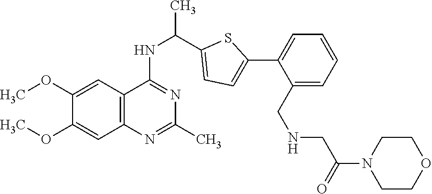 [SMILES: COc1cc2nc(C)nc(NC(C)c3ccc(-c4ccccc4CNCC(=O)N4CCOCC4)s3)c2cc1OC]

A solution of 2-(5-{1-[(6,7-dimethoxy-2-methylquinazolin-4-yl)amino]ethyl}thiophen-2-yl)benzaldehyde (described in example 179a; 50.0 mg, 115 μmol) and 2-amino-1-(morpholin-4-yl)ethanone hydrochloride (1:1) (20.8 mg, 115 μmol) in MeOH (1.2 mL) was stirred at room temperature during 5 hours. NaBH(OAc)₃ (48.9 mg, 231 μmol) was added and the solution stirred at room temperature during 15 mn. The reaction was quenched with aqueous NaOH (1.0 M, 1.0 mL), the mixture extracted with DCM and the solvent removed in vacuo. Purification by column chromatography (silica gel, MeOH/EtOAc 0-50% then MeOH) followed by preparative HPLC (basic conditions) gave the title compound as a white solid (32.4 mg, 50%). 1H-NMR (400 MHz, DMSO-d₆): δ [ppm]=8.15 (d, 1H), 7.65 (s, 1H), 7.54-7.49 (m, 1H), 7.35-7.30 (m, 2H), 7.29-7.24 (m, 1H), 7.15 (d, 1H), 7.08 (dd, 1H), 7.05 (s, 1H), 5.96 (quin, 1H), 3.87 (s, 6H), 3.74 (s, 2H), 3.54-3.48 (m, 4H), 3.43-3.37 (m, 2H), 3.33-3.29 (m, 4H), 2.43 (s, 3H), 1.71 (d, 3H). LC-MS (method 7): m/z: [M+H]⁺=562, Rt=0.58 min.

### Example 286

### 3-(5-{1-[(6,7-dimethoxy-2-methylquinazolin-4-yl)amino]ethyl}thiophen-2-yl)-1,5-dimethyl-1H-pyrrole-2-carbonitrile

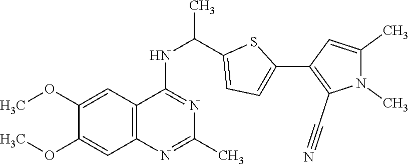

Under argon, N-[1-(5-bromothiophen-2-yl)ethyl]-6,7-dimethoxy-2-methylquinazolin-4-amine hydrochloride (1:1) (described in example 209; 100 mg, 225 μmol), 1,5-dimethyl-3-(4,4,5,5-tetramethyl-1,3,2-dioxaborolan-2-yl)-1H-pyrrole-2-carbonitrile (55.3 mg, 225 μmol), K₂SO₃ (124 mg, 899 μmol) and Pd(PPh₃)₄ (13.0 mg, 11.2 μmol) in dioxane (2.5 mL) and H₂O (500 μL) were stirred at 110° C. during 8 hours. H₂O was added, the mixture extracted with DCM and the solvent removed in vacuo. Purification by preparative HPLC (basic conditions) gave the title compound as a white solid (87.5 mg, 87%). 1H-NMR (400 MHz, DMSO-d₆): δ [ppm]=8.11 (d, 1H), 7.63 (s, 1H), 7.19 (d, 1H), 7.06-7.04 (m, 2H), 6.22 (d, 1H), 5.89 (quin, 1H), 3.87 (s, 6H), 3.58 (s, 3H), 2.43 (s, 3H), 2.21 (s, 3H), 1.69 (d, 3H). LC-MS (method 7): m/z: [M+H]⁺=448, Rt=0.90 min.

### Example 287

### 5-{1-[(6,7-dimethoxy-2-methylquinazolin-4-yl)amino]ethyl}-2,3′-bithiophene-4′-carbonitrile

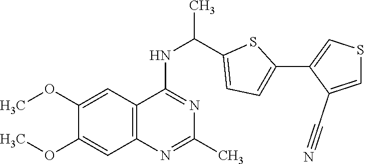 [SMILES: COc1cc2nc(C)nc(NC(C)c3ccc(-c4cscc4C#N)s3)c2cc1OC]

Under argon, N-[1-(5-bromothiophen-2-yl)ethyl]-6,7-dimethoxy-2-methylquinazolin-4-amine hydrochloride (1:1) (described in example 209; 100 mg, 225 μmol), 4-(4,4,5,5-tetramethyl-1,3,2-dioxaborolan-2-yl)thiophene-3-carbonitrile (52.9 mg, 225 μmol), K₂CO₃ (124 mg, 899 μmol) and Pd(PPh₃)₄ (13.0 mg, 11.2 μmol) in dioxane (2.3 mL) and H₂O (460 μL) were stirred at 110° C. during 8 hours. H₂O was added, the mixture extracted with DCM and the solvent removed in vacuo. Purification by preparative HPLC (basic conditions) gave the title compound as a white solid (69.5 mg, 71%). 1H-NMR (400 MHz, DMSO-d₆): δ [ppm]=8.68 (d, 1H), 8.15 (d, 1H), 7.86 (d, 1H), 7.64 (s, 1H), 7.35 (d, 1H), 7.14 (dd, 1H), 7.05 (s, 1H), 5.92 (quin, 1H), 3.87 (s, 6H), 2.44 (s, 3H), 1.71 (d, 3H). LC-MS (method 7): m/z: [M+H]⁺=437, Rt=0.87 min.

### Example 288

### N-[1-(5-{2-[(diethylamino)methyl]phenyl}thiophen-2-yl)ethyl]-6,7-dimethoxy-2-methylquinazolin-4-amine

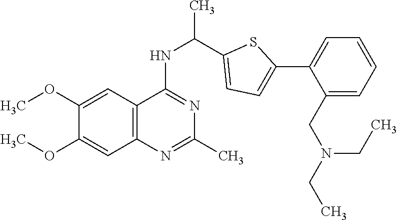 [SMILES: CCN(CC)Cc1ccccc1-c1ccc(C(C)Nc2nc(C)nc3cc(OC)c(OC)cc23)s1]

To 2-(5-{1-[(6,7-dimethoxy-2-methylquinazolin-4-yl)amino]ethyl}thiophen-2-yl)benzaldehyde (described in example 179a; 20.0 mg, 46.1 μmol), N-ethylethanamine (5.8 μL, 55 μmol) and acetic acid (5.3 μL, 92 μmol) in 1,2-dichloroethane (500 μL) was added NaBH(OAc)₃ (19.6 mg, 92.3 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a white solid (5.10 mg, 22%). 1H-NMR (400 MHz, DMSO-d₆): δ [ppm]=8.14 (d, 1H), 7.64 (s, 1H), 7.57-7.51 (m, 1H), 7.33-7.28 (m, 2H), 7.27-7.22 (m, 1H), 7.09 (d, 1H), 7.06 (dd, 1H), 7.04 (s, 1H), 5.95 (quin, 1H), 3.87 (s, 6H), 3.54 (s, 2H), 2.42 (s, 3H), 2.39 (q, 4H), 1.71 (d, 3H), 0.86 (t, 6H). LC-MS (method 7): m/z: [M+H]⁺=491, Rt=0.59 min.

### Example 289

### 2-[2-(5-{1-[(6,7-dimethoxy-2-methylquinazolin-4-yl)amino]ethyl}thiophen-2-yl)benzyl]-N-phenylglycinamide

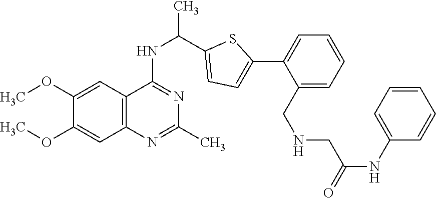 [SMILES: COc1cc2nc(C)nc(NC(C)c3ccc(-c4ccccc4CNCC(=O)Nc4ccccc4)s3)c2cc1OC]

A solution of 2-(5-{1-[(6,7-dimethoxy-2-methylquinazolin-4-yl)amino]ethyl}thiophen-2-yl)benzaldehyde (described in example 179a; 50.0 mg, 115 μmol) and N-phenylglycinamide hydrochloride (1:1) (21.5 mg, 115 μmol) in MeOH (1.2 mL) was stirred at room temperature during 5 hours. NaBH(OAc)₃ (48.9 mg, 231 μmol) was added and the solution stirred at room temperature during 15 mn. The reaction was quenched with aqueous NaOH (1.0 M, 1.0 mL), the mixture extracted with DCM and the solvent removed in vacuo. Purification by column chromatography (silica gel, MeOH/EtOAc 0-50% then MeOH) followed by preparative HPLC (basic conditions) gave the title compound as an off-white solid (1.40 mg, 2%). 1H-NMR (400 MHz, DMSO-d₆): δ [ppm]=9.91 (br s, 1H), 7.68 (s, 1H), 7.61 (br d, 1H), 7.55 (br d, 2H), 7.43 7.24 (m, 6H), 7.14 (d, 1H), 7.09-7.02 (m, 3H), 5.95 (quin, 1H), 4.05-3.90 (m, 2H), 3.88 (s, 3H), 3.87 (s, 3H), 2.46 (s, 3H), 1.69 (d, 3H). LC-MS (method 7): m/z: [M+H]⁺=568, Rt=0.67 min.

### Example 290

### 1-[2-(5-{1-[(6,7-dimethoxy-2-methylquinazolin-4-yl)amino]ethyl}thiophen-2-yl)benzyl]piperidine-3-carboxamide

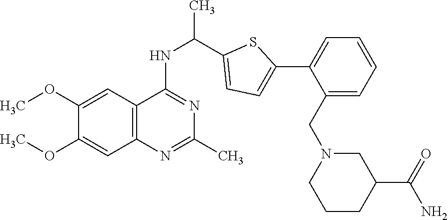 [SMILES: COc1cc2nc(C)nc(NC(C)c3ccc(-c4ccccc4CN4CCCC(C(N)=O)C4)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), piperidine-3-carboxamide (29.6 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature during 5 hours. The reaction was quenched with aqueous NaOH (1.0 M, 1.0 mL), the mixture extracted with DCM and the solvent removed in vacuo. Purification by preparative HPLC (basic conditions) gave the title compound as a white solid (8.90 mg, 14%). 1H-NMR (400 MHz, DMSO-d₆): δ [ppm]=8.15 (dd, 1H), 7.65 (s, 1H), 7.41 (dt, 1H), 7.38-7.34 (m, 1H), 7.33-7.27 (m, 2H), 7.21 (br s, 1H), 7.19 (t, 1H), 7.08 (d, 1H), 7.05 (s, 1H), 6.72 (br s, 1H), 5.97 (br t, 1H), 3.87 (s, 6H), 3.47 (dd, 1H), 2.75 (br d, 1H), 2.43 (s, 3H), 2.28-2.18 (m, 1H), 1.96 (br t, 1H), 1.92-1.82 (m, 1H), 1.71 (d, 3H), 1.69-1.64 (m, 1H), 1.59-1.46 (m, 1H), 1.41-1.25 (m, 2H). LC-MS (method 7): m/z: [M+H]⁺=546, Rt=0.57 min.

### Example 291

### N-{1-[5-(2-{[(2,2-difluoroethyl)(methyl)amino]methyl}phenyl)thiophen-2-yl]ethyl}-6,7-dimethoxy-2-methylquinazolin-4-amine

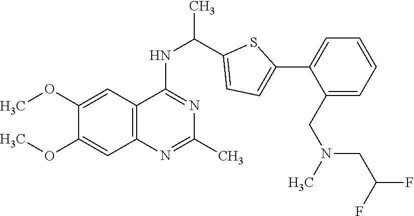 [SMILES: COc1cc2nc(C)nc(NC(C)c3ccc(-c4ccccc4CN(C)CC(F)F)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), 2,2-difluoro-N-methylethanamine (21.9 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a white solid (29.6 mg, 50%). 1H-NMR (400 MHz, DMSO-d₆): δ [ppm]=8.14 (d, 1H), 7.65 (s, 1H), 7.51 (d, 1H), 7.36-7.26 (m, 3H), 7.13-7.11 (m, 1H), 7.08 (dd, 1H), 7.05 (s, 1H), 5.96 (quin, 1H), 6.05 (tt, 1H), 3.87 (s, 6H), 3.63 (s, 2H), 2.74 (td, 2H), 2.43 (s, 3H), 2.19 (s, 3H), 1.71 (d, 3H). LC-MS (method 7): m/z: [M+H]⁺=513, Rt=0.94 min.

### Example 292

### N-[1-{5-[2-(aminomethyl)-5-chlorophenyl]thiophen-2-yl}ethyl]-6,7-dimethoxy-2-methylquinazolin-4-amine

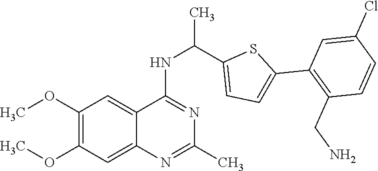 [SMILES: COc1cc2nc(C)nc(NC(C)c3ccc(-c4cc(Cl)ccc4CN)s3)c2cc1OC]

### Step a

### 1-(2-bromo-4-chlorophenyl)methanamine

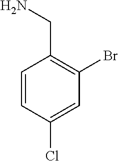

Borane tetrahydrofuran complex (55 mL, 1.0 M in THF) were provided with ice cooling, then slowly a solution of 2-bromo-4-chlorobenzonitrile (4.00 g, 18.5 mmol) in THF (10 mL) was added. Thereafter the reaction-mixture was heated under reflux for 16 hours. The reaction was quenched by addition of methanol. 20 mL of hydrochloric acid (1.0 N) were added dropwise with ice cooling. The solution was rendered alkaline with 1.0 M sodium hydroxide solution and extracted with dichloromethane. The organic phase was dried over sodium sulfate and evaporated to yield 2.10 g (36%) of the title compound as the off-white solid. The reaction was monitored by TLC (ethyl acetate/petroleum ether=1:1, Rf=0.3). MS (ESIpos): m/z=220 [M+H]⁺.

### Step b

### tert-butyl (2-bromo-4-chlorobenzyl)carbamate

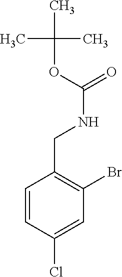 [SMILES: CC(C)(C)OC(=O)NCc1ccc(Cl)cc1Br]

1-(2-bromo-4-chlorophenyl)methanamine (1.60 g, 7.3 mmol), di-tert-butyl dicarbonate (2.06 g, 9.4 mmol), and triethylamine (1.84 g, 18.1 mmol), were added into 25 mL of dichloromethane. The resulting mixture was stirred at room temperature for four hours. The resulting mixture was washed with water and the combined organic layers were concentrated on a rotary evaporator.

The reaction was purified by silica gel chromatography column (EA/PE=1:19, Rf=0.2) to give 1.90 g (74%) of the title compound as a light yellow oil. MS (ESIpos): m/z=319 [M+H]⁺.

### Step c

### tert-butyl [4-chloro-2-(4,4,5,5-tetramethyl-1,3,2-dioxaborolan-2-yl)benzyl]carbamate

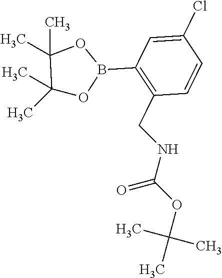 [SMILES: CC(C)(C)OC(=O)NCc1ccc(Cl)cc1B1OC(C)(C)C(C)(C)O1]

tert-butyl (2-bromo-4-chlorobenzyl)carbamate (1.0 g, 2.8 mmol), 4,4,4′,4′,5,5,5′,5′-octamethyl-2,2′-bi(1,3,2-dioxaborolane) (1.78 g, 7.0 mmol), potassium acetate (0.83 g, 8.4 mmol) and 1,1′-bis(diphenylphosphino)ferrocenepalladiumdichloride CH₂Cl₁₋₂ (0.23 g, 0.3 mmol), were added in 15 mL of 1,4-dioxane under N₂ atmosphere. The resulting mixture was stirred at 100° C. for 15 h. The solvent was removed in vacuo and was diluted by addition of water. The mixture was extracted with ethyl acetate, was washed with water and the combine organic layers was concentrated under vacuum. The residue was purified by silica gel column on chromatography (ethyl acetate/petroleum ether=3:7) to 500 mg (36%) of the product as a light yellow oil. MS (ESIpos): m/z=368 [M+H]⁺.

### Step d

### tert-butyl [4-chloro-2-(5-{1-[(6,7-dimethoxy-2-methylquinazolin-4-yl)amino]ethyl}-2-thienyl)benzyl]carbamate

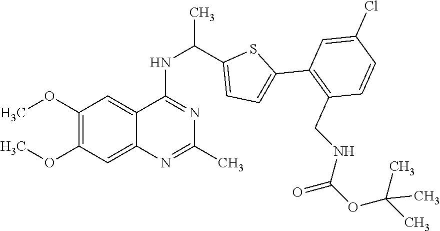 [SMILES: COc1cc2nc(C)nc(NC(C)c3ccc(-c4cc(Cl)ccc4CNC(=O)OC(C)(C)C)s3)c2cc1OC]

N-[1-(5-bromo-2-thienyl)ethyl]-6,7-dimethoxy-2-methylquinazolin-4-amine (described in example 209; 150 mg, 0.3 mmol) was dissolved in 1,4-dioxane (10 mL) and water (2 mL), then tert-butyl [4-chloro-2-(4,4,5,5-tetramethyl-1,3,2-dioxaborolan-2-yl)benzyl]carbamate (430 mg, 0.9 mmol), potassium carbonate (199 mg, 1.4 mmol) and tetrakis(triphenylphosphine)palladium(0) (36 mg, 0.03 mmol) were added under N₂ atmosphere. The reaction was stirred at 100° C. for 16 h. The solvent was removed in vacuo and was diluted by addition of water. The mixture was extracted with dichloromethane, was washed with water and the combined organic layers were concentrated under vacuum. The residue was purified by silica gel column on chromatography (dichloromethane:methanol=13:1, Rf=0.3) to yield 150 mg (63%) of the title compound as a light yellow solid. MS (ESIpos): m/z=569 [M+H]⁺.

### Step e

### N-[1-{5-[2-(aminomethyl)-5-chlorophenyl]thiophen-2-yl}ethyl]-6,7-dimethoxy-2-methylquinazolin-4-amine

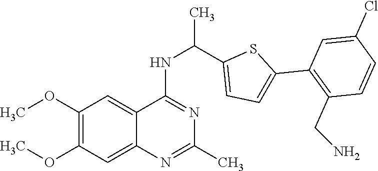 [SMILES: COc1cc2nc(C)nc(NC(C)c3ccc(-c4cc(Cl)ccc4CN)s3)c2cc1OC]

tert-butyl [4-chloro-2-(5-{1-[(6,7-dimethoxy-2-methylquinazolin-4-yl)amino]ethyl}-2-thienyl)benzyl]carbamate, (100 mg, 0.14 mmol), was dissolved in 1,4-dioxane (3 mL), then hydrochloric acid (4.0 M, 3 mL) was added at 0° C. The resulting mixture was stirred at room temperature for 3 hours. By addition of saturated sodium carbonate solution the pH value was adjusted to pH=7. The water layer was extracted with ethyl acetate, washed with water and the combined organic layers were concentrated under vacuum. The residue was purified by preparative HPLC (Column: XBridge Prep C18 OBD Column 19×150 mm 5 μm; Mobile Phase A: Water (10 mM NH₄HCO₃), Mobile Phase B: Acetonitrile; Flow rate: 20 mL/min; Gradient: 25% B to 50% B in 7 min; Detector: 254 nm, 220 nm) to give 17.9 mg (25%) of the title compound as an off-white solid. MS (ESIpos): m/z=469 [M+H]⁺. ¹H-NMR (400 MHz, DMSO-d₆): δ [ppm]=8.24 (s, 2H), 8.16 (d, 1H), 7.66 (s, 1H), 7.62 (d, 1H), 7.47 (m, 1H), 7.36 (m, 1H), 7.19-7.11 (m, 2H), 7.06 (s, 1H), 6.03-5.90 (m, 1H), 3.88 (m, 6H), 2.44 (s, 3H), 1.73 (d, 3H).

### Example 293

### N-[2-(5-{1-[(6,7-dimethoxy-2-methylquinazolin-4-yl)amino]ethyl}thiophen-2-yl)-5-fluorobenzyl]-1H-imidazole-2-carboxamide

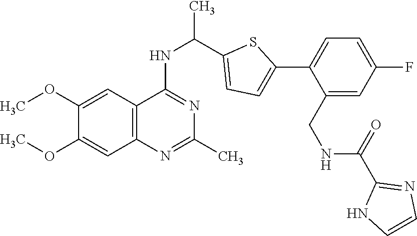 [SMILES: COc1cc2nc(C)nc(NC(C)c3ccc(-c4ccc(F)cc4CNC(=O)c4ncc[nH]4)s3)c2cc1OC]

A solution of N-[1-{5-[2-(aminomethyl)-4-fluorophenyl]thiophen-2-yl}ethyl]-6,7-dimethoxy-2-methylquinazolin-4-amine (described in example 234; 50.0 mg, 110 μmol), 1H-imidazole-2-carboxylic acid (12.4 mg, 110 μmol), PyBOP (115 mg, 221 μmol) and N,N-diisopropylethylamine (95 μL, 550 μmol) in DMF (1.0 mL) was stirred at room temperature overnight. Purification by preparative HPLC (basic conditions) gave the title compound as a white solid (24.1 mg, 39%). 1H-NMR (400 MHz, DMSO-d₆): δ [ppm]=13.02 (br s, 1H), 9.04 (t, 1H), 8.17 (d, 1H), 7.65 (s, 1H), 7.37 (dd, 1H), 7.29 (br s, 1H), 7.14-7.06 (m, 5H), 7.05 (s, 1H), 5.97 (quin, 1H), 4.52 (d, 2H), 3.87 (s, 6H), 2.44 (s, 3H), 1.72 (d, 3H). LC-MS (method 7): m/z: [M+H]⁺=547, Rt=0.83 min.

### Example 294

### N-[2-(5-{1-[(6,7-dimethoxy-2-methylquinazolin-4-yl)amino]ethyl}thiophen-2-yl)-5-fluorobenzyl]-1H-imidazole-5-carboxamide

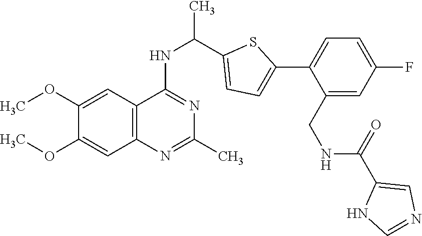 [SMILES: COc1cc2nc(C)nc(NC(C)c3ccc(-c4ccc(F)cc4CNC(=O)c4cnc[nH]4)s3)c2cc1OC]

A solution of N-[1-{5-[2-(aminomethyl)-4-fluorophenyl]thiophen-2-yl}ethyl]-6,7-dimethoxy-2-methylquinazolin-4-amine (described in example 234; 50.0 mg, 110 μmol), 1H-imidazole-5-carboxylic acid (12.4 mg, 110 μmol), PyBOP (115 mg, 221 μmol) and N,N-diisopropylethylamine (95 μL, 550 μmol) in DMF (1.0 mL) was stirred at room temperature overnight. Purification by preparative HPLC (basic conditions) gave the title compound as a white solid (22.8 mg, 37%). 1H-NMR (400 MHz, DMSO-d₆): δ [ppm]=12.52 (br s, 1H), 8.55 (br t, 1H), 8.17 (d, 1H), 7.74 (d, 1H), 7.65 (s, 1H), 7.63 (d, 1H), 7.36 (dd, 1H), 7.13-7.05 (m, 4H), 7.05 (s, 1H), 5.97 (quin, 1H), 4.50 (s, 1H), 4.49 (s, 1H), 3.87 (s, 6H), 2.43 (s, 3H), 1.72 (d, 3H). LC-MS (method 7): m/z: [M+H]⁺=547, Rt=0.77 min.

### Example 295

### N-[2-(5-{1-[(6,7-dimethoxy-2-methylquinazolin-4-yl)amino]ethyl}thiophen-2-yl)-5-fluorobenzyl]-N&lt;sup&gt;2&lt;/sup&gt;-(2,2,2-trifluoroethyl)glycinamide

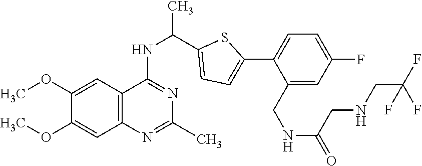 [SMILES: COc1cc2nc(C)nc(NC(C)c3ccc(-c4ccc(F)cc4CNC(=O)CNCC(F)(F)F)s3)c2cc1OC]

A solution of N-[1-{5-[2-(aminomethyl)-4-fluorophenyl]thiophen-2-yl}ethyl]-6,7-dimethoxy-2-methylquinazolin-4-amine (described in example 234; 50.0 mg, 110 μmol), N-(2,2,2-trifluoroethyl)glycine (17.4 mg, 110 μmol), PyBOP (115 mg, 221 μmol) and N,N-diisopropylethylamine (95 μL, 550 μmol) in DMF (1.0 mL) was stirred at room temperature overnight. Purification by preparative HPLC (basic conditions) gave the title compound as a white solid (35.0 mg, 50%). 1H-NMR (400 MHz, DMSO-d₆): δ [ppm]=8.31 (t, 1H), 8.15 (d, 1H), 7.64 (s, 1H), 7.36 (dd, 1H), 7.17-7.11 (m, 2H), 7.09 (dd, 1H), 7.05 (s, 1H), 7.03 (d, 1H), 5.96 (quin, 1H), 4.36 (d, 2H), 3.87 (s, 6H), 3.28 (d, 2H), 2.96 (dt, 1H), 2.85 (quin, 1H), 2.43 (s, 3H), 1.71 (d, 3H). LC-MS (method 7): m/z: [M+H]⁺=592, Rt=0.92 min.

### Example 296

### N-[2-(5-{1-[(6,7-dimethoxy-2-methylquinazolin-4-yl)amino]ethyl}thiophen-2-yl)-5-fluorobenzyl]-1H-indole-2-carboxamide

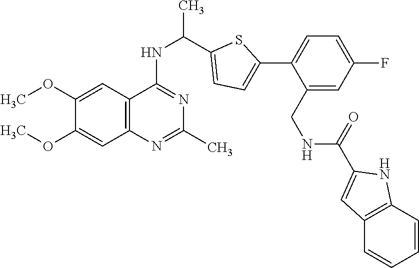 [SMILES: COc1cc2nc(C)nc(NC(C)c3ccc(-c4ccc(F)cc4CNC(=O)c4cc5ccccc5[nH]4)s3)c2cc1OC]

A solution of N-[1-{5-[2-(aminomethyl)-4-fluorophenyl]thiophen-2-yl}ethyl]-6,7-dimethoxy-2-methylquinazolin-4-amine (described in example 234; 50.0 mg, 110 μmol), 1H-indole-2-carboxylic acid (17.8 mg, 110 μmol), PyBOP (115 mg, 221 μmol) and N,N-diisopropylethylamine (95 μL, 550 μmol) in DMF (1.0 mL) was stirred at room temperature overnight. Purification by preparative HPLC (basic conditions) gave the title compound as a white solid (32.8 mg, 49%). 1H-NMR (400 MHz, DMSO-d₆): δ [ppm]=11.64-11.57 (m, 1H), 9.02 (t, 1H), 8.17 (d, 1H), 7.65 (s, 1H), 7.62 (d, 1H), 7.44-7.38 (m, 2H), 7.21-7.18 (m, 2H), 7.18 7.15 (m, 1H), 7.15-7.11 (m, 3H), 7.08-7.01 (m, 2H), 5.97 (quin, 1H), 4.58 (d, 2H), 3.87 (s, 6H), 2.43 (s, 3H), 1.73 (d, 3H). LC-MS (method 7): m/z: [M+H]⁺=596, Rt=0.99 min.

### Example 297

### 2-{[2-(5-{1-[(6,7-dimethoxy-2-methylquinazolin-4-yl)amino]ethyl}thiophen-2-yl)benzyl]amino}ethanol

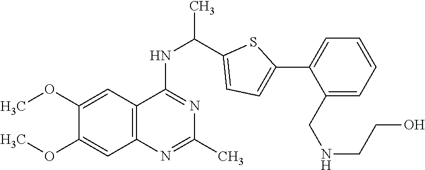 [SMILES: COc1cc2nc(C)nc(NC(C)c3ccc(-c4ccccc4CNCCO)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), 2-aminoethanol (14 μL, 230 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a white solid (12.5 mg, 23%). 1H-NMR (400 MHz, DMSO-d₆): δ [ppm]=8.15 (d, 1H), 7.65 (s, 1H), 7.52-7.48 (m, 1H), 7.34-7.28 (m, 2H), 7.28-7.23 (m, 1H), 7.16 (d, 1H), 7.08 (d, 1H), 7.05 (s, 1H), 5.96 (quin, 1H), 4.45 (t, 1H), 3.87 (s, 6H), 3.72 (s, 2H), 3.41 (q, 2H), 2.56-2.53 (m, 2H), 2.43 (s, 3H), 1.71 (d, 3H). LC-MS (method 7): m/z: [M+H]⁺=479, Rt=0.55 min.

### Example 298

### 2-{[2-(5-{1-[(6,7-dimethoxy-2-methylquinazolin-4-yl)amino]ethyl}thiophen-2-yl)benzyl](methyl)amino}ethanol

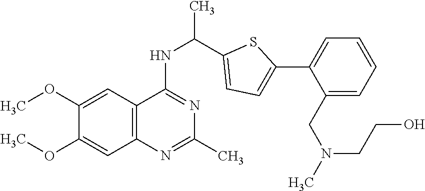 [SMILES: COc1cc2nc(C)nc(NC(C)c3ccc(-c4ccccc4CN(C)CCO)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), 2-(methylamino)ethanol (18 μL, 230 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a white solid (31.6 mg, 56%). 1H-NMR (400 MHz, DMSO-d₆): δ [ppm]=8.15 (d, 1H), 7.65 (s, 1H), 7.51 (dd, 1H), 7.35-7.29 (m, 2H), 7.29-7.24 (m, 1H), 7.18 (d, 1H), 7.07 (dd, 1H), 7.05 (s, 1H), 5.96 (quin, 1H), 4.35 (t, 1H), 3.87 (s, 6H), 3.52-3.48 (m, 2H), 3.48-3.42 (m, 2H), 2.43 (s, 3H), 2.40 (t, 2H), 2.10 (s, 3H), 1.71 (d, 3H). LC-MS (method 7): m/z: [M+H]⁺=493, Rt=0.56 min.

### Example 299

### N-[2-(5-{1-[(6,7-dimethoxy-2-methylquinazolin-4-yl)amino]ethyl}thiophen-2-yl)-5-fluorobenzyl]-N²-phenylglycinamide

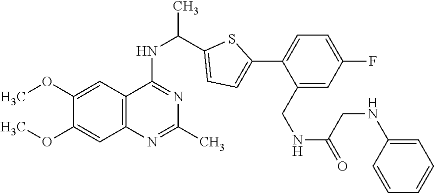 [SMILES: COc1cc2nc(C)nc(NC(C)c3ccc(-c4ccc(F)cc4CNC(=O)CNc4ccccc4)s3)c2cc1OC]

A solution of N-[1-{5-[2-(aminomethyl)-4-fluorophenyl]thiophen-2-yl}ethyl]-6,7-dimethoxy-2-methylquinazolin-4-amine (described in example 234; 20.0 mg, 44.2 μmol), N-phenylglycine (13.4 mg, 88.4 μmol), PyBOP (46.0 mg, 88.4 μmol) and N,N-diisopropylethylamine (38 μL, 220 μmol) in DMF (1.0 mL) was stirred at room temperature overnight. Purification by preparative HPLC (basic conditions) gave the title compound as a white solid (3.50 mg, 14%). 1H-NMR (400 MHz, DMSO-d₆): δ [ppm]=8.59 (t, 1H), 8.14 (d, 1H), 7.63 (s, 1H), 7.42-7.38 (m, 2H), 7.35-7.27 (m, 3H), 7.26-7.21 (m, 1H), 7.10-7.03 (m, 3H), 6.97 (dd, 1H), 6.95 (dt, 1H), 5.94 (quin, 1H), 4.40 (s, 1H), 4.34-4.29 (m, 2H), 3.87 (s, 3H), 3.86-3.85 (m, 3H), 2.42 (s, 3H), 1.70 (d, 3H). LC-MS (method 7): m/z: [M+H]⁺=586, Rt=0.69 min.

### Example 300

### 6,7-di methoxy-2-methyl-N-{1-[5-(2-{[(2,2,2-trifluoroethyl)amino]methyl}phenyl)thiophen-2-yl]ethyl}quinazolin-4-amine

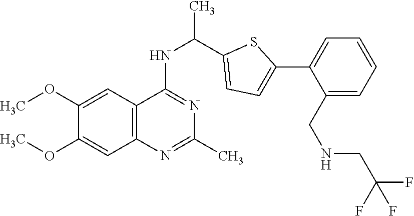 [SMILES: COc1cc2nc(C)nc(NC(C)c3ccc(-c4ccccc4CNCC(F)(F)F)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), 2,2,2-trifluoroethanamine (18 μL, 230 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a white solid (26.9 mg, 45%). 1H-NMR (400 MHz, DMSO-d₆): δ [ppm]=8.14 (d, 1H), 7.65 (s, 1H), 7.53 (d, 1H), 7.36-7.25 (m, 3H), 7.13 (d, 1H), 7.08 (d, 1H), 7.05 (s, 1H), 5.96 (quin, 1H), 3.87 (s, 6H), 3.83 (d, 2H), 3.20 (qd, 2H), 2.76 (quin, 1H), 2.43 (s, 3H), 1.71 (d, 3H). LC-MS (method 7): m/z: [M+H]⁺=517, Rt=0.98 min.

### Example 301

### 6,7-di methoxy-2-methyl-N-[1-(5-{2-[(pyridin-2-ylamino)methyl]phenyl}thiophen-2-yl)ethyl]quinazolin-4-amine

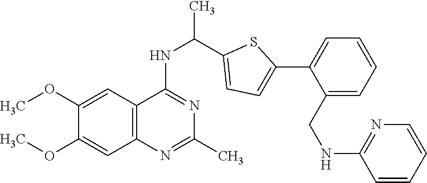 [SMILES: COc1cc2nc(C)nc(NC(C)c3ccc(-c4ccccc4CNc4ccccn4)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), pyridin-2-amine (21.7 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a white solid (15.5 mg, 26%). 1H-NMR (400 MHz, DMSO-d₆): δ [ppm]=8.15 (d, 1H), 7.89 (ddd, 1H), 7.64 (s, 1H), 7.42 (dd, 1H), 7.36-7.23 (m, 4H), 7.09 (s, 2H), 7.05 (s, 1H), 6.93 (t, 1H), 6.49-6.43 (m, 2H), 5.96 (quin, 1H), 4.51 (d, 2H), 3.87 (s, 3H), 3.86 (s, 3H), 2.43 (s, 3H), 1.71 (d, 3H). LC-MS (method 7): m/z: [M+H]⁺=512, Rt=0.65 min.

### Example 302

### 6,7-dimethoxy-2-methyl-N-[1-(5-{2-[(1H-pyrazol-3-ylamino)methyl]phenyl}thiophen-2-yl)ethyl]quinazolin-4-amine

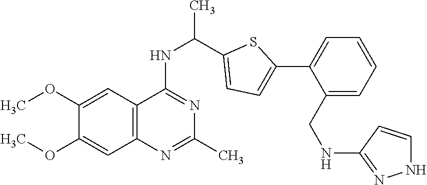 [SMILES: COc1cc2nc(C)nc(NC(C)c3ccc(-c4ccccc4CNc4cc[nH]n4)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), 1H-pyrazol-3-amine (19.2 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a white solid (17.0 mg, 28%). 1H-NMR (400 MHz, DMSO-d₆): δ [ppm]=11.52-11.32 (m, 1H), 8.16 (d, 1H), 7.64 (s, 1H), 7.52 (d, 1H), 7.34-7.27 (m, 3H), 7.26-7.21 (m, 1H), 7.10-7.07 (m, 2H), 7.05 (s, 1H), 5.96 (quin, 1H), 5.52 (br s, 1H), 5.39 (br s, 1H), 4.28 (br d, 2H), 3.87 (s, 3H), 3.86 (s, 3H), 2.43 (s, 3H), 1.71 (d, 3H). LC-MS (method 7): m/z: [M+H]⁺=501, Rt=0.79 min.

### Example 303

### 1-(3,4-dihydroisoquinolin-2(1H)-yl)-2-{[2-(5-{1-[(6,7-dimethoxy-2-methylquinazolin-4-yl)amino]ethyl}thiophen-2-yl)benzyl]amino}ethanone

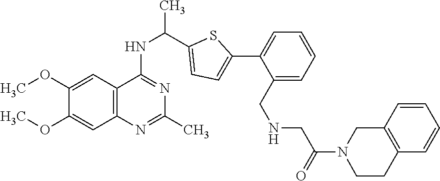 [SMILES: COc1cc2nc(C)nc(NC(C)c3ccc(-c4ccccc4CNCC(=O)N4CCc5ccccc5C4)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), 2-amino-1-(3,4-dihydroisoquinolin-2(1H)-yl)ethanone hydrochloride (1:1) (52.3 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature during 5 hours. The reaction was quenched with aqueous NaOH (1.0 M, 1.0 mL), the mixture extracted with DCM and the solvent removed in vacuo. Purification by preparative HPLC (basic conditions) gave the title compound as an off-white solid (28.5 mg, 41%). 1H-NMR (400 MHz, DMSO-d₆): δ [ppm]=8.14 (d, 1H), 7.64 (s, 1H), 7.52 (t, 1H), 7.34 7.24 (m, 3H), 7.19-7.11 (m, 5H), 7.05-7.02 (m, 2H), 5.95 (br quin, 1H), 4.58 (s, 1H), 4.54 (s, 1H), 3.86 (s, 6H), 3.76 (s, 2H), 3.64 (t, 1H), 3.55 (t, 1H), 3.44 (s, 2H), 2.79 (t, 1H), 2.73 (t, 1H), 2.43 (s, 3H), 1.70 (br d, 3H). LC-MS (method 7): m/z: [M+H]⁺=608, Rt=0.69 min.

### Example 304

### N-[1-{5-[4-fluoro-2-({[(1-methyl-1H-imidazol-2-yl)methyl]amino}methyl)phenyl]thiophen-2-yl}ethyl]-6,7-dimethoxy-2-methylquinazolin-4-amine

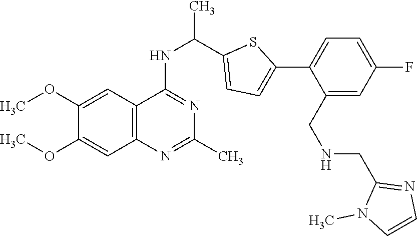 [SMILES: COc1cc2nc(C)nc(NC(C)c3ccc(-c4ccc(F)cc4CNCc4nccn4C)s3)c2cc1OC]

To N-[1-{5-[2-(aminomethyl)-4-fluorophenyl]thiophen-2-yl}ethyl]-6,7-dimethoxy-2-methylquinazolin-4-amine (described in example 234; 50.0 mg, 110 μmol), 1-methyl-1H-imidazole-2-carbaldehyde (12.2 mg, 110 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a light yellow solid (4.90 mg, 8%). 1H-NMR (400 MHz, DMSO-d₆): δ [ppm]=8.15 (d, 1H), 7.64 (s, 1H), 7.39 (dd, 1H), 7.34 (dd, 1H), 7.09 (td, 1H), 7.06-7.03 (m, 2H), 7.01 (d, 1H), 6.98 (dd, 1H), 6.71 (dd, 1H), 5.94 (quin, 1H), 3.87 (s, 3H), 3.86 (s, 3H), 3.70 (br d, 3H), 3.55 (s, 3H), 3.20 (s, 1H), 2.43 (s, 3H), 1.70 (d, 3H). LC-MS (method 7): m/z: [M+H]⁺=547, Rt=0.64 min.

### Example 305

### 6,7-dimethoxy-2-methyl-N-[1-{5-[2-(piperazin-1-ylmethyl)phenyl]thiophen-2-yl}ethyl]quinazolin-4-amine hydrochloride

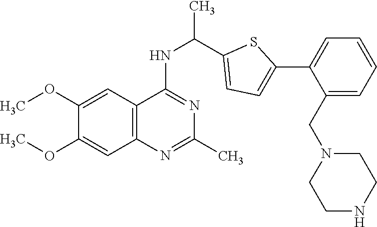 [SMILES: COc1cc2nc(C)nc(NC(C)c3ccc(-c4ccccc4CN4CCNCC4)s3)c2cc1OC]

To tert-butyl 4-[2-(5-{1-[(6,7-dimethoxy-2-methylquinazolin-4-yl)amino]ethyl}thiophen-2-yl)benzyl]piperazine-1-carboxylate (described in example 306; 70.0 mg, 116 μmol) in MeOH (1.1 mL) and H₂O (200 μL) was added dropwise acetyl chloride (100 μL, 1.4 mmol). The solution was stirred at room temperature overnight and the solvent then removed in vacuo. The residue was stirred in Et₂O then filtered to give the title compound as a yellow solid (57.6 mg, 85%). NMR is broad. Selected peaks: ¹H-NMR (400 MHz, DMSO-d₆): δ [ppm]=14.48 (br s, 1H), 10.08 (br d, 1H), 8.07 (s, 1H), 7.21 (s, 1H), 7.20 (s, 2H), 6.11 (quin, 1H), 3.96 (s, 3H), 3.95 (s, 3H), 2.66 (s, 3H), 1.81 (d, 3H). LC-MS (method 7): m/z: [M+H]⁺=504, Rt=0.63 min.

### Example 306

### tert-butyl 4-[2-(5-{1-[(6,7-dimethoxy-2-methylquinazolin-4-yl)amino]ethyl}thiophen-2-yl)benzyl]piperazine-1-carboxylate

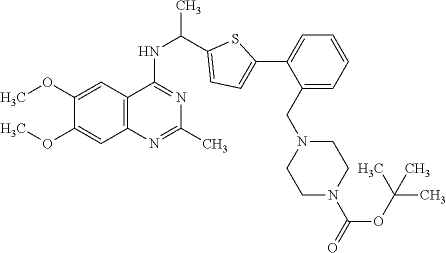 [SMILES: COc1cc2nc(C)nc(NC(C)c3ccc(-c4ccccc4CN4CCN(C(=O)OC(C)(C)C)CC4)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), tert-butyl piperazine-1-carboxylate (43.0 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification of 30.0 mg of the crude residue by preparative HPLC (basic conditions) gave the title compound as an off-white solid (11.4 mg, 16%). 1H-NMR (400 MHz, DMSO-d₆): δ [ppm]=8.15 (d, 1H), 7.65 (s, 1H), 7.43-7.35 (m, 2H), 7.33-7.27 (m, 2H), 7.17 (d, 1H), 7.07 (dd, 1H), 7.04 (s, 1H), 5.96 (quin, 1H), 3.87 (s, 6H), 3.42 (s, 2H), 3.19 (br s, 4H), 2.42 (s, 3H), 2.26 (br s, 4H), 1.71 (d, 3H), 1.37 (s, 9H). LC-MS (method 7): m/z: [M+H]⁺=604, Rt=0.79 min.

### Example 307

### N-[2-(5-{1-[(6,7-dimethoxy-2-methylquinazolin-4-yl)amino]ethyl}thiophen-2-yl)-5-fluorobenzyl]acetamide

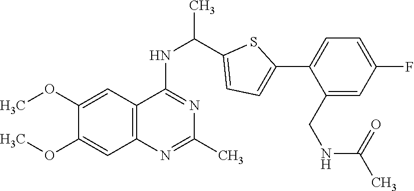 [SMILES: COc1cc2nc(C)nc(NC(C)c3ccc(-c4ccc(F)cc4CNC(C)=O)s3)c2cc1OC]

A solution of N-[1-{5-[2-(aminomethyl)-4-fluorophenyl]thiophen-2-yl}ethyl]-6,7-dimethoxy-2-methylquinazolin-4-amine (described in example 234; 100 mg, 221 μmol), acetic acid (19 μL, 330 μmol), PyBOP (230 mg, 442 μmol) and N,N-diisopropylethylamine (190 μL, 1.1 mmol) in DMF (1.0 mL) was stirred at room temperature overnight. Purification by preparative HPLC (basic conditions) gave the title compound as an off-white solid (66.5 mg, 57%). 1H-NMR (400 MHz, DMSO-d₆): δ [ppm]=8.34 (t, 1H), 8.16 (d, 1H), 7.64 (s, 1H), 7.36 (dd, 1H), 7.16-7.11 (m, 2H), 7.09 (dd, 1H), 7.05 (s, 1H), 7.02 (d, 1H), 5.95 (quin, 1H), 4.30 (d, 2H), 3.87 (s, 6H), 2.43 (s, 3H), 1.87 (s, 3H), 1.71 (d, 3H). LC-MS (method 7): m/z: [M+H]⁺=495, Rt=0.81 min.

### Example 308

### 6,7-dimethoxy-2-methyl-N-[1-(5-{2-[(4-methyl piperazin-1-yl)methyl]phenyl}thiophen-2-yl)ethyl]quinazolin-4-amine

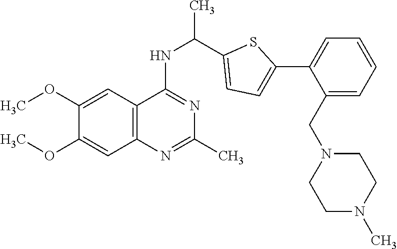 [SMILES: COc1cc2nc(C)nc(NC(C)c3ccc(-c4ccccc4CN4CCN(C)CC4)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), 1-methylpiperazine (26 μL, 230 μmol) and acetic acid (13 μL, 230 μmol) in 1,2-dichloroethane (1.0 mL) was added NaBH(OAc)₃ (48.9 mg, 231 μmol) and the solution stirred at room temperature during 5 hours. The reaction was quenched with aqueous NaOH (1.0 M, 1.0 mL), the mixture extracted with DCM and the solvent removed in vacuo. Purification by preparative HPLC (basic conditions) gave the title compound as a light brown solid (36.2 mg, 61%). 1H-NMR (400 MHz, DMSO-d₆): δ [ppm]=8.16 (d, 1H), 7.65 (s, 1H), 7.38 (dt, 2H), 7.29 (dd, 2H), 7.18 (d, 1H), 7.06 (dd, 1H), 7.05 (s, 1H), 5.97 (quin, 1H), 3.87 (s, 6H), 3.37 (s, 2H), 2.42 (s, 3H), 2.31-2.22 (m, 4H), 2.19-2.07 (m, 4H), 2.03 (s, 3H), 1.71 (d, 3H). LC-MS (method 7): m/z: [M+H]⁺=518, Rt=0.64 min.

### Example 309

### (3S)-{[2-(5-{1-[(6,7-dimethoxy-2-methylquinazolin-4-yl)amino]ethyl}thiophen-2-yl)benzyl]amino}-1-methylpyrrolidin-2-one

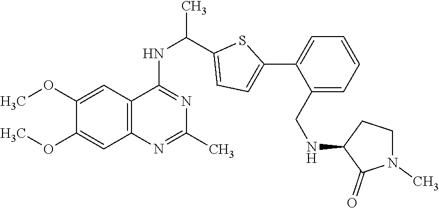

To 2-(5-{1-[(6,7-dimethoxy-2-methylquinazolin-4-yl)amino]ethyl}thiophen-2-yl)benzaldehyde (described in example 179a; 50.0 mg, 115 μmol), (3S)-3-amino-1-methylpyrrolidin-2-one (26.3 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature during 5 hours.

The reaction was quenched with aqueous NaOH (1.0 M, 1.0 mL), the mixture extracted with DCM and the solvent removed in vacuo. Purification by preparative HPLC (basic conditions) gave the title compound as an off-white solid (8.70 mg, 14%). 1H-NMR (400 MHz, DMSO-d₆): δ [ppm]=8.16 (dd, 1H), 7.66-7.64 (m, 1H), 7.63-7.54 (m, 2H), 7.33-7.26 (m, 2H), 7.16 (dd, 1H), 7.08 (dd, 1H), 7.05 (s, 1H), 5.96 (quin, 1H), 3.87 (s, 6H), 3.85-3.75 (m, 2H), 3.23-3.04 (m, 2H), 2.67 (d, 3H), 2.43 (s, 3H), 2.17 (br s, 1H), 2.08-1.97 (m, 1H), 1.71 (d, 3H), 1.56 (dq, 1H). LC-MS (method 7): m/z: [M+H]⁺=532, Rt=0.59 min.

### Example 310

### N-[2-(5-{1-[(6,7-dimethoxy-2-methylquinazolin-4-yl)amino]ethyl}thiophen-2-yl)-5-fluorobenzyl]-1H-pyrazole-3-carboxamide

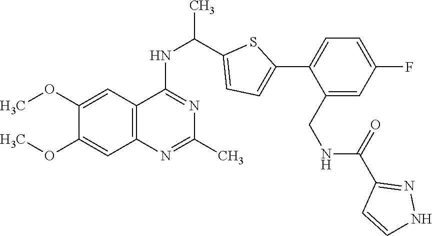 [SMILES: COc1cc2nc(C)nc(NC(C)c3ccc(-c4ccc(F)cc4CNC(=O)c4cc[nH]n4)s3)c2cc1OC]

A solution of N-[1-{5-[2-(aminomethyl)-4-fluorophenyl]thiophen-2-yl}ethyl]-6,7-dimethoxy-2-methylquinazolin-4-amine (described in example 234; 50.0 mg, 110 μmol), 1H-pyrazole-3-carboxylic acid (12.4 mg, 110 μmol), PyBOP (115 mg, 221 μmol) and N,N-diisopropylethylamine (95 μL, 550 μmol) in DMF (1.0 mL) was stirred at room temperature overnight. Purification by preparative HPLC (basic conditions) gave the title compound as an off-white solid (31.6 mg, 51%). ¹H-NMR (400 MHz, DMSO-d₆): δ [ppm]=13.29 (br s, 1H), 8.77 (br s, 1H), 8.17 (d, 1H), 7.87-7.78 (m, 1H), 7.65 (s, 1H), 7.40-7.34 (m, 1H), 7.14-7.07 (m, 4H), 7.05 (s, 1H), 6.70-6.59 (m, 1H), 5.97 (quin, 1H), 4.51 (d, 2H), 3.87 (s, 6H), 2.43 (s, 3H), 1.72 (d, 3H). LC-MS (method 7): m/z: [M+H]⁺=547, Rt=0.83 min.

### Example 311

### 6,7-dimethoxy-2-methyl-N-[1-{5-[2-(morpholin-4-ylmethyl)phenyl]thiophen-2-yl}ethyl]quinazolin-4-amine

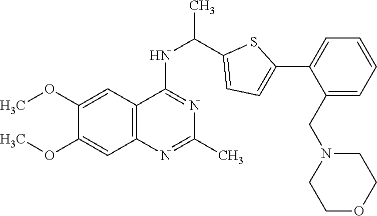 [SMILES: COc1cc2nc(C)nc(NC(C)c3ccc(-c4ccccc4CN4CCOCC4)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), morpholine (20 μL, 230 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) followed by column chromatography (silica gel, MeOH/EtOAc 0-20% then MeOH) gave the title compound as a white solid (24.9 mg, 43%). 1H-NMR (400 MHz, DMSO-d₆): δ [ppm]=8.16 (d, 1H), 7.65 (s, 1H), 7.43-7.36 (m, 2H), 7.32-7.27 (m, 2H), 7.19 (d, 1H), 7.07 (dd, 1H), 7.05 (s, 1H), 5.97 (quin, 1H), 3.87 (s, 3H), 3.87 (s, 3H), 3.43 (br t, 4H), 3.41 (s, 2H), 2.42 (s, 3H), 2.30 (br s, 4H), 1.72 (d, 3H). LC-MS (method 7): m/z: [M+H]⁺=505, Rt=0.60 min.

### Example 312

### 1-[2-(5-{1-[(6,7-dimethoxy-2-methylquinazolin-4-yl)amino]ethyl}thiophen-2-yl)benzyl]azetidin-3-ol

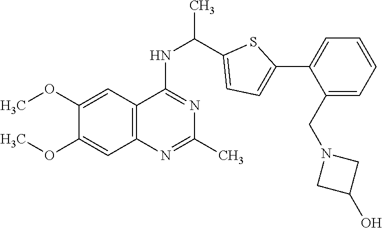 [SMILES: COc1cc2nc(C)nc(NC(C)c3ccc(-c4ccccc4CN4CC(O)C4)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), azetidin-3-ol hydrochloride (1:1) (25.3 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by column chromatography (silica gel, MeOH/EtOAc 0-20% then MeOH) gave the title compound as a light orange solid (28.3 mg, 48%). ¹H-NMR (400 MHz, DMSO-d₆): δ [ppm]=8.19-8.11 (m, 1H), 7.65 (s, 1H), 7.40 (dd, 1H), 7.35-7.32 (m, 1H), 7.31-7.23 (m, 2H), 7.16 (d, 1H), 7.08 (dd, 1H), 7.05 (s, 1H), 5.96 (quin, 1H), 5.27 (d, 1H), 4.15 (sxt, 1H), 3.87 (s, 6H), 3.58 (s, 2H), 3.47 (dt, 2H), 2.76-2.70 (m, 2H), 2.44 (s, 3H), 1.72 (d, 3H). LC-MS (method 7): m/z: [M+H]⁺=491, Rt=0.56 min.

### Example 313

### 2-[2-(5-{1-[(6,7-dimethoxy-2-methylquinazolin-4-yl)amino]ethyl}thiophen-2-yl)benzyl]-2,5,7-triazaspiro[3.4]octan-6-one

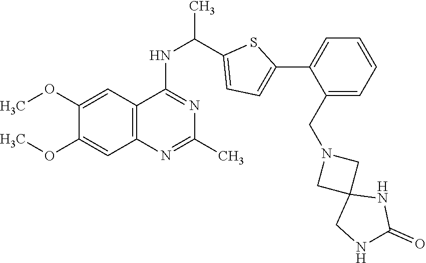 [SMILES: COc1cc2nc(C)nc(NC(C)c3ccc(-c4ccccc4CN4CC5(CNC(=O)N5)C4)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), 2,5,7-triazaspiro[3.4]octan-6-one (29.3 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a light orange solid (19.3 mg, 29%). 1H-NMR (400 MHz, DMSO-d₆): δ [ppm]=8.15 (d, 1H), 7.65 (s, 1H), 7.41 (dd, 1H), 7.35-7.24 (m, 3H), 7.14 (d, 1H), 7.08 (dd, 1H), 7.05 (s, 1H), 6.82 (s, 1H), 6.24 (s, 1H), 5.97 (quin, 1H), 3.87 (s, 6H), 3.58 (s, 2H), 3.46 (s, 2H), 3.31-3.28 (m, 2H), 2.99 (dd, 2H), 2.43 (s, 3H), 1.75-1.70 (m, 3H). LC-MS (method 7): m/z: [M+H]⁺=545, Rt=0.54 min.

### Example 314

### N-[2-(5-{1-[(6,7-dimethoxy-2-methylquinazolin-4-yl)amino]ethyl}thiophen-2-yl)-5-fluorobenzyl]-L-prolinamide

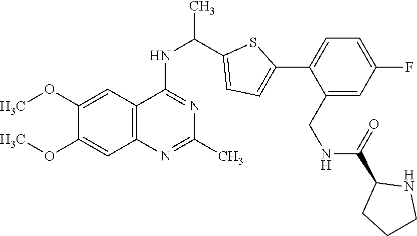 [SMILES: COc1cc2nc(C)nc(NC(C)c3ccc(-c4ccc(F)cc4CNC(=O)[C@@H]4CCCN4)s3)c2cc1OC]

A solution of N-[1-{5-[2-(aminomethyl)-4-fluorophenyl]thiophen-2-yl}ethyl]-6,7-dimethoxy-2-methylquinazolin-4-amine (described in example 234; 50.0 mg, 110 μmol), L-proline (12.7 mg, 110 μmol), PyBOP (115 mg, 221 μmol) and N,N-diisopropylethylamine (95 μL, 550 μmol) in DMF (1.0 mL) was stirred at room temperature overnight. Purification by preparative HPLC (basic conditions) followed by column chromatography (silica gel, MeOH/EtOAc 0-20% then MeOH) gave the title compound as a white solid (9.40 mg, 15%). 1H-NMR (400 MHz, DMSO-d₆): δ [ppm]=8.42 (br td, 1H), 8.15 (d, 1H), 7.64 (s, 1H), 7.35 (dd, 1H), 7.14-7.00 (m, 5H), 5.95 (quin, 1H), 4.33 (d, 2H), 3.87 (s, 6H), 3.54 (dd, 1H), 2.85-2.70 (m, 2H), 2.43 (s, 3H), 1.97-1.87 (m, 1H), 1.71 (d, 3H), 1.67-1.51 (m, 3H). LC-MS (method 7): m/z: [M+H]⁺=550, Rt=0.64 min.

### Example 315

### N-{1-[5-(2-{[(2,2-difluoroethyl)amino]methyl}phenyl)thiophen-2-yl]ethyl}-6,7-dimethoxy-2-methylquinazolin-4-amine

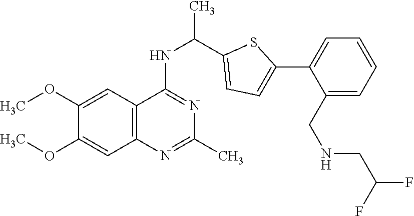 [SMILES: COc1cc2nc(C)nc(NC(C)c3ccc(-c4ccccc4CNCC(F)F)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), 2,2-difluoroethanamine (16 μL, 230 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) followed by column chromatography (silica gel, MeOH/EtOAc 0-20% then MeOH) gave the title compound as a white solid (12.0 mg, 20%). 1H-NMR (400 MHz, DMSO-d₆): δ [ppm]=8.15 (d, 1H), 7.65 (s, 1H), 7.54-7.50 (m, 1H), 7.35-7.30 (m, 2H), 7.29-7.24 (m, 1H), 7.15 (d, 1H), 7.08 (dd, 1H), 7.05 (s, 1H), 5.97 (tt, 1H), 5.96 (quin, 1H), 3.87 (s, 6H), 3.78 (br d, 2H), 2.84 (br t, 2H), 2.43 (s, 3H), 1.71 (d, 3H). LC-MS (method 7): m/z: [M+H]⁺=499, Rt=0.70 min.

### Example 316

### N-[2-(5-{1-[(6,7-dimethoxy-2-methylquinazolin-4-yl)amino]ethyl}thiophen-2-yl)-5-fluorobenzyl]-prolinamide

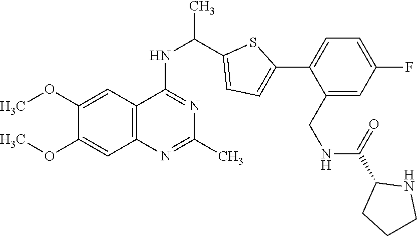 [SMILES: COc1cc2nc(C)nc(NC(C)c3ccc(-c4ccc(F)cc4CNC(=O)[C@H]4CCCN4)s3)c2cc1OC]

A solution of N-[1-{5-[2-(aminomethyl)-4-fluorophenyl]thiophen-2-yl}ethyl]-6,7-dimethoxy-2-methylquinazolin-4-amine (described in example 234; 50.0 mg, 110 μmol), D-proline (12.7 mg, 110 μmol), PyBOP (115 mg, 221 μmol) and N,N-diisopropylethylamine (95 μL, 550 μmol) in DMF (1.0 mL) was stirred at room temperature overnight. Purification by preparative HPLC (basic conditions) followed by column chromatography (silica gel, MeOH/EtOAc 0-20% then MeOH) gave the title compound as a white solid (7.30 mg, 12%). 1H-NMR (400 MHz, DMSO-d₆): δ [ppm]=8.45-8.39 (m, 1H), 8.15 (d, 1H), 7.64 (s, 1H), 7.35 (dd, 1H), 7.14-7.00 (m, 5H), 5.95 (quin, 1H), 4.33 (d, 2H), 3.87 (s, 6H), 3.54 (dd, 1H), 2.85-2.71 (m, 2H), 2.43 (s, 3H), 1.98-1.86 (m, 1H), 1.71 (d, 3H), 1.68-1.50 (m, 3H). LC-MS (method 7): m/z: [M+H]⁺=550, Rt=0.64 min.

### Example 317

### N-[1-{5-[2-(azetidin-1-ylmethyl)phenyl]thiophen-2-yl}ethyl]-6,7-dimethoxy-2-methylquinazolin-4-amine

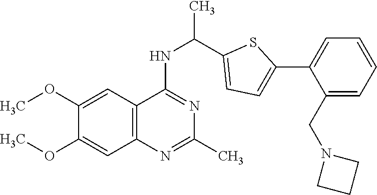 [SMILES: COc1cc2nc(C)nc(NC(C)c3ccc(-c4ccccc4CN4CCC4)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), azetidine (13.2 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a white solid (26.1 mg, 48%). 1H-NMR (400 MHz, DMSO-d₆): δ [ppm]=8.15 (d, 1H), 7.65 (s, 1H), 7.40 (dd, 1H), 7.35-7.32 (m, 1H), 7.31-7.23 (m, 2H), 7.17 (d, 1H), 7.08 (dd, 1H), 7.05 (s, 1H), 5.96 (quin, 1H), 3.87 (s, 6H), 3.53 (s, 2H), 3.09 (t, 4H), 2.44 (s, 3H), 1.93 (quin, 2H), 1.72 (d, 3H). LC-MS (method 7): m/z: [M+H]⁺=475, Rt=0.57 min.

### Example 318

### {1-[(2S)-(5-{1-[(6,7-dimethoxy-2-methylquinazolin-4-yl)amino]ethyl}thiophen-2-yl)benzyl]azetidin-2-yl}methanol

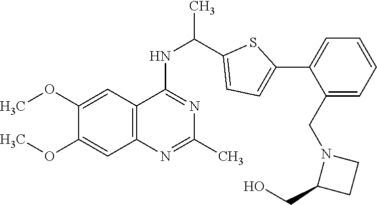 [SMILES: COc1cc2nc(C)nc(NC(C)c3ccc(-c4ccccc4CN4CC[C@H]4CO)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), (2S)-azetidin-2-ylmethanol (20.1 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a white solid (32.3 mg, 55%). 1H-NMR (400 MHz, DMSO-d₆): δ [ppm]=8.15 (d, 1H), 7.65 (d, 1H), 7.48-7.44 (m, 1H), 7.34-7.29 (m, 1H), 7.29-7.22 (m, 3H), 7.07 (ddd, 1H), 7.05 (s, 1H), 5.96 (quin, 1H), 4.35 (td, 1H), 3.88-3.86 (m, 6H), 3.82 (d, 1H), 3.47 (dd, 1H), 3.30-3.26 (m, 2H), 3.25-3.18 (m, 1H), 3.17-3.10 (m, 1H), 2.75-2.68 (m, 1H), 2.43 (d, 3H), 1.96-1.87 (m, 1H), 1.80 (quin, 1H), 1.72 (d, 3H). LC-MS (method 7): m/z: [M+H]⁺=505, Rt=0.56 min.

### Example 319

### N-{1-[5-(2-{[3-(dimethylamino)azetidin-1-yl]methyl}phenyl)thiophen-2-yl]ethyl}-6,7-dimethoxy-2-methylquinazolin-4-amine

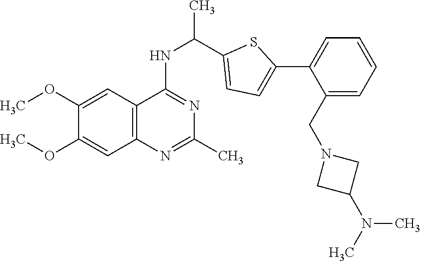

To 2-(5-{1-[(6,7-dimethoxy-2-methylquinazolin-4-yl)amino]ethyl}thiophen-2-yl)benzaldehyde (described in example 179a; 50.0 mg, 115 μmol), N,N-dimethylazetidin-3-amine dihydrochloride (39.9 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a white solid (22.3 mg, 36%). 1H-NMR (400 MHz, DMSO-d₆): δ [ppm]=8.15 (d, 1H), 7.65 (s, 1H), 7.41-7.37 (m, 1H), 7.37-7.33 (m, 1H), 7.31-7.24 (m, 2H), 7.15 (d, 1H), 7.08 (dd, 1H), 7.05 (s, 1H), 5.97 (quin, 1H), 3.88-3.87 (m, 3H), 3.87-3.85 (m, 3H), 3.55 (s, 2H), 3.30-3.25 (m, 2H), 2.77-2.70 (m, 2H), 2.70-2.67 (m, 1H), 2.43 (s, 3H), 1.95 (s, 6H), 1.72 (d, 3H). LC-MS (method 7): m/z: [M+H]⁺=518, Rt=0.57 min.

### Example 320

### N-[1-(5-{2-[(3,3-difluoroazetidin-1-yl)methyl]phenyl}thiophen-2-yl)ethyl]-6,7-dimethoxy-2-methylquinazolin-4-amine

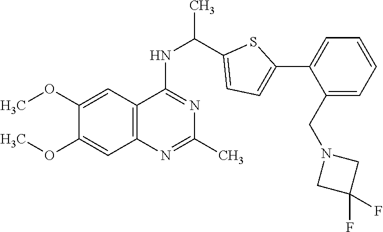 [SMILES: COc1cc2nc(C)nc(NC(C)c3ccc(-c4ccccc4CN4CC(F)(F)C4)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), 3,3-difluoroazetidine hydrochloride (1:1) (29.9 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a white solid (29.1 mg, 49%). 1H-NMR (400 MHz, DMSO-d₆): δ [ppm]=8.15 (d, 1H), 7.65 (s, 1H), 7.48-7.44 (m, 1H), 7.38-7.35 (m, 1H), 7.35-7.27 (m, 2H), 7.16 (d, 1H), 7.10 (dd, 1H), 7.05 (s, 1H), 5.96 (quin, 1H), 3.87 (s, 6H), 3.75 (s, 2H), 3.59 (t, 4H), 2.43 (s, 3H), 1.72 (d, 3H). LC-MS (method 7): m/z: [M+H]⁺=511, Rt=0.90 min.

### Example 321

### 6,7-di methoxy-2-methyl-N-{1-[5-(2-{[methyl(2,2,2-trifluoroethyl)amino]methyl}phenyl)thiophen-2-yl]ethyl}quinazolin-4-amine

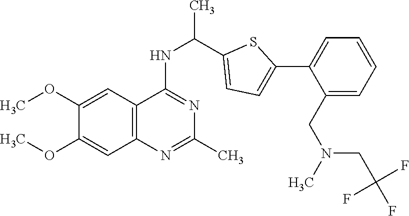 [SMILES: COc1cc2nc(C)nc(NC(C)c3ccc(-c4ccccc4CN(C)CC(F)(F)F)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), 2,2,2-trifluoro-N-methylethanamine hydrochloride (1:1) (34.5 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a white solid (25.4 mg, 42%). 1H-NMR (400 MHz, DMSO-d₆): δ [ppm]=8.14 (d, 1H), 7.65 (s, 1H), 7.54-7.49 (m, 1H), 7.38-7.27 (m, 3H), 7.08 (s, 2H), 7.05 (s, 1H), 5.96 (quin, 1H), 3.87 (s, 6H), 3.74 (s, 2H), 3.20 (q, 2H), 2.43 (s, 3H), 2.25 (s, 3H), 1.72 (d, 3H). LC-MS (method 7): m/z: [M+H]⁺=531, Rt=1.09 min.

### Example 322

### N-[1-(5-{2-[(3-fluoroazetidin-1-yl)methyl]phenyl}thiophen-2-yl)ethyl]-6,7-dimethoxy-2-methylquinazolin-4-amine

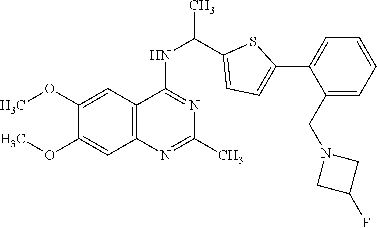 [SMILES: COc1cc2nc(C)nc(NC(C)c3ccc(-c4ccccc4CN4CC(F)C4)s3)c2cc1OC]

To 2-(5-{1-[(6,7-dimethoxy-2-methylquinazolin-4-yl)amino]ethyl}thiophen-2-yl)benzaldehyde (described in example 179a; 50.0 mg, 115 μmol), 3-fluoroazetidine hydrochloride (1:1) (25.7 mg, 231 μmol) and acetic acid (13 μL, 230 μmol) in 1,2-dichloroethane (1.0 mL) was added NaBH(OAc)₃ (48.9 mg, 231 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a white solid (28.6 mg, 50%). 1H-NMR (400 MHz, DMSO-d₆): δ [ppm]=8.15 (d, 1H), 7.65 (s, 1H), 7.42 (dd, 1H), 7.37-7.33 (m, 1H), 7.33-7.25 (m, 2H), 7.16 (d, 1H), 7.09 (dd, 1H), 7.05 (s, 1H), 5.96 (quin, 1H), 5.13 (dquin, 1H), 3.87 (s, 6H), 3.65 (s, 2H), 3.57 3.47 (m, 2H), 3.18-3.13 (m, 1H), 3.12-3.06 (m, 1H), 2.43 (s, 3H), 1.72 (d, 3H). LC-MS (method 7): m/z: [M+H]⁺=493, Rt=0.58 min.

### Example 323

### N-[1-(5-{4-chloro-2-[(dimethylamino)methyl]phenyl}thiophen-2-yl)ethyl]-6,7-dimethoxy-2-methylquinazolin-4-amine

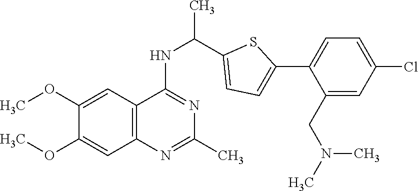 [SMILES: COc1cc2nc(C)nc(NC(C)c3ccc(-c4ccc(Cl)cc4CN(C)C)s3)c2cc1OC]

Under argon, N-[1-(5-bromothiophen-2-yl)ethyl]-6,7-dimethoxy-2-methylquinazolin-4-amine hydrochloride (1:1) (described in example 209; 100 mg, 225 μmol), 1-[5-chloro-2-(4,4,5,5-tetramethyl-1,3,2-dioxaborolan-2-yl)phenyl]-N,N-dimethylmethanamine (66.5 mg, 225 μmol), K₂SO₃ (124 mg, 899 μmol) and Pd(PPh₃)₄ (26.0 mg, 22.5 μmol) in dioxane (2.3 mL) and H₂O (460 μL) were stirred at 110° C. overnight. H₂O was added, the mixture extracted with DCM and the solvent removed in vacuo. Purification by column chromatography (silica gel, MeOH/EtOAc 0-20% then MeOH, repeated twice) followed by preparative HPLC (basic conditions) gave the title compound as a yellow solid (37.5 mg, 33%). 1H-NMR (400 MHz, DMSO-d₆): δ [ppm]=8.15 (d, 1H), 7.64 (s, 1H), 7.50 (d, 1H), 7.37 (d, 1H), 7.34 (dd, 1H), 7.15 (d, 1H), 7.09 (dd, 1H), 7.05 (s, 1H), 5.95 (quin, 1H), 3.87 (s, 3H), 3.87 (s, 3H), 3.38 (s, 2H), 2.43 (s, 3H), 2.11 (s, 6H), 1.71 (d, 3H). LC-MS (method 7): m/z: [M+H]⁺=497, Rt=0.60 min.

### Example 324

### 1-[2-(5-{1-[(6,7-dimethoxy-2-methylquinazolin-4-yl)amino]ethyl}thiophen-2-yl)phenyl]ethanone

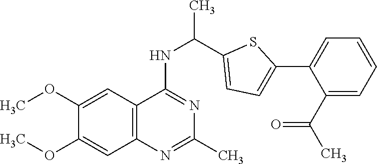 [SMILES: COc1cc2nc(C)nc(NC(C)c3ccc(-c4ccccc4C(C)=O)s3)c2cc1OC]

Under argon, N-[1-(5-bromothiophen-2-yl)ethyl]-6,7-dimethoxy-2-methylquinazolin-4-amine (described in example 209; 200 mg, 85% purity, 416 μmol), (2-acetylphenyl)boronic acid (68.3 mg, 416 μmol), K₂CO₃ (230 mg, 1.67 mmol) and Pd(PPh₃)₄ (48.1 mg, 41.6 μmol) in dioxane (4.0 mL) and H₂O (800 μL) were stirred at 110° C. overnight. H₂O was added, the mixture extracted with DCM and the solvent removed in vacuo. Purification by preparative HPLC (basic conditions) gave the title compound as a light yellow solid (71.3 mg, 38%). ¹H-NMR (400 MHz, DMSO-d₆): δ [ppm]=8.15 (d, 1H), 7.64 (s, 1H), 7.53-7.40 (m, 4H), 7.07 (dd, 1H), 7.05 (s, 1H), 6.89 (d, 1H), 5.93 (quin, 1H), 3.87 (s, 6H), 2.43 (s, 3H), 2.21 (s, 3H), 1.71 (d, 3H). LC-MS (method 7): m/z: [M+H]⁺=448, Rt=0.92 min.

### Example 325

### 6,7-di methoxy-2-methyl-N-[1-(5-{2-[2-(pyrrolidin-1-yl)ethoxy]phenyl}thiophen-2-yl)ethyl]quinazolin-4-amine

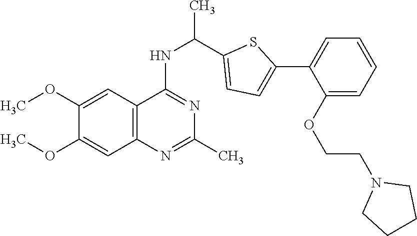 [SMILES: COc1cc2nc(C)nc(NC(C)c3ccc(-c4ccccc4OCCN4CCCC4)s3)c2cc1OC]

Under argon, N-[1-(5-bromothiophen-2-yl)ethyl]-6,7-dimethoxy-2-methylquinazolin-4-amine hydrochloride (1:1) (described in example 209; 100 mg, 225 μmol), {2-[2-(pyrrolidin-1-yl)ethoxy]phenyl}boronic acid (52.9 mg, 225 μmol), K₂CO₃ (124 mg, 899 μmol) and Pd(PPh₃)₄ (26.0 mg, 22.5 μmol) in dioxane (2.3 mL) and H₂O (460 μL) were stirred at 110° C. overnight. H₂O was added, the mixture extracted with DCM and the solvent removed in vacuo. Purification by preparative HPLC (basic conditions) gave the title compound as a off-white solid (81.1 mg, 70%). ¹H-NMR (400 MHz, DMSO-d₆): δ [ppm]=8.12 (d, 1H), 7.67-7.63 (m, 2H), 7.47 (d, 1H), 7.24-7.19 (m, 1H), 7.08 (dd, 1H), 7.05 (s, 1H), 7.03 (dd, 1H), 6.96 (td, 1H), 5.96 (quin, 1H), 4.10 (t, 2H), 3.87 (s, 6H), 2.77-2.69 (m, 2H), 2.43 (s, 3H), 2.35-2.31 (m, 4H), 1.70 (d, 3H), 1.51 (ddd, 4H). LC-MS (method 7): m/z: [M+H]⁺=519, Rt=0.62 min.

### Example 326

### 6,7-dimethoxy-2-methyl-N-[1-(4-{2-[(methylamino)methyl]phenyl}thiophen-2-yl)ethyl]quinazolin-4-amine, Enantiomer 1

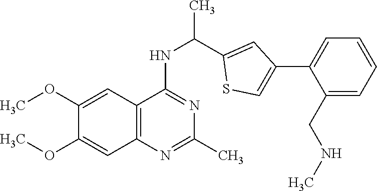 [SMILES: CNCc1ccccc1-c1csc(C(C)Nc2nc(C)nc3cc(OC)c(OC)cc23)c1]

Enantiopure 6,7-Dimethoxy-2-methyl-N-[1-(4-{2-[(methylamino)methyl]phenyl}thiophen-2-yl)ethyl]quinazolin-4-amine was obtained from racemic 6,7-dimethoxy-2-methyl-N-[1-(4-{2-[(methylamino)methyl]phenyl}thiophen-2-yl)ethyl]quinazolin-4-amine (described in example 271) by chiral HPLC purification (method X1). ¹H-NMR (400 MHz, DMSO-d₆): δ [ppm]=8.14 (d, 1H), 7.65 (s, 1H), 7.46 (d, 1H), 7.46-7.43 (m, 1H), 7.32-7.24 (m, 4H), 7.05 (s, 1H), 5.97 (quin, 1H), 3.87 (s, 3H), 3.87 (s, 3H), 3.57 (s, 2H), 2.43 (s, 3H), 2.22 (s, 3H), 1.72 (d, 3H). LC-MS (method 7): m/z: [M+H]⁺=449, Rt=0.55 min. [α]_{D}=−93.9°+/−1.16°.

### Example 327

### 6,7-dimethoxy-2-methyl-N-[1-(4-{2-[(methylamino)methyl]phenyl}thiophen-2-yl)ethyl]quinazolin-4-amine, Enantiomer 2

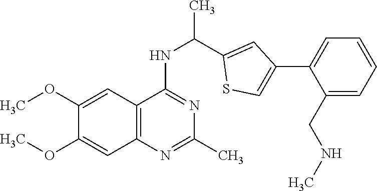 [SMILES: CNCc1ccccc1-c1csc(C(C)Nc2nc(C)nc3cc(OC)c(OC)cc23)c1]

Enantiopure 6,7-Dimethoxy-2-methyl-N-[(1-(4-{2-[(methylamino)methyl]phenyl}thiophen-2-yl)ethyl]quinazolin-4-amine was obtained from racemic 6,7-dimethoxy-2-methyl-N-[1-(4-{2-[(methylamino)methyl]phenyl}thiophen-2-yl)ethyl]quinazolin-4-amine (described in example 271) by chiral HPLC purification (method X1). ¹H-NMR (400 MHz, DMSO-d₆): δ [ppm]=8.14 (d, 1H), 7.65 (s, 1H), 7.46 (d, 1H), 7.46-7.42 (m, 1H), 7.32-7.24 (m, 4H), 7.05 (s, 1H), 5.97 (quin, 1H), 3.87 (s, 3H), 3.87 (s, 3H), 3.57 (s, 2H), 2.43 (s, 3H), 2.22 (s, 3H), 1.72 (d, 3H). LC-MS (method 7): m/z: [M+H]⁺=449, Rt=0.55 min. [α]_{D}=+89.4°+/−1.91°.

### Example 328

### N-{1-[5-(6,7-dihydro-5H-pyrrolo[1,2-a]imidazol-3-yl)thiophen-2-yl]ethyl}-6,7-dimethoxy-2-methylquinazolin-4-amine, Enantiomer 1

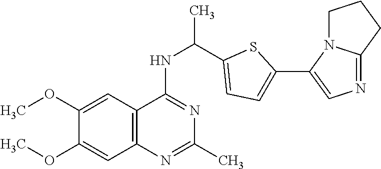 [SMILES: COc1cc2nc(C)nc(NC(C)c3ccc(-c4cnc5n4CCC5)s3)c2cc1OC]

Enantiopure N-{1-[5-(6,7-Dihydro-5H-pyrrolo[1,2-a]imidazol-3-yl)thiophen-2-yl]ethyl}-6,7-dimethoxy-2-methylquinazolin-4-amine was obtained from racemic N-{1-[5-(6,7-Dihydro-5H-pyrrolo[1,2-a]imidazol-3-yl)thiophen-2-yl]ethyl}-6,7-dimethoxy-2-methylquinazolin-4-amine (described in example 219) by chiral HPLC purification (method X2). ¹H-NMR (400 MHz, DMSO-d₆): δ [ppm]=8.11 (d, 1H), 7.63 (s, 1H), 7.05-7.02 (m, 4H), 5.90 (quin, 1H), 4.06 (t, 2H), 3.87 (s, 6H), 2.77-2.71 (m, 2H), 2.59-2.53 (m, 2H), 2.43 (s, 3H), 1.69 (d, 3H). LC-MS (method 9): m/z: [M+H]⁺=436, Rt=0.58 min. [α]_{D}=−92.7°+/−31.06°.

### Example 329

### N-{1-[5-(6,7-dihydro-5H-pyrrolo[1,2-a]imidazol-3-yl)thiophen-2-yl]ethyl}-6,7-dimethoxy-2-methylquinazolin-4-amine, Enantiomer 2

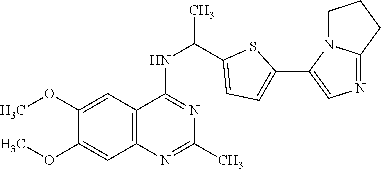 [SMILES: COc1cc2nc(C)nc(NC(C)c3ccc(-c4cnc5n4CCC5)s3)c2cc1OC]

Enantiopure N-{1-[5-(6,7-Dihydro-5H-pyrrolo[1,2-a]imidazol-3-yl)thiophen-2-yl]ethyl}-6,7-dimethoxy-2-methylquinazolin-4-amine was obtained from racemic N-{1-[5-(6,7-Dihydro-5H-pyrrolo[1,2-a]imidazol-3-yl)thiophen-2-yl]ethyl}-6,7-dimethoxy-2-methylquinazolin-4-amine (described in example 219) by chiral HPLC purification (method X2). ¹H-NMR (400 MHz, DMSO-d₆): δ [ppm]=8.11 (d, 1H), 7.63 (s, 1H), 7.06-7.02 (m, 4H), 5.90 (quin, 1H), 4.06 (t, 2H), 3.87 (s, 6H), 2.77-2.71 (m, 2H), 2.59-2.53 (m, 2H), 2.43 (s, 3H), 1.68 (d, 3H). LC-MS (method 9): m/z: [M+H]⁺=436, Rt=0.58 min. [α]_{D}=+154.0°+/−34.5°.

### Example 330

### 6,7-dimethoxy-2-methyl-N-[1-(2-methyl-1-benzothiophen-4-yl)ethyl]quinazolin-4-amine

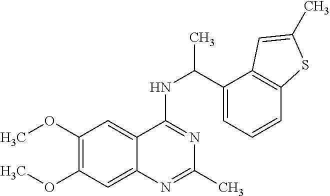

### Step a

### 4-bromo-2-methyl benzo[b]thiophene

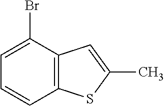

4-Bromobenzo[b]thiophene, 5.00 g (23.5 mmol), was dissolved in 100 mL of tetrahydrofuran. Then lithium diisopropylamide, 14.08 mL (28.2 mmol), was added dropwise at −78° C. The resulting mixture was stirred at this temperature for 30 min. Then iodomethane, 7.30 mL (117.3 mmol), was added dropwise at −78° C. and the resulting mixture was stirred at this temperature for 1 hour. Aq. ammonium chloride was added and the resulting solution was extracted with ethyl acetate. The combined organic phase was dried over anhydrous sodium sulfate and the solvent was removed in vacuo. The residue was purified by silica gel column chromatography to give 4.00 g (71%) of the product as a yellow solid. ¹H-NMR (400 MHz, CDCl₃): δ [ppm]=2.63 (d, 3H), 7.08-7.19 (m, 2H), 7.49 (d, 1H), 7.69 (d, 1H).

### Step b

### 1-(2-methylbenzo[b]thiophen-4-yl)ethanone

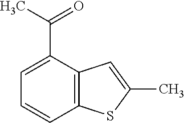

Synthesis of the enolether: 4-Bromo-2-methylbenzo[b]thiophene, 1.50 g (6.6 mmol), tributyl(1-ethoxyvinyl)stannane, 3.12 g (8.6 mmol), 1,1′-bis(diphenylphosphino)ferrocenepalladium(11) chloride, 483 mg (0.7 mmol) and potassium carbonate, 2.74 g (19.8 mmol), were added into 30 mL of 1,4-dioxane. The resulting mixture was stirred at 60° C. for overnight under nitrogen.

Hydrolysis of the enolether: Water was added and the resulting mixture was extracted with ethyl acetate. The combined organic layers were dried over anhydrous sodium sulfate and the solvent was removed in vacuo. The residue was purified by silica gel column chromatography to give 1.00 g (64%) of the product as a yellow solid. MS (ESIpos): m/z=191 [M+H]⁺; LC-MS (method 4, gradient Acetonitrile-Water-0.05% HCOOH-5% B): Rₜ=1.58 min.

### Step c

### (E)-1-(2-methylbenzo[b]thiophen-4-yl)ethanone oxime

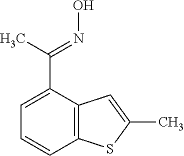

1-(2-Methylbenzo[b]thiophen-4-yl)ethanone, 500 mg (2.6 mmol), hydroxylamine hydrochloride, 274 mg (3.9 mmol), and sodium acetate, 323 mg (3.9 mmol), were dissolved in 10 mL of methanol. The resulting mixture was stirred at room temperature for 2 hours. The solid was removed by filtration and the filtrate was concentrated in vacuo to give 500 mg (81%) of the product as an off-white solid. MS (ESIpos): m/z=206 [M+H]⁺; LC-MS (method 4, gradient Acetonitrile-Water-0.05% HCOOH-5% B): Rₜ=1.49 min.

### Step d

### 1-(2-methylbenzo[b]thiophen-4-yl)ethanamine H₃C NH₂

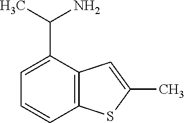

(E)-1-(2-Methylbenzo[b]thiophen-4-yl)ethanone oxime, 500 mg (2.4 mmol), and Raney Nickel, 500 mg (8.5 mmol), were dissolved in 10 mL of 7.0 M ammonium/methanol solution. The resulting mixture was stirred under hydrogen (3 atm) at room temperature for overnight. The solid was removed by filtration and the filtrate was concentrated in vacuo to give 300 mg (53%) of the product as a grey solid. MS (ESIpos): m/z=192 [M+H]⁺; LC-MS (method 4, gradient acetonitrile-water-0.05% TFA-5% B): Rₜ=1.07 min.

### Step e

### 6,7-dimethoxy-2-methyl-N-[1-(2-methyl-1-benzothiophen-4-yl)ethyl]quinazolin-4-amine

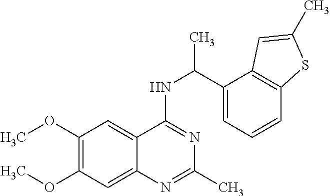

1-(2-Methylbenzo[b]thiophen-4-yl)ethanamine, 100 mg (0.52 mmol), 4-chloro-6,7-dimethoxy-2-methylquinazoline, 137 mg (0.58 mmol, commercially available), and triethylamine, 132 mg (1.31 mmol), were dissolved in 5 mL of N,N-dimethylformamide. The resulting solution was stirred at 100° C. for overnight under nitrogen. Water was added and the resulting mixture was extracted with ethyl acetate. The combined organic phase was dried over anhydrous sodium sulfate and the solvent was removed in vacuo. The residue was purified by preparative HPLC to give 11.60 mg (6%) of the product as a light yellow solid. MS (ESIpos): m/z=394 [M+H]⁺; LC-MS (method 4, gradientAcetonitrile-Water-0.05% TFA-5% B): Rₜ=1.19 min. ¹H-NMR (400 MHz, CD₃OD): δ [ppm]=2.44 (s, 3H), 2.57 (d, 3H), 3.92 (s, 3H), 3.93 (s, 3H), 6.12-6.18 (m, 1H), 7.01 (s, 1H), 7.22 (t, 1H), 7.33 (s, 1H), 7.41-7.43 (m, 1H), 7.61-7.65 (m, 2H).

### Example 331

### 6,7-dimethoxy-2-methyl-N-[1-(thieno[2,3-b]pyridin-4-yl)ethyl]quinazolin-4-amine

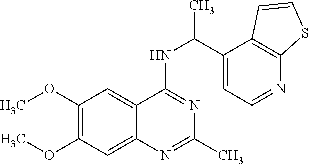 [SMILES: COc1cc2nc(C)nc(NC(C)c3ccnc4sccc34)c2cc1OC]

### Step a

### 4-(1-ethoxyvinyl)thieno[2,3-b]pyridine

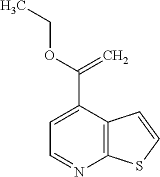

This compound was synthesized by the same method as described in example 330 (step b; synthesis of enolether) to give 400 mg (63%) of the title compound as a yellow solid. MS (ESIpos): m/z=206 [M+H]⁺; LC-MS (method 4, gradient Acetonitrile-Water-0.05% HCOOH-5% B): Rₜ=1.86 min.

### Step b

### 1-(thieno[2,3-b]pyridin-4-yl)ethanone

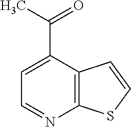

4-(1-Ethoxyvinyl)thieno[2,3-b]pyridine, 400 mg (2.0 mmol), was dissolved in 10 mL of tetrahydrofuran. Then 5 mL of hydrochloric acid (4.0 M) was added. The resulting mixture was stirred at 50° C. for 2 hours. The mixture was extracted with ethyl acetate and the combined organic phase was dried over anhydrous sodium sulfate. The solvent was removed in vacuo and the residue was purified by silica gel column chromatography to give 250 mg (72%) of the product as a yellow solid. MS (ESIpos): m/z=178 [M+H]⁺; LC-MS (method 4, gradient Acetonitrile-Water-0.1% HCOOH-10% B): Rₜ=0.79 min.

### Step c

### (E)-1-(thieno[2,3-b]pyridin-4-yl)ethanone oxime

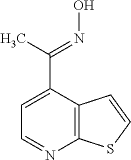

This compound was synthesized by the same method as described in example 330 (step c) to give 300 mg (75%) of the product as a yellow oil. MS (ESIpos): m/z=193 [M+H]⁺; LC-MS (method 4, Acetonitrile-Water-0.05% TFA-5% B): Rₜ=1.08 min.

### Step d

### 1-(Thieno[2,3-b]pyridin-4-yl)ethanamine

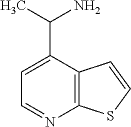

This compound was synthesized by the same method as described in example 330 (step d) to give 200 mg (48%) of the product as a yellow solid. MS (ESIpos): m/z=179 [M+H]⁺; LC-MS (method 4, gradient starting with Acetonitrile-Water-0.05% TFA-5% B): Rₜ=0.66 min.

### Step e

### 6,7-dimethoxy-2-methyl-N-[1-(thieno[2,3-b]pyridin-4-yl)ethyl]quinazolin-4-amine

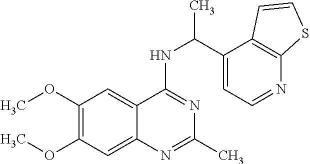 [SMILES: COc1cc2nc(C)nc(NC(C)c3ccnc4sccc34)c2cc1OC]

This compound was synthesized by the same method as described in example 330 (step e) to give 21.2 mg (10%) of the product as a light yellow solid. MS (ESIpos): m/z=381 [M+H]⁺; LC-MS (method 4, gradient starting with Acetonitrile-Water-0.05% TFA-5% B): Rₜ=0.90 min. ¹H-NMR (400 MHz, CD₃OD): δ [ppm]=1.77 (d, 3H), 2.33 (s, 3H), 3.94 (s, 3H), 4.01 (s, 3H), 6.09 6.14 (m, 1H), 7.02 (s, 1H), 7.47 (d, 1H), 7.67 (s, 1H), 7.72-7.76 (m, 2H), 8.45 (d, 1H).

### Example 332

### 6,7-dimethoxy-2-methyl-N-[1-(thieno[2,3-c]pyridin-4-yl)ethyl]quinazolin-4-amine

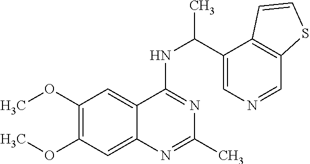 [SMILES: COc1cc2nc(C)nc(NC(C)c3cncc4sccc34)c2cc1OC]

### Step a

### ethyl 4-bromothieno[2,3-c]pyridine-2-carboxylate

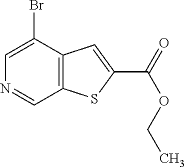

3,5-Dibromoisonicotinaldehyde, 2.00 g (7.6 mmol), ethyl 2-mercaptoacetate, 0.91 g (7.6 mmol), and cesium carbonate, 2.46 g (7.6 mmol), were added into 40 mL of tetrahydrofuran. The resulting mixture was stirred at 60° C. for overnight. After evaporation in vacuo, water was added and the mixture was extracted with ethyl acetate. The combined organic phase was dried over anhydrous sodium sulfate and the solvent was removed in vacuo to give 1.8 g (82%) of the product as a yellow solid. MS (ESIpos): m/z=286 [M+H]⁺; LC-MS (method 4, gradient starting with Acetonitrile-Water-0.05% HCOOH-5% B): Rₜ=1.47 min.

### Step b

### 4-bromothieno[2,3-c]pyridine-2-carboxylic acid

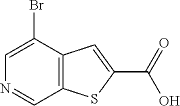 [SMILES: O=C(O)c1cc2c(Br)cncc2s1]

Ethyl 4-bromothieno[2,3-c]pyridine-2-carboxylate, 1.80 g (6.3 mmol), and sodium hydroxide, 1.13 g (28.3 mmol), were dissolved in 50 mL of methanol and 25 mL of water. The resulting mixture was stirred at room temperature for overnight. After evaporation in vacuo, hydrochloric acid (4.0 M) was added to adjust the pH=2 and the precipitated solid was collected by filtration. The filter cake was washed with water and dried in vacuo to give 1.50 g (74%) of the product as a yellow solid. MS (ESIpos): m/z=258 [M+H]⁺; LC-MS (method 4, gradient starting with Acetonitrile-Water-0.05% HCOOH-5% B): Rₜ=1.17 min.

### Step c

### 4-bromothieno[2,3-c]pyridine

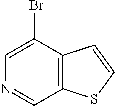

4-Bromothieno[2,3-c]pyridine-2-carboxylic acid, 1.30 g (5.0 mmol), silver carbonate, 139 mg (0.5 mmol), and acetic acid, 0.04 mL (0.6 mmol), were dissolved in 13 mL of N,N-dimethylformamide. The resulting mixture was stirred at 120° C. for overnight. Water was added and the resulting mixture was extracted with ethyl acetate. The combined organic phase was dried over anhydrous sodium sulfate and the solvent was removed in vacuo. The residue was purified by silica gel column chromatography to give 400 mg (35%) of the product as a yellow solid. MS (ESIpos): m/z=214 [M+H]⁺; LC-MS (Acetonitrile-Water-0.05% HCOOH-5% B): Rₜ=1.16 min.

### Step d

### 4-(1-ethoxyvinyl)thieno[2,3-c]pyridine

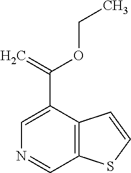

This compound was synthesized by the same method as described in example 330 (step b, synthesis of enolether) to give 380 mg (73%) of the product as a yellow oil. MS (ESIpos): m/z=206 [M+H]⁺; LC-MS (method 4, gradient starting with Acetonitrile-Water-0.05% TFA-5% B): Rₜ=0.82 min.

### Step e

### 1-(thieno[2,3-c]pyridin-4-yl)ethanone

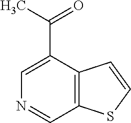

This compound was synthesized by the same method as described in example 330 (step b) to give 200 mg (59%) of the product as yellow oil. MS (ESIpos): m/z=178 [M+H]⁺; LC-MS (method 4, gradient starting with Acetonitrile-Water-0.05% TFA-5% B): Rₜ=0.44 min.

### Step f

### 1-(thieno[2,3-c]pyridin-4-yl)ethanamine

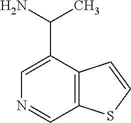

1-(Thieno[2,3-c]pyridin-4-yl)ethanone, 200 mg (1.9 mmol), and 1.2 mL of acetic acid were dissolved in 10 mL of 7 M ammonium/methanol solution. Then sodium cyanoborohydride, 284 mg (4.5 mmol), was added and the resulting mixture was stirred at 40° C. for overnight. The solvent was removed in vacuo and the residue was purified by silica gel column chromatography to give 150 mg (59%, 82% purity) of the product as a yellow oil and the product was used directly for next step. MS (ESIpos): m/z=179 [M+H]⁺; LC-MS (method 4, gradient starting with Acetonitrile-Water-0.05% TFA-5% B): Rₜ=0.61 min.

### Step g

### 6,7-dimethoxy-2-methyl-N-[1-(thieno[2,3-c]pyridin-4-yl)ethyl]quinazolin-4-amine

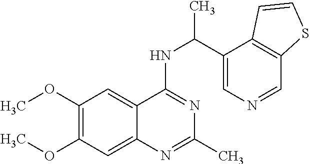 [SMILES: COc1cc2nc(C)nc(NC(C)c3cncc4sccc34)c2cc1OC]

1-(Thieno[2,3-c]pyridin-4-yl)ethanamine, 50 mg (0.28 mmol), 4-chloro-6,7-dimethoxy-2-methylquinazoline, 74 mg (commercially available; 0.31 mmol), triethylamine, 85 mg (0.84 mmol), were dissolved in 3 mL of methanol. The resulting mixture was stirred at room temperature for 3 days under nitrogen and the solid was removed by filtration. The filtrate was purified by preparative HPLC to give 13.80 mg (13%) of the product as a light yellow solid. MS (ESIpos): m/z=381 [M+H]⁺; LC-MS (method 4, gradient starting with Acetonitrile-Water-0.05% TFA-5% B): Rₜ=0.66 min. ¹H-NMR (400 MHz, CD₃OD): δ [ppm]=1.84 (d, 3H), 2.43 (s, 3H), 3.95 (s, 3H), 3.96 (s, 3H), 6.20-6.25 (m, 1H), 7.02 (s, 1H), 7.66 (s, 1H), 7.83 (d, 1H), 8.04 (d, 1H), 8.51 (s, 1H), 9.06 (s, 1H).

### Example 333

### 6,7-dimethoxy-2-methyl-N-[1-(thieno[3,2-c]pyridin-4-yl)ethyl]quinazolin-4-amine

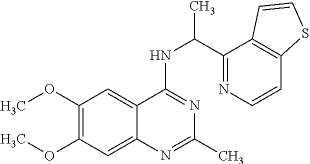 [SMILES: COc1cc2nc(C)nc(NC(C)c3nccc4sccc34)c2cc1OC]

### Step a Br

### 4-bromothieno[3,2-c]pyridine

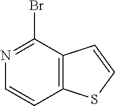

Thieno[3,2-c]pyridin-4(5H)-one, 700.00 mg (4.6 mmol), was dissolved in 30 mL of toluene.

Then phosphoryl bromide, 1.59 g (5.6 mmol), and 0.1 mL of N,N-dimethylformamide were added successively. The resulting mixture was stirred at 110° C. for 2 hours. The reaction was quenched with water and the resulting solution was extracted with ethyl acetate. The combined organic phase was dried over anhydrous sodium sulfate. The solvent was removed in vacuo and the residue was purified by silica gel column chromatography to give 600 mg (56%) of the product as a light yellow solid. MS (ESIpos): m/z=214 [M+H]⁺; LC-MS (method 4, gradient starting with Acetonitrile-Water-0.05% HCOOH-5% B): Rₜ=0.83 min.

### Step b

### 4-(1-ethoxyvinyl)thieno[3,2-c]pyridine

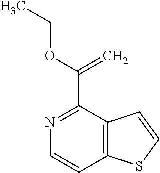

This compound was synthesized by the same method as described in example 330 (step b, synthesis of enolether) to give 430 mg (70%) of the product as yellow oil. MS (ESIpos): m/z=206 [M+H]⁺; LC-MS (method 4, gradient starting with Acetonitrile-Water-0.1% HCOOH-10% B): Rₜ=0.57 min.

### Step c

### 1-(thieno[3,2-c]pyridin-4-yl)ethanone

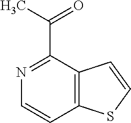

This compound was synthesized by the same method as described in example 330 (step b, hydrolysis of enolether) to give 350.00 mg (82%) of the product as a yellow solid. MS (ESIpos): m/z=178 [M+H]⁺; LC-MS (method 4, gradient starting with Acetonitrile-Water-0.05% TFA-5% B): Rₜ=0.79 min.

### Step d

### 1-(thieno[3,2-c]pyridin-4-yl)ethanamine

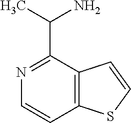

This compound was synthesized by the same method as described in example 332 (step f) to give 200.00 mg (55%) of the product as a yellow solid. MS (ESIpos): m/z=179 [M+H]⁺; LC-MS (method 4, gradient starting with Acetonitrile-Water-0.05% TFA-5% B): Rₜ=0.52 min.

### Step e

### 6,7-dimethoxy-2-methyl-N-[1-(thieno[3,2-c]pyridin-4-yl)ethyl]quinazolin-4-amine

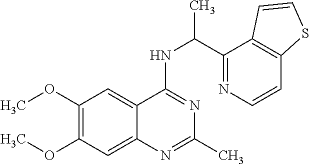 [SMILES: COc1cc2nc(C)nc(NC(C)c3nccc4sccc34)c2cc1OC]

This compound was synthesized by the same method as described in example 332 (step g) to give 12.70 mg (6%) of the product as a white solid. MS (ESIpos): m/z=381[M+H]⁺; LC-MS (method 4, gradient starting with Acetonitrile-Water-0.05% TFA-5% B): Rₜ=0.82 min. ¹H-NMR (300 MHz, DMSO-d₆): δ [ppm]=1.69 (d, 3H), 2.31 (s, 3H), 3.85 (d, 6H), 6.27 (m, 1H), 6.99 (s, 1H), 7.71 (s, 1H), 7.82-7.91 (m, 2H), 7.97 (d, 1H), 8.24 (d, 1H), 8.39 (d, 1H).

### Example 334

### N-{(1R)-1-[3-(3,5-dimethyl-1H-pyrazol-4-yl)phenyl]ethyl}-6,7-dimethoxy-2-methylquinazolin-4-amine

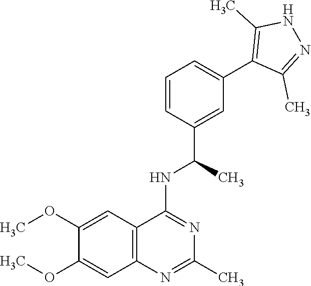 [SMILES: COc1cc2nc(C)nc(N[C@H](C)c3cccc(-c4c(C)n[nH]c4C)c3)c2cc1OC]

### Step a

### N-[(1R)-1-(3-bromophenyl)ethyl]-6,7-dimethoxy-2-methylquinazolin-4-amine

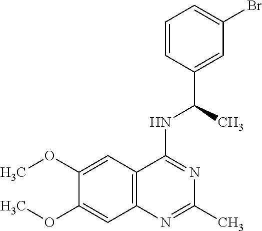 [SMILES: COc1cc2nc(C)nc(N[C@H](C)c3cccc(Br)c3)c2cc1OC]

4-Chloro-6,7-dimethoxy-2-methylquinazoline, 1.20 g (5.0 mmol, commercially available) was dissolved in 5 mL of 2-propanol, then (R)-1-(3-bromophenyl)ethanamine, 1.31 g (6.5 mmol, commercially available), was added at room temperature. The resulting mixture was stirred at 110° C. for 6 hours. The reaction was cooled to room temperature and water was added. The resulting solution was extracted with ethyl acetate. The combined organic phase was dried over anhydrous sodium sulfate and the solvent was removed in vacuo. The residue was purified by silica gel column chromatography to give 1.70 g (66%) of the product as a light yellow solid. MS (ESIpos): m/z=402 [M+H]⁺. LC-MS [method 4, gradient starting with Water (0.05% NH₄HCO₃)-Acetonitrile, 5% B]: Rt=1.59 min. 1H-NMR (400 MHz, DMSO-d₆): δ [ppm]=1.55-1.57 (d, 3H), 2.33 (s, 3H), 3.85 (s, 3H), 3.90 (s, 3H), 5.57-5.61 (m, 1H), 7.01 (s, 1H), 7.26-7.30 (m, 1H), 7.39-7.44 (m, 2H), 7.61-7.62 (d, 1H), 7.67 (s, 1H), 7.97-7.99 (m, 1H).

### Step b

### N-{(1R)-1-[3-(3,5-dimethyl-1H-pyrazol-4-yl)phenyl]ethyl}-6,7-dimethoxy-2-methylquinazolin-4-amine

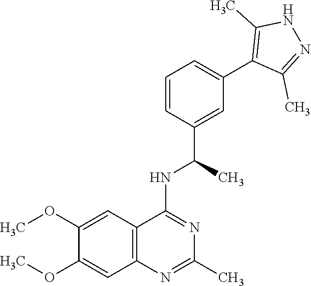 [SMILES: COc1cc2nc(C)nc(N[C@H](C)c3cccc(-c4c(C)n[nH]c4C)c3)c2cc1OC]

This compound was synthesized by the same method as described in example 335 to give 37.4 mg (30%) of the product as a yellow solid. MS (ESIpos): m/z=418 [M+H]⁺; LC-MS [method 4, gradient starting with Water (0.05% TFA)-Acetonitrile, 5% B]: Rt=1.01 min. ¹H-NMR (400 MHz, DMSO-d₆): δ [ppm]=1.63 (d, 3H), 2.14 (s, 6H), 2.35 (s, 3H), 3.88 (d, 6H), 5.71 (m, 1H), 7.03 (d, 1H), 7.10-7.17 (m, 1H), 7.35 (m, 3H), 7.71 (s, 1H), 7.99 (d, 1H), 12.23 (s, 1H).

### Example 335

### 6,7-dimethoxy-2-methyl-N-{(1R)-1-[3-(5-methyl-1H-pyrazol-4-yl)phenyl]ethyl}quinazolin-4-amine

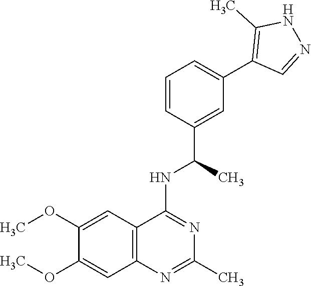 [SMILES: COc1cc2nc(C)nc(N[C@H](C)c3cccc(-c4cn[nH]c4C)c3)c2cc1OC]

(R)—N-(1-(3-bromophenyl)ethyl)-6,7-dimethoxy-2-methylquinazolin-4-amine (described in example 191), 120 mg (0.30 mmol), 5-methyl-4-(4,4,5,5-tetramethyl-1,3,2-dioxaborolan-2-yl)-1H-pyrazole, 186 mg (0.90 mmol), cesium carbonate, 486 mg (1.49 mmol), and tetrakis(triphenylphosphine)palladium(0), 34 mg (0.03 mmol), were dissolved in 2 mL of N,N-dimethylformamide/water (5:2). The resulting mixture was stirred at 80° C. for overnight under nitrogen atmosphere. The reaction was cooled to room temperature and water was added. The resulting solution was extracted with ethyl acetate. The combined organic phase was dried over anhydrous sodium sulfate and the solvent was removed in vacuo. The residue was purified by preparative HPLC to give 43.4 mg (36%) of the product as a white solid. MS (ESIpos): m/z=404 [M+H]⁺; LC-MS [method 4, gradient starting with Water (0.05% TFA)-Acetonitrile, 5% B]: Rt=1.06 min. ¹H-NMR (400 MHz, DMSO-d₆): δ [ppm]=1.62 (d, 3H), 2.29 (s, 3H), 2.32 (s, 3H), 3.88 (d, 6H), 5.71 (m, 1H), 7.03 (d, 1H), 7.31 (m, 3H), 7.52 (s, 1H), 7.71 (s, 2H), 7.99 (d, 1H), 12.59 (s, 1H).

### Example 336

### N-{(1R)-1-[3-(3,5-dimethyl-1,2-oxazol-4-yl)phenyl]ethyl}-6,7-dimethoxy-2-methyl-quinazolin-4-amine

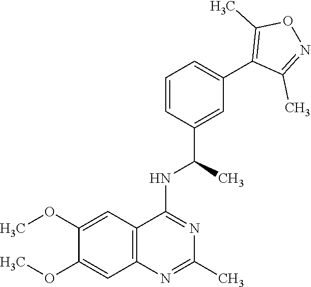 [SMILES: COc1cc2nc(C)nc(N[C@H](C)c3cccc(-c4c(C)noc4C)c3)c2cc1OC]

This compound was synthesized by the same method as described in example 338 to give 35.2 mg (27%) of the product as an off-white solid. MS (ESIpos): m/z=419 [M+H]⁺; LC-MS [method 4, gradient starting with Water (0.05% TFA)-Acetonitrile, 5% B]: Rt=2.66 min. ¹H-NMR (300 MHz, DMSO-d₆): δ [ppm]=1.59 (d, 3H), 2.14 (s, 3H), 2.31 (d, 6H), 3.84 (d, 6H), 5.64 (m, 1H), 6.98 (s, 1H), 7.20 (d, 1H), 7.32-7.47 (m, 3H), 7.66 (s, 1H), 7.97 (d, 1H).

### Example 337

### 6,7-dimethoxy-2-methyl-N-{(1R)-1-[3-(1H-pyrazol-5-yl)phenyl]ethyl}quinazolin-4-amine

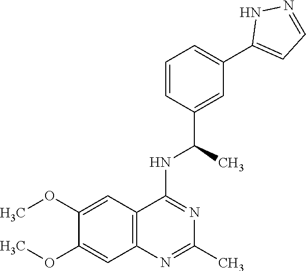 [SMILES: COc1cc2nc(C)nc(N[C@H](C)c3cccc(-c4ccn[nH]4)c3)c2cc1OC]

This compound was synthesized by the same method as described in example 338 to give 52.3 mg (43%) of the product as a white solid. MS (ESIpos): m/z=390 [M+H]⁺; LC-MS [method 4, gradient starting with Water (0.05% TFA)-Acetonitrile, 5% B]: Rt=2.70 min. ¹H-NMR (300 MHz, DMSO-d₆): δ [ppm]=1.59 (d, 3H), 2.31 (s, 3H), 3.84 (d, 6H), 5.66 (m, 1H), 6.62 (s, 1H), 6.98 (s, 1H), 7.31 (m, 2H), 7.50-8.08 (m, 5H), 12.80-13.48 (m, 1H).

### Example 338

### 6,7-dimethoxy-2-methyl-N-{(1R)-1-[3-(1-methyl-1H-pyrazol-4-yl)phenyl]ethyl}quinazolin-4-amine

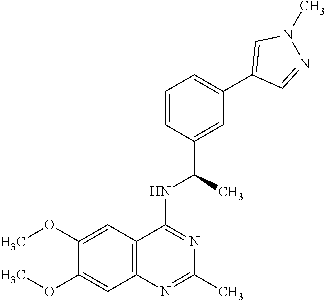

(R)—N-(1-(3-bromophenyl)ethyl)-6,7-dimethoxy-2-methylquinazolin-4-amine (described in example 191), 120 mg (0.30 mmol), 3,5-dimethylisoxazol-4-ylboronic acid, 310 mg (1.49 mmol), potassium carbonate, 206 mg (1.49 mmol), and 1,1′-Bis(diphenylphosphino)ferrocenepalladium(II) chloride, 44 mg (0.06 mmol), were dissolved in 2 mL of N,N-dimethylformamide. The resulting mixture was stirred at 80° C. for overnight under nitrogen atmosphere. The reaction was cooled to room temperature and water was added. The resulting solution was extracted with ethyl acetate. The combined organic phase was dried over anhydrous sodium sulfate and the solvent was removed in vacuo. The residue was purified by preparative HPLC to give 45.5 mg (37%) of the product as a grey solid. MS (ESIpos): m/z=404 [M+H]⁺; LC-MS [method 4, gradient starting with Water (0.05% TFA)-Acetonitrile, 5% B]: Rt=1.13 min. ¹H-NMR (400 MHz, DMSO-d₆): δ [ppm]=1.60 (d, 3H), 2.36 (s, 3H), 3.81-3.94 (m, 9H), 5.63-5.71 (m, 1H), 7.01 (s, 1H), 7.22-7.33 (m, 2H), 7.39 (d, 1H), 7.48-7.62 (m, 2H), 7.72 (s, 1H), 7.80 (s, 1H), 8.08 (s, 1H).

### Example 339

### 6,7-dimethoxy-2-methyl-N-{(1R)-1-[3-(1-methyl-1H-pyrazol-5-yl)phenyl]ethyl}quinazolin-4-amine

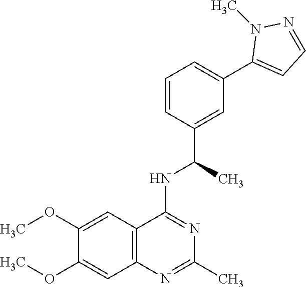 [SMILES: COc1cc2nc(C)nc(N[C@H](C)c3cccc(-c4ccnn4C)c3)c2cc1OC]

This compound was synthesized by the same method as described in example 338 to give 52.3 mg (43%) of the product as a white solid. MS (ESIpos): m/z=404 [M+H]⁺; LC-MS [method 4, gradient starting with Water (0.05% TFA)-Acetonitrile, 5% B]: Rt=1.13 min. ¹H-NMR (300 MHz, DMSO-d₆): δ [ppm]=1.59 (d, 3H), 2.31 (s, 3H), 3.73-3.90 (m, 9H), 5.66 (m, 1H), 6.33 (d, 1H), 6.98 (s, 1H), 7.29-7.58 (m, 5H), 7.66 (s, 1H), 7.99 (d, 1H),

### Example 340

### N-{(1R)-1-[3-(1H-imidazol-1-yl)phenyl]ethyl}-6,7-dimethoxy-2-methylquinazolin-4-amine

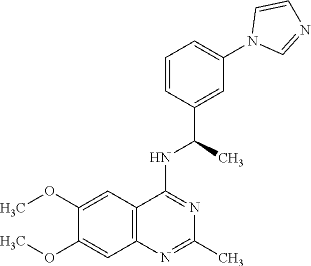 [SMILES: COc1cc2nc(C)nc(N[C@H](C)c3cccc(-n4ccnc4)c3)c2cc1OC]

This compound was synthesized by the same method as described in example 341 to give 52.7 mg (36%) of the product as a white solid. MS (ESIpos): m/z=390 [M+H]⁺; LC-MS [method 4, gradient starting with Water (0.05% TFA)-Acetonitrile, 5% B]: Rt=0.83 min. ¹H-NMR (300 MHz, CD₃OD): δ [ppm]=1.60 (d, 3H), 2.31 (s, 3H), 3.85 (d, 6H), 5.66 (m, 1H), 6.99 (s, 1H), 7.03-7.11 (m, 1H), 7.34-7.51 (m, 3H), 7.63-7.71 (m, 3H), 7.96 (d, 1H), 8.19 (d, 1H).

### Example 341

### 6,7-dimethoxy-2-methyl-N-{(1R)-1-[3-(1H-pyrazol-1-yl)phenyl]ethyl}quinazolin-4-amine

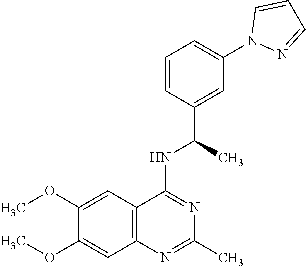 [SMILES: COc1cc2nc(C)nc(N[C@H](C)c3cccc(-n4cccn4)c3)c2cc1OC]

(R)—N-(1-(3-bromophenyl)ethyl)-6,7-dimethoxy-2-methylquinazolin-4-amine (described in example 191), 150 mg (0.37 mmol), 1H-pyrazole, 127 mg (1.86 mmol), copper (I) iodide, 7 mg (0.04 mmol), potassium carbonate, 258 mg (1.86 mmol), and (S)-(−)-proline, 9 mg (0.08 mmol), were dissolved in 2 mL of dimethyl sulfoxide. The resulting mixture was stirred at 80° C. for 4 days under nitrogen atmosphere. The reaction was cooled to room temperature and water was added. The resulting solution was extracted with ethyl acetate. The combined organic phase was dried over anhydrous sodium sulfate and the solvent was removed in vacuo. The residue was purified by preparative HPLC to give 7.8 mg (5%) of the product as a colorless oil. MS (ESIpos): m/z=390 [M+H]⁺; LC-MS [method 4, gradient starting with Water (0.05% TFA)-Acetonitrile, 5% B]: Rt=1.17 min. ¹H-NMR (300 MHz, CD₃OD): δ [ppm]=1.68 (d, 3H), 2.41 (s, 3H), 3.93 (d, 6H) 5.72 (m, 1H), 6.48 (m, 1H), 6.99 (s, 1H), 7.37-7.45 (m, 2H), 7.47-7.59 (m, 1H), 7.59-7.71 (m, 2H), 7.82 (s, 1H), 8.15 (d, 1H).

### Example 342

### N-{(1R)-1-[3-(1H-imidazol-4-yl)phenyl]ethyl}-6,7-dimethoxy-2-methylquinazolin-4-amine

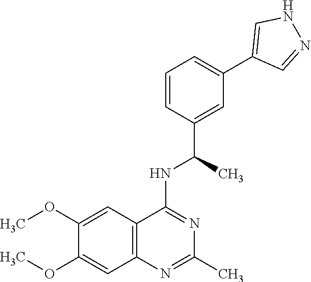 [SMILES: COc1cc2nc(C)nc(N[C@H](C)c3cccc(-c4cn[nH]c4)c3)c2cc1OC]

### Step a

### 5-(4,4,5,5-tetramethyl-1,3,2-dioxaborolan-2-yl)-1H-imidazole

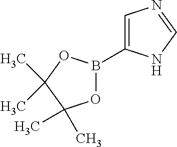

5-iodo-1H-imidazole, 200 mg (1.03 mmol), 4,4,4′,4′,5,5,5′,5′-octamethyl-2,2′-bi(1,3,2-dioxaborolane), 524 mg (2.06 mmol), potassium acetate, 304 mg (3.09 mmol), and 1,1′-bis(diphenylphosphino)ferrocenepalladium(II)chloride, 75 mg (0.10 mmol), were dissolved in 5 mL of N,N-dimethylformamide. The resulting mixture was stirred at 90° C. for overnight under nitrogen atmosphere. The reaction was cooled to room temperature and water was added. The resulting solution was extracted with ethyl acetate. The combined organic phase was dried over anhydrous sodium sulfate and the solvent was removed in vacuo to give 600 mg (crude) of the product as a grey solid and it was used directly for next step. MS (ESIpos): m/z=195 [M+H]⁺; LC-MS [method 4, gradient starting with Water (0.05% TFA)-Acetonitrile, 5% B]: Rt=1.53 min.

### Step b

### N-{(1R)-1-[3-(1H-imidazol-4-yl)phenyl]ethyl}-6,7-dimethoxy-2-methylquinazolin-4-amine

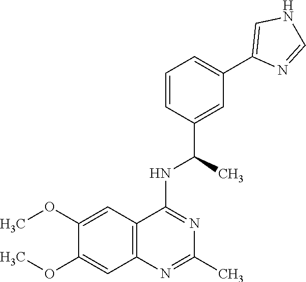 [SMILES: COc1cc2nc(C)nc(N[C@H](C)c3cccc(-c4c[nH]cn4)c3)c2cc1OC]

This compound was synthesized by the same method as described in example 335 to give 3.6 mg (3%) of the product as a white solid. MS (ESIpos): m/z=390 [M+H]⁺; LC-MS [method 4, gradient starting with Water (0.05% TFA)-Acetonitrile, 5% B]: Rt=1.07 min. ¹H-NMR (300 MHz, CD3OD): δ [ppm]=1.70 (d, 3H), 2.50 (s, 3H), 3.95 (d, 6H), 5.76 (m, 1H), 7.00 (s, 1H), 7.25-7.34 (m, 2H), 7.38-7.48 (m, 1H), 7.64 (m, 1H), 7.72 (s, 1H), 7.90 (s, 2H), 8.49 (s, 1H).

### Example 343

### 6-(benzyloxy)-N-[(1R)-1-(3-bromophenyl)ethyl]-7-methoxy-2-methylquinazolin-4-amine

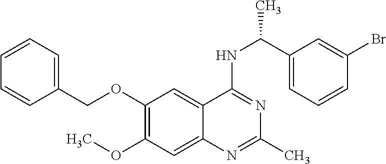 [SMILES: COc1cc2nc(C)nc(N[C@H](C)c3cccc(Br)c3)c2cc1OCc1ccccc1]

### Step a

### methyl 5-(benzyloxy)-4-methoxy-2-aminobenzoate

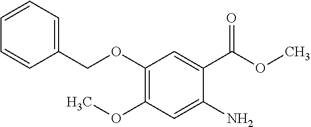 [SMILES: COC(=O)c1cc(OCc2ccccc2)c(OC)cc1N]

Methyl 5-(benzyloxy)-4-methoxy-2-nitrobenzoate, 3.00 g (9.5 mmol), and iron powder, 5.28 g (94.5 mmol), were added into 70 mL of ethanol. Then ammoniumchloride, 5.06 (94.5 mmol), in 15 mL of water was added dropwise at 90° C. and the resulting mixture was stirred at this temperature for 4 hours. After cooling to room temperature, excess iron powder was removed by filtration. The filtrate was concentrated under vacuum and the residue was purified by silica gel column chromatography to give 2.50 g (91%) of the product as a light yellow solid. MS (ESIpos): m/z=287 [M+H]⁺. LC-MS [method 4, Water (0.05% TFA)-Acetonitrile, 5% B]: Rt=1.01 min.

### Step b

### 6-(benzyl oxy)-7-methoxy-2-methylquinazolin-4(3H)-one

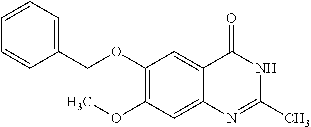 [SMILES: COc1cc2nc(C)[nH]c(=O)c2cc1OCc1ccccc1]

Dry hydrochloric acid gas was passed (until the clear solution observed) to a solution of 5-bromo-2,4-dichloro-7-(phenylsulfonyl)-7H-pyrrolo[2,3-d]pyrimidine, 2.00 g (6.9 mmol) in 50 mL of acetonitrile for 2 hours at room temperature. The precipitated solid was collected by filtration and the filter cake was dissolved in water. The solution was neutralized with 10% aqueous sodium bicarbonate and the precipitated solid was collected by filtration, washed with ice cold water and dried in oven to give 1.0 g (71%) of the product as an off-white solid. MS (ESIpos): m/z=297 [M+H]⁺. LC-MS [method 4, gradient starting with Water (0.05% TFA)-Acetonitrile, 5% B]: Rt=1.06 min. ¹H-NMR (400 MHz, DMSO-d₆): δ [ppm]=2.31 (s, 3H), 3.89 (s, 3H), 5.19 (s, 2H), 7.08 (s, 1H), 7.33-7.50 (m, 6H), 12.02 (s, 1H).

### Step c

### 6-(benzyloxy)-4-chloro-7-methoxy-2-methylquinazoline

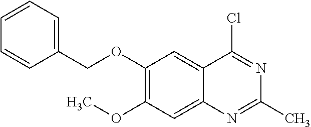 [SMILES: COc1cc2nc(C)nc(Cl)c2cc1OCc1ccccc1]

6-(Benzyloxy)-7-methoxy-2-methylquinazolin-4(3H)-one, 1.45 g (4.9 mmol), was added to 13 mL of thionyl chloride at 0° C. Then 1.0 mL of N,N-dimethylformamide was added at room temperature. The resulting mixture was stirred at reflux for 10 hours. After cooling to room temperature, the mixture was poured into ice water. Aqueous sodium bicarbonate was added to pH=7 and the resulting solution was extracted with dichloromethane. The combined organic phase was dried over anhydrous sodium sulfate and the solvent was removed in vacuo to give 900 mg (54%) of the product as a light brown solid. MS (ESIpos): m/z=315 [M+H]⁺. LC-MS [method 4, gradient starting with Water (0.05% TFA)-Acetonitrile, 5% B]: Rt=1.05 min.

### Step d

### 6-(benzyloxy)-N-[(1R)-1-(3-bromophenyl)ethyl]-7-methoxy-2-methylquinazolin-4-amine

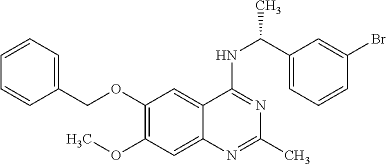 [SMILES: COc1cc2nc(C)nc(N[C@H](C)c3cccc(Br)c3)c2cc1OCc1ccccc1]

6-(Benzyloxy)-4-chloro-7-methoxy-2-methylquinazoline, 350 mg (1.0 mmol), and (R)-1-(3-bromophenyl)ethanamine, 271 mg (1.4 mmol), were dissolved in 5 mL of 2-propanol. The resulting mixture was stirred at 110° C. for 6 hours. After cooling to room temperature, the solvent was removed in vacuo and the residue was purified by silica gel column chromatography to give 490 mg (98%) of the product as a light brown solid. MS (ESIpos): m/z=478 [M+H]⁺. LC-MS [method 4, gradient starting with Water (0.05% TFA)-Acetonitrile, 5% B]: Rt=1.51 min. ¹H-NMR (400 MHz, DMSO-d₆): δ [ppm]=1.56 (s, 3H), 2.34 (s, 3H), 3.85 (s, 3H), 5.16-5.20 (m, 2H), 5.60-5.62 (m, 1H), 7.04 (s, 1H), 7.26-7.28 (m, 1H), 7.30-7.42 (m, 5H), 7.44-7.45 (m, 2H), 7.55 (s, 1H), 7.99 (s, 1H), 8.01 (m, 1H).

### Example 344

### 6-(benzyloxy)-7-methoxy-2-methyl-N-{(1R)-1-[3-(1H-pyrazol-4-yl)phenyl]ethyl}-quinazolin-4-amine

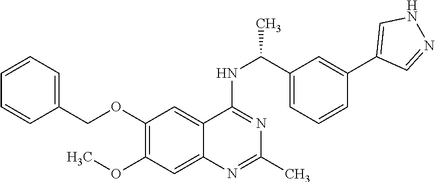 [SMILES: COc1cc2nc(C)nc(N[C@H](C)c3cccc(-c4cn[nH]c4)c3)c2cc1OCc1ccccc1]

6-(Benzyloxy)-N-[(1R)-1-(3-bromophenyl)ethyl]-7-methoxy-2-methylquinazolin-4-amine (described in example 343), 300 mg (0.6 mmol), tert-butyl 4-(4,4,5,5-tetramethyl-1,3,2-dioxaborolan-2-yl)-1H-pyrazole-1-carboxylate, 518 mg (1.8 mmol), tetrakis(triphenylphosphine)palladium(0), 68 mg (0.1 mmol) and cesium carbonate, 574 mg (1.8 mmol), were added into 10 mL of N,N-dimethylformamide and 4 mL of water. The resulting mixture was stirred at 80° C. for 16 hours under nitrogen atmosphere. After cooling to room temperature, water was added and the resulting solution was extracted with ethyl acetate. The solvent was removed in vacuo and the residue was purified by silica gel column chromatography to give 240 mg (87%) of the product as a yellow solid. MS (ESIpos): m/z=466 [M+H]⁺. LC-MS [method 4, gradient starting with Water (0.05% TFA)-Acetonitrile, 5% B]: Rt=1.29 min. ¹H-NMR (400 MHz, DMSO-d₆): δ [ppm]=1.60-1.62 (d, 3H), 2.35 (s, 3H), 3.85 (s, 3H), 5.16-5.17 (s, 2H), 5.66-5.69 (m, 1H), 7.03 (s, 1H), 7.24-7.29 (m, 2H), 7.30-7.45 (m, 4H), 7.52-7.54 (m, 2H), 7.68 (s, 1H), 7.89 (s, 2H), 7.96-7.98 (m, 1H), 8.14 (s, 1H), 12.92 (s, 1H).

### Example 345

### 7-methoxy-2-methyl-4-({(1R)-1-[3-(1H-pyrazol-4-yl)phenyl]ethyl}amino)quinazolin-6-ol

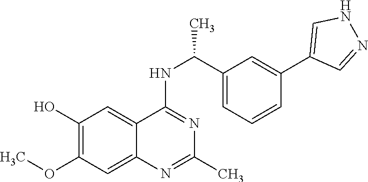 [SMILES: COc1cc2nc(C)nc(N[C@H](C)c3cccc(-c4cn[nH]c4)c3)c2cc1O]

6-(Benzyloxy)-7-methoxy-2-methyl-N-{(1R)-1-[3-(1H-pyrazol-4-yl)phenyl]ethyl}quinazolin-4-amine (described in example 344), 800 mg (1.6 mmol), 10% palladium carbon, 80 mg, and 0.5 mL of hydrochloric acid (2.0 M), were added into 20 mL of methanol. The resulting mixture was stirred at room temperature for 13 hours under hydrogen (2 atm) atmosphere. Then palladium carbon was filtered out and washed with methanol. The filtrate was concentrated in vacuo to give 580 mg (89%) of the product as a light brown solid. MS (ESIpos): m/z=376 [M+H]⁺. LC-MS [method 4, gradient starting with Water (0.05% TFA)-Acetonitrile, 5% B]: Rt=2.41 min. ¹H-NMR (400 MHz, DMSO-d₆): δ [ppm]=1.56-1.58 (d, 3H), 2.34 (s, 3H), 3.90 (s, 3H), 5.57-5.64 (m, 1H), 7.01 (s, 1H), 7.26-7.29 (s, 2H), 7.42-7.43 (s, 1H), 7.66-7.69 (m, 2H), 7.82-7.84 (m, 1H), 8.00 (s, 2H), 8.18 (s, 1H).

### Example 346

### 6-(cyclopropylmethoxy)-7-methoxy-2-methyl-N-{(1R)-1-[3-(1H-pyrazol-4-yl)phenyl]ethyl}quinazolin-4-amine

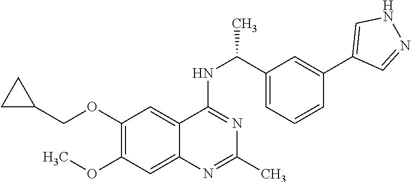 [SMILES: COc1cc2nc(C)nc(N[C@H](C)c3cccc(-c4cn[nH]c4)c3)c2cc1OCC1CC1]

This compound was synthesized by the same method as described in example 355 to give 13.3 mg of the product as an off-white solid. MS (ESIpos): m/z=430 [M+H]⁺. LC-MS [method 4, gradient starting with Water (0.05% TFA)-Acetonitrile, 5% B]: Rt=1.21 min. ¹H-NMR (400 MHz, DMSO-d₆): δ [ppm]=0.32-0.35 (m, 2H), 0.62-0.64 (m, 2H), 1.29-1.34 (m, 1H), 1.60-1.62 (d, 3H), 2.35 (s, 3H), 3.87 (s, 3H), 3.93-3.95 (m, 2H), 5.66-5.71 (m, 1H), 7.01 (s, 1H), 7.24 7.31 (m, 2H), 7.43-7.45 (d, 1H), 7.66-7.67 (d, 2H), 7.87-7.93 (m, 2H), 8.13 (br, 1H), 12.92 (br, 1H).

### Example 347

### 6,7-dimethoxy-2-methyl-N-[1-(thiophen-2-yl)ethyl]quinazolin-4-amine

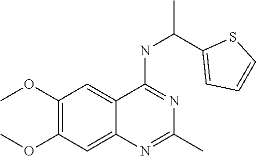 [SMILES: COc1cc2nc(C)nc(NC(C)c3cccs3)c2cc1OC]

N-[1-(5-bromo-2-thienyl)ethyl]-6,7-dimethoxy-2-methylquinazolin-4-amine (described in example 209, 200 mg, 0.5 mmol), was dissolved in 10 mL of tetrahydrofuran, n-butyllithium, 0.43 mL (1.1 mmol), was added to the above solution at −78° C. and the resulting mixture was stirred at this temperature for 30 min under nitrogen atmosphere. Then tri-isopropoxyborane, 181 mg (1.0 mmol), was added at −78° C. the resulting mixture was stirred at this temperature for 1 hour under nitrogen atmosphere. Aq. ammonium chloride was added and the organic solvent was removed in vacuo. 3.0 M hydrogen chloride was added to adjust the pH value to 3 and the resulting mixture was extracted with ethyl acetate. The combined organic layers were dried over anhydrous sodium sulfate and the solvent was removed in vacuo. The residue was purified by preparative HPLC to give 39 mg of a product B as an off-white solid. For B: MS (ESIpos): m/z=330 [M+H]⁺. LC-MS [method 4, gradient starting with Water (0.05% TFA)-Acetonitrile, 5% B]: Rₜ=1.13 min. ¹H-NMR (400 MHz, DMSO-d₆): δ [ppm]=1.67-1.69 (d, 3H), 2.33 (s, 3H), 3.87 (s, 6H), 5.90-5.97 (m, 1H), 6.96-6.97 (m, 1H), 6.98 (s, 1H), 7.04-7.08 (m, 1H), 7.35-7.36 (d, 1H), 7.63 (s, 1H), 8.06-8.08 (d, 1H).

### Example 348

### 7-methoxy-6-(2-methoxyethoxy)-2-methyl-N-{(1R)-1-[3-(1H-pyrazol-4-yl)phenyl]-ethyl}quinazolin-4-amine

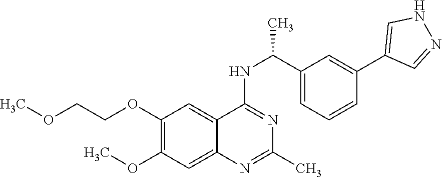 [SMILES: COCCOc1cc2c(N[C@H](C)c3cccc(-c4cn[nH]c4)c3)nc(C)nc2cc1OC]

This compound was synthesized by the same method as described in example 355 to give 5.4 mg of the product as a brown solid. MS (ESIpos): m/z=434 [M+H]⁺. LC-MS [method 4, gradient starting with Water (0.05% TFA)-Acetonitrile, 5% B]: Rt=1.10 min. ¹H-NMR (400 MHz, DMSO-d₆): δ [ppm]=3.75 (s, 3H), 3.74-3.76 (m, 2H), 3.87-3.97 (s, 3H), 4.21-4.22 (m, 2H), 5.66-5.69 (m, 1H), 7.01-7.03 (s, 1H), 7.25-7.31 (m, 3H), 7.44-7.46 (m, 1H), 7.55-7.68 (m, 1H), 7.73-7.89 (s, 1H), 8.09-8.11 (d, 1H), 8.12-8.13 (m, 1H), 12.93 (br, 1H).

### Example 349

### (1R)-1-[2-(5-{1-[(6,7-dimethoxy-2-methylquinazolin-4-yl)amino]ethyl}thiophen-2-yl)phenyl]propan-1-ol

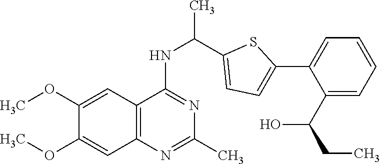 [SMILES: CC[C@@H](O)c1ccccc1-c1ccc(C(C)Nc2nc(C)nc3cc(OC)c(OC)cc23)s1]

To a solution of 2-(5-{1-[(6,7-dimethoxy-2-methylquinazolin-4-yl)amino]ethyl}thiophen-2-yl)benzaldehyde (described in example 179a) 50.0 mg, 115 μmol) in anhydrous THE (1.0 mL) was added chloro(ethyl)magnesium (130 μL, 2.0 M, 250 μmol) dropwise and the reaction mixture stirred during 30 minutes at room temperature. The reaction was quenched with H₂O, the mixture extracted with DCM and the solvent removed in vacuo. Purification by preparative HPLC (basic conditions) gave the title compound as a white solid (27.6 mg, 52%). ¹H-NMR (400 MHz, DMSO-d₆): δ [ppm]=8.15 (dd, 1H), 7.65 (s, 1H), 7.56 (d, 1H), 7.38-7.33 (m, 1H), 7.26-7.22 (m, 2H), 7.08 (dd, 1H), 7.05 (d, 1H), 6.92 (dd, 1H), 5.95 (quin, 1H), 5.05 (d, 1H), 4.76-4.66 (m, 1H), 3.87 (s, 6H), 2.43 (s, 3H), 1.72 (d, 3H), 1.61-1.47 (m, 2H), 0.82-0.73 (m, 3H). LC-MS (method 7): m/z: [M+H]⁺=464, Rt=0.93 min.

### Example 350

### 6-butoxy-7-methoxy-2-methyl-N-{(1R)-1-[3-(1H-pyrazol-4-yl)phenyl]ethyl}quinazolin-4-amine

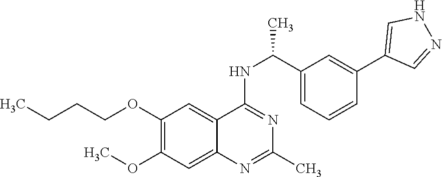 [SMILES: CCCCOc1cc2c(N[C@H](C)c3cccc(-c4cn[nH]c4)c3)nc(C)nc2cc1OC]

This compound was synthesized by the same method as described in example 355 to give 3.3 mg of the product as an off-white solid. MS (ESIpos): m/z=432 [M+H]⁺. LC-MS [method 4, gradient starting with Water (0.05% TFA)-Acetonitrile, 5% B]: Rt=1.27 min. ¹H-NMR (400 MHz, DMSO-d₆): δ [ppm]=0.94-0.97 (m, 3H), 1.43-1.46 (m, 2H), 1.51-1.55 (d, 3H), 1.71-1.80 (m, 2H), 2.31-2.35 (s, 3H), 3.81-3.83 (s, 3H), 4.06-4.10 (m, 2H), 5.65-5.68 (m, 1H), 7.02 (s, 1H), 7.23-7.26 (m, 2H), 7.43-7.46 (m, 1H), 7.65-7.69 (m, 2H), 7.83-7.86 (s, 1H), 7.92-7.95 (d, 1H), 8.11-8.15 (s, 1H), 12.89-12.93 (br, 1H).

### Example 351

### 7-methoxy-2-methyl-6-(3-methylbutoxy)-N-{(1R)-1-[3-(1H-pyrazol-4-yl)phenyl]ethyl}quinazolin-4-amine

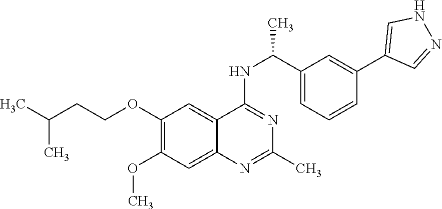 [SMILES: COc1cc2nc(C)nc(N[C@H](C)c3cccc(-c4cn[nH]c4)c3)c2cc1OCCC(C)C]

This compound was synthesized by the same method as described in example 355 to give 4.3 mg of the product as a light yellow solid. MS (ESIpos): m/z=446 [M+H]⁺. LC-MS [method 4, gradient starting with Water (0.05% TFA)-Acetonitrile, 5% B]: Rt=1.35 min. ¹H-NMR (400 MHz, DMSO-d₆): δ [ppm]=0.96-0.98 (d, 6H), 1.66-1.69 (m, 5H), 1.71-1.73 (m, 1H), 2.33 (s, 3H), 3.90 (s, 3H), 4.12-4.13 (m, 2H), 5.72-5.76 (m, 1H), 7.04 (s, 1H), 7.26-7.33 (m, 2H), 7.46-7.48 (d, 1H), 7.69 (s, 1H), 7.77-7.79 (s, 1H), 7.81-7.90 (s, 1H), 8.15 (s, 1H), 12.94 (br, 1H).

### Example 352

### tert-butyl {2-[2-(5-{1-[(6,7-dimethoxy-2-methylquinazolin-4-yl)amino]ethyl}thiophen-2-yl)phenyl]ethyl}carbamate

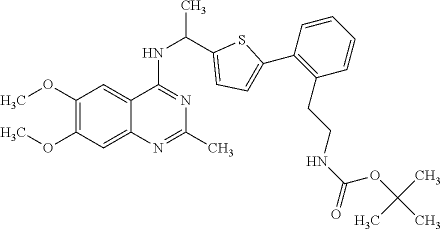 [SMILES: COc1cc2nc(C)nc(NC(C)c3ccc(-c4ccccc4CCNC(=O)OC(C)(C)C)s3)c2cc1OC]

### Step a

### N-[1-(5-bromo-2-thienyl)ethyl]-6,7-dimethoxy-2-methylquinazolin-4-amine

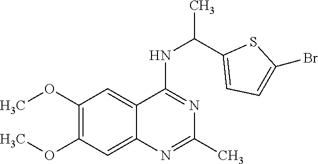 [SMILES: COc1cc2nc(C)nc(NC(C)c3ccc(Br)s3)c2cc1OC]

4-Chloro-6,7-dimethoxy-2-methylquinazoline, 1.50 g (6.3 mmol), and 1-(5-bromothiophen-2-yl) ethanamine, 1.68 g (8.2 mmol), were added into 15 mL of 2-propanol. The resulting mixture was stirred at 110° C. for overnight. After cooled to room temperature, the solvent was removed in vacuo and the residue was purified by silica gel column chromatography to give 1.80 g (70%) of the product as a brown solid. MS (ESIpos): m/z=408 [M+H]⁺. LC-MS [method 4, gradient starting with Water (0.05% NH₄HCO₃)-Acetonitrile, 10% B]: Rₜ=1.12 min.

### Step b

### tert-butyl [2-(2-bromophenyl)ethyl]carbamate

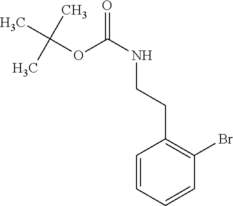 [SMILES: CC(C)(C)OC(=O)NCCc1ccccc1Br]

2-(2-Bromophenyl)ethanamine, 1.00 g (5.0 mmol), di-tert-butyl dicarbonate, 2.18 g (10.0 mmol), and triethylamine, 1.52 g (15.0 mmol), were added into 15 mL of dichloromethane. The resulting mixture was stirred at room temperature for 13 hours. The solvent was removed in vacuo and the residue was purified by silica gel column chromatography to give 1.10 g (73%) of the product as a light yellow solid. MS (ESIpos): m/z=300 [M+H]⁺, LC-MS [method 4, gradient starting with Water (0.05% NH₄HCO₃)-Acetonitrile, 10% B]: Rₜ=1.83 min.

### Step c

### tert-butyl {2-[2-(4,4,5,5-tetramethyl-1,3,2-dioxaborolan-2-yl)phenyl]ethyl}carbamate

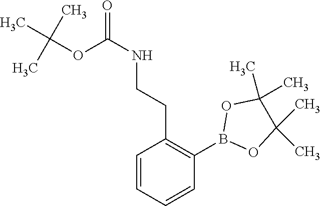 [SMILES: CC(C)(C)OC(=O)NCCc1ccccc1B1OC(C)(C)C(C)(C)O1]

Tert-butyl [2-(2-bromophenyl)ethyl]carbamate, 500 mg (1.7 mmol), was dissolved in 15 mL of 1,4-dioxane, then bis(pinacolato) diboron 1.69 g (6.7 mmol), potassium acetate, 654 mg (6.7 mmol) and 1,1′-bis(diphenylphosphino)ferrocenepalladiumdichloride CH₂Cl₂, 272 mg (0.3 mmol), were added successively to the above solution. The resulting mixture was stirred at 100° C. for overnight under nitrogen atmosphere. After cooled to room temperature, the solvent was removed in vacuo and water was added. The resulting mixture was extracted with ethyl acetate, washed with water and the combined organic layers were concentrated under vacuum. The residue was purified by silica gel column chromatography to give 370 mg (58%) of the product as a light yellow solid. MS (ESIpos): m/z=348 [M+H]⁺, LC-MS [method 4, gradient starting with Water (0.05% TFA)-Acetonitrile, 5% B]: Rₜ=1.30 min.

### Step d

### tert-butyl {2-[2-(5-{1-[(6,7-dimethoxy-2-methylquinazolin-4-yl)amino]ethyl}thiophen-2-yl)phenyl]ethyl}carbamate

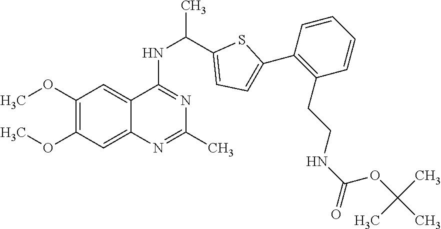 [SMILES: COc1cc2nc(C)nc(NC(C)c3ccc(-c4ccccc4CCNC(=O)OC(C)(C)C)s3)c2cc1OC]

N-[1-(5-bromo-2-thienyl)ethyl]-6,7-dimethoxy-2-methylquinazolin-4-amine, 200 mg (0.5 mmol), tert-butyl {2-[2-(4,4,5,5-tetramethyl-1,3,2-dioxaborolan-2-yl)phenyl]ethyl}carbamate, 370 mg (1.0 mmol), potassium carbonate, 135 mg (1.0 mmol) and tetrakis(triphenylphosphine) palladium(0), 113 mg (0.1 mmol), were added into 12 mL of 1,4-dioxane/water (5:1, v:v). The resulting mixture was stirred at 110° C. for overnight under nitrogen atmosphere. After cooled to room temperature, the solvent was removed in vacuo and water was added. The resulting mixture was extracted with dichloromethane, washed with water and the combine organic layers was concentrated under vacuum. The residue was purified by silica gel column chromatography to give 190 mg (30%) of the product as a light yellow solid. 90 mg of the product was purified by preparative HPLC to give 32.8 mg of the product as an off-white solid. MS (ESIpos): m/z=549 [M+H]⁺, LC-MS [method 4, gradient starting with Water (0.05% TFA)-Acetonitrile, 5% B]: Rₜ=1.50 min. ¹H-NMR (400 MHz, DMSO-d₆): δ [ppm]=1.15-1.34 (s, 9H), 1.71-1.73 (d, 3H), 2.44 (s, 3H), 2.79-2.82 (m, 2H), 3.09-3.14 (m, 2H), 3.87 (s, 6H), 5.95-6.00 (m, 1H), 6.88-6.91 (s, 1H), 6.99-7.00 (s, 1H), 7.05-7.08 (m, 2H), 7.20-7.31 (m, 4H), 7.65 (s, 1H), 8.14-8.19 (m, 1H).

### Example 353

### 7-methoxy-2-methyl-6-(propan-2-yloxy)-N-{(1R)-1-[3-(1H-pyrazol-4-yl)phenyl]ethyl}quinazolin-4-amine

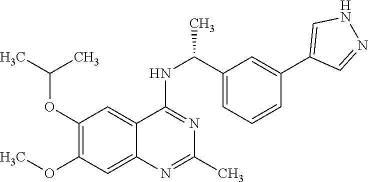 [SMILES: COc1cc2nc(C)nc(N[C@H](C)c3cccc(-c4cn[nH]c4)c3)c2cc1OC(C)C]

This compound was synthesized by the same method as described in example 355 to give 2.9 mg of the product as an off-white solid. MS (ESIpos): m/z=418 [M+H]⁺. LC-MS [method 4, gradient starting with Water (0.05% TFA)-Acetonitrile, 5% B]: Rt=1.18 min. ¹H-NMR (400 MHz, DMSO-d₆): δ [ppm]=1.32-1.33 (m, 6H), 1.60-1.62 (d, 3H), 2.35 (s, 3H), 3.85 (s, 3H), 4.78-4.82 (m, 1H), 5.66-5.70 (s, 1H), 7.02 (s, 1H), 7.24-7.31 (m, 2H), 7.44-7.45 (m, 1H), 7.55-7.60 (m, 2H), 7.68-7.69 (s, 1H), 7.94 (s, 1H), 7.96 (m, 1H), 8.01 (s, 2H), 12.79 (br, 1H).

### Example 354

### 7-methoxy-2-methyl-6-(oxetan-3-ylmethoxy)-N-{(1R)-1-[3-(1H-pyrazol-4-yl)phenyl]ethyl}quinazolin-4-amine

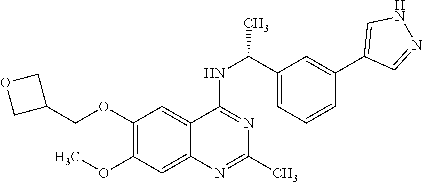 [SMILES: COc1cc2nc(C)nc(N[C@H](C)c3cccc(-c4cn[nH]c4)c3)c2cc1OCC1COC1]

This compound was synthesized by the same method as described in example 355 to give 3.2 mg of the product as a light yellow solid. MS (ESIpos): m/z=446 [M+H]⁺. LC-MS [method 4, gradient starting with Water (0.05% TFA)-Acetonitrile, 5% B]: Rt=1.08 min. ¹H-NMR (400 MHz, DMSO-d₆): δ [ppm]=1.62-1.64 (d, 2H), 2.02-2.04 (m, 1H), 2.33-2.36 (s, 3H), 3.86 (s, 3H), 4.31-4.38 (m, 2H), 4.41-4.48 (m, 2H), 4.71-4.76 (m, 2H), 5.70-5.71 (m, 1H), 7.03 (s, 1H), 7.26 7.32 (m, 2H), 7.44-7.46 (m, 1H), 7.69 (s, 1H), 7.80 (s, 1H), 7.99-8.02 (m, 2H), 8.32 (br, 2H).

### Example 355

### 6-Ethoxy-7-methoxy-2-methyl-N-{(1R)-1-[3-(1H-pyrazol-4-yl)phenyl]ethyl}-quinazolin-4-amine

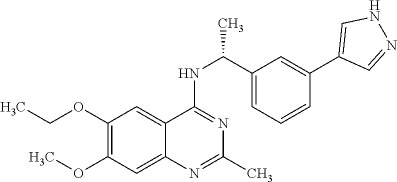 [SMILES: CCOc1cc2c(N[C@H](C)c3cccc(-c4cn[nH]c4)c3)nc(C)nc2cc1OC]

7-Methoxy-2-methyl-4-({(1R)-1-[3-(1H-pyrazol-4-yl)phenyl]ethyl}amino)quinazolin-6-ol (described in example 345), 55 mg (0.14 mmol), potassium carbonate, 29 mg (0.21 mmol), bromoethane, 20 mg (0.18 mmol), and potassium iodide, 5 mg (0.03 mmol), were added into 2 mL of N,N-dimethylformamide. The resulting mixture was stirred at room temperature for 14 hours. The solid was removed by filtration and the filtrate was purified by preparative HPLC to give 1.4 mg of the product as an off-white solid. MS (ESIpos): m/z=404 [M+H]⁺. LC-MS [method 4, gradient starting with Water (0.05% TFA)-Acetonitrile, 5% B]: Rt=1.15 min. ¹H-NMR (400 MHz, DMSO-d₆): δ [ppm]=1.39-1.43 (m, 3H), 1.60-1.62 (m, 3H), 2.34-2.35 (s, 3H), 3.89 (s, 3H), 4.14-4.16 (m, 2H), 5.64-5.71 (m, 1H), 7.01 (s, 1H), 7.25-7.31 (m, 2H), 7.44-7.46 (m, 1H), 7.66-7.72 (d, 2H), 7.97-8.02 (m, 2H), 8.37-8.45 (br, 2H).

### Example 356

### 6-ethoxy-N-{(1R)-1-[3-(1-ethyl-1H-pyrazol-4-yl)phenyl]ethyl}-7-methoxy-2-methylquinazolin-4-amine

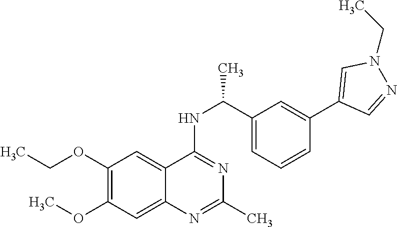 [SMILES: CCOc1cc2c(N[C@H](C)c3cccc(-c4cnn(CC)c4)c3)nc(C)nc2cc1OC]

7-Methoxy-2-methyl-4-({(1R)-1-[3-(1H-pyrazol-4-yl)phenyl]ethyl}amino)quinazolin-6-ol (described in example 345), 55 mg (0.14 mmol), potassium carbonate, 29 mg (0.21 mmol), bromoethane, 20 mg (0.18 mmol), and potassium iodide, 5 mg (0.03 mmol), were added into 2 mL of N,N-dimethylformamide. The resulting mixture was stirred at room temperature for 14 hours. The solid was removed by filtration and the filtrate was purified by preparative HPLC to give 5.0 mg of the product as a light yellow solid. For B: MS (ESIpos): m/z=432 [M+H]⁺. LC-MS [method 4, gradient starting with Water (0.05% TFA)-Acetonitrile, 5% B]: Rₜ=1.25 min. ¹H NMR (400 MHz, DMSO-d₆): δ [ppm]=1.40-1.44 (m, 6H), 1.66-1.68 (d, 3H), 2.51 (s, 3H), 3.92 3.96 (s, 3H), 4.14-4.23 (m, 4H), 5.76-5.79 (m, 1H), 7.08 (s, 1H), 7.26-7.35 (m, 2H), 7.44-7.46 (s, 1H), 7.48-7.68 (s, 1H), 7.70 (s, 1H), 7.85-7.93 (s, 1H), 8.18 (s, 1H), 14.13 (br, 1H).

### Example 357

### N-[1-{5-[2-(2-aminoethyl)phenyl]thiophen-2-yl}ethyl]-6,7-dimethoxy-2-methylquinazolin-4-amine

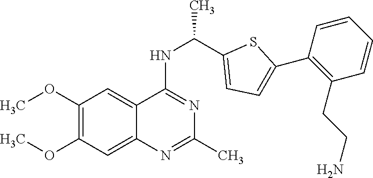 [SMILES: COc1cc2nc(C)nc(N[C@H](C)c3ccc(-c4ccccc4CCN)s3)c2cc1OC]

Tert-butyl{2-[2-(5-{1-[(6,7-dimethoxy-2-methylquinazolin-4-yl)amino]ethyl}-2-thienyl)phenyl]ethyl}carbamate (described in example 352), 50 mg (0.1 mmol), was dissolved in 1.5 mL of 1,4-dioxane, then 1.5 mL of 3.0 M hydrochloric acid/dioxane solution was added into the above solution at 0° C. The resulting mixture was stirred at room temperature for 3 hours. Saturated sodium bicarbonate solution was added to adjust the pH value to 7. The resulting mixture was extracted with ethyl acetate and the combined organic layers were dried over anhydrous sodium sulfate. The solvent removed in vacuo and the residue was purified by preparative HPLC to give 30.1 mg (73%) of the product as an off-white solid. MS (ESIpos): m/z=449 [M+H]⁺, LC-MS [method 4, gradient starting with Water (0.1% HCOOH)-Acetonitrile, 10% B]: Rₜ=0.75 min. ¹H-NMR (400 MHz, DMSO-d₆): δ [ppm]=1.71-1.73 (d, 3H), 2.44 (s, 3H), 2.67-2.84 (m, 4H), 3.12 (br, 2H), 3.87 (s, 6H), 5.93-6.00 (m, 1H), 6.97-7.08 (m, 3H), 7.18-7.31 (m, 4H), 7.65 (s, 1H), 8.16-8.18 (d, 1H).

### Example 358

### tert-butyl {1-[2-(5-{1-[(6,7-dimethoxy-2-methylquinazolin-4-yl)amino]ethyl}thiophen-2-yl)phenyl]ethyl}carbamate

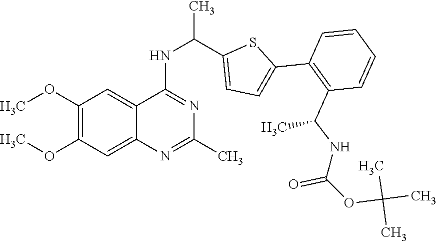 [SMILES: COc1cc2nc(C)nc(NC(C)c3ccc(-c4ccccc4[C@@H](C)NC(=O)OC(C)(C)C)s3)c2cc1OC]

### Step a

### tert-butyl {(1R)-1-[2-(4,4,5,5-tetramethyl-1,3,2-dioxaborolan-2-yl)phenyl]ethyl}carbamate

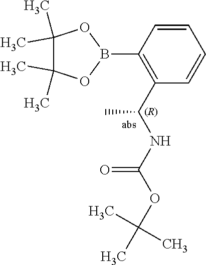

This compound was synthesized by the same method as described in example 352 (step c) to give 800 mg (41%) of the product as a light brown solid. MS (ESIpos): m/z=348 [M+H]⁺, LC-MS [method 4, gradient starting with Water (0.05% TFA)-Acetonitrile, 5% B]: Rₜ=1.56 min.

### Step b

### tert-butyl {1-[2-(5-{1-[(6,7-dimethoxy-2-methylquinazolin-4-yl)amino]ethyl}thiophen-2-yl)phenyl]ethyl}carbamate

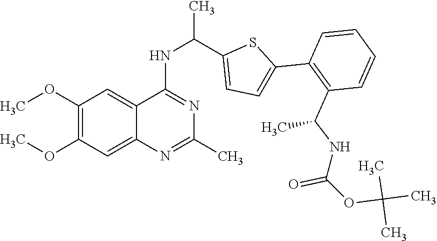 [SMILES: COc1cc2nc(C)nc(NC(C)c3ccc(-c4ccccc4[C@@H](C)NC(=O)OC(C)(C)C)s3)c2cc1OC]

This compound was synthesized by the same method as described in example 352 (step d) to give 7.1 mg of the product as an off-white solid. MS (ESIpos): m/z=549 [M+H]⁺. LC-MS [method 4, gradient starting with water (0.05% TFA)-Acetonitrile, 5% B]: Rₜ=1.48 min. ¹H-NMR (400 MHz, DMSO-d₆): δ [ppm]=1.17-1.23 (d, 3H), 1.32 (s, 9H), 1.72-1.73 (d, 3H), 2.44 (s, 3H), 3.87 (s, 6H), 5.01 (m, 1H), 5.95-5.99 (m, 1H), 7.05-7.10 (m, 3H), 7.21-7.22 (m, 2H), 7.35-7.36 (m, 1H), 7.51-7.53 (d, 2H), 7.65 (s, 1H), 8.21 (d, 1H).

### Example 359

### N-[1-(5-{2-[1-aminoethyl]phenyl}thiophen-2-yl)ethyl]-6,7-dimethoxy-2-methylquinazolin-4-amine

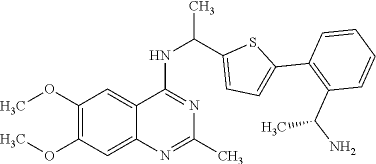 [SMILES: COc1cc2nc(C)nc(NC(C)c3ccc(-c4ccccc4[C@@H](C)N)s3)c2cc1OC]

This compound was synthesized by the same method as described in example 357 to give 6.0 mg (36%) of the product as an off-white solid. MS (ESIpos): m/z=449 [M+H]⁺, LC-MS [method 4, gradient starting with water (0.05% TFA)-Acetonitrile, 5% B]: Rₜ=1.30 min. ¹H-NMR (400 MHz, DMSO-d₆): δ [ppm]=1.32-1.35 (m, 3H), 1.72-1.74 (d, 3H), 2.44 (s, 3H), 3.87 (s, 6H), 4.40-4.41 (s, 1H), 5.93-6.00 (m, 1H), 6.97-6.98 (s, 1H), 7.05 (s, 1H), 7.11 (s, 1H), 7.28-7.32 (m, 2H), 7.41-7.45 (m, 1H), 7.65 (s, 1H), 7.72-7.74 (d, 1H), 8.18-8.20 (d, 1H), 8.32 (br, 2H).

### Example 360

### N-[1-(5-{2-[1-aminoethyl]phenyl}thiophen-2-yl)ethyl]-6,7-dimethoxy-2-methylquinazolin-4-amine

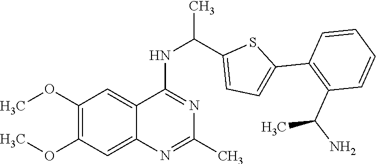 [SMILES: COc1cc2nc(C)nc(NC(C)c3ccc(-c4ccccc4[C@H](C)N)s3)c2cc1OC]

### Step a

### tert-butyl {(1S)-1-[2-(4,4,5,5-tetramethyl-1,3,2-dioxaborolan-2-yl)phenyl]ethyl}carbamate

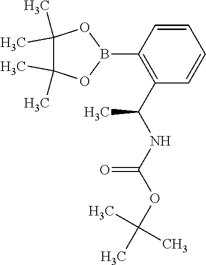 [SMILES: C[C@H](NC(=O)OC(C)(C)C)c1ccccc1B1OC(C)(C)C(C)(C)O1]

This compound was synthesized by the same method as described in example 352 (step c) to give 400 mg (38%) of the product as a light brown solid. MS (ESIpos): m/z=348 [M+H]⁺, LC-MS [method 4, gradient starting with water (0.05% TFA)-Acetonitrile, 5% B]: Rₜ=1.53 min.

### Step b

### tert-butyl rel-{(1R)-1-[2-(5-{(1R)-1-[(6,7-dimethoxy-2-methylquinazolin-4-yl)amino]ethyl}-2-thienyl)phenyl]ethyl}carbamate

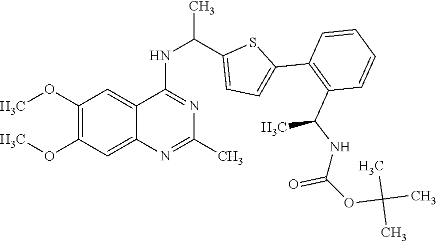 [SMILES: COc1cc2nc(C)nc(NC(C)c3ccc(-c4ccccc4[C@H](C)NC(=O)OC(C)(C)C)s3)c2cc1OC]

This compound was synthesized by the same method as described in example 352 (step d) to give 50 mg (7%) of the product as a light yellow solid. MS (ESIpos): m/z=549 [M+H]⁺. LC-MS [method 4, gradient starting with water (0.05% TFA)-Acetonitrile, 5% B]: Rₜ=1.44 min.

### Step c

### N-[1-(5-{2-[1-aminoethyl]phenyl}thiophen-2-yl)ethyl]-6,7-dimethoxy-2-methylquinazolin-4-amine

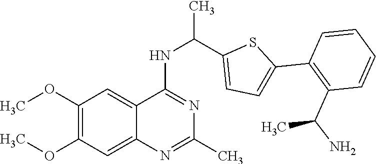 [SMILES: COc1cc2nc(C)nc(NC(C)c3ccc(-c4ccccc4[C@H](C)N)s3)c2cc1OC]

This compound was synthesized by the same method as described in example 357 to give 6.6 mg (60%) of the product as an off-white solid. MS (ESIpos): m/z=449 [M+H]⁺, LC-MS [method 4, gradient starting with water (0.05% TFA)-Acetonitrile, 5% B]: Rₜ=1.30 min. ¹H-NMR (400 MHz, DMSO-d₆): δ [ppm]=1.28-1.39 (d, 3H), 1.72-1.74 (d, 3H), 2.33 (s, 3H), 3.87 (s, 6H), 4.42 (m, 1H), 5.94-5.98 (m, 1H), 6.98 (s, 1H), 7.05 (s, 1H), 7.11 (s, 1H), 7.28-7.33 (m, 2H), 7.42 7.46 (m, 1H), 7.66 (s, 1H), 7.74-7.75 (d, 1H), 8.20-8.23 (d, 1H), 8.25-8.35 (br, 2H).

### Example 361

### 6,7-dimethoxy-2-methyl-N-[1-{5-[2-(1H-pyrazol-4-yl)phenyl]thiophen-2-yl}ethyl]-quinazolin-4-amine

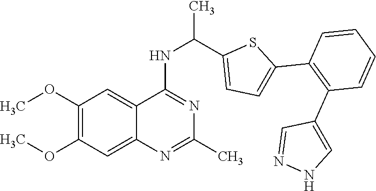 [SMILES: COc1cc2nc(C)nc(NC(C)c3ccc(-c4ccccc4-c4cn[nH]c4)s3)c2cc1OC]

### Step a

### tert-butyl 4-bromo-1H-pyrazole-1-carboxylate

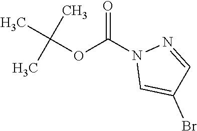

4-Bromo-1H-pyrazole, 5.00 g (34.0 mmol), di-tert-butyl dicarbonate, 8.17 g (37.4 mmol), and triethylamine, 7.57 g (74.8 mmol), were added into 50 mL of dichloromethane. The resulting mixture was stirred at room temperature for overnight. The resulting mixture was purified by silica gel column chromatography to give 8.00 g (93%) of the product as an off-white solid. MS (ESIpos): m/z=247 [M+H]⁺. LC-MS [method 4, gradient starting with water (0.1% HCOOH)-Acetonitrile, 10% B]: Rₜ=1.75 min.

### Step b

### 4-(2-bromophenyl)-1H-pyrazole

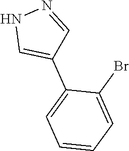 [SMILES: Brc1ccccc1-c1cn[nH]c1]

2-Bromophenylboronic acid, 6.00 g (29.9 mmol), tert-butyl 4-bromo-1H-pyrazole-1-carboxylate, 3.69 g (14.9 mmol), potassium carbonate, 8.26 g (59.8 mmol), and tetrakis(triphenylphosphine) palladium(0), 1.22 g (1.5 mmol), were added into 60 mL of 1,4-dioxane/water (5:1, v:v). The resulting mixture was stirred at 110° C. for overnight under nitrogen atmosphere. After cooled to room temperature, water was added and the resulting mixture was extracted with ethyl acetate and the combined organic layers were dried over anhydrous sodium sulfate. The solvent was removed in vacuo and the residue was purified by silica gel column chromatography to give 2.00 g (26%) of the product as a light yellow solid. MS (ESIpos): m/z=223 [M+H]⁺. LC-MS [method 4, gradient starting with water (0.05% TFA)-Acetonitrile, 5% B]: Rₜ=0.96 min.

### Step c

### tert-butyl 4-(2-bromophenyl)-1H-pyrazole-1-carboxylate

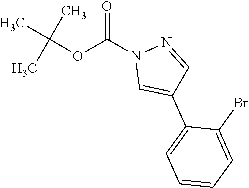 [SMILES: CC(C)(C)OC(=O)n1cc(-c2ccccc2Br)cn1]

4-(2-Bromophenyl)-1H-pyrazole, 2.00 g (7.8 mmol), di-tert-butyl dicarbonate, 1.87 g (8.6 mmol), and triethylamine, 1.74 g (17.2 mmol), were added into 30 mL of dichloromethane. The resulting mixture was stirred at room temperature for overnight. The solvent was removed in vacuo and the residue was purified by silica gel column chromatography to give 1.10 g (54%) of the product as a light yellow oil. MS (ESIpos): m/z=323 [M+H]⁺, LC-MS [method 4, gradient starting with water (0.1% HCOOH)-Acetonitrile, 10% B]: Rₜ=1.27 min.

### Step d

### tert-butyl 4-[2-(4,4,5,5-tetramethyl-1,3,2-dioxaborolan-2-yl)phenyl]-1H-pyrazole-1-carboxylate

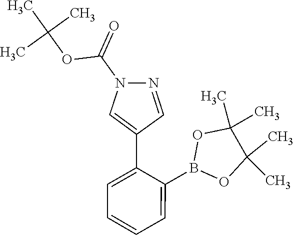 [SMILES: CC(C)(C)OC(=O)n1cc(-c2ccccc2B2OC(C)(C)C(C)(C)O2)cn1]

This compound was synthesized by the same method as described in example 352 (step c) to give 550 mg (52%) of the product as a light yellow oil. MS (ESIpos): m/z=371 [M+H]⁺, LC-MS [method 4, gradient starting with water (0.05% NH₄HCO₃)-Acetonitrile, 10% B]: Rₜ=1.30 min.

### Step e

### 6,7-dimethoxy-2-methyl-N-[1-{5-[2-(1H-pyrazol-4-yl)phenyl]thiophen-2-yl}ethyl]-quinazolin-4-amine

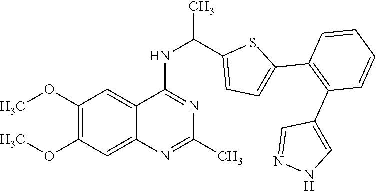 [SMILES: COc1cc2nc(C)nc(NC(C)c3ccc(-c4ccccc4-c4cn[nH]c4)s3)c2cc1OC]

This compound was synthesized by the same method as described in example 352 (step d) to give 73.5 mg (45%) of the product as an off-white solid. MS (ESIpos): m/z=472 [M+H]⁺, LC-MS [method 4, gradient starting with water (0.05% TFA)-Acetonitrile, 5% B]: Rₜ=1.28 min. ¹H-NMR (400 MHz, DMSO-d₆): δ [ppm]=1.66-1.68 (d, 3H), 2.41 (s, 3H), 3.87 (s, 6H), 5.89-5.92 (m, 1H), 6.77-6.78 (s, 1H), 6.97-6.98 (s, 1H), 7.04 (s, 1H), 7.24-7.28 (m, 2H), 7.33-7.36 (m, 2H), 7.44-7.46 (s, 1H), 7.57 (s, 1H), 7.63 (s, 1H), 8.09-8.11 (d, 1H), 12.80 (br, 1H).

### Example 362

### 6,7-dimethoxy-2-methyl-N-[1-(5-{2-[(phenylamino)methyl]phenyl}thiophen-2-yl)ethyl]quinazolin-4-amine

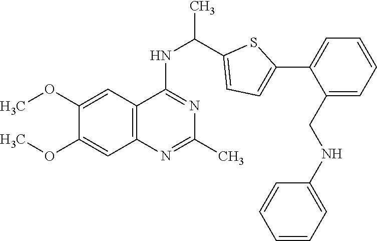 [SMILES: COc1cc2nc(C)nc(NC(C)c3ccc(-c4ccccc4CNc4ccccc4)s3)c2cc1OC]

A mixture of N-[1-(5-Bromothiophen-2-yl)ethyl]-6,7-dimethoxy-2-methylquinazolin-4-amine (prepared as described in example 209; 100 mg, 0.24 mmol), 2-(N-Phenylaminomethyl)phenylboronic acid (commercially available; 58.5 mg, 0.24 mmol), potassium carbonate (135.4 mg, 0.98 mmol) and Tetrakis(triphenylphosphin)palladium(0) (28.3 mg, 0.025 mmol) in dioxane (2.5 mL) and water (0.5 mL) was stirred at 110° C. overnight. The reaction mixture was evaporated and the crude product was purified via HPLC to yield 48% of the title compound (64 mg). ¹H-NMR (400 MHz, DMSO-d₆): δ [ppm]=8.15 (d, 1H), 7.64 (s, 1H), 7.48-7.45 (m, 1H), 7.38-7.26 (m, 3H), 7.12-7.08 (m, 2H), 7.05 (s, 1H), 6.99 (t, 2H), 6.51-6.44 (m, 3H), 6.13 (t, 1H), 5.96 (t, 1H), 4.26 (d, 2H), 3.87 (s, 3H), 3.86 (s, 3H), 2.43 (s, 3H), 1.71 (d, 3H). LC-MS (method 7): m/z: [M+H]⁺=511.2, Rₜ=1.07 min.

### Example 363

### 6-bromo-N-[(1R)-1-(3-chlorophenyl)ethyl]-2-methylquinazolin-4-amine

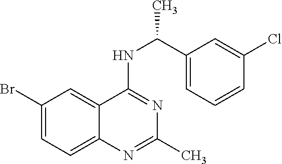 [SMILES: Cc1nc(N[C@H](C)c2cccc(Cl)c2)c2cc(Br)ccc2n1]

A solution of 6-bromo-4-chloro-2-methylquinazoline (commercially available; 5 g, 19.4 mmol), (R)-1-(3-chlorophenyl)ethylamine (3.66 g, 23.3 mmol, commercially available) and N,N-diisopropylethylamine (6.76 mL) in dioxane (75 mL) was stirred overnight at 100° C. After cooling to ambient temperature the reaction mixture was evaporated and the crude material was purified via Isolera flash chromatography (silica gel, gradient hexanes 100% 4 ethylacetate 100%) to obtain the title compound in quantitative yield (7.35 g). ¹H-NMR (400 MHz, DMSO-d₆): δ [ppm]=8.67 (d, 1H), 8.47 (d, 1H), 7.84 (dd, 1H), 7.55-7.48 (m, 2H), 7.42 7.38 (m, 1H), 7.35 (t, 1H), 7.30-7.27 (m, 1H), 5.57 (t, 1H), 2.54-2.52 (m, 1H), 2.38 (s, 3H), 1.57 (d, 3H). LC-MS (method 7): m/z: [M+H]⁺=378.0, Rₜ=0.87 min.

### Example 364

### N-[1-(5-{2-[(cyclopentylamino)methyl]phenyl}thiophen-2-yl)ethyl]-6,7-dimethoxy-2-methylquinazolin-4-amine

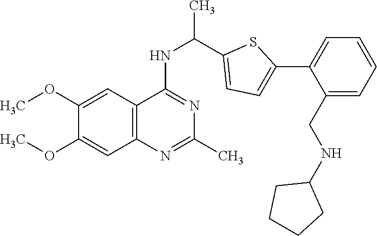 [SMILES: COc1cc2nc(C)nc(NC(C)c3ccc(-c4ccccc4CNC4CCCC4)s3)c2cc1OC]

A mixture of N-[1-(5-bromothiophen-2-yl)ethyl]-6,7-dimethoxy-2-methylquinazolin-4-amine (prepared as described in example 209; 100 mg, 0.24 mmol), 2-(cyclopentylaminomethyl)phenylboronoc acid, pinacol ester (commercially available; 76.8 mg, 0.24 mmol), potassium carbonate (135.4 mg, 0.98 mmol) and tetrakis-(triphenylphosphin)palladium(0) (28.3 mg, 0.025 mmol) in dioxane (2.5 mL) and water (0.5 mL) was stirred at 110° C. overnight. The reaction mixture was evaporated and the crude product was purified via HPLC to yield 49% of the title compound (62 mg). ¹H-NMR (400 MHz, DMSO-d₆): δ [ppm]=8.93 (d, 1H), 8.44 (s, 1H), 8.27 (d, 1H), 8.11-8.01 (m, 3H), 7.97 (d, 1H), 7.87 (d, 1H), 7.84 (s, 1H), 6.75 (t, 1H), 4.46 (s, 2H), 4.12 (s, 6H), 3.76-3.69 (m, 1H), 3.22 (s, 3H), 2.52 (s, 2H), 2.51-2.49 (m, 1H), 2.44-2.28 (m, 4H), 2.22-2.10 (m, 2H), 2.09-1.99 (m, 2H). LC-MS (method 7): m/z: [M+H]⁺=503.3, Rₜ=0.64 min.

### Example 365

### N-[1-(5-{2-[(benzylamino)methyl]phenyl}thiophen-2-yl)ethyl]-6,7-dimethoxy-2-methylquinazolin-4-amine

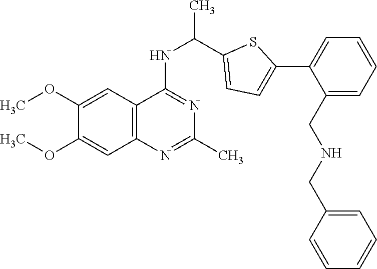 [SMILES: COc1cc2nc(C)nc(NC(C)c3ccc(-c4ccccc4CNCc4ccccc4)s3)c2cc1OC]

A mixture of N-[1-(5-bromothiophen-2-yl)ethyl]-6,7-dimethoxy-2-methylquinazolin-4-amine (prepared as described in example 209; 100 mg, 0.24 mmol), 2-(N-benzylaminomethyl)phenylboronic acid, pinacol ester (commercially available; 80.8 mg, 0.24 mmol), potassium carbonate (135.4 mg, 0.98 mmol) and tetrakis(triphenylphosphin) palladium(0) (28.3 mg, 0.025 mmol) in dioxane (2.5 mL) and water (0.5 mL) was stirred at 110° C. overnight. The reaction mixture was evaporated and the crude product was purified via HPLC to yield 48% of the title compound (63 mg). ¹H-NMR (400 MHz, DMSO-d₆): δ [ppm]=8.14 (d, 1H), 7.65 (s, 1H), 7.54 (d, 1H), 7.34-7.22 (m, 7H), 7.22-7.14 (m, 1H), 7.11 (d, 1H), 7.06-7.03 (m, 2H), 5.96 (t, 1H), 3.86 (d, 6H), 3.73-3.61 (m, 4H), 2.54-2.52 (m, 1H), 2.44-2.40 (m, 3H), 1.71 (d, 3H). LC-MS (method 7): m/z: [M+H]⁺=525.2, Rₜ=0.66 min.

### Example 366

### N-[1-(5-{2-[(butylamino)methyl]phenyl}thiophen-2-yl)ethyl]-6,7-dimethoxy-2-methylquinazolin-4-amine

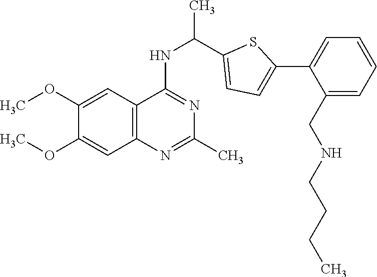 [SMILES: CCCCNCc1ccccc1-c1ccc(C(C)Nc2nc(C)nc3cc(OC)c(OC)cc23)s1]

A mixture of N-[1-(5-bromothiophen-2-yl)ethyl]-6,7-dimethoxy-2-methylquinazolin-4-amine (prepared as described in example 209; 100 mg, 0.24 mmol), 2-(N-butylaminomethyl)phenylboronic acid, pinacol ester (commercially available; 73.8 mg, 0.24 mmol), potassium carbonate (135.4 mg, 0.98 mmol) and tetrakis(triphenylphosphin) palladium(0) (28.3 mg, 0.025 mmol) in dioxane (2.5 mL) and water (0.5 mL) was stirred at 110° C. overnight. The reaction mixture was evaporated and the crude product was purified via HPLC to yield 46% of the title compound (56 mg). ¹H-NMR (400 MHz, DMSO-d₆): δ [ppm]=8.14 (d, 1H), 7.65 (s, 1H), 7.49 (d, 1H), 7.33-7.22 (m, 3H), 7.16 (d, 1H), 7.07 (d, 1H), 7.05 (s, 1H), 5.96 (t, 1H), 3.68 (s, 2H), 3.33 (s, 3H), 2.43 (s, 5H), 1.71 (d, 3H), 1.36-1.19 (m, 4H), 0.82 0.77 (m, 3H). LC-MS (method 7): m/z: [M+H]⁺=491.3, Rₜ=0.63 min.

### Example 367

### N-[1-(5-{2-[(ethylamino)methyl]phenyl}thiophen-2-yl)ethyl]-6,7-dimethoxy-2-methylquinazolin-4-amine

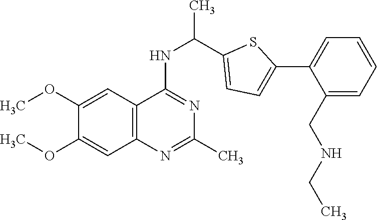 [SMILES: CCNCc1ccccc1-c1ccc(C(C)Nc2nc(C)nc3cc(OC)c(OC)cc23)s1]

A mixture of N-[1-(5-bromothiophen-2-yl)ethyl]-6,7-dimethoxy-2-methylquinazolin-4-amine (prepared as described in example 209; 100 mg, 0.24 mmol), 2-(ethylaminomethyl)phenylboronic acid, pinacol ester (commercially available; 66.6 mg, 0.24 mmol), potassium carbonate (135.4 mg, 0.98 mmol) and tetrakis(triphenylphosphin) palladium(0) (28.3 mg, 0.025 mmol) in dioxane (2.5 mL) and water (0.5 mL) was stirred at 110° C. overnight. The reaction mixture was evaporated and the crude product was purified via HPLC to yield 31% of the title compound (36 mg). ¹H-NMR (400 MHz, DMSO-d₆): δ [ppm]=8.14 (d, 1H), 7.65 (s, 1H), 7.49 (d, 1H), 7.33-7.22 (m, 3H), 7.16 (d, 1H), 7.05 (s, 1H), 5.96 (t, 1H), 3.68 (s, 2H), 3.33 (s, 3H), 2.43 (s, 3H), 1.71 (d, 3H), 0.95 (t, 3H). LC-MS (method 7): m/z: [M+H]⁺=463.2, Rₜ=0.59 min.

### Example 368

### 6,7-dimethoxy-2-methyl-N-[1-{5-[2-(1H-tetrazol-5-yl)phenyl]thiophen-2-yl}ethyl]-quinazolin-4-amine

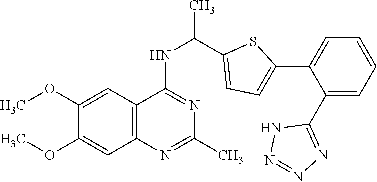 [SMILES: COc1cc2nc(C)nc(NC(C)c3ccc(-c4ccccc4-c4nnn[nH]4)s3)c2cc1OC]

A mixture of N-[1-(5-bromothiophen-2-yl)ethyl]-6,7-dimethoxy-2-methylquinazolin-4-amine (prepared as described in example 209; 100 mg, 0.24 mmol), 2-(tetrazole-5-yl)-phenylboronic acid (commercially available; 49 mg, 0.24 mmol), potassium carbonate (135.4 mg, 0.98 mmol) and tetrakis(triphenylphosphin) palladium(0) (28.3 mg, 0.025 mmol) in dioxane (2.5 mL) and water (0.5 mL) was stirred at 110° C. overnight. The reaction mixture was evaporated and the crude product was purified via HPLC to yield 28% of the title compound (33 mg). ¹H-NMR (400 MHz, DMSO-d₆): δ [ppm]=8.45 (br d, 1H), 7.66 (s, 1H), 7.61-7.53 (m, 2H), 7.51-7.43 (m, 2H), 7.04 (s, 1H), 6.94 (dd, 1H), 6.62 (d, 1H), 5.86 (t, 1H), 3.88 (d, 6H), 2.48-2.45 (m, 3H), 1.64 (d, 3H). LC-MS (method 7): m/z: [M+H]⁺=474.2, Rₜ=0.76 min.

### Example 369

### 6,7-dimethoxy-N-{1-[5-(2-{[(2-methoxyethyl)amino]methyl}phenyl)thiophen-2-yl]ethyl}-2-methylquinazolin-4-amine

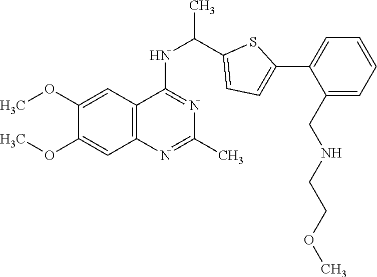 [SMILES: COCCNCc1ccccc1-c1ccc(C(C)Nc2nc(C)nc3cc(OC)c(OC)cc23)s1]

A mixture of N-[1-(5-bromothiophen-2-yl)ethyl]-6,7-dimethoxy-2-methylquinazolin-4-amine (prepared as described in example 209; 100 mg, 0.24 mmol), 2-(2-methoxyethyl)aminomethylpphenylboronic acid, pinacol ester (commercially available; 74.3 mg, 0.24 mmol), potassium carbonate (135.4 mg, 0.98 mmol) and tetrakis(triphenylphosphin)palladium(0) (28.3 mg, 0.025 mmol) in dioxane (2.5 mL) and water (0.5 mL) was stirred at 110° C. overnight. The reaction mixture was evaporated and the crude product was purified via HPLC to yield 50% of the title compound (61 mg). ¹H-NMR (400 MHz, DMSO-d₆): δ [ppm]=8.15 (d, 1H), 7.65 (s, 1H), 7.48 (d, 1H), 7.33-7.23 (m, 3H), 7.15 (d, 1H), 7.08 (d, 1H), 7.06-7.06 (m, 1H), 7.05 (s, 1H), 5.96 (t, 1H), 3.71 (s, 2H), 3.33 (s, 3H), 3.33-3.29 (m, 2H), 3.16 (s, 3H), 2.60 (t, 2H), 2.43 (s, 3H), 1.72 (d, 3H). LC-MS (method 7): m/z: [M+H]⁺=493.2, Rₜ=0.58 min.

### Example 370

### N-[1-(5-{2-[(cyclopropylamino)methyl]phenyl}thiophen-2-yl)ethyl]-6,7-dimethoxy-2-methylquinazolin-4-amine

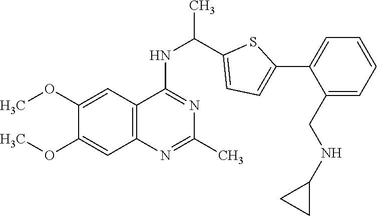 [SMILES: COc1cc2nc(C)nc(NC(C)c3ccc(-c4ccccc4CNC4CC4)s3)c2cc1OC]

A mixture of N-[1-(5-bromothiophen-2-yl)ethyl]-6,7-dimethoxy-2-methylquinazolin-4-amine (prepared as described in example 209; 100 mg, 0.24 mmol), 2-(N-cyclopropylaminoethyl)penylboronic acid, pinacol ester (commercially available; 47.7 mg, 0.24 mmol), potassium carbonate (135.4 mg, 0.98 mmol) and tetrakis(triphenylphosphin) palladium(0) (28.3 mg, 0.025 mmol) in dioxane (2.5 mL) and water (0.5 mL) was stirred at 110° C. overnight. The reaction mixture was evaporated and the crude product was purified via HPLC to yield 3% of the title compound (3.6 mg). LC-MS (method 7): m/z: [M+H]⁺=475.2, Rₜ=0.58 min.

### Example 371

### methyl 4-{[(1R)-1-(3-chlorophenyl)ethyl]amino}-2-methylquinazoline-6-carboxylate

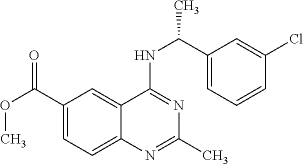 [SMILES: COC(=O)c1ccc2nc(C)nc(N[C@H](C)c3cccc(Cl)c3)c2c1]

In an autoclave a solution of 6-bromo-N-[(1R)-1-(3-chlorophenyl)ethyl]-2-methylquinazolin-4-amine (prepared as described in example 363; 9.5 g, 25.22 mmol) was dissolved in a 10:1 methanol:THF mixture (440 mL) and 1,1-Bis-(diphenylphosphino)-ferrocen-palladium(II)dichloride (1 g, 1.26 mmol), triethylamine (7 mL, 50.44 mmol) were added. At ambient temperature the reaction mixture was flushed with CO gas (3×) and then carbonylated at 14.77 bar CO at 80° C. overnight. The reaction mixture was evaporated and the crude product was purified via Isolera flash chromatography (Silica column; eluent: gradient hexanes 100%→ethylacetate 100%) to yield the title compound (8.9 g, 95%). ¹H-NMR (400 MHz, DMSO-d₆): δ [ppm]=9.10 (d, 1H), 8.90 (d, 1H), 8.18 (dd, 1H), 7.64 (d, 1H), 7.53 (t, 1H), 7.44-7.40 (m, 1H), 7.36 (t, 1H), 7.31-7.26 (m, 1H), 5.63 (t, 1H), 3.93 (s, 3H), 2.42 (s, 3H), 1.60 (d, 3H). LC-MS (method 7): m/z: [M+H]⁺=356.1, Rₜ=0.82 min.

### Example 372

### 4-{[(1R)-1-(3-chlorophenyl)ethyl]amino}-2-methylquinazoline-6-carboxylic acid

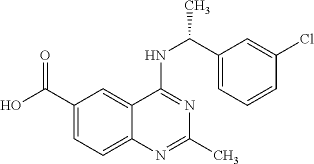 [SMILES: Cc1nc(N[C@H](C)c2cccc(Cl)c2)c2cc(C(=O)O)ccc2n1]

To a solution of methyl 4-{[(1R)-1-(3-chlorophenyl)ethyl]amino}-2-methylquinazoline-6-carboxylate (prepared as described in example 371; 1.5 g, 4.22 mmol) in ethanol (40 mL) was added NaOH beads (674 mg, 16.9 mmol) and the reaction was stirred at ambient temperature for two hours. Water was added (100 mL) and the pH value was adjusted to pH3 by the addition of 1.0 N aqueous HCl. The precipitate was collected by filtration and the product was dried under reduced pressure to yield the title compound in 95% yield (1.38 g). ¹H-NMR (400 MHz, DMSO-d₆): δ [ppm]=13.33-13.06 (m, 1H), 9.09 (d, 1H), 8.87 (d, 1H), 8.17 (dd, 1H), 7.62 (d, 1H), 7.53 (t, 1H), 7.44-7.41 (m, 1H), 7.35 (t, 1H), 7.30-7.26 (m, 1H), 5.62 (quin, 1H), 2.45-2.40 (m, 3H), 1.59 (d, 3H). LC-MS (method 7): m/z: [M+H]⁺=342.1, Rₜ=0.75 min.

### Example 373

### (4-{[(1R)-1-(3-chlorophenyl)ethyl]amino}-2-methylquinazolin-6-yl)methanol

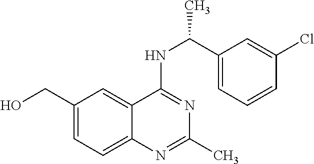 [SMILES: Cc1nc(N[C@H](C)c2cccc(Cl)c2)c2cc(CO)ccc2n1]

To a solution of 4-{[(1R)-1-(3-chlorophenyl)ethyl]amino}-2-methylquinazoline-6-carboxylic acid (prepared as described in example 372; 600 mg, 1.75 mmol) in THF (30 mL) was added LiAlH₄ solution (2.0 M in THF, 3.5 mL) at ambient temperature. The reaction was allowed to stir at ambient temperature for 30 minutes, then methanol (5 mL) was added and the reaction mixture was poured carefully into saturated aqueous ammoniumchloride solution. The reaction mixture was extracted with butanol (3×10 mL), the combined organic layers were evaporated and the crude product was purified via HPLC chromatography to yield the title compound (74 mg, 13% yield). ¹H-NM R (400 MHz, DMSO-d₆): δ [ppm]=9.35-8.77 (m, 1H), 8.41 (s, 1H), 8.13 (s, 1H), 7.82-7.71 (m, 1H), 7.61 (d, 1H), 7.53 (s, 1H), 7.42 (s, 1H), 7.36 (s, 1H), 7.31 (s, 1H), 5.82 5.55 (m, 1H), 5.55-5.29 (m, 1H), 4.66 (br d, 2H), 1.60 (d, 3H). LC-MS (method 7): m/z: [M+H]⁺=328.1, Rₜ=0.72 min.

### Example 374

### [2-(5-{1-[(6,7-dimethoxy-2-methylquinazolin-4-yl)amino]ethyl}thiophen-2-yl)-phenyl](phenyl)methanol

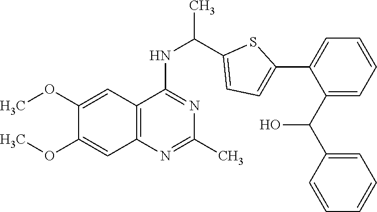 [SMILES: COc1cc2nc(C)nc(NC(C)c3ccc(-c4ccccc4C(O)c4ccccc4)s3)c2cc1OC]

To a solution of 2-(5-{1-[(6,7-dimethoxy-2-methylquinazolin-4-yl)amino]ethyl}thiophen-2-yl)benzaldehyde (described in example 179a; 50.0 mg, 115 μmol) in anhydrous THF (1.0 mL) was added bromo(phenyl)magnesium (250 μL, 1.0 M, 250 μmol) dropwise and the reaction mixture stirred during 30 minutes at room temperature. The reaction was quenched with H₂O, the mixture extracted with DCM and the solvent removed in vacuo. Purification by column chromatography (silica gel, MeOH/EtOAc 0-20% then MeOH) gave the title compound as a white solid (16.5 mg, 27%). ¹H-NMR (500 MHz, DMSO-d₆): δ [ppm]=8.14 (dd, 1H), 7.65 (d, 1H), 7.44 (d, 1H), 7.36-7.32 (m, 1H), 7.30-7.24 (m, 2H), 7.22-7.17 (m, 2H), 7.16-7.12 (m, 1H), 7.12-7.09 (m, 2H), 7.07 (d, 1H), 7.05 (s, 1H), 6.91 (d, 1H), 5.97 (t, 1H), 5.95-5.90 (m, 1H), 5.86 (dd, 1H), 3.87 (s, 6H), 2.42 (d, 3H), 1.71 (d, 3H). LC-MS (method 7): m/z: [M+H]⁺=512, Rt=0.98 min.

### Example 375

### 1-[2-(5-{1-[(6,7-dimethoxy-2-methylquinazolin-4-yl)amino]ethyl}thiophen-2-yl)phenyl]-3-phenylpropan-1-ol

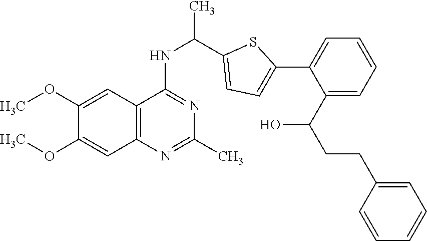 [SMILES: COc1cc2nc(C)nc(NC(C)c3ccc(-c4ccccc4C(O)CCc4ccccc4)s3)c2cc1OC]

To a solution of 2-(5-{1-[(6,7-dimethoxy-2-methylquinazolin-4-yl)amino]ethyl}thiophen-2-yl)benzaldehyde (described in example 179a; 50.0 mg, 115 μmol) in anhydrous THE (1.0 mL) was added chloro(2-phenylethyl)magnesium (250 μL, 1.0 M, 250 μmol) dropwise and the reaction mixture stirred during 30 minutes at room temperature. The reaction was quenched with H₂O, the mixture extracted with DCM and the solvent removed in vacuo. Purification by preparative HPLC (basic conditions) gave the title compound as a white solid (32.7 mg, 51%). ¹H-NMR (400 MHz, DMSO-d₆): δ [ppm]=8.14 (dd, 1H), 7.65 (s, 1H), 7.62 (d, 1H), 7.36 (dt, 1H), 7.24 (d, 2H), 7.20-7.07 (m, 3H), 7.06-7.00 (m, 3H), 6.99-6.94 (m, 1H), 6.76 (t, 1H), 5.95 (br t, 1H), 5.20 (t, 1H), 4.88-4.76 (m, 1H), 3.88-3.83 (m, 6H), 2.70-2.61 (m, 1H), 2.43 (s, 3H), 1.80 (br d, 2H), 1.71 (dd, 3H). LC-MS (method 7): m/z: [M+H]⁺=540, Rt=1.06 min.

### Example 376

### 1-[2-(5-{1-[(6,7-dimethoxy-2-methylquinazolin-4-yl)amino]ethyl}thiophen-2-yl)phenyl]-2-phenylethanol

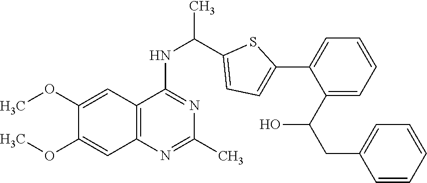 [SMILES: COc1cc2nc(C)nc(NC(C)c3ccc(-c4ccccc4C(O)Cc4ccccc4)s3)c2cc1OC]

To a solution of 2-(5-{1-[(6,7-dimethoxy-2-methylquinazolin-4-yl)amino]ethyl}thiophen-2-yl)benzaldehyde (described in example 179a; 50.0 mg, 115 μmol) in anhydrous THF (1.0 mL) was added benzyl(chloro)magnesium (130 μL, 2.0 M, 250 μmol) dropwise and the reaction mixture stirred during 30 minutes at room temperature. The reaction was quenched with H₂O, the mixture extracted with DCM and the solvent removed in vacuo. Purification by preparative HPLC (basic conditions) gave the title compound as a light yellow solid (19.5 mg, 32%). ¹H-NMR (400 MHz, DMSO-d₆): δ [ppm]=8.15 (d, 1H), 7.70-7.64 (m, 2H), 7.43-7.37 (m, 1H), 7.29 7.20 (m, 2H), 7.10-7.04 (m, 5H), 7.00-6.93 (m, 2H), 6.82 (dd, 1H), 6.02-5.91 (m, 1H), 5.17 (dd, 1H), 5.00 (dt, 1H), 3.89-3.85 (m, 6H), 2.84 (dt, 1H), 2.73 (dd, 1H), 2.42 (d, 3H), 1.73 (d, 3H). LC-MS (method 7): m/z: [M+H]⁺=526, Rt=1.02 min.

### Example 377

### 1-[2-(5-{1-[(6,7-dimethoxy-2-methylquinazolin-4-yl)amino]ethyl}thiophen-2-yl)phenyl]pentan-1-ol

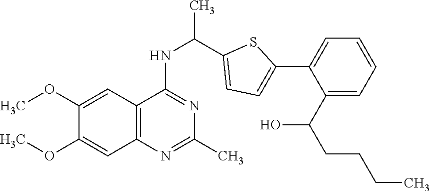 [SMILES: CCCCC(O)c1ccccc1-c1ccc(C(C)Nc2nc(C)nc3cc(OC)c(OC)cc23)s1]

To a solution of 2-(5-{1-[(6,7-dimethoxy-2-methylquinazolin-4-yl)amino]ethyl}thiophen-2-yl)benzaldehyde (described in example 179a; 50.0 mg, 115 μmol) in anhydrous THF (1.0 mL) was added butyl(chloro)magnesium (130 μL, 2.0 M, 250 μmol) dropwise and the reaction mixture stirred during 30 minutes at room temperature. The reaction was quenched with H2O, the mixture extracted with DCM and the solvent removed in vacuo. Purification by preparative HPLC (basic conditions) gave the title compound as a white solid (22.0 mg, 39%). ¹H-NMR (400 MHz, DMSO-d₆): δ [ppm]=8.13 (br d, 1H), 7.65 (s, 1H), 7.57 (d, 1H), 7.35 (dt, 1H), 7.24 7.21 (m, 2H), 7.07 (dd, 1H), 7.04 (s, 1H), 6.90 (d, 1H), 5.99-5.90 (m, 1H), 5.03 (d, 1H), 4.83 4.75 (m, 1H), 3.87 (s, 3H), 3.86 (s, 3H), 2.42 (s, 3H), 1.72 (d, 3H), 1.54-1.45 (m, 2H), 1.33-1.21 (m, 1H), 1.18-1.05 (m, 3H), 0.76-0.65 (m, 3H). LC-MS (method 7): m/z: [M+H]⁺=492, Rt=1.03 min.

### Example 378

### 1-[2-(5-{1-[(6,7-dimethoxy-2-methylquinazolin-4-yl)amino]ethyl}thiophen-2-yl)phenyl]prop-2-yn-1-ol

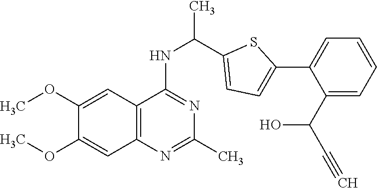 [SMILES: C#CC(O)c1ccccc1-c1ccc(C(C)Nc2nc(C)nc3cc(OC)c(OC)cc23)s1]

To a solution of 2-(5-{1-[(6,7-dimethoxy-2-methylquinazolin-4-yl)amino]ethyl}thiophen-2-yl)benzaldehyde (described in example 179a; 50.0 mg, 115 μmol) in anhydrous THF (1.0 mL) was added bromo(ethynyl)magnesium (510 μL, 0.50 M, 250 μmol) dropwise and the reaction mixture stirred during 30 minutes at room temperature. The reaction was quenched with H2O, the mixture extracted with DCM and the solvent removed in vacuo. Purification by preparative HPLC (basic conditions) gave the title compound as a white solid (23.6 mg, 45%). ¹H-NMR (400 MHz, DMSO-d₆): δ [ppm]=8.16 (d, 1H), 7.76 (d, 1H), 7.65 (s, 1H), 7.44-7.38 (m, 1H), 7.36-7.29 (m, 2H), 7.12 (d, 1H), 7.10 (dd, 1H), 7.05 (s, 1H), 6.08 (br s, 1H), 5.96 (quin, 1H), 5.41 (br s, 1H), 3.87 (s, 6H), 3.51 (t, 1H), 2.44 (s, 3H), 1.72 (d, 3H). LC-MS (method 7): m/z: [M+H]⁺=460, Rt=0.88 min.

### Example 379

### 1-[2-(5-{1-[(6,7-dimethoxy-2-methylquinazolin-4-yl)amino]ethyl}thiophen-2-yl)phenyl]-2-methylpropan-1-ol

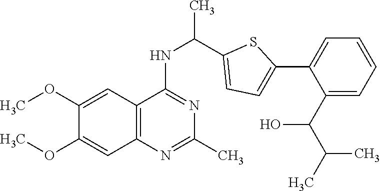 [SMILES: COc1cc2nc(C)nc(NC(C)c3ccc(-c4ccccc4C(O)C(C)C)s3)c2cc1OC]

To a solution of 2-(5-{1-[(6,7-dimethoxy-2-methylquinazolin-4-yl)amino]ethyl}thiophen-2-yl)benzaldehyde (described in example 179a; 50.0 mg, 115 μmol) in anhydrous THF (1.0 mL) was added lithium chloride-chloro(propan-2-yl)magnesium (1:1:1) (85 μL, 3.0 M, 250 μmol) dropwise and the reaction mixture stirred during 30 minutes at room temperature. The reaction was quenched with H₂O, the mixture extracted with DCM and the solvent removed in vacuo. Purification by preparative HPLC (basic conditions) gave the title compound as a white solid (8.10 mg, 14%). ¹H-NMR (400 MHz, DMSO-d₆): δ [ppm]=8.15 (br d, 1H), 7.65 (s, 1H), 7.53 (d, 1H), 7.35 (dt, 1H), 7.23 (dd, 2H), 7.06 (d, 1H), 7.05 (s, 1H), 6.93 (d, 1H), 5.95 (br quin, 1H), 5.02 (d, 1H), 4.58 (dd, 1H), 3.87 (s, 6H), 2.42 (d, 3H), 1.79-1.74 (m, 1H), 1.72 (d, 3H), 0.82 (dd, 3H), 0.61 (d, 3H). LC-MS (method 7): m/z: [M+H]⁺=478, Rt=0.99 min.

### Example 380

### 1-[2-(5-{1-[(6,7-dimethoxy-2-methylquinazolin-4-yl)amino]ethyl}thiophen-2-yl)phenyl]-2,2,2-trifluoroethanol

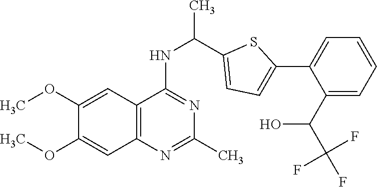 [SMILES: COc1cc2nc(C)nc(NC(C)c3ccc(-c4ccccc4C(O)C(F)(F)F)s3)c2cc1OC]

To a solution of 2-(5-{1-[(6,7-dimethoxy-2-methylquinazolin-4-yl)amino]ethyl}thiophen-2-yl)benzaldehyde (described in example 179a; 50.0 mg, 115 μmol) and trimethyl(trifluoromethyl)silane (82 μL, 575 μmol) in THE (1.0 mL) was added TBAF (200 μL, 1.0 M, 200 μmol) and the mixture stirred at room temperature during 1 hour. The reaction was quenched with HCl (1.0 M, 3.0 mL) and stirred at room temperature during 30 minutes, then extracted with DCM and the solvent removed in vacuo. Purification by preparative HPLC (basic conditions) gave the title compound as a yellow solid (34.2 mg, 59%). ¹H-NMR (400 MHz, DMSO-d₆): δ [ppm]=8.17 (dd, 1H), 7.68 (d, 1H), 7.64 (s, 1H), 7.47 (td, 1H), 7.42 (td, 1H), 7.37 7.33 (m, 1H), 7.12 (d, 1H), 7.05 (s, 1H), 6.91 (dd, 1H), 5.95 (quin, 1H), 5.30 (qd, 1H), 3.87 (s, 6H), 3.20-3.11 (m, 1H), 2.43 (d, 3H), 1.73 (dd, 3H). LC-MS (method 7): m/z: [M+H]⁺=504, Rt=0.97 min.

### Example 381

### N-{1-[5-(6,7-dihydro-5H-pyrrolo[1,2-a]imidazol-3-yl)-4-methylthiophen-2-yl]ethyl}-6,7-dimethoxy-2-methylquinazolin-4-amine

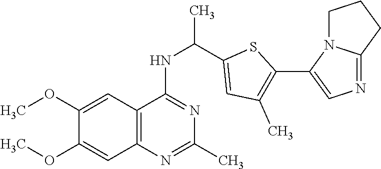 [SMILES: COc1cc2nc(C)nc(NC(C)c3cc(C)c(-c4cnc5n4CCC5)s3)c2cc1OC]

### Step a

### N-[1-(5-bromo-4-methylthiophen-2-yl)ethyl]-6,7-dimethoxy-2-methylquinazolin-4-amine hydrochloride (1:1)

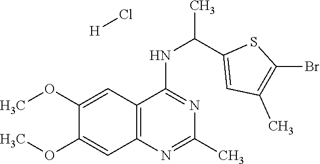 [SMILES: COc1cc2nc(C)nc(NC(C)c3cc(C)c(Br)s3)c2cc1OC ; Cl]

A solution of 4-chloro-6,7-dimethoxy-2-methylquinazoline (commercially available; 789 mg, 3.30 mmol) and 1-(5-bromo-4-methyl-2-thienyl)ethanamine (described in procedure INT-31; 800 mg, 3.63 mmol) in dioxane (15 mL) was heated to 110° C. during 18 hours. MTBE (20 mL) was added at room temperature, the mixture stirred during 3 hours and the precipitate then filtered to give the title compound as an off-white solid (1.56 g, 93%). ¹H-NMR (400 MHz, DMSO-d₆): δ [ppm]=8.04 (d, 1H), 7.60 (s, 1H), 7.05 (s, 1H), 6.88 (s, 1H), 5.76 (quin, 1H), 3.87 (s, 6H), 2.42 (s, 3H), 2.09 (s, 3H), 1.64 (d, 3H). LC-MS (method 7): m/z: [M+H]⁺=422, Rₜ=0.97 min.

### Step b

### N-{1-[5-(6,7-dihydro-5H-pyrrolo[1,2-a]imidazol-3-yl)-4-methylthiophen-2-yl]ethyl}-6,7-dimethoxy-2-methylquinazolin-4-amine

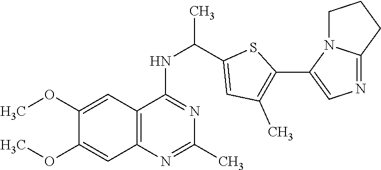 [SMILES: COc1cc2nc(C)nc(NC(C)c3cc(C)c(-c4cnc5n4CCC5)s3)c2cc1OC]

Under argon, N-[1-(5-bromo-4-methylthiophen-2-yl)ethyl]-6,7-dimethoxy-2-methylquinazolin-4-amine hydrochloride (1:1) (100 mg, 218 μmol), 6,7-dihydro-5H-pyrrolo[1,2-a]imidazol-3-ylboronic acid (33.1 mg, 218 μmol), K₂CO₃ (120 mg, 872 μmol) and Pd(PPh₃)₄ (25.2 mg, 21.8 μmol) in dioxane (2.0 mL) and H₂O (400 μL) were stirred at 110° C. overnight. H₂O was added, the mixture extracted with DCM and the solvent removed in vacuo. Purification by preparative HPLC (basic conditions) followed by column chromatography (silica gel, MeOH/EtOAc 0-20% then MeOH) gave the title compound as a white solid (23.8 mg, 24%). ¹H-NMR (400 MHz, DMSO-d₆): δ [ppm]=8.08 (d, 1H), 7.63 (s, 1H), 7.04 (s, 1H), 6.95 (s, 2H), 5.87 (quin, 1H), 3.93 (t, 2H), 3.86 (s, 6H), 2.75 (t, 2H), 2.43 (s, 3H), 2.18 (s, 3H), 1.67 (d, 3H). LC-MS (method 7): m/z: [M+H]⁺=450, Rt=0.54 min.

### Example 382

### N-[2-(5-{1-[(6,7-dimethoxy-2-methylquinazolin-4-yl)amino]ethyl}thiophen-2-yl)-5-fluorobenzyl]-2-hydroxyacetamide

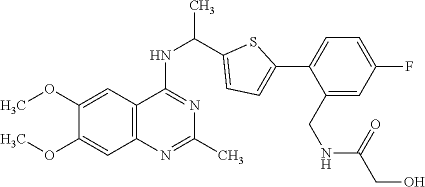 [SMILES: COc1cc2nc(C)nc(NC(C)c3ccc(-c4ccc(F)cc4CNC(=O)CO)s3)c2cc1OC]

A solution of N-[1-{5-[2-(aminomethyl)-4-fluorophenyl]thiophen-2-yl}ethyl]-6,7-dimethoxy-2-methylquinazolin-4-amine (described in example 234; 100 mg, 221 μmol), hydroxyacetic acid (16.8 mg, 221 μmol), PyBOP (230 mg, 442 μmol) and N,N-diisopropylethylamine (190 μL, 1.1 mmol) in DMF (1.0 mL) was stirred at room temperature overnight. Purification by preparative HPLC (basic conditions) gave the title compound as a white solid (61.7 mg, 54%). ¹H-NMR (400 MHz, DMSO-d₆): δ [ppm]=8.35 (t, 1H), 8.16 (d, 1H), 7.64 (s, 1H), 7.35 (dd, 1H), 7.13 7.07 (m, 3H), 7.06-7.03 (m, 2H), 5.96 (quin, 1H), 5.56 (t, 1H), 4.38 (d, 2H), 3.90-3.85 (m, 8H), 2.43 (s, 3H), 1.72 (d, 3H). LC-MS (method 7): m/z: [M+H]⁺=511, Rt=0.80 min.

### Example 383

### N-[2-(5-{1-[(6,7-dimethoxy-2-methylquinazolin-4-yl)amino]ethyl}thiophen-2-yl)-5-fluorobenzyl]-2-methoxyacetamide

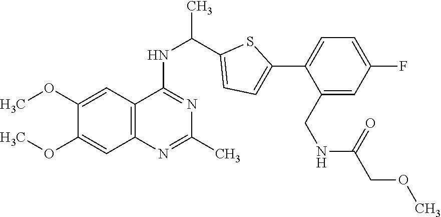

A solution of N-[1-{5-[2-(aminomethyl)-4-fluorophenyl]thiophen-2-yl}ethyl]-6,7-dimethoxy-2-methylquinazolin-4-amine (described in example 234; 50.0 mg, 110 μmol), methoxyacetic acid (8.5 μL, 110 μmol), PyBOP (115 mg, 221 μmol) and N,N-diisopropylethylamine (95 μL, 550 μmol) in DMF (1.0 mL) was stirred at room temperature overnight. Purification by preparative HPLC (basic conditions) gave the title compound as a pale brown solid (30.0 mg, 52%). ¹H-NMR (400 MHz, DMSO-d₆): δ [ppm]=8.38 (t, 1H), 8.16 (d, 1H), 7.64 (s, 1H), 7.35 (dd, 1H), 7.14-7.06 (m, 3H), 7.06-7.03 (m, 2H), 5.96 (quin, 1H), 4.37 (d, 2H), 3.87 (s, 8H), 3.31 (s, 3H), 2.43 (s, 3H), 1.72 (d, 3H). LC-MS (method 7): m/z: [M+H]⁺=525, Rt=0.88 min.

### Example 384

### N-(1-(5-(4-bromo-2-((dimethylamino)methyl)phenyl)thiophen-2-yl)ethyl)-6,7-dimethoxy-2-methylquinazolin-4-amine

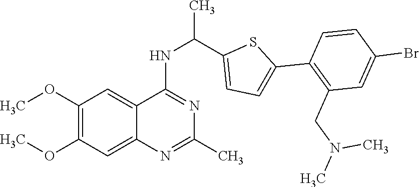 [SMILES: COc1cc2nc(C)nc(NC(C)c3ccc(-c4ccc(Br)cc4CN(C)C)s3)c2cc1OC]

### Step a

### 4-bromo-2-(bromomethyl)-1-iodobenzene

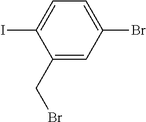

4-Bromo-1-iodo-2-methylbenzene, 5.00 g (16.8 mmol), and N-bromosuccinimide, 3.06 g (20.2 mmol), were dissolved into 50 mL of 1,2-dichloroethane. 2,2′-Azobisisobutyronitrile, 0.28 g (1.7 mmol), was added to the above solution at 85° C. and the resulting mixture was stirred at this temperature for 3 hours under nitrogen atmosphere. The solvent was removed in vacuo and the residue was purified by silica gel column chromatography to give 3.01 g (48%) of the product as a white solid. ¹H-NMR (400 MHz, CDCl₃): δ [ppm]=4.54 (s, 2H), 7.13-7.16 (m, 1H), 7.63 (d, 1H), 7.73 (d, 1H).

### Step b

### (5-bromo-2-iodophenyl)-N,N-dimethylmethanamine

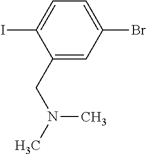

4-Bromo-2-(bromomethyl)-1-iodobenzene, 2.00 g (5.3 mmol), was dissolved into 10 mL of tetrahydrofuran and 5.3 mL of dimethylamine/THF solution (2.0 M). The resulting mixture was stirred at room temperature for 3 hours. The solvent was removed in vacuo and water was added. The mixture was extracted with dichloromethane and the combined organic layers were dried over anhydrous sodium sulfate. The solvent was removed in vacuo and the residue was purified by silica gel column chromatography to give 1.46 g (80%) of the product as a colourless oil. MS (ESIpos): m/z=340 [M+H]⁺; LC-MS [method 4, gradient starting with Water (0.05% TFA)-Acetonitrile, 5% B]: Rₜ=0.72 min.

### Step c

### 1-(5-(4-bromo-2-((dimethylamino)methyl)phenyl)thiophen-2-yl)ethanone

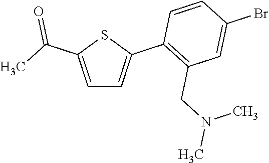 [SMILES: CC(=O)c1ccc(-c2ccc(Br)cc2CN(C)C)s1]

(5-Bromo-2-iodophenyl)-N,N-dimethylmethanamine, 1.41 g (4.1 mmol), 5-acetylthiophen-2-ylboronic acid, 1.06 g (6.2 mmol), sodium carbonate, 1.76 g (16.6 mmol) and 1,1′-bis(diphenylphosphino)ferrocenepalladium(II) chloride, 0.30 g (0.4 mmol), were added into 12 mL of 1,4-dioxane/H₂O (v:v=5:1). The resulting mixture was stirred at 100° C. for 48 hours under nitrogen atmosphere. After cooled to room temperature, water was added and the resulting mixture was extracted with ethyl acetate. The combined organic layers were dried over anhydrous sodium sulfate and the solvent was removed in vacuo. The residue was purified by silica gel column chromatography to give 0.67 g (30%) of the product as a yellow oil. MS (ESIpos): m/z=338 [M+H]⁺; LC-MS [method 4, gradient starting with Water (0.05% NH₄HCO₃)-Acetonitrile, 5% B]: Rₜ=2.17 min.

### Step d

### N-(1-(5-(4-bromo-2-((dimethylamino)methyl)phenyl)thiophen-2-yl)ethylidene)-2-methyl propane-2-sulfinamide

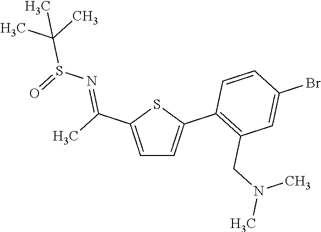 [SMILES: C/C(=N\S(=O)C(C)(C)C)c1ccc(-c2ccc(Br)cc2CN(C)C)s1]

1-(5-(4-Bromo-2-((dimethylamino)methyl)phenyl)thiophen-2-yl)ethanone, 620.0 mg (1.8 mmol), 2-methylpropane-2-sulfinamide, 444.3 mg (3.7 mmol), and titanium(IV) ethoxide, 836.2 mg (3.7 mmol), were dissolved into 20 mL of tetrahydrofuran and the resulting mixture was stirred at 70° C. for 36 hours under nitrogen atmosphere. After cooled to room temperature, 10% sodium chloride solution was added and the precipitated solid was removed by filtration. The filtrate was extracted with ethyl acetate and the combined organic layers were dried over anhydrous sodium sulfate. The solvent was removed in vacuo and the residue was purified by silica gel column chromatography to give 0.51 g (51%) of the product as a yellow oil. MS (ESIpos): m/z=441 [M+H]⁺; LC-MS [method 4, gradient starting with Water (0.05% TFA)-Acetonitrile, 5% B]: Rₜ=0.83 min.

### Step e

### N-(1-(5-(4-bromo-2-((dimethylamino)methyl)phenyl)thiophen-2-yl)ethyl)-2-methyl propane-2-sulfinamide

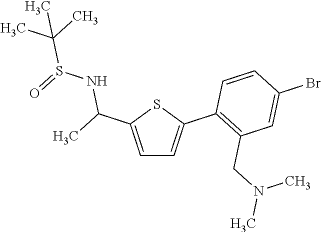 [SMILES: CC(NS(=O)C(C)(C)C)c1ccc(-c2ccc(Br)cc2CN(C)C)s1]

N-(1-(5-(4-Bromo-2-((dimethylamino)methyl)phenyl)thiophen-2-yl)ethylidene)-2-methylpropane-2-sulfinamide, 512.0 mg (1.2 mmol), was dissolved in 10 mL of tetrahydrofuran. Sodium borohydride, 87.8 mg (2.3 mmol), was added to the above solution portionwise. The resulting mixture was stirred at room temperature for 2 hours. Water was added and the resulting solution was extracted with ethyl acetate. The combined organic layers were dried over anhydrous sodium sulfate and concentrated in vacuo. The residue was purified by silica gel column chromatography to give 0.33 g (59%) of the product as a yellow oil. MS (ESIpos): m/z=443 [M+H]⁺; LC-MS [method 4, gradient starting with Water (0.05% TFA)-Acetonitrile, 5% B]: Rₜ=0.88 min.

### Step f

### 1-(5-(4-bromo-2-((dimethylamino)methyl)phenyl)thiophen-2-yl)ethanamine

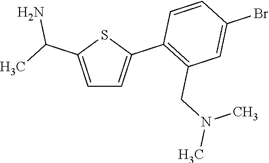 [SMILES: CC(N)c1ccc(-c2ccc(Br)cc2CN(C)C)s1]

N-(1-(5-(4-Bromo-2-((dimethylamino)methyl)phenyl)thiophen-2-yl)ethyl)-2-methylpropane-2-sulfinamide, 331.0 mg (0.7 mmol), was dissolved into 10 mL of tetrahydrofuran. Then 5 mL of conc. hydrochloric acid was added and the resulting mixture was stirred at room temperature for 24 hours. The solvent was removed in vacuo and saturated aqueous sodium carbonate was added to adjust the pH value to 8. The resulting mixture was extracted with dichloromethane and the combined organic layers were dried over anhydrous sodium sulfate. The solvent was removed in vacuo and the residue was purified by silica gel column chromatography to give 98.0 mg (42%) of the product as a brown oil. MS (ESIpos): m/z=339 [M+H]⁺; LC-MS [method 4, gradient starting with Water (0.05% NH₄HCO₃)-Acetonitrile, 5% B]: Rₜ=1.96 min.

### Step g

### N-(1-(5-(4-bromo-2-((dimethylamino)methyl)phenyl)thiophen-2-yl)ethyl)-6,7-dimethoxy-2-methylquinazolin-4-amine

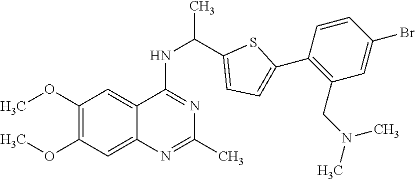 [SMILES: COc1cc2nc(C)nc(NC(C)c3ccc(-c4ccc(Br)cc4CN(C)C)s3)c2cc1OC]

1-(5-(4-Bromo-2-((dimethylamino)methyl)phenyl)thiophen-2-yl)ethanamine, 93.0 mg (0.3 mmol), and 4-chloro-6,7-dimethoxy-2-methylquinazoline, 98.1 mg (0.4 mmol), were dissolved into 2 mL of 2-propanol. The resulting mixture was stirred at 110° C. for 6 hours. After cooled to room temperature, the solvent was removed in vacuo and the residue was purified by preparative HPLC to give 58.4 mg (38%) of the product as a light yellow solid. MS (ESIpos): m/z=541 [M+H]⁺; LC-MS [method 4, gradient starting with Water (0.05% TFA)-Acetonitrile, 5% B]: Rₜ=1.11 min. ¹H-NMR (400 MHz, DMSO-d₆): δ [ppm]=1.72 (d, 3H), 2.11 (s, 6H), 2.43 (s, 3H), 3.38 (s, 2H), 3.87 (s, 6H), 5.94-5.97 (m, 1H), 7.05 (s, 1H), 7.09 (s, 1H), 7.15 (d, 1H), 7.30 (d, 1H), 7.46 (d, 1H), 7.63-7.64 (m, 2H), 8.16 (d, 1H).

### Example 385

### N-(1-(5-(2-((dimethylamino) methyl)-4-(trifluoromethyl) phenyl) thiophen-2-yl) ethyl)-6,7-dimethoxy-2-methylquinazolin-4-amine

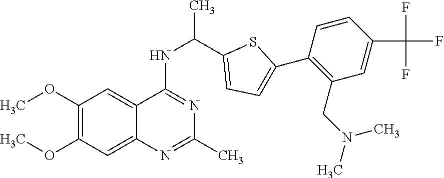 [SMILES: COc1cc2nc(C)nc(NC(C)c3ccc(-c4ccc(C(F)(F)F)cc4CN(C)C)s3)c2cc1OC]

### Step a

### 4,4,5,5-tetramethyl-2-(2-methyl-4-(trifluoromethyl)phenyl)-1,3,2-dioxaborolane

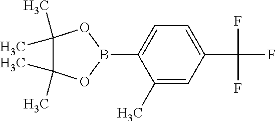 [SMILES: Cc1cc(C(F)(F)F)ccc1B1OC(C)(C)C(C)(C)O1]

1-Bromo-2-methyl-4-(trifluoromethyl)benzene, 200.0 mg (0.8 mmol), 4,4,4′,4′,5,5,5′,5′-octamethyl-2,2′-bi(1,3,2-dioxaborolane), 424.9 mg (1.7 mmol), potassium acetate, 328.5 mg (3.3 mmol), and 1,1′-bis(diphenylphosphino)ferrocenepalladium(II) chloride, 61.2 mg (0.08 mmol), were added into 6 mL of 1,4-dioxane. The resulting mixture was stirred at 100° C. for overnight. After cooled to room temperature, water was added and the resulting mixture was extracted with dichloromethane and the combined organic layers were dried over anhydrous sodium sulfate. The solvent was removed in vacuo and the residue was purified by silica gel column chromatography to give 198.0 mg (83%) of the product as a white solid. ¹H-NMR (400 MHz, CDCl₃): δ [ppm]=1.38 (s, 12H), 2.61 (s, 3H), 7.42-7.43 (m, 2H), 7.88 (d, 1H).

### Step b

### 2-(2-(bromomethyl)-4-(trifluoromethyl) phenyl)-4,4,5,5-tetramethyl-1,3,2-dioxaborolane

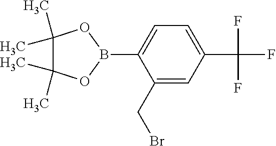 [SMILES: CC1(C)OB(c2ccc(C(F)(F)F)cc2CBr)OC1(C)C]

4,4,5,5-Tetramethyl-2-(2-methyl-4-(trifluoromethyl)phenyl)-1,3,2-dioxaborolane, 198.0 mg (0.7 mmol), and N-bromosuccinimide, 135.5 mg (0.8 mmol), were dissolved into 10 mL of 1,2-dichloroethane. The resulting mixture was heated to 80° C. and 2,2′-azobisisobutyronitrile, 11.4 mg (0.07 mmol), was added successively. The resulting mixture was stirred at 80° C. for 3 hours under nitrogen. The solvent was removed in vacuo and the residue was purified by silica gel column chromatography to give 165.0 mg (65%) of the product as a colorless oil. ¹H-NMR (400 MHz, CDCl₃): δ [ppm]=1.39 (s, 12H), 4.92 (s, 2H), 7.52 (d, 1H), 7.63 (s, 1H), 7.93 (d, 1H).

### Step c

### 2-((dimethylamino)methyl)-4-(trifluoromethyl)phenylboronic acid

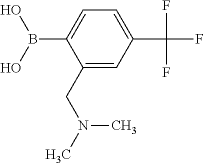 [SMILES: CN(C)Cc1cc(C(F)(F)F)ccc1B(O)O]

2-(2-(Bromomethyl)-4-(trifluoromethyl)phenyl)-4,4,5,5-tetramethyl-1,3,2-dioxaborolane, 165.0 mg (0.5 mmol), was added into 1.1 mL of dimethylamine/THF (2.0 M). The resulting mixture was stirred at room temperature for 3 hours. The solvent was removed in vacuo to give 121.0 mg (crude) of the product as a light yellow solid. MS (ESIpos): m/z=248 [M+H]⁺; LC-MS [Water (0.1% HCOOH)-Acetonitrile, 5% B)]: Rₜ=0.60 min.

### Step d

### N-(1-(5-(2-((dimethylamino)methyl)-4-(trifluoromethyl)phenyl)thiophen-2-yl)ethyl)-6,7-dimethoxy-2-methylquinazolin-4-amine

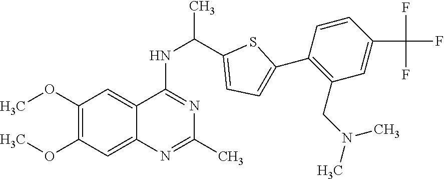 [SMILES: COc1cc2nc(C)nc(NC(C)c3ccc(-c4ccc(C(F)(F)F)cc4CN(C)C)s3)c2cc1OC]

2-((Dimethylamino)methyl)-4-(trifluoromethyl)phenylboronic acid, 100.0 mg (0.4 mmol), N-(1-(5-bromothiophen-2-yl)ethyl)-6,7-dimethoxy-2-methylquinazolin-4-amine, 165.3 mg (0.4 mmol), sodium carbonate, 171.6 mg (1.6 mmol) and tetrakis(triphenylphosphine)palladium(0), 46.8 mg (0.04 mmol), were added into 3 mL of 1,4-dioxane/H₂O (v:v=5:1). The resulting mixture was stirred at 100° C. for 6 hours under nitrogen atmosphere. After cooled to room temperature, water was added and the resulting mixture was extracted with ethyl acetate. The combined organic layers were dried over anhydrous sodium sulfate and the solvent was removed in vacuo. The residue was purified by preparative HPLC to give 34.8 mg (16%) of the product as a white solid. MS (ESIpos): m/z=531 [M+H]⁺; LC-MS [method 4, gradient starting with Water (0.05% TFA)-Acetonitrile, 5% B]: Rₜ=1.10 min. ¹H-NMR (400 MHz, DMSO-d₆): δ [ppm]=1.73 (d, 3H), 2.14 (s, 6H), 2.45 (s, 3H), 3.51 (s, 2H), 3.88 (s, 6H), 5.96-6.00 (m, 1H), 7.05 (s, 1H), 7.14 (d, 1H), 7.26 (d, 1H), 7.58-7.67 (m, 3H), 7.80 (s, 1H), 8.26 (d, 1H).

### Example 386

### 6,7-di methoxy-2-methyl-N-(1-{5-[2-methyl-4-(trifluoromethyl)phenyl]-2-thienyl}ethyl)quinazolin-4-amine

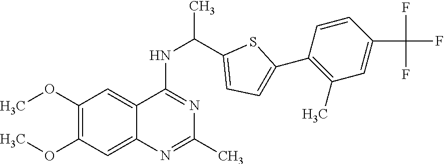 [SMILES: COc1cc2nc(C)nc(NC(C)c3ccc(-c4ccc(C(F)(F)F)cc4C)s3)c2cc1OC]

Under argon, N-[1-(5-bromothiophen-2-yl)ethyl]-6,7-dimethoxy-2-methylquinazolin-4-amine (50.0 mg, 122 μmol), 4,4,5,5-tetramethyl-2-[2-methyl-4-(trifluoromethyl)phenyl]-1,3,2-dioxaborolane (70.1 mg, 245 μmol), Na₂CO₃ (51.9 mg, 490 μmol) and 1,1′-bis(diphenylphosphino)ferrocenepalladium(II) chloride (8.96 mg, 12.2 μmol) in 1,4-dioxane (2.5 mL) and H₂O (500 μL) were stirred at 100° C. during 6 hours. H₂O was added, the mixture extracted with EtOAc, dried over anhydrous sodium sulphate and the solvent removed in vacuo. Purification by preparative HPLC (basic conditions) gave the title compound as an off-white solid (8.40 mg, 14%). ¹H-NMR (400 MHz, DMSO-d₆): δ [ppm]=8.17 (br d, 1H), 7.68 (br s, 1H), 7.66 (s, 1H), 7.56 (s, 2H), 7.21 (br d, 1H), 7.16 (br d, 1H), 7.06 (s, 1H), 5.98 (quin, 1H), 3.88 (s, 6H), 2.48 (s, 3H), 2.44 (s, 3H), 1.74 (br d, 3H). LC-MS (Method 4): m/z: [M+H]⁺=488, Rₜ=1.63 min.

### Example 387

### tert-butyl [4-chloro-2-(5-{1-[(6,7-dimethoxy-2-methylquinazolin-4-yl)amino]ethyl}-thiophen-2-yl)benzyl]carbamate

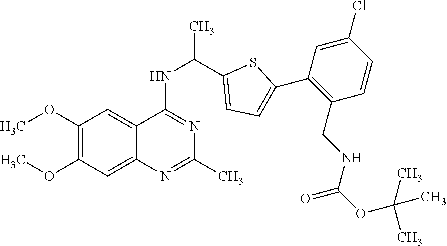 [SMILES: COc1cc2nc(C)nc(NC(C)c3ccc(-c4cc(Cl)ccc4CNC(=O)OC(C)(C)C)s3)c2cc1OC]

### Step a

### 1-(2-bromo-4-chlorophenyl)methanamine

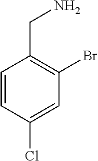

This compound was synthesized by the same method as described in example 408 (step a) to give 1.47 g (36%) of the product as an off-white solid. MS (ESIpos): m/z=220 [M+H]⁺. LC-MS [Method 4, Water (0.1% HCOOH)-Acetonitrile, 10% B]: Rₜ=0.62 min.

### Step b

### tert-butyl (2-bromo-4-chlorobenzyl)carbamate

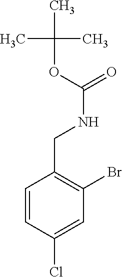 [SMILES: CC(C)(C)OC(=O)NCc1ccc(Cl)cc1Br]

This compound was synthesized by the same method as described in example 428 (step a) to give 1.71 g (74%) of the product as a light yellow solid. MS (ESIpos): m/z=320 [M+H]⁺. LC-MS [Method 4, Water (0.05% TFA)-Acetonitrile, 5% B]: Rₜ=1.21 min.

### Step c

### tert-butyl [4-chloro-2-(4,4,5,5-tetramethyl-1,3,2-dioxaborolan-2-yl)benzyl]carbamate

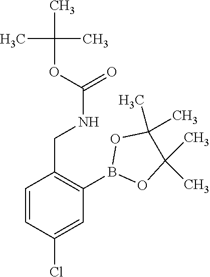 [SMILES: CC(C)(C)OC(=O)NCc1ccc(Cl)cc1B1OC(C)(C)C(C)(C)O1]

This compound was synthesized by the same method as described in example 428 (step b) to give 375 mg (36%) of the product as a light yellow solid. MS (ESIpos): m/z=368 [M+H]⁺. LC-MS [Method 4, Water (0.05% TFA)-Acetonitrile, 5% B]: Rₜ=1.42 min.

### Step d

### tert-butyl [4-chloro-2-(5-{1-[(6,7-dimethoxy-2-methylquinazolin-4-yl)amino]ethyl}-thiophen-2-yl)benzyl]carbamate

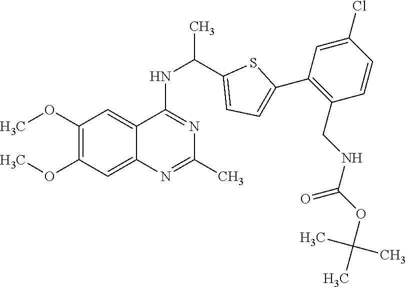 [SMILES: COc1cc2nc(C)nc(NC(C)c3ccc(-c4cc(Cl)ccc4CNC(=O)OC(C)(C)C)s3)c2cc1OC]

This compound was synthesized by the same method as described in example 429 to give 150 mg (63%) of the product as a light yellow solid. MS (ESIpos): m/z=569 [M+H]⁺. LC-MS [Method 4, Water (0.05% TFA)-Acetonitrile, 5% B]: Rₜ=1.56 min. ¹H-NMR (400 MHz, DMSO-d₆): δ [ppm]=1.37 (s, 9H), 1.72 (d, 3H), 2.44 (s, 3H), 3.87 (s, 6H), 4.20 (d, 2H), 5.95-5.99 (m, 1H), 7.05 (s, 1H), 7.10-7.12 (m, 2H), 7.32-7.44 (m, 4H), 7.65 (s, 1H), 8.17 (d, 1H).

### Example 388

### 4-(3-{(1R)-1-[(6,7-dimethoxy-2-methylquinazolin-4-yl)amino]ethyl}phenyl)pyridin-2-ol

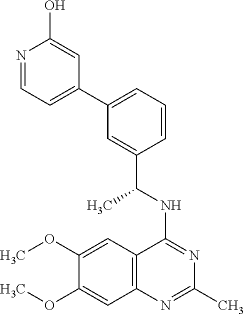

To a solution of 2-hydroxypyridine-4-boronic acid (46.8 mg, 0.32 mmol) and N-[(1R)-1-(3-bromophenyl)ethyl]-6,7-dimethoxy-2-methylquinazolin-4-amine (described in example 191, 100 mg, 0.25 mmol) in DMF (10 mL) were added potassium phosphate solution (0.5 M in water, 1.49 mL) and the second generation RuPhos precatalyst (chloro(2-dicyclohexylphosphino-2′,6′-diisopropoxy-1,1′-biphenyl)[2-(2′-amino-1,1′-biphenyl)]palladium(II), 38.6 mg, 0.05 mmol). The reaction mixture was stirred at 75° C. for two hours. More 2-hydroxypyridine-4-boronic acid (46.8 mg, 0.32 mmol), chloro(2-dicyclohexylphosphino-2′,6′-diisopropoxy-1,1′-biphenyl)[2-(2′-amino-1,1-biphenyl)]palladium(II) (38.6 mg, 0.05 mmol) and potassium phosphate solution (0.5 M in water, 1.49 mL) were added and the reaction was stirred at 75° C. overnight. The reaction mixture was filtered and the filtrate was evaporated. The title compound was obtained in 12% yield (12.4 mg) after HPLC chromatography. ¹H-NMR (400 MHz, DMSO-d₆): δ [ppm]=12.07-11.09 (m, 1H), 8.02 (d, 1H), 7.78 (s, 1H), 7.70 (s, 1H), 7.58-7.49 (m, 2H), 7.48-7.38 (m, 2H), 7.02 (s, 1H), 6.56 (d, 1H), 6.47 (dd, 1H), 5.69 (t, 1H), 3.91 (s, 3H), 3.86 (s, 3H), 2.39-2.28 (m, 3H), 1.63 (d, 3H). LC-MS (Method 8): m/z: [M+H]⁺=417, Rₜ=0.78 min.

### Example 389

### N-{1-[3-(benzyloxy)phenyl]ethyl}-6,7-dimethoxy-2-methylquinazolin-4-amine

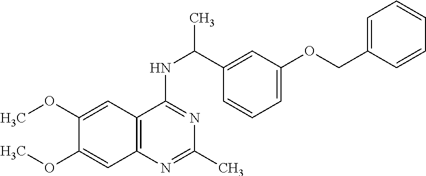 [SMILES: COc1cc2nc(C)nc(NC(C)c3cccc(OCc4ccccc4)c3)c2cc1OC]

### Step a

### 1-[3-(benzyloxy)phenyl]-N-hydroxyethanimine

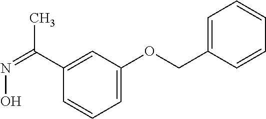 [SMILES: C/C(=N/O)c1cccc(OCc2ccccc2)c1]

A solution of 3-benzyloxyacetophenon (commercially available, 10.5 g, 46.4 mmol), hydroxylamine hydrochloride (16.1 g, 232 mmol), sodium acetate (464 mmol) in ethanol (250 mL) was stirred at 40° C. overnight. The solvent was distilled off under reduced pressure and the residue was extracted once with ethyl acetate/HCl (1.0 M in water). The aqueous phase was extracted with ethyl acetate (3×100 mL) and the combined organic layers were dried over sodium sulfate. The crude product was obtained in quantitative yield (11.5 g) and used without further purification in the next step. LC-MS (Method 7): m/z: [M+H]⁺=242.1, Rₜ=1.15 & 1.20 min. (E/Z isomers in ratio ˜2:1)

### Step b

### 1-[3-(benzyloxy)phenyl]ethanamine

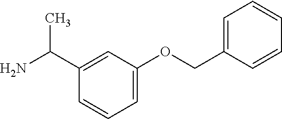 [SMILES: CC(N)c1cccc(OCc2ccccc2)c1]

To a solution of 1-[3-(benzyloxy)phenyl]-N-hydroxyethanimine (5.5 g, 22.79 mmol) in methanol (500 mL) was added Zn powder (44.7 g, 684 mmol) and ammonium chloride (42.7 g, 797.8 mmol) and the reaction was stirred at 60° C. overnight. The suspension was filtered and the residue was washed with methanol (3×100 mL). The filtrate was evaporated, water and basified by addition of ammonia solution (in water). The aqueous phase was extracted with ethyl acetate (3×100 mL) and the combined organic layers were dried over magnesium sulfate. After evaporation of the solvent the crude product was purified via Isolera flash chromatography (SNAP KP NH, 375 g column; eluent gradient hexanes (100%→hexanes/ethyl acetate (60:40)) to yield the title compound in 25% yield (1.35 g). LC-MS (Method 8): m/z: ¹H-NMR (400 MHz, DMSO-d₆): δ [ppm]=7.50-7.43 (m, 2H), 7.42-7.27 (m, 3H), 7.19 (t, 1H), 7.06-7.01 (m, 1H), 6.92 (d, 1H), 6.82 (ddd, 1H), 3.93 (q, 1H), 1.80 (br s, 2H), 1.21 (d, 3H). [M+H]⁺=228, Rₜ=0.76 min.

### Step c

### N-{1-[3-(benzyloxy)phenyl]ethyl}-6,7-dimethoxy-2-methylquinazolin-4-amine

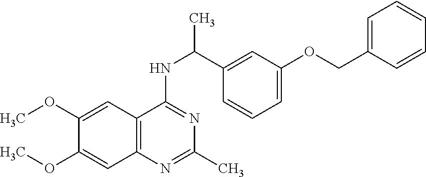 [SMILES: COc1cc2nc(C)nc(NC(C)c3cccc(OCc4ccccc4)c3)c2cc1OC]

A solution of 1-[3-(benzyloxy)phenyl]ethanamine (200 mg, 0.88 mmol), 4-chloro-6,7-dimethoxy-2-methylquinazoline (commercially available, 191 mg, 0.80 mmol), N,N-diisopropylethylamine (0.28 mL) in 1,4-dioxane (4.17 mL) was heated in a microwave oven for 7 hours at 120° C. The solvent was distilled off under reduced pressure and the residue purified by HPLC. The title compound was obtained in 45% yield (173 mg). ¹H-NMR (400 MHz, DMSO-d₆): δ [ppm]=8.13 (s, 2H), 7.73 (s, 1H), 7.44-7.20 (m, 6H), 7.09 (s, 1H), 7.03 (s, 2H), 6.93 6.84 (m, 1H), 5.83-5.41 (m, 1H), 5.07 (s, 2H), 3.90 (s, 3H), 3.87 (s, 3H), 2.36 (s, 3H), 1.57 (br d, 3H). LC-MS (Method 7): m/z: [M+H]⁺=430.2, Rₜ=0.99 min.

### Example 390

### N-[1-(5-{2-[(dimethylamino)methyl]phenyl}thiophen-2-yl)ethyl]-6,7-dimethoxy-2-methylquinazolin-4-amine (Enantiomer 1)

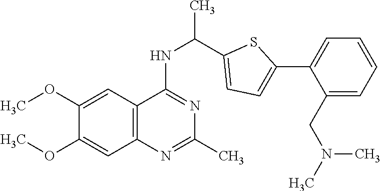 [SMILES: COc1cc2nc(C)nc(NC(C)c3ccc(-c4ccccc4CN(C)C)s3)c2cc1OC]

Enantiopure N-[1-(5-{2-[(dimethylamino)methyl]phenyl}-2-thienyl)ethyl]-6,7-dimethoxy-2-methylquinazolin-4-amine was obtained from racemic N-[1-(5-{2-[(dimethylamino)methyl]phenyl}-2-thienyl)ethyl]-6,7-dimethoxy-2-methylquinazolin-4-amine (described in example 262) by chiral HPLC purification (Method X7). ¹H-NMR (400 MHz, DMSO-d₆): δ [ppm]=8.15 (d, 1H), 7.65 (s, 1H), 7.44-7.40 (m, 1H), 7.38-7.35 (m, 1H), 7.33-7.26 (m, 2H), 7.18 (d, 1H), 7.08 (dd, 1H), 7.05 (s, 1H), 5.96 (quin, 1H), 3.87 (s, 6H), 3.36 (s, 2H), 2.43 (s, 3H), 2.10 (s, 6H), 1.72 (d, 3H). LC-MS (Method 7): m/z: [M+H]⁺=463, Rₜ=0.58 min. [α]_{D}=+163.8°+/−1.76°.

### Example 391

### N-[1-(5-{2-[(dimethylamino)methyl]phenyl}thiophen-2-yl)ethyl]-6,7-dimethoxy-2-methylquinazolin-4-amine (Enantiomer 2)

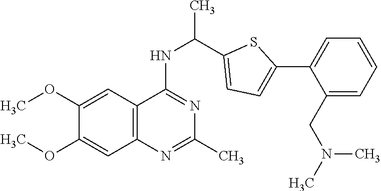 [SMILES: COc1cc2nc(C)nc(NC(C)c3ccc(-c4ccccc4CN(C)C)s3)c2cc1OC]

Enantiopure N-[1-(5-{2-[(dimethylamino)methyl]phenyl}-2-thienyl)ethyl]-6,7-dimethoxy-2-methylquinazolin-4-amine was obtained from racemic N-[1-(5-{2-[(dimethylamino)methyl]phenyl}-2-thienyl)ethyl]-6,7-dimethoxy-2-methylquinazolin-4-amine (described in example 262) by chiral HPLC purification (Method X7). ¹H-NMR (400 MHz, DMSO-d₆): δ [ppm]=8.15 (d, 1H), 7.65 (s, 1H), 7.44-7.40 (m, 1H), 7.38-7.35 (m, 1H), 7.33 7.26 (m, 2H), 7.18 (d, 1H), 7.08 (dd, 1H), 7.05 (s, 1H), 5.96 (quin, 1H), 3.87 (s, 6H), 3.36 (s, 2H), 2.43 (s, 3H), 2.10 (s, 6H), 1.72 (d, 3H). LC-MS (Method 7): m/z: [M+H]⁺=463, Rₜ=0.58 min. [α]D=−158.1°+/−2.00°.

### Example 392

### 2-(4-{[(1R)-1-(3-chlorophenyl)ethyl]amino}-2-methylquinazolin-6-yl)propan-2-ol

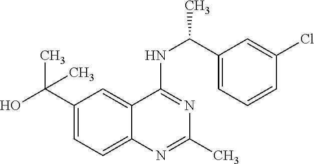 [SMILES: Cc1nc(N[C@H](C)c2cccc(Cl)c2)c2cc(C(C)(C)O)ccc2n1]

To a solution of methyl 4-{[(1R)-1-(3-chlorophenyl)ethyl]amino}-2-methylquinazoline-6-carboxylate (described in example 371, 300 mg, 0.84 mmol) in THF was added at 0° C. methyl magnesium chloride (3.0 M in THF, 1.69 mL, 5.06 mmol). The reaction was allowed to warm to ambient temperature and stirred for three hours at room temperature. More methyl magnesium chloride (3.0 M in THF, 1.69 mL, 5.06 mmol) was added at ambient temperature and the reaction was stirred at RT for another 16 hours. The reaction mixture was poured into saturated ammonium chloride solution and extracted with dichloromethane (3×50 mL). The combined organic layers were evaporated and the residue purified via HPLC chromatography to yield the title compound (108 mg, 35%). ¹H-NMR (400 MHz, DMSO-d₆): δ [ppm]=8.45-8.39 (m, 1H), 8.36 (d, 1H), 8.15 (s, 1H), 7.85 (dd, 1H), 7.56-7.49 (m, 2H), 7.41 (s, 1H), 7.35 (t, 1H), 7.29 (dd, 1H), 5.64 (s, 1H), 5.28-5.13 (m, 1H), 2.37 (s, 3H), 1.59 (d, 3H), 1.53 (s, 6H). LC-MS (Method 7): m/z: [M+H]⁺=356.2, Rₜ=0.83 min.

### Example 393

### 2-(3′-{(1R)-1-[(6,7-di methoxy-2-methylquinazolin-4-yl)am no]ethyl}biphenyl-2-yl)acetamide

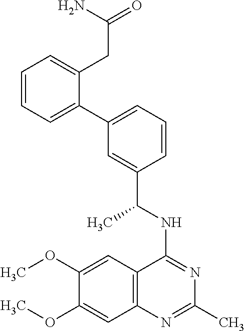

The title compound was prepared in analogy to 4-(3-{(1R)-1-[(6,7-dimethoxy-2-methylquinazolin-4-yl)amino]ethyl}phenyl)pyridin-2-ol (described in example 388) using 2-(aminocarbonylmethyl)phenylboronic acid; pinacol ester (243 mg, 0.93 mmol) and N-[(1R)-1-(3-bromophenyl)ethyl]-6,7-dimethoxy-2-methylquinazolin-4-amine (described in example 191, 150 mg, 373 μmol). Yield: 102 mg (58%). ¹H-NMR (400 MHz, DMSO-d₆): δ [ppm]=8.02-7.96 (m, 1H), 7.69 (s, 1H), 7.49-7.42 (m, 2H), 7.38 (t, 1H), 7.34-7.27 (m, 4H), 7.26-7.18 (m, 2H), 7.02 (s, 1H), 6.91 (br s, 1H), 5.70 (t, 1H), 3.89 (s, 3H), 3.86 (s, 3H), 3.35 (d, 2H), 2.36 (s, 3H), 1.62 (d, 3H). LC-MS (Method 7): m/z: [M+H]⁺=457.2, Rₜ=0.80 min.

### Example 394

### 2-[2-(5-{1-[(6,7-dimethoxy-2-methylquinazolin-4-yl)amino]ethyl}thiophen-2-yl)phenyl]acetamide

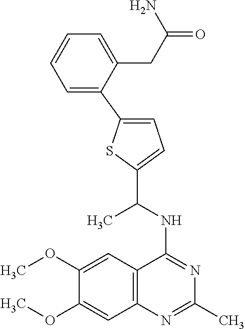

The title compound was prepared in analogy to 4-(3-{(1R)-1-[(6,7-dimethoxy-2-methylquinazolin-4-yl)amino]ethyl}phenyl)pyridin-2-ol (described in example 388) using 2-(aminocarbonylmethyl)phenylboronic acid; pinacol ester (240 mg, 0.92 mmol) and N-[1-(5-bromo-2-thienyl)ethyl]-6,7-dimethoxy-2-methylquinazolin-4-amine (described in example 209, 150 mg, 367 μmol). Yield: 1.1 mg (0.59%). LC-MS (Method 7): m/z: [M+H]⁺=463.2, Rₜ=0.77 min.

### Example 395

### 5-(5-{1-[(6,7-dimethoxy-2-methylquinazolin-4-yl)amino]ethyl}thiophen-2-yl)pyridin-2-ol

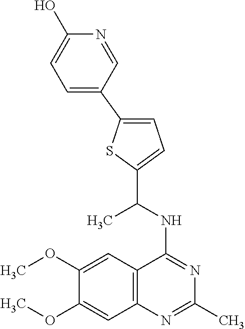

The title compound was prepared in analogy to 4-(3-{(1R)-1-[(6,7-dimethoxy-2-methylquinazolin-4-yl)amino]ethyl}phenyl)pyridin-2-ol (described in example 388) using 6-hydroxypyridine-3-boronic acid; pinacol ester (128 mg, 918 μmol) and N-[1-(5-bromo-2-thienyl)ethyl]-6,7-dimethoxy-2-methylquinazolin-4-amine (described in example 209, 150 mg, 367 μmol). Yield: 16 mg (10%). ¹H-NMR (400 MHz, DMSO-d₆): δ [ppm]=11.71 (br s, 1H), 8.09 (d, 1H), 7.69 (dd, 1H), 7.63 (s, 1H), 7.58 (s, 1H), 7.14 (d, 1H), 7.04 (s, 1H), 7.01 (dd, 1H), 6.37 (d, 1H), 5.89 (t, 1H), 3.87 (s, 6H), 2.43 (s, 3H), 1.68 (d, 3H). LC-MS (Method 7): m/z: [M+H]⁺=423.2, Rₜ=0.65 min.

### Example 396

### N-[(1R)-1-(3-chlorophenyl)ethyl]-6-methoxy-2,8-dimethylquinazolin-4-amine

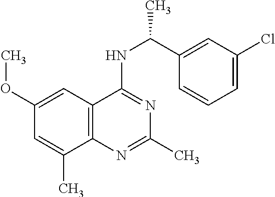 [SMILES: COc1cc(C)c2nc(C)nc(N[C@H](C)c3cccc(Cl)c3)c2c1]

### Step a

### 6-methoxy-2,8-dimethylquinazolin-4(3H)-one

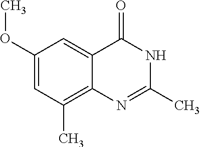

Methyl 2-amino-5-methoxy-3-methylbenzoate, 500 mg (2.6 mmol, commercially available) was dissolved in 20 mL of acetonitrile and dry hydrogen chloride gas was passed into the above solution at room temperature until a clear solution was observed. The resulting mixture was stirred at reflux for overnight. The precipitated solid was collected by filtration, washed with acetonitrile and re-dissolved with water. Saturated aqueous sodium carbonate was added to adjust the pH value to 8 and the precipitated solid was collected by filtration. The filter cake was washed with ice cold water and dried in vacuo to give 212 mg (40%) of the product as a white solid. MS (ESIpos): m/z=205 [M+H]⁺; LC-MS [Method 4, gradient starting with water (0.05% NH₄HCO₃)-Acetonitrile, 10% B]: Rₜ=1.12 min. ¹H-NMR (400 MHz, DMSO-d₆): δ [ppm]=2.31 (s, 3H), 2.44 (s, 3H), 3.80 (s, 3H), 7.21 (s, 1H), 7.28 (s, 1H), 12.10 (br, 1H).

### Step b

### 4-chloro-6-methoxy-2,8-dimethylquinazoline

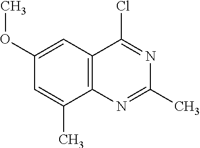

6-Methoxy-2,8-dimethylquinazolin-4(3H)-one, 120 mg (0.60 mmol), was dissolved in 20 mL of phosphoryl trichloride. The resulting mixture was stirred at 110° C. for 3 hours. After cooling to room temperature, the solvent was removed in vacuo and saturated aqueous sodium carbonate was added to adjust the pH value to 8. The mixture was extracted with dichloromethane and the combined organic layers were dried over anhydrous sodium sulfate. The solvent was removed in vacuo to give 97.0 mg (74%) of the product as a yellow solid. MS (ESIpos): m/z=223 [M+H]⁺; LC-MS [Method 4, gradient starting with water (0.05% TFA)-Acetonitrile, 5% B)]: Rₜ=1.11 min.

### Step c

### N-[(1R)-1-(3-chlorophenyl)ethyl]-6-methoxy-2,8-dimethylquinazolin-4-amine

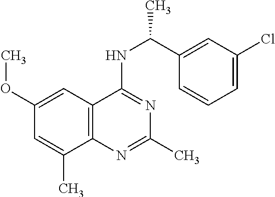 [SMILES: COc1cc(C)c2nc(C)nc(N[C@H](C)c3cccc(Cl)c3)c2c1]

4-Chloro-6-methoxy-2,8-dimethylquinazoline, 60.0 mg (0.30 mmol), and (R)-1-(3-chlorophenyl)ethanamine, 46.1 mg (0.30 mmol, commercially available), were dissolved into 1.0 mL of 2-propanol. The resulting mixture was stirred at 110° C. for 6 hours. After cooling to room temperature, the solvent was removed in vacuo and saturated aqueous sodium carbonate was added to adjust the pH value to 8. The mixture was extracted with dichloromethane and the combined organic layers were dried over anhydrous sodium sulfate. The solvent was removed in vacuo and the residue was purified by preparative HPLC to give 41.3 mg (44%) of the product as a light yellow solid. MS (ESIpos): m/z=342 [M+H]⁺; LC-MS [Method 4, gradient starting with water (0.05% TFA)-Acetonitrile, 5% B]: Rₜ=1.31 min. ¹H-NMR (400 MHz, DMSO-d₆): δ [ppm]=1.59 (d, 3H), 2.39 (s, 3H), 2.49 (s, 3H), 3.88 (s, 3H), 5.60-5.64 (m, 1H), 7.24-7.29 (m, 2H), 7.33-7.42 (m, 2H), 7.49 (s, 1H), 7.58 (s, 1H), 8.10 (d, 1H).

### Example 397

### N-[1-{5-[2-(aminomethyl)-3-chlorophenyl]thiophen-2-yl}ethyl]-6,7-dimethoxy-2-methylquinazolin-4-amine

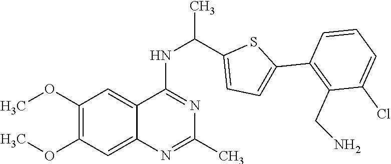 [SMILES: COc1cc2nc(C)nc(NC(C)c3ccc(-c4cccc(Cl)c4CN)s3)c2cc1OC]

This compound was synthesized from tert-butyl [2-chloro-6-(5-{1-[(6,7-dimethoxy-2-methylquinazolin-4-yl)amino]ethyl}thiophen-2-yl)benzyl]carbamate (described in example 450) by the same method as described in example 437 to give 26.2 mg (63%) of the product as an off-white solid. MS (ESIpos): m/z=469 [M+H]⁺. LC-MS [Method 4, Water (0.05% NH₄HCO₃)-Acetonitrile, 10% B]: Rₜ=1.55 min. ¹H-NMR (400 MHz, DMSO-d₆): δ [ppm]=1.72-1.74 (d, 3H), 2.43 (s, 3H), 3.84-3.87 (m, 8H), 5.95-5.98 (m, 1H), 7.05 (s, 1H), 7.12-7.13 (m, 1H), 7.23 (s, 1H), 7.30-7.31 (m, 2H), 7.46-7.47 (m, 1H), 7.65 (s, 1H), 8.16-8.18 (m, 3H).

### Example 398

### N-[1-{5-[2-(aminomethyl)-4-chlorophenyl]thiophen-2-yl}ethyl]-6,7-dimethoxy-2-methylquinazolin-4-amine

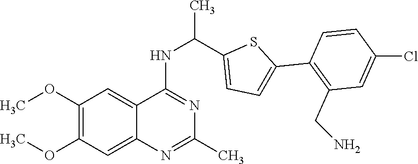 [SMILES: COc1cc2nc(C)nc(NC(C)c3ccc(-c4ccc(Cl)cc4CN)s3)c2cc1OC]

This compound was synthesized from tert-butyl [5-chloro-2-(5-{1-[(6,7-dimethoxy-2-methylquinazolin-4-yl)amino]ethyl}thiophen-2-yl)benzyl]carbamate (described in example 449) by the same method as described in example 437 to give 11.1 mg (33%) of the product as an off-white solid. MS (ESIpos): m/z=469 [M+H]⁺. LC-MS [Method 4, Water (0.05% TFA)-Acetonitrile, 5% B]: Rₜ=1.36 min. ¹H-NMR (400 MHz, DMSO-d₆): δ [ppm]=1.72 (d, 3H), 2.43 (s, 3H), 3.87-3.89 (m, 8H), 5.94-5.97 (m, 1H), 7.05 (s, 1H), 7.08-7.12 (m, 2H), 7.34-7.37 (m, 2H), 7.64-7.68 (m, 2H), 8.16 (d, 1H), 8.23-8.24 (m, 2H).

### Example 399

### N-(1-{5-[2-(aminomethyl)-4-fluorophenyl]-4-methyl-2-thienyl}ethyl)-6,7-dimethoxy-2-methylquinazolin-4-amine

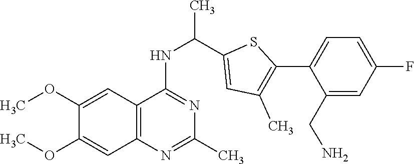 [SMILES: COc1cc2nc(C)nc(NC(C)c3cc(C)c(-c4ccc(F)cc4CN)s3)c2cc1OC]

Under argon, N-[1-(5-bromo-4-methylthiophen-2-yl)ethyl]-6,7-dimethoxy-2-methylquinazolin-4-amine hydrochloride (1:1) (100 mg, 218 μmol, described in example 183), [2-(aminomethyl)-4-fluorophenyl]boronic acid hydrochloride (1:1) (44.8 mg, 218 μmol), K₂CO₃ (120 mg, 872 μmol) and Pd(PPh₃)₄ (25.2 mg, 21.8 μmol) in 1,4-dioxane (2.0 mL) and H₂O (400 μL) were stirred at 110° C. during 36 hours. Additional [2-(aminomethyl)-4-fluorophenyl]boronic acid hydrochloride (1:1) (44.8 mg, 218 μmol) was added and the mixture stirred at 110° C. over the weekend. H₂O was added, the mixture extracted with DCM and the solvent removed in vacuo. Purification by preparative HPLC (basic conditions) gave the title compound as a white solid (9.90 mg, 10%). ¹H-NMR (400 MHz, DMSO-d₆): δ [ppm]=8.11 (d, 1H), 7.64 (s, 1H), 7.42 (dd, 1H), 7.17 (dd, 1H), 7.07-7.01 (m, 3H), 6.93 (d, 1H), 5.91 (sxt, 1H), 3.87 (s, 3H), 3.86 (s, 3H), 2.42 (s, 3H), 1.90 (s, 3H), 1.68 (d, 3H). LC-MS (Method 7): m/z: [M+H]⁺=467, Rₜ=0.60 min.

### Example 400

### N-[1-(5-{2-[(dimethylamino)methyl]phenyl}-4-methyl-2-thienyl)ethyl]-6,7-dimethoxy-2-methylquinazolin-4-amine

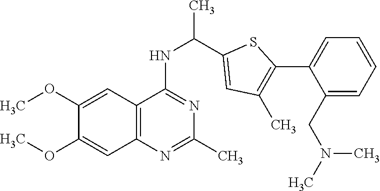 [SMILES: COc1cc2nc(C)nc(NC(C)c3cc(C)c(-c4ccccc4CN(C)C)s3)c2cc1OC]

### Step a

### 2-(5-{1-[(6,7-dimethoxy-2-methylquinazolin-4-yl)amino]ethyl}thiophen-2-yl)benzaldehyde

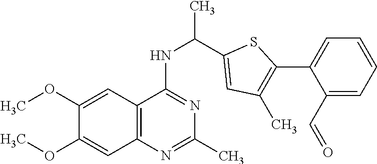 [SMILES: COc1cc2nc(C)nc(NC(C)c3cc(C)c(-c4ccccc4C=O)s3)c2cc1OC]

Under argon, N-[1-(5-bromo-4-methylthiophen-2-yl)ethyl]-6,7-dimethoxy-2-methylquinazolin-4-amine hydrochloride (1:1) (500 mg, 1.09 mmol, described in example 183), 2-formylphenylboronic acid (163 mg, 1.09 mmol), K₂CO₃ (602 mg, 4.36 mmol) and Pd(PPh₃)₄ (126 mg, 109 μmol) in 1,4-dioxane (10 mL) and H₂O (2.0 mL) were stirred at 110° C. overnight. H₂O was added, the mixture extracted with DCM and the solvent removed in vacuo. Purification by preparative HPLC (basic conditions) gave the title compound as an orange solid (340 mg, 63%). ¹H-NM R (400 MHz, DMSO-d₆): δ [ppm]=9.84 (d, 1H), 8.14 (d, 1H), 7.88 (dd, 1H), 7.72 (td, 1H), 7.45 (dd, 1H), 7.05 (s, 1H), 7.03 (d, 1H), 5.95 (quin, 1H), 3.87 (s, 3H), 3.87 (s, 3H), 2.42 (s, 3H), 1.99 (s, 3H), 1.71 (d, 3H). LC-MS (Method 7): m/z: [M+H]⁺=448, Rₜ=0.97 min.

### Step b

### N-[1-(5-{2-[(dimethylamino)methyl]phenyl}-4-methyl-2-thienyl)ethyl]-6,7-dimethoxy-2-methylquinazolin-4-amine

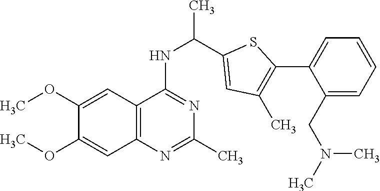 [SMILES: COc1cc2nc(C)nc(NC(C)c3cc(C)c(-c4ccccc4CN(C)C)s3)c2cc1OC]

To 2-(5-{1-[(6,7-dimethoxy-2-methylquinazolin-4-yl)amino]ethyl}thiophen-2-yl)benzaldehyde (50.0 mg, 115 μmol), N-methylmethanamine (110 μl, 2.0 M, 220 μmol) and acetic acid (13 μl, 230 μmol) in 1,2-dichloroethane (1.0 mL) was added NaBH(OAc)₃ (47.4 mg, 223 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a white solid (31.3 mg, 57%). ¹H-NMR (400 MHz, DMSO-d₆): δ [ppm]=8.11 (d, 1H), 7.65 (s, 1H), 7.52 (d, 1H), 7.36 (td, 1H), 7.25 (td, 1H), 7.16 (dd, 1H), 7.04 (s, 1H), 6.91 (d, 1H), 5.92 (quin, 1H), 3.87 (s, 3H), 3.87 (s, 3H), 3.23 (s, 2H), 2.41 (s, 3H), 2.03 (s, 6H), 1.90 (s, 3H), 1.68 (d, 3H). LC-MS (Method 7): m/z: [M+H]⁺=477, Rₜ=0.61 min.

### Example 401

### 2-{[2-(5-{1-[(6,7-dimethoxy-2-methylquinazolin-4-yl)amino]ethyl}-3-methyl-2-thienyl)benzyl](methyl)amino}ethanol

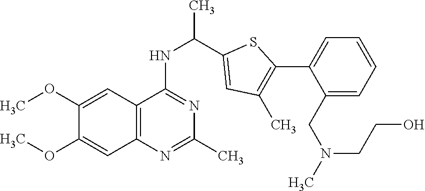 [SMILES: COc1cc2nc(C)nc(NC(C)c3cc(C)c(-c4ccccc4CN(C)CCO)s3)c2cc1OC]

To 2-(5-{1-[(6,7-dimethoxy-2-methylquinazolin-4-yl)amino]ethyl}thiophen-2-yl)benzaldehyde (50.0 mg, 115 μmol, described in example 400 [step a]), 2-(methylamino)ethanol (18 μl, 220 μmol) and acetic acid (13 μl, 230 μmol) in 1,2-dichloroethane (1.0 mL) was added NaBH(OAc)₃ (47.4 mg, 223 μmol) and the solution stirred at room temperature overnight. The reaction was quenched with aqueous NaOH (1.0 M, 1.0 mL), the mixture extracted with DCM and the solvent removed in vacuo. Purification by preparative HPLC (basic conditions) gave the title compound as a white solid (34.6 mg, 55%). ¹H-NMR (400 MHz, DMSO-d₆): δ [ppm]=8.11 (d, 1H), 7.64 (s, 1H), 7.59 (dd, 1H), 7.36 (td, 1H), 7.24 (td, 1H), 7.14 (dd, 1H), 7.04 (s, 1H), 6.91 (d, 1H), 5.92 (quin, 1H), 4.30 (br s, 1H), 3.87 (s, 3H), 3.86 (s, 3H), 3.44-3.37 (m, 3H), 2.42 (s, 3H), 2.30 (t, 2H), 2.04 (s, 3H), 1.90 (s, 3H), 1.68 (d, 3H). LC-MS (Method 7): m/z: [M+H]⁺=507, Rₜ=0.61 min.

### Example 402

### 6,7-di methoxy-2-methyl-N-[1-(4-methyl-5-{2-[(methylamino)methyl]phenyl}thiophen-2-yl)ethyl]quinazolin-4-amine

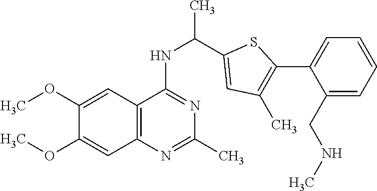 [SMILES: CNCc1ccccc1-c1sc(C(C)Nc2nc(C)nc3cc(OC)c(OC)cc23)cc1C]

Under argon, N-[1-(5-bromo-4-methylthiophen-2-yl)ethyl]-6,7-dimethoxy-2-methylquinazolin-4-amine hydrochloride (1:1) (100 mg, 218 μmol, described in example 183), N-methyl-1-[2 (4,4,5,5-tetramethyl-1,3,2-dioxaborolan-2-yl)phenyl]methanamine (53.9 mg, 218 μmol), K₂CO₃ (120 mg, 872 μmol) and Pd(PPh₃)₄ (25.2 mg, 21.8 μmol) in 1,4-dioxane (2.0 mL) and H₂O (400 μL) were stirred at 110° C. during 36 hours. Additional N-methyl-1-[2-(4,4,5,5-tetramethyl-1,3,2-dioxaborolan-2-yl)phenyl]methanamine (53.9 mg, 218 μmol) was added and the mixture stirred at 110° C. over the weekend. H₂O was added, the mixture extracted with DCM and the solvent removed in vacuo. Purification by preparative HPLC (basic conditions) followed by column chromatography (silica gel, MeOH/EtOAc 0-20% then MeOH) gave the title compound as a white solid (33.5 mg, 33%). ¹H-NMR (400 MHz, DMSO-d₆): δ [ppm]=8.12 (d, 1H), 7.65 (s, 1H), 7.53 (d, 1H), 7.35 (td, 1H), 7.24 (t, 1H), 7.15 (dd, 1H), 7.04 (s, 1H), 6.92 (s, 1H), 5.92 (quin, 1H), 3.87 (s, 6H), 3.48 (s, 2H), 2.42 (s, 3H), 2.14 (s, 3H), 1.92 (s, 3H), 1.68 (d, 3H). LC-MS (Method 7): m/z: [M+H]⁺=463, Rₜ=0.60 min.

### Example 403

### 1-[2-(5-{(1-[(6,7-dimethoxy-2-methylquinazolin-4-yl)amino]ethyl}thiophen-2-yl)phenyl]ethane-1,2-diol

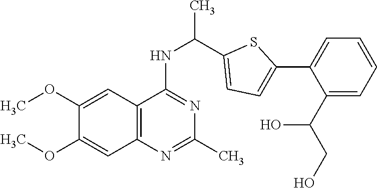 [SMILES: COc1cc2nc(C)nc(NC(C)c3ccc(-c4ccccc4C(O)CO)s3)c2cc1OC]

A solution of N-{1-[5-(2-ethenylphenyl)thiophen-2-yl]ethyl}-6,7-dimethoxy-2-methylquinazolin-4-amine (40.0 mg, 92.7 μmol, described in example 248), osmium tetroxide (94 μl, 2.5% w in tert-butanol, 9.3 μmol) and N-methylmorpholine N-oxide (32.6 mg, 278 μmol) in acetone (1.0 mL) and H₂O (200 μL) was stirred at room temperature overnight. H₂O was added, the mixture extracted with DCM and the solvent removed in vacuo. Purification by preparative HPLC (basic conditions) gave the title compound as a light brown solid (33.5 mg, 33%). ¹H-NMR (400 MHz, DMSO-d₆): δ [ppm]=8.16 (d, 1H), 7.65 (d, 1H), 7.56 (d, 1H), 7.35 (ddd, 1H), 7.28-7.23 (m, 2H), 7.10-7.07 (m, 1H), 7.05 (s, 1H), 7.00 (dd, 1H), 6.02-5.92 (m, 1H), 5.17 (dd, 1H), 4.90-4.81 (m, 1H), 4.74 (td, 1H), 3.87 (s, 6H), 3.46-3.39 (m, 1H), 2.43 (s, 3H), 1.72 (d, 3H). LC-MS (Method 7): m/z: [M+H]⁺=466, Rₜ=0.76 min.

### Example 404

### N-[(1R)-1-(3-chlorophenyl)ethyl]-2,6-dimethylquinazolin-4-amine

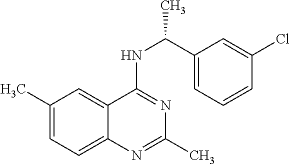 [SMILES: Cc1ccc2nc(C)nc(N[C@H](C)c3cccc(Cl)c3)c2c1]

To a solution of 6-bromo-N-[(1R)-1-(3-chlorophenyl)ethyl]-2-methylquinazolin-4-amine (described in example 363, 100 mg, 265 μmol), methyl boronic acid (20 mg, 319 μmol) and [1,1-bis(diphenylphosphino)ferrocene]dichloropalladium(II), complex with dichloromethane (43.3 mg, 0.053 mmol) in 1,4-dioxane (3.0 mL) was added sodium carbonate solution (1.0 M in water, 0.25 mL) and the reaction was stirred at 80° C. for 4 hours. The reaction was filtered and the filtrate evaporated. The crude product was purified by HPLC chromatography to yield the title compound (10 mg, 11%). ¹H-NMR (400 MHz, DMSO-d₆): δ [ppm]=8.24 (d, 1H), 8.16 (s, 1H), 7.58-7.45 (m, 3H), 7.43-7.38 (m, 1H), 7.34 (t, 1H), 7.30-7.23 (m, 1H), 5.59 (quin, 1H), 2.47 (s, 3H), 2.37 (s, 3H), 1.57 (d, 3H). LC-MS (Method 7): m/z: [M+H]⁺=312.1, Rₜ=0.85 min.

### Example 405

### N-[(1R)-1-(3-chlorophenyl)ethyl]-2-methyl-6-(1H-pyrazol-4-yl)quinazolin-4-amine

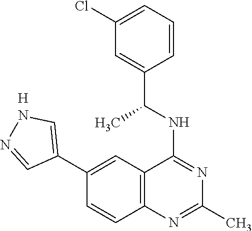 [SMILES: Cc1nc(N[C@H](C)c2cccc(Cl)c2)c2cc(C3=CCN=C3)ccc2n1]

To a solution of 6-bromo-N-[(1R)-1-(3-chlorophenyl)ethyl]-2-methylquinazolin-4-amine (described in example 363, 150 mg, 0.398 mmol), 4-pyrazoleboronic acid 222.7 mg, 1.99 mmol) and [1,1-Bis(diphenylphosphino)ferrocene]dichloropalladium(II), complex with dichloromethane (81.3 mg, 99.0 μmol) in DMF (4.0 mL) was added sodium carbonate solution (2.0 M in water, 0.54 mL) and the reaction was stirred at 80° C. for 3 hours. The reaction was filtered and the filtrate evaporated. The crude product was purified by HPLC chromatography to yield the title compound (62.0 mg, 41%). ¹H-NMR (400 MHz, DMSO-d₆): δ [ppm]=13.04 (br s, 1H), 8.54 (s, 1H), 8.35-7.94 (m, 4H), 7.57 (d, 1H), 7.52 (s, 1H), 7.46-7.41 (m, 1H), 7.37 (t, 1H), 7.32-7.27 (m, 1H), 5.65 (br t, 1H), 2.38 (s, 3H), 1.62 (br d, 3H). LC-MS (Method 7): m/z: [M+H]⁺=364.1, Rₜ=0.80 min.

### Example 406

### N-[(1R)-1-(3-chlorophenyl)ethyl]-2-methyl-6-(1-methyl-1H-pyrazol-4-yl)quinazolin-4-amine

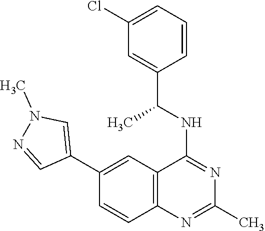 [SMILES: Cc1nc(N[C@H](C)c2cccc(Cl)c2)c2cc(-c3cnn(C)c3)ccc2n1]

To a solution of 6-bromo-N-[(1R)-1-(3-chlorophenyl)ethyl]-2-methylquinazolin-4-amine (described in example 363, 150 mg, 0.398 mmol), 1-methyl-1H-pyrazole-4-boronic acid, pinacol ester (414 mg, 1.99 mmol) and [1,1-bis(diphenylphosphino)-ferrocene]dichloropalladium(II), complex with dichloromethane (81.3 mg, 99.0 μmol) in DMF (4.0 mL) was added sodium carbonate solution (2.0 M in water, 0.54 mL) and the reaction was stirred at 80° C. for 3 hours. The reaction was filtered and the filtrate evaporated. The crude product was purified by HPLC chromatography to yield the title compound (69.0 mg, 45%). ¹H-NMR (400 MHz, DMSO-d₆): δ [ppm]=8.51 (br s, 1H), 8.33-8.16 (m, 2H), 8.06-7.87 (m, 2H), 7.65-7.23 (m, 6H), 5.65 (br s, 1H), 3.91 (br s, 3H), 2.38 (br s, 3H), 1.61 (br d, 3H). LC-MS (Method 7): m/z: [M+H]⁺=378.2, Rₜ=0.84 min.

### Example 407

### N-[(1R)-1-(3-chlorophenyl)ethyl]-6-cyclopropyl-2-methylquinazolin-4-amine

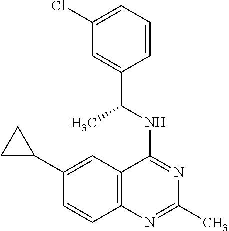 [SMILES: Cc1nc(N[C@H](C)c2cccc(Cl)c2)c2cc(C3CC3)ccc2n1]

To a solution of 6-bromo-N-[(1R)-1-(3-chlorophenyl)ethyl]-2-methylquinazolin-4-amine (described in example 363, 150 mg, 0.398 mmol), cyclopropyl boronic acid (171 mg, 1.99 mmol) and [1,1-bis(diphenylphosphino)ferrocene]dichloropalladium(II), complex with dichloromethane (81.3 mg, 99.0 μmol) in DMF (4.0 mL) was added sodium carbonate solution (2.0 M in water, 0.54 mL) and the reaction was stirred at 80° C. for 3 hours. The reaction was filtered and the filtrate evaporated. The crude product was purified by HPLC chromatography to yield the title compound (20 mg, 14%). LC-MS (Method 7): m/z: [M+H]⁺=338.1, Rₜ=0.93 min.

### Example 408

### tert-butyl [3-chloro-2-(5-{1-[(6,7-dimethoxy-2-methylquinazolin-4-yl)amino]ethyl}thiophen-2-yl)benzyl]carbamate

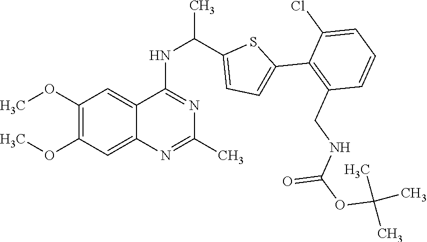 [SMILES: COc1cc2nc(C)nc(NC(C)c3ccc(-c4c(Cl)cccc4CNC(=O)OC(C)(C)C)s3)c2cc1OC]

### Step a

### 1-(2-bromo-3-chlorophenyl)methanamine

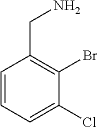

To 25 mL of borane tetrahydrofuran complex (1.0 M in THF) was added a solution of 2-bromo-3-chlorobenzonitrile, 1.00 g (4.6 mmol, commercially available), in 5.0 mL of THF at 0° C. and the resulting mixture was stirred at reflux for 15 hours under nitrogen atmosphere. After cooling to room temperature, methanol and hydrochloric acid were added dropwise to the above solution at 0° C. and the resulting mixture was stirred at room temperature for 30 minutes.

Sodium hydroxide solution (1.0 M) was added to adjust the pH value to 7 and the resulting mixture was extracted with dichloromethane. The combined organic layers were dried over sodium sulfate and concentrated in vacuo to give 610 mg (60%) of the product as a light yellow oil. MS (ESIpos): m/z=220 [M+H]⁺. LC-MS [Method 4, Water (0.1% HCOOH)-Acetonitrile, 10% B]: Rₜ=0.51 min.

### Step b

### tert-butyl (2-bromo-3-chlorobenzyl)carbamate

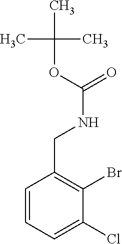 [SMILES: CC(C)(C)OC(=O)NCc1cccc(Cl)c1Br]

This compound was synthesized by the same method as described in example 428 (step a) to give 720 mg (81%) of the product as an off-white solid. MS (ESIpos): m/z=320 [M+H]⁺. LC-MS [Method 4, Water (0.05% TFA)-Acetonitrile, 5% B]: Rₜ=1.18 min.

### Step c

### tert-butyl [3-chloro-2-(4,4,5,5-tetramethyl-1,3,2-dioxaborolan-2-yl)benzyl]carbamate

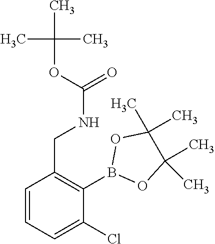 [SMILES: CC(C)(C)OC(=O)NCc1cccc(Cl)c1B1OC(C)(C)C(C)(C)O1]

This compound was synthesized by the same method as described in example 428 (step b) to give 408 mg (49%) of the product as a light yellow oil. MS (ESIpos): m/z=368 [M+H]⁺. LC-MS [Method 4, Water (0.05% TFA)-Acetonitrile, 5% B]: Rₜ=1.25 min.

### Step d

### tert-butyl [3-chloro-2-(5-{1-[(6,7-dimethoxy-2-methylquinazolin-4-yl)amino]ethyl}thiophen-2-yl)benzyl]carbamate

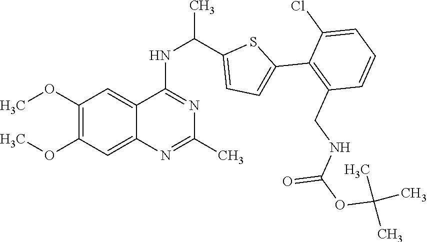 [SMILES: COc1cc2nc(C)nc(NC(C)c3ccc(-c4c(Cl)cccc4CNC(=O)OC(C)(C)C)s3)c2cc1OC]

This compound was synthesized by the same method as described in example 429 to give 160 mg (51%) of the product as an off-white solid. MS (ESIpos): m/z=569 [M+H]⁺. LC-MS [Method 4, Water (0.05% TFA)-Acetonitrile, 5% B]: Rₜ=1.50 min. ¹H-NMR (400 MHz, DMSO-d₆): δ [ppm]=1.37 (s, 9H), 1.73 (d, 3H), 2.43 (s, 3H), 3.87 (s, 6H), 3.94 (d, 2H), 5.97-6.01 (m, 1H), 6.86 6.87 (m, 1H), 7.05 (s, 1H), 7.10 (d, 1H), 7.23-7.34 (m, 2H), 7.39-7.45 (m, 2H), 7.66 (s, 1H), 8.19 (d, 1H).

### Example 409

### N-[1-{5-[2-(aminomethyl)-6-chlorophenyl]thiophen-2-yl}ethyl]-6,7-dimethoxy-2-methylquinazolin-4-amine

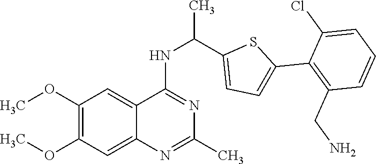 [SMILES: COc1cc2nc(C)nc(NC(C)c3ccc(-c4c(Cl)cccc4CN)s3)c2cc1OC]

This compound was synthesized from tert-butyl [3-chloro-2-(5-{1-[(6,7-dimethoxy-2-methylquinazolin-4-yl)amino]ethyl}thiophen-2-yl)benzyl]carbamate (described in example 408) by the same method as described in example 430 to give 18.2 mg (33%) of the product as an off-white solid. MS (ESIpos): m/z=469 [M+H]⁺. LC-MS [Method 4, Water (0.05% TFA)-Acetonitrile, 5% B]: Rₜ=1.05 min. ¹H-NMR (400 MHz, DMSO-d₆): δ [ppm]=1.73 (d, 3H), 2.33 (s, 3H), 3.50 (s, 2H), 3.87 (s, 6H), 5.95-6.00 (m, 1H), 6.87 (s, 1H), 7.05-7.09 (m, 2H), 7.39-7.43 (m, 2H), 7.52-7.54 (m, 1H), 7.55-7.66 (m, 1H), 8.19 (d, 1H).

### Example 410

### 4-(5-{1-[(6,7-dimethoxy-2-methylquinazolin-4-yl)amino]ethyl}-2-thienyl)pyridin-2-ol

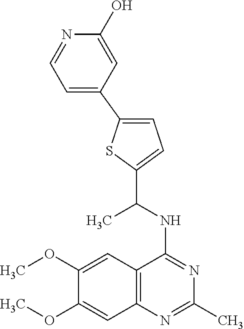

The title compound was prepared in analogy to 4-(3-{(1R)-1-[(6,7-dimethoxy-2-methylquinazolin-4-yl)amino]ethyl}phenyl)pyridin-2-ol (described in example 388) using 2-hydroxypyridine-4-boronic acid, pinacol ester (203 mg, 0.918 mmol) and N-[1-(5-bromo-2-thienyl)ethyl]-6,7-dimethoxy-2-methylquinazolin-4-amine (150 mg, 0.367 mmol, described in example 209). Yield: 3.3 mg (1.7%). LC-MS (Method 7): m/z: [M+H]⁺=423.1, Rₜ=0.63 min.

### Example 411

### 4-[2-(5-{1-[(6,7-dimethoxy-2-methylquinazolin-4-yl)amino]ethyl}thiophen-2-yl)phenyl]azetidin-2-one

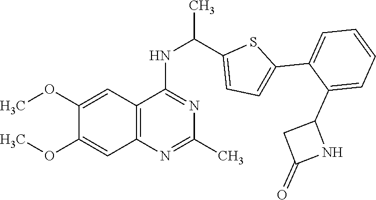 [SMILES: COc1cc2nc(C)nc(NC(C)c3ccc(-c4ccccc4C4CC(=O)N4)s3)c2cc1OC]

### Step a

### N-{[2-(5-{1-[(6,7-dimethoxy-2-methylquinazolin-4-yl)amino]ethyl}-2-thienyl)-phenyl]methylene}-2-methylpropane-2-sulfinamide

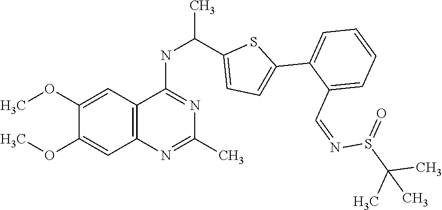

A solution of 2-(5-{1-[(6,7-dimethoxy-2-methylquinazolin-4-yl)amino]ethyl}thiophen-2-yl)benzaldehyde (500 mg, 1.15 mmol, described in example 179 [step b]), 2-methylpropane-2-sulfinamide (168 mg, 1.38 mmol) and Ti(OEt)₄ (480 μl, 2.3 mmol) in THE (1.0 mL) was stirred at room temperature overnight. The reaction was quenched with brine (0.7 mL), diluted with EtOAc and filtered over a celite plug. Purification by column chromatography (silica gel, MeOH/EtOAc 0-20% then MeOH) gave the title compound which was used directly in step b. LC-MS (Method 10): m/z: [M+H]⁺=537, Rₜ=1.45 min.

### Step b

### 4-[2-(5-{1-[(6,7-dimethoxy-2-methylquinazolin-4-yl)amino]ethyl}thiophen-2-yl)phenyl]azetidin-2-one

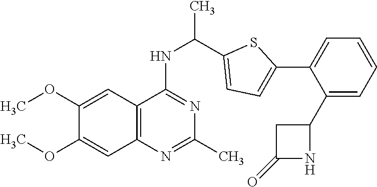 [SMILES: COc1cc2nc(C)nc(NC(C)c3ccc(-c4ccccc4C4CC(=O)N4)s3)c2cc1OC]

A solution of N-{[2-(5-{1-[(6,7-dimethoxy-2-methylquinazolin-4-yl)amino]ethyl}thiophen-2-yl)phenyl]methylidene}-2-methylpropane-2-sulfinamide (50.0 mg, 93.2 μmol) and malonic acid (19.4 mg, 186 μmol) in acetic acid (1.0 mL) was stirred at 120° C. overnight. The solvent was removed in vacuo. Purification by preparative HPLC (basic conditions) gave the title compound as a white solid (9.60 mg, 22%). ¹H-NMR (400 MHz, DMSO-d₆): δ [ppm]=8.19 (d, 1H), 7.83 (d, 1H), 7.77 (d, 1H), 7.64 (s, 1H), 7.45-7.37 (m, 3H), 7.16 (dd, 1H), 7.05 (s, 1H), 6.92 (d, 1H), 6.49 (d, 1H), 5.98 (quin, 1H), 3.87 (s, 3H), 3.87 (s, 3H), 2.44 (s, 3H), 1.73 (d, 3H). LC-MS (Method 7): m/z: [M+H]⁺=475, Rₜ=0.89 min.

### Example 412

### N-[(1R)-1-(3-chlorophenyl)ethyl]-6-methoxy-2,7-dimethylquinazolin-4-amine

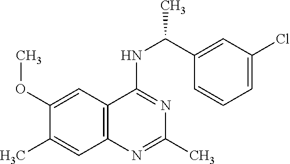 [SMILES: COc1cc2c(N[C@H](C)c3cccc(Cl)c3)nc(C)nc2cc1C]

### Step a

### methyl 5-methoxy-4-methyl-2-nitrobenzoate; PROS589-1

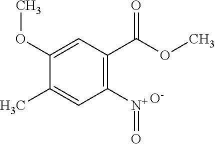

5-Methoxy-4-methyl-2-nitrobenzoic acid, 500 mg (2.4 mmol), and 0.5 mL of conc. sulfuric acid were dissolved in 20 mL of methanol. The resulting mixture was stirred at 65° C. overnight. After cooling to room temperature, the solvent was removed in vacuo and saturated aqueous sodium carbonate was added to adjust the pH value to 8. The resulting mixture was extracted with dichloromethane and the combined organic layers were dried over anhydrous sodium sulfate. The solvent was removed in vacuo and the residue was purified with silica gel column chromatography to give 465 mg (87%) of the product as a light yellow solid. MS (ESIpos): m/z=226 [M+H]⁺; LC-MS [Method 4, gradient starting with water (0.05% TFA)-Acetonitrile, 5% B]: Rₜ=1.03 min.

### Step b

### methyl 2-amino-5-methoxy-4-methylbenzoate

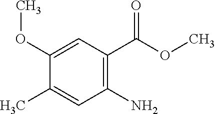

Methyl 5-methoxy-4-methyl-2-nitrobenzoate, 465 mg (2.1 mmol), and 0.1 g of palladium/carbon (10%), were added into 10 mL of methanol. The resulting slurry was stirred at room temperature for overnight under hydrogen atmosphere (3 atm). Catalyst was removed by filtration and the filtrate was concentrated in vacuo to give 393 mg (94%) of the product as a light yellow solid. MS (ESIpos): m/z=196 [M+H]⁺; LC-MS [Method 4, gradient starting with water (0.05% TFA)-Acetonitrile, 5% B]: Rₜ=0.82 min.

### Step c

### 6-methoxy-2,7-dimethylquinazolin-4(3H)-one

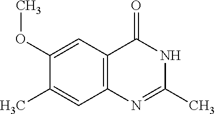

Methyl 2-amino-5-methoxy-4-methylbenzoate, 393 mg (2.0 mmol), was dissolved into 20 mL of acetonitrile and dry hydrogen chloride gas was introduced into the above solution at room temperature until a clear solution was observed. Then the resulting mixture was stirred at reflux for overnight. The precipitated solid was collected by filtration, washed with acetonitrile and re-dissolved with water. Saturated aqueous sodium carbonate was added to adjust the pH value to 8 and the precipitated solid was collected by filtration, washed with ice cold water and dried in vacuo to give 308 mg (74%) of the product as a white solid. MS (ESIpos): m/z=205 [M+H]⁺; LC-MS [Method 4, gradient starting with water (0.05% TFA)-Acetonitrile, 5% B]: Rₜ=0.76 min. ¹H-NMR (400 MHz, DMSO-d₆): δ [ppm]=2.25 (s, 3H), 2.41 (s, 3H), 3.86 (s, 3H), 7.37 (s, 1H), 7.38 (s, 1H), 12.02 (br, 1H).

### Step d

### 4-chloro-6-methoxy-2,7-dimethylquinazoline

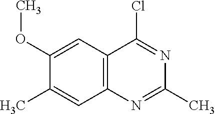

6-Methoxy-2,7-dimethylquinazolin-4(3H)-one, 130 mg (0.6 mmol), was dissolved into 10 mL of phosphoryl trichloride. The resulting mixture was stirred at 110° C. for 3 hours. After cooling to room temperature, the solvent was removed in vacuo and saturated aqueous sodium carbonate was added to adjust the pH value to 8. The mixture was extracted with dichloromethane and the combined organic layers were dried over anhydrous sodium sulfate. The solvent was removed in vacuo to give 121 mg (68%) of the product, as a yellow solid. MS (ESIpos): m/z=223 [M+H]⁺; LC-MS [Method 4, gradient starting with water (0.05% TFA)-Acetonitrile, 5% B]: Rₜ=1.01 min.

### Step e

### N-[(1R)-1-(3-chlorophenyl)ethyl]-6-methoxy-2,7-dimethylquinazolin-4-amine

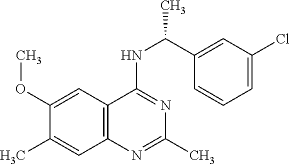 [SMILES: COc1cc2c(N[C@H](C)c3cccc(Cl)c3)nc(C)nc2cc1C]

4-Chloro-6-methoxy-2,7-dimethylquinazoline, 60.0 mg (0.3 mmol), and (R)-1-(3-chlorophenyl)ethanamine, 46.1 mg (0.3 mmol), were dissolved in 1.0 mL of 2-propanol. The resulting mixture was stirred at 110° C. for 6 hours. After cooling to room temperature, the solvent was removed in vacuo and saturated aqueous sodium carbonate was added to adjust the pH value to 8. The mixture was extracted with dichloromethane and the combined organic layers were dried over anhydrous sodium sulfate. The solvent was removed in vacuo and the residue was purified by preparative HPLC to give 44.5 mg (48%) of the product, as a white solid. MS (ESIpos): m/z=342 [M+H]⁺; LC-MS [Method 4, gradient starting with water (0.05% TFA)-Acetonitrile, 5% B]: Rₜ=1.33 min. ¹H-NMR (300 MHz, DMSO-d₆): δ [ppm]=1.61 (d, 3H), 2.29 (s, 3H), 2.36 (s, 3H), 3.96 (s, 3H), 5.59-5.69 (m, 1H), 7.28 (d, 1H), 7.34-7.41 (m, 3H), 7.43 (s, 1H), 7.68 (s, 1H), 8.16 (s, 1H).

### Example 413

### 4-(5-{1-[(6,7-dimethoxy-2-methylquinazolin-4-yl)amino]ethyl}thiophen-2-yl)-3-[(dimethylamino)methyl]benzonitrile

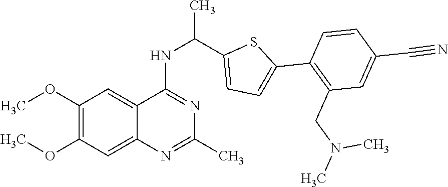 [SMILES: COc1cc2nc(C)nc(NC(C)c3ccc(-c4ccc(C#N)cc4CN(C)C)s3)c2cc1OC]

### Step a

### 3-methyl-4-(4,4,5,5-tetramethyl-1,3,2-dioxaborolan-2-yl)benzonitrile

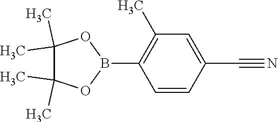 [SMILES: Cc1cc(C#N)ccc1B1OC(C)(C)C(C)(C)O1]

4-Bromo-3-methylbenzonitrile, 500 mg (2.55 mmol), 4,4,4′,4′,5,5,5′,5′-octamethyl-2,2′-bi(1,3,2-dioxaborolane), 712 mg (2.81 mmol), potassium acetate, 751 mg (7.65 mmol), 1,1′-bis(diphenylphosphino)ferrocene-palladium(II)dichloride dichloromethane complex, 208 mg (0.26 mmol), were added into 5.0 mL of N,N-dimethylformamide. The resulting mixture was stirred at 100° C. for 24 hours under nitrogen atmosphere. After cooling to room temperature, water was added and the resulting mixture was extracted with ethyl acetate. The combined organic layers were dried over anhydrous sodium sulfate and the solvent was removed in vacuo. The residue was purified by preparative TLC to give 480 mg (77%) of the product as a green solid. ¹H-NMR (400 MHz, DMSO): δ [ppm]=1.12 (s, 12H), 2.46 (s, 3H), 7.57-7.58 (d, 1H), 7.62 (s, 1H), 7.69-7.72 (d, 1H).

### Step b

### 3-(bromomethyl)-4-(4,4,5,5-tetramethyl-1,3,2-dioxaborolan-2-yl)benzonitrile

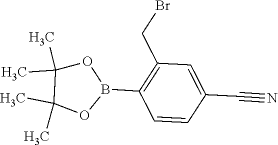 [SMILES: CC1(C)OB(c2ccc(C#N)cc2CBr)OC1(C)C]

3-Methyl-4-(4,4,5,5-tetramethyl-1,3,2-dioxaborolan-2-yl)benzonitrile, 200 mg (0.82 mmol), was dissolved into 2 mL of 1,2-dichloroethane. Then 2,2′-azobisisobutyronitrile, 14 mg (0.08 mmol), and N-bromosuccinimide, 176 mg (0.99 mmol), were added into the above solution at reflux and the resulting mixture was stirred at this temperature for 12 hours under nitrogen atmosphere. After cooling to room temperature, the solvent was removed in vacuo and the residue was purified by preparative TLC to give 140 mg (52%) of the product as a yellow solid. ¹H-NMR (400 MHz, DMSO): δ [ppm] 1.27 (s, 12H), 4.90 (s, 2H), 7.73-7.78 (m, 2H), 7.94 (s, 1H).

### Step c

### 3-((dimethylamino)methyl)-4-(4,4,5,5-tetramethyl-1,3,2-dioxaborolan-2-yl)benzonitrile

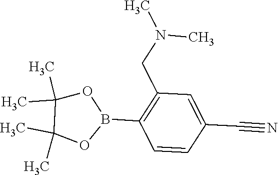 [SMILES: CN(C)Cc1cc(C#N)ccc1B1OC(C)(C)C(C)(C)O1]

3-(Bromomethyl)-4-(4,4,5,5-tetramethyl-1,3,2-dioxaborolan-2-yl)benzonitrile, 100 mg (0.31 mmol), was dissolved into 1.7 mL of dimethylamine/THF (2.0 M) solution. The resulting solution was stirred at room temperature for 12 hours. The solvent was removed in vacuo to give 90 mg (crude) of the product as a yellow solid. It was used directly for next step without further purification. MS (ESIpos): m/z=205 (M of bonic acid+H)⁺. LC-MS [Method 4, gradient starting with water (0.05% TFA)-Acetonitrile, 5% B]: Rₜ=0.52 min.

### Step d

### 4-(5-{1-[(6,7-dimethoxy-2-methylquinazolin-4-yl)amino]ethyl}thiophen-2-yl)-3-[(dimethylamino)methyl]benzonitrile

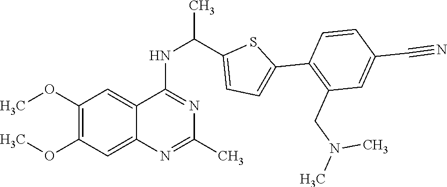 [SMILES: COc1cc2nc(C)nc(NC(C)c3ccc(-c4ccc(C#N)cc4CN(C)C)s3)c2cc1OC]

N-(1-(5-bromothiophen-2-yl)ethyl)-6,7-dimethoxy-2-methylquinazolin-4-amine, 50 mg (0.15 mmol), 3-((dimethylamino)methyl)-4-(4,4,5,5-tetramethyl-1,3,2-dioxaborolan-2-yl)benzonitrile, 87 mg (0.30 mmol), tetrakis(triphenylphosphine)palladium, 18 mg (0.02 mmol), and potassium carbonate, 84 mg (0.61 mmol), were added into 1.4 mL of 1,4-dioxane/water (v:v=5:2). The resulting mixture was stirred at 100° C. After cooling to room temperature, water was added and the resulting mixture was extracted with ethyl acetate. The combined organic layers were dried over anhydrous sodium sulfate and the solvent was removed in vacuo. The residue was purified by preparative HPLC to give 5.8 mg (10%) of the final product as a white solid. MS (ESIpos): m/z=488 [M+H]⁺; LC-MS [Method 4, gradient starting with water (0.05% TFA)-Acetonitrile, 5% B]: Rₜ=0.96 min; ¹H-NMR (400 MHz, DMSO-d₆): δ [ppm] 1.70-1.72 (d, 3H), 2.10 (s, 6H), 2.41 (s, 3H), 3.31 (s, 2H), 3.85 (s, 6H), 5.93-5.97 (m, 1H), 7.04 (s, 1H), 7.13-7.14 (d, 1H), 7.31-7.32 (d, 1H), 7.56-7.58 (d, 1H), 7.63 (s, 1H), 7.72-7.74 (d, 1H), 7.84 (s, 1H), 8.14 8.16 (d, 1H).

### Example 414

### N-[1-(5-bromothiophen-2-yl)ethyl]-6-[3-(dimethylamino)pyrrolidin-1-yl]-2-methylquinazolin-4-amine

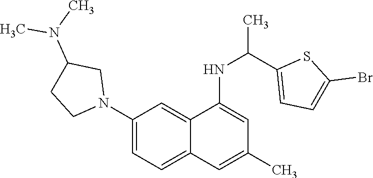 [SMILES: Cc1cc(NC(C)c2ccc(Br)s2)c2cc(N3CCC(N(C)C)C3)ccc2c1]

### Step a

### methyl 5-(3-(dimethylamino)pyrrolidin-1-yl)-2-nitrobenzoate

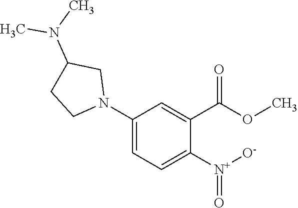

This compound was synthesized by the same method as described in example 419 (step b) to give 580 mg (71%) of the product as a yellow solid. MS (ESIpos): m/z=294 [M+H]⁺; LC-MS [Method 4, gradient starting with water (0.05% NH₄HCO₃)-Acetonitrile, 10% B)]: Rₜ=1.63 min.

### Step b

### methyl 2-amino-5-(3-(dimethylamino)pyrrolidin-1-yl)benzoate

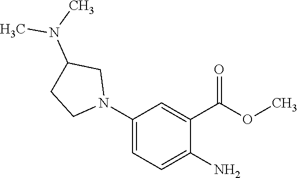

This compound was synthesized by the same method as described in example 419 (step c) to give 470 mg (81%) of the product as a grey solid. MS (ESIpos): m/z=264 [M+H]⁺; LC-MS [Method 4, gradient starting with water (0.05% NH₄HCO₃)-Acetonitrile, 10% B]: Rₜ=1.41 min.

### Step c

### 6-(3-(dimethylamino)pyrrolidin-1-yl)-2-methylquinazolin-4(3H)-one

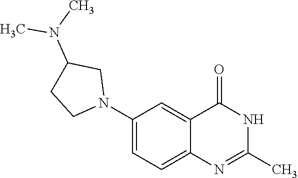

This compound was synthesized by the same method as described in example 419 (step d) to give 305.6 mg (71%) of the product as a white solid. MS (ESIpos): m/z=273 [M+H]⁺; LC-MS [Method 4, gradient starting with water (0.05% NH₄HCO₃)-Acetonitrile, 10% B]: Rₜ=1.15 min. ¹H-NMR (300 MHz, DMSO-d₆): δ [ppm]=1.80-1.90 (m, 1H), 2.15-2.17 (m, 1H), 2.21 (s, 3H), 2.22 (s, 3H), 2.28 (s, 3H), 2.79-2.84 (m, 1H), 3.07-3.13 (m, 1H), 3.26-3.33 (m, 1H), 3.41-3.54 (m, 2H), 6.97 (s, 1H), 7.08 (d, 1H), 7.42 (d, 1H), 11.89 (s, 1H).

### Step d

### 1-(4-chloro-2-methylquinazolin-6-yl)-N,N-dimethylpyrrolidin-3-amine

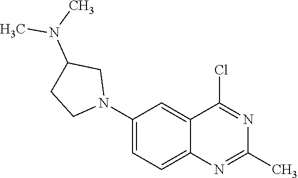

This compound was synthesized by the same method as described in example 419 (step e) to give 530 mg (81%) of the product as a white solid. MS (ESIpos): m/z=291 [M+H]⁺; LC-MS [Method 4, gradient starting with water (0.05% NH₄HCO₃)-Acetonitrile, 10% B]: Rₜ=1.60 min.

### Step e

### N-[1-(5-bromothiophen-2-yl)ethyl]-6-[3-(dimethylamino)pyrrolidin-1-yl]-2-methylquinazolin-4-amine

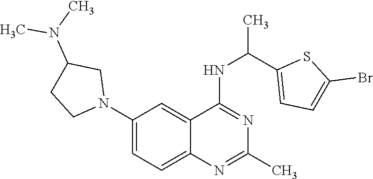 [SMILES: Cc1nc(NC(C)c2ccc(Br)s2)c2cc(N3CCC(N(C)C)C3)ccc2n1]

This compound was synthesized by the same method as described in example 419 (step f) to give 180 mg (40%) of the product as a yellow solid. MS (ESIpos): m/z=460 [M+H]⁺; LC-MS [Method 4, gradient starting with water (0.05% NH₄HCO₃)-Acetonitrile, 10% B]: Rₜ=2.05 min. ¹H-NMR (400 MHz, DMSO-d₆): δ [ppm]=1.67 (d, 3H), 1.82-1.88 (m, 1H), 2.18-2.19 (m, 1H), 2.21 (s, 3H), 2.23 (s, 3H), 2.41 (s, 3H), 2.83-2.87 (m, 1H), 3.10-3.16 (m, 1H), 3.32-3.34 (m, 1H), 3.45-3.56 (m, 2H), 5.79-5.83 (m, 1H), 6.91 (s, 1H), 7.05 (d, 1H), 7.06 (d, 1H), 7.16 (d, 1H), 7.47 (d, 1H), 8.03 (d, 1H).

### Example 415

### N-[1-(5-{2-[(dimethylamino)methyl]phenyl}thiophen-2-yl)ethyl]-2-methyl-6-(pyrrolidin-1-yl)quinazolin-4-amine

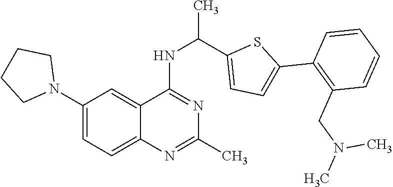 [SMILES: Cc1nc(NC(C)c2ccc(-c3ccccc3CN(C)C)s2)c2cc(N3CCCC3)ccc2n1]

N-(1-(5-bromothiophen-2-yl)ethyl)-2-methyl-6-(pyrrolidin-1-yl)quinazolin-4-amine formate, 30 mg (0.14 mmol, described in example 419), 2-((dimethylamino)methyl)phenylboronic acid, 38.6 mg (0.2 mmol), tetrakis(triphenylphosphine)palladium(0), 8.3 mg (0.01 mmol), and potassium carbonate, 39.7 mg (0.3 mmol), were added into 1.2 mL of 1,4-dioxane/water (v:v=5:1). The resulting mixture was stirred at 100° C. for 16 hours under nitrogen atmosphere. After cooling to room temperature, the solvent was removed in vacuo the residue was purified by preparative HPLC to give 5.2 mg of the product as a yellow solid. MS (ESIpos): m/z=473 [M+H]⁺; LC-MS [Method 4, starting with gradient water (0.05% NH₄HCO₃)-Acetonitrile, 50% B]: Rₜ=1.38 min. ¹H-NMR (400 MHz, DMSO-d₆): δ [ppm]=1.73 (d, 3H), 1.99-2.01 (m, 4H), 2.12 (s, 6H), 2.42 (s, 3H), 3.32-3.33 (m, 4H), 3.37 (s, 2H), 5.96-6.02 (m, 1H), 7.09-7.10 (m, 2H), 7.18 7.19 (m, 2H), 7.30-7.31 (m, 3H), 7.46 (d, 1H), 7.49 (s, 1H), 8.13 (d, 1H).

### Example 416

### N-[1-(5-bromothiophen-2-yl)ethyl]-2-methyl-6-(4-methylpiperazin-1-yl)quinazolin-4-amine

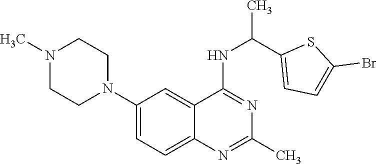 [SMILES: Cc1nc(NC(C)c2ccc(Br)s2)c2cc(N3CCN(C)CC3)ccc2n1]

### Step a

### methyl 5-(4-methylpiperazin-1-yl)-2-nitrobenzoate

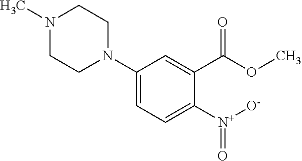

This compound was synthesized by the same method as described in example 419 (step b) to give 0.58 g (83%) of the product as a yellow solid. MS (ESIpos): m/z=280 [M+H]⁺; LC-MS [Method 4, Water (0.05% NH₄HCO₃)-Acetonitrile, 10% B]: Rₜ=1.54 min.

### Step b

### methyl 2-amino-5-(4-methylpiperazin-1-yl)benzoate

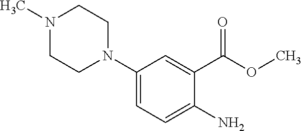

This compound was synthesized by the same method as described in example 419 (step c) to give 0.48 g (83%) of the product as a grey solid. MS (ESIpos): m/z=250 [M+H]⁺; LC-MS [Method 4, gradient starting with water (0.05% NH₄HCO₃)-Acetonitrile, 10% B]: Rₜ=1.22 min.

### Step c:

### 2-methyl-6-(4-methylpiperazin-1-yl)quinazolin-4(3H)-one

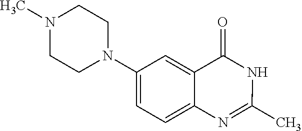

This compound was synthesized by the same method as described in example 419 (step d) to give 199.1 mg (40%) of the product as a white solid. MS (ESIpos): m/z=259 [M+H]⁺; LC-MS [Method 4, gradient starting with water (0.05% NH₄HCO₃)-Acetonitrile, 10% B]: Rₜ=1.03 min. ¹H-NMR (300 MHz, DMSO-d₆): δ [ppm]=2.23 (s, 3H), 2.30 (s, 3H), 2.46-2.50 (m, 4H), 3.19-3.22 (m, 4H), 7.36 (s, 1H), 7.42-7.53 (m, 2H), 12.01 (s, 1H).

### Step d

### 4-chloro-2-methyl-6-(4-methyl piperazin-1-yl)quinazoline

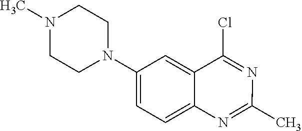

This compound was synthesized by the same method as described in example 419 (step e) to give 420 mg (78%) of the product as a white solid. MS (ESIpos): m/z=277 [M+H]⁺; LC-MS [Method 4, gradient starting with water (0.05% TFA)-Acetonitrile, 5% B]: Rₜ=0.65 min.

### Step e

### N-[1-(5-bromothiophen-2-yl)ethyl]-2-methyl-6-(4-methylpiperazin-1-yl)quinazolin-4-amine

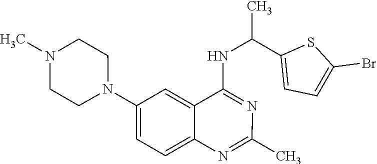 [SMILES: Cc1nc(NC(C)c2ccc(Br)s2)c2cc(N3CCN(C)CC3)ccc2n1]

This compound was synthesized by the same method as described in example 419 (step f) to give 130 mg (40%) of the product as a light yellow solid. MS (ESIpos): m/z=446 [M+H]⁺; LC-MS [Method 4, gradient starting with water (0.05% NH₄HCO₃)-Acetonitrile, 10% B]: Rₜ=1.94 min. ¹H-NMR (300 MHz, DMSO-d₆): δ [ppm]=1.67 (d, 3H), 2.24 (s, 3H), 2.43 (s, 3H), 2.49-2.51 (m, 4H), 3.22-3.26 (m, 4H), 5.78-5.83 (m, 1H), 6.92 (d, 1H), 7.06 (d, 1H), 7.46 (s, 1H), 7.50-7.54 (m, 2H), 8.19 (d, 1H).

### Example 417

### N-[1-(5-{2-[(dimethylamino)methyl]phenyl}thiophen-2-yl)ethyl]-2-methyl-6-(4-methylpiperazin-1-yl)quinazolin-4-amine

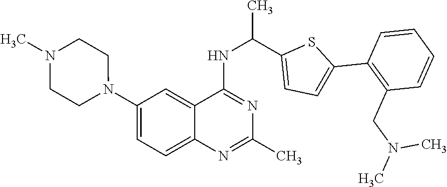 [SMILES: Cc1nc(NC(C)c2ccc(-c3ccccc3CN(C)C)s2)c2cc(N3CCN(C)CC3)ccc2n1]

This compound was synthesized from N-[1-(5-bromothiophen-2-yl)ethyl]-2-methyl-6-(4-methylpiperazin-1-yl)quinazolin-4-amine (described in example 416) by the same method as described in example 415 to give 45.3 mg (67%) of the product as a yellow solid. MS (ESIpos): m/z=501 [M+H]⁺; LC-MS [Method 4, gradient starting with water (0.05% TFA)-Acetonitrile, 5% B]: Rₜ=0.85 min. ¹H-NMR (300 MHz, DMSO-d₆): δ [ppm]=1.72 (d, 3H), 2.08 (s, 3H), 2.10 (s, 3H), 2.24 (s, 3H), 2.43 (s, 3H), 2.47-2.49 (m, 4H), 3.24-3.26 (m, 4H), 3.35-3.36 (m, 2H), 5.96-6.01 (m, 1H), 7.08 (d, 1H), 7.18 (d, 1H), 7.29-7.49 (m, 4H), 7.51-7.54 (m, 3H), 8.26 (d, 1H).

### Example 418

### N-[1-(5-{2-[(dimethylamino)methyl]phenyl}thiophen-2-yl)ethyl]-6-[3-(dimethylamino)pyrrolidin-1-yl]-2-methylquinazolin-4-amine

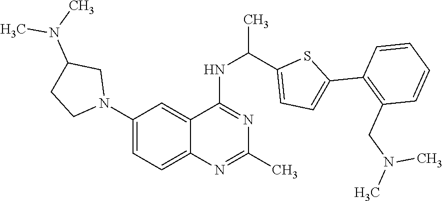 [SMILES: Cc1nc(NC(C)c2ccc(-c3ccccc3CN(C)C)s2)c2cc(N3CCC(N(C)C)C3)ccc2n1]

This compound was synthesized from N-[1-(5-bromothiophen-2-yl)ethyl]-6-[3-(dimethylamino)pyrrolidin-1-yl]-2-methylquinazolin-4-amine (described in example 414) by the same method as described in example 415 to give 33.4 mg (29%) of the product as a yellow solid. MS (ESIpos): m/z=515 [M+H]⁺; LC-MS [Method 4, starting with gradient water (0.05% TFA)-Acetonitrile, 5% B]: Rₜ=0.89 min. ¹H-NMR (400 MHz, DMSO-d₆): δ [ppm]=1.73 (d, 3H), 1.82-1.86 (m, 1H), 2.10 (s, 6H), 2.18-2.19 (m, 1H), 2.22 (s, 3H), 2.23 (s, 3H), 2.33 (s, 3H), 2.82-2.84 (m, 1H), 3.12-3.13 (m, 1H), 3.28 (m, 1H), 3.34 (s, 2H), 3.48-3.49 (m, 2H), 5.99-6.01 (m, 1H), 7.07 (d, 2H), 7.15 (d, 2H), 7.27-7.32 (m, 2H), 7.35 (d, 1H), 7.41-7.43 (m, 1H), 7.47 (d, 1H), 8.08 (d, 1H).

### Example 419

### N-[1-(5-bromothiophen-2-yl)ethyl]-2-methyl-6-(pyrrolidin-1-yl)quinazolin-4-amine

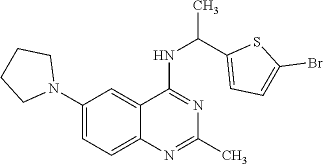 [SMILES: Cc1nc(NC(C)c2ccc(Br)s2)c2cc(N3CCCC3)ccc2n1]

### Step a

### methyl 5-fluoro-2-nitrobenzoate

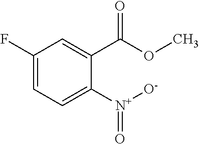

To a solution of 5-fluoro-2-nitrobenzoic acid, 10 g (54 mmol), in 100 mL of methanol was added 20 mL of sulfuric acid at 0° C. and the resulting mixture was stirred at 60° C. for 16 hours. After cooling to room temperature, aq. potassium carbonate solution was added to adjust the pH value to 8 and methanol was removed in vacuo. The resulting solution was extracted with ethyl acetate and the combined organic layers were dried over anhydrous sodium sulfate. The solvent was removed in vacuo to give 9.25 g (77%) of the product as a red oil. MS (ESIpos): m/z=200 [M+H]⁺; LC-MS [Method 4, gradient starting with water (0.05% TFA)-Acetonitrile, 5% B]: Rₜ=0.98 min.

### Step b

### methyl 2-nitro-5-(pyrrolidin-1-yl)benzoate

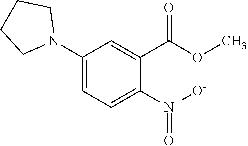 [SMILES: COC(=O)c1cc(N2CCCC2)ccc1[N+](=O)[O-]]

Methyl 5-fluoro-2-nitrobenzoate, 500 mg (2.5 mmol), pyrrolidine, 357 mg (5 mmol), and potassium carbonate, 694 mg (5 mmol), were added into 5 mL of N,N-dimethylformamide. The resulting mixture was stirred at reflux for 4 hours. After cooling to room temperature, water was added and the resulting solution was extracted with ethyl acetate. The combined organic layers were dried over anhydrous sodium sulfate and the solvent was removed in vacuo to give 550 mg (85%) of the product as a grey solid. MS (ESIpos): m/z=251 [M+H]⁺; LC-MS [Method 4, gradient starting with water (0.05% NH₄HCO₃)-Acetonitrile, 10% B]: Rₜ=1.85 min.

### Step c

### methyl 2-amino-5-(pyrrolidin-1-yl)benzoate

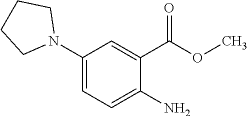 [SMILES: COC(=O)c1cc(N2CCCC2)ccc1N]

Methyl 2-nitro-5-(pyrrolidin-1-yl)benzoate, 550 mg (2.2 mmol), and palladium on carbon (10%), 233 mg (0.2 mmol), were added into 10 mL of methanol. The resulting mixture was stirred under hydrogen atmosphere (2 atm) for 3 hours at room temperature. The catalyst was removed by filtration and the filtrate was concentrated in vacuo to give 450 mg (84%) of the product as a grey solid. MS (ESIpos): m/z=221 [M+H]⁺; LC-MS [Method 4, gradient starting with water (0.05% NH₄HCO₃)-Acetonitrile, 10% B]: Rₜ=1.74 min.

### Step d

### 2-methyl-6-(pyrrolidin-1-yl)quinazolin-4(3H)-one

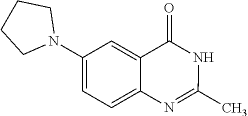 [SMILES: Cc1nc2ccc(N3CCCC3)cc2c(=O)[nH]1]

Dry HCl gas was passed (until the clear solution observed) to a solution of methyl 2-amino-5-(pyrrolidin-1-yl)benzoate, 450 mg (2 mmol), in 10 mL of acetonitrile for 3 hours at room temperature. Then the resulting mixture was stirred at reflux for 10 hours and cooled down to room temperature. The precipitated solid was collected by filtration and the filter cake was re dissolved with water. The solution was neutralized with aqueous sodium bicarbonate and the precipitated solid was collected by filtration, washed with ice cold water and dried in vacuo to give 324 mg (69%) of the product as a white solid. MS (ESIpos): m/z=230 [M+H]⁺; LC-MS [Method 4, gradient starting with water (0.05% NH₄HCO₃)-Acetonitrile, 10% B]: Rₜ=1.36 min. ¹H-NMR (300 MHz, DMSO-d₆): δ [ppm]=1.97-2.01 (m, 4H), 2.27 (s, 3H), 3.28-3.30 (m, 4H), 6.98 (s, 1H), 7.08 (d, 1H), 7.42 (d, 1H), 11.89 (br, 1H).

### Step e

### 4-chloro-2-methyl-6-(pyrrolidin-1-yl)quinazoline

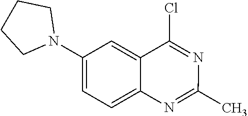 [SMILES: Cc1nc(Cl)c2cc(N3CCCC3)ccc2n1]

2-Methyl-6-(pyrrolidin-1-yl)quinazolin-4(3H)-one, 680 mg (3 mmol), was dissolved in 20 mL of phosphorus oxychloride and the resulting mixture was stirred at 110° C. for 2 hours. After cooling to room temperature, the solvent was removed in vacuo and the residue was re-dissolved with dichloromethane. Saturated sodium bicarbonate solution was added to adjust the pH value to 8. The resulting mixture was extracted with dichloromethane and the combine organic layer was concentrated in vacuo to give 720 mg (crude) of the product as a red solid. MS (ESIpos): m/z=248 [M+H]⁺; LC-MS [Method 4, gradient starting with water (0.05% NH₄HCO₃)-Acetonitrile, 10% B]: Rₜ=1.90 min.

### Step f

### N-[1-(5-bromothiophen-2-yl)ethyl]-2-methyl-6-(pyrrolidin-1-yl)quinazolin-4-amine

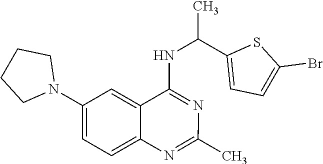 [SMILES: Cc1nc(NC(C)c2ccc(Br)s2)c2cc(N3CCCC3)ccc2n1]

4-Chloro-2-methyl-6-(pyrrolidin-1-yl)quinazoline, 200 mg (0.8 mmol), and 1-(5-bromothiophen-2-yl)ethanamine, 166 mg (0.8 mmol, described in example INT-28), were dissolved into 6 mL of propan-2-ol. The resulting mixture was stirred at 110° C. for 16 hours. After cooling to room temperature, the solvent was removed in vacuo and the residue was purified with silica gel column chromatography to give 190 mg (33%) of the product as a yellow solid. MS (ESIpos): m/z=417 [M+H]⁺; LC-MS [Method 4, gradient starting with water (0.05% TFA)-Acetonitrile, 5% B]: Rₜ=1.48 min. ¹H-NMR (400 MHz, DMSO-d₆): δ [ppm]=1.67 (d, 3H), 1.98-2.01 (m, 4H), 2.41 (s, 3H), 3.32-3.34 (m, 4H), 5.77-5.84 (m, 1H), 6.91 (s, 1H), 7.05 (d, 2H), 7.15 (d, 1H), 7.47 (d, 1H), 8.05 (d, 1H).

### Example 420

### N-(4-{[1-(5-{2-[(dimethylamino)methyl]phenyl}thiophen-2-yl)ethyl]amino}-2-methylquinazolin-6-yl)acetamide

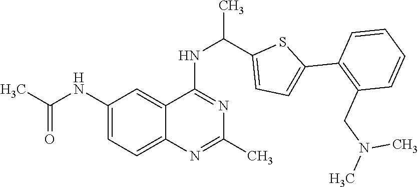 [SMILES: CC(=O)Nc1ccc2nc(C)nc(NC(C)c3ccc(-c4ccccc4CN(C)C)s3)c2c1]

N₄-(1-(5-(2-((dimethylamino)methyl)phenyl)thiophen-2-yl)ethyl)-2-methylquinazoline-4,6-diamine, 100 mg (0.24 mmol, described in example 423) and triethylamine, 73 mg (0.72 mmol), were dissolved into 3.0 mL of dichloromethane. Then acetyl chloride, 24 mg (0.31 mmol), was added dropwise to the above solution at 0° C. and the resulting mixture was stirred at room temperature for 2 hours. The solvent was removed in vacuo and the residue was purified by preparative HPLC to give 32.9 mg (29%) of the product as a white solid. MS (ESIpos): m/z=460 [M+H]⁺. LC-MS [Method 4, gradient starting with water (0.05% NH₄HCO₃)-Acetonitrile, 10% B]: Rₜ=1.87 min. ¹H-NMR (400 MHz, DMSO-d₆): δ [ppm]=1.71 (d, 3H), 2.07 (s, 3H), 2.13 (s, 6H), 2.46 (s, 3H), 3.36 (s, 2H), 5.93-5.96 (m, 1H), 7.08 (d, 1H), 7.17 (d, 1H), 7.26-7.33 (m, 2H), 7.35-7.37 (m, 1H), 7.41-7.43 (m, 1H), 7.56 (d, 1H), 7.68 (d, 1H), 8.40 (s, 1H), 8.46 (d, 1H), 10.08 (s, 1H).

### Example 421

### N-[1-(5-{2-[(dimethylamino)methyl]phenyl}thiophen-2-yl)ethyl]-2-methyl-6-nitroquinazolin-4-amine

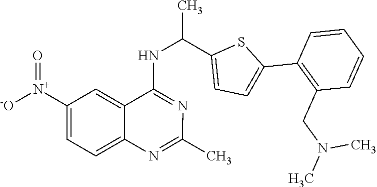

N-(1-(5-bromothiophen-2-yl)ethyl)-2-methyl-6-nitroquinazolin-4-amine, 1.90 g (4.8 mmol, described in example 452), 2-((dimethylamino)methyl)phenylboronic acid, 2.60 g (14.5 mmol), potassium carbonate, 2.67 g (19.3 mmol), and tetrakis(triphenylphosphine)palladium(0), 0.56 g (0.5 mmol), were added into 60 mL of 1,4-dioxane/water (v:v=5:1). The resulting mixture was stirred at 100° C. for 10 hours under nitrogen atmosphere. After cooling to room temperature, water was added and the mixture was extracted with ethyl acetate. The combined organic layers were dried over anhydrous sodium sulfate and the solvent was removed in vacuo. The residue was purified with silica gel column chromatography to give 1.01 g (47%) of the product as a yellow solid. MS (ESIpos): m/z=448 [M+H]⁺; LC-MS [Method 4, gradient starting with water (0.05% NH₄HCO₃)-Acetonitrile, 10% B]: Rₜ=2.29 min. ¹H-NMR (400 MHz, DMSO-d₆): δ [ppm]=1.75 (d, 3H), 2.23-2.38 (m, 6H), 2.54 (s, 3H), 3.33 (s, 2H), 5.95-6.02 (m, 1H), 7.14-7.17 (m, 2H), 7.35-7.43 (m, 3H), 7.48-7.54 (br, 1H), 7.76 (d, 1H), 8.45 (d, 1H), 9.27 (d, 1H), 9.47 (s, 1H).

### Example 422

### 6,7-dimethoxy-N-{1-[5-(4-methoxy-2-methylphenyl)thiophen-2-yl]ethyl}-2-methylquinazolin-4-amine

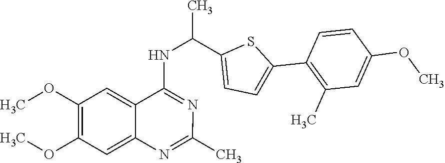

### Step a

### 2-(4-methoxy-2-methylphenyl)-4,4,5,5-tetramethyl-1,3,2-dioxaborolane

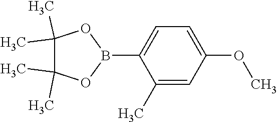

To a solution of 1-bromo-4-methoxy-2-methylbenzene, 0.50 g (2.5 mmol), in 10 mL of 1,4-dioxane were added 4,4,4′,4′,5,5,5′,5′-octamethyl-2,2′-bi(1,3,2-dioxaborolane), 1.26 g (5.0 mmol), potassium acetate, 0.98 g (10.0 mmol), and 1,1′-bis(diphenyl-phosphino)ferrocenepalladium(II) chloride, 0.18 g (0.25 mmol). The resulting mixture was stirred at 100° C. for 10 hours under nitrogen atmosphere. After cooling to room temperature, the solvent was removed in vacuo and the residue was diluted with water. The resulting mixture was extracted with ethyl acetate and the combined organic layers were dried over anhydrous sodium sulfate. The solvent was removed in vacuo and the residue was purified by silica gel column chromatography (petroleum ether: ethyl acetate=10:1) to give 0.51 g (78%) of the product as a yellow solid. MS (ESIpos): m/z=249 [M+H]⁺. LC-MS [Method 4, gradient starting with water (0.05% TFA)-Acetonitrile, 5% B]: Rₜ=1.34 min.

### Step b

### 6,7-di methoxy-N-(1-(5-(4-methoxy-2-methyl phenyl)thiophen-2-yl)ethyl)-2-methylquinazolin-4-amine

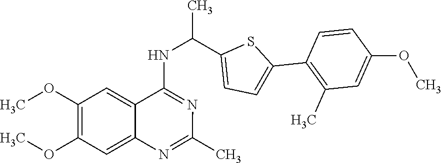

To a solution of N-(1-(5-bromothiophen-2-yl)ethyl)-6,7-dimethoxy-2-methylquinazolin-4-amine, 25 mg (0.06 mmol, described in example 209), in 3 mL of 1,4-dioxane/water (v:v=5:1) were added 2-(4-methoxy-2-methylphenyl)-4,4,5,5-tetramethyl-1,3,2-dioxaborolane, 30.0 mg (0.1 mmol), sodium carbonate, 25.6 mg (0.2 mmol) and 1,1′-bis(diphenylphosphino)ferrocenepalladium(II) chloride, 4.0 mg (6 μmol). The resulting mixture was stirred at 100° C. for 6 hours under nitrogen atmosphere. After cooling to room temperature, the solvent was removed in vacuo and the residue was diluted with water. The resulting mixture was extracted with ethyl acetate and the combined organic layers were dried over anhydrous sodium sulfate. The solvent was removed in vacuo and the residue was purified by preparative HPLC [Column: XBridge Prep C18 OBD, 5 μm, 19×150 mm; Mobile Phase A: water (0.01% NH₄HCO₃), Mobile Phase B: acetonitrile; Gradient: 10% B to 25% B in 5 min] to give 2.4 mg (9%) of the product as an off-white solid. MS (ESIpos): m/z=450 [M+H]⁺. LC-MS [Method 4, gradient starting with water (0.05% TFA)-Acetonitrile, 5% B]: Rₜ=1.49 min. ¹H-NMR (400 MHz, DMSO-d₆): δ [ppm]=1.70 (d, 3H), 2.34 (s, 3H), 2.43 (s, 3H), 3.75 (s, 3H), 3.87 (s, 6H), 5.93 5.96 (m, 1H), 6.76-6.79 (m, 1H), 6.86 (d, 1H), 6.94-6.95 (m, 1H), 7.05 (s, 2H), 7.24 (d, 1H), 7.65 (s, 1H), 8.13 (d, 1H).

### Example 423

### N₄-[1-(5-{2-[(dimethylamino)methyl]phenyl}-2-thienyl)ethyl]-2-methylquinazoline-4,6-diamine

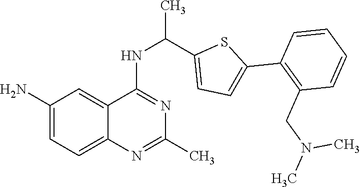 [SMILES: Cc1nc(NC(C)c2ccc(-c3ccccc3CN(C)C)s2)c2cc(N)ccc2n1]

N-(1-(5-(2-((dimethylamino)methyl)phenyl)thiophen-2-yl)ethyl)-2-methyl-6-nitroquinazolin-4-amine, 1.01 g (2.3 mmol, described in example 421), and palladium carbon (10%), 0.24 g (0.2 mmol), were added into 30 mL of methanol. The resulting mixture was stirred at room temperature for 3 hours under hydrogen atmosphere (3 atm). The catalyst was removed by filtration and the filtrate was concentrated in vacuo to give 0.71 g (54%) of the product as a light yellow solid. MS (ESIpos): m/z=418 [M+H]⁺; LC-MS [Method 4, gradient starting with water (0.05% TFA)-Acetonitrile, 5% B]: Rₜ=0.76 min. ¹H-NMR (400 MHz, DMSO-d₆): δ [ppm]=1.69 (d, 3H), 2.10 (s, 6H), 2.39 (s, 3H), 3.36 (s, 2H), 5.26 (s, 2H), 5.86-5.91 (m, 1H), 7.09 (d, 1H), 7.12 (d, 1H), 7.16-7.18 (m, 2H), 7.26-7.36 (m, 4H), 7.41-7.43 (m, 1H), 7.97 (d, 1H).

### Example 424

### N-[1-(5-bromothiophen-2-yl)ethyl]-2-methyl-6-[4-(pyridin-3-ylmethyl)piperazin-1-yl]quinazolin-4-amine

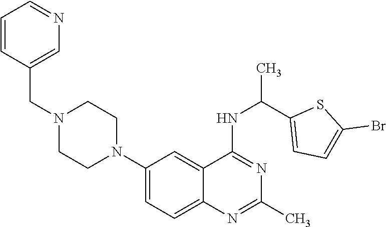 [SMILES: Cc1nc(NC(C)c2ccc(Br)s2)c2cc(N3CCN(Cc4cccnc4)CC3)ccc2n1]

### Step a

### methyl 2-nitro-5-(4-(pyridin-3-ylmethyl)piperazin-1-yl)benzoate

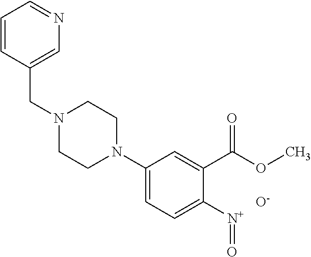

This compound was synthesized by the same method as described in example 419 (step b) to give 0.95 g (73%) of the product as a yellow solid. MS (ESIpos): m/z=357 [M+H]⁺; LC-MS [Method 4, gradient starting with water (0.05% NH₄HCO₃)-Acetonitrile, 10% B]: Rₜ=1.66 min.

### Step b

### methyl 2-amino-5-(4-(pyridin-3-ylmethyl)piperazin-1-yl)benzoate

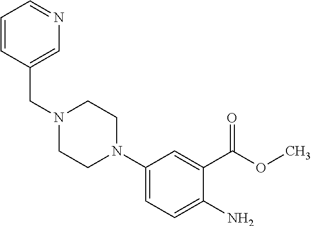 [SMILES: COC(=O)c1cc(N2CCN(Cc3cccnc3)CC2)ccc1N]

This compound was synthesized by the same method as described in example 419 (step c) to give 1 g (85%) of the product as a grey solid. MS (ESIpos): m/z=327 [M+H]⁺; LC-MS [Method 4, gradient starting with water (0.05% NH₄HCO₃)-Acetonitrile, 10% B]: Rₜ=1.42 min.

### Step c

### 2-methyl-6-(4-(pyridin-3-ylmethyl)piperazin-1-yl)quinazolin-4(3H)-one

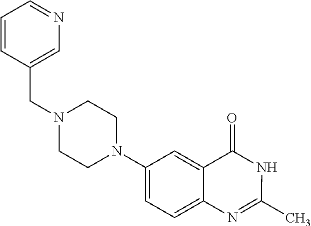 [SMILES: Cc1nc2ccc(N3CCN(Cc4cccnc4)CC3)cc2c(=O)[nH]1]

This compound was synthesized by the same method as described in example 419 (step d) to give 1.2 mg (84%) of the product as a white solid. MS (ESIpos): m/z=336 [M+H]⁺; LC-MS [Method 4, gradient starting with water (0.05% NH₄HCO₃)-Acetonitrile, 10% B]: Rₜ=1.55 min. ¹H-NMR (300 MHz, DMSO-d₆): δ [ppm]=2.29 (s, 3H), 2.54-2.56 (m, 4H), 3.21-3.24 (m, 4H), 3.58 (s, 2H), 7.36-7.38 (m, 2H), 7.44 (d, 1H), 7.51 (d, 1H), 7.75 (d, 1H), 8.48-8.53 (m, 1H), 8.54 (s, 1H).

### Step d

### 4-chloro-2-methyl-6-(4-(pyridin-3-ylmethyl)piperazin-1-yl)quinazoline

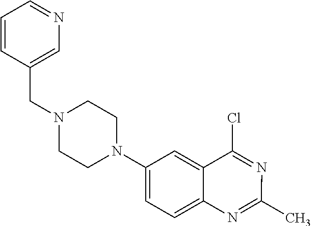 [SMILES: Cc1nc(Cl)c2cc(N3CCN(Cc4cccnc4)CC3)ccc2n1]

This compound was synthesized by the same method as described in example 419 (step e) to give 470 mg (63%) of the product as a white solid. MS (ESIpos): m/z=355 [M+H]⁺; LC-MS [Method 4, gradient starting with water (0.05% TFA)-Acetonitrile, 5% B]: Rₜ=0.56 min.

### N-[1-(5-bromothiophen-2-yl)ethyl]-2-methyl-6-[4-(pyridin-3-ylmethyl)piperazin-1-yl]quinazolin-4-amine

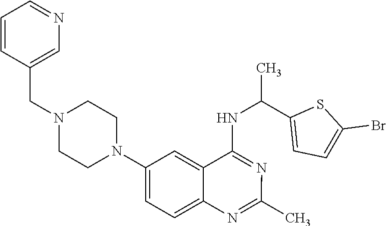 [SMILES: Cc1nc(NC(C)c2ccc(Br)s2)c2cc(N3CCN(Cc4cccnc4)CC3)ccc2n1]

This compound was synthesized by the same method as described in example 419 (step f) to give 160 mg (51%) of the product as a yellow solid. MS (ESIpos): m/z=523 [M+H]⁺; LC-MS [Method 4, gradient starting with water (0.05% TFA)-Acetonitrile, 50% B]: Rₜ=1.07 min. ¹H-NMR (400 MHz, DMSO-d₆): δ [ppm]=1.67 (d, 3H), 2.42 (s, 3H), 2.56-2.58 (m, 4H), 3.24-3.25 (m, 4H), 3.60 (s, 2H), 5.78-5.82 (m, 1H), 6.92 (d, 1H), 7.07 (d, 1H), 7.36-7.40 (m, 1H), 7.47-7.53 (m, 3H), 7.76 (d, 1H), 8.17 (d, 1H), 8.48 (d, 1H), 8.54 (s, 1H).

### Example 425

### N₄-[l-(5-bromo-2-thienyl)ethyl]-2-methyl-N₆-[2-(morpholin-4-yl)ethyl]quinazoline-4,6-diamine

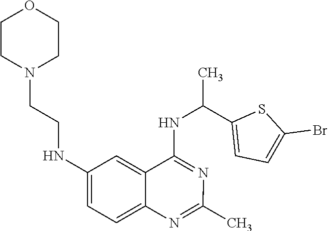 [SMILES: Cc1nc(NC(C)c2ccc(Br)s2)c2cc(NCCN3CCOCC3)ccc2n1]

### Step a

### methyl 5-(2-morpholinoethylamino)-2-nitrobenzoate

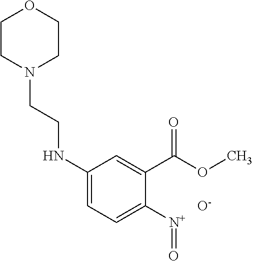

This compound was synthesized by the same method as described in example 419 (step b) to give 1.1 g (70%) of the product as a yellow solid. MS (ESIpos): m/z=310 [M+H]⁺; LC-MS [Method 4, gradient starting with water (0.05% NH₄HCO₃)-Acetonitrile, 10% B]: Rₜ=1.48 min.

### Step b

### methyl 2-amino-5-(2-morpholinoethylamino)benzoate

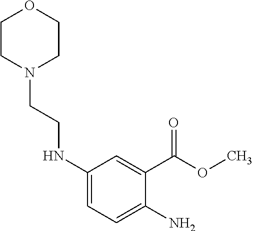 [SMILES: COC(=O)c1cc(NCCN2CCOCC2)ccc1N]

This compound was synthesized by the same method as described in example 419 (step c) to give 0.9 g (76%) of the product as a grey solid. MS (ESIpos): m/z=327 [M+H]⁺; LC-MS [Method 4, gradient starting with water (0.05% NH₄HCO₃)-Acetonitrile, 10% B]: Rₜ=1.23 min.

### Step c

### 2-methyl-6-(2-morpholinoethylamino)quinazolin-4(3H)-one

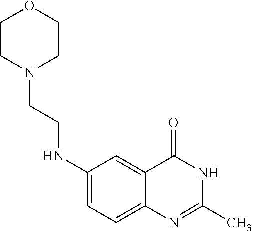 [SMILES: Cc1nc2ccc(NCCN3CCOCC3)cc2c(=O)[nH]1]

This compound was synthesized by the same method as described in example 419 (step d) to give 0.79 g (79%) of the product as a white solid. MS (ESIpos): m/z=289 [M+H]⁺; LC-MS [Method 4, gradient starting with water (0.05% NH₄HCO₃)-Acetonitrile, 10% B]: Rₜ=1.04 min. ¹H-NMR (400 MHz, DMSO-d₆): δ [ppm]=2.26 (s, 3H), 2.33-2.36 (m, 4H), 2.53-2.54 (m, 2H), 3.16-3.20 (m, 2H), 3.58-3.61 (m, 4H), 5.91-5.93 (m, 1H), 7.02 (s, 1H), 7.12 (d, 1H), 7.32 (d, 1H), 11.85 (s, 1H).

### Step d

### 4-chloro-2-methyl-N-(2-morpholinoethyl)quinazolin-6-amine

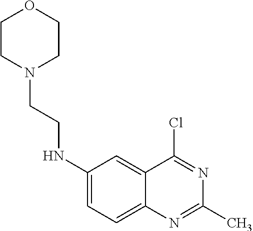 [SMILES: Cc1nc(Cl)c2cc(NCCN3CCOCC3)ccc2n1]

2-Methyl-6-(2-morpholinoethylamino)quinazolin-4(3H)-one, 300 mg (1.0 mmol), and N,N-diisopropylethylamine, 2 mL (21.5 mmol), were dissolved into 10 mL of toluene. The resulting mixture was stirred at 110° C. for 1 hour under nitrogen atmosphere. After cooling to 80° C., 3.0 mL of phosphorus oxychloride was added dropwise to the above solution and the resulting mixture was stirred at this temperature for 3 hours. After cooling to room temperature, the solvent was removed in vacuo and the residue was re-dissolved with dichloromethane. Aq. potassium carbonate solution was added to adjust pH value to 7-8 and the resulting mixture was extracted with dichloromethane. The combined organic layers were dried with anhydrous sodium sulfate and concentrated in vacuo to give 210 mg (39%) of the product as a brown solid. MS (ESIpos): m/z=307 [M+H]⁺; LC-MS [Method 4, gradient starting with water (0.05% TFA)-Acetonitrile, 5% B]: Rₜ=0.53 min.

### Step e

### N₄-[1-(5-bromo-2-thienyl)ethyl]-2-methyl-N₆-[2-(morpholin-4-yl)ethyl]quinazoline-4,6-diamine

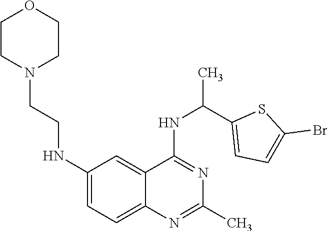 [SMILES: Cc1nc(NC(C)c2ccc(Br)s2)c2cc(NCCN3CCOCC3)ccc2n1]

This compound was synthesized from 1-(5-bromothiophen-2-yl)ethanamine (described in example INT-28) by the same method as described in example 419 (step f) to give 180 mg (57%) of the product as a yellow solid. MS (ESIpos): m/z=523 [M+H]⁺; LC-MS [Method 4, starting with water (0.05% NH₄HCO₃)-Acetonitrile, 10% B]: Rₜ=1.91 min. ¹H-NMR (400 MHz, DMSO-d₆): δ [ppm]=1.66 (d, 3H), 2.40 (s, 3H), 2.44-2.46 (m, 4H), 2.57-2.58 (m, 2H), 3.19-3.22 (m, 2H), 3.58-3.61 (m, 4H), 5.68-5.70 (m, 1H), 5.77-5.80 (m, 1H), 6.91 (d, 1H), 7.02 (s, 1H), 7.06 (d, 1H), 7.18 (d, 1H), 7.36 (d, 1H), 7.95 (d, 1H).

### Example 426

### N-[1-(5-{2-[(dimethylamino)methyl]phenyl}thiophen-2-yl)ethyl]-2-methyl-6-[4-(pyridin-3-ylmethyl)piperazin-1-yl]quinazolin-4-amine

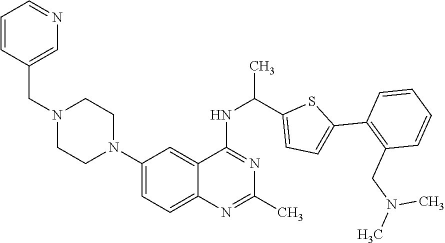 [SMILES: Cc1nc(NC(C)c2ccc(-c3ccccc3CN(C)C)s2)c2cc(N3CCN(Cc4cccnc4)CC3)ccc2n1]

This compound was synthesized from N-[1-(5-bromothiophen-2-yl)ethyl]-2-methyl-6-[4-(pyridin-3-ylmethyl)piperazin-1-yl]quinazolin-4-amine (described in example 424) by the same method as described in example 415 to give 8.2 mg (24%) of the product as a yellow solid. MS (ESIpos): m/z=579 [M+H]⁺; LC-MS [Method 4, gradient starting with water (0.05% NH₄HCO₃)-Acetonitrile, 10% B]: Rₜ=2.13 min. ¹H-NMR (400 MHz, DMSO-d₆): δ [ppm]=1.72 (d, 3H), 2.10 (s, 3H), 2.13 (s, 3H), 2.43 (s, 3H), 2.56-2.58 (m, 4H), 3.25-3.26 (m, 4H), 3.32-3.35 (m, 2H), 3.58-3.60 (m, 2H), 5.96-6.00 (m, 1H), 7.07 (d, 1H), 7.17 (d, 1H), 7.29 (d, 2H), 7.32-7.40 (m, 3H), 7.41-7.52 (m, 3H), 7.52 (d, 1H), 8.24 (d, 1H), 8.48 (d, 1H), 8.54 (d, 1H).

### Example 427

### N-{2-[(4-{[1-(5-{2-[(dimethylamino)methyl]phenyl}thiophen-2-yl)ethyl]amino}-2-methylquinazolin-6-yl)amino]ethyl}acetamide

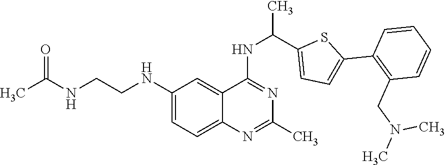 [SMILES: CC(=O)NCCNc1ccc2nc(C)nc(NC(C)c3ccc(-c4ccccc4CN(C)C)s3)c2c1]

N₄-(1-(5-(2-((dimethylamino)methyl)phenyl)thiophen-2-yl)ethyl)-2-methylquinazoline-4,6-diamine, 100 mg (0.24 mmol, described in example 423), and N-(2-chloroethyl)acetamide, 35 mg (0.29 mmol), were dissolved into 3.0 mL of toluene. The resulting mixture was stirred at 110° C. for 24 hours. After cooling to room temperature, the solvent was removed in vacuo and the residue was purified by preparative HPLC to give 11.4 mg (8%) of the product as an off-white solid. MS (ESIpos): m/z=503 [M+H]⁺. LC-MS [Method 4, gradient starting with water (0.05% TFA)-Acetonitrile, 5% B]: Rₜ=3.56 min. ¹H-NMR (300 MHz, DMSO-d₆): δ [ppm]=1.73 (d, 3H), 1.84 (s, 3H), 2.11 (s, 6H), 2.41 (s, 3H), 3.17-3.21 (m, 2H), 3.28-3.30 (m, 2H), 3.37 (s, 2H), 5.88-5.96 (m, 2H), 7.07-7.08 (m, 1H), 7.11-7.14 (m, 2H), 7.17-7.18 (m, 1H), 7.28-7.32 (m, 2H), 7.34-7.45 (m, 3H), 8.01-8.04 (m, 2H).

### Example 428

### N-[1-(5-bromo-3-chlorothiophen-2-yl)ethyl]-6,7-dimethoxy-2-methylquinazolin-4-amine

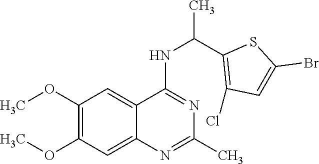 [SMILES: COc1cc2nc(C)nc(NC(C)c3sc(Br)cc3Cl)c2cc1OC]

### Step a

### 1-(5-bromo-3-chloro-2-thienyl)ethanone

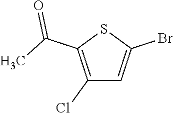

To a solution of 1-(3-chlorothiophen-2-yl)ethanone, 1.00 g (6.2 mmol), in 15 mL of chloroform was added aluminum trichloride, 2.49 g (18.7 mmol), and the resulting mixture was stirred at room temperature for 30 min. Then bromine, 1.49 g (9.3 mmol), was added to the above solution at 0° C. and the resulting mixture was stirred at reflux for 15 hours under nitrogen atmosphere. Saturated sodium sulfite solution was added and the precipitated solid was removed by filtration. The filtrate was extracted with dichloromethane and the combined organic layers were washed water, brine, dried over anhydrous sodium sulfate and concentrated in vacuo to give 1.26 g (85%) of the product as a light yellow solid. MS (ESIpos): m/z=239 [M+H]⁺. LC-MS [Method 4, Water (0.05% TFA)-Acetonitrile, 5% B]: Rₜ=1.09 min. ¹H-NMR (300 MHz, CDCl₃): δ [ppm]=2.73 (s, 3H), 7.64 (s, 1H).

### Step b

### N-[1-(5-bromo-3-chloro-2-thienyl)ethylidene]-2-methylpropane-2-sulfinamide

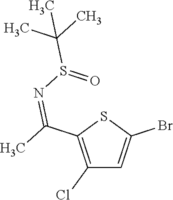 [SMILES: C/C(=N/S(=O)C(C)(C)C)c1sc(Br)cc1Cl]

To a solution of 1-(5-bromo-3-chloro-2-thienyl)ethanone, 1.26 g (5.3 mmol), in 20 mL of tetrahydrofuran was added 2-methylpropane-2-sulfinamide, 0.83 g (6.8 mmol), and titanium(IV) ethoxide, 2.40 g (10.5 mmol). The resulting mixture was stirred at 75° C. for 16 hours under nitrogen atmosphere. After cooling to room temperature, the solvent was removed in vacuo and the residue was diluted with ethyl acetate. Water was added and the precipitated solid was removed by filtration. The filtrate was extracted with ethyl acetate and combined organic layers were dried over anhydrous sodium sulfate and concentrated in vacuo to give 1.4 g (76%) of the product as a brown oil. MS (ESIpos): m/z=342 [M+H]⁺. LC-MS [Method 4, Water (0.1% HCOOH)-Acetonitrile, 10% B]: Rₜ=1.21 min.

### Step c

### N-[1-(5-bromo-3-chloro-2-thienyl)ethyl]-2-methylpropane-2-sulfinamide

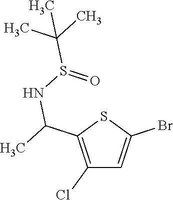 [SMILES: CC(NS(=O)C(C)(C)C)c1sc(Br)cc1Cl]

To a solution of N-[1-(5-bromo-3-chloro-2-thienyl)ethylidene]-2-methylpropane-2-sulfinamide, 1.40 g (4.1 mmol), in 15 mL of tetrahydrofuran was added sodium borohydride, 0.31 g (8.2 mmol), and the resulting mixture was stirred at room temperature for 30 min. Saturated sodium bicarbonate solution was added and the resulting mixture was stirred vigorously until gas evolution ceased. The resulting mixture was extracted with ethyl acetate and the combined organic layers were washed with water, brine, dried over anhydrous sodium sulfate and concentrated in vacuo to give 1.06 g (75%) of the product as a brown oil. MS (ESIpos): m/z=344 [M+H]⁺. LC-MS [Method 4, Water (0.1% HCOOH)-Acetonitrile, 10% B]: Rₜ=1.12 min.

### Step d

### 1-(5-bromo-3-chloro-2-thienyl)ethanamine

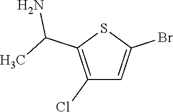

To a solution of N-[1-(5-bromo-3-chloro-2-thienyl)ethyl]-2-methylpropane-2-sulfinamide, 1.06 g (3.08 mmol), in 10 mL of tetrahydrofuran was added 3 mL of hydrochloric acid (3.0 M) and the resulting mixture was stirred at room temperature for 1 hour. Sodium hydroxide solution (6.0 M in water) was added to the above solution to adjust the pH value to 8 and the organic solvent was removed in vacuo. The resulting mixture was extracted with dichloromethane and the combined organic layers were washed water, brine, dried over anhydrous sodium sulfate and evaporated in vacuo to give 520 mg (71%) of the product as a light yellow oil. MS (ESIpos): m/z=240 [M+H]⁺. LC-MS [Method 4, Water (0.1% HCOOH)-Acetonitrile, 10% B]: Rₜ=0.54 min.

### Step e

### N-[1-(5-bromo-3-chlorothiophen-2-yl)ethyl]-6,7-dimethoxy-2-methylquinazolin-4-amine

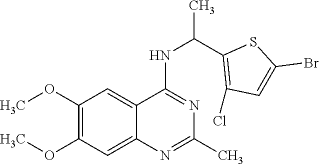 [SMILES: COc1cc2nc(C)nc(NC(C)c3sc(Br)cc3Cl)c2cc1OC]

To a solution of 1-(5-bromo-3-chloro-2-thienyl)ethanamine, 520 mg (2.2 mmol), in 8 mL of 2-propanol was added 4-chloro-6,7-dimethoxy-2-methylquinazoline, 673 mg (2.8 mmol), and the resulting mixture was stirred at 110° C. for 16 hours under nitrogen atmosphere. After cooling to room temperature, the solvent was removed in vacuo and the residue was re-dissolved with dichloromethane. The resulting solution was washed with water, brine, dried over anhydrous sodium sulfate and concentrated in vacuo. The residue was purified by silica gel column chromatography (dichloromethane:methanol=13:1) to give 280 mg (29%) of the product as a yellow solid. MS (ESIpos): m/z=442 [M+H]⁺. LC-MS [Method 4, Water (0.05% TFA)-Acetonitrile, 5% B]: Rₜ=1.33 min. ¹H-NMR (400 MHz, DMSO-d₆): δ [ppm]=1.64 (d, 3H), 2.36 (s, 3H), 3.87 (s, 3H), 3.91 (s, 3H), 5.79-5.86 (m, 1H), 7.05 (s, 1H), 7.69-7.71 (m, 2H), 8.17 (d, 1H).

### Example 429

### tert-butyl [2-(4-chloro-5-{1-[(6,7-dimethoxy-2-methylquinazolin-4-yl)amino]ethyl}thiophen-2-yl)benzyl]carbamate

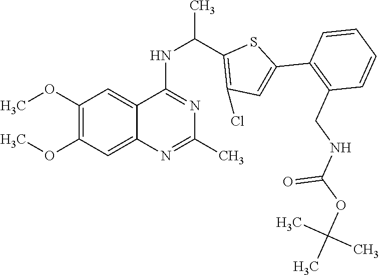 [SMILES: COc1cc2nc(C)nc(NC(C)c3sc(-c4ccccc4CNC(=O)OC(C)(C)C)cc3Cl)c2cc1OC]

### Step a

### tert-butyl (2-bromobenzyl)carbamate

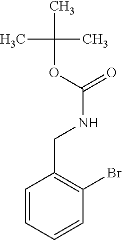 [SMILES: CC(C)(C)OC(=O)NCc1ccccc1Br]

To a solution of 2-bromophenylmethanamine, 1.50 g (8.1 mmol), in 15 mL of dichloromethane were added di-tert-butyl dicarbonate, 2.29 g (10.5 mmol), and triethylamine, 2.45 g (24.2 mmol). The resulting mixture was stirred at room temperature for 5 hours. Upon completion of the reaction, the reaction mixture was washed with water and dried over anhydrous sodium sulfate. The solvent was removed in vacuo and the residue was purified by silica gel column chromatography (petroleum ether:ethyl acetate=19:1) to give 2.08 g (79%) of the product as a light yellow oil. MS (ESIpos): m/z=286 [M+H]⁺. LC-MS [Method 4, Water (0.1% HCOOH)-Acetonitrile, 10% B]: Rₜ=1.94 min.

### Step b

tert-butyl [2-(4,4,5,5-tetramethyl-1,3,2-dioxaborolan-2-yl)benzyl]carbamate

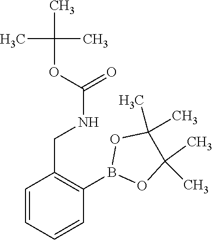 [SMILES: CC(C)(C)OC(=O)NCc1ccccc1B1OC(C)(C)C(C)(C)O1]

To a solution of tert-butyl (2-bromobenzyl)carbamate, 2.08 g (7.3 mmol), in 25 mL of 1,4-dioxane were added 4,4,4′,4′,5,5,5′,5′-octamethyl-2,2′-bi(1,3,2-dioxaborolane), 3.75 g (14.5 mmol), potassium acetate, 1.43 g (14.5 mmol), and 1,1′-bis(diphenylphosphino)ferrocenepalladiumdichloride, 0.59 g (0.7 mmol). The resulting mixture was stirred at 100° C. for 15 hours under nitrogen atmosphere. After cooling to room temperature, the solvent was removed in vacuo and the residue was diluted with water. The resulting mixture was extracted with ethyl acetate and the combined organic layers were concentrated in vacuo. The residue was purified by silica gel column chromatography (petroleum ether:ethyl acetate=7:3) to give 1.26 g (52%) of the product as a light yellow oil. MS (ESIpos): m/z=334 [M+H]⁺. LC-MS [Method 4, Water (0.1% HCOOH)-Acetonitrile, 10% B]: Rₜ=1.35 min.

### Step c

### tert-butyl [2-(4-chloro-5-{1-[(6,7-dimethoxy-2-methylquinazolin-4-yl)amino]-ethyl}thiophen-2-yl)benzyl]carbamate

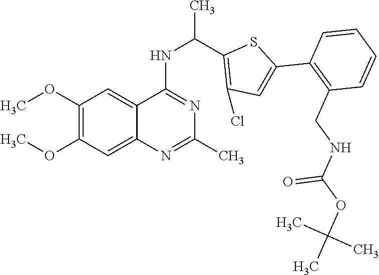 [SMILES: COc1cc2nc(C)nc(NC(C)c3sc(-c4ccccc4CNC(=O)OC(C)(C)C)cc3Cl)c2cc1OC]

To a solution of N-[1-(5-bromo-3-chloro-2-thienyl)ethyl]-6,7-dimethoxy-2-methylquinazolin-4-amine, 160 mg (0.36 mmol, described in example 428), in 15 mL of 1,4-dioxane/water (v:v=5:1) were added tert-butyl [2-(4,4,5,5-tetramethyl-1,3,2-dioxaborolan-2-yl)benzyl]carbamate, 240 mg (0.72 mmol), potassium carbonate, 150 mg (1.1 mmol) and tetrakis(triphenylphosphine)palladium(0), 42 mg (0.04 mmol). The resulting mixture was stirred at 110° C. for 16 hours under nitrogen atmosphere. After cooling to room temperature, the solvent was removed in vacuo and the residue was diluted with water. The resulting mixture was extracted with dichloromethane and the combined organic layers were concentrated in vacuo. The residue was purified by silica gel column chromatography (dichloromethane:methanol=13:1) to give 180 mg (63%) of the product as a light yellow solid. MS (ESIpos): m/z=569 [M+H]⁺. LC-MS [Method 4, Water (0.05% TFA)-Acetonitrile, 5% B]: Rₜ=1.10 min. ¹H-NMR (400 MHz, DMSO-d₆): δ [ppm]=1.37 (s, 9H), 1.71 (d, 3H), 2.41 (s, 3H), 3.89 (s, 3H), 3.93 (s, 3H), 4.01-4.07 (m, 2H), 5.87-5.91 (m, 1H), 7.06 (s, 1H), 7.16 (d, 1H), 7.27-7.44 (m, 5H), 7.77 (s, 1H), 8.55 (br, 1H).

### Example 430

### N-[1-{5-[2-(aminomethyl)phenyl]-4-chlorothiophen-2-yl}ethyl]-6,7-dimethoxy-2-methylquinazolin-4-amine

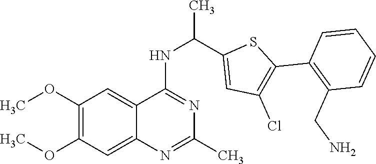 [SMILES: COc1cc2nc(C)nc(NC(C)c3cc(Cl)c(-c4ccccc4CN)s3)c2cc1OC]

### Step a

### tert-butyl-2-(3-chloro-5-(1-(6,7-dimethoxy-2-methylquinazolin-4-ylamino)ethyl)thiophen-2-yl)benzylcarbamate

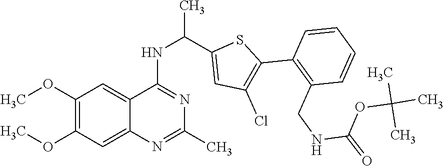 [SMILES: COc1cc2nc(C)nc(NC(C)c3cc(Cl)c(-c4ccccc4CNC(=O)OC(C)(C)C)s3)c2cc1OC]

To a solution of N-(1-(5-bromo-4-chlorothiophen-2-yl)ethyl)-6,7-dimethoxy-2-methylquinazolin-4-amine, 100 mg (0.23 mmol described in example 432), in 6.0 mL of 1,4-dioxane/water (v:v=5:1) were added 2-((tert-butoxycarbonylamino)methyl)phenylboronic acid, 90.7 mg (0.36 mmol), tetrakis(triphenylphosphine)palladium(0), 41.8 mg (36.1 μmol), and potassium carbonate, 199.8 mg (1.4 mmol). The resulting mixture was stirred at 100° C. for 10 hours under nitrogen atmosphere. After cooling to room temperature, the solvent was removed in vacuo and the residue was purified by silica gel column chromatography (dichloromethane/methanol=10:1) to give 120 mg (58%) of the product as a yellow solid. MS (ESIpos): m/z=569 [M+H]⁺; LC-MS [Method 4, Water (0.05% NH₄HCO₃)-Acetonitrile, 10% B]: Rₜ=3.39 min. ¹H-NMR (400 MHz, DMSO-d₆): δ [ppm]=1.36 (s, 9H), 1.71 (d, 3H), 2.43 (s, 3H), 3.87 (s, 3H), 3.88 (s, 3H), 4.05 (d, 2H), 5.88-5.91 (m, 1H), 7.06 (s, 1H), 7.14 (s, 1H), 7.21 (d, 1H), 7.27-7.35 (m, 3H), 7.42-7.44 (m, 1H), 7.63 (s, 1H), 8.16 (d, 1H).

### Step b

### tert-butyl-2-(3-chloro-5-(1-(6,7-dimethoxy-2-methylquinazolin-4-ylamino)ethyl)thiophen-2-yl)benzylcarbamate

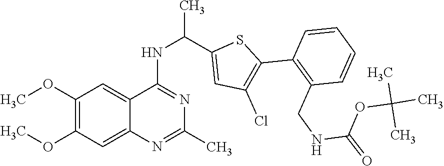 [SMILES: COc1cc2nc(C)nc(NC(C)c3cc(Cl)c(-c4ccccc4CNC(=O)OC(C)(C)C)s3)c2cc1OC]

To a solution of tert-butyl-2-(3-chloro-5-(1-(6,7-dimethoxy-2-methylquinazolin-4-ylamino)ethyl)thiophen-2-yl)benzylcarbamate, 50 mg (0.09 mmol), in 1.0 mL of 1,4-dioxane was added 1.0 mL of hydrochloric acid (4.0 M) at 0° C. and the resulting mixture was stirred at room temperature for 6 hours. Saturated sodium carbonate solution was added to adjust the pH value to 7 and the resulting mixture was extracted with ethyl acetate. The combined organic layers were washed with water, brine and concentrated in vacuo. The residue was purified by preparative HPLC [Mobile Phase A: Water (0.1% NH₄HCO₃), Mobile Phase B: Acetonitrile; Gradient: 20% B to 45% B in 7 min] to give 27.3 mg (66%) of the product as a white solid. MS (ESIpos): m/z=469 [M+H]⁺; LC-MS [Method 4, Water (0.05% NH₄HCO₃)-Acetonitrile, 10% B]: Rₜ=1.85 min. ¹H-NMR (400 MHz, DMSO-d₆): δ [ppm]=1.71 (d, 3H), 2.43 (s, 3H), 3.60 (s, 2H), 3.87 (s, 3H), 3.88 (s, 3H), 5.87-5.92 (m, 1H), 7.05 (s, 1H), 7.12 (s, 1H), 7.18 (d, 1H), 7.24 (t, 1H), 7.42 (t, 1H), 7.61-7.63 (m, 2H), 8.16 (d, 1H).

### Example 431

### N-[1-(4-chloro-5-{2-[(dimethylamino)methyl]phenyl}thiophen-2-yl)ethyl]-6,7-dimethoxy-2-methylquinazolin-4-amine

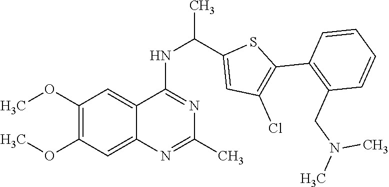 [SMILES: COc1cc2nc(C)nc(NC(C)c3cc(Cl)c(-c4ccccc4CN(C)C)s3)c2cc1OC]

To a solution of N-(1-(5-bromo-4-chlorothiophen-2-yl)ethyl)-6,7-dimethoxy-2-methylquinazolin-4-amine, 50 mg (0.11 mmol, described in example 432), in 2.4 mL of 1,4-dioxane/water (v:v=5:1) were added 2-((dimethylamino)methyl)phenylboronic acid, 20 mg (0.11 mmol), tetrakis(triphenylphosphine)palladium(0), 13 mg (11.3 μmol), and potassium carbonate, 62.4 mg (0.45 mmol). The resulting mixture was stirred at 100° C. for 10 hours under nitrogen atmosphere. After cooling to room temperature, the solvent was removed in vacuo and the residue was purified by preparative HPLC [Mobile Phase A: Water (0.1% HCOOH), Mobile Phase B: Acetonitrile; Gradient: 30% B to 70% B in 8 min] to give 20.3 mg (35%) of the product as a yellow solid. MS (ESIpos): m/z=497 [M+H]⁺; LC-MS [Method 4, Water (0.05% NH₄HCO₃)-Acetonitrile, 10% B]: Rₜ=2.15 min. ¹H-NMR (300 MHz, DMSO-d₆): δ [ppm]=1.73 (d, 3H), 2.02 (s, 6H), 2.43 (s, 3H), 3.26 (s, 2H), 3.88 (s, 3H), 3.89 (s, 3H), 5.84-5.92 (m, 1H), 7.06 (s, 1H), 7.11 (s, 1H), 7.22 (d, 1H), 7.30 (t, 1H), 7.41 (t, 1H), 7.53 (d, 1H), 7.64 (s, 1H), 8.15 (d, 1H).

### Example 432

### N-[1-(5-bromo-4-chlorothiophen-2-yl)ethyl]-6,7-dimethoxy-2-methylquinazolin-4-amine

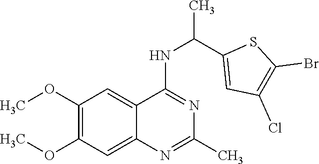 [SMILES: COc1cc2nc(C)nc(NC(C)c3cc(Cl)c(Br)s3)c2cc1OC]

### Step a

### 1-(5-bromo-4-chlorothiophen-2-yl)ethanone

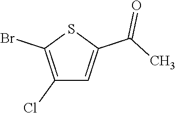

To a solution of 1-(4-chlorothiophen-2-yl)ethanone, 1.00 g (6.2 mmol), in 15 mL of chloroform was added aluminum trichloride, 2.49 g (18.7 mmol). Then bromine, 1.49 g (9.3 mmol), was added dropwise to the above solution at 0° C. and the reaction mixture was stirred at 60° C. for 1 hour. After cooling to room temperature, saturated sodium sulfite solution was added and the resulting mixture was extracted with dichloromethane. The combined organic layers were dried over anhydrous sodium sulfate and concentrated in vacuo to give 1.28 g (69%) of the product as a light yellow oil. MS (ESIpos): m/z=238 [M+H]⁺; LC-MS [Method 4, Water (0.05% TFA)-Acetonitrile, 5% B]: Rₜ=1.15 min.

### Step b

### N-(1-(5-bromo-4-chlorothiophen-2-yl)ethylidene)-2-methylpropane-2-sulfinamide

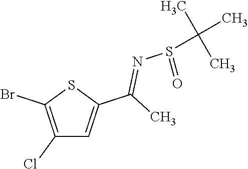 [SMILES: C/C(=N\S(=O)C(C)(C)C)c1cc(Cl)c(Br)s1]

To a solution of 1-(5-bromo-4-chlorothiophen-2-yl)ethanone, 600 mg (2.5 mmol), in 10 mL of tetrahydrofuran were added 2-methylpropane-2-sulfinamide, 395 mg (3.3 mmol), and titanium(IV) ethoxide, 1143 mg (5.0 mmol). The resulting mixture was stirred at 75° C. for 15 hours under nitrogen atmosphere. After cooling to room temperature, the solvent was removed in vacuo and the residue was re-dissolved with ethyl acetate. Water was added and the precipitated solid was removed by filtration. The filtrate was extracted with ethyl acetate and the combined organic layers were dried over anhydrous sodium sulfate. The solvent was removed in vacuo to give 0.72 g (83%) of the product as a yellow oil. MS (ESIpos): m/z=342 [M+H]⁺; LC-MS [Method 4, Water (0.05% TFA)-Acetonitrile, 5% B]: Rₜ=1.28 min.

### Step c

### N-(−1-(5-bromo-4-chlorothiophen-2-yl)ethyl)-2-methylpropane-2-sulfinamide

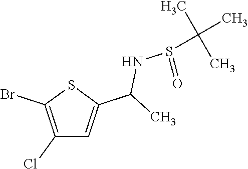 [SMILES: CC(NS(=O)C(C)(C)C)c1cc(Cl)c(Br)s1]

To a solution of N-(1-(5-bromo-4-chlorothiophen-2-yl)ethylidene)-2-methylpropane-2-sulfinamide, 720 mg (2.08 mmol), in 10 mL of tetrahydrofuran was added sodium borohydride, 199 mg (5.3 mmol), and the resulting mixture was stirred at this temperature for 5 hours. Saturated sodium bicarbonate solution was added and the resulting mixture was stirred vigorously until gas evolution ceased. The resulting mixture was extracted with ethyl acetate and the combined organic layers were dried over anhydrous sodium sulfate. The solvent was removed in vacuo to give 740 mg (82%) of the product as a yellow oil. MS (ESIpos): m/z=344 [M+H]⁺; LC-MS [Method 4, Water (0.05% NH₄HCO₃)-Acetonitrile, 10% B]: Rₜ=1.17 min.

### Step d

### 1-(5-bromo-4-chlorothiophen-2-yl)ethanamine

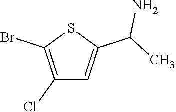

To a solution of N-(1-(5-bromo-4-chlorothiophen-2-yl)ethyl)-2-methylpropane-2-sulfinamide, 200 mg (0.6 mmol), in 5 mL of tetrahydrofuran was added 0.5 mL of hydrochloric acid (3.0 M) and the resulting mixture was stirred at room temperature for 1 hour. Sodium hydroxide solution (6M) was added to adjust the pH value to 7-8 and the resulting mixture was concentrated under vacuum. The residue was purified by silica gel column chromatography (dichloromethane:methanol=8:1) to give 100 mg (52%) of the product as a yellow oil. MS (ESIpos): m/z=240 [M+H]⁺; LC-MS [Method 4, Water (0.05% TFA)-Acetonitrile, 5% B]: Rₜ=0.72 min.

### Step e

### N-[1-(5-bromo-4-chlorothiophen-2-yl)ethyl]-6,7-dimethoxy-2-methylquinazolin-4-amine

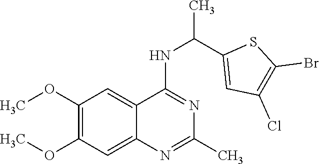 [SMILES: COc1cc2nc(C)nc(NC(C)c3cc(Cl)c(Br)s3)c2cc1OC]

To a solution of 4-chloro-6,7-dimethoxy-2-methylquinazoline, 100 mg (0.4 mmol), in 2.0 mL of 2-propanol was added 1-(5-bromo-4-chlorothiophen-2-yl)ethanamine, 100.8 mg (0.4 mmol) and the resulting mixture was stirred at 110° C. for 16 hours under nitrogen atmosphere. After cooling to room temperature, the solvent was removed in vacuo and the residue was purified by silica gel column chromatography (dichloromethane/methanol=13:1) to give 100 mg (53%) of the product as a yellow solid. MS (ESIpos): m/z=442 [M+H]⁺; LC-MS [Method 4, Water (0.05% NH₄HCO₃)-Acetonitrile, 10% B]: Rₜ=2.11 min. ¹H-NMR (400 MHz, DMSO-d₆): δ [ppm]=1.68 (d, 3H), 2.44 (s, 3H), 3.88 (s, 6H), 5.74-5.77 (m, 1H), 7.07 (s, 1H), 7.12 (s, 1H), 7.61 (s, 1H), 8.11 (d, 1H).

### Example 433

### N₄-[1-(5-{2-[(dimethylamino)methyl]phenyl}-2-thienyl)ethyl]-2-methyl-N₆-[2-(morpholin-4-yl)ethyl]quinazoline-4,6-diamine

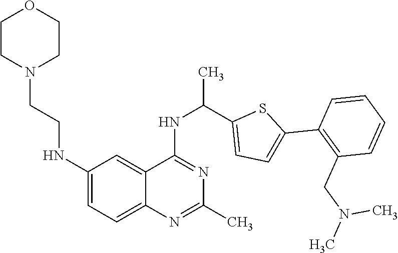 [SMILES: Cc1nc(NC(C)c2ccc(-c3ccccc3CN(C)C)s2)c2cc(NCCN3CCOCC3)ccc2n1]

This compound was synthesized by the same method as described in example 415 to give 14.4 mg (40%) of the product as a yellow solid. MS (ESIpos): m/z=531 [M+H]⁺; LC-MS [Method 4, gradient starting with water (0.05% NH₄HCO₃)-Acetonitrile, 10% B]: Rₜ=2.03 min. ¹H-NMR (400 MHz, DMSO-d₆): δ [ppm]=1.71 (d, 3H), 2.10 (s, 6H), 2.33 (s, 3H), 2.40-2.44 (m, 4H), 2.55-2.59 (m, 2H), 3.20-3.23 (m, 2H), 3.25-3.36 (m, 2H), 3.58-3.59 (m, 4H), 5.66-5.69 (m, 1H), 5.94-5.98 (m, 1H), 7.07 (d, 2H), 7.17 (d, 2H), 7.26-7.36 (m, 4H), 7.42 (d, 1H), 8.02 (d, 1H).

### Example 434

### 4-(4-{[1-(5-bromothiophen-2-yl)ethyl]amino}-2-methylquinazolin-6-yl)-1-methylpiperazin-2-one

### Step a

### methyl 5-(4-methyl-3-oxopiperazin-1-yl)-2-nitrobenzoate

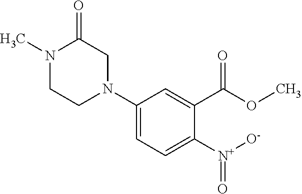

This compound was synthesized by the same method as described in example 419 (step b) to give 0.9 g (71%) of the product as a yellow solid. MS (ESIpos): m/z=294 [M+H]⁺; LC-MS [Method 4, gradient starting with water (0.1% HCOOH)-Acetonitrile, 5% B)]: Rₜ=0.99 min.

### Step b

### methyl 2-amino-5-(4-methyl-3-oxopiperazin-1-yl)benzoate

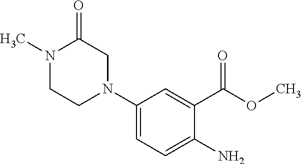

This compound was synthesized by the same method as described in example 419 (step c) to give 0.76 g (91%) of the product as a grey solid. MS (ESIpos): m/z=264 [M+H]⁺; LC-MS [Method 4, gradient starting with water (0.05% NH₄HCO₃)-Acetonitrile, 10% B]: Rₜ=1.16 min.

### Step c

### 2-methyl-6-(4-methyl-3-oxopiperazin-1-yl)quinazolin-4(3H)-one

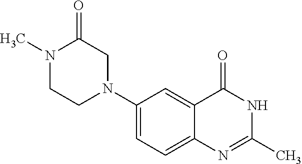

This compound was synthesized by the same method as described in example 419 (step d) to give 470 mg (60%) of the product as a light yellow solid. MS (ESIpos): m/z=273 [M+H]⁺; LC-MS [Method 4, gradient starting with water (0.05% TFA)-Acetonitrile, 5% B]: Rₜ=0.62 min. ¹H-NMR (300 MHz, DMSO-d₆): δ [ppm]=2.30 (s, 3H), 2.91 (s, 3H), 3.43-3.50 (m, 2H), 3.56-3.60 (m, 2H), 3.84 (s, 2H), 7.35 (s, 1H), 7.50 (d, 2H), 12.04 (s, 1H).

### Step d

### 4-(4-chloro-2-methylquinazolin-6-yl)-1-methyl piperazin-2-one

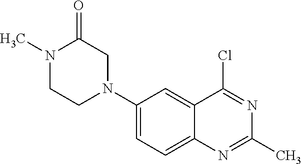

This compound was synthesized by the same method as described in example 425 (step d) to give 100 mg (76%) of the product as a brown solid. MS (ESIpos): m/z=291 [M+H]⁺; LC-MS [Method 4, gradient starting with water (0.05% TFA)-Acetonitrile, 5% B]: Rₜ=1.03 min.

### Step e

### 4-(4-(1-(5-bromothiophen-2-yl)ethylamino)-2-methylquinazolin-6-yl)-1-methylpiperazin-2-one

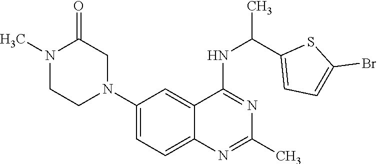 [SMILES: Cc1nc(NC(C)c2ccc(Br)s2)c2cc(N3CCN(C)C(=O)C3)ccc2n1]

This compound was synthesized from 1-(5-bromothiophen-2-yl)ethanamine (described in example INT-28) by the same method as described in example 419 (step f) to give 180 mg (57%) of the product as a yellow solid. MS (ESIpos): m/z=461 [M+H]⁺; LC-MS [Method 4, gradient starting with water (5 mM NH₄HCO₃)-Acetonitrile, 10% B)]: Rₜ=1.85 min. ¹H-NMR (400 MHz, DMSO-d₆): δ [ppm]=1.67 (d, 3H), 2.33 (s, 3H), 2.96 (s, 3H), 3.47-3.49 (m, 2H), 3.58 3.61 (m, 2H), 3.87-3.89 (m, 2H), 5.80-5.83 (m, 1H), 6.92 (d, 1H), 7.07 (d, 1H), 7.43 (s, 1H), 7.52 (d, 1H), 7.58 (d, 1H), 8.27 (d, 1H).

### Example 435

### 4-(4-{[1-(5-{2-[(dimethylamino)methyl]phenyl}thiophen-2-yl)ethyl]amino}-2-methylquinazolin-6-yl)-1-methylpiperazin-2-one

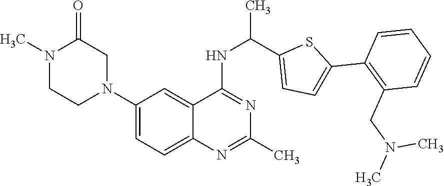 [SMILES: Cc1nc(NC(C)c2ccc(-c3ccccc3CN(C)C)s2)c2cc(N3CCN(C)C(=O)C3)ccc2n1]

This compound was synthesized from 4-(4-(1-(5-bromothiophen-2-yl)ethylamino)-2-methylquinazolin-6-yl)-1-methylpiperazin-2-one (described in example 434) by the same method as described in example 415 to give 3.3 mg (5%) of the product as a yellow solid. MS (ESIpos): m/z=516 [M+H]⁺; LC-MS [Method 4, gradient starting with water (0.05% NH₄HCO₃)-Acetonitrile, 10% B]: Rₜ=1.95 min. ¹H-NMR (400 MHz, DMSO-d₆): δ [ppm]=1.74 (d, 3H), 2.08 (s, 3H), 2.12 (s, 3H), 2.44 (s, 3H), 2.67 (s, 3H), 3.35-3.37 (m, 2H), 3.47-3.49 (m, 2H), 3.58-3.60 (m, 2H), 3.88-3.90 (m, 2H), 5.98-6.02 (m, 1H), 7.09 (d, 1H), 7.18 (d, 1H), 7.31 (d, 2H), 7.36-7.38 (m, 1H), 7.43 (d, 1H), 7.50-7.51 (m, 1H), 7.52 (s, 1H), 7.59 (d, 1H), 8.38 (d, 1H).

### Example 436

### methyl 2-[2-(5-{1-[(6,7-dimethoxy-2-methylquinazolin-4-yl)amino]ethyl}thiophen-2-yl)phenyl]-2-methylpropanoate

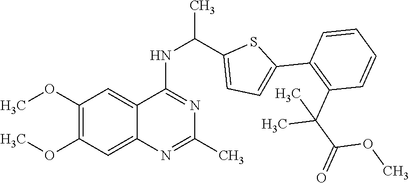

### Step a

### Methyl 2-(2-bromophenyl)-2-methylpropanoate

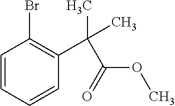

To a solution of methyl 2-(2-bromophenyl)acetate, 5.00 g (21.8 mmol), in 100 mL of tetrahydrofuran were added 18-crown-6, 1.44 g (5.5 mmol), iodomethane, 7.35 g (65.5 mmol), and potassium tert-butoxide, 15.49 g (109.1 mmol). The resulting mixture was stirred at this temperature for 26 hours. The solvent was removed in vacuo and the residue was diluted with ethyl acetate. The resulting solution was washed with water, brine, dried over anhydrous sodium sulfate. The solvent was removed in vacuo and the residue was purified by silica gel column chromatography (petroleum ether:ethyl acetate=6:1) to give 2.73 g (49%) of the product as a yellow oil. MS (ESIpos): m/z=257 [M+H]⁺. LC-MS [Method 4, Water (0.1% HCOOH)-Acetonitrile, 10% B]: Rₜ=1.14 min.

### Step b

### methyl 2-[2-(5-acetyl-2-thienylphenyl]-2-methylpropanoate

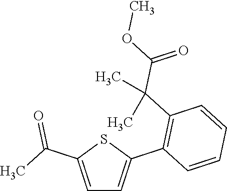 [SMILES: COC(=O)C(C)(C)c1ccccc1-c1ccc(C(C)=O)s1]

To a solution of methyl 2-(2-bromophenyl)-2-methylpropanoate, 2.50 g (7.4 mmol), in 24 mL of 1,4-dioxane/water (v:v=5:1) were added 5-acetylthiophen-2-ylboronic acid, 1.26 g (7.4 mmol), 1,1′-bis(diphenylphosphino)ferrocenepalladiumdichloride CH₂Cl₂, 0.60 g (0.7 mmol), and sodium carbonate, 1.57 g (14.8 mmol). The resulting mixture was stirred at 100° C. for 22 hours under nitrogen atmosphere. After cooling to room temperature, the solvent was removed in vacuo and the residue was diluted with ethyl acetate. The resulting solution was washed water and concentrated in vacuo. The residue was purified by silica gel column chromatography (petroleum ether:ethyl acetate=7:1) to give 400 mg (17%) of the product as a light yellow solid. MS (ESIpos): m/z=303 [M+H]⁺. LC-MS [Method 4, Water (0.1% HCOOH)-Acetonitrile, 10% B]: Rₜ=1.15 min.

### Step c

### methyl 2-(2-{5-[N-(tert-butylsulfinyl)ethanimidoyl]-2-thienyl}phenyl)-2-methylpropanoate

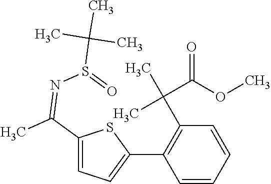 [SMILES: COC(=O)C(C)(C)c1ccccc1-c1ccc(/C(C)=N\S(=O)C(C)(C)C)s1]

To a solution of methyl 2-[2-(5-acetyl-2-thienyl)phenyl]-2-methylpropanoate, 400 mg (1.33 mmol), in 10 mL of tetrahydrofuran were added 2-methylpropane-2-sulfinamide, 211 mg (1.73 mmol), and titanium(IV) ethoxide, 610 mg (2.7 mmol). The resulting mixture was stirred at 75° C. for 16 hours under nitrogen atmosphere. After cooling to room temperature, the solvent was removed in vacuo and the residue was diluted with ethyl acetate. Water was added and the solid was removed by filtration. The filtrate was extracted with ethyl acetate and the combined organic layers were dried over anhydrous sodium sulfate. The solvent was removed in vacuo to give 500 mg (93%) of the product as a brown oil. MS (ESIpos): m/z=406 [M+H]⁺. LC-MS [Method 4, Water (0.1% HCOOH)-Acetonitrile, 10% B]: Rₜ=2.25 min.

### Step d

### methyl (2-{5-[1-{[tert-butylsulfinyl]amino}ethyl]-2-thienyl}phenyl)-2-methylpropanoate

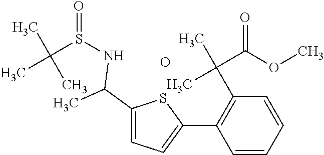

To a solution of methyl 2-(2-{5-[N-(tert-butylsulfinyl)ethanimidoyl]-2-thienyl}phenyl)-2-methylpropanoate, 500 mg (1.5 mmol), in 10 mL of tetrahydrofuran was added sodium borohydride, 115 mg (3.0 mmol), and the resulting mixture was stirred at room temperature for 5 hours. Saturated sodium bicarbonate was added and the resulting mixture was stirred vigorously until gas evolution ceased. The resulting mixture was extracted with ethyl acetate and the combined organic layers were washed with water, brine, dried over anhydrous sodium sulfate and concentrated in vacuo to give 455 mg (74%) of the product as a brown oil. MS (ESIpos): m/z=408 [M+H]⁺. LC-MS [Method 4, Water (0.05% NH₄HCO₃)-Acetonitrile, 10% B]: Rₜ=2.03 min.

### Step e

### methyl 2-{2-[5-(1-aminoethyl)-2-thienyl]phenyl}-2-methylpropanoate

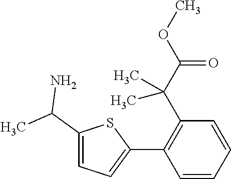 [SMILES: COC(=O)C(C)(C)c1ccccc1-c1ccc(C(C)N)s1]

To a solution of methyl (2-{5-[1-{tert-butylsulfinyl]amino}ethyl]-2-thienyl}phenyl)-2-methylpropanoate, 455 mg (1.11 mmol), in 10 mL of tetrahydrofuran was added 2.0 mL of hydrochloric acid (3.0 M) at 0° C. and the resulting mixture was stirred at room temperature for 1 hour. Sodium hydroxide solution (6.0 M) was added to adjust the pH value to 8 and the organic solvent was removed in vacuo. The resulting mixture was extracted with dichloromethane and the combined organic layers were washed with water, brine, dried over anhydrous sodium sulfate and evaporated to dryness to give 240 mg (71%) of the product as a light yellow solid. MS (ESIpos): m/z=304 [M+H]⁺. LC-MS [Method 4, Water (0.1% HCOOH)-Acetonitrile, 10% B]: Rₜ=0.77 min.

### Step f

### methyl 2-[2-(5-{1-[(6,7-dimethoxy-2-methylquinazolin-4-yl)amino]ethyl}thiophen-2-yl)phenyl]-2-methylpropanoate

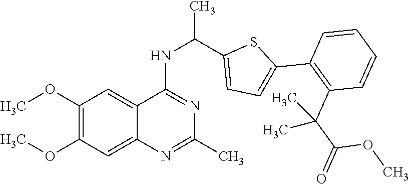

To a solution of methyl 2-{2-[5-(1-aminoethyl)-2-thienyl]phenyl}-2-methylpropanoate, 240 mg (0.8 mmol), in 6.0 mL of 2-propanol was added 4-chloro-6,7-dimethoxy-2-methylquinazoline, 189 mg (0.8 mmol), and the resulting mixture was stirred at 110° C. for 16 hours under nitrogen atmosphere. After cooling to room temperature, the solvent was removed in vacuo and the residue was diluted with dichloromethane. The resulting solution was washed with water, brine, dried over anhydrous sodium sulfate and concentrated in vacuo. The residue was purified by silica gel column chromatography (dichloromethane:methanol=13:1) to give 300 mg (60%) of the product as a yellow solid. MS (ESIpos): m/z=506 [M+H]⁺. LC-MS [Water Method 4, (0.05% TFA)-Acetonitrile, 5% B]: Rₜ=1.45 min. ¹H-NMR (400 MHz, DMSO-d₆): δ [ppm]=1.44 (s, 6H), 1.71 (d, 3H), 2.42 (s, 3H), 3.26 (s, 3H), 3.87 (s, 6H), 5.90-5.96 (m, 1H), 6.70 (s, 1H), 6.95 (s, 1H), 7.04 (s, 1H), 7.13 (d, 1H), 7.25 (t, 1H), 7.38 (t, 1H), 7.51 (d, 1H), 7.65 (s, 1H), 8.12 (d, 1H).

### Example 437

### N-[1-{5-[2-(aminomethyl)phenyl]-3-chlorothiophen-2-yl}ethyl]-6,7-dimethoxy-2-methylquinazolin-4-amine

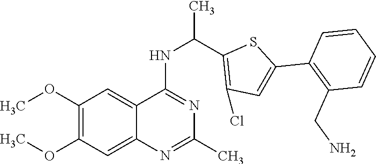 [SMILES: COc1cc2nc(C)nc(NC(C)c3sc(-c4ccccc4CN)cc3Cl)c2cc1OC]

To a solution of tert-butyl [2-(4-chloro-5-{1-[(6,7-dimethoxy-2-methylquinazolin-4-yl)amino]ethyl}-2-thienyl)benzyl]carbamate, 80 mg (0.14 mmol, described in example 429), in 3.0 mL of 1,4-dioxane was added 3.0 mL of hydrochloric acid (4.0 M), and the resulting mixture was stirred at room temperature for 6 hours. Saturated sodium carbonate solution was added to adjust the pH value to 7 and the resulting mixture was extracted with ethyl acetate. The combined organic layers were washed with water and concentrated in vacuo. The residue was purified by preparative HPLC [Mobile Phase A: Water (0.1% NH₄HCO₃), Mobile Phase B: Acetonitrile; Gradient: 25% B to 50% B in 7 min] to give 19.6 mg (28%) of the product as an off-white solid. MS (ESIpos): m/z=469 [M+H]⁺. LC-MS [Method 4, Water (0.05% NH₄HCO₃)-Acetonitrile, 10% B]: Rₜ=1.82 min. ¹H-NMR (400 MHz, DMSO-d₆): δ [ppm]=1.69 (d, 3H), 2.50 (s, 3H), 3.66-3.74 (m, 2H), 3.92 (s, 3H), 3.95 (s, 3H), 5.84-5.91 (m, 1H), 7.05 (s, 1H), 7.19 (d, 1H), 7.33-7.37 (m, 1H), 7.41-7.46 (m, 2H), 7.60 (d, 1H), 7.72 (s, 1H), 8.19 (d, 1H), 8.25 (s, 1H).

### Example 438

### N-(4-{[1-(5-{2-[(dimethylamino)methyl]phenyl}thiophen-2-yl)ethyl]amino}-2-methylquinazolin-6-yl)methanesulfonamide

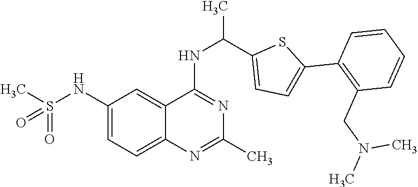 [SMILES: Cc1nc(NC(C)c2ccc(-c3ccccc3CN(C)C)s2)c2cc(NS(C)(=O)=O)ccc2n1]

### Step a

### N-(4-(1-(5-(2-((dimethylamino)methyl)phenyl)thiophen-2-yl)ethylamino)-2-methylquinazolin-6-yl)-N-(methylsulfonyl)methanesulfonamide

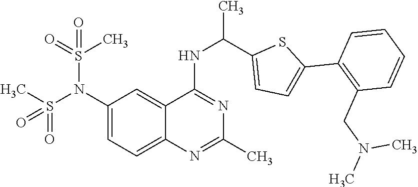 [SMILES: Cc1nc(NC(C)c2ccc(-c3ccccc3CN(C)C)s2)c2cc(N(S(C)(=O)=O)S(C)(=O)=O)ccc2n1]

N₄-(1-(5-(2-((dimethylamino)methyl)phenyl)thiophen-2-yl)ethyl)-2-methylquinazoline-4,6-diamine, 100 mg (0.24 mmol, described in example 423), and triethylamine, 121 mg (1.2 mmol), were dissolved into 3.0 mL of dichloromethane. Then methanesulfonyl chloride, 82 mg (0.72 mmol), was added dropwise to the above solution at 0° C. and the resulting mixture was stirred at room temperature for 2 hours. The solvent was removed in vacuo to give 140 mg (crude) of the product as a yellow solid and it was used directly for next step without further purification. MS (ESIpos): m/z=574 [M+H]⁺. LC-MS [Method 4, gradient starting with water (0.05% TFA)-Acetonitrile, 5% B]: Rₜ=0.78 min.

### Step b

### N-(4-{[1-(5-{2-[(dimethylamino)methyl]phenyl}thiophen-2-yl)ethyl]amino}-2-methylquinazolin-6-yl)methanesulfonamide

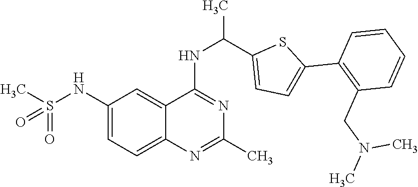 [SMILES: Cc1nc(NC(C)c2ccc(-c3ccccc3CN(C)C)s2)c2cc(NS(C)(=O)=O)ccc2n1]

To a solution of N-(4-(1-(5-(2-((dimethylamino)methyl)phenyl)thiophen-2-yl)ethylamino)-2-methylquinazolin-6-yl)-N-(methylsulfonyl)methanesulfonamide, 140 mg (0.24 mmol), in 2.0 mL of tetrahydrofuran was added sodium hydroxide solution, 30 mg (0.73 mmol) (in 2.0 mL of water). The resulting mixture was stirred at room temperature for 3 hours. The solvent was removed in vacuo and the residue was purified by preparative HPLC to give 36.0 mg (29%) of the product as a light yellow solid. MS (ESIpos): m/z=496 [M+H]⁺. LC-MS [Method 4, gradient starting with water (0.05% NH₄HCO₃)-Acetonitrile, 10% B]: Rₜ=1.86 min. ¹H-NMR (400 MHz, DMSO-d₆): δ [ppm]=1.73 (d, 3H), 2.10 (s, 6H), 2.47 (s, 3H), 3.05 (s, 3H), 3.36 (s, 2H), 5.95 5.98 (m, 1H), 7.09 (d, 1H), 7.18 (d, 1H), 7.27-7.43 (m, 4H), 7.54 (d, 1H), 7.61 (d, 1H), 8.01 (s, 1H), 8.57 (d, 1H), 9.78 (br, 1H).

### Example 439

### N-[1-(5-{2-[(dimethylamino)methyl]-4-methoxyphenyl}thiophen-2-yl)ethyl]-6,7-dimethoxy-2-methylquinazolin-4-amine

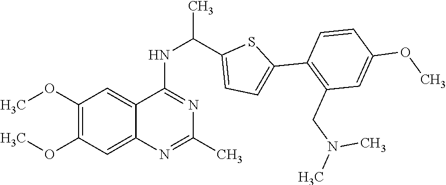

### Step a

### 1-(5-(2-((dimethylamino)methyl)-4-methoxyphenyl)thiophen-2-yl)ethanone

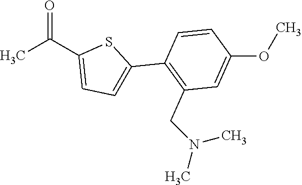 [SMILES: COc1ccc(-c2ccc(C(C)=O)s2)c(CN(C)C)c1]

(2-Bromo-5-methoxyphenyl)-N, N-dimethylmethanamine, 200 mg (0.8 mmol), 5-acetylthiophen-2-ylboronic acid, 279 mg (1.6 mmol), sodium carbonate, 347 mg (3.3 mmol) and 1,1′-bis(diphenylphosphino)ferrocenepalladium(II) chloride, 60 mg (0.08 mmol), were added into 6.0 mL of 1,4-dioxane/H₂O (v:v=5:1). The resulting mixture was stirred at 100° C. for 48 hours under nitrogen atmosphere. After cooling to room temperature, water was added and the resulting mixture was extracted with ethyl acetate. The combined organic layers were dried over anhydrous sodium sulfate and the solvent was removed in vacuo. The residue was purified with silica gel column chromatography to give 60 mg (22%) of the product as a brown solid. MS (ESIpos): m/z=290 [M+H]⁺. LC-MS [Method 4, gradient starting with water (0.05% TFA)-Acetonitrile, 5% B]: Rₜ=0.77 min.

### Step b

### N-(1-(5-(2-((dimethylamino)methyl)-4-methoxyphenyl)thiophen-2-yl)ethylidene)-2-methyl propane-2-sulfinamide

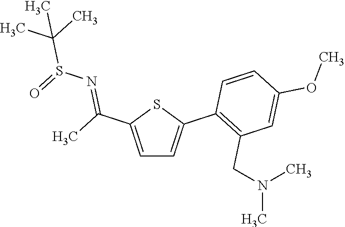 [SMILES: COc1ccc(-c2ccc(/C(C)=N/S(=O)C(C)(C)C)s2)c(CN(C)C)c1]

1-(5-(2-((Dimethylamino)methyl)-4-methoxyphenyl)thiophen-2-yl)ethanone, 80 mg (0.28 mmol), was dissolved into 3.0 mL of tetrahydrofuran. 2-methylpropane-2-sulfinamide, 44 mg (0.36 mmol), and titanium(IV) ethoxide, 252 mg (1.11 mmol), were added successively. The resulting mixture was stirred at 70° C. for 36 hours under nitrogen atmosphere. Sodium chloride solution was added and the precipitated solid was removed by filtration. The filtrate was extracted with ethyl acetate and the combined organic layers were dried over anhydrous sodium sulfate. The solvent was removed in vacuo and the residue was purified with silica gel column chromatography to give 50 mg (44%) of the product as a brown solid. MS (ESIpos): m/z=393 [M+H]⁺. LC-MS [Method 4, gradient starting with water (0.05% TFA)-Acetonitrile, 5% B]: Rₜ=0.86 min.

### Step c

### N-(1-(5-(2-((dimethylamino)methyl)-4-methoxyphenyl)thiophen-2-yl)ethyl)-2-methyl propane-2-sulfinamide

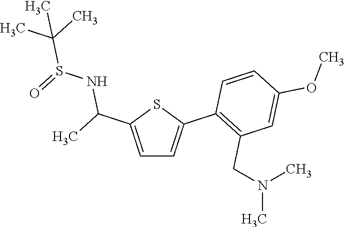 [SMILES: COc1ccc(-c2ccc(C(C)NS(=O)C(C)(C)C)s2)c(CN(C)C)c1]

N-(1-(5-(2-((dimethylamino)methyl)-4-methoxyphenyl)thiophen-2-yl)ethylidene)-2-methylpropane-2-sulfinamide, 50.0 mg (0.13 mmol), was dissolved into 3.0 mL of tetrahydrofuran. Sodium borohydride, 9.6 mg (0.26 mmol), was added successively to the above solution and the resulting mixture was stirred at room temperature for 2 hours. Water was added and the resulting solution was extracted with ethyl acetate. The combined organic layers were dried over anhydrous sodium sulfate. The solvent was removed in vacuo to give 50.0 mg (crude) of the product as a yellow solid and it was used directly for next step without further purification. MS (ESIpos): m/z=395 [M+H]⁺. LC-MS [Method 4, gradient starting with water (0.05% TFA)-Acetonitrile, 5% B]: Rₜ=0.88 min.

### Step d

### (1-(5-(2-((dimethylamino)methyl)-4-methoxyphenyl)thiophen-2-yl)ethanamine

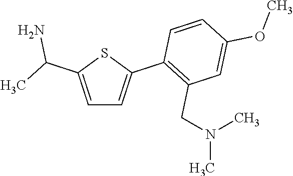 [SMILES: COc1ccc(-c2ccc(C(C)N)s2)c(CN(C)C)c1]

N-(1-(5-(2-((dimethylamino)methyl)-4-methoxyphenyl)thiophen-2-yl)ethyl)-2-methylpropane-2-sulfinamide, 50.0 mg (0.13 mmol), was dissolved in 3.0 mL of tetrahydrofuran and 1.0 mL of hydrochloric acid/1,4-dioxane (4.0 M). The resulting mixture was stirred at room temperature for 2 hours. The solvent was removed in vacuo and saturated aqueous sodium carbonate was added to adjust the pH value to 8. The resulting mixture was extracted with dichloromethane and the combined organic layers were dried over anhydrous sodium sulfate. The residue was purified with silica gel column chromatography to give 40 mg (84%) of the product as a light yellow solid. MS (ESIpos): m/z=291 [M+H]⁺. LC-MS [Method 4, gradient starting with water (0.05% NH₄HCO₃)-Acetonitrile, 5% B]: Rₜ=1.62 min.

### Step e

### N-[1-(5-{2-[(dimethylamino)methyl]-4-methoxyphenyl}thiophen-2-yl)ethyl]-6,7-dimethoxy-2-methylquinazolin-4-amine

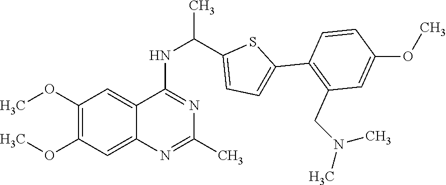

(1-(5-(2-((Dimethylamino)methyl)-4-methoxyphenyl)thiophen-2-yl)ethanamine, 40 mg (0.14 mmol), and 4-chloro-6,7-dimethoxy-2-methylquinazoline, 33 mg (0.14 mmol, commercially available), were dissolved into 3.0 mL of 2-propanol. The resulting mixture was stirred at 110° C. for 10 hours. After cooling to room temperature, the solvent was removed in vacuo and the residue was purified by preparative HPLC to give 0.9 mg of the product as a light yellow solid. MS (ESIpos): m/z=493 [M+H]⁺. LC-MS [Method 4, gradient starting with water (0.05% NH₄HCO₃)-Acetonitrile, 5% B]: Rₜ=2.08 min. ¹H-NMR (400 MHz, DMSO-d₆): δ [ppm]=1.71 (d, 3H), 2.11 (s, 6H), 2.43 (s, 3H), 3.34 (s, 2H), 3.76 (s, 3H), 3.87 (s, 6H), 5.90-6.00 (m, 1H), 6.85-6.87 (m, 1H), 7.00-7.05 (m, 4H), 7.28 (d, 1H), 7.65 (s, 1H), 8.14 (d, 1H).

### Example 440

### 3-(4-{[1-(5-{2-[(dimethylamino)methyl]phenyl}thiophen-2-yl)ethyl]amino}-2-methylquinazolin-6-yl)-1,1-dimethylurea

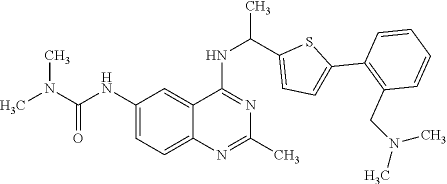 [SMILES: Cc1nc(NC(C)c2ccc(-c3ccccc3CN(C)C)s2)c2cc(NC(=O)N(C)C)ccc2n1]

N₄-(1-(5-(2-((dimethylamino)methyl)phenyl)thiophen-2-yl)ethyl)-2-methylquinazoline-4,6-diamine, 100 mg (0.24 mmol, described in example 423), and triethylamine, 121 mg (1.20 mmol), were dissolved into 3.0 mL of dichloromethane. Then dimethylcarbamic chloride, 129 mg (1.20 mmol), was added dropwise to the above solution at 0° C. and the resulting mixture was stirred at room temperature for 36 hours. The solvent was removed in vacuo and the residue was purified by preparative HPLC to give 18.9 mg (14%) of the product as a light yellow solid. MS (ESIpos): m/z=489 [M+H]⁺. LC-MS [Method 4, gradient starting with water (0.05% NH₄HCO₃)-Acetonitrile, 10% B]: Rₜ=1.93 min. ¹H-NMR (400 MHz, DMSO-d₆): δ [ppm]=1.64 (d, 3H), 2.04 (s, 6H), 2.38 (s, 3H), 2.88 (s, 6H), 3.29 (s, 2H), 5.85-5.89 (m, 1H), 7.00 (d, 1H), 7.10 (d, 1H), 7.19-7.26 (m, 2H), 7.29 (d, 1H), 7.35 (d, 1H), 7.44 (d, 1H), 7.63 (d, 1H), 8.13-8.17 (m, 3H), 8.30 (d, 1H), 8.43 (s, 1H).

### Example 441

### 1-benzyl-4-(4-{[1-(5-bromothiophen-2-yl)ethyl]amino}-2-methylquinazolin-6-yl)piperazin-2-one

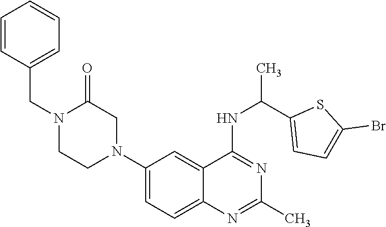 [SMILES: Cc1nc(NC(C)c2ccc(Br)s2)c2cc(N3CCN(Cc4ccccc4)C(=O)C3)ccc2n1]

### Step a

### methyl 5-fluoro-2-nitrobenzoate

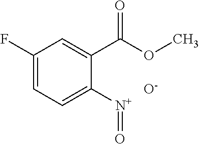

To a solution of 5-fluoro-2-nitrobenzoic acid, 4 g (21.6 mmol), in 40 mL of methanol was added 8.0 mL of sulfuric acid at 0° C. and the resulting mixture was stirred at 60° C. for 16 hours. After cooling to room temperature, aqueous potassium carbonate solution was added to adjust the pH value to 8 and the organic solvent was removed in vacuo. The resulting solution was extracted with ethyl acetate and the combined organic layers were dried over anhydrous sodium sulfate and concentrated in vacuo to give 3.6 g (82%) of the product as a red oil. MS (ESIpos): m/z=200 [M+H]⁺. LC-MS [Method 4, gradient starting with water (0.05% TFA)-Acetonitrile, 10% B]: Rₜ=1.01 min.

### Step b

### methyl 5-(4-benzyl-3-oxopiperazin-1-yl)-2-nitrobenzoate

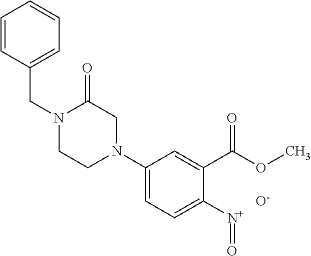

To a solution of methyl 5-fluoro-2-nitrobenzoate, 0.8 g (4.0 mmol), in 10 mL of N,N-dimethylformamide was added 1-benzylpiperazin-2-one, 1.53 g (8.0 mmol), and potassium carbonate, 1.11 g (8.0 mmol). The resulting mixture was stirred at reflux for 4 hours. After cooling to room temperature, water was added and the resulting solution was extracted with ethyl acetate. The combined organic layers were dried over anhydrous sodium sulfate and concentrated in vacuo to give 1.2 g (81%) of the product as a yellow solid. MS (ESIpos): m/z=370 [M+H]⁺. LC-MS [Method 4, gradient starting with water (0.05% TFA)-Acetonitrile, 10% B]: Rₜ=1.77 min.

### Step c

### methyl 2-amino-5-(4-benzyl-3-oxopiperazin-1-yl)benzoate

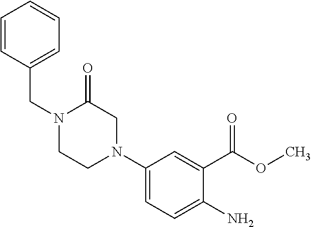 [SMILES: COC(=O)c1cc(N2CCN(Cc3ccccc3)C(=O)C2)ccc1N]

To a solution of methyl 5-(4-benzyl-3-oxopiperazin-1-yl)-2-nitrobenzoate, 1.20 g (3.2 mmol), in 20 mL of methanol was added palladium/carbon (10%), 0.34 g (0.3 mmol), and the resulting mixture was stirred at room temperature for 3 hours under hydrogen atmosphere (2 atm). The catalyst was removed by filtration and the filtrate was concentrated in vacuo to give 0.8 g (67%) of the product as a grey solid. MS (ESIpos): m/z=340 [M+H]⁺. LC-MS [Method 4, gradient starting with water (0.05% NH₄HCO₃)-Acetonitrile, 10% B]: Rₜ=1.64 min.

### Step d

### 6-(4-benzyl-3-oxopiperazin-1-yl)-2-methylquinazolin-4(3H)-one

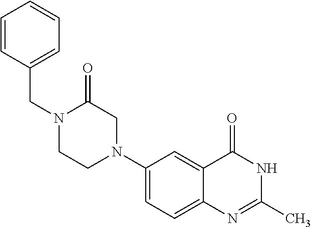 [SMILES: Cc1nc2ccc(N3CCN(Cc4ccccc4)C(=O)C3)cc2c(=O)[nH]1]

Dry hydrochloric acid gas was passed (until the clear solution observed) to a solution of methyl 2-amino-5-(4-benzyl-3-oxopiperazin-1-yl)benzoate, 0.8 g (2.4 mmol), in 20 mL of acetonitrile for 3 hours at room temperature. Then the resulting mixture was stirred at reflux for 10 hours and attained to room temperature. The precipitated solid was collected by filtration and the filter cake was dissolved with water. The resulting solution was neutralized with aqueous sodium bicarbonate solution (10%) and the precipitated solid was collected by filtration. The filter cake was washed with ice cold water and dried in air to give 0.68 g (79%) of the product as a white solid. MS (ESIpos): m/z=349 [M+H]⁺. LC-MS [Method 4, gradient starting with water (0.05% NH₄HCO₃)-Acetonitrile, 10% B]: Rₜ=2.02 min. ¹H-NMR (400 MHz, DMSO-d₆): δ [ppm]=2.30 (s, 3H), 3.36-3.38 (m, 2H), 3.56-3.59 (m, 2H), 3.97 (s, 2H), 4.61 (s, 2H), 7.26 7.29 (m, 3H), 7.32-7.37 (m, 3H), 7.46-7.53 (m, 2H), 12.04 (br, 1H).

### Step e

### 1-benzyl-4-(4-chloro-2-methylquinazolin-6-yl)piperazin-2-one

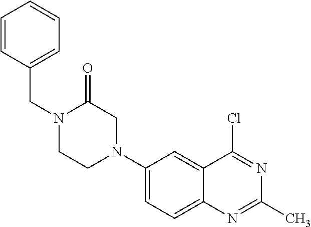 [SMILES: Cc1nc(Cl)c2cc(N3CCN(Cc4ccccc4)C(=O)C3)ccc2n1]

To a solution of 6-(4-benzyl-3-oxopiperazin-1-yl)-2-methylquinazolin-4(3H)-one, 300 mg (0.9 mmol), in 12 mL of toluene was added N,N-diisopropylethylamine, 0.6 mL (3.4 mmol), and the resulting mixture was stirred at 110° C. for 1 hour. Then 1.8 mL of phosphorus oxychloride was added to the above solution at 80° C. and the resulting mixture was stirred at this temperature for 3 hours. After cooling to room temperature, the solvent was removed in vacuo and the residue was dissolved in 10 mL of dichloromethane. Saturated potassium carbonate solution was added to adjust the pH value to 7-8 and the resulting mixture was extracted with dichloromethane. The combined organic layers were dried over anhydrous sodium sulfate and concentrated in vacuo to give 240 mg (55%) of the product as a brown solid. MS (ESIpos): m/z=367 [M+H]⁺. LC-MS [Method 4, gradient starting with water (0.1% HCOOH)-Acetonitrile, 10% B]: Rₜ=1.03 min.

### Step f

### 1-benzyl-4-(4-{[1-(5-bromothiophen-2-yl)ethyl]amino}-2-methylquinazolin-6-yl)piperazin-2-one

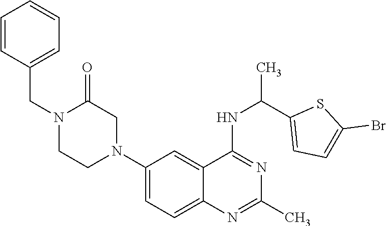 [SMILES: Cc1nc(NC(C)c2ccc(Br)s2)c2cc(N3CCN(Cc4ccccc4)C(=O)C3)ccc2n1]

To a solution of 1-benzyl-4-(4-chloro-2-methylquinazolin-6-yl)piperazin-2-one, 200 mg (0.7 mmol), in 4.0 mL of 2-propanol was added 1-(5-bromothiophen-2-yl)ethanamine, 134 mg (0.7 mmol). The resulting mixture was stirred at 110° C. for 16 hours under nitrogen atmosphere. After cooling to room temperature, the solvent was removed in vacuo and the residue was purified by silica gel column chromatography (dichloromethane:methanol=13:1) to give 120 mg (41%) of the product as a yellow solid. MS (ESIpos): m/z=537 [M+H]⁺. LC-MS [Method 4, gradient starting with water (0.05% NH₄HCO₃)-Acetonitrile, 10% B]: Rₜ=2.16 min. ¹H-NMR (400 MHz, DMSO-d₆): δ [ppm]=1.68 (d, 3H), 2.44 (s, 3H), 3.39-3.42 (m, 2H), 3.57-3.60 (m, 2H), 4.01 (s, 2H), 4.63 (s, 2H), 5.78-5.85 (m, 1H), 6.92-6.93 (m, 1H), 7.07-7.08 (m, 1H), 7.28-7.30 (m, 3H), 7.34-7.37 (m, 2H), 7.46-7.47 (m, 1H), 7.51-7.58 (m, 2H), 8.30 (br, 1H).

### Example 442

### N-[1-(5-{2-[(dimethylamino)methyl]-4-methylphenyl}thiophen-2-yl)ethyl]-6,7-dimethoxy-2-methylquinazolin-4-amine

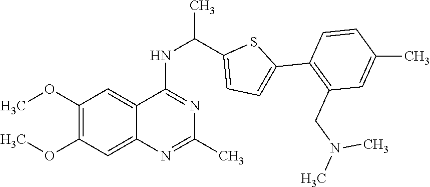

N-(1-(5-(4-bromo-2-((dimethylamino)methyl)phenyl)thiophen-2-yl)ethyl)-6,7-dimethoxy-2-methylquinazolin-4-amine, 20.0 mg (0.04 mmol, described in example 384), methylboronic acid, 6.6 mg (0.1 mmol), sodium carbonate, 15.7 mg (0.1 mmol) and 1,1′ bis(diphenylphosphino)ferrocenepalladium(II) chloride, 2.2 mg (0.004 mmol), were added into 1.2 mL of 1,4-dioxane/H₂O (v:v=5:1). The resulting mixture was stirred at 100° C. for 12 hours under nitrogen atmosphere. After cooling to room temperature, water was added and the resulting mixture was extracted with ethyl acetate. The combined organic layers were dried over anhydrous sodium sulfate and the solvent was removed in vacuo. The residue was purified by preparative HPLC to give 12.3 mg (69%) of the product as an off-white solid. MS (ESIpos): m/z=477 [M+H]⁺; LC-MS [Method 4, gradient starting with water (0.05% NH₄HCO₃)-Acetonitrile, 10% B]: Rₜ=3.25 min. ¹H-NMR (400 MHz, DMSO-d₆): δ [ppm]=1.71 (d, 3H), 2.10 (s, 6H), 2.31 (s, 3H), 2.43 (s, 3H), 3.33 (s, 2H), 3.87 (s, 6H), 5.94-5.97 (m, 1H), 7.05-7.13 (m, 4H), 7.24-7.26 (m, 2H), 7.65 (s, 1H), 8.14 (d, 1H).

### Example 443

### N-[1-(5-{4-cyclopropyl-2-[(dimethylamino)methyl]phenyl}thiophen-2-yl)ethyl]-6,7-dimethoxy-2-methylquinazolin-4-amine

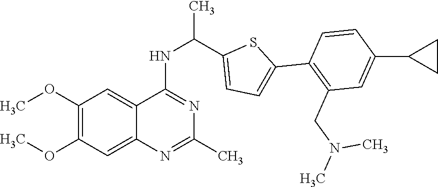 [SMILES: COc1cc2nc(C)nc(NC(C)c3ccc(-c4ccc(C5CC5)cc4CN(C)C)s3)c2cc1OC]

N-(1-(5-(4-bromo-2-((dimethylamino)methyl)phenyl)thiophen-2-yl)ethyl)-6,7-dimethoxy-2-methylquinazolin-4-amine, 20.0 mg (0.04 mmol, described in example 384), cyclopropylboronic acid, 9.5 mg (0.1 mmol), sodium carbonate, 15.7 mg (0.1 mmol) and 1,1′-bis(diphenylphosphino)ferrocenepalladium(II) chloride, 2.2 mg (0.004 mmol), were added into 1.2 mL of 1,4-dioxane/H₂O (v:v=5:1). The resulting mixture was stirred at 100° C. for 12 hours under nitrogen atmosphere. After cooling to room temperature, water was added and the resulting mixture was extracted with ethyl acetate. The combined organic layers were dried over anhydrous sodium sulfate and the solvent was removed in vacuo. The residue was purified by preparative HPLC to give 12.5 mg (67%) of the product as an off-white solid. MS (ESIpos): m/z=503 [M+H]⁺; LC-MS [Method 4, gradient starting with water (0.05% NH₄HCO₃)-Acetonitrile, 10% B]: Rₜ=3.45 min. ¹H-NMR (400 MHz, DMSO-d₆): δ [ppm]=0.65-0.66 (m, 2H), 0.95-0.96 (m, 2H), 1.71 (d, 3H), 1.89-1.96 (m, 1H), 2.10 (s, 6H), 2.43 (s, 3H), 3.32 (s, 2H), 3.87 (s, 6H), 5.93-5.97 (m, 1H), 6.95 (d, 1H), 7.04-7.05 (m, 2H), 7.11 (s, 1H), 7.12 (s, 1H), 7.23 (d, 1H), 7.65 (s, 1H), 8.13 (d, 1H).

### Example 444

### N-[1-(2-methyl-1,3-thiazol-4-yl)ethyl]quinazolin-4-amine

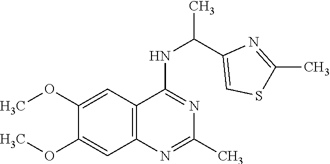

### Step a

### 1-(2-methyl-1,3-thiazol-4-yl)ethanamine

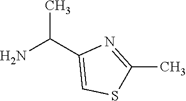

The title compound was prepared in analogy to 1-(5-bromothiophen-2-yl)ethanamine (INT-28) from 2-methyl-1,3-thiazole-4-carbaldehyde (commercially available, 1.00 g, 7.86 mmol) to give 300 mg of the title compound.

### Step b

### N-[1-(2-methyl-1,3-thiazol-4-yl)ethyl]quinazolin-4-amine

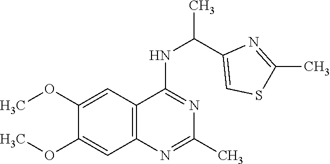

To a microwave vial were added 4-chloro-6,7-dimethoxy-2-methylquinazoline (50.0 mg, 209 μmol, commercially available), 1-(2-methyl-1,3-thiazol-4-yl)ethanamine (32.8 mg, 230 μmol), N,N-diisopropylethylamine (93 μL, 540 μmol) and DMSO (1.5 mL). The reaction mixture was heated to 130° C. during 2 hours in the microwave. Purification by preparative HPLC (acidic conditions) gave the title compound as a white solid (24.1 mg, 33%). ¹H-NMR (400 MHz, DMSO-d₆): δ [ppm]=8.08 (br d, 1H), 7.71 (s, 1H), 7.25 (d, 1H), 7.03 (s, 1H), 5.77 (quin, 1H), 3.88 (s, 3H), 3.87 (s, 3H), 2.64 (s, 3H), 2.39 (s, 3H), 1.60 (d, 3H). LC-MS (Method 7): m/z: [M+H]⁺=345, Rₜ=0.68 min.

### Example 445

### 6,7-dimethoxy-2-methyl-N-[1-(4-methyl-1,3-thiazol-2-yl)ethyl]quinazolin-4-amine

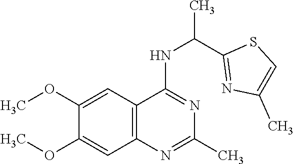 [SMILES: COc1cc2nc(C)nc(NC(C)c3nc(C)cs3)c2cc1OC]

### Step a

### 1-(4-methyl-1,3-thiazol-2-yl)ethanamine

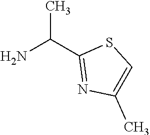

The title compound was prepared in analogy to 1-(5-bromothiophen-2-yl)ethanamine (INT-28) from 4-methyl-1,3-thiazole-2-carbaldehyde (commercially available, 1.00 g, 7.86 mmol) to give 530 mg of the title compound.

### Step b

### 6,7-dimethoxy-2-methyl-N-[1-(4-methyl-1,3-thiazol-2-yl)ethyl]quinazolin-4-amine

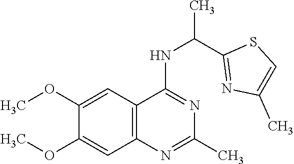 [SMILES: COc1cc2nc(C)nc(NC(C)c3nc(C)cs3)c2cc1OC]

To a microwave vial were added 4-chloro-6,7-dimethoxy-2-methylquinazoline (50.0 mg, 209 μmol, commercially available), 1-(4-methyl-1,3-thiazol-2-yl)ethanamine (32.8 mg, 230 μmol), N,N-diisopropylethylamine (93 μL, 540 μmol) and DMSO (1.5 mL). The reaction mixture was heated to 130° C. during 2 hours in the microwave. Purification by preparative HPLC (acidic conditions) gave the title compound as a light yellow solid (19.8 mg, 27%). ¹H-NMR (400 MHz, DMSO-d₆): δ [ppm]=8.32 (br d, 1H), 7.69 (s, 1H), 7.10 (d, 1H), 7.07 (s, 1H), 5.90 (quin, 1H), 3.88 (d, 6H), 2.40 (s, 3H), 2.35 (d, 3H), 1.71 (d, 3H). LC-MS (Method 7): m/z: [M+H]⁺=345, Rₜ=0.67 min.

### Example 446

### 3-(5-{1-[(6,7-dimethoxy-2-methylquinazolin-4-yl)amino]ethyl}thiophen-2-yl)-1-methyl-1H-pyrazole-5-carboxylic acid

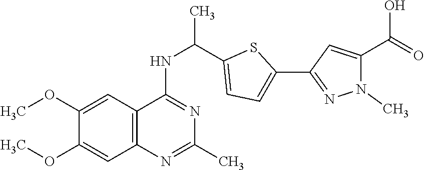 [SMILES: COc1cc2nc(C)nc(NC(C)c3ccc(-c4cc(C(=O)O)n(C)n4)s3)c2cc1OC]

Under argon, N-[1-(5-bromothiophen-2-yl)ethyl]-6,7-dimethoxy-2-methylquinazolin-4-amine (20.0 mg, 49.0 μmol, described in example 209), methyl 1-methyl-3-(4,4,5,5-tetramethyl-1,3,2-dioxaborolan-2-yl)-1H-pyrazole-5-carboxylate (13.0 mg, 49.0 μmol), K₂SO₃ (27.1 mg, 196 μmol) and Pd(PPh₃)₄ (2.83 mg, 2.45 μmol) in 1,4-dioxane (500 μL) and H₂O (100 μL) were stirred at 110° C. overnight. Brine was added, the mixture extracted with DCM and the solvent removed in vacuo. Purification by preparative HPLC (basic conditions) gave the title compound as a white solid (1.40 mg, 6%). ¹H-NMR (400 MHz, DMSO-d₆): δ [ppm]=8.20 (br d, 1H), 7.66 (s, 1H), 7.31 (d, 1H), 7.08 (s, 1H), 7.07 (s, 1H), 7.02 (dd, 1H), 5.92 (quin, 1H), 4.03 (s, 3H), 3.88 (s, 3H), 3.88 (s, 3H), 2.44 (s, 3H), 1.70 (d, 3H). LC-MS (Method 7): m/z: [M+H]⁺=454, Rₜ=0.73 min.

### Example 447

### tert-butyl [(5′-{1-[(6,7-dimethoxy-2-methylquinazolin-4-yl)amino]ethyl}-2,2′-bithiophen-5-yl)methyl]carbamate

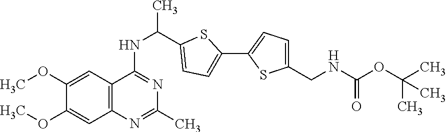 [SMILES: COc1cc2nc(C)nc(NC(C)c3ccc(-c4ccc(CNC(=O)OC(C)(C)C)s4)s3)c2cc1OC]

Under argon, N-[1-(5-bromothiophen-2-yl)ethyl]-6,7-dimethoxy-2-methylquinazolin-4-amine (20.0 mg, 49.0 μmol, described in example 209), (5-{[(tert-butoxycarbonyl)amino]methyl}thiophen-2-yl)boronic acid (12.6 mg, 49.0 μmol), K₂CO₃ (27.1 mg, 196 μmol) and Pd(PPh₃)₄ (2.83 mg, 2.45 μmol) in 1,4-dioxane (500 μL) and H₂O (100 μL) were stirred at 110° C. overnight. H₂O was added, the mixture extracted with DCM and the solvent removed in vacuo. Purification by preparative HPLC (basic conditions) gave the title compound as a light yellow solid (11.1 mg, 41%). ¹H-NMR (400 MHz, DMSO-d₆): δ [ppm]=8.12 (d, 1H), 7.64 (s, 1H), 7.50 (t, 1H), 7.07 (d, 1H), 7.06 (s, 1H), 7.03-7.00 (m, 2H), 6.82 (d, 1H), 5.89 (quin, 1H), 4.22 (br d, 2H), 3.88 (s, 6H), 2.43 (s, 3H), 1.69 (d, 3H), 1.39 (s, 9H). LC-MS (Method 9): m/z: [M+H]⁺=541, Rₜ=1.09 min.

### Example 448

### 7-methoxy-2-methyl-6-[2-(methylsulfonyl)ethoxy]-N-{(1R)-1-[3-(1H-pyrazol-4-yl)phenyl]ethyl}quinazolin-4-amine

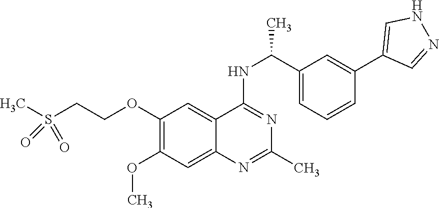 [SMILES: COc1cc2nc(C)nc(N[C@H](C)c3cccc(-c4cn[nH]c4)c3)c2cc1OCCS(C)(=O)=O]

This compound was synthesized from 7-methoxy-2-methyl-4-({(1R)-1-[3-(1H-pyrazol-4-yl)phenyl]ethyl}amino)quinazolin-6-ol (described in example 345) by the same method as described in example 355 to give 9.9 mg of the product as a brown solid. MS (ESIpos): m/z=482 [M+H]⁺. LC-MS [Method 4, gradient starting with water (0.05% TFA)-Acetonitrile, 5% B]: Rₜ=1.04 min. ¹H-NMR (400 MHz, DMSO-d₆): δ [ppm]=1.23 (s, 3H), 1.56-1.58 (d, 3H), 2.34 (s, 3H), 2.87 (s, 3H), 3.73 (m, 2H), 3.90 (s, 3H), 4.55-4.58 (m, 2H), 5.58-5.61 (m, 1H), 7.01 (s, 1H), 7.28-7.31 (d, 2H), 7.39-7.40 (d, 1H), 7.65-7.66 (d, 2H), 7.82-7.84 (d, 1H), 7.92 (s, 1H), 8.23 (s, 1H), 9.20 (br, 1H).

### Example 449

### tert-butyl [5-chloro-2-(5-{1-[(6,7-dimethoxy-2-methylquinazolin-4-yl)amino]ethyl}thiophen-2-yl)benzyl]carbamate

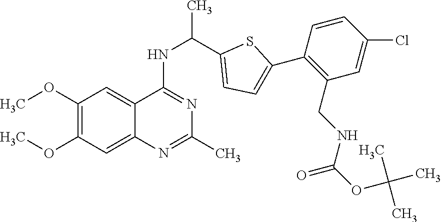 [SMILES: COc1cc2nc(C)nc(NC(C)c3ccc(-c4ccc(Cl)cc4CNC(=O)OC(C)(C)C)s3)c2cc1OC]

### Step a

### 1-(2-bromo-5-chlorophenyl)methanamine

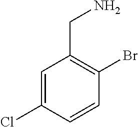

This compound was synthesized by the same method as described in example 408 (step a) to give 1.17 g (38%) of the product as the light yellow oil. MS (ESIpos): m/z=220 [M+H]⁺. LC-MS [Method 4, Water (0.1% HCOOH)-Acetonitrile, 10% B]: Rₜ=0.34 min.

### Step b

### tert-butyl (2-bromo-5-chlorobenzyl)carbamate

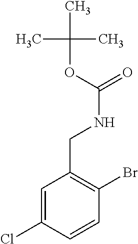 [SMILES: CC(C)(C)OC(=O)NCc1cc(Cl)ccc1Br]

This compound was synthesized by the same method as described in example 429 (step a) to give 360 mg (21%) of the product as a light yellow solid. MS (ESIpos): m/z=320 [M+H]⁺. LC-MS [Method 4, Water (0.05% NH₄HCO₃)-Acetonitrile, 10% B]: Rₜ=2.16 min.

### Step c

### tert-butyl[5-chloro-2-(4,4,5,5-tetramethyl-1,3,2-dioxaborolan-2-yl)benzyl]carbamate

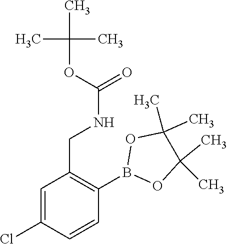 [SMILES: CC(C)(C)OC(=O)NCc1cc(Cl)ccc1B1OC(C)(C)C(C)(C)O1]

This compound was synthesized by the same method as described in example 429 (step b) to give 190 mg (46%) of the product as a light yellow oil. MS (ESIpos): m/z=368 [M+H]⁺. LC-MS [Method 4, Water (0.05% TFA)-Acetonitrile, 5% B]: Rₜ=1.34 min.

### Step d

### tert-butyl [5-chloro-2-(5-{1-[(6,7-dimethoxy-2-methylquinazolin-4-yl)amino]-ethyl}thiophen-2-yl)benzyl]carbamate

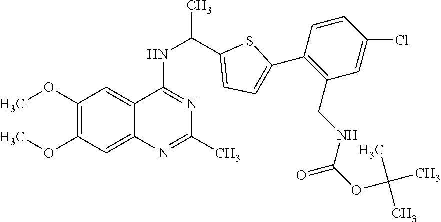 [SMILES: COc1cc2nc(C)nc(NC(C)c3ccc(-c4ccc(Cl)cc4CNC(=O)OC(C)(C)C)s3)c2cc1OC]

This compound was synthesized by the same method as described in example 429 (step c) to give 160 mg (82%) of the product as an off-white solid. MS (ESIpos): m/z=569 [M+H]⁺. LC-MS [Method 4, Water (0.05% TFA)-Acetonitrile, 5% B]: Rₜ=1.58 min. ¹H-NMR (400 MHz, DMSO-d₆): δ [ppm]=1.38 (s, 9H), 1.72 (d, 3H), 2.43 (s, 3H), 3.87 (s, 6H), 4.22 (d, 2H), 5.96-5.97 (m, 1H), 7.05-7.07 (m, 2H), 7.10-7.11 (m, 1H), 7.33-7.34 (m, 3H), 7.49-7.51 (m, 1H), 7.64 (s, 1H), 8.16 (d, 1H).

### Example 450

### tert-butyl [2-chloro-6-(5-{1-[(6,7-dimethoxy-2-methylquinazolin-4-yl)amino]ethyl}-thiophen-2-yl)benzyl]carbamate

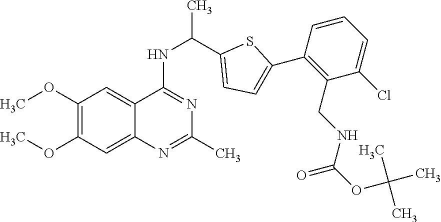 [SMILES: COc1cc2nc(C)nc(NC(C)c3ccc(-c4cccc(Cl)c4CNC(=O)OC(C)(C)C)s3)c2cc1OC]

### Step a

### 1-(2-bromo-6-chlorophenyl)methanamine

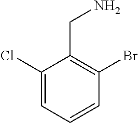

This compound was synthesized by the same method as described in example 408 (step a) to give 1.00 g (32%) of the product as the light yellow oil. MS (ESIpos): m/z=220 [M+H]⁺. LC-MS [Method 4, Water (0.1% HCOOH)-Acetonitrile, 10% B]: Rₜ=0.54 min.

### Step b

### tert-butyl (2-bromo-6-chlorobenzyl)carbamate

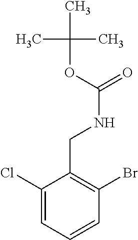 [SMILES: CC(C)(C)OC(=O)NCc1c(Cl)cccc1Br]

This compound was synthesized by the same method as described in example 429 (step a) to give 1.17 g (81%) of the product as a light yellow solid. MS (ESIpos): m/z=320 [M+H]⁺. LC-MS [Method 4, Water (0.05% TFA)-Acetonitrile, 5% B]: Rₜ=1.18 min.

### Step c

### tert-butyl[6-chloro-2-(4,4,5,5-tetramethyl-1,3,2-dioxaborolan-2-yl)benzyl]carbamate

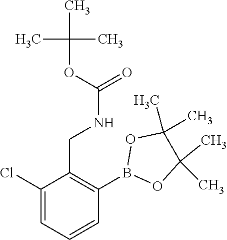 [SMILES: CC(C)(C)OC(=O)NCc1c(Cl)cccc1B1OC(C)(C)C(C)(C)O1]

This compound was synthesized by the same method as described in example 429 (step b) to give 400 mg (33%) of the product as a light yellow oil. MS (ESIpos): m/z=368 [M+H]⁺. LC-MS [Method 4, Water (0.1% HCOOH)-Acetonitrile, 10% B]: Rₜ=1.43 min.

### Step d

### tert-butyl [2-chloro-6-(5-{1-[(6,7-dimethoxy-2-methylquinazolin-4-yl)amino]ethyl}-thiophen-2-yl)benzyl]carbamate

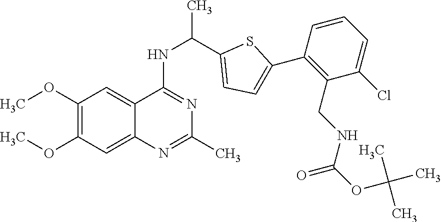 [SMILES: COc1cc2nc(C)nc(NC(C)c3ccc(-c4cccc(Cl)c4CNC(=O)OC(C)(C)C)s3)c2cc1OC]

This compound was synthesized by the same method as described in example 429 (step c) to give 250 mg (79%) of the product as an off-white solid. MS (ESIpos): m/z=569 [M+H]⁺. LC-MS [Method 4, Water (0.05% TFA)-Acetonitrile, 5% B]: Rₜ=1.55 min. ¹H-NMR (400 MHz, DMSO-d₆): δ [ppm]=1.36 (s, 9H), 1.71 (d, 3H), 2.43 (s, 3H), 3.87 (s, 6H), 4.19 (d, 2H), 5.91-6.02 (m, 1H), 7.05-7.07 (m, 2H), 7.09-7.10 (m, 2H), 7.30-7.33 (m, 2H), 7.45-7.48 (m, 1H), 7.64 (s, 1H), 8.16 (d, 1H).

### Example 451

### 7-bromo-N-[(1R)-1-(3-chlorophenyl)ethyl]-2-methylquinazolin-4-amine

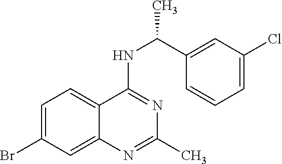 [SMILES: Cc1nc(N[C@H](C)c2cccc(Cl)c2)c2ccc(Br)cc2n1]

A solution of 7-bromo-2-methylquinazolin-4-ol (520 mg, 2.17 mmol, prepared as described in patent US2004/29901 A1, 2004, page 13), 2,4,6-triisopropylbenzenesulfonyl chloride (1.32 g, 4.35 mmol), triethylamine (1.21 mL, 8.70 mmol) and 4-dimethylaminopyridine (53.2 mg, 0.44 mmol) in DMF (18.6 mL) was stirred at ambient temperature for 3 hours. (1R)-1-(3-chlorophenyl)ethanamine (812 mg, 5.22 mmol, commercially available) was added and the reaction was stirred overnight at room temperature. The solvent was distilled off under reduced pressure and the residue was extracted with ethylacetate (3×)/water. The combined organic layers were washed with brine, dried over sodium sulfate, filtered and the solvent evaporated. The residue was purified via Isolera flash chromatography (100 g silica, eluent hexanes/ethylacetate) to yield the title compound (713 mg, 78%). ¹H-NMR (400 MHz, DMSO-d₆): δ [ppm]=8.51 (d, 1H), 8.34 (d, 1H), 7.78 (d, 1H), 7.64 (dd, 1H), 7.50 (t, 1H), 7.44-7.38 (m, 1H), 7.35 (t, 1H), 7.31-7.25 (m, 1H), 5.59 (quin, 1H), 2.39 (s, 3H), 1.56 (d, 3H). LC-MS (Method 8): m/z: [M+H]⁺=378, Rₜ=0.93 min.

### Example 452

### N-[1-(5-bromothiophen-2-yl)ethyl]-2-methyl-6-nitroquinazolin-4-amine

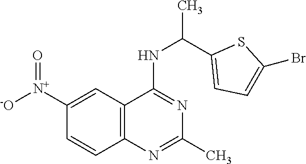

### Step a

### 2-methyl-6-nitroquinazolin-4(3H)-one

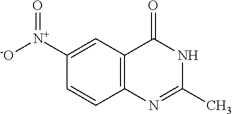

Dry HCl gas was passed (until the clear solution observed) to a solution of methyl 2-amino-5-nitrobenzoate, 5.00 g (25.5 mmol), in 100 mL of acetonitrile at room temperature for 3 hours and the resulting mixture was heated to reflux for overnight. After cooling to room temperature, the precipitated solid was collected by filtration. The filter cake was washed with acetonitrile and re-dissolved with water. The resulting solution was neutralized with saturated sodium bicarbonate solution and the precipitated solid was collected by filtration. The filter cake was washed with ice cold water and dried in air to give 4.75 g (90%) of the product as a light brown solid. MS (ESIpos): m/z=206 [M+H]⁺; LC-MS [Method 4, gradient starting with water (0.05% TFA)-Acetonitrile, 5% B]: Rₜ=0.70 min.

### Step b

### 4-chloro-2-methyl-6-nitroquinazoline

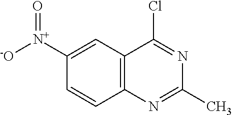

2-Methyl-6-nitroquinazolin-4(3H)-one, 2.00 g (9.7 mmol), and 4.82 mL of N,N-diisopropylethylamine were dissolved into 50 mL of toluene. The resulting mixture was stirred at 120° C. for 1 hour. After cooling to 80° C., 1.00 mL of phosphorus oxychloride (10.7 mmol), was added dropwise to the above solution and the mixture was heated at this temperature for 3 hours. After cooling to room temperature, the solvent was removed in vacuo and the residue was re-dissolved with dichloromethane. Aq. potassium carbonate solution was added to adjust pH value to 7-8 and the resulting mixture was extracted with ethyl acetate. The combined organic layers were dried over anhydrous sodium sulfate and the solvent was removed in vacuo. The residue was purified with silica gel column chromatography to give 1.50 g (68%) of the product as a yellow solid. MS (ESIpos): m/z=224 [M+H]⁺; LC-MS [Method 4, gradient starting with water (0.1% HCOOH)-Acetonitrile, 5% B]: Rₜ=1.01 min.

### Step c

### N-[1-(5-bromothiophen-2-yl)ethyl]-2-methyl-6-nitroquinazolin-4-amine

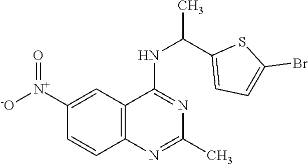

4-Chloro-2-methyl-6-nitroquinazoline, 1.50 g (6.7 mmol), and 1-(5-bromothiophen-2-yl)ethanamine, 1.38 g (6.7 mmol), were dissolved into 30 mL of 2-propanol. The resulting mixture was stirred at 110° C. for 10 hours. After cooling to room temperature, the solvent was removed in vacuo to give 2.50 g (crude) of the product as a yellow solid and it was used directly for next step without further purification. MS (ESIpos): m/z=393 [M+H]⁺; LC-MS [Method 4, gradient starting with water (0.05% TFA)-Acetonitrile, 5% B]: Rₜ=0.95 min. ¹H-NMR (400 MHz, DMSO-d₆): δ [ppm]=1.69 (d, 3H), 2.53 (s, 3H), 5.78-5.85 (m, 1H), 6.97 (d, 1H), 7.09 (d, 1H), 7.76 (d, 1H), 8.45 (d, 1H), 9.19 (d, 1H), 9.42 (s, 1H).

### Example 453

### methyl 4-{[(1R)-1-(3-chlorophenyl)ethyl]amino}-2-methylquinazoline-7-carboxylate

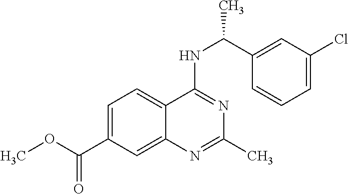 [SMILES: COC(=O)c1ccc2c(N[C@H](C)c3cccc(Cl)c3)nc(C)nc2c1]

To a solution of 7-bromo-N-[(1R)-1-(3-chlorophenyl)ethyl]-2-methylquinazolin-4-amine (described in example 451, 700 mg, 1.86 mmol) in methanol/THF (10:1, 33 mL) was added 1,1-bis-(diphenylphosphino)-ferrocen-palladium(II)dichloride (151 mg, 0.19 mmol) and trimethylamine (0.52 mL) and reacted in an autoclave with carbon monoxide (15.5 bar) at 80° C. for 25 hours. The reaction mixture was evaporated and the crude product was purified by Isolera flash chromatography (110 g NH phase; eluent gradient hexanes:ethyl acetate 5-90%). The title compound was obtained in 75% yield (523 mg). 1H-NMR (400 MHz, DMSO-d₆): δ [ppm]=8.61 (d, 1H), 8.50 (d, 1H), 8.12 (d, 1H), 7.94 (dd, 1H), 7.52 (t, 1H), 7.45-7.40 (m, 1H), 7.35 (t, 1H), 7.31-7.26 (m, 1H), 5.60 (quin, 1H), 3.91 (s, 3H), 2.42 (s, 3H), 1.58 (d, 3H). LC-MS (Method 7): m/z: [M+H]⁺=356.1, Rₜ=0.82 min.

### Example 454

### 3-amino-3-[2-(5-{1-[(6,7-dimethoxy-2-methylquinazolin-4-yl)amino]ethyl}-3-thienyl)phenyl]propanoic acid

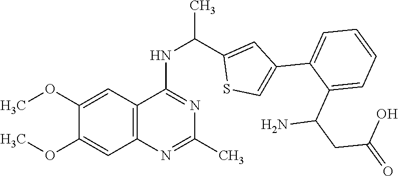 [SMILES: COc1cc2nc(C)nc(NC(C)c3cc(-c4ccccc4C(N)CC(=O)O)cs3)c2cc1OC]

### Step a

### 3-(2-bromophenyl)-3-[(tert-butoxycarbonyl)amino]propanoic acid

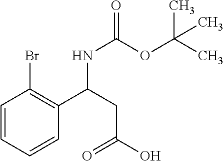 [SMILES: CC(C)(C)OC(=O)NC(CC(=O)O)c1ccccc1Br]

A solution of 3-amino-3-(2-bromophenyl)propanoic acid (59.8 mg, 245 μmol), di-tert-butyl dicarbonate (53.5 mg, 245 μmol) and N,N-diisopropylethylamine (47 μl, 270 μmol) in THE (1.0 mL) and H₂O (200 μL) was stirred at room temperature for 2 hours. The solvent was removed in vacuo to give the title compound which was used directly in step c. LC-MS (Method 10): m/z: [M+H]⁺=344, Rₜ=0.67 min.

### Step b

### (5-{1-[(6,7-dimethoxy-2-methylquinazolin-4-yl)amino]ethyl}thiophen-3-yl)boronic acid

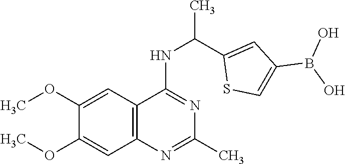 [SMILES: COc1cc2nc(C)nc(NC(C)c3cc(B(O)O)cs3)c2cc1OC]

Under argon, N-[1-(4-bromothiophen-2-yl)ethyl]-6,7-dimethoxy-2-methylquinazolin-4-amine (100 mg, 245 μmol, described in example 264), tetrahydroxydiborane (73.2 mg, 816 μmol), potassium acetate (80.1 mg, 816 μmol), XPhos (2.34 mg, 4.90 μmol) and XPhos Pd G2 (2.18 mg, 2.78 μmol) in degassed ethanol (5.0 mL) were stirred at 100° C. in the microwave for 1 hour. The solvent was removed in vacuo to give the title compound which was used directly in step c. LC-MS (Method 10): m/z: [M+H]⁺=373, Rₜ=0.81 min.

### Step c

### 3-[(tert-butoxycarbonyl)amino]-3-[2-(5-{1-[(6,7-dimethoxy-2-methylquinazolin-4-yl)amino]ethyl}thiophen-3-yl)phenyl]propanoic acid

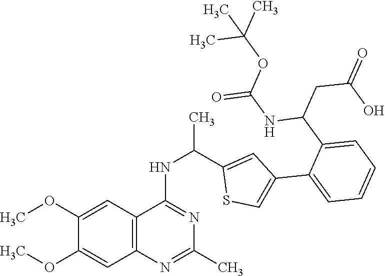 [SMILES: COc1cc2nc(C)nc(NC(C)c3cc(-c4ccccc4C(CC(=O)O)NC(=O)OC(C)(C)C)cs3)c2cc1OC]

Under argon, (5-{1-[(6,7-dimethoxy-2-methylquinazolin-4-yl)amino]ethyl}thiophen-3-yl)boronic acid (91.4 mg, 245 μmol), 3-(2-bromophenyl)-3-[(tert-butoxycarbonyl)amino]propanoic acid (84.3 mg, 245 μmol), K₂SO₃ (135 mg, 980 μmol) and Pd(PPh₃)₄ (28.3 mg, 24.5 μmol) in 1,4-dioxane (2.5 mL) and H₂O (500 μL) were stirred at 110° C. over the weekend. H₂O was added, the mixture extracted with DCM and the solvent removed in vacuo. Purification by preparative HPLC (basic conditions) gave the title compound (136 mg, 35%) which was used directly in step d.

### Step d

### 3-amino-3-[2-(5-{1-[(6,7-dimethoxy-2-methylquinazolin-4-yl)amino]ethyl}-3-thienyl)phenyl]propanoic acid

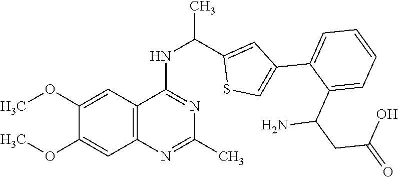 [SMILES: COc1cc2nc(C)nc(NC(C)c3cc(-c4ccccc4C(N)CC(=O)O)cs3)c2cc1OC]

To a solution of 3-[(tert-butoxycarbonyl)amino]-3-[2-(5-{1-[(6,7-dimethoxy-2-methylquinazolin-4-yl)amino]ethyl}thiophen-3-yl)phenyl]propanoic acid (136 mg, 229 μmol) in DCM (2.0 mL) was slowly added trifluoroacetic acid (180 μl, 2.3 mmol) and the reaction mixture stirred at room temperature overnight. Trifluoroacetic acid (180 μl, 2.3 mmol) was slowly added and the reaction mixture stirred for 3 hours. The solvent was removed in vacuo. Purification by preparative HPLC (basic conditions) gave the title compound as a white solid (19.6 mg, 17%). ¹H-NMR (400 MHz, DMSO-d₆): δ [ppm]=8.23 (dd, 1H), 7.69-7.64 (m, 2H), 7.41 (td, 1H), 7.36-7.30 (m, 2H), 7.27-7.21 (m, 1H), 7.14 (dt, 1H), 7.05 (s, 1H), 5.97 (quin, 1H), 4.40 (dt, 1H), 3.87 (s, 3H), 3.86 (s, 3H), 2.43 (d, 3H), 2.39 (ddd, 1H), 2.26 (ddd, 1H), 1.71 (d, 3H). LC-MS (Method 10): m/z: [M+H]⁺=493, Rₜ=0.73 min.

### Example 455

### N-[1-{5-[2-(2-aminopropan-2-yl)phenyl]thiophen-2-yl}ethyl]-6,7-dimethoxy-2-methylquinazolin-4-amine

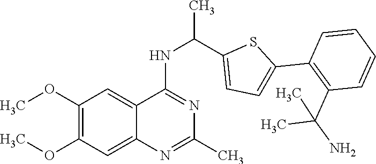 [SMILES: COc1cc2nc(C)nc(NC(C)c3ccc(-c4ccccc4C(C)(C)N)s3)c2cc1OC]

### Step a

### 2-[2-(5-{1-[(6,7-dimethoxy-2-methylquinazolin-4-yl)amino]ethyl}-2-thienyl)phenyl]-2-methylpropanoic acid

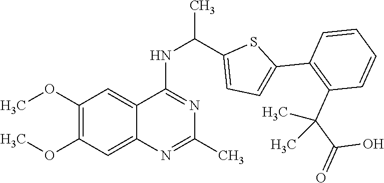 [SMILES: COc1cc2nc(C)nc(NC(C)c3ccc(-c4ccccc4C(C)(C)C(=O)O)s3)c2cc1OC]

To a solution of methyl 2-[2-(5-{1-[(6,7-dimethoxy-2-methylquinazolin-4-yl)amino]ethyl}-2-thienyl)phenyl]-2-methylpropanoate, 208 mg (0.41 mmol, described in example 436), in 10 mL of methanol was added 1.0 mL of sodium hydroxide (2.0 M), at 0° C. and the resulting mixture was stirred at 60° C. for 60 hours. After cooling to room temperature, the solvent was removed in vacuo and the residue was re-dissolved with water. The resulting mixture was washed with ethyl acetate and the pH value was adjusted to 4 with hydrochloric acid (6.0 M). The resulting solution was extracted with ethyl acetate and the combined organic layers were concentrated in vacuo to give 120 mg (59%) of the product as yellow solid. MS (ESIpos): m/z=492 [M+H]⁺. LC-MS [Method 4, Water (0.05% NH₄HCO₃)-Acetonitrile, 10% B]: Rₜ=1.35 min.

### Step b

### N-[1-{5-[2-(2-aminopropan-2-yl)phenyl]thiophen-2-yl}ethyl]-6,7-dimethoxy-2-methylquinazolin-4-amine

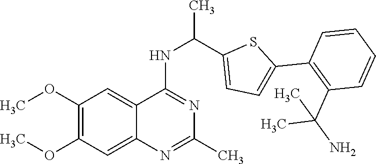 [SMILES: COc1cc2nc(C)nc(NC(C)c3ccc(-c4ccccc4C(C)(C)N)s3)c2cc1OC]

To a solution of 2-[2-(5-{1-[(6,7-dimethoxy-2-methylquinazolin-4-yl)amino]ethyl}-2-thienyl)phenyl]-2-methylpropanoic acid, 85 mg (0.17 mmol), in 5.0 mL of toluene were added diphenyl phosphoroazidate, 95 mg (0.35 mmol), and triethylamine, 35 mg (0.35 mmol). The resulting mixture was stirred at 50° C. for 16 hours under nitrogen atmosphere. After cooling to room temperature, the solvent was removed in vacuo and the residue was re-dissolved with ethyl acetate. The resulting mixture was washed with water, brine, dried over anhydrous sodium sulfate and concentrated in vacuo. The residue was dissolved in 5.0 mL of 1,4-dioxane and to this solution was added 1.0 mL of hydrochloric acid (3.0 M). The resulting mixture was stirred at room temperature for 5 hours. Saturated sodium carbonate solution was added to adjust the pH value to 8 and the solvent was removed in vacuo. The residue was purified by preparative HPLC [Mobile Phase A: Water (0.1% NH₄HCO₃), Mobile Phase B: Acetonitrile; Gradient: 15% B to 45% B in 7 min] to give 2.3 mg of the product as an off-white solid. MS (ESIpos): m/z=463 [M+H]⁺. LC-MS [Method 4, Water (0.05% NH₄HCO₃)-Acetonitrile, 10% B]: Rₜ=1.92 min. ¹H-NMR (400 MHz, DMSO-d₆): δ [ppm]=1.30 (s, 6H), 1.70 (d, 3H), 2.32 (s, 3H), 3.86 (s, 6H), 5.89-5.93 (m, 1H), 6.53 (d, 1H), 6.87 (d, 1H), 7.03 (s, 1H), 7.06 (d, 1H), 7.17 (t, 1H), 7.30 (t, 1H), 7.64 (s, 1H), 7.73 (d, 1H), 8.12 (d, 1H).

### Example 456

### {[2-(5-{1-[(6,7-dimethoxy-2-methylquinazolin-4-yl)amino]ethyl}thiophen-2-yl)benzyl](methyl)amino}acetonitrile

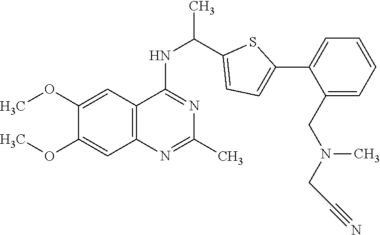 [SMILES: COc1cc2nc(C)nc(NC(C)c3ccc(-c4ccccc4CN(C)CC#N)s3)c2cc1OC]

A solution of N-[1-(5-{2-[(dimethylamino)methyl]phenyl}thiophen-2-yl)ethyl]-6,7-dimethoxy-2-methylquinazolin-4-amine (40.0 mg, 86.5 μmol), trimethylsilylcyanide (69 μL, 520 μmol) and rose bengal (880 μg, 0.86 μmol) in acetonitrile (940 μL) was lit with a fluorescent household lightbulb and stirred under an O₂ atmosphere overnight. The reaction mixture was quenched with NaHCO₃(aq., sat.) and extracted with DCM. Purification by preparative HPLC (basic conditions) gave the title compound (6.1 mg, 13%). ¹H-NMR (400 MHz, DMSO-d₆): δ [ppm]=8.16 (br d, 1H), 7.65 (s, 1H), 7.44-7.28 (m, 4H), 7.12-7.07 (m, 2H), 7.05 (s, 1H), 5.97 (quin, 1H), 3.87 (s, 3H), 3.87 (s, 3H), 3.61 (d, 3H), 2.43 (s, 3H), 2.20 (s, 2H), 2.11 (s, 1H), 1.72 (d, 3H). LC-MS (Method 10): m/z: [M+H]⁺=488, Rₜ=1.35 min.

### Example 457

### 1-(4-{[1-(5-{2-[(dimethylamino)methyl]phenyl}thiophen-2-yl)ethyl]amino}-2-methylquinazolin-6-yl)-3-methylurea

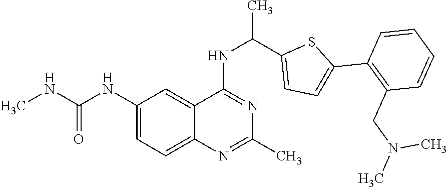 [SMILES: CNC(=O)Nc1ccc2nc(C)nc(NC(C)c3ccc(-c4ccccc4CN(C)C)s3)c2c1]

N₄-[1-(5-{2-[(dimethylamino)methyl]phenyl}-2-thienyl)ethyl]-2-methylquinazoline-4,6-diamine, 100 mg (0.24 mmol, described in example 423), was dissolved in 3.0 mL of dichloromethane. Triethylamine, 73 mg (0.72 mmol), and methylcarbamic chloride, 67 mg (0.14 mmol), were added. The resulting mixture was stirred at room temperature for 3 days. After evaporation in vacuo, the residue was purified by preparative HPLC (Column: Xbridge Prep C18, 5 μm, 19*150 mm; Mobile Phase A: Waters (0.1% HCOOH), Mobile Phase B: ACN; Flow rate: 20 mL/min; Gradient: 10% B to 36% B in 8 min; 254 nm & 220 nm), the solvent was lyophilized to give 34.3 mg (26%) of the product as a yellow solid. MS (ESIpos): m/z=475 [M+H]⁺. ¹H-NMR (400 MHz, DMSO-d₆): δ [ppm]=8.62 (s, 1H), 8.43-8.32 (m, 1H), 8.17 (s, 3H), 7.70 (dd, 1H), 7.52 (d, 1H), 7.47-7.25 (m, 5H), 7.18 (d, 1H), 7.08 (d, 1H), 6.19 (br d, 1H), 5.95 (br t, 1H), 2.68 (d, 3H), 2.45 (s, 3H), 2.13 (s, 6H), 1.72 (d, 3H).

### Example 458

### 1-benzyl-4-(4-{[1-(5-{2-[(dimethylamino)methyl]phenyl}thiophen-2-yl)ethyl]amino}-2-methylquinazolin-6-yl)piperazin-2-one

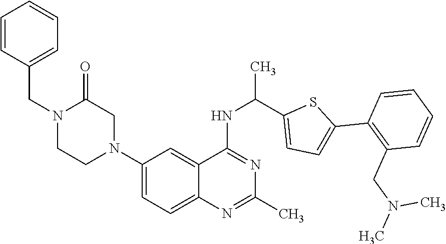 [SMILES: Cc1nc(NC(C)c2ccc(-c3ccccc3CN(C)C)s2)c2cc(N3CCN(Cc4ccccc4)C(=O)C3)ccc2n1]

1-benzyl-4-(4-{[1-(5-bromothiophen-2-yl)ethyl]amino}-2-methylquinazolin-6-yl)piperazin-2-one, 70 mg (130.5 μmol, described in example 441), 2-((dimethylamino)methyl)phenylboronic acid, 23.4 mg (130.5 μmol), tetrakis(triphenylphosphine)palladium(0), 15.1 mg (13 μmol), and potassium carbonate, 72.1 mg (521.9 μmol), were dissolved in 4.8 mL of 1,4-dioxane/water (v:v=5:1) at room temperature. The resulting mixture was stirred at 100° C. for 10 hours under nitrogen. The solvent was removed in vacuo the residue was purified by preparative HPLC (Column: XBridge Prep C18 OBD Column 19×150 mm 5 μm; Mobile Phase A: Water (0.1% HCOOH), Mobile Phase B: ACN; Flow rate: 20 mL/min; Gradient: 30% B to 70% B in 8 min; Detector: 254 nm, 220 nm) to give 7.2 mg (9%) of the product as a light yellow solid. MS (ESIpos): m/z=591 [M+H]⁺.

### EXPERIMENTAL SECTION—BIOLOGICAL ASSAYS

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

6,7-dimethoxy-N-[(1R)-1-(1-naphthyl)ethyl]quinazolin-4-amine, which was used to calibrate the assay, was prepared as follows:

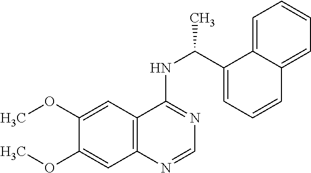 [SMILES: COc1cc2ncnc(N[C@H](C)c3cccc4ccccc34)c2cc1OC]

To 4-chloro-6,7-dimethoxyquinazoline (100 mg, 0.445 mmol, commercially available) in 1.7 mL DMSO was added (1R)-1-(1-naphthyl)ethanamine (76 mg, 0.445 mmol, commercially available) and N-ethyl-N-isopropylpropan-2-amine (202 μl, 1.16 mmol). The reaction was stirred at 100° C. overnight, cooled to ambient temperature and filtered. After removal of the solvent under reduced pressure the crude product was purified via HPLC chromatography to yield the title compound (118 mg, 73%). ¹H-NMR (400 MHz, DMSO-d₆), δ [ppm]=1.72 (3H), 3.90 (6H), 6.32-6.41 (1H), 7.09 (1H), 7.46-7.58 (3H), 7.64-7.69 (1H), 7.78 (2H), 7.92-7.97 (1H), 8.18-8.24 (2H), 8.28 (1H).

The in vitro activity of the compounds of the present invention can be demonstrated in the following assays:

Biochemical assay 1: K-Ras^{G12C} Interaction Assay with hSOS1

This assay quantifies the equilibrium interaction of human SOS1 with K-Ras^{Gl2C}. Detection of the interaction is achieved by measuring homogenous time-resolved fluorescence resonance energy transfer (HTRF) from antiGST-Europium (FRET donor) bound to GST-K-Ras^{G12C} to anti-6His-XL665 bound to His-tagged hSOS1 (FRET-acceptor).

The assay buffer contained 5 mM HEPES pH 7.4 (Applichem), 150 mM NaCl (Sigma), 10 mM EDTA (Promega), 1 mM DTT (Thermofisher), 0.05% BSA Fraction V, pH 7.0, (ICN Biomedicals), 0.0025% (v/v) Igepal (Sigma) and 100 mM KF (FLUKA).

The expression and purification of N-terminal GST-tagged human K-RasG120 and N-terminal His-tagged human SOS1 is described below. Concentrations of protein batches used were optimized to be within the linear range of the HTRF signal. A Ras working solution was prepared in assay buffer containing typically 10 nM GST-hK-RasG12C and 2 nM antiGST-Eu(K) (Cisbio, France). A SOS working solution was prepared in assay buffer containing typically 20 nM His-hSOS1 and 10 nM anti-6His-XL665 (Cisbio, France). An inhibitor control solution was prepared in assay buffer containing 10 nM anti-6His-XL665 without hSOS1.

Fifty nl of a 100-fold concentrated solution of the test compound in DMSO were transferred into a black microtiter test plate (384 or 1536, Greiner Bio-One, Germany). For this, either a Hummingbird liquid handler (Digilab, MA, USA) or an Echo acoustic system (Labcyte, CA, USA) was used.

All steps of the assay were performed at 20° C. A volume of 2.5 μl of the Ras working solution was added to all wells of the test plate using a Multidrop dispenser (Thermo Labsystems). After 2 min preincubation, 2.5 μl of the SOS working solution were added to all wells except for those wells at the side of the test plate that were subsequently filled with 2.5 μl of the inhibitor control solution. After 60 min incubation the fluorescence was measured with a Pherastar (BMG, Germany) using the HTRF module (excitation 337 nm, emission 1: 620 nm, emission 2: 665 nm).

The ratiometric data (emission 2 divided by emission 1) were normalized using the controls (DMSO=0% inhibition, inhibition control wells with inhibitor control solution=100% inhibition). Compounds were tested in duplicates at up to 11 concentrations (for example 20 μM, 5.7 μM, 1.6 μM, 0.47 μM, 0.13 μM, 38 nM, 11 nM, 3.1 nM, 0.89 nM, 0.25 nM and 0,073 nM). IC50 values were calculated by 4-Parameter fitting using a commercial software package (Genedata Screener, Switzerland).

Biochemical Assay 2: K-Ras^{G12C} Activation Assay by hSOS1 at High GTP Concentration

This assay quantifies human SOS1-mediated nucleotide exchange of K-Ras^{G12C} preloaded with a fluorescent GTP-analog and in presence of an excess of free GTP. Loaded hK-Ras^{G12C} generates a high HTRF-signal by energy transfer from antiGST-Terbium (FRET donor) bound to K-Ras to the loaded fluorescent GDP analog (FRET-acceptor). SOS1 activity exchanges the fluorescent GDP for non-fluorescent GTP and therefore leads to a reduction of the HTRF signal.

The fluorescent GDP-analog EDA-GDP-Dy647P1 (2′/3′-O-(2-Aminoethyl-carbamoyl)-guanosine-5′-diphosphate labelled with Dy647P1 (Dyomics GmbH, Germany)) was synthesized by Jena Biosciences GmbH (Germany) and supplied as a 1 mM aqueous solution.

The expression and purification of N-terminal GST-tagged human K-RasG120 and N-terminal His-tagged human SOS1 is described below. Concentrations of protein batches used were optimized to be within the linear range of the HTRF signal.

Preparation of GST-tagged hK-Ras^{Gl2C} loaded with fluorescent nucleotide was performed as follows: incubation of 11.5 μM hK-Ras^{Gl2C} with 5-fold excess GDP-Dy647 nucleotide (54 μM) in 500 μl NLS-buffer (RAS activation Kit Jena Bioscience, Kat. #PR-950) for 10 min at 37° C. Addition of 20 μl 1 M MgCl₂ (Sigma) to final 40 mM and store on ice. Purification into buffer (10 mM HEPES pH 7.4 (Applichem), 150 mM NaCl (Sigma), 5 mM MgCl₂ (Sigma)) by use of a PD-Minitrap desalting column (GE Healthcare). Concentration of 1 ml purified hK-Ras-GDP-Dy647 is approx. 4-5 μM.

The assay buffer contained 10 mM HEPES pH 7.4 (Applichem), 150 mM NaCl (Sigma), 5 mM MgCl₂ (Sigma), 1 mM DTT (Thermofisher), 0.05% BSA Fraction V, pH 7.0, (ICN Biomedicals), 0.0025% (v/v) Igepal (Sigma).

A Ras working solution was prepared in assay buffer containing typically 80 nM loaded GST-hK-RasG12C-EDA-GDP-Dy647P1 and 2 nM antiGST-Tb (Cisbio, France). A hSOS1 working solution was prepared in assay buffer containing typically 8 nM His-hSOS1 and 100 μM GTP (Jena Bioscience, Germany). An inhibitor control solution was prepared in assay buffer containing the same concentration of hSOS1 without GTP. Alternatively, the inhibitor control solution was prepared by supplementing the hSOS1 working solution with 20 μM of 6,7-dimethoxy-N-[(1R)-1-(1-naphthyl)ethyl]quinazolin-4-amine which was used to calibrate the assay.

Fifty nl of a 100-fold concentrated solution of the test compound in DMSO were transferred into a black microtiter test plate (384 or 1536, Greiner Bio-One, Germany). For this, either a Hummingbird liquid handler (Digilab, MA, USA) or an Echo acoustic system (Labcyte, CA, USA) was used.

All steps of the assay were performed at 20° C. A volume of 2.5 μl of the Ras working solution was added to all wells of the test plate using a Multidrop dispenser (Thermo Labsystems). After 2 min preincubation, 2.5 μl of the hSOS1 working solution were added to all wells except for those wells at the side of the test plate that were subsequently filled with 2.5 μl of the inhibitor control solution. After 20 min incubation the fluorescence was measured with a Pherastar (BMG, Germany) using the HTRF module (excitation 337 nm, emission 1: 620 nm, emission 2: 665 nm).

The ratiometric data (emission 2 divided by emission 1) were normalized using the controls (DMSO=0% inhibition, inhibition control wells with inhibitor control solution=100% inhibition). Compounds were tested in duplicates at up to 11 concentrations (for example 20 μM, 5.7 μM, 1.6 μM, 0.47 μM, 0.13 μM, 38 nM, 11 nM, 3.1 nM, 0.89 nM, 0.25 nM and 0,073 nM). IC50 values were calculated by 4-Parameter fitting using a commercial software package (Genedata Screener, Switzerland).

Biochemical assay 3: K-Ras^{G12C} Activation Assay by hSOS1

K-Ras is a small GTPase that can bind GDP and GTP. The guanine nucleotide exchange factor SOS catalyzes the activation of K-Ras by promoting the exchange of GDP to GTP. SOS binds to K-Ras-GDP thereby opening the GDP-binding pocket to facilitate GDP release. Rebinding of excess nucleotide leads to dissociation of the K-Ras-SOS intermediate complex leaving K-Ras loaded with the nucleotide.

This assay quantifies human SOS1-mediated loading of human K-Ras^{G12C}-GDP with a fluorescent GTP-analog. Detection of successful loading is achieved by measuring homogenous time-resolved fluorescence resonance energy transfer (HTRF) from antiGST-Terbium (FRET donor) bound to GST-K-RasG12C to the loaded fluorescent GTP analog (FRET-acceptor).

The fluorescent GTP-analog EDA-GTP-Dy647P1 (2′/3′-O-(2-Aminoethyl-carbamoyl)-guanosine-5′-triphosphate labelled with Dy647P1 (Dyomics GmbH, Germany)) was synthesized by Jena Biosciences GmbH (Germany) and supplied as a 1 mM aqueous solution.

The assay buffer contained 10 mM HEPES pH 7.4 (Applichem), 150 mM NaCl (Sigma), 5 mM MgCl₂ (Sigma), 1 mM DTT (Thermofisher), 0.05% BSA Fraction V, pH 7.0, (ICN Biomedicals), 0.0025% (v/v) Igepal (Sigma).

The expression and purification of N-terminal GST-tagged human K-RasG120 and N-terminal His-tagged human SOS1 is described below. Concentrations of protein batches used were optimized to be within the linear range of the HTRF signal. A Ras working solution was prepared in assay buffer containing typically 100 nM GST-hK-RasG12C and 2 nM antiGST-Tb (Cisbio, France). A hSOS1 working solution was prepared in assay buffer containing typically 20 nM hSOS1 and 200 nM EDA-GTP-Dy647P1. An inhibitor control solution was prepared in assay buffer containing 200 nM EDA-GTP-Dy647P1 without hSOS1.

Fifty nl of a 100-fold concentrated solution of the test compound in DMSO were transferred into a black microtiter test plate (384 or 1536, Greiner Bio-One, Germany). For this, either a Hummingbird liquid handler (Digilab, MA, USA) or an Echo acoustic system (Labcyte, CA, USA) was used.

All steps of the assay were performed at 20° C. A volume of 2.5 μl of the Ras working solution was added to all wells of the test plate using a Multidrop dispenser (Thermo Labsystems). After 10 min preincubation, 2.5 μl of the hSOS1 working solution were added to all wells except for those wells at the side of the test plate that were subsequently filled with 2.5 μl of the inhibitor control solution. After 30 min incubation the fluorescence was measured with a Pherastar (BMG, Germany) using the HTRF module (excitation 337 nm, emission 1: 620 nm, emission 2: 665 nm).

The ratiometric data (emission 2 divided by emission 1) were normalized using the controls (DMSO=0% inhibition, inhibition control wells with inhibitor control solution=100% inhibition). Compounds were tested in duplicates at up to 11 concentrations (for example 20 μM, 5.7 μM, 1.6 μM, 0.47 μM, 0.13 μM, 38 nM, 11 nM, 3.1 nM, 0.89 nM, 0.25 nM and 0,073 nM). IC50 values were calculated by 4-Parameter fitting using a commercial software package (Genedata Screener, Switzerland).

EGFR Kinase Assay

EGFR inhibitory activity of compounds of the present invention was quantified employing the TR-FRET based EGFR assay as described in the following paragraphs.

Epidermal Growth Factor Receptor (EGFR) affinity purified from human carcinoma A431 cells (Sigma-Aldrich, #E3641) was used as kinase. As substrate for the kinase reaction the biotinylated peptide biotin-Ahx-AEEEEYFELVAKKK (SEQ ID NO 5) (C-terminus in amid form) was used which can be purchased e.g. form the company Biosyntan GmbH (Berlin-Buch, Germany).

For the assay 50 nL of a 100 fold concentrated solution of the test compound in DMSO was pipetted into a black low volume 384 well microtiter plate (Greiner Bio-One, Frickenhausen, Germany), 2 μL of a solution of EGFR in aqueous assay buffer [50 mM Hepes/HCl pH 7.0, 1 mM MgCl₂, 5 mM MnCl₂, 0.5 mM activated sodium ortho-vanadate, 0.005% (v/v) Tween-20]were added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 16.7 μM=>final conc. in the 5 μL assay volume is 10 μM) and substrate (1.67 μM=>final conc. in the 5 μL assay volume is 1 μM) in assay buffer and the resulting mixture was incubated for a reaction time of 20 min at 22° C. The concentration of EGFR was adjusted depending of the activity of the enzyme lot and was chosen appropriate to have the assay in the linear range, typical concentration were about 3 U/ml. The reaction was stopped by the addition of 5 μl of a solution of HTRF detection reagents (0.1 μM streptavidine-XL665 [Cis Biointernational] and 1 nM PT66-Tb-Cryptate, an terbium-cryptate labelled anti-phospho-tyrosine antibody from Cis Biointernational [instead of the PT66-Tb-cryptate PT66-Eu-Chelate from Perkin Elmer can also be used]) in an aqueous EDTA-solution (80 mM EDTA, 0.2% (w/v) bovine serum albumin in 50 mM HEPES pH 7.5).

The resulting mixture was incubated 1 h at 22° C. to allow the binding of the biotinylated phosphorylated peptide to the streptavidine-XL665 and the PT66-Eu-Chelate. Subsequently the amount of phosphorylated substrate was evaluated by measurement of the resonance energy transfer from the PT66-Tb-Cryptate to the streptavidine-XL665. Therefore, the fluorescence emissions at 620 nm and 665 nm after excitation at 337 nm were measured in a HTRF reader, e.g. a Pherastar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.072 nM (e.g. 20 μM, 5.7 μM, 1.6 μM, 0.47 μM, 0.13 μM, 38 nM, 11 nM, 3.1 nM, 0.89 nM, 0.25 nM and 0.072 nM, the dilution series prepared separately before the assay on the level of the 100 fold concentrated solutions in DMSO by serial dilutions, the exact concentrations may vary depending on the pipettor used) in duplicate values for each concentration and IC50 values were calculated by a 4 parameter fit.

Biochemical Assay 4: K-Ras^{G12C} Activation Assay by hSOS2

This assay quantifies human SOS2-mediated loading of K-Ras^{G12C}-GDP with a fluorescent GTP-analog. Detection of successful loading is achieved by measuring homogenous time-resolved fluorescence resonance energy transfer (HTRF) from antiGST-Terbium (FRET donor) bound to GST-hK-RasG120 to the loaded fluorescent GTP analog (FRET-acceptor).

The fluorescent GTP-analog EDA-GTP-Dy647P1 (2′/3′-O-(2-Aminoethyl-carbamoyl)-guanosine-5′-triphosphate labelled with Dy647P1 (Dyomics GmbH, Germany)) was synthesized by Jena Biosciences GmbH (Germany) and supplied as a 1 mM aqueous solution.

The assay buffer contained 10 mM HEPES pH 7.4 (Applichem), 150 mM NaCl (Sigma), 5 mM MgCl₂ (Sigma), 1 mM DTT (Thermofisher), 0.05% BSA Fraction V, pH 7.0, (ICN Biomedicals), 0.0025% (v/v) Igepal (Sigma).

The expression and purification of N-terminal GST-tagged human K-RasG120 and N-terminal His-tagged human SOS1 is described below. Concentrations of protein batches used were optimized to be within the linear range of the HTRF signal. A Ras working solution was prepared in assay buffer containing typically 100 nM GST-hK-RasG12C and 2 nM antiGST-Tb (Cisbio, France). A hSOS2 working solution was prepared in assay buffer containing typically 20 nM hSOS2 and 200 nM EDA-GTP-Dy647P1. An inhibitor control solution was prepared in assay buffer containing 200 nM EDA-GTP-Dy647P1 without hSOS2.

Fifty nl of a 100-fold concentrated solution of the test compound in DMSO were transferred into a black microtiter test plate (384 or 1536, Greiner Bio-One, Germany). For this, either a Hummingbird liquid handler (Digilab, MA, USA) or an Echo acoustic system (Labcyte, CA, USA) was used.

All steps of the assay were performed at 20° C. A volume of 2.5 μl of the Ras working solution was added to all wells of the test plate using a Multidrop dispenser (Thermo Labsystems). After 10 min preincubation, 2.5 μl of the hSOS2 working solution were added to all wells except for those wells at the side of the test plate that were subsequently filled with 2.5 μl of the inhibitor control solution. After 30 min incubation the fluorescence was measured with a Pherastar (BMG, Germany) using the HTRF module (excitation 337 nm, emission 1: 620 nm, emission 2: 665 nm).

The ratiometric data (emission 2 divided by emission 1) were normalized using the controls (DMSO=0% inhibition, inhibition control wells with inhibitor control solution=100% inhibition). Compounds were tested in duplicates at up to 11 concentrations (for example 20 μM, 5.7 μM, 1.6 μM, 0.47 μM, 0.13 μM, 38 nM, 11 nM, 3.1 nM, 0.89 nM, 0.25 nM and 0,073 nM). IC50 values were calculated by 4-Parameter fitting using a commercial software package (Genedata Screener, Switzerland).

KRAS Cellular Assays

3D-Softagar MiaPaca-2 (ATCC CRL-1420) and NCI-H1792 (ATCC CRL-5895)

Day 1: Softagar (Select Agar, Invitrogen, 3% in ddH2O autoclaved) is boiled and tempered at 48° C. Medium (MiaPaca-2: DMEM/Ham's F12; [Biochrom; #FG 4815, with stable Glutamine]10% FCS and 2.5% Horse Serum, H1792: RPM 1640; [Biochrom; #FG 1215, with stable Glutamine and 10% FCS]) is tempered to 37° C.; Agar (3%) is diluted 1:5 in medium (=0.6%) and 50 μl/well plated into 96 well plates (Corning, #3904), wait at room temperature until the agar is solid. 3% agar is diluted to 0.25% in medium (1:12 dilution) and tempered at 42° C. Cells are trypsinized, counted and tempered at 37° C.; cells (MiaPaCa-2: 125-150, NCI-H1792: 1000) are resuspended in 100 μl 0.25% Agar and plated. Wait at room temperature until the agar is solid. Overlay wells with 50 μl medium. Plate sister wells in separate plate for time zero determination. All plates are incubated overnight 37° C. and 5% CO2.

Day 2: Measurement of time zero values: Add 40 μl Cell Titer 96 Aqueous Solution (Promega) per well, (light sensitive) and incubate in the dark at 37° C. and 5% CO2. Absorption is measured at 490 nm and reference wavelength 660 nm. DMSO-prediluted test compounds are added with HP Dispenser to a final DMSO concentration of 0.3%.

Day 10: Measurement of test compound and control treated wells with Cell Titer 96 AQueous according to time zero. The IC50 values were determined using the four parameter fit.

Active RAS in Calu-1 Cells (CLS 300141)

40.000 Calu-1 cells are seeded in 96 well plate (NUNC161093) for 48h at 37° C./5% CO2 (10% FBS (S0615), DMEM/Ham's F-12 (Biochrom; #FG 4815), 2 mM L-Glutamine). After that, medium is changed to FBS-free medium and the cells were incubated for further 24h at 37° C./5% CO2. Cells are treated with varying concentrations of DMSO-prediluted test compounds (final 0.1%) for 30 min at 37° C./5% CO2. Supernatant with test compounds is discarded and, after that, treated cells are stimulated with 100 ng/ml EGF (Sigma #E9644, diluted in serum free medium) for 3 minutes. Cells were treated with lysis buffer and all next steps were performed on ice according to the supplier's manual of G-LISA Kit (Cytoskeleton BK131, Ras Activation Assay). Finally, the content of active Ras is measured by detecting the absorbance at 490 nm (Tecan Sunrise). The value of EGF-stimulated cells is set as 100%, whereas the value of untreated cells is set as 0%. The IC50 values were determined using the four parameter fit.

P-EGR Assay (In-Cell Western) in Hela Cells (ATCC CCL-2)

After stimulation with EGF, the EGF receptor autophosphorylates at Y1173. In-cell Western assay simultaneously detect two targets at 700 and 800 nm using two spectrally distinct near-infrared dyes. With a specific antibody, phosphorylated EGFR can be quantified and the samples can be normalized with total EGFR antibody parallel.

25000 Hela cells are seeded in 96 well plate (NUNC161093) for 24 h at 37° C./5%002 (10% FBS (S0615), DMEM/Ham's F-12 (Biochrom; #FG 4815), 2 mM L-Glutamine). After that, medium is changed to FBS-free medium and the cells were incubated for further 24h at 37° C./5%002. Cells are treated with varying concentrations of DMSO-prediluted test compounds (final 0.1%) for 30 minutes and finally with 100 ng/ml EGF (Sigma #E9644, diluted in serum free medium) for 2 minutes.

Cells are treated according the manual of EGFR Near Infrared In-Cell ELISA Kit (Pierce #62210). If not specified, all buffers and antibodies are part of this kit.

Cells are fixed with 4% formaldehyde, washed twice with 100 μl per well with TRIS-buffered saline with Surfact-Amps 20, permeabilized with 100 μl TRIS-buffered saline with Surfact-Amps X-100, wash again with 100 μl TRIS-buffered saline, and finally 200 μl blocking buffer are added for 60 minutes at room temperature. Fixed and washed cells are incubated with primary antibody mix (P-EGFR; EGFR) overnight at 2-8° C. After washing with 100 μl TRIS-buffered saline with Surfact-Amps 20, secondary IRDye-labeled antibody mix (DyLight 800 Goat Anti-Rabbit IgG, Pierce SA5-35571; DyLight 680 Goat Anti-Mouse IgG, Pierce 35518) is added for 1h at room temperature and washed again. Plates are scanned with LiCor Odyssey Infrared Imager at 800 nm for P-EGFR and at 700 nm for total EGFR. The quotient of 800 nm and 700 nm for EGF only treated cells is set as 100% and the quotient of 800 nm and 700 nm of untreated cells is set as 0%. The 1050 values were determined using the four parameter fit.

TABLE 1
IC₅₀ values of the compounds of examples 1 to 458 in in vitro assays 1 to
3 and the EGFR-assay (“n.d.” means “not determined”)

| | Assay 1 | Assay 2 | Assay 3 | |
|---|---|---|---|---|
| | (KRAS-SOS | (KRAS-Activation | (KRAS-Activation | |
| | Interaction) | by SOS high GTP) | by SOS no GTP) | EGFR |
| | activity (expressed | activity (expressed | activity (expressed | kinase |
| Example | as IC50, or as % | as IC50, or as % | as IC50, or as % | inhibition |
| No. | inhibition at 20 μM) | inhibition at 20 μM) | inhibition at 20 μM) | IC50 |
| Reference | 3.04E−07 | 2.63E−07 | 6.93E−07 | 5.53E−07 |
| compound | | | | |
| above | | | | |
| 1 | 1.33E−07 | 3.03E−07 | 3.70E−07 | >20 μM |
| 2 | 1.27E−07 | 2.07E−07 | 5.37E−07 | 1.63E−05 |
| 3 | 1.13E−07 | 1.15E−07 | 2.31E−07 | >20 μM |
| 4 | 1.61E−07 | 2.43E−07 | 4.79E−07 | >20 μM |
| 5 | 1.71E−06 | 1.30E−06 | 5.06E−06 | >20 μM |
| 6 | 2.28E−06 | 1.86E−06 | 4.98E−06 | >20 μM |
| 7 | 44% | 18% | 33% | >20 μM |
| 8 | 1.82E−05 | 1.98E−05 | 14% | >20 μM |
| 9 | 1.52E−06 | 1.37E−06 | 5.96E−06 | >20 μM |
| 10 | 1.18E−06 | 1.27E−06 | 6.14E−06 | >20 μM |
| 11 | 2.03E−07 | 2.21E−07 | 5.40E−07 | >20 μM |
| 12 | 3.35E−07 | 5.78E−07 | 1.29E−06 | >20 μM |
| 13 | 2.02E−06 | 2.65E−06 | 7.73E−06 | >20 μM |
| 14 | 6.66E−07 | 6.08E−07 | 1.83E−06 | >20 μM |
| 15 | 1.26E−07 | 9.11E−08 | 3.24E−07 | >20 μM |
| 16 | 3.25E−06 | 3.02E−06 | 8.17E−06 | >20 μM |
| 17 | 2.70E−07 | 2.97E−07 | 7.12E−07 | >20 μM |
| 18 | 6.50E−06 | 7.68E−06 | 1.05E−05 | >20 μM |
| 19 | 7.78E−06 | 6.55E−06 | 44% | >20 μM |
| 20 | 8.42E−06 | 8.20E−06 | 41% | >20 μM |
| 21 | 3.16E−06 | 3.23E−06 | 1.11E−05 | >20 μM |
| 22 | 1.77E−07 | 2.19E−07 | 5.89E−07 | >20 μM |
| 23 | 2.72E−06 | 1.85E−06 | 8.62E−06 | >20 μM |
| 24 | 7.01E−07 | 1.22E−06 | 2.45E−06 | >20 μM |
| 25 | 5.41E−07 | 8.50E−07 | 1.50E−06 | >20 μM |
| 26 | 9.55E−06 | 52% | 6.96E−06 | >20 μM |
| 27 | 3.68E−07 | 4.02E−07 | 4.98E−07 | >20 μM |
| 28 | 40% | 8% | 46% | >20 μM |
| 29 | 1.02E−05 | 1.09E−05 | 44% | >20 μM |
| 30 | 1.34E−06 | 1.10E−06 | 2.91E−06 | >20 μM |
| 31 | 8.59E−06 | 9.51E−06 | 49% | >20 μM |
| 32 | 1.07E−06 | 1.40E−06 | 2.59E−06 | >20 μM |
| 33 | 53% | 24% | 9% | >20 μM |
| 34 | 4.23E−06 | 3.71E−06 | 1.68E−05 | >20 μM |
| 35 | 4.72E−06 | 4.78E−06 | 48% | >20 μM |
| 36 | 5.42E−07 | 1.00E−06 | 6.96E−07 | n.d. |
| 37 | 1.54E−06 | 1.85E−06 | 2.69E−06 | n.d. |
| 38 | n.d. | n.d. | n.d. | n.d. |
| 39 | 1.46E−06 | 1.83E−06 | 3.05E−06 | n.d. |
| 40 | 3.85E−06 | 1.75E−06 | 4.55E−06 | n.d. |
| 41 | 9.72E−07 | 1.40E−06 | 1.67E−06 | n.d. |
| 42 | 4.27E−06 | 5.58E−06 | 48% | n.d. |
| 43 | 30% | 14% | 16% | n.d. |
| 44 | 55% | 47% | 25% | n.d. |
| 45 | 1.51E−06 | 2.17E−06 | 1.10E−06 | n.d. |
| 46 | 2.37E−06 | 3.82E−06 | 4.10E−06 | n.d. |
| 47 | 40% | 22% | 17% | n.d. |
| 48 | 2.12E−06 | 3.27E−06 | 4.52E−06 | n.d. |
| 49 | 1.96E−05 | 35% | 36% | n.d. |
| 50 | 9.85E−06 | 2.00E−05 | 23% | n.d. |
| 51 | 5.63E−06 | 5.90E−06 | 52% | n.d. |
| 52 | 4.29E−06 | 5.46E−06 | 43% | n.d. |
| 53 | 6.69E−07 | 1.28E−06 | 1.08E−06 | n.d. |
| 54 | 4.19E−06 | 4.13E−06 | 4.07E−06 | n.d. |
| 55 | 6.74E−07 | 6.29E−07 | 7.24E−07 | n.d. |
| 56 | 1.47E−06 | 1.79E−06 | 2.56E−06 | n.d. |
| 57 | 4.14E−07 | 4.01E−07 | 6.29E−07 | n.d. |
| 58 | 1.77E−06 | 1.64E−06 | 3.11E−06 | n.d. |
| 59 | 1.59E−06 | 7.70E−07 | 9.65E−07 | n.d. |
| 60 | 5.85E−07 | 4.48E−07 | 1.04E−06 | n.d. |
| 61 | 3.74E−07 | 7.17E−07 | 8.95E−07 | n.d. |
| 62 | 1.26E−06 | 2.77E−06 | 2.63E−06 | n.d. |
| 63 | 1.13E−06 | 1.80E−06 | 1.88E−06 | n.d. |
| 64 | 1.40E−06 | 1.96E−06 | 2.21E−06 | n.d. |
| 65 | 5.76E−06 | 8.34E−06 | 1.35E−05 | n.d. |
| 66 | 3.46E−06 | 4.55E−06 | 6.60E−06 | n.d. |
| 67 | 9.98E−06 | 1.96E−05 | 54% | n.d. |
| 68 | 1.37E−07 | 4.86E−07 | 4.71E−07 | 6.92E−06 |
| 69 | 3.92E−06 | 5.41E−06 | 9.07E−06 | n.d. |
| 70 | 6.82E−06 | 8.62E−06 | 47% | n.d. |
| 71 | 3.93E−07 | 6.43E−07 | 6.80E−07 | n.d. |
| 72 | 7.37E−07 | 6.66E−07 | 8.82E−07 | n.d. |
| 73 | 4.27E−07 | 5.54E−07 | 4.95E−07 | n.d. |
| 74 | 1.49E−07 | 2.23E−07 | 2.61E−07 | n.d. |
| 75 | 5.88E−07 | 1.15E−06 | 1.01E−06 | n.d. |
| 76 | 4.71E−07 | 1.19E−06 | 9.52E−07 | n.d. |
| 77 | 7.86E−06 | 9.63E−06 | 1.99E−05 | n.d. |
| 78 | 8.55E−06 | 45% | 31% | n.d. |
| 79 | 1.55E−06 | 9.37E−07 | 1.65E−06 | n.d. |
| 80 | 7.21E−07 | 7.11E−07 | 1.12E−06 | n.d. |
| 81 | 42% | 33% | 24% | n.d. |
| 82 | 5.98E−07 | 8.57E−07 | 1.00E−06 | n.d. |
| 83 | 8.50E−06 | 7.85E−06 | 1.74E−05 | n.d. |
| 84 | 5.33E−06 | 7.20E−06 | 1.10E−05 | n.d. |
| 85 | 6.87E−07 | 7.56E−07 | 1.26E−06 | n.d. |
| 86 | 5.81E−07 | 9.35E−07 | 6.93E−07 | n.d. |
| 87 | 4.42E−06 | 3.46E−06 | 7.42E−06 | n.d. |
| 88 | 3.56E−06 | 2.56E−06 | 6.18E−06 | n.d. |
| 89 | 1.68E−06 | 2.68E−06 | 4.26E−06 | n.d. |
| 90 | 1.10E−05 | 46% | 33% | n.d. |
| 91 | 1.50E−05 | 36% | 38% | n.d. |
| 92 | 4.70E−06 | 3.67E−06 | 9.26E−06 | n.d. |
| 93 | 1.85E−06 | 1.58E−06 | 2.89E−06 | n.d. |
| 94 | 2.42E−06 | 3.14E−06 | 4.28E−06 | n.d. |
| 95 | 8.01E−07 | 4.73E−07 | 1.08E−06 | n.d. |
| 96 | 1.04E−06 | 1.09E−06 | 1.39E−06 | n.d. |
| 97 | 39% | 28% | 38% | n.d. |
| 98 | 1.61E−05 | 43% | 24% | n.d. |
| 99 | 2.18E−06 | 1.42E−06 | 2.63E−06 | n.d. |
| 100 | 5.35E−06 | 4.51E−06 | 8.52E−06 | n.d. |
| 101 | 7.15E−07 | 8.97E−07 | 1.80E−06 | n.d. |
| 102 | 6.38E−06 | 3.56E−06 | 1.15E−05 | n.d. |
| 103 | 1.44E−06 | 2.48E−06 | 2.74E−06 | n.d. |
| 104 | 1.82E−06 | 1.90E−06 | 2.52E−06 | n.d. |
| 105 | 2.40E−06 | 1.39E−06 | 2.77E−06 | n.d. |
| 106 | 1.41E−06 | 1.53E−06 | 2.05E−06 | n.d. |
| 107 | 3.19E−07 | 4.67E−07 | 4.38E−07 | n.d. |
| 108 | 8.21E−07 | 9.01E−07 | 1.23E−06 | n.d. |
| 109 | 1.00E−06 | 3.92E−07 | 1.41E−06 | n.d. |
| 110 | 1.04E−06 | 9.08E−07 | 1.19E−06 | n.d. |
| 111 | 2.10E−06 | 1.67E−06 | 3.34E−06 | n.d. |
| 112 | 6.96E−07 | 9.48E−07 | 1.12E−06 | n.d. |
| 113 | 1.35E−06 | 8.39E−07 | 2.10E−06 | n.d. |
| 114 | 5.28E−06 | 2.39E−06 | 9.21E−06 | n.d. |
| 115 | 8.65E−07 | 8.02E−07 | 1.57E−06 | n.d. |
| 116 | 1.08E−06 | 1.15E−06 | 1.72E−06 | n.d. |
| 117 | 1.65E−06 | 1.35E−06 | 3.08E−06 | n.d. |
| 118 | 5.29E−07 | 4.81E−07 | 7.74E−07 | n.d. |
| 119 | 8.58E−07 | 1.28E−06 | 1.48E−06 | n.d. |
| 120 | 1.59E−06 | 2.02E−06 | 3.53E−06 | n.d. |
| 121 | 4.52E−07 | 6.79E−07 | 7.91E−07 | n.d. |
| 122 | 46% | 17% | 21% | n.d. |
| 123 | 52% | 1.31E−05 | 42% | n.d. |
| 124 | 1.83E−05 | 9.30E−06 | 37% | n.d. |
| 125 | 2.03E−06 | 1.09E−06 | 2.04E−06 | n.d. |
| 126 | 52% | 31% | 11% | n.d. |
| 127 | 1.78E−05 | 44% | 42% | n.d. |
| 128 | 1.39E−05 | 1.69E−05 | 16% | n.d. |
| 129 | 1.48E−06 | 2.38E−06 | 3.45E−06 | n.d. |
| 130 | 3.43E−06 | 4.31E−06 | 1.17E−05 | n.d. |
| 131 | 2.23E−06 | 3.37E−06 | 5.94E−06 | n.d. |
| 132 | 6.58E−06 | 8.05E−06 | 46% | n.d. |
| 133 | 4.30E−06 | 4.03E−06 | 5.67E−06 | n.d. |
| 134 | 43% | 23% | 27% | n.d. |
| 135 | 28% | 5% | 13% | n.d. |
| 136 | 37% | 20% | 18% | n.d. |
| 137 | 48% | 36% | 32% | n.d. |
| 138 | 5.57E−06 | 7.43E−06 | 40% | n.d. |
| 139 | 4.70E−06 | 6.13E−06 | 41% | n.d. |
| 140 | 5.33E−06 | 5.26E−06 | 33% | n.d. |
| 141 | 5.63E−06 | 25% | 1.11E−05 | n.d. |
| 142 | 1.01E−06 | 1.81E−06 | 1.02E−06 | n.d. |
| 143 | 7.64E−06 | 8.76E−06 | 1.78E−05 | n.d. |
| 144 | 30% | 12% | 19% | n.d. |
| 145 | 40% | 18% | 11% | n.d. |
| 146 | 9.11E−07 | 1.50E−06 | 1.48E−06 | n.d. |
| 147 | 3.97E−07 | 7.67E−07 | 6.58E−07 | n.d. |
| 148 | 1.41E−06 | 3.40E−06 | 3.06E−06 | n.d. |
| 149 | 2.36E−06 | 4.30E−06 | 17% | n.d. |
| 150 | 7.36E−06 | 1.32E−05 | 1.32E−05 | n.d. |
| 151 | 1.04E−05 | 51% | 1.33E−05 | n.d. |
| 152 | 5.48E−06 | 7.33E−06 | 1.11E−05 | n.d. |
| 153 | 2.35E−06 | 2.73E−06 | 4.10E−06 | n.d. |
| 154 | 7.82E−06 | 8.78E−06 | 1.29E−05 | n.d. |
| 155 | 9.92E−07 | 1.47E−06 | 1.64E−06 | n.d. |
| 156 | 4.12E−06 | 5.76E−06 | 1.08E−05 | n.d. |
| 157 | 44% | 19% | 12% | n.d. |
| 158 | 29% | 4% | 10% | n.d. |
| 159 | 43% | 27% | 35% | n.d. |
| 160 | 1.61E−05 | 33% | 13% | n.d. |
| 161 | 4.17E−06 | 3.97E−06 | 7.00E−06 | n.d. |
| 162 | 8.76E−06 | 1.16E−05 | 1.60E−05 | n.d. |
| 163 | 1.36E−05 | 42% | 35% | n.d. |
| 164 | 41% | 19% | 22% | n.d. |
| 165 | 1.06E−05 | 1.64E−05 | 32% | n.d. |
| 166 | 1.80E−06 | 3.68E−06 | 4.99E−06 | n.d. |
| 167 | 1.69E−05 | 40% | 37% | n.d. |
| 168 | 1.22E−05 | 38% | 32% | n.d. |
| 169 | 2.16E−06 | 3.70E−06 | 4.53E−06 | n.d. |
| 170 | 45% | 7% | 15% | n.d. |
| 171 | 5.59E−06 | 3.37E−06 | 7.18E−06 | n.d. |
| 172 | 4.07E−06 | 7.33E−06 | 9.19E−06 | n.d. |
| 173 | 6.88E−06 | 8.05E−06 | 48% | n.d. |
| 174 | 6.92E−06 | 9.28E−06 | 1.83E−05 | n.d. |
| 175 | 1.76E−05 | 50% | 44% | n.d. |
| 176 | 3.49E−06 | 5.82E−06 | 42% | n.d. |
| 177 | 5.77E−06 | 6.70E−06 | 49% | n.d. |
| 178 | 1.43E−06 | 5.11E−06 | 7.12E−06 | >20 μM |
| 179 | 9.04E−08 | 2.45E−07 | 2.14E−07 | >20 μM |
| 180 | 2.41E−07 | 9.21E−07 | 8.20E−07 | >20 μM |
| 181 | 3.50E−06 | 6.51E−06 | 45% | >20 μM |
| 182 | 5.10E−08 | 1.23E−07 | 1.21E−07 | >20 μM |
| 183 | 2.11E−06 | 4.20E−06 | 9.14E−06 | >20 μM |
| 184 | 4.16E−08 | 1.06E−07 | 1.22E−07 | >20 μM |
| 185 | 7.88E−08 | 3.44E−07 | 3.23E−07 | >20 μM |
| 186 | 9.94E−06 | 3.31E−06 | 22% | >20 μM |
| 187 | 4.76E−07 | 5.62E−07 | 1.26E−06 | >20 μM |
| 188 | 1.57E−05 | 1.67E−05 | 38% | >20 μM |
| 189 | 9.98E−07 | 1.17E−06 | 2.76E−06 | >20 μM |
| 190 | 1.75E−07 | 1.53E−07 | 5.53E−07 | >20 μM |
| 191 | 1.74E−07 | 2.45E−07 | 3.82E−07 | >20 μM |
| 192 | 4.59E−06 | 4.09E−06 | 1.42E−05 | >20 μM |
| 193 | 4.16E−07 | 4.05E−07 | 8.20E−07 | >20 μM |
| 194 | 4.93E−07 | 5.94E−07 | 2.43E−06 | >20 μM |
| 195 | 3.00E−07 | 6.35E−07 | 7.49E−07 | >20 μM |
| 196 | 2.42E−06 | 1.09E−06 | 6.10E−06 | >20 μM |
| 197 | 2.68E−07 | 3.03E−07 | 6.38E−07 | >20 μM |
| 198 | 2.91E−07 | 5.18E−07 | 1.09E−06 | >20 μM |
| 199 | 1.73E−06 | 1.42E−06 | 6.70E−06 | >20 μM |
| 200 | 1.39E−07 | 2.38E−07 | 4.04E−07 | >20 μM |
| 201 | 1.97E−05 | 20% | 50% | >20 μM |
| 202 | 1.42E−06 | 9.79E−07 | 5.32E−06 | >20 μM |
| 203 | 1.37E−06 | 1.38E−06 | 3.36E−06 | >20 μM |
| 204 | 2.29E−07 | 2.65E−07 | 6.40E−07 | >20 μM |
| 205 | 7.38E−08 | 8.72E−08 | 1.26E−07 | >20 μM |
| 206 | 4.81E−07 | 1.00E−06 | 1.75E−06 | >20 μM |
| 207 | 2.73E−06 | 2.27E−06 | 1.23E−05 | >20 μM |
| 208 | 5.97E−06 | n.d. | 47% | >20 μM |
| 209 | 1.08E−06 | n.d. | 4.95E−06 | >20 μM |
| 210 | 3.85E−08 | 1.09E−07 | 1.00E−07 | >20 μM |
| 211 | 1.33E−07 | 2.92E−07 | 2.33E−07 | >20 μM |
| 212 | 6.46E−07 | n.d. | 2.64E−06 | >20 μM |
| 213 | 6.76E−06 | 6.31E−06 | 42% | >20 μM |
| 214 | 3.33E−06 | 3.96E−06 | 1.36E−05 | >20 μM |
| 215 | 9.06E−07 | 1.16E−06 | 2.95E−06 | 2.57E−06 |
| 216 | 6.58E−06 | 1.70E−05 | 1.61E−05 | >20 μM |
| 217 | 8.80E−06 | 50% | 45% | >20 μM |
| 218 | 1.96E−06 | 1.37E−06 | 3.78E−06 | >20 μM |
| 219 | 4.15E−07 | 5.70E−07 | 9.08E−07 | >20 μM |
| 220 | 3.79E−07 | 3.81E−07 | 1.01E−06 | >20 μM |
| 221 | 3.06E−06 | 4.65E−06 | 4.81E−06 | >20 μM |
| 222 | 3.88E−06 | 4.44E−06 | 1.35E−05 | >20 μM |
| 223 | 2.11E−06 | 5.60E−06 | 7.18E−06 | >20 μM |
| 224 | 3.60E−06 | 2.55E−06 | 9.37E−06 | >20 μM |
| 225 | 1.54E−06 | 2.27E−06 | 4.68E−06 | >20 μM |
| 226 | 43% | 37% | 24.00% | >20 μM |
| 227 | 48% | 24% | 34% | >20 μM |
| 228 | 5.69E−07 | 6.47E−07 | 1.43E−06 | 3.41E−07 |
| 229 | 6.95E−06 | 7.82E−06 | 29% | >20 μM |
| 230 | 4.90E−06 | 6.69E−06 | 1.33E−05 | >20 μM |
| 231 | 56% | 53% | 24% | >20 μM |
| 232 | 9.01E−06 | 9.29E−06 | 4.04E−06 | >20 μM |
| 233 | 1.04E−05 | 2.00E−06 | 1.61E−05 | >20 μM |
| 234 | 7.56E−08 | 6.54E−08 | 9.21E−08 | >20 μM |
| 235 | 5.47E−06 | 4.72E−06 | 5.27E−06 | >20 μM |
| 236 | 37% | 3.82E−06 | 17% | >20 μM |
| 237 | 31% | 32% | 10% | >20 μM |
| 238 | 8.16E−08 | 2.17E−07 | 2.16E−07 | >20 μM |
| 239 | 7.07E−06 | 3.83E−06 | 1.43E−05 | >20 μM |
| 240 | 3.21E−06 | 3.19E−06 | 7.57E−06 | >20 μM |
| 241 | 1.12E−06 | 1.44E−06 | 1.31E−06 | >20 μM |
| 242 | 1.83E−06 | 1.50E−06 | 3.01E−06 | >20 μM |
| 243 | 2.52E−07 | 3.25E−07 | 4.18E−07 | >20 μM |
| 244 | 2.82E−07 | 4.01E−07 | 4.44E−07 | >20 μM |
| 245 | 5.92E−07 | 9.00E−07 | 9.18E−07 | >20 μM |
| 246 | 7.59E−06 | 4.21E−06 | 44% | >20 μM |
| 247 | 5.91E−07 | 8.67E−07 | 2.70E−06 | >20 μM |
| 248 | 1.49E−06 | 8.08E−06 | 8.43E−06 | >20 μM |
| 249 | 6.10E−06 | 5.31E−06 | 8.79E−06 | >20 μM |
| 250 | 5.00E−08 | 5.81E−08 | 7.04E−08 | >20 μM |
| 251 | 4.57E−06 | 5.84E−06 | 52% | >20 μM |
| 252 | 8.02E−08 | 7.41E−08 | 1.03E−07 | >20 μM |
| 253 | 1.31E−06 | 2.33E−06 | 5.80E−06 | >20 μM |
| 254 | 8.14E−07 | 1.59E−06 | 3.45E−06 | >20 μM |
| 255 | 2.48E−07 | 3.17E−07 | 3.90E−07 | >20 μM |
| 256 | 4.73E−07 | 8.28E−07 | 1.35E−06 | >20 μM |
| 257 | 3.63E−07 | 7.54E−07 | 8.52E−07 | >20 μM |
| 258 | 2.04E−07 | 1.90E−07 | 2.45E−07 | >20 μM |
| 259 | 4.02E−07 | 4.35E−07 | 6.50E−07 | >20 μM |
| 260 | 4.88E−06 | 2.04E−06 | 5.68E−06 | >20 μM |
| 261 | 8.39E−07 | 8.73E−07 | 1.10E−06 | >20 μM |
| 262 | 4.03E−08 | 4.40E−08 | 5.11E−08 | >20 μM |
| 263 | 2.37E−07 | 2.32E−07 | 3.24E−07 | >20 μM |
| 264 | 5.94E−06 | 1.67E−06 | 4.61E−06 | >20 μM |
| 265 | 2.77E−06 | 1.47E−06 | 1.11E−06 | >20 μM |
| 266 | 3.61E−06 | 1.70E−06 | 3.74E−06 | >20 μM |
| 267 | 3.39E−06 | 1.52E−06 | 1.78E−06 | >20 μM |
| 268 | 1.66E−07 | 1.51E−07 | 1.89E−07 | >20 μM |
| 269 | 1.62E−07 | 1.15E−07 | 1.35E−07 | >20 μM |
| 270 | 9.14E−08 | 8.22E−08 | 9.80E−08 | >20 μM |
| 271 | 6.05E−08 | 7.26E−08 | 7.59E−08 | >20 μM |
| 272 | 1.82E−06 | 1.50E−06 | 2.50E−06 | >20 μM |
| 273 | 2.32E−06 | 2.36E−06 | 3.63E−06 | >20 μM |
| 274 | 1.30E−07 | 1.41E−07 | 2.14E−07 | >20 μM |
| 275 | 2.80E−06 | 2.97E−06 | 6.37E−06 | >20 μM |
| 276 | 1.41E−06 | 1.66E−06 | 2.16E−06 | >20 μM |
| 277 | 2.14E−07 | 2.37E−07 | 2.63E−07 | >20 μM |
| 278 | 1.25E−05 | 3.71E−06 | 43% | >20 μM |
| 279 | 1.76E−07 | 1.68E−07 | 1.60E−07 | >20 μM |
| 280 | 1.48E−07 | 1.20E−07 | 1.35E−07 | >20 μM |
| 281 | 5.59E−07 | 4.47E−07 | 3.67E−07 | >20 μM |
| 282 | 2.57E−07 | 2.65E−07 | 2.12E−07 | >20 μM |
| 283 | 4.18E−07 | 3.71E−07 | 3.44E−07 | >20 μM |
| 284 | 1.06E−06 | 8.79E−07 | 8.89E−07 | >20 μM |
| 285 | 2.03E−07 | 1.58E−07 | 1.61E−07 | >20 μM |
| 286 | 1.67E−05 | 1.74E−05 | 36% | >20 μM |
| 287 | 5.88E−06 | 1.86E−06 | 5.76E−06 | >20 μM |
| 288 | 1.70E−07 | 1.40E−07 | 2.47E−07 | >20 μM |
| 289 | 2.11E−06 | 1.18E−06 | 2.73E−06 | >20 μM |
| 290 | 1.76E−07 | 1.16E−07 | 1.59E−07 | >20 μM |
| 291 | 1.12E−06 | 1.32E−06 | 2.23E−06 | >20 μM |
| 292 | 3.15E−08 | 3.87E−08 | 4.13E−08 | >20 μM |
| 293 | 2.75E−06 | 3.92E−06 | 8.20E−06 | >20 μM |
| 294 | 3.10E−06 | 3.93E−06 | 8.58E−06 | >20 μM |
| 295 | 9.80E−07 | 1.47E−06 | 2.78E−06 | >20 μM |
| 296 | 34% | 48% | 1.18E−05 | >20 μM |
| 297 | 4.60E−08 | 5.78E−08 | 6.06E−08 | >20 μM |
| 298 | 4.72E−08 | 4.93E−08 | 5.88E−08 | >20 μM |
| 299 | 9.48E−07 | 1.23E−06 | 1.88E−06 | >20 μM |
| 300 | 3.15E−06 | 3.07E−06 | 5.08E−06 | >20 μM |
| 301 | 7.99E−07 | 9.31E−07 | 1.93E−06 | >20 μM |
| 302 | 7.87E−07 | 1.26E−06 | 1.54E−06 | >20 μM |
| 303 | 7.24E−07 | 8.40E−07 | 8.80E−07 | >20 μM |
| 304 | 4.17E−07 | 4.69E−07 | 3.99E−07 | >20 μM |
| 305 | 6.16E−08 | 5.52E−08 | 5.84E−08 | >20 μM |
| 306 | 45% | 4.20E−06 | 7.94E−06 | >20 μM |
| 307 | 5.89E−07 | 5.93E−07 | 7.80E−07 | >20 μM |
| 308 | 2.60E−07 | 2.29E−07 | 2.20E−07 | >20 μM |
| 309 | 1.32E−06 | 6.90E−07 | 8.80E−07 | >20 μM |
| 310 | 5.82E−06 | 1.34E−06 | 5.76E−06 | >20 μM |
| 311 | 7.63E−07 | 5.13E−07 | 6.29E−07 | >20 μM |
| 312 | 6.82E−08 | 6.65E−08 | 7.32E−08 | >20 μM |
| 313 | 1.67E−07 | 1.62E−07 | 1.50E−07 | >20 μM |
| 314 | 1.04E−07 | 8.73E−08 | 9.01E−08 | >20 μM |
| 315 | 7.77E−07 | 9.67E−07 | 1.28E−06 | >20 μM |
| 316 | 2.71E−07 | 2.84E−07 | 3.08E−07 | >20 μM |
| 317 | 7.60E−08 | 6.27E−08 | 8.84E−08 | >20 μM |
| 318 | 1.71E−07 | 1.67E−07 | 2.21E−07 | >20 μM |
| 319 | 1.64E−07 | 1.19E−07 | 1.27E−07 | >20 μM |
| 320 | 2.26E−06 | 1.30E−06 | 3.49E−06 | >20 μM |
| 321 | 1.48E−05 | 6.24E−06 | 1.28E−05 | >20 μM |
| 322 | 4.08E−07 | 2.47E−07 | 2.68E−07 | >20 μM |
| 323 | 3.95E−08 | 7.29E−08 | 6.64E−08 | >20 μM |
| 324 | 1.29E−06 | 3.38E−06 | 6.84E−06 | >20 μM |
| 325 | 2.28E−07 | 4.43E−07 | 4.38E−07 | >20 μM |
| 326 | 1.29E−08 | 2.91E−08 | 2.77E−08 | >20 μM |
| 327 | 1.38E−06 | 1.94E−06 | 2.56E−06 | >20 μM |
| 328 | 1.42E−07 | 5.26E−07 | 6.69E−07 | >20 μM |
| 329 | 2.41E−06 | 6.47E−06 | 1.06E−05 | >20 μM |
| 330 | 2.27E−07 | 3.65E−07 | 5.47E−07 | >20 μM |
| 331 | 9.19E−07 | 9.92E−07 | 3.17E−06 | >20 μM |
| 332 | 2.25E−06 | 2.62E−06 | 1.14E−05 | >20 μM |
| 333 | 1.30E−05 | 6.20E−06 | 1.48E−05 | 1.64E−05 |
| 334 | 3.62E−07 | 7.73E−07 | 1.41E−06 | >20 μM |
| 335 | 1.45E−07 | 3.71E−07 | 5.64E−07 | >20 μM |
| 336 | 6.87E−07 | 1.26E−06 | 1.99E−06 | >20 μM |
| 337 | 3.44E−07 | 8.06E−07 | 1.35E−06 | >20 μM |
| 338 | 3.25E−07 | 8.38E−07 | 1.39E−06 | >20 μM |
| 339 | 8.15E−07 | 1.38E−06 | 1.99E−06 | >20 μM |
| 340 | 3.44E−07 | 9.09E−07 | 1.39E−06 | >20 μM |
| 341 | 2.83E−06 | 3.73E−06 | 1.22E−05 | >20 μM |
| 342 | 5.44E−07 | 9.93E−07 | 1.13E−06 | >20 μM |
| 343 | 1.68E−06 | 1.61E−06 | 4.33E−06 | >20 μM |
| 344 | 9.31E−07 | 1.19E−06 | 1.86E−06 | >20 μM |
| 345 | 1.46E−05 | 7.84E−06 | ! 2.94E−5 | >20 μM |
| 346 | 3.42E−07 | 3.95E−07 | 5.76E−07 | >20 μM |
| 347 | 1.56E−05 | 9.05E−06 | 48% | >20 μM |
| 348 | 6.93E−07 | 8.16E−07 | 7.53E−07 | >20 μM |
| 349 | 2.31E−07 | 6.49E−07 | 7.40E−07 | >20 μM |
| 350 | 6.88E−07 | 7.36E−07 | 8.50E−07 | >20 μM |
| 351 | 1.71E−06 | 1.20E−06 | 1.31E−06 | >20 μM |
| 352 | 1.60E−05 | 1.22E−05 | 1.82E−05 | >20 μM |
| 353 | 3.60E−07 | 4.20E−07 | 4.53E−07 | >20 μM |
| 354 | 3.13E−07 | 5.16E−07 | 6.77E−07 | >20 μM |
| 355 | 4.85E−07 | 5.71E−07 | 7.14E−07 | >20 μM |
| 356 | 6.55E−07 | 7.77E−07 | 9.33E−07 | >20 μM |
| 357 | 5.12E−08 | 4.88E−08 | 6.92E−08 | >20 μM |
| 358 | 1.44E−06 | 1.53E−06 | 2.45E−06 | >20 μM |
| 359 | 2.34E−07 | 2.00E−07 | 2.99E−07 | >20 μM |
| 360 | 1.95E−07 | 1.41E−07 | 1.90E−07 | >20 μM |
| 361 | 2.46E−06 | 4.05E−06 | 8.60E−06 | >20 μM |
| 362 | 26% | 27% | 30% | >20 μM |
| 363 | 1.24E−05 | 1.13E−05 | 1.72E−05 | >20 μM |
| 364 | 1.29E−07 | 1.58E−07 | 2.17E−07 | >20 μM |
| 365 | 4.69E−07 | 2.99E−07 | 3.53E−07 | >20 μM |
| 366 | 9.76E−08 | 9.69E−08 | 1.36E−07 | >20 μM |
| 367 | 7.16E−08 | 6.13E−08 | 9.90E−08 | >20 μM |
| 368 | 41% | 39% | 6% | >20 μM |
| 369 | 9.95E−08 | 1.52E−07 | 1.69E−07 | >20 μM |
| 370 | 2.70E−07 | 3.52E−07 | 3.63E−07 | >20 μM |
| 371 | 1.89E−06 | 2.42E−06 | 3.35E−06 | >20 μM |
| 372 | 28% | 33% | 19% | >20 μM |
| 373 | 1.52E−06 | 6.20E−06 | 9.21E−06 | >20 μM |
| 374 | 1.60E−06 | 2.55E−06 | 6.50E−06 | >20 μM |
| 375 | 46% | 17% | 35% | >20 μM |
| 376 | 5.46E−06 | 1.84E−05 | 1.79E−05 | >20 μM |
| 377 | 5.14E−06 | 9.75E−06 | 1.51E−05 | >20 μM |
| 378 | 4.30E−07 | 1.05E−06 | 1.45E−06 | >20 μM |
| 379 | 5.68E−07 | 1.35E−06 | 1.63E−06 | >20 μM |
| 380 | 9.37E−07 | 1.60E−06 | 2.74E−06 | >20 μM |
| 381 | 4.90E−07 | 1.90E−06 | 2.33E−06 | >20 μM |
| 382 | 2.53E−07 | 9.31E−07 | 7.48E−07 | >20 μM |
| 383 | 9.84E−07 | 3.62E−06 | 5.11E−06 | >20 μM |
| 384 | 1.23E−7 | 6.65E−8 | 8.46E−8 | n.d. |
| 385 | 7.32E−7 | 3.29E−7 | 3.88E−7 | n.d. |
| 386 | 8.77E−6 | 3.66E−6 | 1.01E−5 | n.d. |
| 387 | 1.17E−6 | 1.66E−6 | 8.32E−6 | >2.00E−5 |
| 388 | 2.05E−7 | 8.01E−7 | 7.57E−7 | >2.00E−5 |
| 389 | 6.69E−6 | 1.49E−5 | 1.69E−5 | >2.00E−5 |
| 390 | 5.00E−6 | 6.25E−6 | 1.29E−5 | >2.00E−5 |
| 391 | 1.55E−8 | 3.67E−8 | 3.79E−8 | >2.00E−5 |
| 392 | 3.38E−7 | 1.19E−6 | 1.52E−6 | >2.00E−5 |
| 393 | 2.44E−7 | 9.75E−7 | 1.04E−6 | >2.00E−5 |
| 394 | 4.05E−7 | 1.62E−6 | 2.29E−6 | >2.00E−5 |
| 395 | 9.84E−7 | 1.06E−6 | 1.02E−6 | 7.07E−6 |
| 396 | 2.24E−6 | 1.15E−6 | 3.13E−6 | >2.00E−5 |
| 397 | 2.97E−7 | 2.09E−7 | 2.49E−7 | >2.00E−5 |
| 398 | 1.04E−7 | 6.34E−8 | 7.21E−8 | >2.00E−5 |
| 399 | 3.33E−7 | 1.92E−7 | 2.96E−7 | >2.00E−5 |
| 400 | 7.87E−8 | 6.88E−8 | 7.66E−8 | >2.00E−5 |
| 401 | 7.63E−8 | 6.52E−8 | 8.23E−8 | >2.00E−5 |
| 402 | 8.28E−8 | 7.52E−8 | 9.81E−8 | >2.00E−5 |
| 403 | 4.42E−7 | 5.48E−7 | 6.32E−7 | >2.00E−5 |
| 404 | 4.68E−6 | 1.45E−6 | 9.30E−6 | >2.00E−5 |
| 405 | 1.11E−6 | 4.48E−7 | 1.31E−6 | >2.00E−5 |
| 406 | 7.27E−7 | 4.00E−7 | 8.55E−7 | >2.00E−5 |
| 407 | 1.25E−6 | 1.06E−6 | 2.14E−6 | >2.00E−5 |
| 408 | 5.33E−7 | 5.60E−7 | 1.02E−6 | >2.00E−5 |
| 409 | 4.92E−8 | 2.80E−8 | 3.16E−8 | >2.00E−5 |
| 410 | 4.23E−6 | 2.79E−6 | >2.00E−5 | >2.00E−5 |
| 411 | 1.45E−5 | >2.00E−5 | >2.00E−5 | >2.00E−5 |
| 412 | n.d. | n.d. | n.d. | n.d. |
| 413 | 2.23E−7 | 2.62E−7 | 2.13E−7 | >2.00E−5 |
| 414 | 1.38E−6 | 1.24E−6 | 1.46E−6 | >2.00E−5 |
| 415 | 1.15E−7 | 8.85E−8 | 9.42E−8 | >2.00E−5 |
| 416 | 8.67E−7 | 6.97E−7 | 1.14E−6 | >2.00E−5 |
| 417 | 4.55E−8 | 2.89E−8 | 3.53E−8 | >2.00E−5 |
| 418 | 8.66E−8 | 3.98E−8 | 4.51E−8 | >2.00E−5 |
| 419 | 6.45E−6 | 3.74E−6 | 1.43E−5 | >2.00E−5 |
| 420 | 3.03E−7 | 3.84E−7 | 5.79E−7 | >2.00E−5 |
| 421 | 1.45E−7 | 2.22E−7 | n.d. | >2.00E−5 |
| 422 | 1.47E−6 | 9.14E−7 | 3.60E−6 | >2.00E−5 |
| 423 | 2.04E−7 | 1.87E−7 | 3.03E−7 | >2.00E−5 |
| 424 | 1.59E−6 | 1.41E−6 | 3.76E−6 | >2.00E−5 |
| 425 | 5.82E−6 | 2.56E−6 | 1.12E−5 | >2.00E−5 |
| 426 | 3.32E−8 | 2.30E−8 | 3.15E−8 | 4.95E−6 |
| 427 | 5.84E−8 | 4.98E−8 | 7.39E−8 | >2.00E−5 |
| 428 | 9.16E−7 | 7.05E−7 | 1.02E−6 | >2.00E−5 |
| 429 | 6.09E−6 | 3.05E−6 | 5.04E−6 | >2.00E−5 |
| 430 | 5.94E−7 | 3.78E−7 | 5.84E−7 | >2.00E−5 |
| 431 | 3.24E−7 | 2.15E−7 | 3.02E−7 | >2.00E−5 |
| 432 | 1.67E−5 | 1.57E−5 | >2.00E−5 | >2.00E−5 |
| 433 | 7.71E−8 | 7.01E−8 | 7.69E−8 | >2.00E−5 |
| 434 | 2.84E−6 | 2.39E−6 | 2.81E−6 | 1.66E−5 |
| 435 | 8.44E−8 | 5.19E−8 | 6.86E−8 | >2.00E−5 |
| 436 | 2.39E−6 | 1.47E−6 | 1.77E−6 | >2.00E−5 |
| 437 | 1.45E−7 | 9.53E−8 | 1.60E−7 | >2.00E−5 |
| 438 | 9.02E−8 | 6.55E−8 | 1.16E−7 | >2.00E−5 |
| 439 | 2.77E−7 | 1.45E−7 | 2.29E−7 | >2.00E−5 |
| 440 | 7.06E−7 | 5.76E−7 | 9.21E−7 | >2.00E−5 |
| 441 | 1.61E−5 | 3.95E−6 | 1.51E−5 | >2.00E−5 |
| 442 | 4.53E−8 | 3.94E−8 | 4.51E−8 | >2.00E−5 |
| 443 | 1.12E−7 | 6.05E−8 | 6.90E−8 | >2.00E−5 |
| 444 | 13.79% | >2.00E−5 | >2.00E−5 | >2.00E−5 |
| 445 | 14.13% | >2.00E−5 | >2.00E−5 | >2.00E−5 |
| 446 | 24.42% | >2.00E−5 | >2.00E−5 | >2.00E−5 |
| 447 | 19.16% | >2.00E−5 | >2.00E−5 | >2.00E−5 |
| 448 | 22.36% | >2.00E−5 | >2.00E−5 | >2.00E−5 |
| 449 | 50.43% | >2.00E−5 | >2.00E−5 | >2.00E−5 |
| 450 | 45.84% | >2.00E−5 | >2.00E−5 | >2.00E−5 |
| 451 | 29.88% | >2.00E−5 | >2.00E−5 | >2.00E−5 |
| 452 | 23.99% | >2.00E−5 | >2.00E−5 | >2.00E−5 |
| 453 | 27.60% | >2.00E−5 | >2.00E−5 | >2.00E−5 |
| 454 | n.d. | n.d. | n.d. | n.d. |
| 455 | 3.97E−7 | 1.04E−6 | 7.69E−7 | >2.00E−5 |
| 456 | 1.62E−7 | 2.92E−7 | 2.87E−7 | >2.00E−5 |
| 457 | 2.95E−7 | 6.82E−7 | 6.38E−7 | >2.00E−5 |
| 458 | 4.80E−8 | 4.00E−8 | 4.55E−8 | >2.00E−5 |

As exemplified in table 1, the compounds of the present invention inhibit the binding of hSOS1 to KRAS, which was measured in the biochemical KRAS-hSOS1 interaction assay (assay 1). The ability to inhibit the KRAS-hSOS1 interaction results in the inhibition of KRAS activation by the compounds, as measured in biochemical assay 3, which quantifies the hSOS1-mediated nucleotide exchange from KRAS-GDP to KRAS loaded with a fluorescent GTP-analog. Furthermore, the compounds of the present invention show the ability to inhibit the nucleotide exchange reaction catalyzed by hSOS1 in the presence of a high concentration of 50 μM GTP, as measured in assay 2. This ability increases the chance that the compounds will be able to inhibit hSOS1 mediated KRAS-activation inside cells, where high GTP concentrations are present. The chemical structure of the compounds of the present invention is similar to known inhibitors of EGFR-kinase. As shown in table 1, most compounds are inactive against EGFR-kinase up to the highest concentration measured in the assay (>20 μM).

The assay data of the large number of compounds in table 1 gives evidence that compounds which have a pharmacological profile as tested according to assays 1 to 3 and as described in the preceding paragraph will be generally useful to inhibit hSOS1 mediated KRAS-activation inside cells, where high GTP concentrations are present and activity against EGFR-kinase up to highest concentrations (>20 μM) will not be measured in the assay.

Therefore an even further aspect of the present invention refers to the use of a compound which inhibits the binding of hSOS1 to H- or N- or K-RAS including their clinically known mutations and which inhibits the nucleotide exchange reaction catalyzed by hSOS1 in the presence of a concentration of 20 μM or lower, but which is substantially inactive against EGFR-kinase at concentrations of 20 μM or lower for the preparation of a medicament for the treatment or prophylaxis of a hyperproliferative disorder.

Particularly this aspect refers to the use of a compound which inhibits the binding of hSOS1 specifically to K-RAS G12C protein and which inhibits the nucleotide exchange reaction catalyzed by hSOS1 in the presence of a concentration of 20 μM or lower, but which is substantially inactive against EGFR-kinase at concentrations of 20 μM or lower for the preparation of a medicament for the treatment or prophylaxis of a hyperproliferative disorder.

Expression and Purification of hK-Ras:hSOS1 and Crystal Structure Determination of the Compound of Example 200 in Complex with hK-Ras:hSOS1

Expression of hK-RasG12C, K-RasG12C-C118S-D126E-T127S-K128R, hSOS1 and hSOS2 in E. coli:

The applied DNA expression constructs encoding the following protein sequences and its corresponding DNA sequences were optimized for expression in E. coli and synthesized by the GeneArt Technology at Life Technologies:
Human K-Ras (P01116-2): hK-Ras_G12C (amino acid 1-169) and K-RasG12C-C118S-D126E-T127S-K128R (amino acid 1-169)
Human SOS1 (Q07889): hSOS1 (amino acid 564-1049)
Human SOS2 (Q07890): hSOS2 (amino acid 564-1043)

These expression constructs additionally encoded att-site sequences at the 5′ and 3′ ends for subcloning into various destination vectors using the Gateway Technology as well as a TEV (Tobacco Etch Virus) protease site for proteolytic cleavage of tag sequences. The applied destination vectors were: pD-ECO1 (an in-house derivate of the pET vector series from Novagen with ampicillin resistance gene) which provides a N-terminal fusion of a GST-tag to the integrated gene of interest. pD-ECO5 (also an in-house derivative of the pET vector series with ampicillin resistance gene) which provides a N-terminal fusion of a His10-tag (SEQ ID NO: 6) to the integrated gene. To generate the final expression vectors the expression construct of hK-RasG12C was cloned into pD-ECO1. hK-RasG12C-C1185-D126E-T1275-K128R and hSOS1 as well as hSOS2 were cloned into pD-ECO5. The resulting expression vectors were termed pD-ECO1_hK-RasG12C, pD-ECO5_hK-RasG12C-C1185-D126E-T1275-K128R, pD-ECO5_hSOS1, pD-ECO5_hSOS2

Sequences:
GST-hK-RasG120 (G12C mutation according to
numbering in P01116-2)
(SEQ ID NO: 7)
MSPILGYWKI KGLVQPTRLL LEYLEEKYEE HLYERDEGDK
WRNKKFELGL EFPNLPYYID GDVKLTQSMA IIRYIADKHN
MLGGCPKERA EISMLEGAVL DIRYGVSRIA YSKDFETLKV
DFLSKLPEML KMFEDRLCHK TYLNGDHVTH PDFMLYDALD
VVLYMDPMCL DAFPKLVCFK KRIEAIPQID KYLKSSKYIA
WPLQGWQATF GGGDHPPKSD PITSLYKKAG SDYDIPTTEN
LYFQGMTEYK LVVVGACGVG KSALTIQLIQ NHFVDEYDPT
IEDSYRKQVV IDGETCLLDI LDTAGQEEYS AMRDQYMRTG
EGFLCVFAIN NTKSFEDIHH YREQIKRVKD SEDVPMVLVG
NKCDLPSRTV DTKQAQDLAR SYGIPFIETS AKTRQGVDDA
FYTLVREIRK HKEK
His10-hK-RasG12C-C118S-D126E-T127S-K128R
(mutations according to numbering
in P01116-2
(SEQ ID NO: 1)
MGHHHHHHHH HHSSGHIEGR HMLETSLYKK AGSDYDIPTT
ENLYFQGMTE YKLVVVGACG VGKSALTIQL IQNHFVDEYD
PTIEDSYRKQ VVIDGETCLL DILDTAGQEE YSAMRDQYMR
TGEGFLCVFA INNTKSFEDI HHYREQIKRV KDSEDVPMVL
VGNKSDLPSR TVESRQAQDL ARSYGIPFIE TSAKTRQGVD
DAFYTLVREI RKHKEK
His10-hSOS1
(SEQ ID NO: 3)
MGHHHHHHHH HHSSGHIEGR HMLETSLYKK AGSDYDIPTT
ENLYFQGEEQ MRLPSADVYR FAEPDSEENI IFEENMQPKA
GIPIIKAGTV IKLIERLTYH MYADPNFVRT FLTTYRSFCK
PQELLSLIIE RFEIPEPEPT EADRIAIENG DQPLSAELKR
FRKEYIQPVQ LRVLNVCRHW VEHHFYDFER DAYLLQRMEE
FIGTVRGKAM KKWVESITKI IQRKKIARDN GPGHNITFQS
SPPTVEWHIS RPGHIETFDL LTLHPIEIAR QLTLLESDLY
RAVQPSELVG SVWTKEDKEI NSPNLLKMIR HTTNLTLWFE
KCIVETENLE ERVAVVSRII EILQVFQELN NFNGVLEVVS
AMNSSPVYRL DHTFEQIPSR QKKILEEAHE LSEDHYKKYL
AKLRSINPPC VPFFGIYLTN ILKTEEGNPE VLKRHGKELI
NFSKRRKVAE ITGEIQQYQN QPYCLRVESD IKRFFENLNP
MGNSMEKEFT DYLFNKSLEI EPRNPKPLPR FPKKYSYPLK
SPGVRPSNPR PGT
His10-hSOS2
(SEQ ID NO: 8)
MGHHHHHHHH HHSSGHIEGR HMLETSLYKK AGSDYDIPTT
ENLYFQGPLR LPSPEVYRFV VKDSEENIVF EDNLQSRSGI
PIIKGGTVVK LIERLTYHMY ADPNFVRTFL TTYRSFCKPQ
ELLSLLIERF EIPEPEPTDA DKLAIEKGEQ PISADLKRFR
KEYVQPVQLR ILNVFRHWVE HHFYDFERDL ELLERLESFI
SSVRGKAMKK WVESIAKIIR RKKQAQANGV SHNITFESPP
PPIEWHISKP GQFETFDLMT LHPIEIARQL TLLESDLYRK
VQPSELVGSV WTKEDKEINS PNLLKMIRHT TNLTLWFEKC
IVEAENFEER VAVLSRIIEI LQVFQDLNNF NGVLEIVSAV
NSVSVYRLDH TFEALQERKR KILDEAVELS QDHFKKYLVK
LKSINPPCVP FFGIYLTNIL KTEEGNNDFL KKKGKDLINF
SKRRKVAEIT GEIQQYQNQP YCLRIEPDMR RFFENLNPMG
SASEKEFTDY LFNKSLEIEP RNCKQPPRFP RKSTFSLKSP
GIRPNTG

E. coli Expression:

The expression vectors were transformed into E. coli strain BL21 (DE3). Cultivation of the transformed strains for expression was done in 10 L and 1 L fermenter.

The cultures were grown in Terrific Broth media (MP Biomedicals, Kat. #113045032) with 200 μg/mL ampicillin at a temperature of 37° C. to a density of 0.6 (00600), shifted to a temperature of 27° C. (for hK-Ras expression vectors) or 17° C. (for hSOS expression vectors), induced for expression with 100 mM IPTG and further cultivated for 24 hours.

Purification

After cultivation the transformed E. coli were harvested by centrifugation and the resulting pellet was suspended in a lysis buffer (see below) and lysed by passing three-times through a high pressure device (Microfluidics). The lysate was centrifuged (49000 g, 45 min, 4° C.) and the supernatant used for further purification.

An Äkta chromatography system was used for all further chromatography steps.

Purification of GST-hK-RasG12C for Biochemical Assays

E. coli culture (transformed with pD-ECO1_hK-RasG120) from a 10 L fermenter was lysed in lysis buffer (50 mM Tris HCl 7.5, 500 mM NaCl, 1 mM DTT, 0.5% CHAPS, Complete Protease Inhibitor Cocktail, (Roche)). As a first chromatography step the centrifuged lysate was incubated with 50 mL Glutathione Agarose 4B (Macherey-Nagel; 745500.100) in a spinner flask (16 h, 10° C.). The Glutathione Agarose 4B loaded with protein was transferred to a chromatography column connected to an Äkta chromatography system. The column was washed with wash buffer (50 mM Tris HCl 7.5, 500 mM NaCl, 1 mM DTT) and the bound protein eluted with elution buffer (50 mM Tris HCl 7.5, 500 mM NaCl, 1 mM DTT, 15 mM Glutathione). The main fractions of the elution peak (monitored by OD280) were pooled.

For further purification by size-exclusion chromatography the above eluate volume was applied to a column Superdex 200 HR prep grade (GE Healthcare) and the resulting peak fractions of the eluted fusion protein were collected. The final yield of hK-RasG12C was about 50 mg purified fusion protein per L culture and the final product concentration was about 1 mg/mL. Native mass spectrometry analyses of the final purified K-RasG12C demonstrated its homogeneous load with GDP.

Purification of His10-hSOS1 and His10-hSOS2 for Biochemical Assays

E. coli transformed with pD-ECO5_hSOS1 or pD-ECO5_hSOS2 were cultured and induced in a fermenter, harvested and lysed in lysis buffer (25 mM Tris HCl 7.5, 500 mM NaCl, 20 mM Imidazol, Complete EDTA-free (Roche)). For immobilized metal ion affinity chromatography (IMAC) the centrifuged lysate was incubated with 30 mL Ni-NTA (Macherey-Nagel; #745400.100) in a spinner flask (16 h, 4° C.) and subsequently transferred to a chromatography column connected to an Äkta chromatography system. The column was rinsed with wash buffer (25 mM Tris HCl 7.5, 500 mM NaCl, 20 mM Imidazol) and the bound protein eluted with a linear gradient (0-100%) of elution buffer (25 mM Tris HCl 7.5, 500 mM NaCl, 300 mM Imidazol). The main fractions of the elution peak (monitored by OD280) containing homogenous His10-hSOS were pooled. The final yield of His10-hSOS1 was about 110 mg purified protein per L culture and the final product concentration was about 2 mg/mL. For His10-hSOS2 the final yield was 190 mg per L culture and the product concentration 6 mg/mL.

Generation complex of hK-RasG12C-C1185-D126E-T1275-K128R/hSOS1 for crystallography Purification of tag-free hK-RasG12C-C1185-D126E-T1275-K128R A process consisting of 4 chromatography steps with an Äkta system was applied to produce tag-free hK-RasG12C-C1185-D126E-T1275-K128R.

An E. coli BL21 (DE3) strain transformed with pD-ECO5_hK-RasG12C-C118S-D126E-T127S-K128R was cultured and induced in a fermenter and the resulting culture was harvested and lysed in lysis buffer (50 mM Tris HCl 7.5, 300 mM NaCl, 10 mM Imidazol, 0.5% CHAPS, Complete-Protease Inhibitor Cocktail-EDTA-free (Roche)). The centrifuged lysate was applied to two linked 5 mL columns of Protino Ni-NTA (Macherey-Nagel; #745415.5) connected to an Äkta chromatography system. Subsequently the column was rinsed with wash buffer (50 mM Tris HCl 7.5, 300 mM NaCl, 10 mM Imidazol) and the bound protein eluted with a linear gradient (0-100%) of elution buffer (50 mM Tris HCl 7.5, 300 mM NaCl, 300 mM Imidazol). The main fractions of the elution peak (monitored by OD280) containing His10-hK-RasG12C-C1185-D126E-T127S-K128R were pooled and applied to a HiPrep Desalting column (GE; #17-5087 01) to change the solution to the cleavage buffer (50 mM Tris HCl 7.5, 150 mM NaCl, 1 mM DTT). This protein solution was than treated with purified His-TEV protease (ratio hK-Ras:TEV, w/w, 30:1) for 16 h at 4° C. and afterwards passed over a Ni-NTA column to remove non-cleaved hKRas protein, cleaved tag and His-TEV. The processed hK-Ras protein in the pooled flow-through fractions was concentrated using the device Amicon Ultra 15 Ultracel-3 (Centrifugal Filter 3000 NMWL, Merck-Millipore #UFC900324) and applied to size-exclusion chromatography column with Superdex 200 HR prep grade (GE Healthcare) in SEC buffer (20 mM HEPES 8.0, 150 mM NaCl, 1 mM DTT). The final yield of processed hK-RasG12C-C1185-D126E-T1275-K128R was about 70 mg protein per 10 L culture and the final product concentration was about 4 mg/mL. This final product was homogeneously loaded with GDP as demonstrated by native mass spectrometry analyses.

Purification of Tag-Free hSOS1

A process consisting of 4 chromatography steps with an Äkta system was used to produce tag-free hSOS1.

His10-hK-RasG12C was expressed in E. coli (transformed with pD-ECO5_hSOS1) as described for biochemical assays. For IMAC the centrifuged lysate was directly applied to a 30 mL column with Ni-NTA (Macherey-Nagel) in an Äkta system, rinsed with wash buffer (25 mM Tris HCl 7.5, 500 mM NaCl, 20 mM Imidazol) and the bound protein was eluted with a linear gradient (0-100%) of elution buffer (25 mM Tris HCl 7.5, 500 mM NaCl, 300 mM Imidazol). The main fractions of the elution peak (monitored by OD280) were passed over a HiPrep Desalting column (GE; #17-5087-01) to change to the cleavage buffer (25 mM Tris HCl 7.5, 150 mM NaCl, 1 mM DTT). The adjusted protein solution was treated with purified His-TEV protease (ratio hSOS1:TEV, w/w, 30:1) for 16 h at 4° C. and afterwards passed over a Ni-NTA column to remove non-cleaved hSOS1 protein, cleaved tag and His-TEV. The pooled flow through fractions with the processed hSOS1 were concentrated using a Amicon Ultra 15 Ultracel-10 device (Centrifugal Filter 10000 NMWL; Merck-Millipore #UFC901024) and applied to size-exclusion chromatography column with Superdex 200 HR prep grade (GE Healthcare) in SEC buffer (25 mM Tris HCl 7.5, 100 mM NaCl). The final yield of processed hSOS1 was about 275 mg protein per 1 L culture and the final product concentration was about 3.5 mg/mL.

Formation and Purification of a Complex of hK-Ras/hSOS1 for Crystallization

Tag-free hK-RasG12C-C1185-D126E-T1275-K128R was concentrated to 40 mg/mL (2 mL) and tag-free hSOS1 to 44 mg/mL (1.5 mL) using Amicon concentration devices as described before. As efficient K-Ras/SOS1 complex formation requires removal of GDP the concentrated hK-RasG12C-C1185-D126E-T1275-K128R solution was adjusted to 200 mM (NH₄)₂SO₄ and 0.1 mM ZnCl₂ and incubated with 75 units of alkaline phosphatase immobilized to agarose (SIGMA; #P0762) and incubated for 1 h at 4° C. in an overhead rotating tube. Afterwards the concentrated tag-free hSOS1 sample was added on a basis of a molar ratio of hK-Ras:hSOS1 of 3:1 and incubated for 16 h at 4° C. in an overhead rotating tube. The suspension was cleared by centrifugation (1.000 g, 10 min, 4° C.) and the supernatant applied to HiPrep desalting column to adjust to the SEC buffer (5 mM Tris HCl 7.5, 100 mM NaCl). The adjusted protein solution was concentrated using an Amicon Ultra 15 Ultracel-10 device (Centrifugal Filter 10000 NMWL; Merck-Millipore #UFC901024) and loaded on a preparative Superdex S200 column in SEC buffer (5 mM Tris HCl 7.5, 100 mM NaCl) thereby enabling an efficient separation of the hK-Ras/hSOS1 complex from the abundant hK-Ras. The fractions with the complex were concentrated so that finally 37 mg of hK-RasG12C-C1185-D126E-T1275-K128R/hSOS1 complex at a concentration of 14.4 mg/mL was achieved. Complex formation was further demonstrated by analytical SEC (S200 5/150 GL; GE; #28-9065-61) as with this method the retention times of the elution peaks for the complexed proteins and individual hK-Ras and hSOS1 component could be clearly distinguished.

Crystallization of K-Ras:hSOS1 Complex

For crystallization, frozen aliquots of the hK-RasG12C-C1185-D126E-T1275-K128R/hSOS1 complex (in the following called “hK-Ras:hSOS1”, total protein concentration 14.4 mg/ml, sequences see FIGS. X2 and X4) were thawed and crystals were grown at 4° C. using the hanging drop method. Drops were made from 1 μl protein solution (in 5 mM Tris pH 7.5; 100 mM NaCl) and 1 μl reservoir solution (2.9-3.4 M sodium formate, 100 mM MES pH 6.5). Small crystals grew within several days. Crystals large enough to obtain high resolution diffraction data appeared only after about 30 days.

Complex Formation of K-Ras:SOS1 and the Compound of Example 200 in the Crystal

For complex formation, a crystal was transferred into a new drop of 1.5 μl storage buffer solution (3.5 M sodium formate, 100 mM MES pH 6.5) and stored over a reservoir solution consisting of 3.5 M sodium formate, 100 mM MES pH 6.5, 5% DMSO. A stock solution of the compound of Example 200 (200 mM in DMSO) was 10-fold diluted with reservoir solution. Over the course of 2 hours, 1.5 μl of this diluted stock solution were added in three steps of 0.5 μl to the drop containing the hK-Ras:hSOS1 crystal, resulting in a final concentration of 10 mM of the compound of Example 200 in the soaking drop. The crystal was soaked in this drop for 1.5 days at 4° C.

Data Collection and Processing

The soaked crystal was briefly immerged in cryo buffer (0.1 M MES pH 6.5, 3.5 M sodium formate, 20% glycerol and 2 mM of the compound of Example 200) and shock frozen in liquid nitrogen. A diffraction data set was collected at beamline 14.1 at Helmholtz-Zentrum Berlin at 100 K using a wavelength of 0.91841 Å and a PILATUS detector. The diffraction images were processed using the program XDS. The crystal diffracted to a resolution of 2.4 Å and belonged to space group 1422 with unit cell dimensions of a=b=149.3 Å and c=202.4 Å, with one hK-Ras:hSOS1 complex per asymmetric unit.

Structure Determination and Refinement

The crystal form described here was first obtained and solved for a hK-Ras:hSOS1 crystal in the absence of any inhibitor (apo structure), using the Molecular Replacement method with the program PHASER from the CCP4 program suite and the published structure of the complex of human H-Ras and hSOS1 (PDB entry 1BKD, ref.: Boriack-Sjodin P A, Margarit S M, Bar-Sagi D, Kuriyan J. The structural basis of the activation of Ras by Sos. Nature. 1998 Jul. 23; 394(6691):337-43) as search model. The data set for hK-Ras:hSOS1:compound of Example 200 was then solved by rigid body refinement using the hK-Ras:hSOS1 apo structure as starting model and the program REFMAC as part of the CCP4 program suite. A 3D model for the compound Example 200 was generated using the program Discovery Studio (company Biovia) and parameter files for crystallographic refinement and model building were generated using software PRODRG. The compound of Example 200 was manually built into the electron density maps using the program COOT, followed by several cycles of refinement (using program REFMAC) and rebuilding in COOT. The final co-complex structure features an R(work) factor of 19.8% and an R(free) factor of 24.3%. The statistics of the data collection and refinement are summarized in Table 2.

TABLE 2
Data collection and refinement statistics for
the complex of hK-Ras:hSOS1 and Example 200

| | hK-Ras:hSOS1: Example 200 | |
|---|---|---|
| Data Collection: | | |
| Source | BL 14.1 (Helmholtz- | |
| | Zentrum Berlin) | |
| Wavelength [Å] | 0.9184 | |
| Space group (no.) | I4222 | (97) |
| Unit cell parameters, | 149.3 149.3 202.4 | |
| a, b, c [Å] | | |
| Resolution limit [Å] | 2.40-48.32 | (2.40-2.54) |
| No. of reflections | 469725 | |
| No. of uniques | 44010 | |
| Multiplicity | 10.67 | |
| I/sigl | 9.58 | (1.65) |
| R_meas [%] | 20.7 | (115.5) |
| Completeness [%] | 97.8 | (91.3) |
| B(Wilson) [Å²] | 45.2 | |
| Mosaicity [deg] | 0.071 | |
| Refinement | | |
| Resolution limit [Å] | 2.40-48.32 | (2.40-2.46) |
| Completeness [%] | 97.8 | (81.1) |
| No. of reflections | 44009 | (41908/2101) |
| (work/test) | | |
| R (work)/R(free) [%] | 19.8/24.3 | (37.4/42.8) |

| | |
|---|---|
| Mean B value [Å²] | 48.7 |
| RMSD bond lengths [Å] | 0.017 |
| RMSD bond angles [deg] | 1.85 |

Values in brackets refer to the highest resolution shell.

Absolute Configuration of the Compound of Example 200 in Human hK-Ras:hSOS1

The complex of hK-Ras:hSOS1 and the compound of Example 200 crystallizes with one hK-Ras:hSOS1 complex in the asymmetric unit. The stereo chemistry of the compound of Example 200 is unambiguously defined by the knowledge of the stereo chemistry of the protein complex of hK-Ras:hSOS1. The compound of Example 200 unambiguously features the R configuration on carbon atom C17 (FIG. X5).

FIG. X1: Sequence of His10-hK-Ras with mutations G12C-C1185-D126E-T1275-K128R (mutations according to numbering in P01116-2) before cleavage by TEV protease.

(SEQ ID NO: 1)
MGHHHHHHHH HHSSGHIEGR HMLETSLYKK AGSDYDIPTT
ENLYFQGMTE YKLVVVGACG VGKSALTIQL IQNHFVDEYD
PTIEDSYRKQ VVIDGETCLL DILDTAGQEE YSAMRDQYMR
TGEGFLCVFA INNTKSFEDI HHYREQIKRV KDSEDVPMVL
VGNKSDLPSR TVESRQAQDL ARSYGIPFIE TSAKTRQGVD
DAFYTLVREI RKHKEK

FIG. X2: Sequence of His10-hK-Ras with mutations G12C-C1185-D126E-T1275-K128R after cleavage by TEV protease.

(SEQ ID NO: 2)
YKLVVVGACG VGKSALTIQL IQNHFVDEYD PTIEDSYRKQ
VVIDGETCLL DILDTAGQEE YSAMRDQYMR TGEGFLCVFA
INNTKSFEDI HHYREQIKRV KDSEDVPMVL VGNKSDLPSR
TVESRQAQDL ARSYGIPFIE TSAKTRQGVD DAFYTLVREI
RKHKEK

FIG. X3: Sequence of hSOS1 with N-terminal His tag (His10-hSOS1) before cleavage by TEV protease.

(SEQ ID NO: 3)
MGHHHHHHHH HHSSGHIEGR HMLETSLYKK AGSDYDIPTT
ENLYFQGEEQ MRLPSADVYR FAEPDSEENI IFEENMQPKA
GIPIIKAGTV IKLIERLTYH MYADPNFVRT FLTTYRSFCK
PQELLSLIIE RFEIPEPEPT EADRIAIENG DQPLSAELKR
FRKEYIQPVQ LRVLNVCRHW VEHHFYDFER DAYLLQRMEE
FIGTVRGKAM KKWVESITKI IQRKKIARDN GPGHNITFQS
SPPTVEWHIS RPGHIETFDL LTLHPIEIAR QLTLLESDLY
RAVQPSELVG SVWTKEDKEI NSPNLLKMIR HTTNLTLWFE
KCIVETENLE ERVAVVSRII EILQVFQELN NFNGVLEVVS
AMNSSPVYRL DHTFEQIPSR QKKILEEAHE LSEDHYKKYL
AKLRSINPPC VPFFGIYLTN ILKTEEGNPE VLKRHGKELI
NFSKRRKVAE ITGEIQQYQN QPYCLRVESD IKRFFENLNP
MGNSMEKEFT DYLFNKSLEI EPRNPKPLPR FPKKYSYPLK
SPGVRPSNPR PGT

FIG. X4: Sequence of hSOS1 after cleavage by TEV protease.

(SEQ ID NO: 4)
GEEQ
MRLPSADVYR FAEPDSEENI IFEENMQPKA GIPIIKAGTV
IKLIERLTYH MYADPNFVRT FLTTYRSFCK PQELLSLIIE
RFEIPEPEPT EADRIAIENG DQPLSAELKR FRKEYIQPVQ
LRVLNVCRHW VEHHFYDFER DAYLLQRMEE FIGTVRGKAM
KKWVESITKI IQRKKIARDN GPGHNITFQS SPPTVEWHIS
RPGHIETFDL LTLHPIEIAR QLTLLESDLY RAVQPSELVG
SVWTKEDKEI NSPNLLKMIR HTTNLTLWFE KCIVETENLE
ERVAVVSRII EILQVFQELN NFNGVLEVVS AMNSSPVYRL
DHTFEQIPSR QKKILEEAHE LSEDHYKKYL AKLRSINPPC
VPFFGIYLTN ILKTEEGNPE VLKRHGKELI NFSKRRKVAE
ITGEIQQYQN QPYCLRVESD IKRFFENLNP MGNSMEKEFT
DYLFNKSLEI EPRNPKPLPR FPKKYSYPLK SPGVRPSNPR
PGT

FIG. X5: Structure of the compound of Example 200 in complex with human hK-Ras:hSOS1. Carbon atom C17 unambiguously features R configuration.

### LITERATURE

1.) Boriack-Sjodin PA, Margarit SM, Bar-Sagi D, Kuriyan J. The structural basis of the activation of Ras by Sos. Nature. 1998 Jul. 23; 394(6691):337-43.

### References for the Crystallographic Software Tools

CCP4: M. D. Winn et al. Acta. Cryst. D67, 235-242 (2011) “Overview of the CCP4 suite and current developments”
Phaser: J. Appl. Cryst. (2007). 40, 658-674. Phaser crystallographic software. McCoy, A. J., Grosse-Kunstleve, R. W., Adams, P. D., Winn, M. D., Storoni, L. C., & Read, R. J.
Refmac: “Refinement of Macromolecular Structures by the Maximum-Likelihood method” G. N. Murshudov, A. A. Vagin and E. J. Dodson, (1997) in Acta Cryst. D53, 240-255.
ProDrg: A. W. Schüttelkopf and D. M. F. van Aalten (2004). “PRODRG: a tool for high-throughput crystallography of protein-ligand complexes”, Acta Crystallogr D60, 1355-1363.
COOT: Paul Emsley, Bernhard Lohkamp, William G. Scott, Kevin Cowtan, ”
Features and Development of Coot”, (2010) Acta Cryst. D66:486-501
XDS: Kabsch, W. XDS. Acta Cryst. D66, 125-132 (2010).

What is claimed is:

1. A compound of general formula (I): 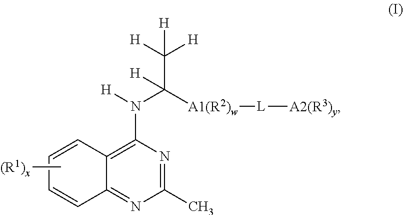
in which R¹ stands for:
a substituent independently selected from: a hydrogen atom, a halogen atom, a hydroxy, cyano, nitro, C₁-C₆-alkylsulfanyl, an amino group —NR^{a}R^{b}, wherein R^{a} and R^{b} are selected independently from a hydrogen atom or a C₁-C₆-alkyl,
a substituent selected from: a C₁-C₆-alkyl, C₁-C₆-alkoxy-, C₂-C₆-alkenyl, C₂-C₆-alkynyl, C₃-C₈-cycloalkyl, C₄-C₈-cycloalkenyl, 4- to 7-membered heterocycloalkyl, 5- to 10-membered heterocycloalkenyl, heterospirocycloalkyl, fused heterocycloalkyl, bridged heterocycloalkyl, phenyl, heteroaryl, C₁-C₆-haloalkyl,
—C(═O)OH, —C(═O)OR^{c}, and wherein R^{c} stands for C₁-C₆-alkyl, C₃-C₆-alkenyl, C₃-C₆-alkynyl, C₃-C₈-cycloalkyl or C₄-C₈-cycloalkenyl,
—N═S(═O)(R^{d})R^{e}, and wherein R^{d} and R^{e} are independently selected from hydrogen, C₁-C₆-alkyl, C₂-C₆-alkenyl, C₂-C₆-alkynyl, C₃-C₈-cycloalkyl or C₄-C₈-cycloalkenyl,
—NH—C(O)—C₁-C₆-alkyl, —NH—C(O)—NR^{a}R^{b}, wherein R^{a} and R^{b} are selected independently from a hydrogen atom or a C₁-C₆-alkyl, —NH—(CH₂)k-NH—C(O)—C₁-C₆-alkyl, wherein k is 1 or 2, *—NH—(CH₂)ₗ—R^{f}, wherein l is 0, 1 or 2 and R^{f} stands for a 4- to 7-membered heterocycloalkyl, heteroaryl, C₁-C₆-alkylsulfonyl,
whereby in all foregoing definitions the C₁-C₆-alkyl-, C₁-C₆-alkoxy-, the 4- to 7-membered heterocycloalkyl and the heteroaryl can be optionally substituted, one, two or three times, identically or differently, with:
a halogen atom, or a group selected from hydroxy, oxo (═O), a cyano, nitro, C₁-C₆-alkyl, C₂-C₆-alkenyl, C₂-C₆-alkynyl, C₃-C₈-cycloalkyl, 4- to 7-membered heterocycloalkyl, C₁-C₆-alkoxy, C₁-C₆-haloalkyl, C₁-C₆-haloalkoxy-, C₁-C₆-alkylsulfonyl, phenyl, benzyl-, heteroaryl, —(CH₂)-heteroaryl, C₃-C₈-cycloalkoxy-, phenyloxy-, heteroaryloxy-, —NH—C(O)—C₁-C₆-alkyl or an amino group —NR^{a}R^{b}, wherein R^{a} and R^{b} are selected independently from a hydrogen atom or a C₁-C₆-alkyl, or
a substituent —O—(CH₂)_{z}-phenyl, —O—(CH₂)_{z}—C₄-C₇-heterocycloalkyl, —O—(CH₂)_{z}-heteroaryl, whereby
z is 0, 1 or 2, and the phenyl, heterocycloalkyl and heteroaryl can optionally be substituted with a group selected from hydroxy, heterocycloalkyl or heterocycloalkenyl, which both can be substituted with a methyl- and/or oxo-group,
or a substituent selected from the group of
NHS(O)₂—CH₃, 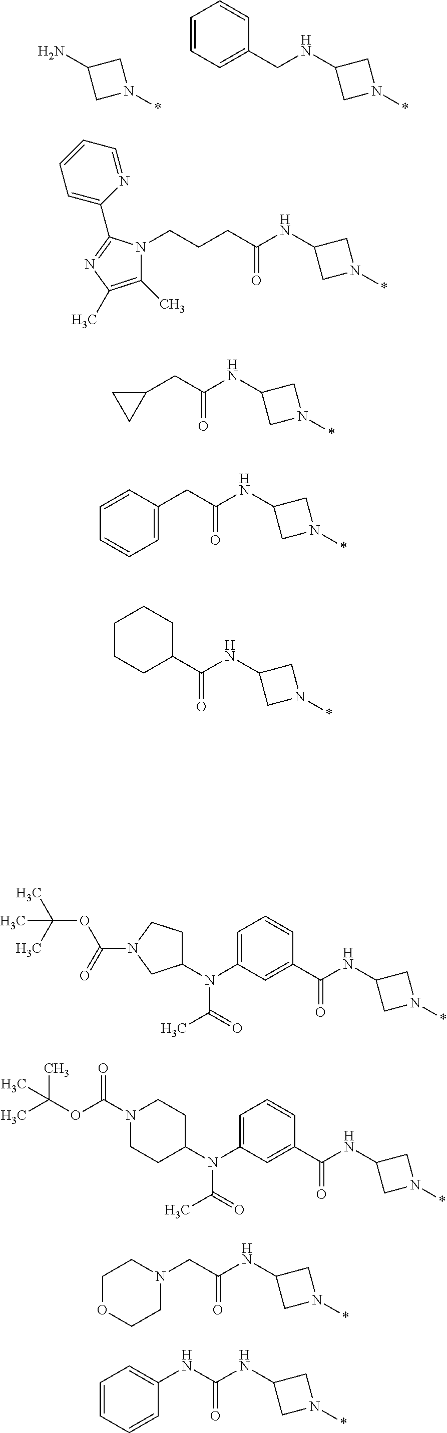 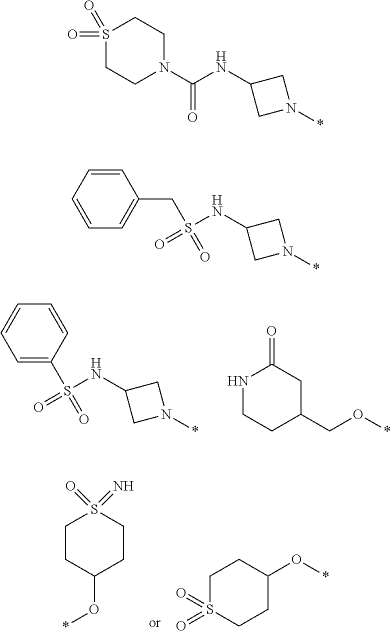 [SMILES: *N1CC(NC(=O)N2CCS(=O)(=O)CC2)C1 ; *N1CC(NS(=O)(=O)Cc2ccccc2)C1 ; *N1CC(NS(=O)(=O)c2ccccc2)C1 ; *OCC1CCNC(=O)C1 ; *OC1CCS(=N)(=O)CC1 ; *OC1CCS(=O)(=O)CC1]
and wherein x is 1, 2 or 3,
A1 is aryl,
R² stands for:
a hydrogen atom, a hydroxy group, a halogen atom, a cyano group, a substituent selected from a C₁-C₆-alkyl, C₁-C₆-alkoxy-, C₂-C₆-alkenyl, 4- to 7-membered heterocycloalkyl, —O—CH₂-4- to 7-membered heterocycloalkyl, 5- to 10-membered heterocycloalkenyl, heterospirocycloalkyl, fused heterocycloalkyl, bridged heterocycloalkyl, phenyl, heteroaryl, C₁-C₆-haloalkyl, C₁-C₆-alkylsulfonyl, wherein where R² stands for a halogen atom, the halogen atom is not Cl,
—NR^{a}R^{b}, wherein R^{a} and R^{b} are selected independently from a hydrogen atom or a C₁-C₆-alkyl, wherein R^{a} and R^{b} are both not hydrogen,
—C(O)—NR^{a}R^{b}, wherein R^{a} and R^{b} are selected independently from a hydrogen atom or a C₁-C₆-alkyl, —C(O)—O—R⁹, wherein R⁹ is a hydrogen atom or a C₁-C₆-alkyl, —O—R^{h}, wherein R^{h} is a C₁-C₆-alkyl or —CH₂—NR^{a}R^{b}, wherein R^{a} and R^{b} are selected independently from a hydrogen atom or a C₁-C₆-alkyl, or
—S(O)₂CH₃,
and w is 1 or 2,
and wherein
A2(R³)_{y} stands either for a hydrogen atom or
A2 has the same meanings as the substituent A1 and
R³ stands for
a hydrogen atom, a halogen atom, a hydroxy, cyano, nitro group, a substituent selected from a C₁-C₆-alkyl, C₂-C₆-alkenyl, C₂-C₆-alkynyl, C₃-C₈-cycloalkyl, C₄-C₈ cycloalkenyl, C₇-C₈-cycloalkynyl, 4- to 7-membered heterocycloalkyl, 5- to 10-membered heterocycloalkenyl, phenyl, heteroaryl or C₁-C₆-haloalkyl,
which substituent is optionally substituted, one, two or three times, identically or differently, with a substituent selected from:
a halogen atom, or a group selected from hydroxy, cyano, C₁-C₆-alkyl, C₁-C₆-alkoxy, C₁-C₆-haloalkyl, phenyl, —C(O)NRⁱR^{j}, wherein
Rⁱ and R^{j} are selected independently from a hydrogen atom or a C₁-C₆-alkyl, heteroaryl,
or with amino —NR^{k}R^{l}, wherein R^{k} and R^{l} are selected independently from
a hydrogen atom, a substituent selected from a C₁-C₆-alkyl, C₂-C₆-alkenyl, C₂-C₆-alkynyl, C₃-C₈-cycloalkyl, C₁-C₆-alkylsulfonyl, phenyl, heteroaryl, 4- to 7-membered heterocycloalkyl, which are optionally substituted
one, two or three times, identically or differently, with a substituent selected from C₁-C₆-haloalkyl, hydroxyl, oxo (═O), phenyl, cyano, C₁-C₆-alkoxy, heteroaryl, wherein
the heteroaryl can optionally be substituted with a methyl group, or
—CH₂—C(O)—R^{m}, wherein
R^{m} is a bicyclic heteroaryl, which can be partially hydrogenated, a C₁-C₆-alkoxy or a group —NRⁿR^{o}, in which
Rⁿ and R^{o} are selected independently from hydrogen, C₁-C₆-alkyl, phenyl, wherein the C₁-C₆-alkyl can optionally be substituted with a C₁-C₆-alkoxy or a phenyl, or
RⁿR^{o} stands for a 4- to 7-membered azacycloalkyl, bound via the nitrogen atom to the rest of the molecule and which optionally contains one more heteroatom selected from nitrogen and oxygen;
—C(═O)R^{p}, wherein R^{p} is selected from
the group of a C₁-C₆-alkoxy, a C₁-C₆-alkyl, which is optionally substituted, one, two or three times, identically or differently, with a substituent selected from hydroxyl or C₁-C₆-alkoxy, a mono- or bicyclic heteroaryl, a 4- to 7-membered heterocycloalkyl or R^{p} is a group —CH₂—NR^{q}R^{r}; wherein R^{q} and R^{r} are selected independently from hydrogen, phenyl or a C₁-C₆-alkyl, which may optionally be substituted up to threefold with fluorine,
or —NR^{s}R^{t} is a 4- to 7-membered azacycloalkyl, bound via the nitrogen atom to the rest of the molecule, or a 6- to 10-membered azaspirocycloalkyl, bound via the nitrogen atom to the rest of the molecule and which both may contain up to 2 further heteroatoms selected from nitrogen and oxygen and which both are optionally substituted one, two or three times, identically or differently, with a substituent selected from: hydroxy, oxo (═O), C₁-C₆-alkyl, C₁-C₆-hydroxyalkyl, —C(═O)OR^{u}, wherein R^{u} is a C₁-C₆-alkyl, halogen, —N(C₁-C₆-alkyl)₂, —CH₂—N(C₁-C₆-alkyl)₂, —C(O)NR^{a}R^{b}, wherein R^{a} and R^{b} are selected independently from a hydrogen atom or a C₁-C₆-alkyl,
or a substituent selected from: —C(═O)R^{v}, —C(═O)NH₂, —C(═O)N(H)R^{v}, —C(═O)N(R^{v})R^{w} or —C(═O)OR^{v}, wherein
R^{v} and R^{w} represent, independently from each other, a group selected from hydrogen, C₁-C₄-alkyl, C₁-C₄-haloalkyl, phenyl, or a group —(CH₂)₂—NRₓR_{y}, wherein Rₓ and R_{y} independently from each other stand for hydrogen, a C₁-C₄-alkyl or a group —(CH₂)₂N(CH₃)₂;
or a substituent selected from: —NH₂, —NHR^{z}, —N(R^{z})R^{za}, —N(H)C(═O)R^{z}, —N(H)C(═O)OR^{z}, —N(H)S(═O)₂R^{z}, 4- to 7-membered heterocycloalkyl, heteroaryl, heterospirocycloalkyl, fused heterocycloalkyl- or bridged heterocycloalkyl, wherein
R^{z} and R^{za} represent, independently from each other, a group selected from C₁-C₄-alkyl, C₁-C₄-haloalkyl and phenyl,
or a substituent selected from: —C₁-C₆-alkoxy-, C₁-C₆-haloalkoxy-, —O—(CH₂)ₛ—C₃-C₈-cycloalkyl, —O—(CH₂)ₛ-phenyl, —O—(CH₂)ₛ-heterocycloalkyl or —O—(CH₂)ₛ-heteroaryl, wherein s is 0, 1, 2 or 3,
or a substituent selected from: —S(═O)₂R^{z}, —S(═O)₂NH₂, —S(═O)₂NHR^{z} or —S(═O)₂N(R^{z})R^{za}, wherein R^{z} and R^{za} represent, independently from each other, a group selected from C₁-C₄-alkyl, C₁-C₄-haloalkyl or phenyl,
wherein y is 1, 2 or 3, and
L stands either for a bond or for —O—(CH₂)ₖ, wherein k is 0, 1, 2 or 3, or a group —CH═CH—(CH₂)ₙ, wherein n is 0, 1 or 2,
or a tautomer, an N-oxide, a hydrate, a solvate, or a salt thereof, or a mixture of same.

2. The compound according to claim 1, in which:
in which R¹ stands for:
a substituent independently selected from: a hydrogen atom, a halogen atom, a hydroxy, cyano, nitro, C₁-C₆-alkylsulfanyl, an amino group —NR^{a}R^{b},
wherein R^{a} and R^{b} are selected independently from a hydrogen atom or a C₁-C₆-alkyl,
a substituent selected from: a C₁-C₆-alkyl, C₁-C₆-alkoxy-, C₃-C₈-cycloalkyl, 4- to 7-membered heterocycloalkyl, heteroaryl,
—C(═O)OH, —C(═O)OR^{c}, and wherein R^{c} stands for C₁-C₆-alkyl or C₄-C₈-cycloalkyl,
—N═S(═O)(R^{d})R^{e}, and wherein R^{d} and R^{e} are independently from C₁-C₆-alkyl,
—NH—C(O)—C₁-C₆-alkyl, —NH—C(O)—NR^{a}R^{b}, wherein R^{a} and R^{b} are selected independently from a hydrogen atom or a C₁-C₆-alkyl, —NH—(CH₂)ₖ—NH—C(O)—C₁-C₆-alkyl, wherein k is 1 or 2, *—NH—(CH₂)ₗ—R^{f}, wherein
l is 0, 1 or 2 and R^{f} stands for a 4- to 7-membered heterocycloalkyl or C₁-C₆-alkylsulfonyl,
whereby in all foregoing definitions the C₁-C₆-alkyl-, C₁-C₆-alkoxy-, the 4- to 7-membered heterocycloalkyl and the heteroaryl can be optionally substituted, one or two or three times, identically or differently, with:
a group selected from hydroxy, oxo (═O), C₁-C₆-alkyl, C₃-C₈-cycloalkyl, 4- to 7-membered heterocycloalkyl, C₁-C₆-alkoxy, C₁-C₆-alkylsulfonyl, benzyl, —(CH₂)-heteroaryl- or an amino group —NR^{a}R^{b}, wherein R^{a} and R^{b} are selected independently from C₁-C₆-alkyl, or
a substituent —O—(CH₂)_{z}-phenyl, whereby z is 0, 1 or 2,
R² stands for:
a hydrogen atom, a hydroxy group, a halogen atom, a cyano group, a substituent selected from: a C₁-C₆-alkyl, C₁-C₆-alkoxy-, C₂-C₆-alkenyl, C₃-C₈-cycloalkyl, 4- to 7-membered heterocycloalkyl, —O—CH₂-4- to 7-membered heterocycloalkyl, C₁-C₆-alkylsulfonyl, wherein where R² stands for a halogen atom, the halogen atom is not Cl,
—C(O)—NR^{a}R^{b}, wherein R^{a} and R^{b} are both hydrogen atoms, —C(O)—O—R⁹, wherein R⁹ is a C₁-C₆-alkyl, or —CH₂—NR^{a}R^{b}, wherein R^{a} and R^{b} are both hydrogen atoms,
and w is 1 or 2,
and wherein
A2(R³)_{y} stands either for a hydrogen atom or
A2 has the same meanings as the substituent A1 and
R³ stands for
a hydrogen atom, a halogen atom, a hydroxy, cyano, nitro group, a substituent selected from a C₁-C₆-alkyl, C₂-C₆-alkenyl, C₂-C₆-alkynyl, C₃-C₈-cycloalkyl, C₄-C₈-cycloalkenyl, C₇-C₈-cycloalkynyl, 4- to 7-membered heterocycloalkyl, 5- to 10-membered heterocycloalkenyl, phenyl, heteroaryl or C₁-C₆-haloalkyl,
which substituent is optionally substituted, one, two or three times, identically or differently, with a substituent selected from:
a halogen atom, or a group selected from hydroxy, cyano, C₁-C₆-alkyl, C₁-C₆-alkoxy, C₁-C₆-haloalkyl, phenyl, —C(O)NRⁱR^{j}, wherein
Rⁱ and R^{j} are selected independently from a hydrogen atom or a C₁-C₆-alkyl, heteroaryl,
or with amino —NR^{k}R^{l}, wherein R^{k} and R^{l} are selected independently from
a hydrogen atom, a substituent selected from a C₁-C₆-alkyl, C₂-C₆-alkenyl, C₂-C₆-alkynyl, C₃-C₈-cycloalkyl, C₁-C₆-alkylsulfonyl, phenyl, heteroaryl, 4- to 7-membered heterocycloalkyl, which are optionally substituted
one, two or three times, identically or differently, with a substituent selected from C₁-C₆-haloalkyl, hydroxyl, phenyl, cyano, C₁-C₆-alkoxy, heteroaryl, wherein
the heteroaryl can optionally be substituted with a methyl group, or —CH₂—C(O)—R^{m}, wherein
R^{m} is a bicyclic heteroaryl, which can be partially hydrogenated, a C₁-C₆-alkoxy or a group —NRⁿR^{o}, in which
Rⁿ and R^{o} are selected independently from hydrogen, C₁-C₆-alkyl, phenyl, wherein the C₁-C₆-alkyl can optionally be substituted with a C₁-C₆-alkoxy or a phenyl, or
RⁿR^{o} stands for a 4- to 7-membered azacycloalkyl, bound via the nitrogen atom to the rest of the molecule and which optionally contains one more heteroatom selected from nitrogen and oxygen;
—C(═O)R^{p}, wherein R^{p} is selected from
the group of a C1-C6-alkoxy, a C1-C6-alkyl, which is optionally substituted, one, two or three times, identically or differently, with a substituent selected from hydroxyl or C₁-C₆-alkoxy, a mono- or bicyclic heteroaryl, a 4- to 7-membered heterocycloalkyl or R^{p} is a group —CH₂—NR^{q}R^{f}; wherein R^{q} and R^{f} are selected independently from hydrogen, phenyl or a C₁-C₆-alkyl, which may optionally be substituted up to threefold with fluorine,
or —NR^{s}R^{t}, wherein R^{s} and R^{t} combine with the nitrogen to form
a 4- to 7-membered azacycloalkyl, bound via the nitrogen atom to the rest of the molecule, or R^{s} and R^{t} combine with the nitrogen to form a 6- to 10-membered azaspirocycloalkyl, which both may contain up to 2 further heteroatoms selected from nitrogen and oxygen and which both are optionally substituted one, two or three times, identically or differently, with a substituent selected from: hydroxy, oxo (═O), C₁-C₆-alkyl, C₁-C₆-hydroxyalkyl, —C(═O)OR^{u}, wherein R^{u} is a C₁-C₆-alkyl, halogen, —N(C₁-C₆-alkyl)₂, —CH₂—N(C₁-C₆-alkyl)₂, —C(O)NR^{a}R^{b}, wherein R^{a} and R^{b} are selected independently from a hydrogen atom or a C₁-C₆-alkyl,
or a substituent selected from: —C(═O)R^{v}, —C(═O)NH₂, —C(═O)N(H)R^{v}, —C(═O)N(R^{v})R^{w} or —C(═O)OR^{v}, wherein
R^{v} and R^{w} represent, independently from each other, a group selected from hydrogen, C₁-C₄-alkyl, C₁-C₄-haloalkyl, phenyl, or a group —(CH₂)₂—NRₓR_{y}, wherein Rₓ and R_{y} independently from each other stand for hydrogen, a C₁-C₄-alkyl or a group —(CH₂)₂N(CH₃)₂;
or a substituent selected from: —NH₂, —NHR^{z}, —N(R^{z})R^{za}, —N(H)C(═O)R^{z}, —N(H)C(═O)OR^{z}, —N(H)S(═O)₂R^{z}, 4- to 7-membered heterocycloalkyl, heteroaryl, heterospirocycloalkyl, fused heterocycloalkyl or bridged heterocycloalkyl, wherein
R^{z} and R^{za} represent, independently from each other, a group selected from C₁-C₄-alkyl, C₁-C₄-haloalkyl and phenyl,
or a substituent selected from: —C₁-C₆-alkoxy-, C₁-C₆-haloalkoxy-, —O—(CH₂)ₛ—C₃-C₈-cycloalkyl, —O—(CH₂)ₛ-phenyl, —O—(CH₂)ₛ-heterocycloalkyl or —O—(CH₂)ₛ-heteroaryl, wherein s is 0, 1, 2 or 3,
or a substituent selected from: —S(═O)₂R^{z}, —S(═O)₂NH₂, —S(═O)₂NHR^{z} or —S(═O)₂N(R^{z})R^{za}, wherein R^{z} and R^{za} represent, independently from each other, a group selected from C₁-C₄-alkyl, C₁-C₄-haloalkyl or phenyl,
wherein y is 1, 2 or 3, and
L stands either for a bond or for —O—(CH₂)ₖ, wherein k is 0, 1, 2 or 3, or a group —CH═CH—(CH₂)ₙ, wherein n is 0, 1 or 2,
or a tautomer, an N-oxide, a hydrate, a solvate, or a salt thereof, or a mixture of same.

3. The compound according to claim 1, in which:
R¹ is selected from the list of the following substituents
H, *—OCH₃, —OC₂H₅, 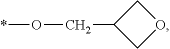 [SMILES: *OCC1COC1]
*—CH₂OH, *—C(O)OH, *—C(O)OCH₃, —Br, *—O—CH(CH₃)₂, *—O—(CH₂)₂CH(CH₃)₂, *—O—(CH₂)₃CH₃, *—O—(CH₂)₂O—CH₃, 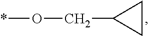 [SMILES: *OCC1CC1]
*—O—CH₂-Phenyl, *—N═S(O)(CH₃)₂, *—CH₃, 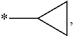 [SMILES: *C1CC1]
*—CH(CH₃), *—(CH₃)₂, *—CH₂, 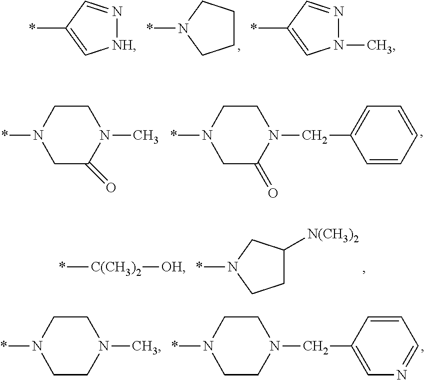
*—NH—(CH₂)₂—NH—C(O)—CH₃, *—NH—(CH₂)₂-morpholino, *—NH—C(O)—CH₃, *—NH—C(O)—NH—CH₃, *—NH—C(O)—N(CH₃)₂, —NO₂, *—NH—S(O)₂—CH₃, *—N═S(O)(CH₃)₂, —OH, *—O—(CH₂)₂—S(O)₂—CH₃, *-fluorine, 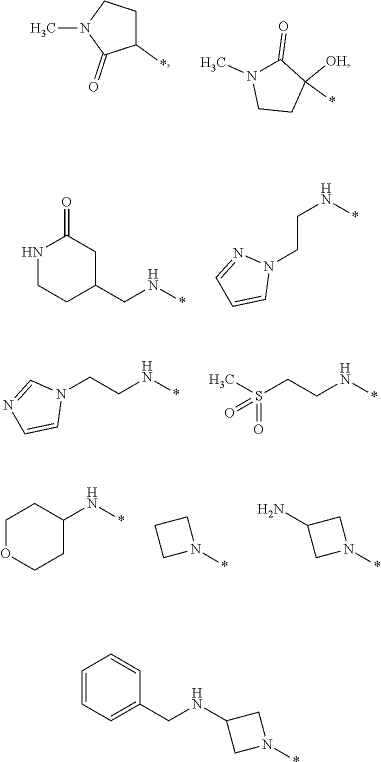 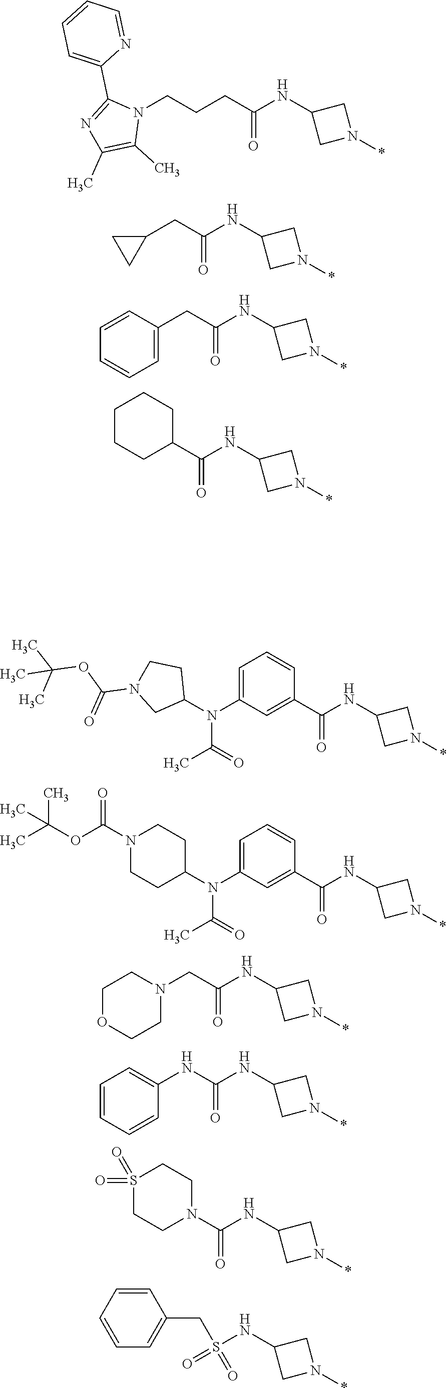 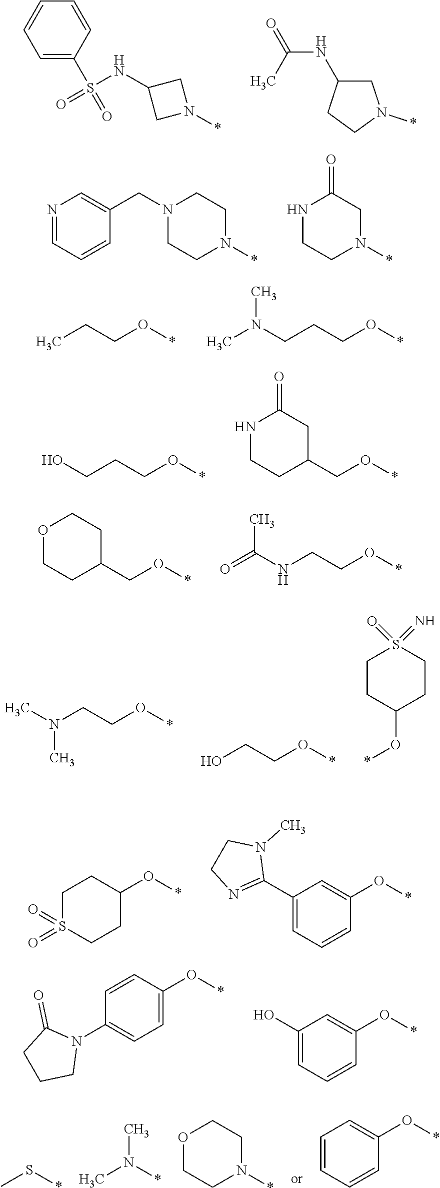
and
z is 1 or 2 and
x is 1 or 2 and wherein
A1 is 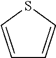
and
R² is selected from the group of ethyl, hydrogen, hydroxy, cyano, —C(═CH₂)CH₃, —C(CH₃)═CHCH₃, —CH═CH—(CH₂)₂CH₃, CH═CHCH₃, —C(O)NH₂, C(O)OCH₃, —S(O)₂CH₃, —OCH₃, —CH₂NH₂, a halogen atom, wherein where R² stands for a halogen atom, the halogen atom is not Cl, and
w is 1 or 2 and
A2 is selected from the group, hydrogen, and 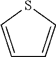
and
R³ is selected from the group of the following substituents
*—C(O)NH—(CH₂)₂CH₃
*—C(O)—N(CH₃)₂
*—C(O)—NH₂
*—C(O)—NH—(CH₂)₂N(CH₃)₂
*—CH₂—C(O)—NH₂
hydrogen
*—F, *—Cl, *—Br
*—C≡N; *—CF₃, *—CH₃, *—C₂H₅, *—CH═CH₂;
*—CH₂—CN; *—CH(CH₃)—NH₂; *—CH═CH—CN;
*—C(O)—OH; *—C(O)—OCH₃; *—C(O)—CH₃; *—C(CH₃)₂—C(O)—OCH₃; *—C(CH₃)₂—CN; hydroxy; 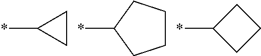
*—NH₂
*—NH—C(O)CH₃
*—NH—SO₂—CH₃
*—NH—C(O)—O—C(CH₃)₃ 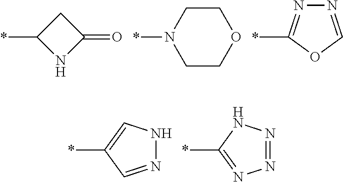
*—SO₂—CH₃
*—SO₂—N(CH₃)₂
*—SO₂—NH₂
*—O—CH₂—CH₃; *—O—(CH₂)₂—CH₃; *—O—CF₃; 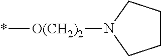
—OCH₂—Cyclopropyl; *—OCH₃;
*—O(CH₂)₃—CH₃; *—OCH₂-Phenyl; *—O-Phenyl;
*—(CH₂)—OH
*—(CH₂)₂—OH
*—(CH₂)—O—CH₃
*—(CH₂)—O—CH₂—CH₃
*—CH(OH)—CH₂-Phenyl
*—CH(OH)—CH₂—CH₃
*—CH(OH)—(CH₂)₂—CH₃; *—CH(OH)—(CH₂)₃—CH₃
*—CH(OH)—CH—(CH₃)₂
*—CH(OH)-Phenyl
*—CH(OH)—CN
*—CH(OH)—CH₂OH
*—CH(OH)—CF₃
*—CH(OH)—(CH₂)₂-Phenyl
*—CH(OH)—C≡CH
*—CH(NH₂)—CH₂—COOH
*—CH₂—NH—SO₂—CH₃
*—CH₂—NH—(CH₂)₃—CH₃
*—CH₂—NH—CH₃
*—CH₂—N(CH₃)₂
*—CH₂—NH—C₂H₅; *—CH(CH₃)—NH₂
*—CH₂—NH₂
*—(CH₂)₂—NH₂
*—CH₂—NH—CH₂-Phenyl
*—CH₂—N(C₂H₅)₂
*—CH₂—NH-Cyclopropyl
*—CH₂—NH-Cyclobutyl
*—CH₂—NH-Cyclopentyl
*—CH₂—NH-Pyridyl
*—CH₂—NH-Phenyl
*—CH₂—NH—(CH₂)₂—OH
*—CH₂—N(CH₃)(CH₂)₂OH; *—CH₂—NH—CH₂—CN
*—CH₂—N(CH₃)—CH₂—CN
*—CH₂— N(CH₂)—CH₂—CF₃
*—CH₂—N(CH₃)—CH₂—CF₂H
*—CH₂—NH—CH₂—CF₂H
*—CH₂—NH—CH₂—CF₃
*—CH₂—NH—(CH₂)₂—OCH₃ 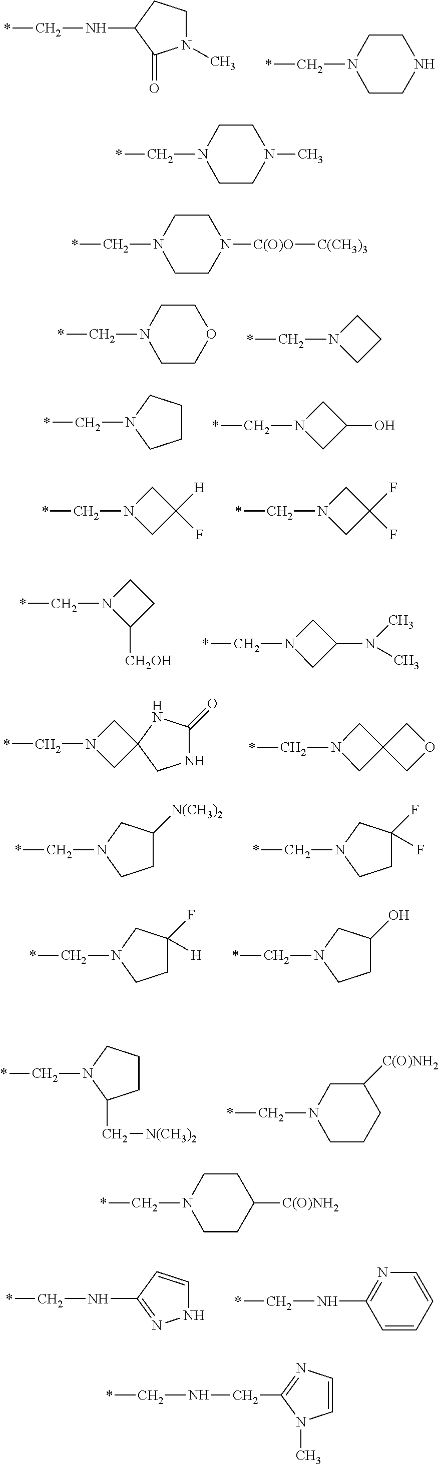
*—CH₂—NH—C(O)—O—C(CH₃)₃
*—(CH₂)₂—NH—C(O)—O—C(CH₃)₃
*—CH₂—NH—C(O)—CH₂—OH
*—CH₂—NH—C(O)—CH₂—OCH₃
*—CH—(CH₃)—NH—C(O)—O—C(CH₃)₃
*—CH₂—NH—C(O)—CH₃ 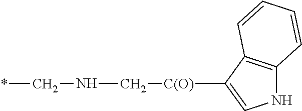
CH₂—NH—CH₂—C(O)—NH₂
—CH₂—NH—CH₂—C(O)—N(CH₃)₂
—CH₂—NH—CH₂—C(O)—OCH₃
—CH₂—NH—CH₂—C(O)—NHCH₃
CH₂—NH—CH₂—C(O)—NH—(CH₂)₂—O—CH₃
CH₂—NH—CH₂—C(O)—NH—CH₂-Phenyl 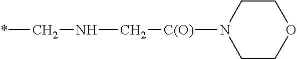
*—CH₂—NH—CH₂—C(O)—NH-Phenyl 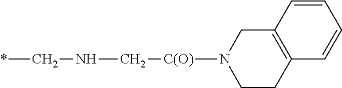
*—CH₂—NH—C(O)—CH₂—NH-Phenyl 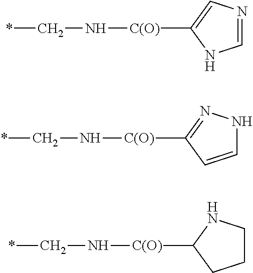
*—CH₂—NH—C(O)—CH₂—NH—CH₂—CF₃; and 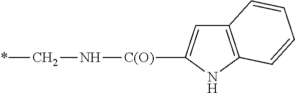
and
y is 1 or 2 and
k is 1 or 2 and
n is 0 or 1
and stereoisomers, tautomers, N-oxides, hydrates, solvates, and salts thereof, and mixtures of same.

4. The compound according to claim 3, wherein R¹ is selected from the list of the following substituents H, *—OCH₃, *—OC₂H₅, 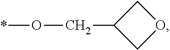 [SMILES: *OCC1COC1]
*—CH₂OH, *—C(O)OH, *—C(O)OCH₃, —Br, —O—CH(CH₃)₂, *—O—(CH₂)₂CH(CH₃)₂, *—O—(CH₂)₃CH₃, *—O—(CH₂)₂O—CH₃, 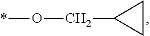 [SMILES: *OCC1CC1]
*—O—CH₂-Phenyl, *—N═S(O)(CH₃)₂, *—CH₃, 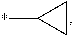 [SMILES: *C1CC1]
*—NH(CH₃), *—N(CH₃)₂, *—NH₂, 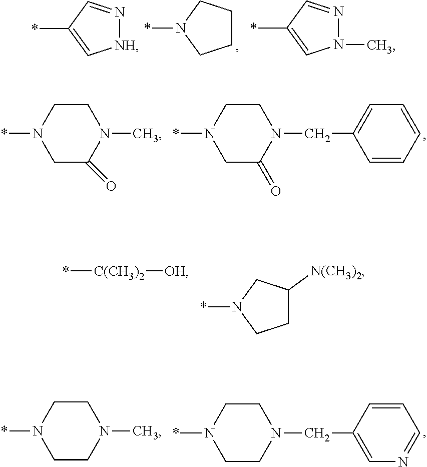
*—NH—(CH₂)₂—NH—C(O)—CH₃, *—NH—(CH₂)₂-morpholino, *—NH—C(O)—CH₃, *—NH—C(O)—NH—CH₃, *—NH—C(O)—N(CH₃)₂, *—NO₂, *—NH—S(O)₂—CH₃, *—N═S(O)(CH₃)₂, or *—OH, *—O—(CH₂)₂—S(O)₂—CH₃.

5. The compound according to claim 1 wherein the carbon atom between the nitrogen atom and the substituent A1 is in R-configuration.

6. The compound according to claim 1, wherein R¹ is selected from the list of the following substituents H, *—OCH₃, *—OC₂H₅, 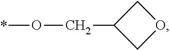 [SMILES: *OCC1COC1]
*—CH₂OH, *—C(O)OH, *—C(O)OCH₃, —Br, —O—CH(CH₃)₂, —O—(CH₂)₂CH(CH₃)₂, —O—(CH₂)₃CH₃, —O—(CH₂)₂O—CH₃, 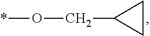 [SMILES: *OCC1CC1]
—O—CH₂-Phenyl, —N═S(O)(CH₃)₂, —CH₃, 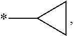 [SMILES: *C1CC1]
—NH(CH₃), —N(CH₃)₂, —NH₂, 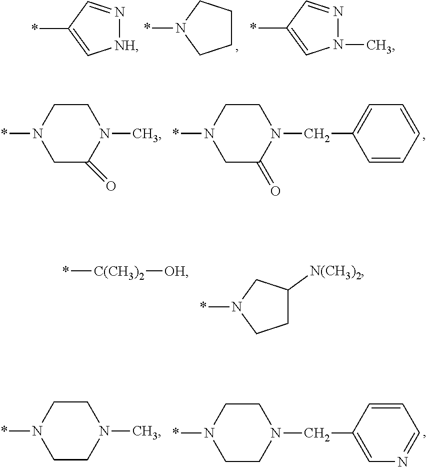
—NH—(CH₂)₂—NH—C(O)—CH₃, —NH—(CH₂)₂-morpholino, —NH—C(O)—CH₃, —NH—C(O)—NH—CH₃, —NH—C(O)—N(CH₃)₂, —NO₂, —NH—S(O)₂—CH₃, —N═S(O)(CH₃)₂, —OH, —O—(CH₂)₂—S(O)₂—CH₃, *-fluorine,

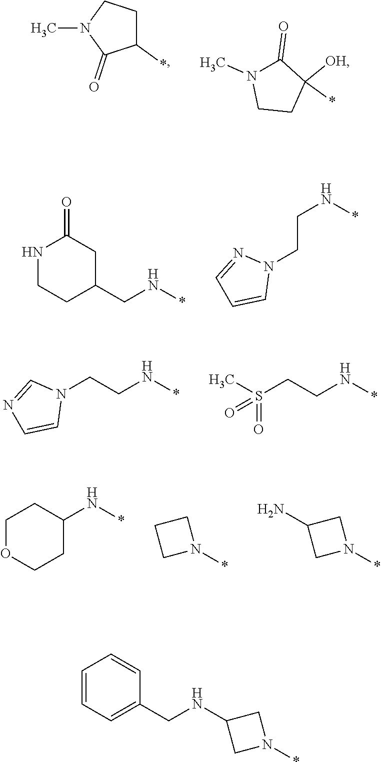

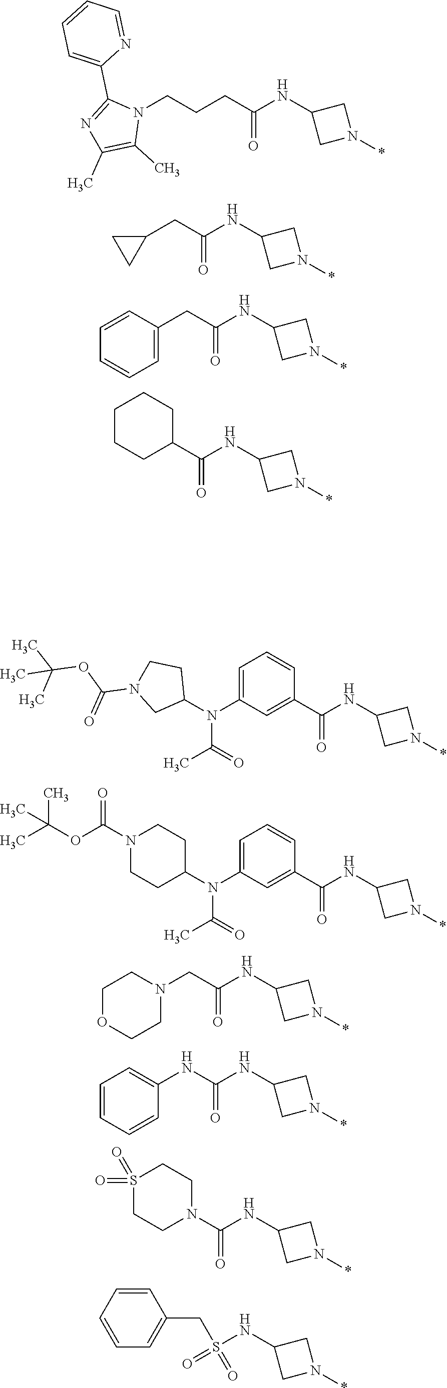

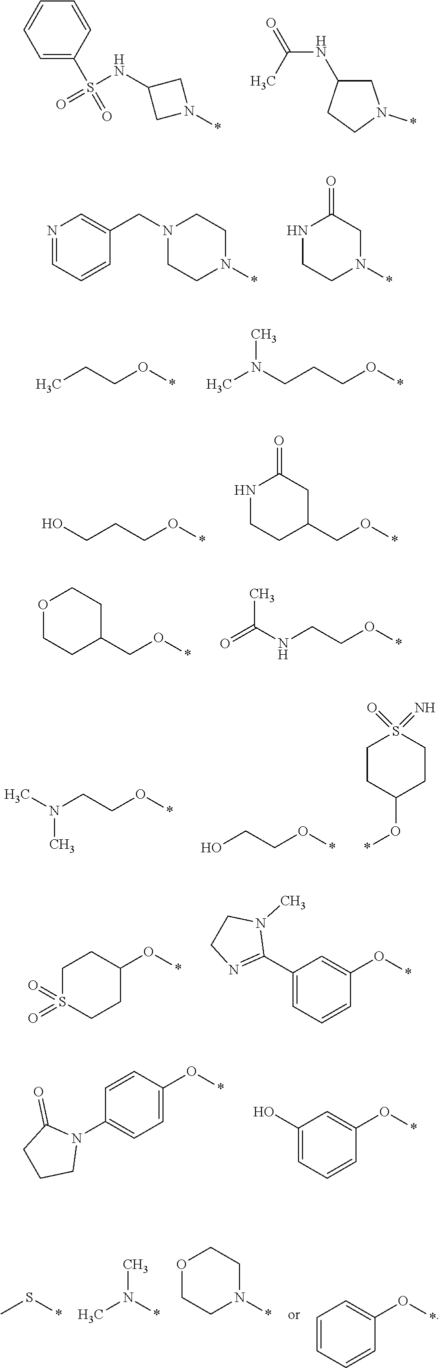

7. The compound according to claim 6, wherein R¹ is selected from the list of the following substituents H, —OCH₃, —OC₂H₅, 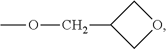
—CH₂OH, —C(O)OH, —C(O)OCH₃, —Br, —O—CH(CH₃)₂, —O—(CH₂)₂CH(CH₃)₂, —O—(CH₂)₃CH₃, —O—(CH₂)₂O—CH₃, 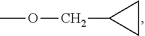
—O—CH₂-Phenyl, —N═S(O)(CH₃)₂, —CH₃, 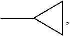
—NH(CH₃), —N(CH₃)₂, —NH₂, 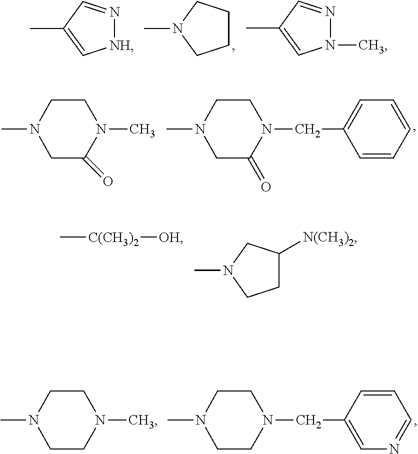
—NH—(CH₂)₂—NH—C(O)—CH₃, —NH—(CH₂)₂-morpholino, —NH—C(O)—CH₃, —NH—C(O)—NH—CH₃, —NH—C(O)—N(CH₃)₂, —NO₂, —NH—S(O)₂—CH₃, —N═S(O)(CH₃)₂, —OH, or —O—(CH₂)₂—S(O)₂—CH₃.

8. The compound according to claim 1, wherein R² is selected from the group of hydrogen, hydroxyl, —C(═CH₂)CH₃, —C(CH₃)═CHCH₃, —CH═CH—(CH₂)₂CH₃, CH═CHCH₃, —C(O)NH₂, C(O)OCH₃, —S(O)₂CH₃, —OCH₃, —CH₂NH₂ and a halogen atom, wherein when R² stands for a halogen atom it is not Cl.

9. The compound according to claim 1, wherein A2 is

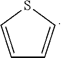

10. The compound according to claim 1, wherein A2 is hydrogen.

11. The compound according to claim 1, wherein R³ is selected from the group of the following substituents
*—O(O)NH—(CH₂)₂CH₃
*—C(O)—N(CH₃)₂
*—C(O)—NH₂
*—C(O)—NH—(CH₂)₂N(CH₃)₂
*—CH₂—C(O)—NH₂
hydrogen
*—F, *—Cl, *—Br
*—C≡N; *—CF₃, *—CH₃, *—C₂H₅, *—CH═CH₂;
*—CH₂—CN; *—CH(CH₃)—NH₂; *—CH═CH—CN;
—C(O)—OH; *—C(O)—OCH₃; *—C(O)—CH₃; *—C(CH₃)₂—C(O)—OCH₃; *—C(CH₃)₂—CN; hydroxy; 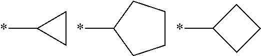
*—NH₂
*—NH—C(O)CH₃
*—NH—SO₂—CH₃
*—NH—C(O)—O—C(CH₃)₃ 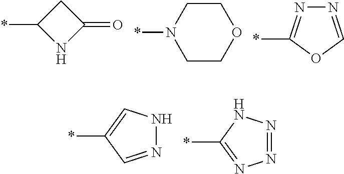
*—SO₂—CH₃
*—SO₂—N(CH₃)₂
*—SO₂—NH₂
*—O—CH₂—CH₃; *—O—(CH₂)₂—CH₃; *—O—CF₃; 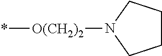
*—OCH₂-Cyclopropyl; *—OCH₃;
*—O(CH₂)₃—CH₃; *—OCH₂-Phenyl; *—O-Phenyl;
*—(CH₂)—OH
*—(CH₂)₂—OH
*—(CH₂)—O—CH₃
*—(CH₂)—O—CH₂—CH₃
*—CH(OH)—CH₂-Phenyl
*—CH(OH)—CH₂—CH₃
*—CH(OH)—(CH₂)₂—CH₃; *—CH(OH)—(CH₂)₃—CH₃
*—CH(OH)—CH—(CH₃)₂
*—CH(OH)-Phenyl
*—CH(OH)—CN
*—CH(OH)—CH₂OH
*—CH(OH)—CF₃
*—CH(OH)—(CH₂)₂-Phenyl
*—CH(OH)—C≡CH
*—CH(NH₂)—CH₂—COOH
*—CH₂—NH—SO₂—CH₃
*—CH₂—NH—(CH₂)₃—CH₃
*—CH₂—NH—CH₃
*—CH₂—N(CH₃)₂
*—CH₂—NH—C₂H₅; *—CH(CH₃)—NH₂
*—CH₂—NH₂
*—(CH₂)₂—NH₂
*—CH₂—NH—CH₂-Phenyl
*—CH₂—N(C₂H₅)₂
*—CH₂—NH-Cyclopropyl
*—CH₂—NH-Cyclobutyl
*—CH₂—NH-Cyclopentyl
*—CH₂—NH-Pyridyl
*—CH₂—NH-Phenyl
*—CH₂—NH—(CH₂)₂—OH
*—CH₂—N(CH₃)(CH₂)₂OH; *—CH₂—NH—CH₂—CN
*—CH₂—N(CH₃)—CH₂—CN
*—CH₂—N(CH₃)—CH₂—CF₃
*—CH₂—N(CH₃)—CH₂—CF₂H
*—CH₂—NH—CH₂—CF₂H
*—CH₂—NH—C(O)—O—C(CH₃)₃
*—(CH₂)₂—NH—C(O)—O—C(CH₃)₃ 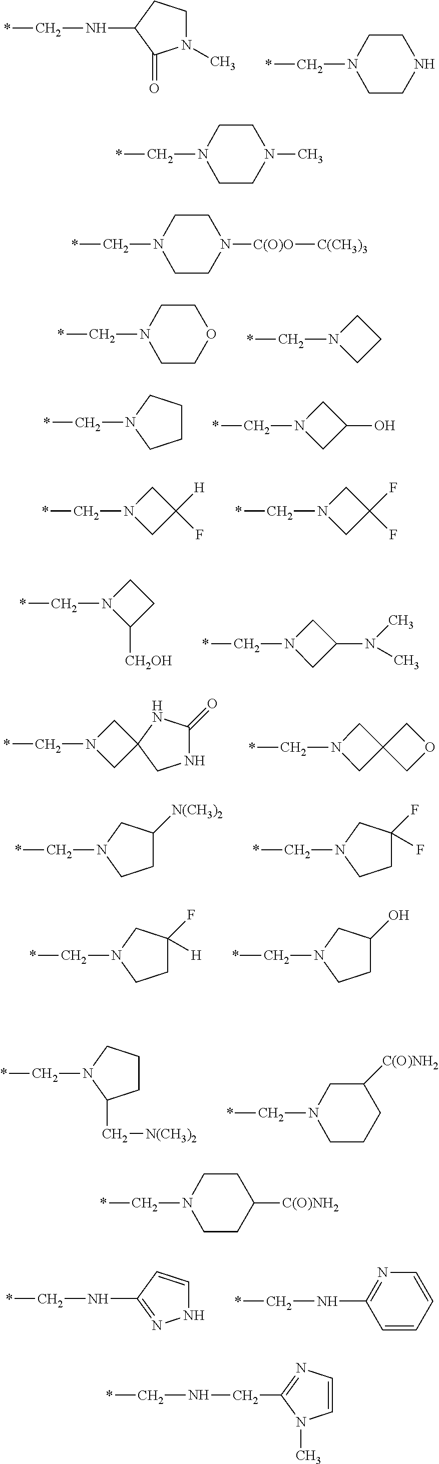
*—CH₂—NH—C(O)—O—C(CH₃)₃
*—(CH₂)₂—NH—C(O)—O—C(CH₃)₃
*—CH₂—NH—C(O)—CH₂—OH
*—CH₂—NH—C(O)—CH₂—OCH₃
*—CH—(CH₃)—NH—C(O)—O—C(CH₃)₃
*—CH₂—NH—C(O)—CH₃ 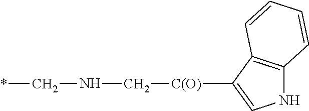
*—CH₂—NH—CH₂—C(O)—NH₂
*—CH₂—NH—CH₂—C(O)—N(CH₃)₂
*—CH₂—NH—CH₂—C(O)—OCH₃
*—CH₂—NH—CH₂—C(O)—NHCH₃
*—CH₂—NH—CH₂—C(O)—NH—(CH₂)₂—O—CH₃
*—CH₂—NH—CH₂—C(O)—NH—CH₂-Phenyl 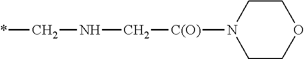
*—CH₂—NH—CH₂—C(O)—NH-Phenyl 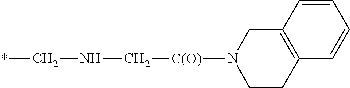
*—CH₂—NH—C(O)—CH₂—NH-Phenyl 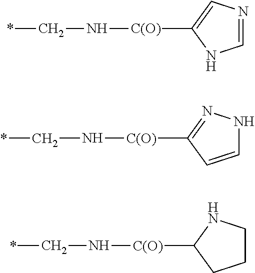
*—CH₂—NH—C(O)—CH₂—NH—CH₂—CF₃;

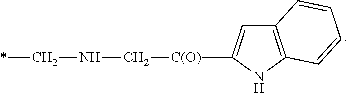

12. The compound according to claim 1, wherein R³ is a C₁- or C₂-alkyl substituted with an amino group —NR^{k}R^{l}, wherein Rₖ and R^{l} have the meanings as defined in claim 1.

13. The compound according to claim 1, wherein R³ is a C₁- or C₂-alkyl substituted with a hydroxyl or a C₁-C₆-alkoxy.

14. The compound according to claim 1, wherein x is 1 or 2.

15. The compound according to claim 1, wherein y is 1 or 2.

16. The compound according to claim 1, wherein z is 1 or 2.

17. A pharmaceutical composition comprising a compound of general formula (I) according to claim 1.

18. A pharmaceutical composition comprising a compound of general formula (I) according to claim 1 and one or more pharmaceutically acceptable excipients.

19. A method for preparing a medicament comprising a compound of general formula (I) according to claim 1, the method comprising mixing the compound of general formula (I) with one or more pharmaceutically acceptable excipients.
